SEQUENCE LISTING (112 sequences)

SEQ ID NO: 1 - protein, 106 aa, Artificial Sequence [sequence omitted]

SEQ ID NO: 2 - protein, 122 aa, Artificial Sequence [sequence omitted]

SEQ ID NO: 3 - protein, 107 aa, Artificial Sequence [sequence omitted]

SEQ ID NO: 4 - protein, 120 aa, Artificial Sequence [sequence omitted]

SEQ ID NO: 5 - protein, 108 aa, Artificial Sequence
  Anti-CTLA-4 variable light chain (VL)
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIK

SEQ ID NO: 6 - protein, 118 aa, Artificial Sequence
  Anti-CTLA-4 variable heavy chain (VH)
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSS

SEQ ID NO: 7 - protein, 107 aa, Artificial Sequence [sequence omitted]

SEQ ID NO: 8 - protein, 330 aa, Artificial Sequence [sequence omitted]

SEQ ID NO: 9 - protein, 326 aa, Artificial Sequence
  IgG2 constant heavy chain (CH1-hinge-CH2-CH3)
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 10 - protein, 377 aa, Artificial Sequence
  IgG3 constant heavy chain (CH1-hinge-CH2-CH3)
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 11 - protein, 327 aa, Artificial Sequence
  IgG4 constant heavy chain (CH1-hinge-CH2-CH3)
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 106 aa, Artificial Sequence [sequence omitted]

SEQ ID NO: 13 - protein, 121 aa, Artificial Sequence [sequence omitted]

SEQ ID NO: 14 - protein, 106 aa, Artificial Sequence [sequence omitted]

SEQ ID NO: 15 - protein, 122 aa, Artificial Sequence [sequence omitted]

SEQ ID NO: 16 - protein, 107 aa, Artificial Sequence [sequence omitted]

SEQ ID NO: 17 - protein, 120 aa, Artificial Sequence [sequence omitted]

SEQ ID NO: 18 - protein, 111 aa, Artificial Sequence
  Anti-CD30 L3.71 AC10 variable light chain (VL)
EIVLTQSPDSLAVSLGERATINCKASQSVDFDGDSYLNWYQQKPGQPPKVLIYAASTLQSGVPSRFSGSGSGTDFTLTINSLEAEDAATYYCQQSNEDPWTFGGGTKVEIK

SEQ ID NO: 19 - protein, 117 aa, Artificial Sequence
  Anti-CD30 H3.69_V2 AC10 variable heavy chain (VH)
QVQLVQSGAEVKKPGASVKVSCKVSGYTFTDYYITWVRQAPGQALEWMGWIYPGSGNTKYSQKFQGRFVFSVDTSASTAYLQISSLKAEDTAVYYCANYGNYWFAYWGQGTLVTVSS

SEQ ID NO: 20 - protein, 329 aa, Artificial Sequence
  IgG(1/2) constant heavy chain (CH1-hinge-CH2-CH3)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 21 - protein, 330 aa, Artificial Sequence
  IgG(1/2) ELLGG constant heavy chain (CH1-hinge-CH2-CH3)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 22 - protein, 213 aa, Artificial Sequence
  Anti-CD20 light chain (VL-CL)
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 451 aa, Artificial Sequence
  Anti-CD20 heavy chain (VH-CH1-hinge-CH2-CH3)
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVQFNWYVDGVEVHNAKTKPREEQFNSTFRVVSVLTVVHQDWLNGKEYKCKVSNKALPAPEEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 24 - protein, 218 aa, Artificial Sequence
  Anti-CD30 light chain (VL-CL)
EIVLTQSPDSLAVSLGERATINCKASQSVDFDGDSYLNWYQQKPGQPPKVLIYAASTLQSGVPSRFSGSGSGTDFTLTINSLEAEDAATYYCQQSNEDPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 447 aa, Artificial Sequence
  Anti-CD30 heavy chain (VH-CH1-hinge-CH2-CH3)
QVQLVQSGAEVKKPGASVKVSCKVSGYTFTDYYITWVRQAPGQALEWMGWIYPGSGNTKYSQKFQGRFVFSVDTSASTAYLQISSLKAEDTAVYYCANYGNYWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVQFNWYVDGVEVHNAKTKPREEQFNSTFRVVSVLTVVHQDWLNGKEYKCKVSNKALPAPEEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 26 - protein, 106 aa, Artificial Sequence [sequence omitted]

SEQ ID NO: 27 - protein, 122 aa, Artificial Sequence [sequence omitted]

SEQ ID NO: 28 - protein, 107 aa, Artificial Sequence
  Anti-EGFR L3.32 C225 variable light chain (VL)
DIQLTQSPSSLSASVGDRVTITCRASQSISSNLHWYQQKPDQSPKLLIKYASESISGVPSRFSGSGSGTDFTLTISSLQAEDVAVYYCQQNNNWPTTFGQGTKLEIK

SEQ ID NO: 29 - protein, 119 aa, Artificial Sequence
  Anti-EGFR H4.40 C225 variable heavy chain (VH)
QVQLQQSGPGLVKPSQTLSLTCTVSGFSLSNYGVHWVRQAPGKGLEWMGIIWSGGSTDYNTSLKSRLTISKDTSKSQVVLTMTNMDPVDTATYYCARALTYYDYEFAYWGQGTLVTVSS

SEQ ID NO: 30 - protein, 107 aa, Artificial Sequence
  Anti-EpCAM L3 17-1A variable light chain (VL)
NIVMTQSPDSLAVSLGERATLSCRASENVVTYVSWYQQKPGQSPQLLIYGASNRYTGVPDRFTGSGSATDFTLTINSLEAEDAATYYCGQGYSYPYTFGGGTKLEIK

SEQ ID NO: 31 - protein, 116 aa, Artificial Sequence
  Anti-EpCAM H3.77 17-1A heavy chain (VH)
EVQLVESGGGLVQPGGSLRLSCAASGYSFTNYLIEWVRQAPGQGLEWMGVINPGSGGTNYNPSLKSRVTISADKSISTAYMELSSLRSEDTAVYFCARDGPWFAYWGQGTLVTVSS

SEQ ID NO: 32 - protein, 111 aa, Artificial Sequence; identical to SEQ ID NO: 18
  Anti-CD30 L3.71 AC10 variable light chain (VL)

SEQ ID NO: 33 - protein, 117 aa, Artificial Sequence; identical to SEQ ID NO: 19
  Anti-CD30 H3.69_V2 AC10 variable heavy chain (VH)

SEQ ID NO: 34 - protein, 107 aa, Artificial Sequence
  Murine kappa constant light chain
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRGEC

SEQ ID NO: 35 - protein, 324 aa, Artificial Sequence
  Murine IgG1 constant heavy chain (CH1-hinge-CH2-CH3)
AKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDPEVQFSWFVDDVEVHTAQTKPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 36 - protein, 330 aa, Artificial Sequence
  Murine IgG2a allele a constant heavy chain (CH1-hinge-CH2-CH3)
AKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPMVTCVVVDVSEDDPDVQISWFVNNVEVLTAQTQTHREDYNSTLRVVSALPIQHQDWMSGKEFKCKVNNKALPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 37 - protein, 335 aa, Artificial Sequence
  Murine IgG2a allele b constant heavy chain (CH1-hinge-CH2-CH3)
AKTTAPSVYPLAPVCGGTTGSSVTLGCLVKGYFPEPVTLTWNSGSLSSGVHTFPALLQSGLYTLSSSVTVTSNTWPSQTITCNVAHPASSTKVDKKIEPRVPITQNPCPPLKECPPCAAPDLLGGPSVFIFPPKIKDVLMISLSPMVTCVVVDVSEDDPDVQISWFVNNVEVHTAQTQTHREDYNSTLRVVSALPIQHQDWMSGKEFKCKVNNRALPSPIEKTISKPRGPVRAPQVYVLPPPAEEMTKKEFSLTCMITGFLPAEIAVDWTSNGRTEQNYKNTATVLDSDGSYFMYSKLRVQKSTWERGSLFACSVVHEGLHNHLTTKTISRSLGK

SEQ ID NO: 38 - protein, 336 aa, Artificial Sequence
  Murine IgG2b constant heavy chain (CH1-hinge-CH2-CH3)
AKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVQISWFVNNVEVHTAQTQTHREDYNSTIRVVSALPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVRHEGLKNYYLKKTISRSPGK

SEQ ID NO: 39 - protein, 330 aa, Artificial Sequence
  Murine IgG3 constant heavy chain (CH1-hinge-CH2-CH3)
ATTTAPSVYPLVPGCGDTSGSSVTLGCLVKGYFPEPVTVKWNYGALSSGVRTVSSVLQSGFYSLSSLVTVPSSTWPSQTVICNVAHPASKTELIKRIEPRIPKPSTPPGSSCPPGNILGGPSVFIFPPKPKDALMISLTPKVTCVVVDVSEDDPDVHVSWFVDNKEVHTAWTQPREAQYNSTFRVVSALPIQHQDWMRGKEFKCKVNNKALPAPIERTISKPKGRAQTPQVYTIPPPREQMSKKKVSLTCLVTNFFSEAISVEWERNGELEQDYKNTPPILDSDGTYFLYSKLTVDTDSWLQGEIFTCSVVHEALHNHHTQKNLSRSPGK

SEQ ID NO: 40 - protein, 330 aa, Artificial Sequence
  IgG1 constant heavy chain (CH1-hinge-CH2-CH3) comprising possible Fc variants
  MISC_FEATURE (117)..(117): Xaa can be Gly or His
  MISC_FEATURE (118)..(118): Xaa can be Asp, Gly or His
  MISC_FEATURE (119)..(119): Xaa can be Ile, Asn, Pro or Arg
  MISC_FEATURE (120)..(120): Xaa can be Lys or Leu
  MISC_FEATURE (150)..(150): Xaa can be Arg
  MISC_FEATURE (152)..(152): Xaa can be Arg
  MISC_FEATURE (208)..(208): Xaa can be Ala or Leu
  MISC_FEATURE (211)..(211): Xaa can be Arg
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEXXXXPSVFLFPPKPKDTLMISRTPEVTCVVVDVXHXDPEVKFNWYVDGVEVHNAKTKPREEQYNSTYRVVSVLTVLHQDWLNGKEYKCKVSXKAXPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - protein, 215 aa, Artificial Sequence
  Anti-CTLA-4 light chain (VL-CL)
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 42 - protein, 448 aa, Artificial Sequence
  Anti-CTLA-4 L235G/G236R IgG1 heavy chain (VH-CH1-hinge-CH2-CH3)
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELGRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - protein, 444 aa, Artificial Sequence
  Anti-CTLA-4 IgG2 heavy chain (VH-CH1-hinge-CH2-CH3)
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 44 - protein, 451 aa, Artificial Sequence
  Anti-CD20 heavy chain comprising possible Fc variants
  MISC_FEATURE (243)..(243): Xaa can be Asp, Glu, Asn, Gln or Thr
  MISC_FEATURE (244)..(244): Xaa can be Ile or Met
  MISC_FEATURE (268)..(268): Xaa can be Ile, Thr or Tyr
  MISC_FEATURE (272)..(272): Xaa can be Asp or Glu
  MISC_FEATURE (276)..(276): Xaa can be Tyr
  MISC_FEATURE (278)..(278): Xaa can be Glu
  MISC_FEATURE (301)..(301): Xaa can be Asp
  MISC_FEATURE (302)..(302): Glx can be Ala or Asp
  MISC_FEATURE (330)..(330): Glx can be Glu or Thr
  MISC_FEATURE (334)..(334): Xaa can be Tyr, Leu or Ile
  MISC_FEATURE (336)..(336): Xaa can be Asp, Glu, Asn or Gln
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPXXFLFPPKPKDTLMISRTPEVTCVVXDVSXEDPXVXFNWYVDGVEVHNAKTKPREEQYXZTYRVVSVLTVLHQDWLNGKEYKCKVSNZALPXPXEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - protein, 213 aa, Artificial Sequence; identical to SEQ ID NO: 22
  Anti-CD20 light chain

SEQ ID NO: 46 - protein, 451 aa, Artificial Sequence
  Anti-CD20 heavy chain
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 117 aa, Artificial Sequence
  Fc variant
  MISC_FEATURE (1)..(1): Xaa can be Asp, Lys or Tyr
  MISC_FEATURE (2)..(2): Xaa can be Lys, Glu or Tyr
  MISC_FEATURE (3)..(3): Xaa can be Thr, Glu or Lys
  MISC_FEATURE (4)..(4): Xaa can be His, Glu or Tyr
  MISC_FEATURE (5)..(5): Xaa can be Thr, Glu, Lys or Trp
  MISC_FEATURE (7)..(8): Xaa can be Pro, Glu, Gly, Lys or Tyr
  MISC_FEATURE (10)..(10): Xaa can be Pro, Ala, Glu, Gly or Tyr
  MISC_FEATURE (11)..(11): Xaa can be Ala, Glu, Gly, Lys, Pro or Tyr
  MISC_FEATURE (12)..(12): Xaa can be Pro, Glu, Gly, Lys or Tyr
  MISC_FEATURE (13)..(13): Xaa can be Pro, Glu, Gly, Lys or Tyr
  MISC_FEATURE (14)..(16): Xaa can be any amino acid except Cys
  MISC_FEATURE (17)..(19): Xaa can be any amino acid except Ala or Cys
  MISC_FEATURE (20)..(20): Xaa can be Val, Ala, Ile, Met or Thr
  MISC_FEATURE (21)..(21): Xaa can be Phe, Asp, Glu, Leu, Arg, Ser, Trp or Tyr
  MISC_FEATURE (23)..(23): Xaa can be Phe, Glu, His, Leu, Gln, Arg, Trp or Tyr
  MISC_FEATURE (24)..(24): Xaa can be Pro or His
  MISC_FEATURE (25)..(25): Xaa can be Pro or Ala
  MISC_FEATURE (26)..(26): Xaa can be Lys, Asp, Glu, His or Tyr
  MISC_FEATURE (27)..(27): Xaa can be Pro, Gly or Val
  MISC_FEATURE (29)..(29): Xaa can be Asp, His, Gln or Tyr
  MISC_FEATURE (35)..(35): Xaa can be Arg, Glu or Tyr
  MISC_FEATURE (38)..(38): Xaa can be Glu, His, Ser or Tyr
  MISC_FEATURE (40)..(40): Xaa can be Thr, Asp, Glu, His or Tyr
  MISC_FEATURE (42)..(42): Xaa can be Val, Ala, Glu, Phe, Ile or Thr
  MISC_FEATURE (43)..(43): Xaa can be Val, Ala, Ile, Met or Thr
  MISC_FEATURE (44)..(44): Xaa can be any amino acid except Cys
  MISC_FEATURE (45)..(45): Xaa can be any amino acid except Cys, Ala, or Glu
  MISC_FEATURE (46)..(46): Xaa can be any amino acid except Cys, Ala, or Glu
  MISC_FEATURE (47)..(47): Xaa can be any amino acid except Cys, Ala, or Gly
  MISC_FEATURE (48)..(48): Xaa can be any amino acid except Cys, Ala, or Ser
  MISC_FEATURE (49)..(49): Xaa can be any amino acid except Cys, Ala, Asp or Gln
  MISC_FEATURE (50)..(50): Xaa can be any amino acid except Cys, Ala, Glut, Asn, Lys or Val
  MISC_FEATURE (51)..(51): Xaa can be any amino acid except Cys or Pro
  MISC_FEATURE (52)..(52): Xaa can be any amino acid except Cys, Ala, Arg or Gln
  MISC_FEATURE (53)..(53): Xaa can be Val or Ile
  MISC_FEATURE (54)..(54): Xaa can be any amino acid except Ala, Cys, Gln, Ile or Ser
  MISC_FEATURE (55)..(55): Xaa can be Phe, Leu or Trp
  MISC_FEATURE (56)..(56): Xaa can be any amino acid except Ala, Cys, Gln or Lys
  MISC_FEATURE (58)..(58): Xaa can be any amino acid except Ala, Cys or Phe
  MISC_FEATURE (60)..(60): Xaa can be Asp, Gly, Lys, Leu, Pro or Trp
  MISC_FEATURE (61)..(61): Xaa can be Asp, Gly, Glu, Lys, Asn, Pro, Gln or Tyr
  MISC_FEATURE (62)..(62): Xaa can be Val, Glu, Gly, Lys, Pro or Tyr
  MISC_FEATURE (63)..(63): Xaa can be Glu, Gly, His, Lys, Pro, Arg, Tyr or Leu
  MISC_FEATURE (64)..(64): Xaa can be Val, Asp, Glu, Leu, Asn, Gln, Thr or Tyr
  MISC_FEATURE (65)..(65): Xaa can be His, Asp, Glu, Lys, Gln, Trp or Tyr
  MISC_FEATURE (66)..(66): Xaa can be Asn, Glu, Gly, Pro or Tyr
  MISC_FEATURE (68)..(68): Xaa can be Lys, Asp, Glu or Tyr
  MISC_FEATURE (70)..(70): Xaa can be Lys, Asp, His, Leu, Asn or Trp
  MISC_FEATURE (71)..(71): Xaa can be Lys, Asp, His, Leu, Asn or Trp
  MISC_FEATURE (72)..(72): Xaa can be Arg, Asp, Glu, Thr or Tyr
  MISC_FEATURE (73)..(73): Xaa can be any amino acid except Ala, Cys, Asp, Gln or Lys
  MISC_FEATURE (74)..(74): Xaa can be any amino acid except Ala, Cys, Arg, Asp or Gln
  MISC_FEATURE (75)..(75): Xaa can be any amino acid except Ala, Cys, Leu or Lys
  MISC_FEATURE (76)..(76): Xaa can be any amino acid except Cys, Phe, Pro or Trp
  MISC_FEATURE (77)..(77): Xaa can be any amino acid except Ala or Cys
  MISC_FEATURE (78)..(78): Xaa can be any amino acid except Ala, Cys, Gly, Leu, Pro or Val
  MISC_FEATURE (79)..(79): Xaa can be any amino acid except Cys
  MISC_FEATURE (80)..(80): Xaa can be any amino acid except Cys, Ile, Leu or Phe
  MISC_FEATURE (81)..(81): Xaa can be Arg, Asp, Glu, His or Tyr
  MISC_FEATURE (82)..(82): Xaa can be Val or Ile
  MISC_FEATURE (83)..(83): Xaa can be Val, Asp, Glu or Tyr
  MISC_FEATURE (84)..(84): Xaa can be Ser, Asp, His, Leu, Asn or Thr
  MISC_FEATURE (85)..(85): Xaa can be Val, Glu, Thr or Tyr
  MISC_FEATURE (93)..(93): Xaa can be Trp or Phe
  MISC_FEATURE (97)..(97): Xaa can be Lys, Glu or Gln
  MISC_FEATURE (98)..(98): Xaa can be Glu, His, Leu, Gln, Arg or Tyr
  MISC_FEATURE (100)..(100): Xaa can be any amino acid except Ala, Arg, Cys, Gln, Glu or Met
  MISC_FEATURE (102)..(102): Xaa can be any amino acid except Ala, Arg, Asn, Cys, Gln, Glu, Leu, or Met
  MISC_FEATURE (103)..(103): Xaa can be Val or Ile
  MISC_FEATURE (104)..(104): Xaa can be any amino acid except for Ala, Cys, Asn, Gln, Glu or Lys
  MISC_FEATURE (105)..(105): Xaa can be any amino acid except for Cys
  MISC_FEATURE (106)..(106): Xaa can be Lys, Ile, Leu, Pro or Thr
  MISC_FEATURE (107)..(107): Xaa can be any amino acid except Cys, Gln or Gly
  MISC_FEATURE (108)..(108): Xaa can be any amino acid except Cys
  MISC_FEATURE (109)..(109): Xaa can be any amino acid except Ala or Cys
  MISC_FEATURE (110)..(110): Xaa can be any amino acid except Asp, Cys, Gln or Lys
  MISC_FEATURE (111)..(111): Xaa can be any amino acid except Ala, Asn, Cys, Glu, Gly, Lys or Ser
  MISC_FEATURE (112)..(112): Xaa can be any amino acid except Cys or Gly
  MISC_FEATURE (113)..(113): Xaa can be Glu, Phe, His, Ile,  Leu, Met, Asn, Pro, Thr or Tyr
  MISC_FEATURE (114)..(114): Xaa can be Lys, Phe, Ile, Leu, Pro or Thr
  MISC_FEATURE (115)..(115): Xaa can be any amino acid except Ala, Cys, Gln, Glu or Lys
  MISC_FEATURE (116)..(116): Xaa can be any amino acid except Ala, Cys, Gln, Glu or Lys
  MISC_FEATURE (117)..(117): Xaa can be Ser, Glu, His or Asn
XXXXXCXXCXXXXXXXXXXXXLXXXXXKXTLMISXTPXVXCXXXXXXXXXXXXXXXWXVXXXXXXXAXTXXXXXXXXXXXXXXXXLTVLHQDXLNGXXYXCXXXXXXXXXXXXXXXX

SEQ ID NO: 48 - protein, 174 aa, Artificial Sequence
  sequence alignment of human Fc gamma receptors
GQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFMEGEPLALRCHAWKDKLVYNVLYYRNGKAFKFFHWNSNLTILKTNISHNGTYHCSGMGKHRYTSAGISQYTVKE

SEQ ID NO: 49 - protein, 176 aa, Artificial Sequence
  sequence alignment of human Fc gamma receptors
QAAAPPKAVLKLEPPWINVLQEDSVTLTCQGARSPESDSIQWFHNGNLIPTHTQPSYRFKANNNDSGEYTCQTGQTSLSDPVHLTVLSEWLVLQTPHLEFQEGETIMLRCHSWKDKPLVKVTFFQNGKSQKFSHRLDPTFSIPQANHSHSGDYHCTGNIGYTLFSSKPVTITVQVP

SEQ ID NO: 50 - protein, 175 aa, Artificial Sequence
  sequence alignment of human Fc gamma receptors
TPAAPPKAVLKLEPQWINVLQEDSVTLTCRGTHSPESDSIQWFHNGNLIPTHTQPSYRFKANNNDSGEYTCQTGQTSLSDPVHLTVLSEWLVLQTPHLEFQEGETIVLRCHSWKDKPLVKVTFFQNGKSKKFSRSDPNFSIPQANHSHSGDYHCTGNIGYTLYSSKPVTITVQAP

SEQ ID NO: 51 - protein, 174 aa, Artificial Sequence
  sequence alignment of human Fc gamma receptors
TPAAPPKAVLKLEPQWINVLQEDSVTLTCRGTHSPESDSIQWFHNGNLIPTHTQPSYRFKANNNDSGEYTCQTGQTSLSDPVHLTVLSEWLVLQTPHLEFQEGETIVLRCHSWKDKPLVKVTFFQNGKSKKFSRSDPNFSIPQANHSHSGDYHCTGNIGYTLYSSKPVTITVQA

SEQ ID NO: 52 - protein, 176 aa, Artificial Sequence
  sequence alignment of human Fc gamma receptors
RTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVFGSKNVSSETVNITITQG

SEQ ID NO: 53 - protein, 175 aa, Artificial Sequence
  sequence alignment of human Fc gamma receptors
RTEDLPKAVVFLEPQWYSVLEKDSVTLKCQGAYSPEDNSTQWFHNESLISSQASSYFIDAATVNDSGEYRCQTNLSTLSDPVQLEVHIGWLLLQAPRWVFKEEDPIHLRCHSWKNTALHKVTYLQNGKDRKYFHHNSDFHIPKATLKDSGSYFCRGLVGSKNVSSETVNITITQG

SEQ ID NO: 54 - protein, 20 aa, Artificial Sequence
  hinge region and sites of engineering
SCDKTHTCPPCPAPELLGGP

SEQ ID NO: 55 - protein, 447 aa, Artificial Sequence; identical to SEQ ID NO: 25
  Anti-CD30 enhanced ADCC

SEQ ID NO: 56 - protein, 451 aa, Artificial Sequence
  Anti-CD19 enhanced ADCC
EVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVQFNWYVDGVEVHNAKTKPREEQFNSTFRVVSVLTVVHQDWLNGKEYKCKVSNKALPAPEEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 57 - protein, 444 aa, Artificial Sequence
  Anti-CD40 enhanced ADCC
QVQLVQSGAEVKKPGASVKVSCKASGYSFTGYYIHWVRQAPGQSLEWMGRVIPNNGGTSYNQKFQGRVTISVDKSISTAYMELSSLRSEDTAVYYCAREGIYWWGHGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVQFNWYVDGVEVHNAKTKPREEQFNSTFRVVSVLTVVHQDWLNGKEYKCKVSNKALPAPEEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 58 - protein, 450 aa, Artificial Sequence
  Anti-HM1.24 enhanced ADCC
QVQLVQSGAEVKKPGASVKVSCKASGYTFTPYWMQWVRQAPGQGLEWMGSIFPGSGDTRYSQSFQGQVTISADKSISTAYMELSRLRSDDTAVYYCARGLRRGGYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVQFNWYVDGVEVHNAKTKPREEQFNSTFRVVSVLTVVHQDWLNGKEYKCKVSNKALPAPEEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 59 - protein, 451 aa, Artificial Sequence
  Anti-CD19 enhanced Fc gamma RIIb affinity
EVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSNKAFPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - protein, 451 aa, Artificial Sequence
  Anti-IgE enhanced Fc gamma RIIb affinity
QVQLQESGPGLVKPSETLSLTCAVSGYSITSGYSWNWIRQPPGKKLEWIGSITYDGSSNYNPSLKSRVTISRDTSKNQFSLKLSSVTAADTAVYYCARGSHYFGHWHFAVWGAGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSNKAFPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - protein, 453 aa, Artificial Sequence
  Anti-VEGF extended half-life
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 62 - protein, 451 aa, Artificial Sequence
  Anti-IgE extended half-life
EVQLVESGGGLVQPGGSLRLSCAVSGYSITSGYSWNWIRQAPGKGLEWVASITYDGSTNYNPSVKGRITISRDDSKNTFYLQMNSLRAEDTAVYYCARGSHYFGHWHFAVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVQFNWYVDGVEVHNAKTKPREEQFNSTFRVVSVLTVVHQDWLNGKEYKCKVSNKALPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVLHEALHSHYTQKSLSLSPGK

SEQ ID NO: 63 - protein, 450 aa, Artificial Sequence
  Anti-TNF extended half-life
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVLHEALHSHYTQKSLSLSPGK

SEQ ID NO: 64 - protein, 449 aa, Artificial Sequence
  Anti-EGFR enhanced ADCC, extended half-life
QVQLQQSGPGLVKPSQTLSLTCTVSGFSLSNYGVHWVRQAPGKGLEWMGIIWSGGSTDYSTSLKSRLTISKDTSKSQVVLTMTNMDPVDTATYYCARALTYYDYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVQFNWYVDGVEVHNAKTKPREEQFNSTFRVVSVLTVVHQDWLNGKEYKCKVSNKALPAPEEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVLHEALHSHYTQKSLSLSPGK

SEQ ID NO: 65 - protein, 449 aa, Artificial Sequence
  Anti-EGFR enhanced ADCC, extended half-life
QVQLQQSGPGLVKPSQTLSLTCTVSGFSLSNYGVHWVRQAPGKGLEWMGIIWSGGSTDYSTSLKSRLTISKDTSKSQVVLTMTNMDPVDTATYYCARALTYYDYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVQFNWYVDGVEVHNAKTKPREEQFNSTFRVVSVLTVVHQDWLNGKEYKCKVSNKALPAPEEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVLHEALHSHYTQKSLSLSPGK

SEQ ID NO: 66 - protein, 449 aa, Artificial Sequence
  Anti-EGFR enhanced ADCC, extended half-life
QVQLQQSGPGLVKPSQTLSLTCTVSGFSLSNYGVHWVRQAPGKGLEWMGIIWSGGSTDYSTSLKSRLTISKDTSKSQVVLTMTNMDPVDTATYYCARALTYYDYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVQFNWYVDGVEVHNAKTKPREEQFNSTFRVVSVLTVVHQDWLNGKEYKCKVSNKALPAPEEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHSHYTQKSLSLSPGK

SEQ ID NO: 67 - protein, 449 aa, Artificial Sequence
  Anti-EGFR ablated Fc gamma Receptor binding
QVQLQQSGPGLVKPSQTLSLTCTVSGFSLSNYGVHWVRQAPGKGLEWMGIIWSGGSTDYSTSLKSRLTISKDTSKSQVVLTMTNMDPVDTATYYCARALTYYDYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLRGPSVFLFPPKPKDTLMISRTPEVTCVVVDVSHEDPEVKFNWYVDGVEVHNAKTKPREEQYNSTYRVVSVLTVLHQDWLNGKEYKCKVSNKARPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 68 - protein, 451 aa, Artificial Sequence
  Anti-CD20 enhanced ADCC, extended half-life
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVQFNWYVDGVEVHNAKTKPREEQFNSTFRVVSVLTVVHQDWLNGKEYKCKVSNKALPAPEEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVLHEALHSHYTQKSLSLSPGK

SEQ ID NO: 69 - protein, 451 aa, Artificial Sequence
  Anti-CD20 extended half-life
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVQFNWYVDGVEVHNAKTKPREEQFNSTFRVVSVLTVVHQDWLNGKEYKCKVSNKALPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVLHEALHSHYTQKSLSLSPGK

SEQ ID NO: 70 - protein, 451 aa, Artificial Sequence
  Anti-CD20 ablated Fc gamma receptor binding
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLRGPSVFLFPPKPKDTLMISRTPEVTCVVVDVSHEDPEVKFNWYVDGVEVHNAKTKPREEQYNSTYRVVSVLTVLHQDWLNGKEYKCKVSNKARPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - protein, 217 aa, Artificial Sequence
  Anti-CD30 enhanced ADCC
EIVLTQSPDSLAVSLGERATINCKASQSVDFDGDSYLNWYQQKGQPPKVLIYAASTLQSGVPSRFSGSGSGTDFTLTINSLEAEDAATYYCQQSNEDPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 72 - protein, 219 aa, Artificial Sequence
  Anti-CD19 enhanced ADCC
DIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 73 - protein, 219 aa, Artificial Sequence
  Anti-CD40 enhanced ADCC
DVVVTQSPDSLAVSLGERATINCKSSQSLVHSNGNTFLHWYLQKPGQSPQLLIYTVSNRFSGVPDRFSGSGSGTDFTLTISSLQAEDVGVYFCSQTTHVPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 74 - protein, 214 aa, Artificial Sequence
  Anti-HM1.24 enhanced ADCC
DIVMTQSPLSLPVTLGQPASISCKASQDVNTAVAWYLQKPGKSPKLLIYSASNRYTGVPDRITGSGSGTDFTLKISRVEAEDVGVYYCQQHYSTPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 75 - protein, 218 aa, Artificial Sequence
  Anti-CD19 enhanced Fc gamma RIIb affinity
DIQLTQSPSSLSASVGDRVTITCRASQSVDYDGDSYMNWYQQKPGQPPKLLIYAASYLGSEIPARFSGSGSGTDFTLTISSLQPEDFATYYCQQSHEDPYTFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 76 - protein, 218 aa, Artificial Sequence; identical to SEQ ID NO: 75
  Anti-IgE enhanced Fc gamma RIIb affinity

SEQ ID NO: 77 - protein, 214 aa, Artificial Sequence
  Anti-VEGF extended half-life
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 78 - protein, 218 aa, Artificial Sequence
  Anti-IgE extended half-life
DIQLTQSPSSLSASVGDRVTITCRASQSVDYDGDSYMNWYQQKPGKAPKLLIYAASYLESGVPSRFSGSGSGTDFTLTISSLQPEDFATYYCQQSHEDPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 79 - protein, 214 aa, Artificial Sequence
  Anti-TNF extended half-life
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 80 - protein, 214 aa, Artificial Sequence
  Anti-EGFR enhanced ADCC, extended half-life
DIQLTQSPSSLSASVGDRVTITCRASQSISSNLHWYQQKPDQSPKLLIKYASESISGVPSRFSGSGSGTDFTLTISSLQAEDVAVYYCQQNNNWPTTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 81 - protein, 214 aa, Artificial Sequence; identical to SEQ ID NO: 80
  Anti-EGFR enhanced ADCC, extended half-life

SEQ ID NO: 82 - protein, 214 aa, Artificial Sequence; identical to SEQ ID NO: 80
  Anti-EGFR enhanced ADCC, extended half-life

SEQ ID NO: 83 - protein, 214 aa, Artificial Sequence; identical to SEQ ID NO: 80
  Anti-EGFR ablated Fc gamma receptor binding

SEQ ID NO: 84 - protein, 213 aa, Artificial Sequence; identical to SEQ ID NO: 22
  Anti-CD20 enhanced ADCC, extended half-life

SEQ ID NO: 85 - protein, 213 aa, Artificial Sequence; identical to SEQ ID NO: 22
  Anti-CD20 extended half-life

SEQ ID NO: 86 - protein, 213 aa, Artificial Sequence; identical to SEQ ID NO: 22
  Anti-CD20 ablated Fc gamma receptor binding

SEQ ID NO: 87 - protein, 330 aa, Artificial Sequence
  variants with one or more isotypic substitutions
  MISC_FEATURE (14)..(14): Xaa can be Cys or Ser
  MISC_FEATURE (16)..(16): Xaa can be Arg or Lys
  MISC_FEATURE (20)..(20): Xaa can be Glu or Gly
  MISC_FEATURE (21)..(21): Xaa can be Ser or Gly
  MISC_FEATURE (75)..(75): Xaa can be Asn or Ser
  MISC_FEATURE (76)..(76): Xaa can be Phe or Leu
  MISC_FEATURE (79)..(79): Xaa can be Gln or Lys
  MISC_FEATURE (82)..(82): Xaa can be Thr or Ile
  MISC_FEATURE (86)..(86): Xaa can be Asp or Asn
  MISC_FEATURE (97)..(97): Xaa can be Thr, Lys or Arg
  MISC_FEATURE (100)..(100): Xaa can be Arg, Pro, Leu or Ser
  MISC_FEATURE (102)..(102): Xaa can be Cys, Ser, Thr or Tyr
  MISC_FEATURE (103)..(103): Xaa can be Cys, Pro or Gly
  MISC_FEATURE (104)..(104): Xaa can be no amino acid, Asp, Leu or the sequence Leu-Gly-Asp
  MISC_FEATURE (105)..(105): Xaa can be Val, Lys, Thr or no amino acid
  MISC_FEATURE (106)..(106): Xaa can be no amino acid or Thr
  MISC_FEATURE (107)..(107): Xaa can be Glu, His or Pro
  MISC_FEATURE (108)..(108): Xaa can be no amino acid, Thr or Pro
  MISC_FEATURE (111)..(111): Xaa can be Pro, Ser, Arg, or SEQ ID NO:111
  MISC_FEATURE (116)..(116): Xaa can be Pro or Glu
  MISC_FEATURE (117)..(117): Xaa can be Val, Leu or Phe
  MISC_FEATURE (118)..(118): Xaa can be Ala or Leu
  MISC_FEATURE (119)..(119): Xaa can be no amino acid or Gly
  MISC_FEATURE (151)..(151): Xaa can be His or Gln
  MISC_FEATURE (157)..(157): Xaa can be Gln or Lys
  MISC_FEATURE (159)..(159): Xaa can be Asn or Lys
  MISC_FEATURE (179)..(179): Xaa can be Phe or Tyr
  MISC_FEATURE (183)..(183): Xaa can be Phe or Tyr
  MISC_FEATURE (192)..(192): Xaa can be Val or Leu
  MISC_FEATURE (210)..(210): Xaa can be Gly or Ala
  MISC_FEATURE (213)..(213): Xaa can be Ala or Ser
  MISC_FEATURE (214)..(214): Xaa can be Pro or Ser
  MISC_FEATURE (222)..(222): Xaa can be Thr or Ala
  MISC_FEATURE (238)..(238): Xaa can be Arg or Gln
  MISC_FEATURE (239)..(239): Xaa can be Glu or Asp
  MISC_FEATURE (241)..(241): Xaa can be Met or Leu
  MISC_FEATURE (267)..(267): Xaa can be Asn or Ser
  MISC_FEATURE (275)..(275): Xaa can be Lys or Asn
  MISC_FEATURE (280)..(280): Xaa can be Met or Val
  MISC_FEATURE (292)..(292): Xaa can be Lys or Arg
  MISC_FEATURE (302)..(302): Xaa can be Gln or Glu
  MISC_FEATURE (305)..(305): Xaa can be Val or Ile
  MISC_FEATURE (318)..(318): Xaa can be His or Arg
  MISC_FEATURE (319)..(319): Xaa can be Tyr or Phe
  MISC_FEATURE (328)..(328): Xaa can be Pro or Leu
ASTKGPSVFPLAPXSXSTSXXTAALGCLVKDYFPEPVTVSWNSGALTSGVHTFPAVLQSSGLYSLSSVVTVPSSXXGTXTYXCNVXHKPSNTKVDKXVEXKXXXXXXXCPXCPAPXXXXGPSVFLFPPKPKDTLMISRTPEVTCVVVDVSXEDPEVXFXWYVDGVEVHNAKTKPREEQXNSTXRVVSVLTVXHQDWLNGKEYKCKVSNKXLPXXIEKTISKXKGQPREPQVYTLPPSXXEXTKNQVSLTCLVKGFYPSDIAVEWESXGQPENNYXTTPPXLDSDGSFFLYSXLTVDKSRWQXGNXFSCSVMHEALHNXXTQKSLSLSXGK

SEQ ID NO: 88 - protein, 330 aa, Artificial Sequence
  IgG2 variant
  MISC_FEATURE (14)..(14): Xaa can be Cys or Ser
  MISC_FEATURE (16)..(16): Xaa can be Arg or Lys
  MISC_FEATURE (20)..(20): Xaa can be Glu or Gly
  MISC_FEATURE (21)..(21): Xaa can be Ser or Gly
  MISC_FEATURE (75)..(75): Xaa can be Asn or Ser
  MISC_FEATURE (76)..(76): Xaa can be Phe or Leu
  MISC_FEATURE (82)..(82): Xaa can be Thr or Ile
  MISC_FEATURE (86)..(86): Xaa can be Asp or Asn
  MISC_FEATURE (97)..(97): Xaa can be Thr or Lys
  MISC_FEATURE (100)..(100): Xaa can be Arg or Pro
  MISC_FEATURE (102)..(102): Xaa can be Cys or Ser
  MISC_FEATURE (104)..(104): Xaa can be no amino acid or Asp
  MISC_FEATURE (105)..(105): Xaa can be Val or Lys
  MISC_FEATURE (106)..(106): Xaa can be no amino acid or Thr
  MISC_FEATURE (107)..(107): Xaa can be Glu or His
  MISC_FEATURE (108)..(108): Xaa can be no amino acid or Thr
  MISC_FEATURE (116)..(116): Xaa can be Pro or Glu
  MISC_FEATURE (117)..(117): Xaa can be Val or Leu
  MISC_FEATURE (118)..(118): Xaa can be Ala or Leu
  MISC_FEATURE (119)..(119): Xaa can be no amino acid or Gly
  MISC_FEATURE (157)..(157): Xaa can be Gln or Lys
  MISC_FEATURE (179)..(179): Xaa can be Phe or Tyr
  MISC_FEATURE (183)..(183): Xaa can be Phe or Tyr
  MISC_FEATURE (192)..(192): Xaa can be Val or Leu
  MISC_FEATURE (210)..(210): Xaa can be Gly or Ala
  MISC_FEATURE (222)..(222): Xaa can be Thr or Ala
  MISC_FEATURE (239)..(239): Xaa can be Glu or Asp
  MISC_FEATURE (241)..(241): Xaa can be Met or Leu
  MISC_FEATURE (280)..(280): Xaa can be Met or Val
ASTKGPSVFPLAPXSXSTSXXTAALGCLVKDYFPEPVTVSWNSGALTSGVHTFPAVLQSSGLYSLSSVVTVPSSXXGTQTYXCNVXHKPSNTKVDKXVEXKXCXXXXXCPPCPAPXXXXGPSVFLFPPKPKDTLMISRTPEVTCVVVDVSHEDPEVXFNWYVDGVEVHNAKTKPREEQXNSTXRVVSVLTVXHQDWLNGKEYKCKVSNKXLPAPIEKTISKXKGQPREPQVYTLPPSRXEXTKNQVSLTCLVKGFYPSDIAVEWESNGQPENNYKTTPPXLDSDGSFFLYSKLTVDKSRWQQGNVFSCSVMHEALHNHYTQKSLSLSPGK

SEQ ID NO: 89 - protein, 330 aa, Artificial Sequence
  IgG2 variant including an amino acid sequence
  MISC_FEATURE (14)..(14): Xaa can be Cys or Ser
  MISC_FEATURE (16)..(16): Xaa can be Arg or Lys
  MISC_FEATURE (20)..(20): Xaa can be Glu or Gly
  MISC_FEATURE (21)..(21): Xaa can be Ser or Gly
  MISC_FEATURE (75)..(75): Xaa can be Asn or Ser
  MISC_FEATURE (76)..(76): Xaa can be Phe or Leu
  MISC_FEATURE (79)..(79): Xaa can be Gln or Lys
  MISC_FEATURE (82)..(82): Xaa can be Thr or Ile
  MISC_FEATURE (86)..(86): Xaa can be Asp or Asn
  MISC_FEATURE (97)..(97): Xaa can be Thr, Lys or Arg
  MISC_FEATURE (100)..(100): Xaa can be Arg, Pro, Leu or Ser
  MISC_FEATURE (102)..(102): Xaa can be Cys, Ser, Thr or Tyr
  MISC_FEATURE (103)..(103): Xaa can be Cys, Pro or Gly
  MISC_FEATURE (104)..(104): Xaa can be no amino acid, Asp, Lys, Leu, Tyr or the sequence Leu-Gly-Asp
  MISC_FEATURE (105)..(105): Xaa can be Val, Lys, Thr, no amino acid , Glu or Tyr
  MISC_FEATURE (106)..(106): Xaa can be no amino acid, Thr, Glu or Lys
  MISC_FEATURE (107)..(107): Xaa can be Glu, His, Pro or Tyr
  MISC_FEATURE (108)..(108): Xaa can be no amino acid, Thr, Pro, Glu, Lys or Trp
  MISC_FEATURE (110)..(110): Xaa can be no amino acid, Thr, Pro, Glu, Lys or Trp
  MISC_FEATURE (111)..(111): Xaa can be Pro, Ser, Arg, Glu, Gly, Lys, Tyr, or SEQ ID NO:111
  MISC_FEATURE (113)..(113): Xaa can be Pro, Ala, Glu, Gly or Tyr
  MISC_FEATURE (114)..(114): Xaa can be Ala, Glu, Gly, Lys, Pro or Tyr
  MISC_FEATURE (115)..(115): Xaa can be Pro, Glu, Gly, Lys or Tyr
  MISC_FEATURE (116)..(116): Xaa can be Pro, Glu, Ala, Asp, Phe, Gly, His, Ile, Lys, Leu, Met, Asn, Gln, Arg, Ser, Thr, Val, Trp or Tyr
  MISC_FEATURE (117)..(117): Xaa can be Val, Leu, Phe, Asp, Glu, Phe, Gly, His, Ile, Lys, Met, Asn, Pro, Gln, Arg, Ser, Thr, Trp or Tyr
  MISC_FEATURE (118)..(118): Xaa can be Ala, Leu, Asp, Phe, Gly, His, Ile, Lys, Met, Asn, Pro, Gln, Arg, Ser, Thr, Val, Trp , or Tyr
  MISC_FEATURE (119)..(119): Xaa can be no amino acid, Gly, Ala, Asp, Glu, Phe, His, Ile, Lys, Leu, Met, Asn, Pro, Gln, Arg, Ser, Thr, Val, Trp or Tyr
  MISC_FEATURE (120)..(120): Xaa can be Gly, Asp, Glu, Phe, His, Ile, Lys, Leu, Met, Asn, Pro, Gln, Arg, Ser, Thr, Val, Trp or Tyr;
  MISC_FEATURE (121)..(121): Xaa can be Pro, Asp, Glu, Phe, Gly, His, Ile, Lys, Leu, Met, Asn, Gln, Arg, Ser, Thr, Val, Trp or Tyr
  MISC_FEATURE (122)..(122): Xaa can be Ser, Asp, Glu, Phe, Gly, His, Ile, Lys, Leu, Met, Asn, Pro, Gln, Arg, Thr, Val, Trp or Tyr
  MISC_FEATURE (123)..(123): Xaa can be Val, Ala, Ile, Met or Thr
  MISC_FEATURE (124)..(124): Xaa can be Phe, Asp, Glu, Leu, Arg, Ser, Trp or Tyr
  MISC_FEATURE (126)..(126): Xaa can be Phe, Glu, His, Leu, Gln, Arg, Trp, or Tyr
  MISC_FEATURE (127)..(127): Xaa can be Pro or His
  MISC_FEATURE (128)..(128): Xaa can be Pro or Ala
  MISC_FEATURE (129)..(129): Xaa can be , Lys, Asp, Glu, His or Tyr
  MISC_FEATURE (130)..(130): Xaa can be Pro, Gly or Val
  MISC_FEATURE (132)..(132): Xaa can be Asp, His, Gln or Tyr
  MISC_FEATURE (138)..(138): Xaa can be Arg, Glu or Tyr
  MISC_FEATURE (141)..(141): Xaa can be Glu, His, Ser or Tyr
  MISC_FEATURE (143)..(143): Xaa can be Thr, Asp, Glu, His or Tyr
  MISC_FEATURE (145)..(145): Xaa can be Val, Ala, Glu, Phe, Ile or Thr
  MISC_FEATURE (146)..(146): Xaa can be Val, Ala, Ile, Met or Thr
  MISC_FEATURE (147)..(147): Xaa can be Val, Ala, Asp, Glu, Phe, Gly, His, Ile, Lys, Leu, Met, Asn, Pro, Gln, Arg, Ser, Thr, Trp, or Tyr
  MISC_FEATURE (148)..(148): Xaa can be Asp, Phe, Gly, His, Ile, Lys, Leu, Met, Pro, Gln, Arg, Ser, Thr, Val, Trp or Tyr
  MISC_FEATURE (149)..(149): Xaa can be Val, Ala, Ile, Met or Thr
  MISC_FEATURE (150)..(150): Xaa can be Ser, Asp, Glu, Phe, His, Ile, Lys, Leu, Met, Asn, Pro, Gln, Arg, Val, Trp or Tyr
  MISC_FEATURE (151)..(151): Xaa can be His, Gln, Asp, Glu, Phe, Gly, Ile, Lys, Leu, Met, Pro, Arg, Thr, Val or Trp
  MISC_FEATURE (152)..(152): Xaa can be Glu, Phe, Gly, His, Ile, Lys, Leu, Met, Asn, Pro, Arg, Ser, Thr, Val, Trp or Tyr
  MISC_FEATURE (153)..(153): Xaa can be Asp, Phe, Gly, His, Ile, Leu, Met, Pro, Gln, Arg, Ser, Thr, Trp or Tyr
  MISC_FEATURE (154)..(154): Xaa can be Pro, Ala, Asp, Glu, Phe, Gly, His, Ile, Lys, Leu, Met, Asn, Gln, Arg, Ser, Thr, Val, Trp or Tyr
  MISC_FEATURE (155)..(155): Xaa can be Glu, Asp, Phe, Gly, His, Ile, Lys, Leu, Met, Pro, Arg, Ser, Thr, Val, Trp or Tyr
  MISC_FEATURE (156)..(156): Xaa can be Val or Ile
  MISC_FEATURE (157)..(157): Xaa can be Gln, Lys, Asp, Glu, Phe, Gly, His, Ile, Leu, Met, Asn, Pro, Arg, Thr, Val, Trp or Tyr
  MISC_FEATURE (158)..(158): Xaa can be Phe, Leu or Trp
  MISC_FEATURE (159)..(159): Xaa can be Asn, Lys, Asp, Glu, Phe, Gly, His, Ile, Leu, Met, Pro, Arg, Ser, Thr, Val, Trp or Tyr
  MISC_FEATURE (161)..(161): Xaa can be Tyr, Asp, Glu, Gly, His, Ile, Lys, Leu, Met, Asn, Pro, Gln, Arg, Ser, Thr, Val or Trp
  MISC_FEATURE (163)..(163): Xaa can be Asp, Gly, Lys, Leu, Pro or Trp
  MISC_FEATURE (164)..(164): Xaa can be Gly, Asp, Glu, Lys, Asn, Pro, Gln or Tyr
  MISC_FEATURE (165)..(165): Xaa can be Val, Glu, Gly, Lys, Pro or Tyr
  MISC_FEATURE (166)..(166): Xaa can be Glu, Gly, His, Lys, Leu, Pro, Arg or Tyr
  MISC_FEATURE (167)..(167): Xaa can be Val, Asp, Glu, Leu, Asn, Gln, Thr or Tyr
  MISC_FEATURE (168)..(168): Xaa can be His, Asp, Glu, Lys, Gln, Trp or Tyr
  MISC_FEATURE (169)..(169): Xaa can be Asn, Glu, Gly, Pro or Tyr
  MISC_FEATURE (171)..(171): Xaa can be Lys, Asp, Glu or Tyr
  MISC_FEATURE (173)..(173): Xaa can be Lys, Asp, His, Leu, Asn or Trp
  MISC_FEATURE (174)..(174): Xaa can be Pro, Asp, Glu, Gly, His, Ile, Gln or Thr
  MISC_FEATURE (175)..(175): Xaa can be Arg, Asp, Glu, Thr or Tyr
  MISC_FEATURE (176)..(176): Xaa can be Glu, Phe, Gly, His, Ile, Leu, Met, Asn, Pro, Arg, Ser, Thr, Val, Trp or Tyr
  MISC_FEATURE (177)..(177): Xaa can be Glu, Phe, Gly, His, Ile, Lys, Leu, Met, Pro, Arg, Ser, Thr, Val, Trp or Tyr
  MISC_FEATURE (178)..(178): Xaa can be Gln, Asp, Glu, Phe, Gly, His, Ile, Met, Asn, Pro, Arg, Ser, Thr, Val, Trp or Tyr
  MISC_FEATURE (179)..(179): Xaa can be Phe, Tyr, Ala, Asp, Glu, Gly, Ile, Lys, Leu, Met, Asn, Gln, Arg, Ser, Thr or Val
  MISC_FEATURE (180)..(180): Xaa can be Asn, Asp, Glu, Phe, Gly, His, Ile, Lys, Leu, Met, Pro, Gln, Arg, Ser, Thr, Val, Trp or Tyr
  MISC_FEATURE (181)..(181): Xaa can be Ser, Glu, Phe, His, Ile, Lys, Met, Gln, Arg, Trp or Tyr
  MISC_FEATURE (182)..(182): Xaa can be Thr, Ala, Asp, Glu, Phe, Gly, His, Ile, Lys, Leu, Met, Asn, Pro, Gln, Arg, Ser, Val, Trp or Tyr
  MISC_FEATURE (183)..(183): Xaa can be Phe, Tyr, Ala, Asp, Glu, Gly, His, Lys, Met, Asn, Pro, Gln, Arg, Ser, Thr, Val or Trp
  MISC_FEATURE (184)..(184): Xaa can be Arg, Asp, Glu, His or Tyr
  MISC_FEATURE (185)..(185): Xaa can be Val or Ile
  MISC_FEATURE (186)..(186): Xaa can be Val, Asp, Glu or Tyr
  MISC_FEATURE (187)..(187): Xaa can be Ser, Asp, His, Leu, Asn or Thr
  MISC_FEATURE (188)..(188): Xaa can be Val, Glu, Thr or Tyr
  MISC_FEATURE (192)..(192): Xaa can be Val or Leu
  MISC_FEATURE (196)..(196): Xaa can be Trp or Phe
  MISC_FEATURE (200)..(200): Xaa can be Lys, Glu or Gln
  MISC_FEATURE (201)..(201): Xaa can be Glu, His, Leu, Gln, Arg or Tyr
  MISC_FEATURE (203)..(203): Xaa can be Lys, Asp, Phe, Gly, His, Ile, Leu, Asn, Pro, Ser, Thr, Val, Trp or Tyr
  MISC_FEATURE (205)..(205): Xaa can be Lys, Asp, Phe, Gly, His, Ile, Pro, Ser, Thr, Val, Trp or Tyr
  MISC_FEATURE (206)..(206): Xaa can be Val or Ile
  MISC_FEATURE (207)..(207): Xaa can be Ser, Asp, Phe, Gly, His, Ile, Leu, Met, Pro, Arg, Thr, Val, Trp or Tyr
  MISC_FEATURE (208)..(208): Xaa can be Asn, Ala, Asp, Glu, Phe, Gly, His, Ile, Lys, Leu, Met, Pro, Gln, Arg, Ser, Thr, Val, Trp or Tyr
  MISC_FEATURE (209)..(209): Xaa can be Lys, Ile, Leu, Pro or Thr
  MISC_FEATURE (210)..(210): Xaa can be Gly, Ala, Asp, Glu, Phe, His, Ile, Lys, Leu, Met, Asn, Pro, Arg, Thr, Val, Trp or Tyr
  MISC_FEATURE (211)..(211): Xaa can be Leu, Ala, Asp, Glu, Phe, Gly, His, Ile, Lys, Met, Asn, Pro, Gln, Arg, Ser, Thr, Val, Trp or Tyr
  MISC_FEATURE (212)..(212): Xaa can be Pro, Asp, Glu, Phe, Gly, His, Ile, Lys, Leu, Met, Asn, Gln, Arg, Ser, Thr, Val, Trp or Tyr
  MISC_FEATURE (213)..(213): Xaa can be Ala, Ser, Glu, Phe, Gly, His, Ile, Leu, Met, Asn, Pro, Arg, Thr, Val, Trp or Tyr
  MISC_FEATURE (214)..(214): Xaa can be Pro, Ser, Asp, Phe, His, Ile, Leu, Met, Gln, Arg, Thr, Val, Trp or Tyr
  MISC_FEATURE (215)..(215): Xaa can be Ile, Ala, Asp, Glu, Phe, His, Lys, Leu, Met, Asn, Pro, Gln, Arg, Ser, Thr, Val, Trp or Tyr
  MISC_FEATURE (216)..(216): Xaa can be Glu, Phe, His, Ile, Leu, Met, Pro, Thr or Tyr
  MISC_FEATURE (217)..(217): Xaa can be Lys, Phe, Ile, Pro or Thr
  MISC_FEATURE (218)..(218): Xaa can be Thr, Asp, Phe, Gly, His, Ile, Leu, Met, Asn, Pro, Arg, Ser, Val, Trp or Tyr
  MISC_FEATURE (219)..(219): Xaa can be Ile, Glu, Lys or Tyr
  MISC_FEATURE (220)..(220): Xaa can be Ser, Glu, His or Asn
  MISC_FEATURE (222)..(222): Xaa can be Thr or Ala
  MISC_FEATURE (238)..(238): Xaa can be Arg or Gln
  MISC_FEATURE (239)..(239): Xaa can be Glu or Asp
  MISC_FEATURE (241)..(241): Xaa can be Met or Leu
  MISC_FEATURE (267)..(267): Xaa can be Asn or Ser
  MISC_FEATURE (275)..(275): Xaa can be Lys or Asn
  MISC_FEATURE (280)..(280): Xaa can be Met or Val
  MISC_FEATURE (292)..(292): Xaa can be Lys or Arg
  MISC_FEATURE (302)..(302): Xaa can be Gln or Glu
  MISC_FEATURE (305)..(305): Xaa can be Val or Ile
  MISC_FEATURE (318)..(318): Xaa can be His or Arg
  MISC_FEATURE (319)..(319): Xaa can be Tyr or Phe
  MISC_FEATURE (328)..(328): Xaa can be Pro or Leu
ASTKGPSVFPLAPXSXSTSXXTAALGCLVKDYFPEPVTVSWNSGALTSGVHTFPAVLQSSGLYSLSSVVTVPSSXXGTXTYXCNVXHKPSNTKVDKXVEXKXXXXXXXCXXCXXXXXXXXXXXXLXXXXXKXTLMISXTPXVXCXXXXXXXXXXXXXXXWXVXXXXXXXAXTXXXXXXXXXXXXXXXXLTVXHQDXLNGXXYXCXXXXXXXXXXXXXXXXKXKGQPREPQVYTLPPSXXEXTKNQVSLTCLVKGFYPSDIAVEWESXGQPENNYXTTPPXLDSDGSFFLYSXLTVDKSRWQXGNXFSCSVMHEALHNXXTQKSLSLSXGK

SEQ ID NO: 90 - protein, 330 aa, Artificial Sequence
  IgG2 variant including an amino acid sequence
  MISC_FEATURE (14)..(14): Xaa can be Cys or Ser
  MISC_FEATURE (16)..(16): Xaa can be Arg or Lys
  MISC_FEATURE (20)..(20): Xaa can be Glu or Gly
  MISC_FEATURE (21)..(21): Xaa can be Ser or Gly
  MISC_FEATURE (75)..(75): Xaa can be Asn or Ser
  MISC_FEATURE (76)..(76): Xaa can be Phe or Leu
  MISC_FEATURE (79)..(79): Xaa can be Gln or Lys
  MISC_FEATURE (82)..(82): Xaa can be Thr or Ile
  MISC_FEATURE (86)..(86): Xaa can be Asp or Asn
  MISC_FEATURE (97)..(97): Xaa can be Thr, Lys or Arg
  MISC_FEATURE (100)..(100): Xaa can be Arg, Pro, Leu or Ser
  MISC_FEATURE (102)..(102): Xaa can be Cys, Ser, Thr or Tyr
  MISC_FEATURE (103)..(103): Xaa can be Cys, Pro or Gly
  MISC_FEATURE (104)..(104): Xaa can be no amino acid, Asp, Lys, Leu, or the sequence Leu-Gly-Asp
  MISC_FEATURE (105)..(105): Xaa can be Val, Lys, Thr, or no amino acid
  MISC_FEATURE (106)..(106): Xaa can be no amino acid or Thr
  MISC_FEATURE (107)..(107): Xaa can be Glu, His or Pro
  MISC_FEATURE (108)..(108): Xaa can be no amino acid, Thr or Pro
  MISC_FEATURE (110)..(110): Xaa can be Pro or Gly
  MISC_FEATURE (111)..(111): Xaa can be Pro, Ser, Arg, or SEQ ID NO:111
  MISC_FEATURE (116)..(116): Xaa can be Pro or Glu
  MISC_FEATURE (117)..(117): Xaa can be Val, Leu, Phe, Tyr or Ile
  MISC_FEATURE (118)..(118): Xaa can be Ala, Leu, Tyr, Ile or Asp
  MISC_FEATURE (119)..(119): Xaa can be no amino acid, Gly, Ser or Ala
  MISC_FEATURE (120)..(120): Xaa can be Gly or Asp
  MISC_FEATURE (122)..(122): Xaa can be Ser, Asp, Glu, Asn, Gln or Thr
  MISC_FEATURE (123)..(123): Xaa can be Val, Ile or Met
  MISC_FEATURE (129)..(129): Xaa can be Lys, His or Tyr
  MISC_FEATURE (138)..(138): Xaa can be Arg or Tyr
  MISC_FEATURE (141)..(141): Xaa can be Glu, His or Tyr
  MISC_FEATURE (143)..(143): Xaa can be Thr or His
  MISC_FEATURE (147)..(147): Xaa can be Val, Ile, Thr or Tyr
  MISC_FEATURE (150)..(150): Xaa can be Ser, Asp or Glu
  MISC_FEATURE (151)..(151): Xaa can be His, Gln, Asp or Glu
  MISC_FEATURE (154)..(154): Xaa can be Pro or Gly
  MISC_FEATURE (155)..(155): Xaa can be Glu, Tyr, His, Arg or Ile
  MISC_FEATURE (157)..(157): Xaa can be Gln, Lys or Glu
  MISC_FEATURE (159)..(159): Xaa can be Asn or Lys
  MISC_FEATURE (161)..(161): Xaa can be Tyr or Thr
  MISC_FEATURE (164)..(164): Xaa can be Gly, Asp or Glu
  MISC_FEATURE (166)..(166): Xaa can be Glu, Leu or His
  MISC_FEATURE (167)..(167): Xaa can be Val, Glu or Asp
  MISC_FEATURE (173)..(173): Xaa can be Lys or Asn
  MISC_FEATURE (176)..(176): Xaa can be Glu or Arg
  MISC_FEATURE (178)..(178): Xaa can be Gln or Glu
  MISC_FEATURE (179)..(179): Xaa can be Phe or Tyr
  MISC_FEATURE (183)..(183): Xaa can be Phe or Tyr
  MISC_FEATURE (187)..(187): Xaa can be Ser or Thr
  MISC_FEATURE (192)..(192): Xaa can be Val or Leu
  MISC_FEATURE (207)..(207): Xaa can be Ser, Gly or Ile
  MISC_FEATURE (209)..(209): Xaa can be Lys or Thr
  MISC_FEATURE (210)..(210): Xaa can be Gly, Ala or Asp
  MISC_FEATURE (211)..(211): Xaa can be Leu, Ala, Phe, Ile or Thr
  MISC_FEATURE (213)..(213): Xaa can be Ala, Ser, Leu, Tyr or Ile
  MISC_FEATURE (214)..(214): Xaa can be Pro or Ser
  MISC_FEATURE (215)..(215): Xaa can be Ile, Asp, Glu, Asn, Gln or Thr
  MISC_FEATURE (216)..(216): Xaa can be Glu or Tyr
  MISC_FEATURE (217)..(217): Xaa can be Lys, Phe, Ile or Thr
  MISC_FEATURE (222)..(222): Xaa can be Thr or Ala
  MISC_FEATURE (238)..(238): Xaa can be Arg or Gln
  MISC_FEATURE (239)..(239): Xaa can be Glu or Asp
  MISC_FEATURE (241)..(241): Xaa can be Met or Leu
  MISC_FEATURE (267)..(267): Xaa can be Asn or Ser
  MISC_FEATURE (275)..(275): Xaa can be Lys or Asn
  MISC_FEATURE (280)..(280): Xaa can be Met or Val
  MISC_FEATURE (292)..(292): Xaa can be Lys or Arg
  MISC_FEATURE (302)..(302): Xaa can be Gln or Glu
  MISC_FEATURE (305)..(305): Xaa can be Val or Ile
  MISC_FEATURE (318)..(318): Xaa can be His or Arg
  MISC_FEATURE (319)..(319): Xaa can be Tyr or Phe
  MISC_FEATURE (328)..(328): Xaa can be Pro or Leu
ASTKGPSVFPLAPXSXSTSXXTAALGCLVKDYFPEPVTVSWNSGALTSGVHTFPAVLQSSGLYSLSSVVTVPSSXXGTXTYXCNVXHKPSNTKVDKXVEXKXXXXXXXCXXCPAPXXXXXPXXFLFPPXPKDTLMISXTPXVXCVVXDVXXEDXXVXFXWXVDXVXXHNAKTXPRXEXXNSTXRVVXVLTVXHQDWLNGKEYKCKVXNXXXPXXXXXTISKXKGQPREPQVYTLPPSXXEXTKNQVSLTCLVKGFYPSDIAVEWESXGQPENNYXTTPPXLDSDGSFFLYSXLTVDKSRWQXGNXFSCSVMHEALHNXXTQKSLSLSXGK

SEQ ID NO: 91 - protein, 327 aa, Artificial Sequence
  IgG2 variant including an amino acid sequence
  MISC_FEATURE (107)..(107): Xaa can be Pro, Glu, Gly, Lys or Tyr
  MISC_FEATURE (108)..(108): Xaa can be Pro, Glu, Gly, Lys or Tyr
  MISC_FEATURE (113)..(113): Xaa can be Glu, Ala, Asp, Phe, Gly, His, Ile, Lys, Leu, Met, Asn, Gln, Arg, Ser, Thr, Val, Trp or Tyr
  MISC_FEATURE (114)..(114): Xaa can be Leu, Ala, Asp, Glu, Phe, Gly, His, Ile, Lys, Met, Asn, Pro, Gln, Arg, Ser, Thr, Val, Trp or Tyr
  MISC_FEATURE (115)..(115): Xaa can be Leu, Ala, Asp, Glu, Phe, Gly, His, Ile, Lys, Met, Asn, Pro, Gln, Arg, Ser, Thr, Val, Trp or Tyr
  MISC_FEATURE (116)..(116): Xaa can be Gly, Ala, Asp, Glu, Phe, His, Ile, Lys, Leu, Met, Asn, Pro, Gln, Arg, Ser, Thr, Val, Trp or Tyr
  MISC_FEATURE (117)..(117): Xaa can be Gly, Asp, Glu, Phe, His, Ile, Lys, Leu, Met, Asn, Pro, Gln, Arg, Ser, Thr, Val, Trp or Tyr
  MISC_FEATURE (118)..(118): Xaa can be Pro, Asp, Glu, Phe, Gly, His, Ile, Lys, Leu, Met, Asn, Gln, Arg, Ser, Thr, Val, Trp or Tyr
  MISC_FEATURE (119)..(119): Xaa can be Ser, Asp, Glu, Phe, Gly, His, Ile, Lys, Leu, Met, Asn, Pro, Gln, Arg, Thr, Val, Trp or Tyr
  MISC_FEATURE (120)..(120): Xaa can be Val, Ala, Ile, Met or Thr
  MISC_FEATURE (121)..(121): Xaa can be Phe, Asp, Glu, Leu, Arg, Ser, Trp or Tyr
  MISC_FEATURE (123)..(123): Xaa can be Phe, Glu, His, Leu, Gln, Arg, Trp or Tyr
  MISC_FEATURE (124)..(124): Xaa can be Pro or His
  MISC_FEATURE (125)..(125): Xaa can be Pro or Ala
  MISC_FEATURE (126)..(126): Xaa can be Lys, Asp, Glu, His or Tyr
  MISC_FEATURE (127)..(127): Xaa can be Pro, Gly or Val
  MISC_FEATURE (129)..(129): Xaa can be Asp, His, Gln or Tyr
  MISC_FEATURE (135)..(135): Xaa can be RE or Tyr
  MISC_FEATURE (138)..(138): Xaa can be Glu, His, Ser or Tyr
  MISC_FEATURE (140)..(140): Xaa can be Thr, Asp, Glu, His or Tyr
  MISC_FEATURE (142)..(142): Xaa can be Val, Ala, Glu, Phe, Ile or Thr
  MISC_FEATURE (143)..(143): Xaa can be Val, Ala, Ile, Met or Thr
  MISC_FEATURE (144)..(144): Xaa can be Val, Ala, Asp, Glu, Phe, Gly, His, Ile, Lys, Leu, Met, Asn, Pro, Gln, Arg, Ser, Thr, Trp or Tyr
  MISC_FEATURE (145)..(145): Xaa can be Asp, Phe, Gly, His, Ile, Lys, Leu, Met, Pro, Gln, Arg, Ser, Thr, Val, Trp or Tyr
  MISC_FEATURE (146)..(146): Xaa can be Val, Ala, Ile, Met or Thr
  MISC_FEATURE (147)..(147): Xaa can be Ser, Asp, Glu, Phe, His, Ile, Lys, Leu, Met, Asn, Pro, Gln, Arg, Val, Trp or Tyr
  MISC_FEATURE (148)..(148): Xaa can be His, Gln, Asp, Glu, Phe, Gly, Ile, Lys, Leu, Met, Pro, Arg, Thr, Val or Trp
  MISC_FEATURE (149)..(149): Xaa can be Glu, Phe, Gly, His, Ile, Lys, Leu, Met, Asn, Pro, Arg, Ser, Thr, Val, Trp or Tyr
  MISC_FEATURE (150)..(150): Xaa can be Asp, Phe, Gly, His, Ile, Leu, Met, Pro, Gln, Arg, Ser, Thr, Trp or Tyr
  MISC_FEATURE (151)..(151): Xaa can be Pro, Ala, Asp, Glu, Phe, Gly, His, Ile, Lys, Leu, Met, Asn, Gln, Arg, Ser, Thr, Val, Trp or Tyr
  MISC_FEATURE (152)..(152): Xaa can be Glu, Asp, Phe, Gly, His, Ile, Lys, Leu, Met, Pro, Arg, Ser, Thr, Val, Trp or Tyr
  MISC_FEATURE (153)..(153): Xaa can be Val or Ile
  MISC_FEATURE (154)..(154): Xaa can be Gln, Lys, Asp, Glu, Phe, Gly, His, Ile, Leu, Met, Asn, Pro, Arg, Thr, Val, Trp or Tyr
  MISC_FEATURE (155)..(155): Xaa can be Phe, Leu or Trp
  MISC_FEATURE (156)..(156): Xaa can be Asn, Lys, Asp, Glu, Phe, Gly, His, Ile, Leu, Met, Pro, Arg, Ser, Thr, Val, Trp or Tyr
  MISC_FEATURE (158)..(158): Xaa can be Tyr, Asp, Glu, Gly, His, Ile, Lys, Leu, Met, Asn, Pro, Gln, Arg, Ser, Thr, Val or Trp
  MISC_FEATURE (160)..(160): Xaa can be Asp, Gly, Lys, Leu, Pro or Trp
  MISC_FEATURE (161)..(161): Xaa can be Gly, Asp, Glu, Lys, Asn, Pro, Gln or Tyr
  MISC_FEATURE (162)..(162): Xaa can be Val, Glu, Gly, Lys, Pro or Tyr
  MISC_FEATURE (163)..(163): Xaa can be Glu, Gly, His, Lys, Leu, Pro, Arg or Tyr
  MISC_FEATURE (164)..(164): Xaa can be Val, Asp, Glu, Leu, Asn, Gln, Thr or Tyr
  MISC_FEATURE (165)..(165): Xaa can be His, Asp, Glu, Lys, Gln, Trp or Tyr
  MISC_FEATURE (166)..(166): Xaa can be Asn, Glu, Gly, Pro or Tyr
  MISC_FEATURE (168)..(168): Xaa can be Lys, Asp, Glu or Tyr
  MISC_FEATURE (170)..(170): Xaa can be Lys, Asp, His, Leu, Asn or Trp
  MISC_FEATURE (171)..(171): Xaa can be Pro, Asp, Glu, Gly, His, Ile, Gln or Thr
  MISC_FEATURE (172)..(172): Xaa can be Arg, Asp, Glu, Thr or Tyr
  MISC_FEATURE (173)..(173): Xaa can be Glu, Phe, Gly, His, Ile, Leu, Met, Asn, Pro, Arg, Ser, Thr, Val, Trp or Tyr
  MISC_FEATURE (174)..(174): Xaa can be Glu, Phe, Gly, His, Ile, Lys, Leu, Met, Pro, Arg, Ser, Thr, Val, Trp or Tyr
  MISC_FEATURE (175)..(175): Xaa can be Gln, Asp, Glu, Phe, Gly, His, Ile, Met, Asn, Pro, Arg, Ser, Thr, Val, Trp or Tyr
  MISC_FEATURE (176)..(176): Xaa can be Phe, Tyr, Ala, Asp, Glu, Gly, Ile, Lys, Leu, Met, Asn, Gln, Arg, Ser, Thr or Val
  MISC_FEATURE (177)..(177): Xaa can be Asn, Asp, Glu, Phe, Gly, His, Ile, Lys, Leu, Met, Pro, Gln, Arg, Ser, Thr, Val, Trp or Tyr
  MISC_FEATURE (178)..(178): Xaa can be Ser, Glu, Phe, His, Ile, Lys, Met, Gln, Arg, Trp or Tyr
  MISC_FEATURE (179)..(179): Xaa can be Thr, Ala, Asp, Glu, Phe, Gly, His, Ile, Lys, Leu, Met, Asn, Pro, Gln, Arg, Ser, Val, Trp or Tyr
  MISC_FEATURE (180)..(180): Xaa can be Phe, Tyr, Ala, Asp, Glu, Gly, His, Lys, Met, Asn, Pro, Gln, Arg, Ser, Thr, Val or Trp
  MISC_FEATURE (181)..(181): Xaa can be Arg, Asp, Glu, His or Tyr
  MISC_FEATURE (182)..(182): Xaa can be Val or Ile
  MISC_FEATURE (183)..(183): Xaa can be Val, Asp, Glu or Tyr
  MISC_FEATURE (184)..(184): Xaa can be Ser, Asp, His, Leu, Asn or Thr
  MISC_FEATURE (185)..(185): Xaa can be Val, Glu, Thr or Tyr
  MISC_FEATURE (189)..(189): Xaa can be Val or Leu
  MISC_FEATURE (193)..(193): Xaa can be Trp or Phe
  MISC_FEATURE (197)..(197): Xaa can be Lys, Glu or Gln
  MISC_FEATURE (198)..(198): Xaa can be Glu, His, Leu, Gln, Arg or Tyr
  MISC_FEATURE (200)..(200): Xaa can be Lys, Asp, Phe, Gly, His, Ile, Leu, Asn, Pro, Ser, Thr, Val, Trp or Tyr
  MISC_FEATURE (202)..(202): Xaa can be Lys, Asp, Phe, Gly, His, Ile, Pro, Ser, Thr, Val, Trp or Tyr
  MISC_FEATURE (203)..(203): Xaa can be Val or Ile
  MISC_FEATURE (204)..(204): Xaa can be Ser, Asp, Phe, Gly, His, Ile, Leu, Met, Pro, Arg, Thr, Val, Trp or Tyr
  MISC_FEATURE (205)..(205): Xaa can be Asn, Ala, Asp, Glu, Phe, Gly, His, Ile, Lys, Leu, Met, Pro, Gln, Arg, Ser, Thr, Val, Trp or Tyr
  MISC_FEATURE (206)..(206): Xaa can be Lys, Ile, Leu, Pro or Thr
  MISC_FEATURE (207)..(207): Xaa can be Gly, Ala, Asp, Glu, Phe, His, Ile, Lys, Leu, Met, Asn, Pro, Arg, Thr, Val, Trp or Tyr
  MISC_FEATURE (208)..(208): Xaa can be Leu, Ala, Asp, Glu, Phe, Gly, His, Ile, Lys, Met, Asn, Pro, Gln, Arg, Ser, Thr, Val, Trp or Tyr
  MISC_FEATURE (209)..(209): Xaa can be Pro, Asp, Glu, Phe, Gly, His, Ile, Lys, Leu, Met, Asn, Gln, Arg, Ser, Thr, Val, Trp or Tyr
  MISC_FEATURE (210)..(210): Xaa can be Ala, Ser, Glu, Phe, Gly, His, Ile, Leu, Met, Asn, Pro, Arg, Thr, Val, Trp or Tyr
  MISC_FEATURE (211)..(211): Xaa can be Pro, Ser, Asp, Phe, His, Ile, Leu, Met, Gln, Arg, Thr, Val, Trp or Tyr
  MISC_FEATURE (212)..(212): Xaa can be Ile, Ala, Asp, Glu, Phe, His, , Lys, Leu, Met, Asn, Pro, Gln, Arg, Ser, Thr, Val, Trp or Tyr
  MISC_FEATURE (213)..(213): Xaa can be Glu, Phe, His, Ile, Leu, Met, Pro, Thr or Tyr
  MISC_FEATURE (214)..(214): Xaa can be Lys, Phe, Ile, Pro or Thr
  MISC_FEATURE (215)..(215): Xaa can be Thr, Asp, Phe, Gly, His, Ile, Leu, Met, Asn, Pro, Arg, Ser, Val, Trp or Tyr
  MISC_FEATURE (216)..(216): Xaa can be Ile, Glu, Lys or Tyr
  MISC_FEATURE (217)..(217): Xaa can be Ser, Glu, His or Asn
  MISC_FEATURE (219)..(219): Xaa can be Thr or Ala
  MISC_FEATURE (235)..(235): Xaa can be Arg or Gln
  MISC_FEATURE (236)..(236): Xaa can be Glu or Asp
  MISC_FEATURE (238)..(238): Xaa can be Met or Leu
  MISC_FEATURE (264)..(264): Xaa can be Asn or Ser
  MISC_FEATURE (272)..(272): Xaa can be Lys or Asn
  MISC_FEATURE (277)..(277): Xaa can be Met or Val
  MISC_FEATURE (289)..(289): Xaa can be Lys or Arg
  MISC_FEATURE (299)..(299): Xaa can be Gln or Glu
  MISC_FEATURE (302)..(302): Xaa can be Val or Ile
  MISC_FEATURE (315)..(315): Xaa can be His or Arg
  MISC_FEATURE (316)..(316): Xaa can be Tyr or Phe
  MISC_FEATURE (325)..(325): Xaa can be Pro or Leu
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECXXCPAPXXXXXXXXXLXXXXXKXTLMISXTPXVXCXXXXXXXXXXXXXXXWXVXXXXXXXAXTXXXXXXXXXXXXXXXXLTVXHQDXLNGXXYXCXXXXXXXXXXXXXXXXKXKGQPREPQVYTLPPSXXEXTKNQVSLTCLVKGFYPSDIAVEWESXGQPENNYXTTPPXLDSDGSFFLYSXLTVDKSRWQXGNXFSCSVMHEALHNXXTQKSLSLSXGK

SEQ ID NO: 92 - protein, 327 aa, Artificial Sequence
  IgG2 variant including an amino acid sequence
  MISC_FEATURE (107)..(107): Xaa can be Pro, Glu, Gly, Lys or Tyr
  MISC_FEATURE (108)..(108): Xaa can be Pro, Glu, Gly, Lys or Tyr
  MISC_FEATURE (113)..(113): Xaa can be Glu, Ala, Asp, Phe, Gly, His, Ile, Lys, Leu, Met, Asn, Gln, Arg, Ser, Thr, Val, Trp or Tyr
  MISC_FEATURE (114)..(114): Xaa can be Glu, Ala, Asp, Phe, Gly, His, Ile, Lys, Leu, Met, Asn, Gln, Arg, Ser, Thr, Val, Trp or Tyr
  MISC_FEATURE (115)..(115): Xaa can be  Leu, Ala, Asp, Glu, Phe, Gly, His, Ile, Lys, Met, Asn, Pro, Gln, Arg, Ser, Thr, Val, Trp or Tyr
  MISC_FEATURE (116)..(116): Xaa can be Gly, Ala, Asp, Glu, Phe, His, Ile, Lys, Leu, Met, Asn, Pro, Gln, Arg, Ser, Thr, Val, Trp or Tyr
  MISC_FEATURE (117)..(117): Xaa can be Gly or Asp
  MISC_FEATURE (119)..(119): Xaa can be Ser, Asp, Glu, Asn, Gln or Thr
  MISC_FEATURE (120)..(120): Xaa can be Val, Ile or Met
  MISC_FEATURE (126)..(126): Xaa can be Lys, His or Tyr
  MISC_FEATURE (135)..(135): Xaa can be Arg or Tyr
  MISC_FEATURE (138)..(138): Xaa can be Glu, His or Tyr
  MISC_FEATURE (140)..(140): Xaa can be Thr or His
  MISC_FEATURE (144)..(144): Xaa can be Val, Ile, Thr or Tyr
  MISC_FEATURE (147)..(147): Xaa can be Ser, Asp or Glu
  MISC_FEATURE (148)..(148): Xaa can be His, Gln, Asp or Glu
  MISC_FEATURE (151)..(151): Xaa can be Pro or Gly
  MISC_FEATURE (152)..(152): Xaa can be Glu, Tyr, His, Arg or Ile
  MISC_FEATURE (154)..(154): Xaa can be Gln, Lys or Glu
  MISC_FEATURE (156)..(156): Xaa can be Asn or Lys
  MISC_FEATURE (158)..(158): Xaa can be Tyr or Thr
  MISC_FEATURE (161)..(161): Xaa can be Gly, Asp or Glu
  MISC_FEATURE (163)..(163): Xaa can be Glu, Leu or His
  MISC_FEATURE (164)..(164): Xaa can be Val, Glu or Asp
  MISC_FEATURE (170)..(170): Xaa can be Lys or Asn
  MISC_FEATURE (173)..(173): Xaa can be Glu or Arg
  MISC_FEATURE (175)..(175): Xaa can be Gln or Glu
  MISC_FEATURE (176)..(176): Xaa can be Phe or Tyr
  MISC_FEATURE (180)..(180): Xaa can be Phe or Tyr
  MISC_FEATURE (184)..(184): Xaa can be Ser or Thr
  MISC_FEATURE (189)..(189): Xaa can be Val or Leu
  MISC_FEATURE (204)..(204): Xaa can be Ser, Gly or Ile
  MISC_FEATURE (206)..(206): Xaa can be Lys or Thr
  MISC_FEATURE (207)..(207): Xaa can be Gly, Ala or Asp
  MISC_FEATURE (208)..(208): Xaa can be Leu, Ala, Phe, Ile or Thr
  MISC_FEATURE (210)..(210): Xaa can be Ala, Ser, Leu, Tyr or Ile
  MISC_FEATURE (211)..(211): Xaa can be Pro or Ser
  MISC_FEATURE (212)..(212): Xaa can be Ile, Asp, Glu, Asn, Gln or Thr
  MISC_FEATURE (213)..(213): Xaa can be Glu or Tyr
  MISC_FEATURE (214)..(214): Xaa can be Lys, Phe, Ile or Thr
  MISC_FEATURE (219)..(219): Xaa can be Thr or Ala
  MISC_FEATURE (235)..(235): Xaa can be Arg or Gln
  MISC_FEATURE (236)..(236): Xaa can be Glu or Asp
  MISC_FEATURE (238)..(238): Xaa can be Met or Leu
  MISC_FEATURE (264)..(264): Xaa can be Asn or Ser
  MISC_FEATURE (272)..(272): Xaa can be Lys or Asn
  MISC_FEATURE (277)..(277): Xaa can be Met or Val
  MISC_FEATURE (289)..(289): Xaa can be Lys or Arg
  MISC_FEATURE (299)..(299): Xaa can be Gln or Glu
  MISC_FEATURE (302)..(302): Xaa can be Val or Ile
  MISC_FEATURE (315)..(315): Xaa can be His or Arg
  MISC_FEATURE (316)..(316): Xaa can be Tyr or Phe
  MISC_FEATURE (325)..(325): Xaa can be Pro or Leu
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECXXCPAPXXXXXPXXFLFPPXPKDTLMISXTPXVXCVVXDVXXEDXXVXFXWXVDXVXXHNAKTXPRXEXXNSTXRVVXVLTVXHQDWLNGKEYKCKVXNXXXPXXXXXTISKXKGQPREPQVYTLPPSXXEXTKNQVSLTCLVKGFYPSDIAVEWESXGQPENNYXTTPPXLDSDGSFFLYSXLTVDKSRWQXGNXFSCSVMHEALHNXXTQKSLSLSXGK

SEQ ID NO: 93 - protein, 330 aa, Artificial Sequence
  IgG2 variant including an amino acid sequence
  MISC_FEATURE (104)..(104): Xaa can be no amino acid, Lys or Tyr
  MISC_FEATURE (105)..(105): Xaa can be Val, Glu or Tyr
  MISC_FEATURE (106)..(106): Xaa can be no amino acid, Glu or Lys
  MISC_FEATURE (107)..(107): Xaa can be Glu or Tyr
  MISC_FEATURE (108)..(108): Xaa can be no amino acid, Glu, Lys or Trp
  MISC_FEATURE (110)..(110): Xaa can be Pro, Glu, Gly, Lys or Tyr
  MISC_FEATURE (111)..(111): Xaa can be Pro, Glu, Gly, Lys or Tyr
  MISC_FEATURE (113)..(113): Xaa can be Pro, Ala, Glu, Gly or Tyr
  MISC_FEATURE (114)..(114): Xaa can be Ala, Glu, Gly, Lys, Pro or Tyr
  MISC_FEATURE (115)..(115): Xaa can be Pro, Glu, Gly, Lys or Tyr
  MISC_FEATURE (121)..(121): Xaa can be Pro, Asp, Glu, Phe, Gly, His, Ile, Lys, Leu, Met, Asn, Gln, Arg, Ser, Thr, Val, Trp or Tyr
  MISC_FEATURE (122)..(122): Xaa can be Ser, Asp, Glu, Phe, Gly, His, Ile, Lys, Leu, Met, Asn, Pro, Gln, Arg, Thr, Val, Trp or Tyr
  MISC_FEATURE (123)..(123): Xaa can be Val, Ala, Ile, Met or Thr
  MISC_FEATURE (124)..(124): Xaa can be Phe, Asp, Glu, Leu, Arg, Ser, Trp or Tyr
  MISC_FEATURE (126)..(126): Xaa can be Phe, Glu, His, Leu, Gln, Arg, Trp or Tyr
  MISC_FEATURE (127)..(127): Xaa can be Pro or His
  MISC_FEATURE (128)..(128): Xaa can be Pro or Ala
  MISC_FEATURE (129)..(129): Xaa can be Lys, Asp, Glu, His or Tyr
  MISC_FEATURE (130)..(130): Xaa can be Pro, Gly or Val
  MISC_FEATURE (132)..(132): Xaa can be Asp, His, Gln or Tyr
  MISC_FEATURE (138)..(138): Xaa can be Arg, Glu or Tyr
  MISC_FEATURE (141)..(141): Xaa can be Glu, His, Ser or Tyr
  MISC_FEATURE (143)..(143): Xaa can be Thr, Asp, Glu, His or Tyr
  MISC_FEATURE (145)..(145): Xaa can be Val, Ala, Glu, Phe, Ile or Thr
  MISC_FEATURE (146)..(146): Xaa can be Val, Ala, Ile, Met or Thr
  MISC_FEATURE (147)..(147): Xaa can be Val, Ala, Asp, Glu, Phe, Gly, His, Ile, Lys, Leu, Met, Asn, Pro, Gln, Arg, Ser, Thr, Trp or Tyr
  MISC_FEATURE (148)..(148): Xaa can be Asp, Phe, Gly, His, Ile, Lys, Leu, Met, Pro, Gln, Arg, Ser, Thr, Val, Trp or Tyr
  MISC_FEATURE (149)..(149): Xaa can be Val, Ala, Ile, Met or Thr
  MISC_FEATURE (150)..(150): Xaa can be Ser, Asp, Glu, Phe, His, Ile, Lys, Leu, Met, Asn, Pro, Gln, Arg, Val, Trp or Tyr
  MISC_FEATURE (151)..(151): Xaa can be His, Asp, Glu, Phe, Gly, Ile, Lys, Leu, Met, Pro, Arg, Thr, Val or Trp
  MISC_FEATURE (152)..(152): Xaa can be Glu, Phe, Gly, His, Ile, Lys, Leu, Met, Asn, Pro, Arg, Ser, Thr, Val, Trp or Tyr
  MISC_FEATURE (153)..(153): Xaa can be Asp, Phe, Gly, His, Ile, Leu, Met, Pro, Gln, Arg, Ser, Thr, Trp or Tyr
  MISC_FEATURE (154)..(154): Xaa can be Pro, Ala, Asp, Glu, Phe, Gly, His, Ile, Lys, Leu, Met, Asn, Gln, Arg, Ser, Thr, Val, Trp
  MISC_FEATURE (155)..(155): Xaa can be Glu, Asp, Phe, Gly, His, Ile, Lys, Leu, Met, Pro, Arg, Ser, Thr, Val, Trp or Tyr
  MISC_FEATURE (156)..(156): Xaa can be Val or Ile
  MISC_FEATURE (157)..(157): Xaa can be Gln, Asp, Glu, Phe, Gly, His, Ile, Leu, Met, Asn, Pro, Arg, Thr, Val, Trp or Tyr
  MISC_FEATURE (158)..(158): Xaa can be Phe, Leu or Trp
  MISC_FEATURE (159)..(159): Xaa can be Asn, Asp, Glu, Phe, Gly, His, Ile, Leu, Met, Pro, Arg, Ser, Thr, Val, Trp or Tyr
  MISC_FEATURE (161)..(161): Xaa can be Tyr, Asp, Glu, Gly, His, Ile, Lys, Leu, Met, Asn, Pro, Gln, Arg, Ser, Thr, Val or Trp
  MISC_FEATURE (163)..(163): Xaa can be Asp, Gly, Lys, Leu, Pro or Trp
  MISC_FEATURE (164)..(164): Xaa can be Gly, Asp, Glu, Lys, Asn, Pro, Gln or Tyr
  MISC_FEATURE (165)..(165): Xaa can be Val, Glu, Gly, Lys, Pro or Tyr
  MISC_FEATURE (166)..(166): Xaa can be Glu, Gly, His, Lys, Leu, Pro, Arg or Tyr
  MISC_FEATURE (167)..(167): Xaa can be Val, Asp, Glu, Leu, Asn, Gln, Thr or Tyr
  MISC_FEATURE (168)..(168): Xaa can be His, Asp, Glu, Lys, Gln, Trp or Tyr
  MISC_FEATURE (169)..(169): Xaa can be Asn, Glu, Gly, Pro or Tyr
  MISC_FEATURE (171)..(171): Xaa can be Lys, Asp, Glu or Tyr
  MISC_FEATURE (173)..(173): Xaa can be Lys, Asp, His, Leu, Asn or Trp
  MISC_FEATURE (174)..(174): Xaa can be Pro, Asp, Glu, Gly, His, Ile, Gln or Thr
  MISC_FEATURE (175)..(175): Xaa can be Arg, Asp, Glu, Thr or Tyr
  MISC_FEATURE (176)..(176): Xaa can be Glu, Phe, Gly, His, Ile, Leu, Met, Asn, Pro, Arg, Ser, Thr, Val, Trp or Tyr
  MISC_FEATURE (177)..(177): Xaa can be Glu, Phe, Gly, His, Ile, Lys, Leu, Met, Pro, Arg, Ser, Thr, Val, Trp or Tyr
  MISC_FEATURE (178)..(178): Xaa can be Gln, Asp, Glu, Phe, Gly, His, Ile, Met, Asn, Pro, Arg, Ser, Thr, Val, Trp or Tyr
  MISC_FEATURE (179)..(179): Xaa can be Phe, Ala, Asp, Glu, Gly, Ile, Lys, Leu, Met, Asn, Gln, Arg, Ser, Thr or Val
  MISC_FEATURE (180)..(180): Xaa can be Asn, Asp, Glu, Phe, Gly, His, Ile, Lys, Leu, Met, Pro, Gln, Arg, Ser, Thr, Val, Trp or Tyr
  MISC_FEATURE (181)..(181): Xaa can be Asn, Asp, Glu, Phe, Gly, His, Ile, Lys, Leu, Met, Pro, Gln, Arg, Ser, Thr, Val, Trp or Tyr
  MISC_FEATURE (182)..(182): Xaa can be Thr, Ala, Asp, Glu, Phe, Gly, His, Ile, Lys, Leu, Met, Asn, Pro, Gln, Arg, Ser, Val, Trp or Tyr
  MISC_FEATURE (183)..(183): Xaa can be Phe, Ala, Asp, Glu, Gly, His, Lys, Met, Asn, Pro, Gln, Arg, Ser, Thr, Val or Trp
  MISC_FEATURE (184)..(184): Xaa can be Arg, Asp, Glu, His or Tyr
  MISC_FEATURE (185)..(185): Xaa can be Val or Ile
  MISC_FEATURE (186)..(186): Xaa can be Val, Asp, Glu or Tyr
  MISC_FEATURE (187)..(187): Xaa can be Ser, Asp, His, Leu, Asn or Thr
  MISC_FEATURE (188)..(188): Xaa can be Ser, Asp, His, Leu, Asn or Thr
  MISC_FEATURE (196)..(196): Xaa can be Trp or Phe
  MISC_FEATURE (200)..(200): Xaa can be Lys, Glu or Gln
  MISC_FEATURE (201)..(201): Xaa can be Glu, His, Leu, Gln, Arg or Tyr
  MISC_FEATURE (203)..(203): Xaa can be Lys, Asp, Phe, Gly, His, Ile, Leu, Asn, Pro, Ser, Thr, Val, Trp or Tyr
  MISC_FEATURE (205)..(205): Xaa can be Lys, Asp, Phe, Gly, His, Ile, Pro, Ser, Thr, Val, Trp or Tyr
  MISC_FEATURE (206)..(206): Xaa can be Val or Ile
  MISC_FEATURE (207)..(207): Xaa can be Ser, Asp, Phe, Gly, His, Ile, Leu, Met, Pro, Arg, Thr, Val, Trp or Tyr
  MISC_FEATURE (208)..(208): Xaa can be Asn, Ala, Asp, Glu, Phe, Gly, His, Ile, Lys, Leu, Met, Pro, Gln, Arg, Ser, Thr, Val, Trp or Tyr
  MISC_FEATURE (209)..(209): Xaa can be Lys, Ile, Leu, Pro or Thr
  MISC_FEATURE (210)..(210): Xaa can be Ala, Gly, Asp, Glu, Phe, His, Ile, Lys, Leu, Met, Asn, Pro, Arg, Thr, Val, Trp or Tyr
  MISC_FEATURE (211)..(211): Xaa can be Leu, Ala, Asp, Glu, Phe, Gly, His, Ile, Lys, Met, Asn, Pro, Gln, Arg, Ser, Thr, Val, Trp or Tyr
  MISC_FEATURE (212)..(212): Xaa can be Pro, Asp, Glu, Phe, Gly, His, Ile, Lys, Leu, Met, Asn, Gln, Arg, Ser, Thr, Val, Trp or Tyr
  MISC_FEATURE (213)..(213): Xaa can be Ala, Glu, Phe, Gly, His, Ile, Leu, Met, Asn, Pro, Arg, Thr, Val, Trp or Tyr
  MISC_FEATURE (214)..(214): Xaa can be Pro, Asp, Phe, His, Ile, Leu, Met, Gln, Arg, Thr, Val, Trp or Tyr
  MISC_FEATURE (215)..(215): Xaa can be Ile, Ala, Asp, Glu, Phe, His, Lys, Leu, Met, Asn, Pro, Gln, Arg, Ser, Thr, , Val, Trp or Tyr
  MISC_FEATURE (216)..(216): Xaa can be Glu, Phe, His, Ile, Leu, Met, Pro, Thr or Tyr
  MISC_FEATURE (217)..(217): Xaa can be Lys, Phe, Ile, Pro or Thr
  MISC_FEATURE (218)..(218): Xaa can be Thr, Asp, Phe, Gly, His, Ile, Leu, Met, Asn, Pro, Arg, Ser, Val, Trp or Tyr
  MISC_FEATURE (219)..(219): Xaa can be Ile, Glu, Lys or Tyr
  MISC_FEATURE (220)..(220): Xaa can be Ser, Glu, His or Asn
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCXXXXXCXXCXXXELLGGXXXXLXXXXXKXTLMISXTPXVXCXXXXXXXXXXXXXXXWXVXXXXXXXAXTXXXXXXXXXXXXXXXXLTVVHQDXLNGXXYXCXXXXXXXXXXXXXXXXKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 94 - protein, 328 aa, Artificial Sequence
  IgG2 variant including an amino acid sequence
  MISC_FEATURE (104)..(104): Xaa can be no amino acid or Lys
  MISC_FEATURE (108)..(108): Xaa can be Pro or Gly
  MISC_FEATURE (120)..(120): Xaa can be Ser, Asp, Glu, Asn, Gln or Thr
  MISC_FEATURE (121)..(121): Xaa can be Val, Ile or Met
  MISC_FEATURE (127)..(127): Xaa can be Lys, His or Tyr
  MISC_FEATURE (136)..(136): Xaa can be Arg or Tyr
  MISC_FEATURE (139)..(139): Xaa can be Glu, His or Tyr
  MISC_FEATURE (141)..(141): Xaa can be Thr or His
  MISC_FEATURE (145)..(145): Xaa can be Val, Ile, Thr or Tyr
  MISC_FEATURE (148)..(148): Xaa can be Ser, Asp or Glu
  MISC_FEATURE (149)..(149): Xaa can be His, Asp or Glu
  MISC_FEATURE (152)..(152): Xaa can be Pro or Gly
  MISC_FEATURE (153)..(153): Xaa can be Glu, Tyr, His, Arg or Ile
  MISC_FEATURE (155)..(155): Xaa can be Gln or Glu
  MISC_FEATURE (159)..(159): Xaa can be Gln or Glu
  MISC_FEATURE (162)..(162): Xaa can be Gly, Asp or Glu
  MISC_FEATURE (164)..(164): Xaa can be Glu, Leu or His
  MISC_FEATURE (165)..(165): Xaa can be Val, Glu or Asp
  MISC_FEATURE (171)..(171): Xaa can be Lys or Asn
  MISC_FEATURE (174)..(174): Xaa can be Glu or Arg
  MISC_FEATURE (176)..(176): Xaa can be Gln or Glu
  MISC_FEATURE (185)..(185): Xaa can be Ser or Thr
  MISC_FEATURE (205)..(205): Xaa can be Ser, Gly or Ile
  MISC_FEATURE (207)..(207): Xaa can be Ser, Gly or Ile
  MISC_FEATURE (208)..(208): Xaa can be Ala, Gly or Asp
  MISC_FEATURE (209)..(209): Xaa can be Leu, Ala, Phe, Ile or Thr
  MISC_FEATURE (211)..(211): Xaa can be Ala, Leu, Tyr or Ile
  MISC_FEATURE (213)..(213): Xaa can be Ile, Asp, Glu, Asn, Gln or Thr
  MISC_FEATURE (214)..(214): Xaa can be Glu or Tyr
  MISC_FEATURE (215)..(215): Xaa can be Lys, Phe, Ile or Thr
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCXVECXPCPAPELLGGPXXFLFPPXPKDTLMISXTPXVXCVVXDVXXEDXXVXFNWXVDXVXXHNAKTXPRXEXFNSTFRVVXVLTVVHQDWLNGKEYKCKVXNXXXPXPXXXTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 95 - protein, 330 aa, Artificial Sequence
  IgG2 variant including an amino acid sequence
  MISC_FEATURE (104)..(104): Xaa can be no amino acid, Lys or Tyr
  MISC_FEATURE (105)..(105): Xaa can be Val, Glu or Tyr
  MISC_FEATURE (106)..(106): Xaa can be no amino acid, Glu or Lys
  MISC_FEATURE (107)..(107): Xaa can be Glu or Tyr
  MISC_FEATURE (108)..(108): Xaa can be no amino acid, Glu, Lys or Trp
  MISC_FEATURE (110)..(110): Xaa can be Pro, Glu, Gly, Lys or Tyr
  MISC_FEATURE (111)..(111): Xaa can be Pro, Glu, Gly, Lys or Tyr
  MISC_FEATURE (113)..(113): Xaa can be Pro, Ala, Glu, Gly or Tyr
  MISC_FEATURE (114)..(114): Xaa can be Ala, Glu, Gly, Lys, Pro or Tyr
  MISC_FEATURE (115)..(115): Xaa can be Ala, Glu, Gly, Lys, Pro or Tyr
  MISC_FEATURE (116)..(116): Xaa can be Pro, Ala, Asp, Phe, Gly, His, Ile, Lys, Leu, Met, Asn, Gln, Arg, Ser, Thr, Val, Trp or Tyr
  MISC_FEATURE (117)..(117): Xaa can be Val, Asp, Glu, Phe, Gly, His, Ile, Lys, Met, Asn, Pro, Gln, Arg, Ser, Thr, Trp or Tyr
  MISC_FEATURE (118)..(118): Xaa can be Ala, Asp, Phe, Gly, His, Ile, Lys, Met, Asn, Pro, Gln, Arg, Ser, Thr, Val, Trp or Tyr
  MISC_FEATURE (119)..(119): Xaa can be no amino acid, Ala, Asp, Glu, Phe, His, Ile, Lys, Leu, Met, Asn, Pro, Gln, Arg, Ser, Thr, Val, Trp or Tyr
  MISC_FEATURE (120)..(120): Xaa can be Gly, Asp, Glu, Phe, His, Ile, Lys, Leu, Met, Asn, Pro, Gln, Arg, Ser, Thr, Val, Trp or Tyr
  MISC_FEATURE (121)..(121): Xaa can be Pro, Asp, Glu, Phe, Gly, His, Ile, Lys, Leu, Met, Asn, Gln, Arg, Ser, Thr, Val, Trp or Tyr
  MISC_FEATURE (122)..(122): Xaa can be Ser, Asp, Glu, Phe, Gly, His, Ile, Lys, Leu, Met, Asn, Pro, Gln, Arg, Thr, Val, Trp or Tyr
  MISC_FEATURE (123)..(123): Xaa can be Val, Ala, Ile, Met or Thr
  MISC_FEATURE (124)..(124): Xaa can be Phe, Asp, Glu, Leu, Arg, Ser, Trp or Tyr
  MISC_FEATURE (126)..(126): Xaa can be Phe, Glu, His, Leu, Gln, Arg, Trp or Tyr
  MISC_FEATURE (127)..(127): Xaa can be Pro or His
  MISC_FEATURE (128)..(128): Xaa can be Pro or Ala
  MISC_FEATURE (129)..(129): Xaa can be Lys, Asp, Glu, His or Tyr
  MISC_FEATURE (130)..(130): Xaa can be Pro, Gly or Val
  MISC_FEATURE (132)..(132): Xaa can be Asp, His, Gln or Tyr
  MISC_FEATURE (138)..(138): Xaa can be Arg, Glu or Tyr
  MISC_FEATURE (141)..(141): Xaa can be Glu, His, Ser or Tyr
  MISC_FEATURE (143)..(143): Xaa can be Thr, Asp, Glu, His or Tyr
  MISC_FEATURE (145)..(145): Xaa can be Val, Ala, Glu, Phe, Ile or Thr
  MISC_FEATURE (146)..(146): Xaa can be Val, Ala, Ile, Met or Thr
  MISC_FEATURE (147)..(147): Xaa can be Val, Ala, Asp, Glu, Phe, Gly, His, Ile, Lys, Leu, Met, Asn, Pro, Gln, Arg, Ser, Thr, Trp
  MISC_FEATURE (148)..(148): Xaa can be Asp, Phe, Gly, His, Ile, Lys, Leu, Met, Pro, Gln, Arg, Ser, Thr, Val, Trp or Tyr
  MISC_FEATURE (149)..(149): Xaa can be Val, Ala, Ile, Met or Thr
  MISC_FEATURE (150)..(150): Xaa can be Ser, Asp, Glu, Phe, His, Ile, Lys, Leu, Met, Asn, Pro, Gln, Arg, Val, Trp or Tyr
  MISC_FEATURE (151)..(151): Xaa can be His, Asp, Glu, Phe, Gly, Ile, Lys, Leu, Met, Pro, Arg, Thr, Val or Trp
  MISC_FEATURE (152)..(152): Xaa can be Glu, Phe, Gly, His, Ile, Lys, Leu, Met, Asn, Pro, Arg, Ser, Thr, Val, Trp or Tyr
  MISC_FEATURE (153)..(153): Xaa can be Asp, Phe, Gly, His, Ile, Leu, Met, Pro, Gln, Arg, Ser, Thr, Trp or Tyr
  MISC_FEATURE (154)..(154): Xaa can be Pro, Ala, Asp, Glu, Phe, Gly, His, Ile, Lys, Leu, Met, Asn, Gln, Arg, Ser, Thr, Val, Trp or Tyr
  MISC_FEATURE (155)..(155): Xaa can be Glu, Asp, Phe, Gly, His, Ile, Lys, Leu, Met, Pro, Arg, Ser, Thr, Val, Trp or Tyr
  MISC_FEATURE (156)..(156): Xaa can be Val or Ile
  MISC_FEATURE (157)..(157): Xaa can be Gln, Asp, Glu, Phe, Gly, His, Ile, Leu, Met, Asn, Pro, Arg, Thr, Val, Trp or Tyr
  MISC_FEATURE (158)..(158): Xaa can be Phe, Leu or Trp
  MISC_FEATURE (159)..(159): Xaa can be Asn, Asp, Glu, Phe, Gly, His, Ile, Leu, Met, Pro, Arg, Ser, Thr, Val, Trp or Tyr
  MISC_FEATURE (161)..(161): Xaa can be Tyr, Asp, Glu, Gly, His, Ile, Lys, Leu, Met, Asn, Pro, Gln, Arg, Ser, Thr, Val or Trp
  MISC_FEATURE (163)..(163): Xaa can be Asp, Gly, Lys, Leu, Pro or Trp
  MISC_FEATURE (164)..(164): Xaa can be Gly, Asp, Glu, Lys, Asn, Pro, Gln or Tyr
  MISC_FEATURE (165)..(165): Xaa can be Val, Glu, Gly, Lys, Pro or Tyr
  MISC_FEATURE (166)..(166): Xaa can be Glu, Gly, His, Lys, Leu, Pro, Arg or Tyr
  MISC_FEATURE (167)..(167): Xaa can be Val, Asp, Glu, Leu, Asn, Gln, Thr or Tyr
  MISC_FEATURE (168)..(168): Xaa can be His, Asp, Glu, Lys, Gln, Trp or Tyr
  MISC_FEATURE (169)..(169): Xaa can be Asn, Glu, Gly, Pro or Tyr
  MISC_FEATURE (171)..(171): Xaa can be Lys, Asp, Glu or Tyr
  MISC_FEATURE (173)..(173): Xaa can be Lys, Asp, His, Leu, Asn or Trp
  MISC_FEATURE (174)..(174): Xaa can be Pro, Asp, Glu, Gly, His, Ile, Gln or Thr
  MISC_FEATURE (175)..(175): Xaa can be Pro, Asp, Glu, Gly, His, Ile, Gln or Thr
  MISC_FEATURE (176)..(176): Xaa can be Glu, Phe, Gly, His, Ile, Leu, Met, Asn, Pro, Arg, Ser, Thr, Val, Trp or Tyr
  MISC_FEATURE (177)..(177): Xaa can be Glu, Phe, Gly, His, Ile, Lys, Leu, Met, Pro, Arg, Ser, Thr, Val, Trp or Tyr;
  MISC_FEATURE (178)..(178): Xaa can be Gln, Asp, Glu, Phe, Gly, His, Ile, Met, Asn, Pro, Arg, Ser, Thr, Val, Trp or Tyr
  MISC_FEATURE (179)..(179): Xaa can be Phe, Ala, Asp, Glu, Gly, Ile, Lys, Leu, Met, Asn, Gln, Arg, Ser, Thr or Val
  MISC_FEATURE (180)..(180): Xaa can be Asn, Asp, Glu, Phe, Gly, His, Ile, Lys, Leu, Met, Pro, Gln, Arg, Ser, Thr, Val, Trp or Tyr
  MISC_FEATURE (181)..(181): Xaa can be Ser, Glu, Phe, His, Ile, Lys, Met, Gln, Arg, Trp or Tyr
  MISC_FEATURE (182)..(182): Xaa can be Thr, Ala, Asp, Glu, Phe, Gly, His, Ile, Lys, Leu, Met, Asn, Pro, Gln, Arg, Ser, Val, Trp or Tyr
  MISC_FEATURE (183)..(183): Xaa can be Phe, Ala, Asp, Glu, Gly, His, Lys, Met, Asn, Pro, Gln, Arg, Ser, Thr, Val or Trp
  MISC_FEATURE (184)..(184): Xaa can be Arg, Asp, Glu, His or Tyr
  MISC_FEATURE (185)..(185): Xaa can be Val or Ile
  MISC_FEATURE (186)..(186): Xaa can be Val, Asp, Glu or Tyr
  MISC_FEATURE (187)..(187): Xaa can be Ser, Asp, His, Leu, Asn or Thr
  MISC_FEATURE (188)..(188): Xaa can be Val, Glu, Thr or Tyr
  MISC_FEATURE (196)..(196): Xaa can be Trp or Phe
  MISC_FEATURE (200)..(200): Xaa can be Lys, Glu or Gln
  MISC_FEATURE (201)..(201): Xaa can be Glu, His, Leu, Gln, Arg or Tyr
  MISC_FEATURE (203)..(203): Xaa can be Lys, Asp, Phe, Gly, His, Ile, Leu, Asn, Pro, Ser, Thr, Val, Trp or Tyr
  MISC_FEATURE (205)..(205): Xaa can be Lys, Asp, Phe, Gly, His, Ile, Pro, Ser, Thr, Val, Trp or Tyr
  MISC_FEATURE (206)..(206): Xaa can be Lys, Asp, Phe, Gly, His, Ile, Pro, Ser, Thr, Val, Trp or Tyr
  MISC_FEATURE (207)..(207): Xaa can be Ser, Asp, Phe, Gly, His, Ile, Leu, Met, Pro, Arg, Thr, Val, Trp or Tyr
  MISC_FEATURE (208)..(208): Xaa can be Asn, Ala, Asp, Glu, Phe, Gly, His, Ile, Lys, Leu, Met, Pro, Gln, Arg, Ser, Thr, Val, Trp or Tyr
  MISC_FEATURE (209)..(209): Xaa can be Asn, Ala, Asp, Glu, Phe, Gly, His, Ile, Lys, Leu, Met, Pro, Gln, Arg, Ser, Thr, Val, Trp or Tyr
  MISC_FEATURE (210)..(210): Xaa can be Gly, Asp, Glu, Phe, His, Ile, Lys, Leu, Met, Asn, Pro, Arg, Thr, Val, Trp or Tyr
  MISC_FEATURE (211)..(211): Xaa can be Leu, Ala, Asp, Glu, Phe, Gly, His, Ile, Lys, Met, Asn, Pro, Gln, Arg, Ser, Thr, Val, Trp or Tyr
  MISC_FEATURE (212)..(212): Xaa can be Pro, Asp, Glu, Phe, Gly, His, Ile, Lys, Leu, Met, Asn, Gln, Arg, Ser, Thr, Val, Trp or Tyr
  MISC_FEATURE (213)..(213): Xaa can be Ala, Glu, Phe, Gly, His, Ile, Leu, Met, Asn, Pro, Arg, Thr, Val, Trp or Tyr
  MISC_FEATURE (214)..(214): Xaa can be Pro, Asp, Phe, His, Ile, Leu, Met, Gln, Arg, Thr, Val, Trp or Tyr
  MISC_FEATURE (215)..(215): Xaa can be Ile, Ala, Asp, Glu, Phe, His, Lys, Leu, Met, Asn, Pro, Gln, Arg, Ser, Thr, , Val, Trp or Tyr
  MISC_FEATURE (216)..(216): Xaa can be Glu, Phe, His, Ile, Leu, Met, Pro, Thr or Tyr
  MISC_FEATURE (217)..(217): Xaa can be Lys, Phe, Ile, Pro or Thr
  MISC_FEATURE (218)..(218): Xaa can be Thr, Asp, Phe, Gly, His, Ile, Leu, Met, Asn, Pro, Arg, Ser, Val, Trp or Tyr
  MISC_FEATURE (219)..(219): Xaa can be Ile, Glu, Lys or Tyr
  MISC_FEATURE (220)..(220): Xaa can be Ser, Glu, His or Asn
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCXXXXXCXXCXXXXXXXXXXXXLXXXXXKXTLMISXTPXVXCXXXXXXXXXXXXXXXWXVXXXXXXXAXTXXXXXXXXXXXXXXXXLTVVHQDXLNGXXYXCXXXXXXXXXXXXXXXXKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 96 - protein, 328 aa, Artificial Sequence
  IgG2 variant including an amino acid sequence
  MISC_FEATURE (104)..(104): Xaa can be no amino acid or Lys
  MISC_FEATURE (108)..(108): Xaa can be Pro or Gly
  MISC_FEATURE (115)..(115): Xaa can be Val, Tyr or Ile
  MISC_FEATURE (116)..(116): Xaa can be Ala, Tyr, Ile or Asp
  MISC_FEATURE (117)..(117): Xaa can be no amino acid, Ser or Ala
  MISC_FEATURE (118)..(118): Xaa can be Gly or Asp
  MISC_FEATURE (120)..(120): Xaa can be Gly or Asp
  MISC_FEATURE (121)..(121): Xaa can be Val, Ile or Met
  MISC_FEATURE (127)..(127): Xaa can be Lys, His or Tyr
  MISC_FEATURE (136)..(136): Xaa can be Arg or Tyr
  MISC_FEATURE (139)..(139): Xaa can be Glu, His or Tyr
  MISC_FEATURE (141)..(141): Xaa can be Thr or His
  MISC_FEATURE (145)..(145): Xaa can be Val, Ile, Thr or Tyr
  MISC_FEATURE (148)..(148): Xaa can be Ser, Asp or Glu
  MISC_FEATURE (149)..(149): Xaa can be His, Asp or Glu
  MISC_FEATURE (152)..(152): Xaa can be His, Asp or Glu
  MISC_FEATURE (153)..(153): Xaa can be Glu, Tyr, His, Arg or Ile
  MISC_FEATURE (155)..(155): Xaa can be Gln or Glu
  MISC_FEATURE (159)..(159): Xaa can be Tyr or Thr
  MISC_FEATURE (162)..(162): Xaa can be Gly, Asp or Glu
  MISC_FEATURE (164)..(164): Xaa can be Glu, Leu or His
  MISC_FEATURE (165)..(165): Xaa can be Val, Glu or Asp
  MISC_FEATURE (171)..(171): Xaa can be Lys or Asn
  MISC_FEATURE (174)..(174): Xaa can be Glu or Arg
  MISC_FEATURE (176)..(176): Xaa can be Gln or Glu
  MISC_FEATURE (185)..(185): Xaa can be Ser or Thr
  MISC_FEATURE (205)..(205): Xaa can be Ser, Gly or Ile
  MISC_FEATURE (207)..(207): Xaa can be Lys or Thr
  MISC_FEATURE (208)..(208): Xaa can be Gly or Asp
  MISC_FEATURE (209)..(209): Xaa can be Leu, Ala, Phe, Ile or Thr
  MISC_FEATURE (211)..(211): Xaa can be Ala, Leu, Tyr or Ile
  MISC_FEATURE (213)..(213): Xaa can be Ile, Asp, Glu, Asn, Gln or Thr
  MISC_FEATURE (214)..(214): Xaa can be Glu or Tyr
  MISC_FEATURE (215)..(215): Xaa can be Lys, Phe, Ile or Thr
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCXVECXPCPAPPXXXXPXXFLFPPXPKDTLMISXTPXVXCVVXDVXXEDXXVXFNWXVDXVXXHNAKTXPRXEXFNSTFRVVXVLTVVHQDWLNGKEYKCKVXNXXXPXPXXXTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 97 - protein, 377 aa, Artificial Sequence
  IgG3 variant includes an amino acid sequence
  MISC_FEATURE (14)..(14): Xaa can be Cys or Ser
  MISC_FEATURE (16)..(16): Xaa can be Arg or Lys
  MISC_FEATURE (82)..(82): Xaa can be Thr or Ile
  MISC_FEATURE (97)..(97): Xaa can be Arg or Lys
  MISC_FEATURE (100)..(100): Xaa can be Arg or Lys
  MISC_FEATURE (102)..(102): Xaa can be Thr or Ser
  MISC_FEATURE (103)..(103): Xaa can be Pro or Cys
  MISC_FEATURE (104)..(104): Xaa can be Asp Leu, or the sequence Leu-Gly-Asp
  MISC_FEATURE (107)..(107): Xaa can be Thr or Lys
  MISC_FEATURE (113)..(113): Xaa can be Arg, SEQ ID NO:111 or Pro
  MISC_FEATURE (204)..(204): Xaa can be Gln or Lys
  MISC_FEATURE (206)..(206): Xaa can be Lys or Asn
  MISC_FEATURE (230)..(230): Xaa can be Phe or Tyr
  MISC_FEATURE (269)..(269): Xaa can be Thr or Ala
  MISC_FEATURE (286)..(286): Xaa can be Glu or Asp
  MISC_FEATURE (288)..(288): Xaa can be Met or Leu
  MISC_FEATURE (314)..(314): Xaa can be Ser or Asn
  MISC_FEATURE (322)..(322): Xaa can be Asn or Lys
  MISC_FEATURE (327)..(327): Xaa can be Met or Val
  MISC_FEATURE (352)..(352): Xaa can be Ile or Val
  MISC_FEATURE (365)..(365): Xaa can be Arg or His
  MISC_FEATURE (366)..(366): Xaa can be Phe or Tyr
ASTKGPSVFPLAPXSXSTSGGTAALGCLVKDYFPEPVTVSWNSGALTSGVHTFPAVLQSSGLYSLSSVVTVPSSSLGTQTYXCNVNHKPSNTKVDKXVEXKXXXGDXTHTCPXCPEPKSCDTPPPCPRCPEPKSCDTPPPCPRCPEPKSCDTPPPCPRCPAPELLGGPSVFLFPPKPKDTLMISRTPEVTCVVVDVSHEDPEVXFXWYVDGVEVHNAKTKPREEQYNSTXRVVSVLTVLHQDWLNGKEYKCKVSNKALPAPIEKTISKXKGQPREPQVYTLPPSRXEXTKNQVSLTCLVKGFYPSDIAVEWESXGQPENNYXTTPPXLDSDGSFFLYSKLTVDKSRWQQGNXFSCSVMHEALHNXXTQKSLSLSPGK

SEQ ID NO: 98 - protein, 330 aa, Artificial Sequence; identical to SEQ ID NO: 89
  IgG3 variant including an amino acid sequence
  MISC_FEATURE (14)..(14): Xaa can be Cys or Ser
  MISC_FEATURE (16)..(16): Xaa can be Arg or Lys
  MISC_FEATURE (20)..(20): Xaa can be Glu or Gly
  MISC_FEATURE (21)..(21): Xaa can be Ser or Gly
  MISC_FEATURE (75)..(75): Xaa can be Asn or Ser
  MISC_FEATURE (76)..(76): Xaa can be Phe or Leu
  MISC_FEATURE (79)..(79): Xaa can be Gln or Lys
  MISC_FEATURE (82)..(82): Xaa can be Thr or Ile
  MISC_FEATURE (86)..(86): Xaa can be Asp or Asn
  MISC_FEATURE (97)..(97): Xaa can be Thr, Lys or Arg
  MISC_FEATURE (100)..(100): Xaa can be Arg, Pro, Leu or Ser
  MISC_FEATURE (102)..(102): Xaa can be Cys, Ser, Thr or Tyr
  MISC_FEATURE (103)..(103): Xaa can be Cys, Pro or Gly
  MISC_FEATURE (104)..(104): Xaa can be no amino acid, Asp, Lys, Tyr, Leu, or the sequence Leu-Gly-Asp
  MISC_FEATURE (105)..(105): Xaa can be Val, Lys, Thr, no amino acid , Glu or Tyr
  MISC_FEATURE (106)..(106): Xaa can be no amino acid, Thr, Glu or Lys
  MISC_FEATURE (107)..(107): Xaa can be Glu, His, Pro or Tyr
  MISC_FEATURE (108)..(108): Xaa can be no amino acid, Thr, Pro, Glu, Lys or Trp
  MISC_FEATURE (110)..(110): Xaa can be Pro, Glu, Gly, Lys or Tyr
  MISC_FEATURE (111)..(111): Xaa can be Pro, Ser, Glu, Gly, Lys, Tyr, Arg, or SEQ ID NO:111
  MISC_FEATURE (113)..(113): Xaa can be Pro, Ala, Glu, Gly or Tyr
  MISC_FEATURE (114)..(114): Xaa can be Ala, Glu, Gly, Lys, Pro or Tyr
  MISC_FEATURE (115)..(115): Xaa can be Ala, Glu, Gly, Lys, Pro or Tyr
  MISC_FEATURE (116)..(116): Xaa can be Pro, Glu, Ala, Asp, Phe, Gly, His, Ile, Lys, Leu, Met, Asn, Gln, Arg, Ser, Thr, Val, Trp or Tyr
  MISC_FEATURE (117)..(117): Xaa can be Val, Leu, Phe, Asp, Glu, Phe, Gly, His, Ile, Lys, Met, Asn, Pro, Gln, Arg, Ser, Thr, Trp or Tyr
  MISC_FEATURE (118)..(118): Xaa can be Ala, Leu, Asp, Phe, Gly, His, Ile, Lys, Met, Asn, Pro, Gln, Arg, Ser, Thr, Val, Trp , or Tyr
  MISC_FEATURE (119)..(119): Xaa can be no amino acid, Gly, Ala, Asp, Glu, Phe, His, Ile, Lys, Leu, Met, Asn, Pro, Gln, Arg, Ser, Thr, Val, Trp or Tyr
  MISC_FEATURE (120)..(120): Xaa can be Gly, Asp, Glu, Phe, His, Ile, Lys, Leu, Met, Asn, Pro, Gln, Arg, Ser, Thr, Val, Trp or Tyr
  MISC_FEATURE (121)..(121): Xaa can be Pro, Asp, Glu, Phe, Gly, His, Ile, Lys, Leu, Met, Asn, Gln, Arg, Ser, Thr, Val, Trp or Tyr
  MISC_FEATURE (122)..(122): Xaa can be Ser, Asp, Glu, Phe, Gly, His, Ile, Lys, Leu, Met, Asn, Pro, Gln, Arg, Thr, Val, Trp or Tyr
  MISC_FEATURE (123)..(123): Xaa can be Val, Ala, Ile, Met or Thr
  MISC_FEATURE (124)..(124): Xaa can be Phe, Asp, Glu, Leu, Arg, Ser, Trp or Tyr
  MISC_FEATURE (126)..(126): Xaa can be Phe, Glu, His, Leu, Gln, Arg, Trp or Tyr
  MISC_FEATURE (127)..(127): Xaa can be Pro or His
  MISC_FEATURE (128)..(128): Xaa can be Pro or Ala
  MISC_FEATURE (129)..(129): Xaa can be Pro or Ala
  MISC_FEATURE (130)..(130): Xaa can be Pro, Gly or Val
  MISC_FEATURE (132)..(132): Xaa can be Asp, His, Gln or Tyr
  MISC_FEATURE (138)..(138): Xaa can be Arg, Glu or Tyr
  MISC_FEATURE (141)..(141): Xaa can be Glu, His, Ser or Tyr
  MISC_FEATURE (143)..(143): Xaa can be Thr, Asp, Glu, His or Tyr
  MISC_FEATURE (145)..(145): Xaa can be Val, Ala, Glu, Phe, Ile or Thr
  MISC_FEATURE (146)..(146): Xaa can be Val, Ala, Ile, Met or Thr
  MISC_FEATURE (147)..(147): Xaa can be Val, Ala, Asp, Glu, Phe, Gly, His, Ile, Lys, Leu, Met, Asn, Pro, Gln, Arg, Ser, Thr, Trp, or Tyr
  MISC_FEATURE (148)..(148): Xaa can be Asp, Phe, Gly, His, Ile, Lys, Leu, Met, Pro, Gln, Arg, Ser, Thr, Val, Trp or Tyr
  MISC_FEATURE (149)..(149): Xaa can be Val, Ala, Ile, Met or Thr
  MISC_FEATURE (150)..(150): Xaa can be Ser, Asp, Glu, Phe, His, Ile, Lys, Leu, Met, Asn, Pro, Gln, Arg, Val, Trp or Tyr
  MISC_FEATURE (151)..(151): Xaa can be His, Gln, Asp, Glu, Phe, Gly, Ile, Lys, Leu, Met, Pro, Arg, Thr, Val or Trp
  MISC_FEATURE (152)..(152): Xaa can be Glu, Phe, Gly, His, Ile, Lys, Leu, Met, Asn, Pro, Arg, Ser, Thr, Val, Trp or Tyr
  MISC_FEATURE (153)..(153): Xaa can be Asp, Phe, Gly, His, Ile, Leu, Met, Pro, Gln, Arg, Ser, Thr, Trp or Tyr
  MISC_FEATURE (154)..(154): Xaa can be Pro, Ala, Asp, Glu, Phe, Gly, His, Ile, Lys, Leu, Met, Asn, Gln, Arg, Ser, Thr, Val, Trp or Tyr
  MISC_FEATURE (155)..(155): Xaa can be Glu, Asp, Phe, Gly, His, Ile, Lys, Leu, Met, Pro, Arg, Ser, Thr, Val, Trp or Tyr
  MISC_FEATURE (156)..(156): Xaa can be Val or Ile
  MISC_FEATURE (157)..(157): Xaa can be Gln, Lys, Asp, Glu, Phe, Gly, His, Ile, Leu, Met, Asn, Pro, Arg, Thr, Val, Trp or Tyr
  MISC_FEATURE (158)..(158): Xaa can be Phe, Leu or Trp
  MISC_FEATURE (159)..(159): Xaa can be Asn, Lys, Asp, Glu, Phe, Gly, His, Ile, Leu, Met, Pro, Arg, Ser, Thr, Val, Trp or Tyr
  MISC_FEATURE (161)..(161): Xaa can be Tyr, Asp, Glu, Gly, His, Ile, Lys, Leu, Met, Asn, Pro, Gln, Arg, Ser, Thr, Val or Trp
  MISC_FEATURE (163)..(163): Xaa can be Asp, Gly, Lys, Leu, Pro or Trp
  MISC_FEATURE (164)..(164): Xaa can be Gly, Asp, Glu, Lys, Asn, Pro, Gln or Tyr
  MISC_FEATURE (165)..(165): Xaa can be Val, Glu, Gly, Lys, Pro or Tyr
  MISC_FEATURE (166)..(166): Xaa can be Glu, Gly, His, Lys, Leu, Pro, Arg or Tyr
  MISC_FEATURE (167)..(167): Xaa can be Val, Asp, Glu, Leu, Asn, Gln, Thr or Tyr
  MISC_FEATURE (168)..(168): Xaa can be His, Asp, Glu, Lys, Gln, Trp or Tyr
  MISC_FEATURE (169)..(169): Xaa can be Asn, Glu, Gly, Pro or Tyr
  MISC_FEATURE (171)..(171): Xaa can be Lys, Asp, Glu or Tyr
  MISC_FEATURE (173)..(173): Xaa can be Lys, Asp, His, Leu, Asn or Trp
  MISC_FEATURE (174)..(174): Xaa can be Pro, Asp, Glu, Gly, His, Ile, Gln or Thr
  MISC_FEATURE (175)..(175): Xaa can be Arg, Asp, Glu, Thr or Tyr
  MISC_FEATURE (176)..(176): Xaa can be Glu, Phe, Gly, His, Ile, Leu, Met, Asn, Pro, Arg, Ser, Thr, Val, Trp or Tyr
  MISC_FEATURE (177)..(177): Xaa can be Glu, Phe, Gly, His, Ile, Lys, Leu, Met, Pro, Arg, Ser, Thr, Val, Trp or Tyr
  MISC_FEATURE (178)..(178): Xaa can be Gln, Asp, Glu, Phe, Gly, His, Ile, Met, Asn, Pro, Arg, Ser, Thr, Val, Trp or Tyr
  MISC_FEATURE (179)..(179): Xaa can be Phe, Tyr, Ala, Asp, Glu, Gly, Ile, Lys, Leu, Met, Asn, Gln, Arg, Ser, Thr or Val
  MISC_FEATURE (180)..(180): Xaa can be Asn, Asp, Glu, Phe, Gly, His, Ile, Lys, Leu, Met, Pro, Gln, Arg, Ser, Thr, Val, Trp or Tyr
  MISC_FEATURE (181)..(181): Xaa can be Ser, Glu, Phe, His, Ile, Lys, Met, Gln, Arg, Trp or Tyr
  MISC_FEATURE (182)..(182): Xaa can be Thr, Ala, Asp, Glu, Phe, Gly, His, Ile, Lys, Leu, Met, Asn, Pro, Gln, Arg, Ser, Val, Trp or Tyr
  MISC_FEATURE (183)..(183): Xaa can be Phe, Tyr, Ala, Asp, Glu, Gly, His, Lys, Met, Asn, Pro, Gln, Arg, Ser, Thr, Val or Trp
  MISC_FEATURE (184)..(184): Xaa can be Arg, Asp, Glu, His or Tyr
  MISC_FEATURE (185)..(185): Xaa can be Val or Ile
  MISC_FEATURE (186)..(186): Xaa can be Val, Asp, Glu or Tyr
  MISC_FEATURE (187)..(187): Xaa can be Ser, Asp, His, Leu, Asn or Thr
  MISC_FEATURE (188)..(188): Xaa can be Val, Glu, Thr or Tyr
  MISC_FEATURE (192)..(192): Xaa can be Val or Leu
  MISC_FEATURE (196)..(196): Xaa can be Trp or Phe
  MISC_FEATURE (200)..(200): Xaa can be Lys, Glu or Gln
  MISC_FEATURE (201)..(201): Xaa can be Glu, His, Leu, Gln, Arg or Tyr
  MISC_FEATURE (203)..(203): Xaa can be Lys, Asp, Phe, Gly, His, Ile, Leu, Asn, Pro, Ser, Thr, Val, Trp or Tyr
  MISC_FEATURE (205)..(205): Xaa can be Lys, Asp, Phe, Gly, His, Ile, Pro, Ser, Thr, Val, Trp or Tyr
  MISC_FEATURE (206)..(206): Xaa can be Val or Ile
  MISC_FEATURE (207)..(207): Xaa can be Ser, Asp, Phe, Gly, His, Ile, Leu, Met, Pro, Arg, Thr, Val, Trp or Tyr
  MISC_FEATURE (208)..(208): Xaa can be Asn, Ala, Asp, Glu, Phe, Gly, His, Ile, Lys, Leu, Met, Pro, Gln, Arg, Ser, Thr, Val, Trp or Tyr
  MISC_FEATURE (209)..(209): Xaa can be Lys, Ile, Leu, Pro or Thr
  MISC_FEATURE (210)..(210): Xaa can be Gly, Ala, Asp, Glu, Phe, His, Ile, Lys, Leu, Met, Asn, Pro, Arg, Thr, Val, Trp or Tyr
  MISC_FEATURE (211)..(211): Xaa can be Leu, Ala, Asp, Glu, Phe, Gly, His, Ile, Lys, Met, Asn, Pro, Gln, Arg, Ser, Thr, Val, Trp or Tyr
  MISC_FEATURE (212)..(212): Xaa can be Pro, Asp, Glu, Phe, Gly, His, Ile, Lys, Leu, Met, Asn, Gln, Arg, Ser, Thr, Val, Trp or Tyr
  MISC_FEATURE (213)..(213): Xaa can be Ala, Ser, Glu, Phe, Gly, His, Ile, Leu, Met, Asn, Pro, Arg, Thr, Val, Trp or Tyr
  MISC_FEATURE (214)..(214): Xaa can be Pro, Ser, Asp, Phe, His, Ile, Leu, Met, Gln, Arg, Thr, Val, Trp or Tyr
  MISC_FEATURE (215)..(215): Xaa can be Ile, Ala, Asp, Glu, Phe, His, , Lys, Leu, Met, Asn, Pro, Gln, Arg, Ser, Thr, Val, Trp or Tyr
  MISC_FEATURE (216)..(216): Xaa can be Glu, Phe, His, Ile, Leu, Met, Pro, Thr or Tyr
  MISC_FEATURE (217)..(217): Xaa can be Lys, Phe, Ile, Pro or Thr
  MISC_FEATURE (218)..(218): Xaa can be Thr, Asp, Phe, Gly, His, Ile, Leu, Met, Asn, Pro, Arg, Ser, Val, Trp or Tyr
  MISC_FEATURE (219)..(219): Xaa can be Ile, Glu, Lys or Tyr
  MISC_FEATURE (220)..(220): Xaa can be Ser, Glu, His or Asn
  MISC_FEATURE (222)..(222): Xaa can be Thr or Ala
  MISC_FEATURE (238)..(238): Xaa can be Arg or Gln
  MISC_FEATURE (239)..(239): Xaa can be Glu or Asp
  MISC_FEATURE (241)..(241): Xaa can be Met or Leu
  MISC_FEATURE (267)..(267): Xaa can be Met or Leu
  MISC_FEATURE (275)..(275): Xaa can be Lys or Asn
  MISC_FEATURE (280)..(280): Xaa can be Met or Val
  MISC_FEATURE (292)..(292): Xaa can be Lys or Arg
  MISC_FEATURE (302)..(302): Xaa can be Gln or Glu
  MISC_FEATURE (305)..(305): Xaa can be Val or Ile
  MISC_FEATURE (318)..(318): Xaa can be His or Arg
  MISC_FEATURE (319)..(319): Xaa can be Tyr or Phe
  MISC_FEATURE (328)..(328): Xaa can be Pro or Leu

SEQ ID NO: 99 - protein, 330 aa, Artificial Sequence; identical to SEQ ID NO: 90
  IgG3 variant including an amino acid sequence
  MISC_FEATURE (14)..(14): Xaa can be Cys or Ser
  MISC_FEATURE (16)..(16): Xaa can be Arg or Lys
  MISC_FEATURE (20)..(20): Xaa can be Glu or Gly
  MISC_FEATURE (21)..(21): Xaa can be Ser or Gly
  MISC_FEATURE (75)..(75): Xaa can be Asn or Ser
  MISC_FEATURE (76)..(76): Xaa can be Phe or Leu
  MISC_FEATURE (79)..(79): Xaa can be Gln or Lys
  MISC_FEATURE (82)..(82): Xaa can be Thr or Ile
  MISC_FEATURE (86)..(86): Xaa can be Asp or Asn
  MISC_FEATURE (97)..(97): Xaa can be Thr, Lys or Arg
  MISC_FEATURE (100)..(100): Xaa can be Arg, Pro, Leu or Ser
  MISC_FEATURE (102)..(102): Xaa can be Cys, Ser, Thr or Tyr
  MISC_FEATURE (103)..(103): Xaa can be Cys, Pro or Gly
  MISC_FEATURE (104)..(104): Xaa can be no amino acid, Asp, Leu, Lys, or the sequence Leu-Gly-Asp
  MISC_FEATURE (105)..(105): Xaa can be Val, Lys, Thr, or no amino acid
  MISC_FEATURE (106)..(106): Xaa can be no amino acid or Thr
  MISC_FEATURE (107)..(107): Xaa can be Glu, His or Pro
  MISC_FEATURE (108)..(108): Xaa can be no amino acid, Thr or Pro
  MISC_FEATURE (110)..(110): Xaa can be Pro or Gly
  MISC_FEATURE (111)..(111): Xaa can be Pro, Arg, Ser, or SEQ ID NO:111
  MISC_FEATURE (116)..(116): Xaa can be Pro or Glu
  MISC_FEATURE (117)..(117): Xaa can be Val, , Leu, Phe, Tyr or Ile
  MISC_FEATURE (118)..(118): Xaa can be Ala, Leu, Tyr, Ile or Asp
  MISC_FEATURE (119)..(119): Xaa can be no amino acid, Gly, Ser or Ala
  MISC_FEATURE (120)..(120): Xaa can be Gly or Asp
  MISC_FEATURE (122)..(122): Xaa can be Ser, Asp, Glu, Asn, Gln or Thr
  MISC_FEATURE (123)..(123): Xaa can be Val, Ile or Met
  MISC_FEATURE (129)..(129): Xaa can be Lys, His or Tyr
  MISC_FEATURE (138)..(138): Xaa can be Arg or Tyr
  MISC_FEATURE (141)..(141): Xaa can be Glu, His or Tyr
  MISC_FEATURE (143)..(143): Xaa can be Glu, His or Tyr
  MISC_FEATURE (147)..(147): Xaa can be Glu, His or Tyr
  MISC_FEATURE (150)..(150): Xaa can be Ser, Asp or Glu
  MISC_FEATURE (151)..(151): Xaa can be His, Gln, Asp or Glu
  MISC_FEATURE (154)..(154): Xaa can be Pro or Gly
  MISC_FEATURE (155)..(155): Xaa can be Glu, Tyr, His, Arg or Ile
  MISC_FEATURE (157)..(157): Xaa can be Gln, Lys or Glu
  MISC_FEATURE (159)..(159): Xaa can be Asn or Lys
  MISC_FEATURE (161)..(161): Xaa can be Tyr or Thr
  MISC_FEATURE (164)..(164): Xaa can be Gly, Asp or Glu
  MISC_FEATURE (166)..(166): Xaa can be Glu, Leu or His
  MISC_FEATURE (167)..(167): Xaa can be Val, Glu or Asp
  MISC_FEATURE (173)..(173): Xaa can be Lys or Asn
  MISC_FEATURE (176)..(176): Xaa can be Glu or Arg
  MISC_FEATURE (178)..(178): Xaa can be Gln or Glu
  MISC_FEATURE (179)..(179): Xaa can be Phe or Tyr
  MISC_FEATURE (183)..(183): Xaa can be Phe or Tyr
  MISC_FEATURE (187)..(187): Xaa can be Ser or Thr
  MISC_FEATURE (192)..(192): Xaa can be Val or Leu
  MISC_FEATURE (207)..(207): Xaa can be Ser, Gly or Ile
  MISC_FEATURE (209)..(209): Xaa can be Lys or Thr
  MISC_FEATURE (210)..(210): Xaa can be Gly, Ala or Asp
  MISC_FEATURE (211)..(211): Xaa can be Leu, Ala, Phe, Ile or Thr
  MISC_FEATURE (213)..(213): Xaa can be Ala, Ser, Leu, Tyr or Ile
  MISC_FEATURE (214)..(214): Xaa can be Pro or Ser
  MISC_FEATURE (215)..(215): Xaa can be Ile, Asp, Glu, Asn, Gln or Thr
  MISC_FEATURE (216)..(216): Xaa can be Glu or Tyr
  MISC_FEATURE (217)..(217): Xaa can be Lys, Phe, Ile or Thr
  MISC_FEATURE (222)..(222): Xaa can be Thr or Ala
  MISC_FEATURE (238)..(238): Xaa can be Arg or Gln
  MISC_FEATURE (239)..(239): Xaa can be Glu or Asp
  MISC_FEATURE (241)..(241): Xaa can be Met or Leu
  MISC_FEATURE (267)..(267): Xaa can be Met or Leu
  MISC_FEATURE (275)..(275): Xaa can be Lys or Asn
  MISC_FEATURE (280)..(280): Xaa can be Met or Val
  MISC_FEATURE (292)..(292): Xaa can be Lys or Arg
  MISC_FEATURE (302)..(302): Xaa can be Gln or Glu
  MISC_FEATURE (305)..(305): Xaa can be Val or Ile
  MISC_FEATURE (318)..(318): Xaa can be His or Arg
  MISC_FEATURE (319)..(319): Xaa can be Tyr or Phe
  MISC_FEATURE (328)..(328): Xaa can be Pro or Leu

SEQ ID NO: 100 - protein, 222 aa, Artificial Sequence
  IgG3 variant including an amino acid sequence
  MISC_FEATURE (2)..(2): Xaa can be Pro, Glu, Gly, Lys or Tyr
  MISC_FEATURE (3)..(3): Xaa can be Pro, Ser, Glu, Gly, Lys, Tyr, Arg, or SEQ ID NO:111
  MISC_FEATURE (5)..(5): Xaa can be Pro, Ala, Glu, Gly or Tyr
  MISC_FEATURE (6)..(6): Xaa can be Ala, Glu, Gly, Lys, Pro or Tyr
  MISC_FEATURE (7)..(7): Xaa can be Pro, Glu, Gly, Lys or Tyr
  MISC_FEATURE (8)..(8): Xaa can be Pro, Glu, Ala, Asp, Phe, Gly, His, Ile, Lys, Leu, Met, Asn, Gln, Arg, Ser, Thr, Val, Trp or Tyr
  MISC_FEATURE (9)..(9): Xaa can be Val, Leu, Phe, Asp, Glu, Phe, Gly, His, Ile, Lys, Met, Asn, Pro, Gln, Arg, Ser, Thr, Trp or Tyr
  MISC_FEATURE (10)..(10): Xaa can be Ala, Leu, Asp, Phe, Gly, His, Ile, Lys, Met, Asn, Pro, Gln, Arg, Ser, Thr, Val, Trp , or Tyr
  MISC_FEATURE (11)..(11): Xaa can be no amino acid, Gly, Ala, Asp, Glu, Phe, His, Ile, Lys, Leu, Met, Asn, Pro, Gln, Arg, Ser, Thr, Val, Trp or Tyr
  MISC_FEATURE (12)..(12): Xaa can be Gly, Asp, Glu, Phe, His, Ile, Lys, Leu, Met, Asn, Pro, Gln, Arg, Ser, Thr, Val, Trp or Tyr
  MISC_FEATURE (13)..(13): Xaa can be Pro, Asp, Glu, Phe, Gly, His, Ile, Lys, Leu, Met, Asn, Gln, Arg, Ser, Thr, Val, Trp or Tyr
  MISC_FEATURE (14)..(14): Xaa can be Ser, Asp, Glu, Phe, Gly, His, Ile, Lys, Leu, Met, Asn, Pro, Gln, Arg, Thr, Val, Trp or Tyr
  MISC_FEATURE (15)..(15): Xaa can be Val, Ala, Ile, Met or Thr
  MISC_FEATURE (16)..(16): Xaa can be Phe, Asp, Glu, Leu, Arg, Ser, Trp or Tyr
  MISC_FEATURE (18)..(18): Xaa can be Phe, Glu, His, Leu, Gln, Arg, Trp or Tyr
  MISC_FEATURE (19)..(19): Xaa can be Pro or His
  MISC_FEATURE (20)..(20): Xaa can be Pro or Ala
  MISC_FEATURE (21)..(21): Xaa can be , Lys, Asp, Glu, His or Tyr
  MISC_FEATURE (22)..(22): Xaa can be Pro, Gly or Val
  MISC_FEATURE (24)..(24): Xaa can be Asp, His, Gln or Tyr
  MISC_FEATURE (30)..(30): Xaa can be Arg, Glu or Tyr
  MISC_FEATURE (33)..(33): Xaa can be Glu, His, Ser or Tyr
  MISC_FEATURE (35)..(35): Xaa can be Thr, Asp, Glu, His or Tyr
  MISC_FEATURE (37)..(37): Xaa can be Val, Ala, Glu, Phe, Ile or Thr
  MISC_FEATURE (38)..(38): Xaa can be Val, Ala, Ile, Met or Thr
  MISC_FEATURE (39)..(39): Xaa can be Val, Ala, Asp, Glu, Phe, Gly, His, Ile, Lys, Leu, Met, Asn, Pro, Gln, Arg, Ser, Thr, Trp, or Tyr
  MISC_FEATURE (40)..(40): Xaa can be Asp, Phe, Gly, His, Ile, Lys, Leu, Met, Pro, Gln, Arg, Ser, Thr, Val, Trp or Tyr
  MISC_FEATURE (41)..(41): Xaa can be Val, Ala, Ile, Met or Thr
  MISC_FEATURE (42)..(42): Xaa can be Ser, Asp, Glu, Phe, His, Ile, Lys, Leu, Met, Asn, Pro, Gln, Arg, Val, Trp or Tyr
  MISC_FEATURE (43)..(43): Xaa can be His, Gln, Asp, Glu, Phe, Gly, Ile, Lys, Leu, Met, Pro, Arg, Thr, Val or Trp
  MISC_FEATURE (44)..(44): Xaa can be His, Gln, Asp, Glu, Phe, Gly, Ile, Lys, Leu, Met, Pro, Arg, Thr, Val or Trp
  MISC_FEATURE (45)..(45): Xaa can be Asp, Phe, Gly, His, Ile, Leu, Met, Pro, Gln, Arg, Ser, Thr, Trp or Tyr
  MISC_FEATURE (46)..(46): Xaa can be Pro, Ala, Asp, Glu, Phe, Gly, His, Ile, Lys, Leu, Met, Asn, Gln, Arg, Ser, Thr, Val, Trp or Tyr
  MISC_FEATURE (47)..(47): Xaa can be Glu, Asp, Phe, Gly, His, Ile, Lys, Leu, Met, Pro, Arg, Ser, Thr, Val, Trp or Tyr
  MISC_FEATURE (48)..(48): Xaa can be Val or Ile
  MISC_FEATURE (49)..(49): Xaa can be Gln, Lys, Asp, Glu, Phe, Gly, His, Ile, Leu, Met, Asn, Pro, Arg, Thr, Val, Trp or Tyr
  MISC_FEATURE (50)..(50): Xaa can be Phe. Leu or Trp
  MISC_FEATURE (51)..(51): Xaa can be Asn, Lys, Asp, Glu, Phe, Gly, His, Ile, Leu, Met, Pro, Arg, Ser, Thr, Val, Trp or Tyr
  MISC_FEATURE (53)..(53): Xaa can be Tyr, Asp, Glu, Gly, His, Ile, Lys, Leu, Met, Asn, Pro, Gln, Arg, Ser, Thr, Val or Trp
  MISC_FEATURE (55)..(55): Xaa can be Asp, Gly, Lys, Leu, Pro or Trp
  MISC_FEATURE (56)..(56): Xaa can be Gly, Asp, Glu, Lys, Asn, Pro, Gln or Tyr
  MISC_FEATURE (57)..(57): Xaa can be Val, Glu, Gly, Lys, Pro or Tyr
  MISC_FEATURE (58)..(58): Xaa can be Glu, Gly, His, Lys, Leu, Pro, Arg or Tyr
  MISC_FEATURE (59)..(59): Xaa can be Val, Asp, Glu, Leu, Asn, Gln, Thr or Tyr
  MISC_FEATURE (60)..(60): Xaa can be His, Asp, Glu, Lys, Gln, Trp or Tyr
  MISC_FEATURE (61)..(61): Xaa can be Asn, Glu, Gly, Pro or Tyr
  MISC_FEATURE (63)..(63): Xaa can be Lys, Asp, Glu or Tyr
  MISC_FEATURE (65)..(65): Xaa can be Lys, Asp, His, Leu, Asn or Trp
  MISC_FEATURE (66)..(66): Xaa can be Pro, Asp, Glu, Gly, His, Ile, Gln or Thr
  MISC_FEATURE (67)..(67): Xaa can be Arg, Asp, Glu, Thr or Tyr
  MISC_FEATURE (68)..(68): Xaa can be Glu, Phe, Gly, His, Ile, Leu, Met, Asn, Pro, Arg, Ser, Thr, Val, Trp or Tyr
  MISC_FEATURE (69)..(69): Xaa can be Glu, Phe, Gly, His, Ile, Lys, Leu, Met, Pro, Arg, Ser, Thr, Val, Trp or Tyr
  MISC_FEATURE (70)..(70): Xaa can be Gln, Asp, Glu, Phe, Gly, His, Ile, Met, Asn, Pro, Arg, Ser, Thr, Val, Trp or Tyr
  MISC_FEATURE (71)..(71): Xaa can be Phe, Tyr, Ala, Asp, Glu, Gly, Ile, Lys, Leu, Met, Asn, Gln, Arg, Ser, Thr or Val
  MISC_FEATURE (72)..(72): Xaa can be Asn, Asp, Glu, Phe, Gly, His, Ile, Lys, Leu, Met, Pro, Gln, Arg, Ser, Thr, Val, Trp or Tyr
  MISC_FEATURE (73)..(73): Xaa can be Ser, Glu, Phe, His, Ile, Lys, Met, Gln, Arg, Trp or Tyr
  MISC_FEATURE (74)..(74): Xaa can be Thr, Ala, Asp, Glu, Phe, Gly, His, Ile, Lys, Leu, Met, Asn, Pro, Gln, Arg, Ser, Val, Trp or Tyr
  MISC_FEATURE (75)..(75): Xaa can be Phe, Tyr, Ala, Asp, Glu, Gly, His, Lys, Met, Asn, Pro, Gln, Arg, Ser, Thr, Val or Trp
  MISC_FEATURE (76)..(76): Xaa can be Arg, Asp, Glu, His or Tyr
  MISC_FEATURE (77)..(77): Xaa can be Val or Ile
  MISC_FEATURE (78)..(78): Xaa can be Val, Asp, Glu or Tyr
  MISC_FEATURE (79)..(79): Xaa can be Ser, Asp, His, Leu, Asn or Thr
  MISC_FEATURE (80)..(80): Xaa can be Val, Glu, Thr or Tyr
  MISC_FEATURE (84)..(84): Xaa can be Val or Leu
  MISC_FEATURE (88)..(88): Xaa can be Trp or Phe
  MISC_FEATURE (92)..(92): Xaa can be Lys, Glu or Gln
  MISC_FEATURE (93)..(93): Xaa can be Glu, His, Leu, Gln, Arg or Tyr
  MISC_FEATURE (95)..(95): Xaa can be Lys, Asp, Phe, Gly, His, Ile, Leu, Asn, Pro, Ser, Thr, Val, Trp or Tyr
  MISC_FEATURE (97)..(97): Xaa can be Lys, Asp, Phe, Gly, His, Ile, Pro, Ser, Thr, Val, Trp or Tyr
  MISC_FEATURE (98)..(98): Xaa can be Val or Ile
  MISC_FEATURE (99)..(99): Xaa can be Ser, Asp, Phe, Gly, His, Ile, Leu, Met, Pro, Arg, Thr, Val, Trp or Tyr
  MISC_FEATURE (100)..(100): Xaa can be Asn, Ala, Asp, Glu, Phe, Gly, His, Ile, Lys, Leu, Met, Pro, Gln, Arg, Ser, Thr, Val, Trp or Tyr
  MISC_FEATURE (101)..(101): Xaa can be Lys, Ile, Leu, Pro or Thr
  MISC_FEATURE (102)..(102): Xaa can be Gly, Ala, Asp, Glu, Phe, His, Ile, Lys, Leu, Met, Asn, Pro, Arg, Thr, Val, Trp or Tyr
  MISC_FEATURE (103)..(103): Xaa can be Leu, Ala, Asp, Glu, Phe, Gly, His, Ile, Lys, Met, Asn, Pro, Gln, Arg, Ser, Thr, Val, Trp or Tyr
  MISC_FEATURE (104)..(104): Xaa can be Pro, Asp, Glu, Phe, Gly, His, Ile, Lys, Leu, Met, Asn, Gln, Arg, Ser, Thr, Val, Trp or Tyr
  MISC_FEATURE (105)..(105): Xaa can be Ala, Ser, Glu, Phe, Gly, His, Ile, Leu, Met, Asn, Pro, Arg, Thr, Val, Trp or Tyr
  MISC_FEATURE (106)..(106): Xaa can be Pro, Ser, Asp, Phe, His, Ile, Leu, Met, Gln, Arg, Thr, Val, Trp or Tyr
  MISC_FEATURE (107)..(107): Xaa can be Ile, Ala, Asp, Glu, Phe, His, , Lys, Leu, Met, Asn, Pro, Gln, Arg, Ser, Thr, Val, Trp or Tyr
  MISC_FEATURE (108)..(108): Xaa can be Glu, Phe, His, Ile, Leu, Met, Pro, Thr or Tyr
  MISC_FEATURE (109)..(109): Xaa can be Lys, Phe, Ile, Pro or Thr
  MISC_FEATURE (110)..(110): Xaa can be Thr, Asp, Phe, Gly, His, Ile, Leu, Met, Asn, Pro, Arg, Ser, Val, Trp or Tyr
  MISC_FEATURE (111)..(111): Xaa can be Ile, Glu, Lys or Tyr
  MISC_FEATURE (112)..(112): Xaa can be Ser, Glu, His or Asn
  MISC_FEATURE (114)..(114): Xaa can be Thr or Ala
  MISC_FEATURE (130)..(130): Xaa can be Arg or Gln
  MISC_FEATURE (131)..(131): Xaa can be Arg or Gln
  MISC_FEATURE (133)..(133): Xaa can be Met or Leu
  MISC_FEATURE (159)..(159): Xaa can be Asn or Ser
  MISC_FEATURE (167)..(167): Xaa can be Lys or Asn
  MISC_FEATURE (172)..(172): Xaa can be Met or Val
  MISC_FEATURE (184)..(184): Xaa can be Lys or Arg
  MISC_FEATURE (194)..(194): Xaa can be Gln or Glu
  MISC_FEATURE (197)..(197): Xaa can be Val or Ile
  MISC_FEATURE (210)..(210): Xaa can be Val or Ile
  MISC_FEATURE (211)..(211): Xaa can be Tyr or Phe
  MISC_FEATURE (220)..(220): Xaa can be Pro or Leu
CXXCXXXXXXXXXXXXLXXXXXKXTLMISXTPXVXCXXXXXXXXXXXXXXXWXVXXXXXXXAXTXXXXXXXXXXXXXXXXLTVXHQDXLNGXXYXCXXXXXXXXXXXXXXXXKXKGQPREPQVYTLPPSXXEXTKNQVSLTCLVKGFYPSDIAVEWESXGQPENNYXTTPPXLDSDGSFFLYSXLTVDKSRWQXGNXFSCSVMHEALHNXXTQKSLSLSXGK

SEQ ID NO: 101 - protein, 222 aa, Artificial Sequence
  IgG3 variant including an amino acid sequence
  MISC_FEATURE (2)..(2): Xaa can be Pro or Gly
  MISC_FEATURE (3)..(3): Xaa can be Pro, Arg, Ser, or SEQ ID NO:111
  MISC_FEATURE (8)..(8): Xaa can be Pro or Glu
  MISC_FEATURE (9)..(9): Xaa can be Val, Leu, Phe, Tyr or Ile
  MISC_FEATURE (10)..(10): Xaa can be Ala, Leu, Tyr, Ile or Asp
  MISC_FEATURE (11)..(11): Xaa can be no amino acid, Gly, Ser or Ala
  MISC_FEATURE (12)..(12): Xaa can be Gly or Asp
  MISC_FEATURE (14)..(14): Xaa can be Ser, Asp, Glu, Asn, Gln or Thr
  MISC_FEATURE (15)..(15): Xaa can be Val, Ile or Met
  MISC_FEATURE (21)..(21): Xaa can be Lys, His or Tyr
  MISC_FEATURE (30)..(30): Xaa can be Arg or Tyr
  MISC_FEATURE (33)..(33): Xaa can be Glu, His or Tyr
  MISC_FEATURE (35)..(35): Xaa can be Thr or His
  MISC_FEATURE (39)..(39): Xaa can be Val, Ile, Thr or Tyr
  MISC_FEATURE (42)..(42): Xaa can be Ser, Asp or Glu
  MISC_FEATURE (43)..(43): Xaa can be His, Gln, Asp or Glu
  MISC_FEATURE (46)..(46): Xaa can be Pro or Gly
  MISC_FEATURE (47)..(47): Xaa can be Glu, Tyr, His, Arg or Ile
  MISC_FEATURE (49)..(49): Xaa can be Gln, Lys or Glu
  MISC_FEATURE (51)..(51): Xaa can be Asn or Lys
  MISC_FEATURE (53)..(53): Xaa can be Tyr or Thr
  MISC_FEATURE (56)..(56): Xaa can be Gly, Asp or Glu
  MISC_FEATURE (58)..(58): Xaa can be Glu, Leu or His
  MISC_FEATURE (59)..(59): Xaa can be Val, Glu or Asp
  MISC_FEATURE (65)..(65): Xaa can be Lys or Asn
  MISC_FEATURE (68)..(68): Xaa can be Glu or Arg
  MISC_FEATURE (70)..(70): Xaa can be Gln or Glu
  MISC_FEATURE (71)..(71): Xaa can be Phe or Tyr
  MISC_FEATURE (75)..(75): Xaa can be Phe or Tyr
  MISC_FEATURE (79)..(79): Xaa can be Ser or Thr
  MISC_FEATURE (84)..(84): Xaa can be Val or Leu
  MISC_FEATURE (99)..(99): Xaa can be Val or Leu
  MISC_FEATURE (101)..(101): Xaa can be Lys or Thr
  MISC_FEATURE (102)..(102): Xaa can be Gly, Ala or Asp
  MISC_FEATURE (103)..(103): Xaa can be Leu, Ala, Phe, Ile or Thr
  MISC_FEATURE (105)..(105): Xaa can be Ala, Ser, Leu, Tyr or Ile
  MISC_FEATURE (106)..(106): Xaa can be Pro or Ser
  MISC_FEATURE (107)..(107): Xaa can be Ile, Asp, Glu, Asn, Gln or Thr
  MISC_FEATURE (108)..(108): Xaa can be Glu or Tyr
  MISC_FEATURE (109)..(109): Xaa can be Lys, Phe, Ile or Thr
  MISC_FEATURE (114)..(114): Xaa can be Thr or Ala
  MISC_FEATURE (130)..(130): Xaa can be Arg or Gln
  MISC_FEATURE (131)..(131): Xaa can be Glu or Asp
  MISC_FEATURE (133)..(133): Xaa can be Met or Leu
  MISC_FEATURE (159)..(159): Xaa can be Asn or Ser
  MISC_FEATURE (167)..(167): Xaa can be Lys or Asn
  MISC_FEATURE (172)..(172): Xaa can be Met or Val
  MISC_FEATURE (184)..(184): Xaa can be Lys or Arg
  MISC_FEATURE (194)..(194): Xaa can be Gln or Glu
  MISC_FEATURE (197)..(197): Xaa can be Val or Ile
  MISC_FEATURE (210)..(210): Xaa can be His or Arg
  MISC_FEATURE (211)..(211): Xaa can be Tyr or Phe
  MISC_FEATURE (220)..(220): Xaa can be Pro or Leu
CXXCPAPXXXXXPXXFLFPPXPKDTLMISXTPXVXCVVXDVXXEDXXVXFXWXVDXVXXHNAKTXPRXEXXNSTXRVVXVLTVXHQDWLNGKEYKCKVXNXXXPXXXXXTISKXKGQPREPQVYTLPPSXXEXTKNQVSLTCLVKGFYPSDIAVEWESXGQPENNYXTTPPXLDSDGSFFLYSXLTVDKSRWQXGNXFSCSVMHEALHNXXTQKSLSLSXGK

SEQ ID NO: 102 - protein, 377 aa, Artificial Sequence
  IgG3 variant including an amino acid sequence
  MISC_FEATURE (104)..(104): Xaa can be Leu, Lys or Tyr
  MISC_FEATURE (107)..(107): Xaa can be Thr, Glu or Tyr
  MISC_FEATURE (108)..(108): Xaa can be Thr, Glu or Lys
  MISC_FEATURE (109)..(109): Xaa can be His or Tyr
  MISC_FEATURE (110)..(110): Xaa can be Thr, Glu, Lys or Trp
  MISC_FEATURE (112)..(112): Xaa can be Pro, Glu, Gly, Lys or Tyr
  MISC_FEATURE (113)..(113): Xaa can be Arg, Glu, Gly, Lys or Tyr
  MISC_FEATURE (160)..(160): Xaa can be Pro, Ala, Glu, Gly or Tyr
  MISC_FEATURE (161)..(161): Xaa can be Ala, Glu, Gly, Lys, Pro or Tyr
  MISC_FEATURE (162)..(162): Xaa can be Ala, Glu, Gly, Lys, Pro or Tyr
  MISC_FEATURE (163)..(163): Xaa can be Glu, Ala, Asp, Phe, Gly, His, Ile, Lys, Leu, Met, Asn, Gln, Arg, Ser, Thr, Val, Trp or Tyr
  MISC_FEATURE (164)..(164): Xaa can be Leu, Asp, Glu, Phe, Gly, His, Ile, Lys, Met, Asn, Pro, Gln, Arg, Ser, Thr, Trp or Tyr
  MISC_FEATURE (165)..(165): Xaa can be Leu, Asp, Phe, Gly, His, Ile, Lys, Met, Asn, Pro, Gln, Arg, Ser, Thr, Val, Trp, or Tyr
  MISC_FEATURE (166)..(166): Xaa can be Gly, Ala, Asp, Glu, Phe, His, Ile, Lys, Leu, Met, Asn, Pro, Gln, Arg, Ser, Thr, Val, Trp or Tyr
  MISC_FEATURE (167)..(167): Xaa can be Gly, Asp, Glu, Phe, His, Ile, Lys, Leu, Met, Asn, Pro, Gln, Arg, Ser, Thr, Val, Trp or Tyr
  MISC_FEATURE (168)..(168): Xaa can be Pro, Asp, Glu, Phe, Gly, His, Ile, Lys, Leu, Met, Asn, Gln, Arg, Ser, Thr, Val, Trp or Tyr
  MISC_FEATURE (169)..(169): Xaa can be Ser, Asp, Glu, Phe, Gly, His, Ile, Lys, Leu, Met, Asn, Pro, Gln, Arg, Thr, Val, Trp or Tyr
  MISC_FEATURE (170)..(170): Xaa can be Val, Ala, Ile, Met or Thr
  MISC_FEATURE (171)..(171): Xaa can be Phe, Asp, Glu, Leu, Arg, Ser, Trp or Tyr
  MISC_FEATURE (173)..(173): Xaa can be Phe, Glu, His, Leu, Gln, Arg, Trp or Tyr
  MISC_FEATURE (174)..(174): Xaa can be Pro or His
  MISC_FEATURE (175)..(175): Xaa can be Pro or Ala
  MISC_FEATURE (176)..(176): Xaa can be Lys, Asp, Glu, His or Tyr
  MISC_FEATURE (177)..(177): Xaa can be Lys, Asp, Glu, His or Tyr
  MISC_FEATURE (179)..(179): Xaa can be Asp, His, Gln or Tyr
  MISC_FEATURE (185)..(185): Xaa can be Arg, Glu or Tyr
  MISC_FEATURE (188)..(188): Xaa can be Glu, His, Ser or Tyr
  MISC_FEATURE (190)..(190): Xaa can be Thr, Asp, Glu, His or Tyr
  MISC_FEATURE (192)..(192): Xaa can be Val, Ala, Glu, Phe, Ile or Thr
  MISC_FEATURE (193)..(193): Xaa can be Val, Ala, Ile, Met or Thr
  MISC_FEATURE (194)..(194): Xaa can be Val, Ala, Asp, Glu, Phe, Gly, His, Ile, Lys, Leu, Met, Asn, Pro, Gln, Arg, Ser, Thr, Trp or Tyr
  MISC_FEATURE (195)..(195): Xaa can be Asp, Phe, Gly, His, Ile, Lys, Leu, Met, Pro, Gln, Arg, Ser, Thr, Val, Trp or Tyr
  MISC_FEATURE (196)..(196): Xaa can be Val, Ala, Ile, Met or Thr
  MISC_FEATURE (197)..(197): Xaa can be Ser, Asp, Glu, Phe, His, Ile, Lys, Leu, Met, Asn, Pro, Gln, Arg, Val, Trp or Tyr
  MISC_FEATURE (198)..(198): Xaa can be His, Asp, Glu, Phe, Gly, Ile, Lys, Leu, Met, Pro, Arg, Thr, Val or Trp
  MISC_FEATURE (199)..(199): Xaa can be Glu, Phe, Gly, His, Ile, Lys, Leu, Met, Asn, Pro, Arg, Ser, Thr, Val, Trp or Tyr
  MISC_FEATURE (200)..(200): Xaa can be Asp, Phe, Gly, His, Ile, Leu, Met, Pro, Gln, Arg, Ser, Thr, Trp or Tyr
  MISC_FEATURE (201)..(201): Xaa can be Pro, Ala, Asp, Glu, Phe, Gly, His, Ile, Lys, Leu, Met, Asn, Gln, Arg, Ser, Thr, Val, Trp or Tyr
  MISC_FEATURE (202)..(202): Xaa can be Glu, Asp, Phe, Gly, His, Ile, Lys, Leu, Met, Pro, Arg, Ser, Thr, Val, Trp or Tyr
  MISC_FEATURE (203)..(203): Xaa can be Val or Ile
  MISC_FEATURE (204)..(204): Xaa can be Gln, Asp, Glu, Phe, Gly, His, Ile, Leu, Met, Asn, Pro, Arg, Thr, Val, Trp or Tyr
  MISC_FEATURE (205)..(205): Xaa can be Phe, Leu or Trp
  MISC_FEATURE (206)..(206): Xaa can be Lys, Asp, Glu, Phe, Gly, His, Ile, Leu, Met, Pro, Arg, Ser, Thr, Val, Trp or Tyr
  MISC_FEATURE (208)..(208): Xaa can be Tyr, Asp, Glu, Gly, His, Ile, Lys, Leu, Met, Asn, Pro, Gln, Arg, Ser, Thr, Val or Trp
  MISC_FEATURE (210)..(210): Xaa can be Asp, Gly, Lys, Leu, Pro or Trp
  MISC_FEATURE (211)..(211): Xaa can be Gly, Asp, Glu, Lys, Asn, Pro, Gln or Tyr
  MISC_FEATURE (212)..(212): Xaa can be Val, Glu, Gly, Lys, Pro or Tyr
  MISC_FEATURE (213)..(213): Xaa can be Glu, Gly, His, Lys, Leu, Pro, Arg or Tyr
  MISC_FEATURE (214)..(214): Xaa can be Val, Asp, Glu, Leu, Asn, Gln, Thr or Tyr
  MISC_FEATURE (215)..(215): Xaa can be His, Asp, Glu, Lys, Gln, Trp or Tyr
  MISC_FEATURE (216)..(216): Xaa can be Asn, Glu, Gly, Pro or Tyr
  MISC_FEATURE (218)..(218): Xaa can be Lys, Asp, Glu or Tyr
  MISC_FEATURE (220)..(220): Xaa can be Lys, Asp, His, Leu, Asn or Trp
  MISC_FEATURE (221)..(221): Xaa can be Pro, Asp, Glu, Gly, His, Ile, Gln or Thr
  MISC_FEATURE (222)..(222): Xaa can be Arg, Asp, Glu, Thr or Tyr
  MISC_FEATURE (223)..(223): Xaa can be Glu, Phe, Gly, His, Ile, Leu, Met, Asn, Pro, Arg, Ser, Thr, Val, Trp or Tyr
  MISC_FEATURE (224)..(224): Xaa can be Glu, Phe, Gly, His, Ile, Lys, Leu, Met, Pro, Arg, Ser, Thr, Val, Trp or Tyr
  MISC_FEATURE (225)..(225): Xaa can be Gln, Asp, Glu, Phe, Gly, His, Ile, Met, Asn, Pro, Arg, Ser, Thr, Val, Trp or Tyr
  MISC_FEATURE (226)..(226): Xaa can be Tyr, Ala, Asp, Glu, Gly, Ile, Lys, Leu, Met, Asn, Gln, Arg, Ser, Thr or Val
  MISC_FEATURE (227)..(227): Xaa can be Asn, Asp, Glu, Phe, Gly, His, Ile, Lys, Leu, Met, Pro, Gln, Arg, Ser, Thr, Val, Trp or Tyr
  MISC_FEATURE (228)..(228): Xaa can be Ser, Glu, Phe, His, Ile, Lys, Met, Gln, Arg, Trp or Tyr
  MISC_FEATURE (229)..(229): Xaa can be Thr, Ala, Asp, Glu, Phe, Gly, His, Ile, Lys, Leu, Met, Asn, Pro, Gln, Arg, Ser, Val, Trp or Tyr
  MISC_FEATURE (230)..(230): Xaa can be Phe, Ala, Asp, Glu, Gly, His, Lys, Met, Asn, Pro, Gln, Arg, Ser, Thr, Val or Trp
  MISC_FEATURE (231)..(231): Xaa can be Arg, Asp, Glu, His or Tyr
  MISC_FEATURE (232)..(232): Xaa can be Arg, Asp, Glu, His or Tyr
  MISC_FEATURE (233)..(233): Xaa can be Val, Asp, Glu or Tyr
  MISC_FEATURE (234)..(234): Xaa can be Ser, Asp, His, Leu, Asn or Thr
  MISC_FEATURE (235)..(235): Xaa can be Val, Glu, Thr or Tyr
  MISC_FEATURE (243)..(243): Xaa can be Trp or Phe
  MISC_FEATURE (247)..(247): Xaa can be Lys, Glu or Gln
  MISC_FEATURE (248)..(248): Xaa can be Glu, His, Leu, Gln, Arg or Tyr
  MISC_FEATURE (250)..(250): Xaa can be Lys, Asp, Phe, Gly, His, Ile, Leu, Asn, Pro, Ser, Thr, Val, Trp or Tyr
  MISC_FEATURE (252)..(252): Xaa can be Lys, Asp, Phe, Gly, His, Ile, Pro, Ser, Thr, Val, Trp or Tyr
  MISC_FEATURE (253)..(253): Xaa can be Val or Ile
  MISC_FEATURE (254)..(254): Xaa can be Ser, Asp, Phe, Gly, His, Ile, Leu, Met, Pro, Arg, Thr, Val, Trp or Tyr
  MISC_FEATURE (255)..(255): Xaa can be Asn, Ala, Asp, Glu, Phe, Gly, His, Ile, Lys, Leu, Met, Pro, Gln, Arg, Ser, Thr, Val, Trp or Tyr
  MISC_FEATURE (256)..(256): Xaa can be Lys, Ile, Leu, Pro or Thr
  MISC_FEATURE (257)..(257): Xaa can be Ala, Asp, Glu, Phe, His, Ile, Lys, Leu, Met, Asn, Pro, Arg, Thr, Val, Trp or Tyr
  MISC_FEATURE (258)..(258): Xaa can be Leu, Ala, Asp, Glu, Phe, Gly, His, Ile, Lys, Met, Asn, Pro, Gln, Arg, Ser, Thr, Val, Trp or Tyr
  MISC_FEATURE (259)..(259): Xaa can be Pro, Asp, Glu, Phe, Gly, His, Ile, Lys, Leu, Met, Asn, Gln, Arg, Ser, Thr, Val, Trp or Tyr
  MISC_FEATURE (260)..(260): Xaa can be Ala, Glu, Phe, Gly, His, Ile, Leu, Met, Asn, Pro, Arg, Thr, Val, Trp or Tyr
  MISC_FEATURE (261)..(261): Xaa can be Pro, Asp, Phe, His, Ile, Leu, Met, Gln, Arg, Thr, Val, Trp or Tyr
  MISC_FEATURE (262)..(262): Xaa can be Ile, Ala, Asp, Glu, Phe, His, Lys, Leu, Met, Asn, Pro, Gln, Arg, Ser, Thr, Val, Trp or Tyr
  MISC_FEATURE (263)..(263): Xaa can be Glu, Phe, His, Ile, Leu, Met, Pro, Thr or Tyr
  MISC_FEATURE (264)..(264): Xaa can be Lys, Phe, Ile, Pro or Thr
  MISC_FEATURE (265)..(265): Xaa can be Thr, Asp, Phe, Gly, His, Ile, Leu, Met, Asn, Pro, Arg, Ser, Val, Trp or Tyr
  MISC_FEATURE (266)..(266): Xaa can be Ile, Glu, Lys or Tyr
  MISC_FEATURE (267)..(267): Xaa can be Ser, Glu, His or Asn
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPXGDXXXXCXXCPEPKSCDTPPPCPRCPEPKSCDTPPPCPRCPEPKSCDTPPPCPRCXXXXXXXXXXXXLXXXXXKXTLMISXTPXVXCXXXXXXXXXXXXXXXWXVXXXXXXXAXTXXXXXXXXXXXXXXXXLTVLHQDXLNGXXYXCXXXXXXXXXXXXXXXXKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 103 - protein, 377 aa, Artificial Sequence
  IgG3 variant including an amino acid sequence
  MISC_FEATURE (104)..(104): Xaa can be Leu or Lys
  MISC_FEATURE (112)..(112): Xaa can be Pro or Gly
  MISC_FEATURE (164)..(164): Xaa can be Leu, Tyr or Ile
  MISC_FEATURE (165)..(165): Xaa can be Leu, Tyr, Ile or Asp
  MISC_FEATURE (166)..(166): Xaa can be Gly, Ser or Ala
  MISC_FEATURE (167)..(167): Xaa can be Gly or Asp
  MISC_FEATURE (169)..(169): Xaa can be Ser, Asp, Glu, Asn, Gln or Thr
  MISC_FEATURE (170)..(170): Xaa can be Val, Ile or Met
  MISC_FEATURE (176)..(176): Xaa can be Lys, His or Tyr
  MISC_FEATURE (185)..(185): Xaa can be Arg or Tyr
  MISC_FEATURE (188)..(188): Xaa can be Glu, His or Tyr
  MISC_FEATURE (190)..(190): Xaa can be Thr or His
  MISC_FEATURE (194)..(194): Xaa can be Val, Ile, Thr or Tyr
  MISC_FEATURE (197)..(197): Xaa can be Ser, Asp or Glu
  MISC_FEATURE (198)..(198): Xaa can be Ser, Asp or Glu
  MISC_FEATURE (201)..(201): Xaa can be Pro or Gly
  MISC_FEATURE (202)..(202): Xaa can be Glu, Tyr, His, Arg or Ile
  MISC_FEATURE (204)..(204): Xaa can be Gln or Glu
  MISC_FEATURE (208)..(208): Xaa can be Tyr or Thr
  MISC_FEATURE (211)..(211): Xaa can be Gly, Asp or Glu
  MISC_FEATURE (213)..(213): Xaa can be Glu, Leu or His
  MISC_FEATURE (214)..(214): Xaa can be Val, Glu or Asp
  MISC_FEATURE (220)..(220): Xaa can be Lys or Asn
  MISC_FEATURE (223)..(223): Xaa can be Glu or Arg
  MISC_FEATURE (225)..(225): Xaa can be Gln or Glu
  MISC_FEATURE (234)..(234): Xaa can be Ser or Thr
  MISC_FEATURE (254)..(254): Xaa can be Ser, Gly or Ile
  MISC_FEATURE (256)..(256): Xaa can be Lys or Thr
  MISC_FEATURE (257)..(257): Xaa can be Ala or Asp
  MISC_FEATURE (258)..(258): Xaa can be Leu, Ala, Phe, Ile or Thr
  MISC_FEATURE (260)..(260): Xaa can be Ala, Leu, Tyr or Ile
  MISC_FEATURE (262)..(262): Xaa can be Ile, Asp, Glu, Asn, Gln or Thr
  MISC_FEATURE (263)..(263): Xaa can be Glu or Tyr
  MISC_FEATURE (264)..(264): Xaa can be Lys, Phe, Ile or Thr
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPXGDTTHTCXRCPEPKSCDTPPPCPRCPEPKSCDTPPPCPRCPEPKSCDTPPPCPRCPAPEXXXXPXXFLFPPXPKDTLMISXTPXVXCVVXDVXXEDXXVXFKWXVDXVXXHNAKTXPRXEXYNSTFRVVXVLTVLHQDWLNGKEYKCKVXNXXXPXPXXXTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 104 - protein, 330 aa, Artificial Sequence
  IgG4 variant including an amino acid sequence
  MISC_FEATURE (14)..(14): Xaa can be Cys or Ser
  MISC_FEATURE (16)..(16): Xaa can be Arg or Lys
  MISC_FEATURE (20)..(20): Xaa can be Glu or Gly
  MISC_FEATURE (21)..(21): Xaa can be Ser or Gly
  MISC_FEATURE (79)..(79): Xaa can be Lys or Gln
  MISC_FEATURE (82)..(82): Xaa can be Thr or Ile
  MISC_FEATURE (86)..(86): Xaa can be Asp or Asn
  MISC_FEATURE (97)..(97): Xaa can be Arg or Lys
  MISC_FEATURE (100)..(100): Xaa can be Ser or Pro
  MISC_FEATURE (102)..(102): Xaa can be Tyr or Ser
  MISC_FEATURE (103)..(103): Xaa can be Gly or Cys
  MISC_FEATURE (104)..(104): Xaa can be no amino acid or Asp
  MISC_FEATURE (105)..(105): Xaa can be no amino acid or Lys
  MISC_FEATURE (106)..(106): Xaa can be no amino acid or Thr
  MISC_FEATURE (107)..(107): Xaa can be Pro or His
  MISC_FEATURE (108)..(108): Xaa can be Pro or Thr
  MISC_FEATURE (111)..(111): Xaa can be Ser or Pro
  MISC_FEATURE (117)..(117): Xaa can be Phe or Leu
  MISC_FEATURE (151)..(151): Xaa can be Gln or His
  MISC_FEATURE (157)..(157): Xaa can be Gln or Lys
  MISC_FEATURE (179)..(179): Xaa can be Phe or Tyr
  MISC_FEATURE (210)..(210): Xaa can be Gly or Ala
  MISC_FEATURE (213)..(213): Xaa can be Ser or Ala
  MISC_FEATURE (214)..(214): Xaa can be Ser or Pro
  MISC_FEATURE (238)..(238): Xaa can be Gln or Arg
  MISC_FEATURE (239)..(239): Xaa can be Glu or Asp
  MISC_FEATURE (241)..(241): Xaa can be Met or Leu
  MISC_FEATURE (292)..(292): Xaa can be Arg or Lys
  MISC_FEATURE (302)..(302): Xaa can be Glu or Gln
  MISC_FEATURE (328)..(328): Xaa can be Leu or Pro
ASTKGPSVFPLAPXSXSTSXXTAALGCLVKDYFPEPVTVSWNSGALTSGVHTFPAVLQSSGLYSLSSVVTVPSSSLGTXTYXCNVXHKPSNTKVDKXVEXKXXXXXXXCPXCPAPEXLGGPSVFLFPPKPKDTLMISRTPEVTCVVVDVSXEDPEVXFNWYVDGVEVHNAKTKPREEQXNSTYRVVSVLTVLHQDWLNGKEYKCKVSNKXLPXXIEKTISKAKGQPREPQVYTLPPSXXEXTKNQVSLTCLVKGFYPSDIAVEWESNGQPENNYKTTPPVLDSDGSFFLYSXLTVDKSRWQXGNVFSCSVMHEALHNHYTQKSLSLSXGK

SEQ ID NO: 105 - protein, 330 aa, Artificial Sequence; identical to SEQ ID NO: 89
  IgG4 variant including an amino acid sequence
  MISC_FEATURE (14)..(14): Xaa can be Cys or Ser
  MISC_FEATURE (16)..(16): Xaa can be Arg or Lys
  MISC_FEATURE (20)..(20): Xaa can be Glu or Gly
  MISC_FEATURE (21)..(21): Xaa can be Ser or Gly
  MISC_FEATURE (75)..(75): Xaa can be Asn or Ser
  MISC_FEATURE (76)..(76): Xaa can be Phe or Leu
  MISC_FEATURE (79)..(79): Xaa can be Gln or Lys
  MISC_FEATURE (82)..(82): Xaa can be Gln or Lys
  MISC_FEATURE (86)..(86): Xaa can be Asp or Asn
  MISC_FEATURE (97)..(97): Xaa can be Thr, Lys or Arg
  MISC_FEATURE (100)..(100): Xaa can be Arg, Pro, Leu or Ser
  MISC_FEATURE (102)..(102): Xaa can be Cys, Ser, Thr or Tyr
  MISC_FEATURE (103)..(103): Xaa can be Cys, Pro or Gly
  MISC_FEATURE (104)..(104): Xaa can be no amino acid, Asp, Lys, Tyr, Leu, or the sequence Leu-Gly-Asp
  MISC_FEATURE (105)..(105): Xaa can be Val, Lys, Thr, no amino acid , Glu or Tyr
  MISC_FEATURE (106)..(106): Xaa can be no amino acid, Thr, Glu or Lys
  MISC_FEATURE (107)..(107): Xaa can be Glu, His, Pro or Tyr
  MISC_FEATURE (108)..(108): Xaa can be no amino acid, Thr, Pro, Glu, Lys or Trp
  MISC_FEATURE (110)..(110): Xaa can be Pro, Glu, Gly, Lys or Tyr
  MISC_FEATURE (111)..(111): Xaa can be Pro, Ser, Glu, Gly, Lys, Tyr, Arg, or SEQ ID NO:111
  MISC_FEATURE (113)..(113): Xaa can be Pro, Ala, Glu, Gly or Tyr
  MISC_FEATURE (114)..(114): Xaa can be Ala, Glu, Gly, Lys, Pro or Tyr
  MISC_FEATURE (115)..(115): Xaa can be Pro, Glu, Gly, Lys or Tyr
  MISC_FEATURE (116)..(116): Xaa can be Pro, Glu, Ala, Asp, Phe, Gly, His, Ile, Lys, Leu, Met, Asn, Gln, Arg, Ser, Thr, Val, Trp or Tyr
  MISC_FEATURE (117)..(117): Xaa can be Val, Leu, Phe, Asp, Glu, Phe, Gly, His, Ile, Lys, Met, Asn, Pro, Gln, Arg, Ser, Thr, Trp or Tyr
  MISC_FEATURE (118)..(118): Xaa can be Ala, Leu, Asp, Phe, Gly, His, Ile, Lys, Met, Asn, Pro, Gln, Arg, Ser, Thr, Val, Trp or Tyr
  MISC_FEATURE (119)..(119): Xaa can be no amino acid, Gly, Ala, Asp, Glu, Phe, His, Ile, Lys, Leu, Met, Asn, Pro, Gln, Arg, Ser, Thr, Val, Trp or Tyr
  MISC_FEATURE (120)..(120): Xaa can be Gly, Asp, Glu, Phe, His, Ile, Lys, Leu, Met, Asn, Pro, Gln, Arg, Ser, Thr, Val, Trp or Tyr;
  MISC_FEATURE (121)..(121): Xaa can be Pro, Asp, Glu, Phe, Gly, His, Ile, Lys, Leu, Met, Asn, Gln, Arg, Ser, Thr, Val, Trp or Tyr
  MISC_FEATURE (122)..(122): Xaa can be Ser, Asp, Glu, Phe, Gly, His, Ile, Lys, Leu, Met, Asn, Pro, Gln, Arg, Thr, Val, Trp or Tyr
  MISC_FEATURE (123)..(123): Xaa can be Val, Ala, Ile, Met or Thr
  MISC_FEATURE (124)..(124): Xaa can be Phe, Asp, Glu, Leu, Arg, Ser, Trp or Tyr
  MISC_FEATURE (126)..(126): Xaa can be Phe, Glu, His, Leu, Gln, Arg, Trp, or Tyr
  MISC_FEATURE (127)..(127): Xaa can be Pro or His
  MISC_FEATURE (128)..(128): Xaa can be Pro or Ala
  MISC_FEATURE (129)..(129): Xaa can be , Lys, Asp, Glu, His or Tyr
  MISC_FEATURE (130)..(130): Xaa can be Pro, Gly or Val
  MISC_FEATURE (132)..(132): Xaa can be Asp, His, Gln or Tyr
  MISC_FEATURE (138)..(138): Xaa can be Arg, Glu or Tyr
  MISC_FEATURE (141)..(141): Xaa can be Glu, His, Ser or Tyr
  MISC_FEATURE (143)..(143): Xaa can be Thr, Asp, Glu, His or Tyr
  MISC_FEATURE (145)..(145): Xaa can be Val, Ala, Glu, Phe, Ile or Thr
  MISC_FEATURE (146)..(146): Xaa can be Val, Ala, Ile, Met or Thr
  MISC_FEATURE (147)..(147): Xaa can be Val, Ala, Asp, Glu, Phe, Gly, His, Ile, Lys, Leu, Met, Asn, Pro, Gln, Arg, Ser, Thr, Trp, or Tyr
  MISC_FEATURE (148)..(148): Xaa can be Asp, Phe, Gly, His, Ile, Lys, Leu, Met, Pro, Gln, Arg, Ser, Thr, Val, Trp or Tyr
  MISC_FEATURE (149)..(149): Xaa can be Val, Ala, Ile, Met or Thr
  MISC_FEATURE (150)..(150): Xaa can be Ser, Asp, Glu, Phe, His, Ile, Lys, Leu, Met, Asn, Pro, Gln, Arg, Val, Trp or Tyr
  MISC_FEATURE (151)..(151): Xaa can be His, Gln, Asp, Glu, Phe, Gly, Ile, Lys, Leu, Met, Pro, Arg, Thr, Val or Trp
  MISC_FEATURE (152)..(152): Xaa can be Glu, Phe, Gly, His, Ile, Lys, Leu, Met, Asn, Pro, Arg, Ser, Thr, Val, Trp or Tyr
  MISC_FEATURE (153)..(153): Xaa can be Asp, Phe, Gly, His, Ile, Leu, Met, Pro, Gln, Arg, Ser, Thr, Trp or Tyr
  MISC_FEATURE (154)..(154): Xaa can be Pro, Ala, Asp, Glu, Phe, Gly, His, Ile, Lys, Leu, Met, Asn, Gln, Arg, Ser, Thr, Val, Trp or Tyr
  MISC_FEATURE (155)..(155): Xaa can be Glu, Asp, Phe, Gly, His, Ile, Lys, Leu, Met, Pro, Arg, Ser, Thr, Val, Trp or Tyr
  MISC_FEATURE (156)..(156): Xaa can be Val or Ile
  MISC_FEATURE (157)..(157): Xaa can be Gln, Lys, Asp, Glu, Phe, Gly, His, Ile, Leu, Met, Asn, Pro, Arg, Thr, Val, Trp or Tyr
  MISC_FEATURE (158)..(158): Xaa can be Phe, Leu or Trp
  MISC_FEATURE (159)..(159): Xaa can be Asn, Lys, Asp, Glu, Phe, Gly, His, Ile, Leu, Met, Pro, Arg, Ser, Thr, Val, Trp or Tyr
  MISC_FEATURE (161)..(161): Xaa can be Tyr, Asp, Glu, Gly, His, Ile, Lys, Leu, Met, Asn, Pro, Gln, Arg, Ser, Thr, Val or Trp
  MISC_FEATURE (163)..(163): Xaa can be Asp, Gly, Lys, Leu, Pro or Trp
  MISC_FEATURE (164)..(164): Xaa can be Gly, Asp, Glu, Lys, Asn, Pro, Gln or Tyr
  MISC_FEATURE (165)..(165): Xaa can be Val, Glu, Gly, Lys, Pro or Tyr
  MISC_FEATURE (166)..(166): Xaa can be Glu, Gly, His, Lys, Leu, Pro, Arg or Tyr
  MISC_FEATURE (167)..(167): Xaa can be Val, Asp, Glu, Leu, Asn, Gln, Thr or Tyr
  MISC_FEATURE (168)..(168): Xaa can be His, Asp, Glu, Lys, Gln, Trp or Tyr
  MISC_FEATURE (169)..(169): Xaa can be Asn, Glu, Gly, Pro or Tyr
  MISC_FEATURE (171)..(171): Xaa can be Asn, Glu, Gly, Pro or Tyr
  MISC_FEATURE (173)..(173): Xaa can be Lys, Asp, His, Leu, Asn or Trp
  MISC_FEATURE (174)..(174): Xaa can be Pro, Asp, Glu, Gly, His, Ile, Gln or Thr
  MISC_FEATURE (175)..(175): Xaa can be Arg, Asp, Glu, Thr or Tyr
  MISC_FEATURE (176)..(176): Xaa can be Glu, Phe, Gly, His, Ile, Leu, Met, Asn, Pro, Arg, Ser, Thr, Val, Trp or Tyr
  MISC_FEATURE (177)..(177): Xaa can be Glu, Phe, Gly, His, Ile, Lys, Leu, Met, Pro, Arg, Ser, Thr, Val, Trp or Tyr
  MISC_FEATURE (178)..(178): Xaa can be Gln, Asp, Glu, Phe, Gly, His, Ile, Met, Asn, Pro, Arg, Ser, Thr, Val, Trp or Tyr
  MISC_FEATURE (179)..(179): Xaa can be Phe, Tyr, Ala, Asp, Glu, Gly, Ile, Lys, Leu, Met, Asn, Gln, Arg, Ser, Thr or Val
  MISC_FEATURE (180)..(180): Xaa can be Asn, Asp, Glu, Phe, Gly, His, Ile, Lys, Leu, Met, Pro, Gln, Arg, Ser, Thr, Val, Trp or Tyr
  MISC_FEATURE (181)..(181): Xaa can be Ser, Glu, Phe, His, Ile, Lys, Met, Gln, Arg, Trp or Tyr
  MISC_FEATURE (182)..(182): Xaa can be Thr, Ala, Asp, Glu, Phe, Gly, His, Ile, Lys, Leu, Met, Asn, Pro, Gln, Arg, Ser, Val, Trp or Tyr
  MISC_FEATURE (183)..(183): Xaa can be Phe, Tyr, Ala, Asp, Glu, Gly, His, Lys, Met, Asn, Pro, Gln, Arg, Ser, Thr, Val or Trp
  MISC_FEATURE (184)..(184): Xaa can be Arg, Asp, Glu, His or Tyr
  MISC_FEATURE (185)..(185): Xaa can be Val or Ile
  MISC_FEATURE (186)..(186): Xaa can be Val, Asp, Glu or Tyr
  MISC_FEATURE (187)..(187): Xaa can be Ser, Asp, His, Leu, Asn or Thr
  MISC_FEATURE (188)..(188): Xaa can be Val, Glu, Thr or Tyr
  MISC_FEATURE (192)..(192): Xaa can be Val or Leu
  MISC_FEATURE (196)..(196): Xaa can be Trp or Phe
  MISC_FEATURE (200)..(200): Xaa can be Lys, Glu or Gln
  MISC_FEATURE (201)..(201): Xaa can be Glu, His, Leu, Gln, Arg or Tyr
  MISC_FEATURE (203)..(203): Xaa can be Lys, Asp, Phe, Gly, His, Ile, Leu, Asn, Pro, Ser, Thr, Val, Trp or Tyr
  MISC_FEATURE (205)..(205): Xaa can be Lys, Asp, Phe, Gly, His, Ile, Pro, Ser, Thr, Val, Trp or Tyr
  MISC_FEATURE (206)..(206): Xaa can be Val or Ile
  MISC_FEATURE (207)..(207): Xaa can be Ser, Asp, Phe, Gly, His, Ile, Leu, Met, Pro, Arg, Thr, Val, Trp or Tyr
  MISC_FEATURE (208)..(208): Xaa can be Asn, Ala, Asp, Glu, Phe, Gly, His, Ile, Lys, Leu, Met, Pro, Gln, Arg, Ser, Thr, Val, Trp or Tyr
  MISC_FEATURE (209)..(209): Xaa can be Lys, Ile, Leu, Pro or Thr
  MISC_FEATURE (210)..(210): Xaa can be Gly, Ala, Asp, Glu, Phe, His, Ile, Lys, Leu, Met, Asn, Pro, Arg, Thr, Val, Trp or Tyr
  MISC_FEATURE (211)..(211): Xaa can be Leu, Ala, Asp, Glu, Phe, Gly, His, Ile, Lys, Met, Asn, Pro, Gln, Arg, Ser, Thr, Val, Trp or Tyr
  MISC_FEATURE (212)..(212): Xaa can be Pro, Asp, Glu, Phe, Gly, His, Ile, Lys, Leu, Met, Asn, Gln, Arg, Ser, Thr, Val, Trp or Tyr
  MISC_FEATURE (213)..(213): Xaa can be Ala, Ser, Glu, Phe, Gly, His, Ile, Leu, Met, Asn, Pro, Arg, Thr, Val, Trp or Tyr
  MISC_FEATURE (214)..(214): Xaa can be Ala, Ser, Glu, Phe, Gly, His, Ile, Leu, Met, Asn, Pro, Arg, Thr, Val, Trp or Tyr
  MISC_FEATURE (215)..(215): Xaa can be Ile, Ala, Asp, Glu, Phe, His, , Lys, Leu, Met, Asn, Pro, Gln, Arg, Ser, Thr, Val, Trp or Tyr
  MISC_FEATURE (216)..(216): Xaa can be Glu, Phe, His, Ile, Leu, Met, Pro, Thr or Tyr
  MISC_FEATURE (217)..(217): Xaa can be Lys, Phe, Ile, Pro or Thr
  MISC_FEATURE (218)..(218): Xaa can be Thr, Asp, Phe, Gly, His, Ile, Leu, Met, Asn, Pro, Arg, Ser, Val, Trp or Tyr
  MISC_FEATURE (219)..(219): Xaa can be Ile, Glu, Lys or Tyr
  MISC_FEATURE (220)..(220): Xaa can be Ser, Glu, His or Asn
  MISC_FEATURE (222)..(222): Xaa can be Thr or Ala
  MISC_FEATURE (238)..(238): Xaa can be Arg or Gln
  MISC_FEATURE (239)..(239): Xaa can be Glu or Asp
  MISC_FEATURE (241)..(241): Xaa can be Met or Leu
  MISC_FEATURE (267)..(267): Xaa can be Asn or Ser
  MISC_FEATURE (275)..(275): Xaa can be Lys or Asn
  MISC_FEATURE (280)..(280): Xaa can be Met or Val
  MISC_FEATURE (292)..(292): Xaa can be Lys or Arg
  MISC_FEATURE (302)..(302): Xaa can be Gln or Glu
  MISC_FEATURE (305)..(305): Xaa can be Val or Ile
  MISC_FEATURE (318)..(318): Xaa can be His or Arg
  MISC_FEATURE (319)..(319): Xaa can be Tyr or Phe
  MISC_FEATURE (328)..(328): Xaa can be Pro or Leu

SEQ ID NO: 106 - protein, 330 aa, Artificial Sequence; identical to SEQ ID NO: 90
  IgG4 variant including an amino acid sequence
  MISC_FEATURE (14)..(14): Xaa can be Cys or Ser
  MISC_FEATURE (16)..(16): Xaa can be Arg or Lys
  MISC_FEATURE (20)..(20): Xaa can be Glu or Gly
  MISC_FEATURE (21)..(21): Xaa can be Ser or Gly
  MISC_FEATURE (75)..(75): Xaa can be Asn or Ser
  MISC_FEATURE (76)..(76): Xaa can be Phe or Leu
  MISC_FEATURE (79)..(79): Xaa can be Gln or Lys
  MISC_FEATURE (82)..(82): Xaa can be Thr or Ile
  MISC_FEATURE (86)..(86): Xaa can be Asp or Asn
  MISC_FEATURE (97)..(97): Xaa can be Thr, Lys or Arg
  MISC_FEATURE (100)..(100): Xaa can be Arg, Pro, Leu or Ser
  MISC_FEATURE (102)..(102): Xaa can be Cys, Ser, Thr or Tyr
  MISC_FEATURE (103)..(103): Xaa can be Cys, Pro or Gly
  MISC_FEATURE (104)..(104): Xaa can be no amino acid, Asp, Leu, Lys, or the sequence Leu-Gly-Asp
  MISC_FEATURE (105)..(105): Xaa can be Val, Lys, Thr, or no amino acid
  MISC_FEATURE (106)..(106): Xaa can be no amino acid or Thr
  MISC_FEATURE (107)..(107): Xaa can be Glu, His or Pro
  MISC_FEATURE (108)..(108): Xaa can be no amino acid, Thr or Pro
  MISC_FEATURE (110)..(110): Xaa can be Pro or Gly
  MISC_FEATURE (111)..(111): Xaa can be Pro, Arg, Ser, or SEQ ID NO:111
  MISC_FEATURE (116)..(116): Xaa can be Pro or Glu
  MISC_FEATURE (117)..(117): Xaa can be Val, Leu, Phe, Tyr or Ile
  MISC_FEATURE (118)..(118): Xaa can be Ala, Leu, Tyr, Ile or Asp
  MISC_FEATURE (119)..(119): Xaa can be Ala, Leu, Tyr, Ile or Asp
  MISC_FEATURE (120)..(120): Xaa can be Gly or Asp
  MISC_FEATURE (122)..(122): Xaa can be Ser, Asp, Glu, Asn, Gln or Thr
  MISC_FEATURE (123)..(123): Xaa can be Val, Ile or Met
  MISC_FEATURE (129)..(129): Xaa can be Lys, His or Tyr
  MISC_FEATURE (138)..(138): Xaa can be Arg or Tyr
  MISC_FEATURE (141)..(141): Xaa can be Glu, His or Tyr
  MISC_FEATURE (143)..(143): Xaa can be Thr or His
  MISC_FEATURE (147)..(147): Xaa can be Val, Ile, Thr or Tyr
  MISC_FEATURE (150)..(150): Xaa can be Ser, Asp or Glu
  MISC_FEATURE (151)..(151): Xaa can be His, Gln, Asp or Glu
  MISC_FEATURE (154)..(154): Xaa can be Pro or Gly
  MISC_FEATURE (155)..(155): Xaa can be Pro or Gly
  MISC_FEATURE (157)..(157): Xaa can be Gln, Lys or Glu
  MISC_FEATURE (159)..(159): Xaa can be Asn or Lys
  MISC_FEATURE (161)..(161): Xaa can be Tyr or Thr
  MISC_FEATURE (164)..(164): Xaa can be Gly, Asp or Glu
  MISC_FEATURE (166)..(166): Xaa can be Glu, Leu or His
  MISC_FEATURE (167)..(167): Xaa can be Val, Glu or Asp
  MISC_FEATURE (173)..(173): Xaa can be Lys or Asn
  MISC_FEATURE (176)..(176): Xaa can be Glu or Arg
  MISC_FEATURE (178)..(178): Xaa can be Gln or Glu
  MISC_FEATURE (179)..(179): Xaa can be Phe or Tyr
  MISC_FEATURE (183)..(183): Xaa can be Phe or Tyr
  MISC_FEATURE (187)..(187): Xaa can be Ser or Thr
  MISC_FEATURE (192)..(192): Xaa can be Val or Leu
  MISC_FEATURE (207)..(207): Xaa can be Ser, Gly or Ile
  MISC_FEATURE (209)..(209): Xaa can be Ser, Gly or Ile
  MISC_FEATURE (210)..(210): Xaa can be Gly, Ala or Asp
  MISC_FEATURE (211)..(211): Xaa can be Leu, Ala, Phe, Ile or Thr
  MISC_FEATURE (213)..(213): Xaa can be Ala, Ser, Leu, Tyr or Ile
  MISC_FEATURE (214)..(214): Xaa can be Pro or Ser
  MISC_FEATURE (215)..(215): Xaa can be Ile, Asp, Glu, Asn, Gln or Thr
  MISC_FEATURE (216)..(216): Xaa can be Glu or Tyr
  MISC_FEATURE (217)..(217): Xaa can be Lys, Phe, Ile or Thr
  MISC_FEATURE (222)..(222): Xaa can be Thr or Ala
  MISC_FEATURE (238)..(238): Xaa can be Arg or Gln
  MISC_FEATURE (239)..(239): Xaa can be Glu or Asp
  MISC_FEATURE (241)..(241): Xaa can be Met or Leu
  MISC_FEATURE (267)..(267): Xaa can be Asn or Ser
  MISC_FEATURE (275)..(275): Xaa can be Lys or Asn
  MISC_FEATURE (280)..(280): Xaa can be Met or Val
  MISC_FEATURE (292)..(292): Xaa can be Lys or Arg
  MISC_FEATURE (302)..(302): Xaa can be Gln or Glu
  MISC_FEATURE (305)..(305): Xaa can be Val or Ile
  MISC_FEATURE (318)..(318): Xaa can be His or Arg
  MISC_FEATURE (319)..(319): Xaa can be Tyr or Phe
  MISC_FEATURE (328)..(328): Xaa can be Pro or Leu

SEQ ID NO: 107 - protein, 222 aa, Artificial Sequence; identical to SEQ ID NO: 100
  IgG4 variant including an amino acid sequence
  MISC_FEATURE (2)..(2): Xaa can be Pro, Glu, Gly, Lys or Tyr
  MISC_FEATURE (3)..(3): Xaa can be Pro, Ser, Glu, Gly, Lys, Tyr, Arg, or SEQ ID NO:111
  MISC_FEATURE (5)..(5): Xaa can be Pro, Ala, Glu, Gly or Tyr
  MISC_FEATURE (6)..(6): Xaa can be Ala, Glu, Gly, Lys, Pro or Tyr
  MISC_FEATURE (7)..(7): Xaa can be Pro, Glu, Gly, Lys or Tyr
  MISC_FEATURE (8)..(8): Xaa can be Pro, Glu, Ala, Asp, Phe, Gly, His, Ile, Lys, Leu, Met, Asn, Gln, Arg, Ser, Thr, Val, Trp or Tyr
  MISC_FEATURE (9)..(9): Xaa can be Val, Leu, Phe, Asp, Glu, Phe, Gly, His, Ile, Lys, Met, Asn, Pro, Gln, Arg, Ser, Thr, Trp or Tyr
  MISC_FEATURE (10)..(10): Xaa can be Ala, Leu, Asp, Phe, Gly, His, Ile, Lys, Met, Asn, Pro, Gln, Arg, Ser, Thr, Val, Trp , or Tyr
  MISC_FEATURE (11)..(11): Xaa can be no amino acid, Gly, Ala, Asp, Glu, Phe, His, Ile, Lys, Leu, Met, Asn, Pro, Gln, Arg, Ser, Thr, Val, Trp or Tyr
  MISC_FEATURE (12)..(12): Xaa can be Gly, Asp, Glu, Phe, His, Ile, Lys, Leu, Met, Asn, Pro, Gln, Arg, Ser, Thr, Val, Trp or Tyr
  MISC_FEATURE (13)..(13): Xaa can be Pro, Asp, Glu, Phe, Gly, His, Ile, Lys, Leu, Met, Asn, Gln, Arg, Ser, Thr, Val, Trp or Tyr
  MISC_FEATURE (14)..(14): Xaa can be Ser, Asp, Glu, Phe, Gly, His, Ile, Lys, Leu, Met, Asn, Pro, Gln, Arg, Thr, Val, Trp or Tyr
  MISC_FEATURE (15)..(15): Xaa can be Val, Ala, Ile, Met or Thr
  MISC_FEATURE (16)..(16): Xaa can be Val, Ala, Ile, Met or Thr
  MISC_FEATURE (18)..(18): Xaa can be Val, Ala, Ile, Met or Thr
  MISC_FEATURE (19)..(19): Xaa can be Pro or His
  MISC_FEATURE (20)..(20): Xaa can be Pro or Ala
  MISC_FEATURE (21)..(21): Xaa can be , Lys, Asp, Glu, His or Tyr
  MISC_FEATURE (22)..(22): Xaa can be Pro, Gly or Val
  MISC_FEATURE (24)..(24): Xaa can be Asp, His, Gln or Tyr
  MISC_FEATURE (30)..(30): Xaa can be Arg, Glu or Tyr
  MISC_FEATURE (33)..(33): Xaa can be Glu, His, Ser or Tyr;
  MISC_FEATURE (35)..(35): Xaa can be Thr, Asp, Glu, His or Tyr
  MISC_FEATURE (37)..(37): Xaa can be Val, Ala, Glu, Phe, Ile or Thr
  MISC_FEATURE (38)..(38): Xaa can be Val, Ala, Ile, Met or Thr
  MISC_FEATURE (39)..(39): Xaa can be Val, Ala, Asp, Glu, Phe, Gly, His, Ile, Lys, Leu, Met, Asn, Pro, Gln, Arg, Ser, Thr, Trp, or Tyr
  MISC_FEATURE (40)..(40): Xaa can be Asp, Phe, Gly, His, Ile, Lys, Leu, Met, Pro, Gln, Arg, Ser, Thr, Val, Trp or Tyr
  MISC_FEATURE (41)..(41): Xaa can be Val, Ala, Ile, Met or Thr
  MISC_FEATURE (42)..(42): Xaa can be Ser, Asp, Glu, Phe, His, Ile, Lys, Leu, Met, Asn, Pro, Gln, Arg, Val, Trp or Tyr
  MISC_FEATURE (43)..(43): Xaa can be His, Gln, Asp, Glu, Phe, Gly, Ile, Lys, Leu, Met, Pro, Arg, Thr, Val or Trp
  MISC_FEATURE (44)..(44): Xaa can be Glu, Phe, Gly, His, Ile, Lys, Leu, Met, Asn, Pro, Arg, Ser, Thr, Val, Trp or Tyr
  MISC_FEATURE (45)..(45): Xaa can be Asp, Phe, Gly, His, Ile, Leu, Met, Pro, Gln, Arg, Ser, Thr, Trp or Tyr
  MISC_FEATURE (46)..(46): Xaa can be Pro, Ala, Asp, Glu, Phe, Gly, His, Ile, Lys, Leu, Met, Asn, Gln, Arg, Ser, Thr, Val, Trp or Tyr
  MISC_FEATURE (47)..(47): Xaa can be Glu, Asp, Phe, Gly, His, Ile, Lys, Leu, Met, Pro, Arg, Ser, Thr, Val, Trp or Tyr
  MISC_FEATURE (48)..(48): Xaa can be Val or Ile
  MISC_FEATURE (49)..(49): Xaa can be Gln, Lys, Asp, Glu, Phe, Gly, His, Ile, Leu, Met, Asn, Pro, Arg, Thr, Val, Trp or Tyr
  MISC_FEATURE (50)..(50): Xaa can be Phe, Leu or Trp
  MISC_FEATURE (51)..(51): Xaa can be Asn, Lys, Asp, Glu, Phe, Gly, His, Ile, Leu, Met, Pro, Arg, Ser, Thr, Val, Trp or Tyr
  MISC_FEATURE (53)..(53): Xaa can be Tyr, Asp, Glu, Gly, His, Ile, Lys, Leu, Met, Asn, Pro, Gln, Arg, Ser, Thr, Val or Trp
  MISC_FEATURE (55)..(55): Xaa can be Asp, Gly, Lys, Leu, Pro or Trp
  MISC_FEATURE (56)..(56): Xaa can be Gly, Asp, Glu, Lys, Asn, Pro, Gln or Tyr
  MISC_FEATURE (57)..(57): Xaa can be Val, Glu, Gly, Lys, Pro or Tyr
  MISC_FEATURE (58)..(58): Xaa can be Val, Glu, Gly, Lys, Pro or Tyr
  MISC_FEATURE (59)..(59): Xaa can be Val, Asp, Glu, Leu, Asn, Gln, Thr or Tyr
  MISC_FEATURE (60)..(60): Xaa can be His, Asp, Glu, Lys, Gln, Trp or Tyr
  MISC_FEATURE (61)..(61): Xaa can be Asn, Glu, Gly, Pro or Tyr
  MISC_FEATURE (63)..(63): Xaa can be Lys, Asp, Glu or Tyr
  MISC_FEATURE (65)..(65): Xaa can be Lys, Asp, His, Leu, Asn or Trp
  MISC_FEATURE (66)..(66): Xaa can be Pro, Asp, Glu, Gly, His, Ile, Gln or Thr
  MISC_FEATURE (67)..(67): Xaa can be Arg, Asp, Glu, Thr or Tyr
  MISC_FEATURE (68)..(68): Xaa can be Glu, Phe, Gly, His, Ile, Leu, Met, Asn, Pro, Arg, Ser, Thr, Val, Trp or Tyr
  MISC_FEATURE (69)..(69): Xaa can be Glu, Phe, Gly, His, Ile, Lys, Leu, Met, Pro, Arg, Ser, Thr, Val, Trp or Tyr
  MISC_FEATURE (70)..(70): Xaa can be Gln, Asp, Glu, Phe, Gly, His, Ile, Met, Asn, Pro, Arg, Ser, Thr, Val, Trp or Tyr
  MISC_FEATURE (71)..(71): Xaa can be Phe, Tyr, Ala, Asp, Glu, Gly, Ile, Lys, Leu, Met, Asn, Gln, Arg, Ser, Thr or Val
  MISC_FEATURE (72)..(72): Xaa can be Asn, Asp, Glu, Phe, Gly, His, Ile, Lys, Leu, Met, Pro, Gln, Arg, Ser, Thr, Val, Trp or Tyr
  MISC_FEATURE (73)..(73): Xaa can be Ser, Glu, Phe, His, Ile, Lys, Met, Gln, Arg, Trp or Tyr
  MISC_FEATURE (74)..(74): Xaa can be Thr, Ala, Asp, Glu, Phe, Gly, His, Ile, Lys, Leu, Met, Asn, Pro, Gln, Arg, Ser, Val, Trp or Tyr
  MISC_FEATURE (75)..(75): Xaa can be Phe, Tyr, Ala, Asp, Glu, Gly, His, Lys, Met, Asn, Pro, Gln, Arg, Ser, Thr, Val or Trp
  MISC_FEATURE (76)..(76): Xaa can be Arg, Asp, Glu, His or Tyr
  MISC_FEATURE (77)..(77): Xaa can be Val or Ile
  MISC_FEATURE (78)..(78): Xaa can be Val, Asp, Glu or Tyr
  MISC_FEATURE (79)..(79): Xaa can be Ser, Asp, His, Leu, Asn or Thr
  MISC_FEATURE (80)..(80): Xaa can be Val, Glu, Thr or Tyr
  MISC_FEATURE (84)..(84): Xaa can be Val or Leu
  MISC_FEATURE (88)..(88): Xaa can be Trp or Phe
  MISC_FEATURE (92)..(92): Xaa can be Lys, Glu or Gln
  MISC_FEATURE (93)..(93): Xaa can be Glu, His, Leu, Gln, Arg or Tyr
  MISC_FEATURE (95)..(95): Xaa can be Lys, Asp, Phe, Gly, His, Ile, Leu, Asn, Pro, Ser, Thr, Val, Trp or Tyr
  MISC_FEATURE (97)..(97): Xaa can be Lys, Asp, Phe, Gly, His, Ile, Pro, Ser, Thr, Val, Trp or Tyr
  MISC_FEATURE (98)..(98): Xaa can be Val or Ile
  MISC_FEATURE (99)..(99): Xaa can be Ser, Asp, Phe, Gly, His, Ile, Leu, Met, Pro, Arg, Thr, Val, Trp or Tyr
  MISC_FEATURE (100)..(100): Xaa can be Asn, Ala, Asp, Glu, Phe, Gly, His, Ile, Lys, Leu, Met, Pro, Gln, Arg, Ser, Thr, Val, Trp or Tyr
  MISC_FEATURE (101)..(101): Xaa can be Lys, Ile, Leu, Pro or Thr
  MISC_FEATURE (102)..(102): Xaa can be Gly, Ala, Asp, Glu, Phe, His, Ile, Lys, Leu, Met, Asn, Pro, Arg, Thr, Val, Trp or Tyr
  MISC_FEATURE (103)..(103): Xaa can be Leu, Ala, Asp, Glu, Phe, Gly, His, Ile, Lys, Met, Asn, Pro, Gln, Arg, Ser, Thr, Val, Trp or Tyr
  MISC_FEATURE (104)..(104): Xaa can be Pro, Asp, Glu, Phe, Gly, His, Ile, Lys, Leu, Met, Asn, Gln, Arg, Ser, Thr, Val, Trp or Tyr
  MISC_FEATURE (105)..(105): Xaa can be Ala, Ser, Glu, Phe, Gly, His, Ile, Leu, Met, Asn, Pro, Arg, Thr, Val, Trp or Tyr
  MISC_FEATURE (106)..(106): Xaa can be Pro, Ser, Asp, Phe, His, Ile, Leu, Met, Gln, Arg, Thr, Val, Trp or Tyr
  MISC_FEATURE (107)..(107): Xaa can be Ile, Ala, Asp, Glu, Phe, His, , Lys, Leu, Met, Asn, Pro, Gln, Arg, Ser, Thr, Val, Trp or Tyr
  MISC_FEATURE (108)..(108): Xaa can be Glu, Phe, His, Ile, Leu, Met, Pro, Thr or Tyr
  MISC_FEATURE (109)..(109): Xaa can be Lys, Phe, Ile, Pro or Thr
  MISC_FEATURE (110)..(110): Xaa can be Thr, Asp, Phe, Gly, His, Ile, Leu, Met, Asn, Pro, Arg, Ser, Val, Trp or Tyr
  MISC_FEATURE (111)..(111): Xaa can be Ile, Glu, Lys or Tyr
  MISC_FEATURE (112)..(112): Xaa can be Ser, Glu, His or Asn
  MISC_FEATURE (114)..(114): Xaa can be Thr or Ala
  MISC_FEATURE (130)..(130): Xaa can be Arg or Gln
  MISC_FEATURE (131)..(131): Xaa can be Glu or Asp
  MISC_FEATURE (133)..(133): Xaa can be Met or Leu
  MISC_FEATURE (159)..(159): Xaa can be Asn or Ser
  MISC_FEATURE (167)..(167): Xaa can be Lys or Asn
  MISC_FEATURE (172)..(172): Xaa can be Met or Val
  MISC_FEATURE (184)..(184): Xaa can be Lys or Arg
  MISC_FEATURE (194)..(194): Xaa can be Gln or Glu
  MISC_FEATURE (197)..(197): Xaa can be Val or Ile
  MISC_FEATURE (210)..(210): Xaa can be His or Arg
  MISC_FEATURE (211)..(211): Xaa can be Tyr or Phe
  MISC_FEATURE (220)..(220): Xaa can be Pro or Leu

SEQ ID NO: 108 - protein, 222 aa, Artificial Sequence; identical to SEQ ID NO: 101
  IgG4 variant including an amino acid sequence
  MISC_FEATURE (2)..(2): Xaa can be Pro or Gly
  MISC_FEATURE (3)..(3): Xaa can be Pro, Arg, Ser, or SEQ ID NO:111
  MISC_FEATURE (8)..(8): Xaa can be Pro or Glu
  MISC_FEATURE (9)..(9): Xaa can be Val, Leu, Phe, Tyr or Ile
  MISC_FEATURE (10)..(10): Xaa can be Ala, Leu, Tyr, Ile or Asp
  MISC_FEATURE (11)..(11): Xaa can be no amino acid, Gly, Ser or Ala
  MISC_FEATURE (12)..(12): Xaa can be Gly or Asp
  MISC_FEATURE (14)..(14): Xaa can be Ser, Asp, Glu, Asn, Gln or Thr
  MISC_FEATURE (15)..(15): Xaa can be Val, Ile or Met
  MISC_FEATURE (21)..(21): Xaa can be Lys, His or Tyr
  MISC_FEATURE (30)..(30): Xaa can be Arg or Tyr
  MISC_FEATURE (33)..(33): Xaa can be Glu, His or Tyr
  MISC_FEATURE (35)..(35): Xaa can be Thr or His
  MISC_FEATURE (39)..(39): Xaa can be Val, Ile, Thr or Tyr
  MISC_FEATURE (42)..(42): Xaa can be Ser, Asp or Glu
  MISC_FEATURE (43)..(43): Xaa can be His, Gln, Asp or Glu
  MISC_FEATURE (46)..(46): Xaa can be Pro or Gly
  MISC_FEATURE (47)..(47): Xaa can be Glu, Tyr, His, Arg or Ile
  MISC_FEATURE (49)..(49): Xaa can be Gln, Lys or Glu
  MISC_FEATURE (51)..(51): Xaa can be Asn or Lys
  MISC_FEATURE (53)..(53): Xaa can be Tyr or Thr
  MISC_FEATURE (56)..(56): Xaa can be Gly, Asp or Glu
  MISC_FEATURE (58)..(58): Xaa can be Glu, Leu or His
  MISC_FEATURE (59)..(59): Xaa can be Val, Glu or Asp
  MISC_FEATURE (65)..(65): Xaa can be Lys or Asn
  MISC_FEATURE (68)..(68): Xaa can be Glu or Arg
  MISC_FEATURE (70)..(70): Xaa can be Gln or Glu
  MISC_FEATURE (71)..(71): Xaa can be Phe or Tyr
  MISC_FEATURE (75)..(75): Xaa can be Phe or Tyr
  MISC_FEATURE (79)..(79): Xaa can be Ser or Thr
  MISC_FEATURE (84)..(84): Xaa can be Val or Leu
  MISC_FEATURE (99)..(99): Xaa can be Ser, Gly or Ile
  MISC_FEATURE (101)..(101): Xaa can be Lys or Thr
  MISC_FEATURE (102)..(102): Xaa can be Gly, Ala or Asp
  MISC_FEATURE (103)..(103): Xaa can be Leu, Ala, Phe, Ile or Thr
  MISC_FEATURE (105)..(105): Xaa can be Ala, Ser, Leu, Tyr or Ile
  MISC_FEATURE (106)..(106): Xaa can be Pro or Ser
  MISC_FEATURE (107)..(107): Xaa can be Ile, Asp, Glu, Asn, Gln or Thr
  MISC_FEATURE (108)..(108): Xaa can be Glu or Tyr
  MISC_FEATURE (109)..(109): Xaa can be Lys, Phe, Ile or Thr
  MISC_FEATURE (114)..(114): Xaa can be Thr or Ala
  MISC_FEATURE (130)..(130): Xaa can be Arg or Gln
  MISC_FEATURE (131)..(131): Xaa can be Glu or Asp
  MISC_FEATURE (133)..(133): Xaa can be Met or Leu
  MISC_FEATURE (159)..(159): Xaa can be Met or Leu
  MISC_FEATURE (167)..(167): Xaa can be Lys or Asn
  MISC_FEATURE (172)..(172): Xaa can be Met or Val
  MISC_FEATURE (184)..(184): Xaa can be Lys or Arg
  MISC_FEATURE (194)..(194): Xaa can be Gln or Glu
  MISC_FEATURE (197)..(197): Xaa can be Val or Ile
  MISC_FEATURE (210)..(210): Xaa can be His or Arg
  MISC_FEATURE (211)..(211): Xaa can be Tyr or Phe
  MISC_FEATURE (220)..(220): Xaa can be Pro or Leu

SEQ ID NO: 109 - protein, 330 aa, Artificial Sequence
  IgG4 variant including an amino acid sequence
  MISC_FEATURE (104)..(104): Xaa can be no amino acid, Lys or Tyr
  MISC_FEATURE (105)..(105): Xaa can be no amino acid, Glu or Tyr
  MISC_FEATURE (106)..(106): Xaa can be no amino acid , Glu or Lys
  MISC_FEATURE (107)..(107): Xaa can be Pro or Tyr
  MISC_FEATURE (108)..(108): Xaa can be Pro, Glu, Lys or Trp
  MISC_FEATURE (110)..(110): Xaa can be Pro, Glu, Gly, Lys or Tyr
  MISC_FEATURE (111)..(111): Xaa can be Ser, Glu, Gly, Lys or Tyr
  MISC_FEATURE (113)..(113): Xaa can be Pro, Ala, Glu, Gly or Tyr
  MISC_FEATURE (114)..(114): Xaa can be Ala, Glu, Gly, Lys, Pro or Tyr
  MISC_FEATURE (115)..(115): Xaa can be Pro, Glu, Gly, Lys or Tyr
  MISC_FEATURE (116)..(116): Xaa can be Glu, Ala, Asp, Phe, Gly, His, Ile, Lys, Leu, Met, Asn, Gln, Arg, Ser, Thr, Val, Trp or Tyr
  MISC_FEATURE (117)..(117): Xaa can be Phe, Asp, Glu, Phe, Gly, His, Ile, Lys, Met, Asn, Pro, Gln, Arg, Ser, Thr, Trp or Tyr
  MISC_FEATURE (118)..(118): Xaa can be Leu, Asp, Phe, Gly, His, Ile, Lys, Met, Asn, Pro, Gln, Arg, Ser, Thr, Val, Trp or Tyr
  MISC_FEATURE (119)..(119): Xaa can be Gly, Ala, Asp, Glu, Phe, His, Ile, Lys, Leu, Met, Asn, Pro, Gln, Arg, Ser, Thr, Val, Trp or Tyr
  MISC_FEATURE (120)..(120): Xaa can be Gly, Asp, Glu, Phe, His, Ile, Lys, Leu, Met, Asn, Pro, Gln, Arg, Ser, Thr, Val, Trp or Tyr
  MISC_FEATURE (121)..(121): Xaa can be Pro, Asp, Glu, Phe, Gly, His, Ile, Lys, Leu, Met, Asn, Gln, Arg, Ser, Thr, Val, Trp or Tyr
  MISC_FEATURE (122)..(122): Xaa can be Ser, Asp, Glu, Phe, Gly, His, Ile, Lys, Leu, Met, Asn, Pro, Gln, Arg, Thr, Val, Trp or Tyr
  MISC_FEATURE (123)..(123): Xaa can be Val, Ala, Ile, Met or Thr
  MISC_FEATURE (124)..(124): Xaa can be Phe, Asp, Glu, Leu, Arg, Ser, Trp or Tyr
  MISC_FEATURE (126)..(126): Xaa can be Phe, Glu, His, Leu, Gln, Arg, Trp or Tyr
  MISC_FEATURE (127)..(127): Xaa can be Pro or His
  MISC_FEATURE (128)..(128): Xaa can be Pro or Ala
  MISC_FEATURE (129)..(129): Xaa can be Lys, Asp, Glu, His or Tyr
  MISC_FEATURE (130)..(130): Xaa can be Pro, Gly or Val
  MISC_FEATURE (132)..(132): Xaa can be Asp, His, Gln or Tyr
  MISC_FEATURE (138)..(138): Xaa can be Arg, Glu or Tyr
  MISC_FEATURE (141)..(141): Xaa can be Glu, His, Ser or Tyr
  MISC_FEATURE (143)..(143): Xaa can be Glu, His, Ser or Tyr
  MISC_FEATURE (145)..(145): Xaa can be Val, Ala, Glu, Phe, Ile or Thr
  MISC_FEATURE (146)..(146): Xaa can be Val, Ala, Ile, Met or Thr
  MISC_FEATURE (147)..(147): Xaa can be Val, Ala, Asp, Glu, Phe, Gly, His, Ile, Lys, Leu, Met, Asn, Pro, Gln, Arg, Ser, Thr, Trp or Tyr
  MISC_FEATURE (148)..(148): Xaa can be Asp, Phe, Gly, His, Ile, Lys, Leu, Met, Pro, Gln, Arg, Ser, Thr, Val, Trp or Tyr
  MISC_FEATURE (149)..(149): Xaa can be Val, Ala, Ile, Met or Thr
  MISC_FEATURE (150)..(150): Xaa can be Ser, Asp, Glu, Phe, His, Ile, Lys, Leu, Met, Asn, Pro, Gln, Arg, Val, Trp or Tyr
  MISC_FEATURE (151)..(151): Xaa can be Gln, Asp, Glu, Phe, Gly, Ile, Lys, Leu, Met, Pro, Arg, Thr, Val or Trp
  MISC_FEATURE (152)..(152): Xaa can be Glu, Phe, Gly, His, Ile, Lys, Leu, Met, Asn, Pro, Arg, Ser, Thr, Val, Trp or Tyr
  MISC_FEATURE (153)..(153): Xaa can be Asp, Phe, Gly, His, Ile, Leu, Met, Pro, Gln, Arg, Ser, Thr, Trp or Tyr
  MISC_FEATURE (154)..(154): Xaa can be Pro, Ala, Asp, Glu, Phe, Gly, His, Ile, Lys, Leu, Met, Asn, Gln, Arg, Ser, Thr, Val, Trp or Tyr
  MISC_FEATURE (155)..(155): Xaa can be Glu, Asp, Phe, Gly, His, Ile, Lys, Leu, Met, Pro, Arg, Ser, Thr, Val, Trp or Tyr
  MISC_FEATURE (156)..(156): Xaa can be Val or Ile
  MISC_FEATURE (157)..(157): Xaa can be Gln, Asp, Glu, Phe, Gly, His, Ile, Leu, Met, Asn, Pro, Arg, Thr, Val, Trp or Tyr
  MISC_FEATURE (158)..(158): Xaa can be Phe, Leu, Trp
  MISC_FEATURE (159)..(159): Xaa can be Asn, Asp, Glu, Phe, Gly, His, Ile, Leu, Met, Pro, Arg, Ser, Thr, Val, Trp or Tyr
  MISC_FEATURE (161)..(161): Xaa can be Tyr, Asp, Glu, Gly, His, Ile, Lys, Leu, Met, Asn, Pro, Gln, Arg, Ser, Thr, Val or Trp
  MISC_FEATURE (163)..(163): Xaa can be Asp, Gly, Lys, Leu, Pro or Trp
  MISC_FEATURE (164)..(164): Xaa can be Gly, Asp, Glu, Lys, Asn, Pro, Gln or Tyr
  MISC_FEATURE (165)..(165): Xaa can be Val, Glu, Gly, Lys, Pro or Tyr
  MISC_FEATURE (166)..(166): Xaa can be Glu, Gly, His, Lys, Leu, Pro, Arg or Tyr
  MISC_FEATURE (167)..(167): Xaa can be Val, Asp, Glu, Leu, Asn, Gln, Thr or Tyr
  MISC_FEATURE (168)..(168): Xaa can be His, Asp, Glu, Lys, Gln, Trp or Tyr
  MISC_FEATURE (169)..(169): Xaa can be His, Asp, Glu, Lys, Gln, Trp or Tyr
  MISC_FEATURE (171)..(171): Xaa can be Lys, Asp, Glu or Tyr
  MISC_FEATURE (173)..(173): Xaa can be Lys, Asp, His, Leu, Asn or Trp
  MISC_FEATURE (174)..(174): Xaa can be Pro, Asp, Glu, Gly, His, Ile, Gln or Thr
  MISC_FEATURE (175)..(175): Xaa can be Arg, Asp, Glu, Thr or Tyr
  MISC_FEATURE (176)..(176): Xaa can be Glu, Phe, Gly, His, Ile, Leu, Met, Asn, Pro, Arg, Ser, Thr, Val, Trp or Tyr
  MISC_FEATURE (177)..(177): Xaa can be Glu, Phe, Gly, His, Ile, Lys, Leu, Met, Pro, Arg, Ser, Thr, Val, Trp or Tyr
  MISC_FEATURE (178)..(178): Xaa can be Gln, Asp, Glu, Phe, Gly, His, Ile, Met, Asn, Pro, Arg, Ser, Thr, Val, Trp or Tyr
  MISC_FEATURE (179)..(179): Xaa can be Phe, Ala, Asp, Glu, Gly, Ile, Lys, Leu, Met, Asn, Gln, Arg, Ser, Thr or Val
  MISC_FEATURE (180)..(180): Xaa can be Asn, Asp, Glu, Phe, Gly, His, Ile, Lys, Leu, Met, Pro, Gln, Arg, Ser, Thr, Val, Trp or Tyr
  MISC_FEATURE (181)..(181): Xaa can be Ser, Glu, Phe, His, Ile, Lys, Met, Gln, Arg, Trp or Tyr
  MISC_FEATURE (182)..(182): Xaa can be Thr, Ala, Asp, Glu, Phe, Gly, His, Ile, Lys, Leu, Met, Asn, Pro, Gln, Arg, Ser, Val, Trp or Tyr
  MISC_FEATURE (183)..(183): Xaa can be Tyr, Ala, Asp, Glu, Gly, His, Lys, Met, Asn, Pro, Gln, Arg, Ser, Thr, Val or Trp
  MISC_FEATURE (184)..(184): Xaa can be Arg, Asp, Glu, His or Tyr
  MISC_FEATURE (185)..(185): Xaa can be Val or Ile
  MISC_FEATURE (186)..(186): Xaa can be Val, Asp, Glu or Tyr
  MISC_FEATURE (187)..(187): Xaa can be Ser, Asp, His, Leu, Asn or Thr
  MISC_FEATURE (188)..(188): Xaa can be Val, Glu, Thr or Tyr
  MISC_FEATURE (196)..(196): Xaa can be Trp or Phe
  MISC_FEATURE (200)..(200): Xaa can be Lys, Glu or Gln
  MISC_FEATURE (201)..(201): Xaa can be Glu, His, Leu, Gln, Arg or Tyr
  MISC_FEATURE (203)..(203): Xaa can be Lys, Asp, Phe, Gly, His, Ile, Leu, Asn, Pro, Ser, Thr, Val, Trp or Tyr
  MISC_FEATURE (205)..(205): Xaa can be Lys, Asp, Phe, Gly, His, Ile, Pro, Ser, Thr, Val, Trp or Tyr
  MISC_FEATURE (206)..(206): Xaa can be Val or Ile
  MISC_FEATURE (207)..(207): Xaa can be Ser, Asp, Phe, Gly, His, Ile, Leu, Met, Pro, Arg, Thr, Val, Trp or Tyr
  MISC_FEATURE (208)..(208): Xaa can be Ser, Asp, Phe, Gly, His, Ile, Leu, Met, Pro, Arg, Thr, Val, Trp or Tyr
  MISC_FEATURE (209)..(209): Xaa can be Lys, Ile, Leu, Pro or Thr
  MISC_FEATURE (210)..(210): Xaa can be Gly, Asp, Glu, Phe, His, Ile, Lys, Leu, Met, Asn, Pro, Arg, Thr, Val, Trp or Tyr
  MISC_FEATURE (211)..(211): Xaa can be Leu, Ala, Asp, Glu, Phe, Gly, His, Ile, Lys, Met, Asn, Pro, Gln, Arg, Ser, Thr, Val, Trp or Tyr
  MISC_FEATURE (212)..(212): Xaa can be Pro, Asp, Glu, Phe, Gly, His, Ile, Lys, Leu, Met, Asn, Gln, Arg, Ser, Thr, Val, Trp or Tyr
  MISC_FEATURE (213)..(213): Xaa can be Ser, Glu, Phe, Gly, His, Ile, Leu, Met, Asn, Pro, Arg, Thr, Val, Trp or Tyr
  MISC_FEATURE (214)..(214): Xaa can be Ser, Asp, Phe, His, Ile, Leu, Met, Gln, Arg, Thr, Val, Trp or Tyr
  MISC_FEATURE (215)..(215): Xaa can be Ile, Ala, Asp, Glu, Phe, His, Lys, Leu, Met, Asn, Pro, Gln, Arg, Ser, Thr, Val, Trp or Tyr
  MISC_FEATURE (216)..(216): Xaa can be Glu, Phe, His, Ile, Leu, Met, Pro, Thr or Tyr
  MISC_FEATURE (217)..(217): Xaa can be Lys, Phe, Ile, Pro or Thr
  MISC_FEATURE (218)..(218): Xaa can be Thr, Asp, Phe, Gly, His, Ile, Leu, Met, Asn, Pro, Arg, Ser, Val, Trp or Tyr
  MISC_FEATURE (219)..(219): Xaa can be Ile, Glu, Lys or Tyr
  MISC_FEATURE (220)..(220): Xaa can be Ser, Glu, His or Asn
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGXXXXXCXXCXXXXXXXXXXXXLXXXXXKXTLMISXTPXVXCXXXXXXXXXXXXXXXWXVXXXXXXXAXTXXXXXXXXXXXXXXXXLTVLHQDXLNGXXYXCXXXXXXXXXXXXXXXXKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 110 - protein, 328 aa, Artificial Sequence
  IgG4 variant including an amino acid sequence
  MISC_FEATURE (104)..(104): Xaa can be no amino acid or Lys
  MISC_FEATURE (108)..(108): Xaa can be Pro or Gly
  MISC_FEATURE (115)..(115): Xaa can be Phe, Tyr or Ile
  MISC_FEATURE (116)..(116): Xaa can be Leu, Tyr, Ile or Asp
  MISC_FEATURE (117)..(117): )Xaa can be Gly, Ser or Ala
  MISC_FEATURE (118)..(118): Xaa can be Gly or Asp
  MISC_FEATURE (120)..(120): Xaa can be Ser, Asp, Glu, Asn, Gln or Thr
  MISC_FEATURE (121)..(121): Xaa can be Val, Ile or Met
  MISC_FEATURE (127)..(127): Xaa can be Lys, His or Tyr
  MISC_FEATURE (136)..(136): Xaa can be Arg or Tyr
  MISC_FEATURE (139)..(139): Xaa can be Glu, His or Tyr
  MISC_FEATURE (141)..(141): Xaa can be Thr or His
  MISC_FEATURE (145)..(145): Xaa can be Val, Ile, Thr or Tyr
  MISC_FEATURE (148)..(148): Xaa can be Ser, Asp or Glu
  MISC_FEATURE (149)..(149): Xaa can be Gln, Asp or Glu
  MISC_FEATURE (152)..(152): Xaa can be Pro or Gly
  MISC_FEATURE (153)..(153): Xaa can be Glu, Tyr, His, Arg or Ile
  MISC_FEATURE (155)..(155): Xaa can be Gln or Glu
  MISC_FEATURE (159)..(159): Xaa can be Tyr or Thr
  MISC_FEATURE (162)..(162): Xaa can be Gly, Asp or Glu
  MISC_FEATURE (164)..(164): Xaa can be Glu, Leu or His
  MISC_FEATURE (165)..(165): Xaa can be Val, Glu or Asp
  MISC_FEATURE (171)..(171): Xaa can be Lys or Asn
  MISC_FEATURE (174)..(174): Xaa can be Glu or Arg
  MISC_FEATURE (176)..(176): Xaa can be Gln or Glu
  MISC_FEATURE (185)..(185): Xaa can be Ser or Thr
  MISC_FEATURE (205)..(205): Xaa can be Ser, Gly or Ile
  MISC_FEATURE (207)..(207): Xaa can be Lys or Thr
  MISC_FEATURE (208)..(208): Xaa can be Gly or Asp
  MISC_FEATURE (209)..(209): Xaa can be Leu, Ala, Phe, Ile or Thr
  MISC_FEATURE (211)..(211): Xaa can be Ser, Leu, Tyr or Ile
  MISC_FEATURE (213)..(213): Xaa can be Ile, Asp, Glu, Asn, Gln or Thr
  MISC_FEATURE (214)..(214): Xaa can be Glu or Tyr
  MISC_FEATURE (215)..(215): Xaa can be Lys, Phe, Ile or Thr
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGXPPCXSCPAPEXXXXPXXFLFPPXPKDTLMISXTPXVXCVVXDVXXEDXXVXFNWXVDXVXXHNAKTXPRXEXFNSTYRVVXVLTVLHQDWLNGKEYKCKVXNXXXPXSXXXTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 111 - protein, 46 aa, Artificial Sequence
  IgG3 variant amino acid sequence having at least two amino acid modifications
RCPEPKSCDTPPPCPRCPEPKSCDTPPPCPRCPEPKSCDTPPPCPR

SEQ ID NO: 112 - protein, 4 aa, Artificial Sequence
  sequence linker
GFLG

(19) United States
(12) Patent Application Publication
(10) Pub. No.: US 2018/0360981 A1
(43) Pub. Date: Dec. 20, 2018

(54) NOVEL IMMUNOGLOBULIN VARIANTS

(71) Applicant: Xencor, Inc., Monrovia, CA (US)

(72) Inventors: Gregory Alan Lazar, Pacifica, CA (US); Bassil Dahiyat, Altadena, CA (US); Wei Dang, Pasadena, CA (US); John Desjarlais, Pasadena, CA (US); Sher Bahadur Karki, Santa Monica, CA (US); Omid Vafa, Monrovia, CA (US); Robert Hayes, Thousand Oaks, CA (US); Jost Vielmetter, Altadena, CA (US)

(21) Appl. No.: 15/883,006

(22) Filed: Jan. 29, 2018

Related U.S. Application Data
(63) Continuation of application No. 15/624,531, filed on Jun. 15, 2017, now abandoned.
Application No. 15/624,531 is a continuation of application No. 14/210,236, filed on Mar. 13, 2014, now abandoned.
Application No. 14/210,236 is a continuation-in-part of application No. 13/846,527, filed on Mar. 18, 2013, now Pat. No. 8,883,147.
Application No. 13/846,527 is a division of application No. 13/336,907, filed on Dec. 23, 2011, now Pat. No. 8,399,618.
Application No. 13/336,907 is a division of application No. 12/020,443, filed on Jan. 25, 2008, now Pat. No. 8,101,720.
Application No. 12/020,443 is a continuation-in-part of application No. 11/396,495, filed on Mar. 31, 2006, now abandoned.
Application No. 13/846,527 is a continuation-in-part of application No. 11/256,060, filed on Oct. 21, 2005, now abandoned.
Application No. 14/210,236 is a continuation-in-part of application No. 12/794,560, filed on Jun. 4, 2010, now Pat. No. 9,040,041.
Application No. 12/794,560 is a continuation of application No. 11/981,647, filed on Oct. 31, 2007, now abandoned.
Application No. 11/981,647 is a continuation of application No. 11/538,406, filed on Oct. 3, 2006, now abandoned.
Application No. 12/794,560 is a continuation of application No. 11/396,495, filed on Mar. 31, 2006, now abandoned.
Application No. 11/396,495 is a continuation-in-part of application No. 11/124,620, filed on May 5, 2005, now Pat. No. 8,188,231.
Application No. 11/124,620 is a continuation-in-part of application No. 10/822,231, filed on Mar. 26, 2004, now Pat. No. 7,317,091.
Application No. 10/822,231 is a continuation-in-part of application No. 10/672,280, filed on Sep. 26, 2003, now abandoned.
(60) Provisional application No. 61/801,168, filed on Mar. 15, 2013.

(51) Int. Cl. A61K 47/68; C07K 16/28; C07K 16/32

(52) U.S. Cl. CPC A61K 47/68; C07K 16/2887; C07K 2317/92; C07K 2317/732; C07K 2317/72; C07K 2317/734; C07K 2317/52; C07K 16/2818; C07K 16/2803; C07K 2317/53; C07K 16/32; C07K 2317/71

(57) ABSTRACT

The present invention relates to Fc variants with optimized Fc ligand binding properties, methods for their generation, Fc polypeptides comprising Fc variants with optimized Fc ligand binding properties, and methods for using same.

### CROSS-REFERENCE TO RELATED APPLICATIONS

This application is a continuation of U.S. patent application Ser. No. 15/624,531, filed Jun. 15, 2017 which is a continuation of U.S. patent application Ser. No. 14/210,236, filed Mar. 13, 2014 which claims priority to U.S. Provisional Application No. 61/801,168, filed Mar. 15, 2013. This application is also a continuation-in-part of U.S. patent application Ser. No. 13/846,527, filed Mar. 18, 2013, now U.S. Pat. No. 8,883,147 which is a division of Ser. No. 13/336,907, filed Dec. 23, 2011, now U.S. Pat. No. 8,399,618 which is a division of U.S. patent application Ser. No. 12/020,443 filed Jan. 25, 2008, now U.S. Pat. No. 8,101,720 which is a continuation-in-part of U.S. patent application Ser. No. 11/396,495, filed Mar. 31, 2006, now abandoned; and said U.S. patent application Ser. No. 13/846,527, filed Mar. 18, 2013 is also a continuation-in-part of U.S. patent application Ser. No. 11/256,060, filed Oct. 21, 2005, now abandoned. U.S. patent application Ser. No. 14/210,236, filed Mar. 13, 2014 is also a continuation-in-part of U.S. patent application Ser. No. 12/794,560, filed Jun. 4, 2010, which is a continuation of U.S. patent application Ser. No. 11/981,647, filed Oct. 31, 2007, now abandoned, which is a continuation of U.S. patent application Ser. No. 11/538,406, filed Oct. 3, 2006, now abandoned; and said U.S. patent application Ser. No. 12/794,560, filed Jun. 4, 2010 is also a continuation of U.S. patent application Ser. No. 11/396,495, filed Mar. 31, 2006, now abandoned, which is a continuation-in-part of U.S. patent application Ser. No. 11/124,620, filed May 5, 2005, now U.S. Pat. No. 8,188,231, which is a continuation-in-part of U.S. patent application Ser. No. 10/822,231, filed Mar. 26, 2004, now U.S. Pat. No. 7,317,091, which is a continuation-in-part of U.S. patent application Ser. No. 10/672,280, filed Sep. 26, 2003, now abandoned, each of which is incorporated herein by reference in its entirety for all purposes. In particular, the sequence listings, including the sequence numbers as unique to each of the aforementioned applications, with their corresponding sequences, description, and portions of specification referencing or describing same are incorporated herein by reference in their entireties.

### SEQUENCE LISTING

The instant application contains a Sequence Listing which has been submitted electronically in ASCII format and is hereby incorporated by reference in its entirety. Said ASCII copy, created on Jan. 29, 2018, is named 067461-5166-U503_SL_ST25.txt and is 449,761 bytes in size.

### FIELD OF THE INVENTION

The present invention relates to novel immunoglobulin insertions, deletions, and substitutions that provide optimized effector function properties, engineering methods for their generation, and their application, particularly for therapeutic purposes.

### BACKGROUND OF THE INVENTION

Antibodies are immunological proteins that bind a specific antigen. Generally, antibodies are specific for targets, have the ability to mediate immune effector mechanisms, and have a long half-life in serum. Such properties make antibodies powerful therapeutics. Monoclonal antibodies are used therapeutically for the treatment of a variety of conditions including cancer, infectious disease, autoimmune disease, and inflammatory disorders. In addition to antibodies, an antibody-like protein that is finding an expanding role in research and therapy is the Fc fusion (Chamow et al., 1996, Trends Biotechnol 14:52-60; Ashkenazi et al., 1997, Curr Opin Immunol 9:195-200, incorporated by reference). There are currently over twenty antibody and Fc fusion products on the market and hundreds in development.

Antibodies have found widespread application in oncology, particularly for targeting cellular antigens selectively expressed on tumor cells with the goal of cell destruction. There are a number of mechanisms by which antibodies destroy tumor cells, including anti-proliferation via blockage of needed growth pathways, intracellular signaling leading to apoptosis, enhanced down regulation and/or turnover of receptors, CDC, ADCC, ADCP, and promotion of an adaptive immune response (Cragg et al., 1999, Curr Opin Immunol 11:541-547; Glennie et al., 2000, Immunol Today 21:403-410, both hereby entirely incorporated by reference). Anti-tumor efficacy may be due to a combination of these mechanisms, and their relative importance in clinical therapy appears to be cancer dependent.

Despite this arsenal of anti-tumor weapons, the potency of antibodies as anti-cancer agents is unsatisfactory, particularly given their high cost. Patient tumor response data show that monoclonal antibodies provide only a small improvement in therapeutic success over normal single-agent cytotoxic chemotherapeutics. For example, just half of all relapsed low-grade non-Hodgkin's lymphoma patients respond to the anti-CD20 antibody rituximab (McLaughlin et al., 1998, J Clin Oncol 16:2825-2833, hereby entirely incorporated by reference). Of 166 clinical patients, 6% showed a complete response and 42% showed a partial response, with median response duration of approximately 12 months. Trastuzumab (Herceptin™, Genentech), an anti-HER2/neu antibody for treatment of metastatic breast cancer, has less efficacy. The overall response rate using trastuzumab for the 222 patients tested was only 15%, with 8 complete and 26 partial responses and a median response duration and survival of 9 to 13 months (Cobleigh et al., 1999, J Clin Oncol 17:2639-2648, hereby entirely incorporated by reference). Currently for anticancer therapy, any small improvement in mortality rate defines success. Thus, there is a significant need to enhance the capacity of antibodies to destroy targeted cancer cells.

One potential way to improve the activity of anti-cancer therapeutics is to optimize their affinity and/or selectivity for Fc gamma receptors (FcγRs). Because all FcγRs interact with the same binding site on Fc, and because of the high homology among the FcγRs, obtaining variants that selectively increase or reduce FcγR affinity is a major challenge. Thus, there is a need to make Fc variants that selectively increase or reduce FcγR affinity.

Uchida et al. (J Exp Med v 199, p 1659, 21 Jun. 2004) found that anti-CD20 monoclonal antibody depletion of B cells in mice is greatly impaired in FcR common gamma chain knockouts. This impairment is 60-70% for blood B cells and is complete for splenic B cells. Depletion is not significantly impaired in their model for FcγRII or FcγRIII knockouts, and is only somewhat depleted in FcγRI knockouts, showing that either activating receptor RIII or RI is sufficient. It is noted that macrophages possess both RI and RIII, whereas natural killer cells possess only RIII. These findings and further experiments with this model in genetic and pharmacological knockouts of specific effector cell lineages demonstrates that macrophage activity was the impaired component that caused loss of anti-CD20 cytolysis, and that neither complement nor NK cells were responsible for this abrogation. Taken together, these results provide strong support for the hypothesis that macrophages are a key effector cell type for anti-CD20 therapy and that depletion of B cells likely involves BOTH phagocytic and secreted cytolytic factors. Also, depletion of splenic B-cells, clearly less rapid and requiring more potency than blood B cells, is achieved by anti-CD20 antibodies that effectively recruit macrophages, and splenic B cell depletion is greatly impaired by disruption of macrophage activity or FcR common gamma chain knockout.

A substantial obstacle to engineering anti-CD20 antibodies with the desired properties is the difficulty in predicting what amino acid modifications, out of the enormous number of possibilities, will achieve the desired goals, coupled with the inefficient production and screening methods for antibodies. Indeed, one of the principle reasons for the incomplete success of the prior art is that approaches to Fc engineering have thus far involved hit-or-miss methods such as alanine scans or production of glycoforms using different expression strains.

In summary, there is a need for antibodies with enhanced therapeutic properties. Despite such widespread use, anti-CD20 antibodies are not optimized for clinical use. Two significant deficiencies of antibodies are their suboptimal anticancer potency and their demanding production requirements. In these studies, the Fc modifications that were made were fully or partly random in hopes of obtaining variants with favorable properties. These deficiencies are addressed by the present invention. FcγRFcγRIn contrast to antibody therapeutics and indications wherein effector functions contribute to clinical efficacy, for some antibodies and clinical applications it may be favorable to reduce or eliminate binding to one or more FcγRs, or reduce or eliminate one or more FcγR- or complement-mediated effector functions including but not limited to ADCC, ADCP, and/or CDC. This is often the case for therapeutic antibodies whose mechanism of action involves blocking or antagonism but not killing of the cells bearing target antigen. In these cases depletion of target cells is undesirable and can be considered a side effect. For example, the ability of anti-CD4 antibodies to block CD4 receptors on T cells makes them effective anti-inflammatories, yet their ability to recruit FcγR receptors also directs immune attack against the target cells, resulting in T cell depletion (Reddy et al., 2000, J Immunol 164:1925-1933, incorporated entirely by reference). Effector function may also be a problem for radiolabeled antibodies, referred to as radioconjugates, and antibodies conjugated to toxins, referred to as immunotoxins. These drugs can be used to destroy cancer cells, but the recruitment of immune cells via Fc interaction with FcγRs brings healthy immune cells in proximity to the deadly payload (radiation or toxin), resulting in depletion of normal lymphoid tissue along with targeted cancer cells (Hutchins et al., 1995, Proc Natl Acad Sci USA 92:11980-11984; White et al., 2001, Annu Rev Med 52:125-145, both incorporated entirely by reference). What is needed is a general and robust means to completely ablate all FcγR binding and FcγR- and complement-mediated effector functions. These and other needs are addressed by the present invention.

### SUMMARY OF THE INVENTION

The present invention is directed to an Fc variant of a parent Fc polypeptide, wherein said Fc variant comprises amino modifications, which can comprise independently or in combination amino acid insertion(s), amino acid deletion(s) and/or amino acid substitutions described herein.

In one aspect of the invention, the Fc variant of the invention comprises an amino acid insertion after a position selected from the group consisting of 233, 234, 235, 236, and 237, wherein numbering is according to the EU index. The Fc variant may comprise an amino acid substitution in the Fc region. In one embodiment, said substitution occurs at a position selected from the group consisting of 235, 236, 237, 239, 243, 267, 299, 325, 328, 330, 332, and 328, wherein numbering is according to the EU index. In a preferred embodiment, said substitution is selected from the group consisting of 235G, 236A, 236R, 237K, 239D, 239E, 243L, 267D, 267E, 299T, 325L, 325A, 328F, 328R, 330Y, 330L, 332D, 332E. In another embodiment, said substitution occurs at a position selected from the group consisting of 234, 235, 236, 239, 243, 247, 255, 267, 268, 270, 280, 292, 293, 295, 298, 300, 305, 324, 326, 327, 328, 330, 332, 333, 334, 392, 396, and 421, wherein numbering is according to the EU index. In a preferred embodiment, said substitution is selected from the group consisting of 234G, 234I, 235D, 235E, 235I, 235Y, 236A, 236S, 239D, 239E, 243L, 247L, 255L, 267D, 267E, 267Q, 268D, 268E, 270E, 280H, 280Q, 280Y, 292P, 293R, 295E, 298A, 298T, 298N, 300L, 3051, 324G, 324I, 326A, 326D, 326E, 326W, 326Y, 327H, 328A, 328F, 328I, 330I, 330L, 330Y, A330V, 332D, 332E, 333A, 333S, 334A, 334L, 392T, 396L, and 421K.

In another aspect of the invention, the Fc variant of the invention comprises an amino acid deletion at a position selected from the group consisting of 233, 234, 235, 236, and 237, wherein numbering is according to the EU index. The Fc variant may additionally comprise an amino acid substitution in the Fc region. In one embodiment, said substitution occurs at a position selected from the group consisting of 235, 236, 237, 325, and 328, wherein numbering is according to the EU index. In a preferred embodiment, said substitution is selected from the group consisting of 235G, 236R, 237K, 325L, 325A, and 328R. In another embodiment, said substitution occurs at a position selected from the group consisting of 234, 235, 236, 239, 243, 247, 255, 267, 268, 270, 280, 292, 293, 295, 298, 300, 305, 324, 326, 327, 328, 330, 332, 333, 334, 392, 396, and 421, wherein numbering is according to the EU index. In a preferred embodiment, said substitution is selected from the group consisting of 234G, 234I, 235D, 235E, 235I, 235Y, 236A, 236S, 239D, 239E, 243L, 247L, 255L, 267D, 267E, 267Q, 268D, 268E, 270E, 280H, 280Q, 280Y, 292P, 293R, 295E, 298A, 298T, 298N, 300L, 3051, 324G, 324I, 326A, 326D, 326E, 326W, 326Y, 327H, 328A, 328F, 328I, 330I, 330L, 330Y, A330V, 332D, 332E, 333A, 333S, 334A, 334L, 392T, 396L, and 421K.

The present application is directed to IgG1, IgG2, IgG3, and IgG4 variants. Certain variants include isotopic amino acid modifications between IgG1, IgG2, IgG3, and IgG4. The variations can include isotopic modifications between in at least 2 domains, 3 domains, or 4 domains. Exchange domains can be CH1, CH2, hinge, and CH3 domains, CH1, CH2, and CH3 domains, or CH2 and CH3 domains.

Alternatively, certain specific modifications can be made to IgG1, IgG2, IgG3, or IgG4 scaffolds that are not found in any other IgG subclass. In certain embodiments, the variations can occur within only the Fc region of the IgG subclass, or only within one or more specific domains.

In additional aspects, IgG1, IgG2, IgG3, and IgG4 variants that exhibit altered binding to an FcγR or enhances effector function as compared to native IgG polypeptides can be designed. For example, altered binding to an FcγR such as FcγRI, FcγRIIa, FcγRIIb, FcγRIIc, or FcγRIIIa can be designed. Alternatively, one or more effector functions (e.g. ADCC, ADCP, and CDC) can be designed.

In one aspect, the present application is directed to IgG2, IgG3, or IgG4 variants with one or more isotypic substitutions. In an embodiment, of such variants, the IgG1, IgG2, IgG3, or IgG4 variant including an amino acid sequence having the formula:

(SED ID NO: 87)
ASTKGPSVFPLAP-X(131)-S-X(133)-STS-X(137)-X(138)-
TAALGCLVKDYFPEPVTVSWNSGALTSGVHTFPAVLQSSGLYSLSSVVTVPSS-
X(192)-X(193)-GT-X(196)-TY-X(199)-CNV-X(203)-HKPSNTKVDK-X(214)-VE-X(217)-
K-X(219)-X(220)-X(221)-X(222)-X(223)-X(224)-X(225)-CP-X(228)-CPAP-X(233)-
X(234)-X(235)-X(236)-GPSVFLFPPKPKDTLMISRTPEVTCVVVDVS-X(268)-EDPEV-
X(274)-F-X(276)-WYVDGVEVHNAKTKPREEQ-X(296)-NST-X(300)-RVVSVLTV-
X(309)-HQDWLNGKEYKCKVSNK-X(327)-LP-X(330)-X(331)-IEKTISK-X(339)-
KGQPREPQVYTLPPS-X(355)-X(356)-E-X(358)-TKNQVSLTCLVKGFYPSDIAVEWES-
X(384)-GQPENNY-X(392)-TTPP-X(397)-LDSDGSFFLYS-X(409)-LTVDKSRWQ-
X(419)-GN-X(422)-FSCSVMHEALHN-X(435)-X(436)-TQKSLSLS-X(445)-GK,

wherein
—X(131)- is selected from the group consisting of C and S;
—X(133)- is selected from the group consisting of R and K;
—X(137)- is selected from the group consisting of E and G;
—X(138)- is selected from the group consisting of S and G;
—X(192)- is selected from the group consisting of N and S;
—X(193)- is selected from the group consisting of F and L;
—X(196)- is selected from the group consisting of Q and K;
—X(199)- is selected from the group consisting of T and I;
—X(203)- is selected from the group consisting of D and N;
—X(214)- is selected from the group consisting of T, K and R;
—X(217)- is selected from the group consisting of R, P, L and S;
—X(219)- is selected from the group consisting of C, S, T and Y;
—X(220)- is selected from the group consisting of C, P and G;
—X(221)- is selected from the group consisting of no amino acid, D, L and the sequence LGD;
—X(222)- is selected from the group consisting of V, K, T and no amino acid;
—X(223)- is selected from the group consisting of no amino acid and T;
—X(224)- is selected from the group consisting of E, H and P;
—X(225)- is selected from the group consisting of no amino acid, T and P;
—X(228)- is selected from the group consisting of P, S, R, and the sequence

(SEQ ID NO: 111)

RCPEPKSCDTPPPCPRCPEPKSCDTPPPCPRCPEPKSCDTPPPCPR;

—X(233)- is selected from the group consisting of P and E;
—X(234)- is selected from the group consisting of V, L and F;
—X(235)- is selected from the group consisting of A and L;
—X(236)- is selected from the group consisting of no amino acid and G;
—X(268)- is selected from the group consisting of H and Q;
—X(274)- is selected from the group consisting of Q and K;
—X(276)- is selected from the group consisting of N and K;
—X(296)- is selected from the group consisting of F and Y;
—X(300)- is selected from the group consisting of F and Y;
—X(309)- is selected from the group consisting of V and L;
—X(327)- is selected from the group consisting of G and A;
—X(330)- is selected from the group consisting of A and S;
—X(331)- is selected from the group consisting of P and S;
—X(339)- is selected from the group consisting of T and A;
—X(355)- is selected from the group consisting of R and Q;
—X(356)- is selected from the group consisting of E and D;
—X(358)- is selected from the group consisting of M and L;
—X(384)- is selected from the group consisting of N and S;
—X(392)- is selected from the group consisting of K and N;
—X(397)- is selected from the group consisting of M and V;
—X(409)- is selected from the group consisting of K and R;
—X(419)- is selected from the group consisting of Q and E;
—X(422)- is selected from the group consisting of V and I;
—X(435)- is selected from the group consisting of H and R;
—X(436)- is selected from the group consisting of Y and F; and
—X(445)- is selected from the group consisting of P and L.

Variants of the formula can have at least 1, 2, 3, 4, 5, 6, 7, 8, 9, 10 or more amino acid modifications as compared to an amino acid sequence including SEQ ID NO:11, SEQ ID NO:12, or SEQ ID NO:4. In a further embodiment, at least two of the modifications can be in different domains, at least three modifications can be in different domains, or at least four modifications can be in different domains.

In a further aspect, the present application is directed to a IgG2, IgG3, or IgG4 variant including at least 1, 2, 3, 4, 5, 6, 7, 8, 9, 10 or more amino acid modifications as compared to an amino acid sequence including SEQ ID NO:11, SEQ ID NO:12, or SEQ ID NO:4. The modification can be at one or more positions selected from among positions 131, 133, 137, 138, 192, 193, 196, 199, 203, 214, 217, 219, 221, 222, 223, 224, 225, 226, 227, 228, 229, 230, 233, 234, 235, 236, 268, 274, 296, 300, 309, 327, 330, 335, 339, 356, 358, 384, 392, 397, 409, 419, 422, 435, 436 and 445. In further embodiments, at least two of the modifications can be in different domains, at least three modifications can be in different domains, or at least four modifications can be in different domains.

In another aspect, the present application is directed to an IgG2 variant including an amino acid sequence having the formula:

(SED ID NO: 88)
ASTKGPSVFPLAP-X(131)-S-X(133)-STS-X(137)-X(138)-
TAALGCLVKDYFPEPVTVSWNSGALTSGVHTFPAVLQSSGLYSLSSVVTVPSS-
X(192)-X(193)-GTQTY-X(199)-CNV-X(203)-HKPSNTKVDK-X(214)-VE-X(217)-K-
X(219)-C-X(221)-X(222)-X(223)-X(224)-X(225)-CPPCPAP-X(233)-X(234)-X(235)-
X(236)-GPSVFLFPPKPKDTLMISRTPEVTCVVVDVSHEDPEV-X(274)-
FNWYVDGVEVHNAKTKPREEQ-X(296)-NST-X(300)-RVVSVLTV-X(309)-
HQDWLNGKEYKCKVSNK-X(327)-LPAPIEKTISK-X(339)-KGQPREPQVYTLPPSR-
X(356)-E-X(358)-TKNQVSLTCLVKGFYPSDIAVEWESNGQPENNYKTTPP-X(397)-
LDSDGSFFLYSKLTVDKSRWQQGNVFSCSVMHEALHNHYTQKSLSLSPGK,

wherein
X(131) is selected from the group consisting of C and S;
X(133) is selected from the group consisting of R and K;
X(137) is selected from the group consisting of E and G;
X(138) is selected from the group consisting of S and G;
X(192) is selected from the group consisting of N and S;
X(193) is selected from the group consisting of F and L;
X(199) is selected from the group consisting of T and I;
X(203) is selected from the group consisting of D and N;
X(214) is selected from the group consisting of T and K;
X(217) is selected from the group consisting of R and P;
X(219) is selected from the group consisting of C and S;
X(221) is selected from the group consisting of no amino acid and D;
X(222) is selected from the group consisting of V and K;
X(223) is selected from the group consisting of no amino acid and T;
X(224) is selected from the group consisting of E and H;
X(225) is selected from the group consisting of no amino acid and T;
X(233) is selected from the group consisting of P and E;
X(234) is selected from the group consisting of V and L;
X(235) is selected from the group consisting of A and L;
X(236) is selected from the group consisting of no amino acid and G;
X(274) is selected from the group consisting of Q and K;
X(296) is selected from the group consisting of F and Y;
X(300) is selected from the group consisting of F and Y;
X(309) is selected from the group consisting of V and L;
X(327) is selected from the group consisting of G and A;
X(339) is selected from the group consisting of T and A;
X(356) is selected from the group consisting of E and D;
X(358) is selected from the group consisting of M and L; and
X(397) is selected from the group consisting of M and V.

In various embodiments, the formula has at least 1, 2, 3, 4, 5, 6, 7, 8, 9, 10 or more amino acid modifications as compared to an amino acid sequence including SEQ ID NO:11. In additional embodiments, at least 2, 3, or 4 of the modifications are in different domains.

In another aspect, the present application is directed to an IgG2 variant including two or more amino acid modifications as compared to SEQ ID NO:11. The modification can be selected from among C131S, R133K, E137G, S138G, N192S, F193L, T199I, D203N, T214K, R217P, C219S, insertion of 221D, V222K, insertion of 223T, E224H, insertion of 225T, P233E, V234L, A235L, insertion of 236G, Q274K, F296Y, F300Y, V309L, G327A, T339A, E356D, M358L, and M397V. In various embodiments, the formula has at least 1, 2, 3, 4, 5, 6, 7, 8, 9, 10 or more amino acid modifications as compared to an amino acid sequence including SEQ ID NO:11. In additional embodiments, at least 2, 3, or 4 of the modifications are in different domains.

In a further variation, the present application is directed to an IgG2 variant including an amino acid sequence having the formula:

(SED ID NO: 89)

-ASTKGPSVFPLAP-X(131)-S-X(133)-STS-X(137)-X(138)-
TAALGCLVKDYFPEPVTVSWNSGALTSGVHTFPAVLQSSGLYSLSSVVTV
PSS-X(192)-X(193)-GT-X(196)-TY-X(199)-CNV-X(203)-
HKPSNTKVDK-X(214)-VE-X(217)-K-X(219)-X(220)-
X(221)-X(222)-X(223)-X(224)-X(225)-C-X(227)-
X(228)-C-X(230)-X(231)-X(232)-X(233)-X(234)-
X(235)-X(236)-X(237)-X(238)-X(239)-X(240)-X(241)-
L-X(243)-X(244)-X(245)-X(246)-X(247)-K-X(249)-
TLMIS-X(255)-TP-X(258)-V-X(260)-C-X(262)-X(263)-
X(264)-X(265)-X(266)-X(267)-X(268)-X(269)-X(270)-
X(271)-X(272)-X(273)-X(274)-X(275)-X(276)-W-
X(278)-V-X(280)-X(281)-X(282)-X(283)-X(284)-
X(285)-X(286)-A-X(288)-T-X(290)-X(291)-X(292)-
X(293)-X(294)-X(295)-X(296)-X(297)-X(298)-X(299)-
X(300)-X(301)-X(302)-X(303)-X(304)-X(305)-LTV-
X(309)-HQD-X(313)-LNG-X(317)-X(318)-Y-X(320)-C-
X(322)-X(323)-X(324)-X(325)-X(326)-X(327)-X(328)-
X(329)-X(330)-X(331)-X(332)-X(333)-X(334)-X(335)-
X(336)-X(337)-K-X(339)-KGQPREPQVYTLPPS-X(355)-
X(356)-E-X(358)-TKNQVSLTCLVKGFYPSDIAVEWES-X(384)-
GQPENNY-X(392)-TTPP-X(397)-LDSDGSFFLYS-X(409)-LTVD
KSRWQ-X(419)-GN-X(422)-FSCSVMHEALHN-X(435)-X(436)-
TQKSLSLS-X(445)-GK-,

wherein
—X(131)- is selected from the group consisting of C and S;
—X(133)- is selected from the group consisting of R and K;
—X(137)- is selected from the group consisting of E and G;
—X(138)- is selected from the group consisting of S and G;
—X(192)- is selected from the group consisting of N and S;
—X(193)- is selected from the group consisting of F and L;
—X(196)- is selected from the group consisting of Q and K;
—X(199)- is selected from the group consisting of T and I;
—X(203)- is selected from the group consisting of D and N;
—X(214)- is selected from the group consisting of T, K and R;
—X(217)- is selected from the group consisting of R, P, L and S;
—X(219)- is selected from the group consisting of C, S, T and Y;
—X(220)- is selected from the group consisting of C, P and G;
—X(221)- is selected from the group consisting of no amino acid, D, K, L, Y and the sequence LGD;
—X(222)- is selected from the group consisting of V, K, T, no amino acid, E and Y;
—X(223)- is selected from the group consisting of no amino acid, T, E and K;
—X(224)- is selected from the group consisting of E, H, P and Y;
—X(225)- is selected from the group consisting of no amino acid, T, P, E, K and W;
—X(227)- is selected from the group consisting of P, E, G, K and Y;
—X(228)- is selected from the group consisting of P, S, R, E, G, K, Y, and the sequence

(SEQ ID NO: 111)

RCPEPKSCDTPPPCPRCPEPKSCDTPPPCPRCPEPKSCDTPPPCPR;

—X(230)- is selected from the group consisting of P, A, E, G and Y;
—X(231)- is selected from the group consisting of A, E, G, K, P and Y;
—X(232)- is selected from the group consisting of P, E, G, K and Y;
—X(233)- is selected from the group consisting of P, E, A, D, F, G, H, I, K, L, M, N, Q, R, S, T, V, W and Y;
—X(234)- is selected from the group consisting of V, L, F, D, E, F, G, H, I, K, M, N, P, Q, R, S, T, W and Y;
—X(235)- is selected from the group consisting of A, L, D, F, G, H, I, K, M, N, P, Q, R, S, T, V, W, and Y;
—X(236)- is selected from the group consisting of no amino acid, G, A, D, E, F, H, I, K, L, M, N, P, Q, R, S, T, V, W and Y;
—X(237)- is selected from the group consisting of G, D, E, F, H, I, K, L, M, N, P, Q, R, S, T, V, W and Y;
—X(238)- is selected from the group consisting of P, D, E, F, G, H, I, K, L, M, N, Q, R, S, T, V, W and Y;
—X(239)- is selected from the group consisting of S, D, E, F, G, H, I, K, L, M, N, P, Q, R, T, V, W and Y;
—X(240)- is selected from the group consisting of V, A, I, M and T;
—X(241)- is selected from the group consisting of F, D, E, L, R, S, W and Y;
—X(243)- is selected from the group consisting of F, E, H, L, Q, R, W, and Y;
—X(244)- is selected from the group consisting of P and H;
—X(245)- is selected from the group consisting of P and A;
—X(246)- is selected from the group consisting of K, D, E, H and Y;
—X(247)- is selected from the group consisting of P, G and V;
—X(249)- is selected from the group consisting of D, H, Q and Y;
—X(255)- is selected from the group consisting of R, E, and Y;
—X(258)- is selected from the group consisting of E, H, S and Y;
—X(260)- is selected from the group consisting of T, D, E, H and Y;
—X(262)- is selected from the group consisting of V, A, E, F, I and T;
—X(263)- is selected from the group consisting of V, A, I, M and T;
—X(264)- is selected from the group consisting of V, A, D, E, F, G, H, I, K, L, M, N, P, Q, R, S, T, W, and Y;
—X(265)- is selected from the group consisting of D, F, G, H, I, K, L, M, P, Q, R, S, T, V, W and Y;
—X(266)- is selected from the group consisting of V, A, I, M and T;
—X(267)- is selected from the group consisting of S, D, E, F, H, I, K, L, M, N, P, Q, R, V, W and Y;
—X(268)- is selected from the group consisting of H, Q, D, E, F, G, I, K, L, M, P, R, T, V and W;
—X(269)- is selected from the group consisting of E, F, G, H, I, K, L, M, N, P, R, S, T, V, W and Y;
—X(270)- is selected from the group consisting of D, F, G, H, I, L, M, P, Q, R, S, T, W and Y;
—X(271)- is selected from the group consisting of P, A, D, E, F, G, H, I, K, L, M, N, Q, R, S, T, V, W and Y;
—X(272)- is selected from the group consisting of E, D, F, G, H, I, K, L, M, P, R, S, T, V, W and Y;
—X(273)- is selected from the group consisting of V and I;
—X(274)- is selected from the group consisting of Q, K, D, E, F, G, H, I, L, M, N, P, R, T, V, W and Y;
—X(275)- is selected from the group consisting of F, L and W;
—X(276)- is selected from the group consisting of N, K, D, E, F, G, H, I, L, M, P, R, S, T, V, W and Y;
—X(278)- is selected from the group consisting of Y, D, E, G, H, I, K, L, M, N, P, Q, R, S, T, V and W;
—X(280)- is selected from the group consisting of D, G, K, L, P and W;
—X(281)- is selected from the group consisting of G, D, E, K, N, P, Q and Y;
—X(282)- is selected from the group consisting of V, E, G, K, P and Y;
—X(283)- is selected from the group consisting of E, G, H, K, L, P, R and Y;
—X(284)- is selected from the group consisting of V, D, E, L, N, Q, T and Y;
—X(285)- is selected from the group consisting of H, D, E, K, Q, W and Y;
—X(286)- is selected from the group consisting of N, E, G, P and Y;
—X(288)- is selected from the group consisting of K, D, E and Y;
—X(290)- is selected from the group consisting of K, D, H, L, N and W;
—X(291)- is selected from the group consisting of P, D, E, G, H, I, Q and T;
—X(292)- is selected from the group consisting of R, D, E, T and Y;
—X(293)- is selected from the group consisting of E, F, G, H, I, L, M, N, P, R, S, T, V, W and Y;
—X(294)- is selected from the group consisting of E, F, G, H, I, K, L, M, P, R, S, T, V, W and Y;
—X(295)- is selected from the group consisting of Q, D, E, F, G, H, I, M, N, P, R, S, T, V, W and Y;
—X(296)- is selected from the group consisting of F, Y, A, D, E, G, I, K, L, M, N, Q, R, S, T and V;
—X(297)- is selected from the group consisting of N, D, E, F, G, H, I, K, L, M, P, Q, R, S, T, V, W and Y;
—X(298)- is selected from the group consisting of S, E, F, H, I, K, M, Q, R, W and Y;
—X(299)- is selected from the group consisting of T, A, D, E, F, G, H, I, K, L, M, N, P, Q, R, S, V, W and Y;
—X(300)- is selected from the group consisting of F, Y, A, D, E, G, H, K, M, N, P, Q, R, S, T, V and W;
—X(301)- is selected from the group consisting of R, D, E, H and Y;
—X(302)- is selected from the group consisting of V and I;
—X(303)- is selected from the group consisting of V, D, E and Y;
—X(304)- is selected from the group consisting of S, D, H, L, N and T;
—X(305)- is selected from the group consisting of V, E, T and Y;
—X(309)- is selected from the group consisting of V and L;
—X(313)- is selected from the group consisting of W and F;
—X(317)- is selected from the group consisting of K, E and Q;
—X(318)- is selected from the group consisting of E, H, L, Q, R and Y;
—X(320)- is selected from the group consisting of K, D, F, G, H, I, L, N, P, S, T, V, W and Y;
—X(322)- is selected from the group consisting of K, D, F, G, H, I, P, S, T, V, W and Y;
—X(323)- is selected from the group consisting of V and I;
—X(324)- is selected from the group consisting of S, D, F, G, H, I, L, M, P, R, T, V, W and Y;
—X(325)- is selected from the group consisting of N, A, D, E, F, G, H, I, K, L, M, P, Q, R, S, T, V, W and Y;
—X(326)- is selected from the group consisting of K, I, L, P and T;
—X(327)- is selected from the group consisting of G, A, D, E, F, H, I, K, L, M, N, P, R, T, V, W and Y;
—X(328)- is selected from the group consisting of L, A, D, E, F, G, H, I, K, M, N, P, Q, R, S, T, V, W and Y;
—X(329)- is selected from the group consisting of P, D, E, F, G, H, I, K, L, M, N, Q, R, S, T, V, W and Y;
—X(330)- is selected from the group consisting of A, S, E, F, G, H, I, L, M, N, P, R, T, V, W and Y;
—X(331)- is selected from the group consisting of P, S, D, F, H, I, L, M, Q, R, T, V, W and Y;
—X(332)- is selected from the group consisting of I, A, D, E, F, H, K, L, M, N, P, Q, R, S, T, V, W and Y;
—X(333)- is selected from the group consisting of E, F, H, I, L, M, P, T and Y;
—X(334)- is selected from the group consisting of K, F, I, P and T;
—X(335)- is selected from the group consisting of T, D, F, G, H, I, L, M, N, P, R, S, V, W and Y;
—X(336)- is selected from the group consisting of I, E, K and Y;
—X(337)- is selected from the group consisting of S, E, H and N;
—X(339)- is selected from the group consisting of T and A;
—X(355)- is selected from the group consisting of R and Q;
—X(356)- is selected from the group consisting of E and D;
—X(358)- is selected from the group consisting of M and L;
—X(384)- is selected from the group consisting of N and S;
—X(392)- is selected from the group consisting of K and N;
—X(397)- is selected from the group consisting of M and V;
—X(409)- is selected from the group consisting of K and R;
—X(419)- is selected from the group consisting of Q and E;
—X(422)- is selected from the group consisting of V and I;
—X(435)- is selected from the group consisting of H and R;
—X(436)- is selected from the group consisting of Y and F; and
—X(445)- is selected from the group consisting of P and L.

In certain variations, a first modification is selected from among C131S, R133K, E137G, S138G, N192S, F193L, Q196K, T199I, D203N, T214K, T214R, R217P, R217L, R217S, C219S, C219T, C219Y, C220P, C220G, insertion of 221D, insertion of 221L, insertion of 221LGD, V222K, V222T, deletion of V222, insertion of 223T, E224H, E224P, insertion of 225T, insertion of 225P. P228R, substitution of P228 with

(SEQ ID NO: 111)

RCPEPKSCDTPPPCPRCPEPKSCDTPPPCPRCPEPKSCDTPPPCPR,

P228S, P233E, V234L, V234F, A235L, insertion of 236G, H268Q, Q274K, N276K, F296Y, F300Y, V309L, G327A, A330S, P331S, T339A, R355Q, E356D, M358L, N384S, K392N, M397V, K409R, Q419E, V422I, H435R, Y436F, and P445L. In a further variation, a second modification is selected from among 221K, 221Y, 222E, 222Y, 223E, 223K, 224Y, 225E, 225K, 225W, 227E, 227G, 227K, 227Y, 228E, 228G, 228K, 228Y, 230A, 230E, 230G, 230Y, 231E, 231G, 231K, 231P, 231Y, 232E, 232G, 232K, 232Y, 233A, 233D, 233F, 233G, 233H, 233I, 233K, 233L, 233M, 233N, 233Q, 233R, 233S, 233T, 233V, 233W, 233Y, 234D, 234E, 234F, 234G, 234H, 234I, 234K, 234M, 234N, 234P, 234Q, 234R, 234S, 234T, 234W, 234Y, 235D, 235F, 235G, 235H, 235I, 235K, 235M, 235N, 235P, 235Q, 235R, 235S, 235T, 235V, 235W, 235Y, 236A, 236D, 236E, 236F, 236H, 236I, 236K, 236L, 236M, 236N, 236P, 236Q, 236R, 236S, 236T, 236V, 236W, 236Y, 237D, 237E, 237F, 237H, 237I, 237K, 237L, 237M, 237N, 237P, 237Q, 237R, 237S, 237T, 237V, 237W, 237Y, 238D, 238E, 238F, 238G, 238H, 238I, 238K, 238L, 238M, 238N, 238Q, 238R, 238S, 238T, 238V, 238W, 238Y, 239D, 239E, 239F, 239G, 239H, 239I, 239K, 239L, 239M, 239N, 239P, 239Q, 239R, 239T, 239V, 239W, 239Y, 240A, 240I, 240M, 240T, 241D, 241E, 241L, 241R, 241S, 241W, 241Y, 243E, 243H, 243L, 243Q, 243R, 243W, 243Y, 244H, 245A, 246D, 246E, 246H, 246Y, 247G, 247V, 249H, 249Q, 249Y, 255E, 255Y, 258H, 258S, 258Y, 260D, 260E, 260H, 260Y, 262A, 262E, 262F, 262I, 262T, 263A, 263I, 263M, 263T, 264A, 264D, 264E, 264F, 264G, 264H, 264I, 264K, 264L, 264M, 264N, 264P, 264Q, 264R, 264S, 264T, 264W, 264Y, 265F, 265G, 265H, 265I, 265K, 265L, 265M, 265P, 265Q, 265R, 265S, 265T, 265V, 265W, 265Y, 266A, 266I, 266M, 266T, 267D, 267E, 267F, 267H, 267I, 267K, 267L, 267M, 267N, 267P, 267Q, 267R, 267V, 267W, 267Y, 268D, 268E, 268F, 268G, 268I, 268K, 268L, 268M, 268P, 268R, 268T, 268V, 268W, 269F, 269G, 269H, 269I, 269K, 269L, 269M, 269N, 269P, 269R, 269S, 269T, 269V, 269W, 269Y, 270F, 270G, 270H, 270I, 270L, 270M, 270P, 270Q, 270R, 270S, 270T, 270W, 270Y, 271A, 271D, 271E, 271F, 271G, 271H, 271I, 271K, 271L, 271M, 271N, 271Q, 271R, 271S, 271T, 271V, 271W, 271Y, 272D, 272F, 272G, 272H, 272I, 272K, 272L, 272M, 272P, 272R, 272S, 272T, 272V, 272W, 272Y, 273I, 274D, 274E, 274F, 274G, 274H, 274I, 274L, 274M, 274N, 274P, 274R, 274T, 274V, 274W, 274Y, 275L, 275W, 276D, 276E, 276F, 276G, 276H, 276I, 276L, 276M, 276P, 276R, 276S, 276T, 276V, 276W, 276Y, 278D, 278E, 278G, 278H, 278I, 278K, 278L, 278M, 278N, 278P, 278Q, 278R, 278S, 278T, 278V, 278W, 280G, 280K, 280L, 280P, 280W, 281D, 281E, 281K, 281N, 281P, 281Q, 281Y, 282E, 282G, 282K, 282P, 282Y, 283G, 283H, 283K, 283L, 283P, 283R, 283Y, 284D, 284E, 284L, 284N, 284Q, 284T, 284Y, 285D, 285E, 285K, 285Q, 285W, 285Y, 286E, 286G, 286P, 286Y, 288D, 288E, 288Y, 290D, 290H, 290L, 290N, 290W, 291D, 291E, 291G, 291H, 291I, 291Q, 291T, 292D, 292E, 292T, 292Y, 293F, 293G, 293H, 293I, 293L, 293M, 293N, 293P, 293R, 293S, 293T, 293V, 293W, 293Y, 294F, 294G, 294H, 294I, 294K, 294L, 294M, 294P, 294R, 294S, 294T, 294V, 294W, 294Y, 295D, 295E, 295F, 295G, 295H, 295I, 295M, 295N, 295P, 295R, 295S, 295T, 295V, 295W, 295Y, 296A, 296D, 296E, 296G, 296I, 296K, 296L, 296M, 296N, 296Q, 296R, 296S, 296T, 296V, 297D, 297E, 297F, 297G, 297H, 297I, 297K, 297L, 297M, 297P, 297Q, 297R, 297S, 297T, 297V, 297W, 297Y, 298E, 298F, 298H, 298I, 298K, 298M, 298Q, 298R, 298W, 298Y, 299A, 299D, 299E, 299F, 299G, 299H, 299I, 299K, 299L, 299M, 299N, 299P, 299Q, 299R, 299S, 299V, 299W, 299Y, 300A, 300D, 300E, 300G, 300H, 300K, 300M, 300N, 300P, 300Q, 300R, 300S, 300T, 300V, 300W, 301D, 301E, 301H, 301Y, 302I, 303D, 303E, 303Y, 304D, 304H, 304L, 304N, 304T, 305E, 305T, 305Y, 313F, 317E, 317Q, 318H, 318L, 318Q, 318R, 318Y, 320D, 320F, 320G, 320H, 320I, 320L, 320N, 320P, 320S, 320T, 320V, 320W, 320Y, 322D, 322F, 322G, 322H, 322I, 322P, 322S, 322T, 322V, 322W, 322Y, 323I, 324D, 324F, 324G, 324H, 324I, 324L, 324M, 324P, 324R, 324T, 324V, 324W, 324Y, 325A, 325D, 325E, 325F, 325G, 325H, 325I, 325K, 325L, 325M, 325P, 325Q, 325R, 325S, 325T, 325V, 325W, 325Y, 326I, 326L, 326P, 326T, 327D, 327E, 327F, 327H, 327I, 327K, 327L, 327M, 327N, 327P, 327R, 327T, 327V, 327W, 327Y, 328A, 328D, 328E, 328F, 328G, 328H, 328I, 328K, 328M, 328N, 328P, 328Q, 328R, 328S, 328T, 328V, 328W, 328Y, 329D, 329E, 329F, 329G, 329H, 329I, 329K, 329L, 329M, 329N, 329Q, 329R, 329S, 329T, 329V, 329W, 329Y, 330E, 330F, 330G, 330H, 330I, 330L, 330M, 330N, 330P, 330R, 330T, 330V, 330W, 330Y, 331D, 331F, 331H, 331I, 331L, 331M, 331Q, 331R, 331T, 331V, 331W, 331Y, 332A, 332D, 332E, 332F, 332H, 332K, 332L, 332M, 332N, 332P, 332Q, 332R, 332S, 332T, 332V, 332W, 332Y, 333F, 333H, 333I, 333L, 333M, 333P, 333T, 333Y, 334F, 334I, 334P, 334T, 335D, 335F, 335G, 335H, 335I, 335L, 335M, 335N, 335P, 335R, 335S, 335V, 335W, 335Y, 336E, 336K, 336Y, 337E, 337H, and 337N.

In another aspect, a first modification selected from among

C131S, R133K, E137G, S138G, N192S, F193L, Q196K, T199I, D203N, T214K, T214R, R217P, R217L, R217S, C219S, C219T, C219Y, C220P, C220G, insertion of 221D, insertion of 221L, insertion of 221LGD, V222K, V222T, deletion of V222, insertion of 223T, E224H, E224P, insertion of 225T, insertion of 225P, P228R, substitution of P228 with RCPEPKSCDTPPPCPRCPEPKSCDTPPPCPRCPEPKSCDTPPPCPR, P228S, P233E, V234L, V234F, A235L, insertion of 236G, H268Q, Q274K, N276K, F296Y, F300Y, V309L, G327A, A330S, P331S, T339A, R355Q, E356D, M358L, N384S, K392N, M397V, K409R, Q419E, V422I, H435R, Y436F, and P445L. In a further variation, a second modification selected from among 221K, 221Y, 222E, 222Y, 223E, 223K, 224Y, 225E, 225K, 225W, 227E, 227G, 227K, 227Y, 228E, 228G, 228K, 228Y, 230A, 230E, 230G, 230Y, 231E, 231G, 231K, 231P, 231Y, 232E, 232G, 232K, 232Y, 233A, 233D, 233F, 233G, 233H, 233I, 233K, 233L, 233M, 233N, 233Q, 233R, 233S, 233T, 233V, 233W, 233Y, 234D, 234E, 234F, 234G, 234H, 234I, 234K, 234M, 234N, 234P, 234Q, 234R, 234S, 234T, 234W, 234Y, 235D, 235F, 235G, 235H, 235I, 235K, 235M, 235N, 235P, 235Q, 235R, 235S, 235T, 235V, 235W, 235Y, 236A, 236D, 236E, 236F, 236H, 236I, 236K, 236L, 236M, 236N, 236P, 236Q, 236R, 236S, 236T, 236V, 236W, 236Y, 237D, 237E, 237F, 237H, 237I, 237K, 237L, 237M, 237N, 237P, 237Q, 237R, 237S, 237T, 237V, 237W, 237Y, 238D, 238E, 238F, 238G, 238H, 238I, 238K, 238L, 238M, 238N, 238Q, 238R, 238S, 238T, 238V, 238W, 238Y, 239D, 239E, 239F, 239G, 239H, 239I, 239K, 239L, 239M, 239N, 239P, 239Q, 239R, 239T, 239V, 239W, 239Y, 240A, 240I, 240M, 240T, 241D, 241E, 241L, 241R, 241S, 241W, 241Y, 243E, 243H, 243L, 243Q, 243R, 243W, 243Y, 244H, 245A, 246D, 246E, 246H, 246Y, 247G, 247V, 249H, 249Q, 249Y, 255E, 255Y, 258H, 258S, 258Y, 260D, 260E, 260H, 260Y, 262A, 262E, 262F, 262I, 262T, 263A, 263I, 263M, 263T, 264A, 264D, 264E, 264F, 264G, 264H, 264I, 264K, 264L, 264M, 264N, 264P, 264Q, 264R, 264S, 264T, 264W, 264Y, 265F, 265G, 265H, 265I, 265K, 265L, 265M, 265P, 265Q, 265R, 265S, 265T, 265V, 265W, 265Y, 266A, 266I, 266M, 266T, 267D, 267E, 267F, 267H, 267I, 267K, 267L, 267M, 267N, 267P, 267Q, 267R, 267V, 267W, 267Y, 268D, 268E, 268F, 268G, 268I, 268K, 268L, 268M, 268P, 268R, 268T, 268V, 268W, 269F, 269G, 269H, 269I, 269K, 269L, 269M, 269N, 269P, 269R, 269S, 269T, 269V, 269W, 269Y, 270F, 270G, 270H, 270I, 270L, 270M, 270P, 270Q, 270R, 270S, 270T, 270W, 270Y, 271A, 271D, 271E, 271F, 271G, 271H, 271I, 271K, 271L, 271M, 271N, 271Q, 271R, 271S, 271T, 271V, 271W, 271Y, 272D, 272F, 272G, 272H, 272I, 272K, 272L, 272M, 272P, 272R, 272S, 272T, 272V, 272W, 272Y, 273I, 274D, 274E, 274F, 274G, 274H, 274I, 274L, 274M, 274N, 274P, 274R, 274T, 274V, 274W, 274Y, 275L, 275W, 276D, 276E, 276F, 276G, 276H, 276I, 276L, 276M, 276P, 276R, 276S, 276T, 276V, 276W, 276Y, 278D, 278E, 278G, 278H, 278I, 278K, 278L, 278M, 278N, 278P, 278Q, 278R, 278S, 278T, 278V, 278W, 280G, 280K, 280L, 280P, 280W, 281D, 281E, 281K, 281N, 281P, 281Q, 281Y, 282E, 282G, 282K, 282P, 282Y, 283G, 283H, 283K, 283L, 283P, 283R, 283Y, 284D, 284E, 284L, 284N, 284Q, 284T, 284Y, 285D, 285E, 285K, 285Q, 285W, 285Y, 286E, 286G, 286P, 286Y, 288D, 288E, 288Y, 290D, 290H, 290L, 290N, 290W, 291D, 291E, 291G, 291H, 291I, 291Q, 291T, 292D, 292E, 292T, 292Y, 293F, 293G, 293H, 293I, 293L, 293M, 293N, 293P, 293R, 293S, 293T, 293V, 293W, 293Y, 294F, 294G, 294H, 294I, 294K, 294L, 294M, 294P, 294R, 294S, 294T, 294V, 294W, 294Y, 295D, 295E, 295F, 295G, 295H, 295I, 295M, 295N, 295P, 295R, 295S, 295T, 295V, 295W, 295Y, 296A, 296D, 296E, 296G, 296I, 296K, 296L, 296M, 296N, 296Q, 296R, 296S, 296T, 296V, 297D, 297E, 297F, 297G, 297H, 297I, 297K, 297L, 297M, 297P, 297Q, 297R, 297S, 297T, 297V, 297W, 297Y, 298E, 298F, 298H, 298I, 298K, 298M, 298Q, 298R, 298W, 298Y, 299A, 299D, 299E, 299F, 299G, 299H, 299I, 299K, 299L, 299M, 299N, 299P, 299Q, 299R, 299S, 299V, 299W, 299Y, 300A, 300D, 300E, 300G, 300H, 300K, 300M, 300N, 300P, 300Q, 300R, 300S, 300T, 300V, 300W, 301D, 301E, 301H, 301Y, 302I, 303D, 303E, 303Y, 304D, 304H, 304L, 304N, 304T, 305E, 305T, 305Y, 313F, 317E, 317Q, 318H, 318L, 318Q, 318R, 318Y, 320D, 320F, 320G, 320H, 320I, 320L, 320N, 320P, 320S, 320T, 320V, 320W, 320Y, 322D, 322F, 322G, 322H, 322I, 322P, 322S, 322T, 322V, 322W, 322Y, 323I, 324D, 324F, 324G, 324H, 324I, 324L, 324M, 324P, 324R, 324T, 324V, 324W, 324Y, 325A, 325D, 325E, 325F, 325G, 325H, 325I, 325K, 325L, 325M, 325P, 325Q, 325R, 325S, 325T, 325V, 325W, 325Y, 326I, 326L, 326P, 326T, 327D, 327E, 327F, 327H, 327I, 327K, 327L, 327M, 327N, 327P, 327R, 327T, 327V, 327W, 327Y, 328A, 328D, 328E, 328F, 328G, 328H, 328I, 328K, 328M, 328N, 328P, 328Q, 328R, 328S, 328T, 328V, 328W, 328Y, 329D, 329E, 329F, 329G, 329H, 329I, 329K, 329L, 329M, 329N, 329Q, 329R, 329S, 329T, 329V, 329W, 329Y, 330E, 330F, 330G, 330H, 330I, 330L, 330M, 330N, 330P, 330R, 330T, 330V, 330W, 330Y, 331D, 331F, 331H, 331I, 331L, 331M, 331Q, 331R, 331T, 331V, 331W, 331Y, 332A, 332D, 332E, 332F, 332H, 332K, 332L, 332M, 332N, 332P, 332Q, 332R, 332S, 332T, 332V, 332W, 332Y, 333F, 333H, 333I, 333L, 333M, 333P, 333T, 333Y, 334F, 334I, 334P, 334T, 335D, 335F, 335G, 335H, 335I, 335L, 335M, 335N, 335P, 335R, 335S, 335V, 335W, 335Y, 336E, 336K, 336Y, 337E, 337H, 337N.

In another aspect, the present application is directed to an IgG2 variant including an amino acid sequence having the formula:

(SED ID NO: 90)

ASTKGPSVFPLAP-X(131)-S-X(133)-STS-X(137)-X(138)-TA
ALGCLVKDYFPEPVTVSWNSGALTSGVHTFPAVLQSSGLYSLSSVVTVPS
S-X(192)-X(193)-GT-X(196)-TY-X(199)-CNV-X(203)-HKP
SNTKVDK-X(214)-VE-X(217)-K-X(219)-X(220)-X(221)-
X(222)-X(223)-X(224)-X(225)-C-X(227)-X(228)-CPAP-
X(233)-X(234)-X(235)-X(236)-X(237)-P-X(239)-
X(240)-FLFPP-X(246)-PKDTLMIS-X(255)-TP-X(258)-V-
X(260)-CVV-X(264)-DV-X(267)-X(268)-ED-X(271)-
X(272)-V-X(274)-F-X(276)-W-X(278)-VD-X(281)-V-
X(283)-X(284)-HNAKT-X(290)-PR-X(293)-E-X(295)-
X(296)-NST-X(300)-RVV-X(304)-VLTV-X(309)-HQDWLNGKE
YKCKV-X(324)-N-X(326)-X(327)-X(328)-P-X(330)-
X(331)-X(332)-X(333)-X(334)-TISK-X(339)-KGQPREPQVY
TLPPS-X(355)-X(356)-E-X(358)-TKNQVSLTCLVKGFYPSDIAV
EWES-X(384)-GQPENNY-X(392)-TTPP-X(397)-
LDSDGSFFLYS-X(409)-LTVDKSRWQ-X(419)-GN-X(422)-FSCS
VMHEALHN-X(435)-X(436)-TQKSLSLS-X(445)-GK;

wherein
—X(131)- is selected from the group consisting of C and S;
—X(133)- is selected from the group consisting of R and K;
—X(137)- is selected from the group consisting of E and G;
—X(138)- is selected from the group consisting of S and G;
—X(192)- is selected from the group consisting of N and S;
—X(193)- is selected from the group consisting of F and L;
—X(196)- is selected from the group consisting of Q and K;
—X(199)- is selected from the group consisting of T and I;
—X(203)- is selected from the group consisting of D and N;
—X(214)- is selected from the group consisting of T, K and R;
—X(217)- is selected from the group consisting of R, P, L and S;
—X(219)- is selected from the group consisting of C, S, T and Y;
—X(220)- is selected from the group consisting of C, P and G;
—X(221)- is selected from the group consisting of no amino acid, D, K, L, and the sequence LGD;
—X(222)- is selected from the group consisting of V, K, T, and no amino acid;
—X(223)- is selected from the group consisting of no amino acid and T;
—X(224)- is selected from the group consisting of E, H and P;
—X(225)- is selected from the group consisting of no amino acid, T and P;
—X(227)- is selected from the group consisting of P and G;
—X(228)- is selected from the group consisting of P, S, R, and the sequence

(SEQ ID NO: 111)

RCPEPKSCDTPPPCPRCPEPKSCDTPPPCPRCPEPKSCDTPPPCPR;

—X(233)- is selected from the group consisting of P and E;
—X(234)- is selected from the group consisting of V, L, F, Y and I;
—X(235)- is selected from the group consisting of A, L, Y, I and D;
—X(236)- is selected from the group consisting of no amino acid, G, S and A;
—X(237)- is selected from the group consisting of G and D;
—X(239)- is selected from the group consisting of S, D, E, N, Q and T;
—X(240)- is selected from the group consisting of V, I and M;
—X(246)- is selected from the group consisting of K, H and Y;
—X(255)- is selected from the group consisting of R and Y;
—X(258)- is selected from the group consisting of E, H and Y;
—X(260)- is selected from the group consisting of T and H;
—X(264)- is selected from the group consisting of V, I, T and Y;
—X(267)- is selected from the group consisting of S, D and E;
—X(268)- is selected from the group consisting of H, Q, D and E;
—X(271)- is selected from the group consisting of P and G;
—X(272)- is selected from the group consisting of E, Y, H, R and I;
—X(274)- is selected from the group consisting of Q, K and E;
—X(276)- is selected from the group consisting of N and K;
—X(278)- is selected from the group consisting of Y and T;
—X(281)- is selected from the group consisting of G, D and E;
—X(283)- is selected from the group consisting of E, L and H;
—X(284)- is selected from the group consisting of V, E and D;
—X(290)- is selected from the group consisting of K and N;
—X(293)- is selected from the group consisting of E and R;
—X(295)- is selected from the group consisting of Q and E;
—X(296)- is selected from the group consisting of F and Y;
—X(300)- is selected from the group consisting of F and Y;
—X(304)- is selected from the group consisting of S and T;
—X(309)- is selected from the group consisting of V and L;
—X(324)- is selected from the group consisting of S, G and I;
—X(326)- is selected from the group consisting of K and T;
—X(327)- is selected from the group consisting of G, A and D;
—X(328)- is selected from the group consisting of L, A, F, I and T;
—X(330)- is selected from the group consisting of A, S, L, Y and I;
—X(331)- is selected from the group consisting of P and S;
—X(332)- is selected from the group consisting of I, D, E, N, Q and T;
—X(333)- is selected from the group consisting of E and Y;
—X(334)- is selected from the group consisting of K, F, I and T;
—X(339)- is selected from the group consisting of T and A;
—X(355)- is selected from the group consisting of R and Q;
—X(356)- is selected from the group consisting of E and D;
—X(358)- is selected from the group consisting of M and L;
—X(384)- is selected from the group consisting of N and S;
—X(392)- is selected from the group consisting of K and N;
—X(397)- is selected from the group consisting of M and V;
—X(409)- is selected from the group consisting of K and R;
—X(419)- is selected from the group consisting of Q and E;
—X(422)- is selected from the group consisting of V and I;
—X(435)- is selected from the group consisting of H and R;
—X(436)- is selected from the group consisting of Y and F; and
—X(445)- is selected from the group consisting of P and L.

In certain variations, a first modification is selected from among C131S, R133K, E137G, S138G, N192S, F193L, Q196K, T199I, D203N, T214K, T214R, R217P, R217L, R217S, C219S, C219T, C219Y, C220P, C220G, the insertion of 221D, the insertion of 221LGD, the insertion of 221L, V222K, V222T, the deletion of V222, the insertion of 223T, E224H, E224P, the insertion of 225T, the insertion of 225P, P228R, the substitution of RCPEPKSCDTPPPCPRCPEPKSCDTPPPCPRCPEPKSCDTPPPCPR (SEQ ID NO: 111) for P228, P228S, P233E, V234L, V234F, A235L, the insertion of 236G, H268Q, Q274K, N276K, F296Y, F300Y, V309L, G327A, A330S, P331S, T339A, R355Q, E356D, M358L, N384S, K392N, M397V, K409R, Q419E, V422I, H435R, Y436F, and P445L. In further variations, a second modification is selected from among 221K, 227G, 234Y, 234I, 235Y, 235I, 235D, 236S, 236A, 237D, 239D, 239E, 239N, 239Q, 239T, 240I, 240M, 246H, 246Y, 255Y, 258H, 258Y, 260H, 264I, 264T, 264Y, 267D, 267E, 268D, 268E, 271G, 272Y, 272H, 272R, 272I, 274E, 278T, 281D, 281E, 283L, 283H, 284E, 284D, 290N, 293R, 295E, 304T, 324G, 324I, 326T, 327D, 328A, 328F, 328I, 328T, 330L, 330Y, 330I, 332D, 332E, 332N, 332Q, 332T, 333Y, 334F, 334I, and 334T.

In another aspect, the present application is directed to an IgG2 variant including an amino acid sequence having the formula:

(SED ID NO: 91)

ASTKGPSVFPLAPCSRSTSESTAALGCLVKDYFPEPVTVSWNSGALTSGV
HTFPAVLQSSGLYSLSSVVTVPSSNFGTQTYTCNVDHKPSNTKVDKTVER
KCCVEC-X(227)-X(228)-CPAP-X(233)-X(234)-X(235)-
X(236)--X(237)-X(238)-X(239)-X(240)-X(241)-L-
X(243)-X(244)-X(245)-X(246)-X(247)-K-X(249)-TLMIS-
X(255)-TP-X(258)-V-X(260)-C-X(262)-X(263)-X(264)-
X(265)-X(266)-X(267)-X(268)-X(269)-X(270)-X(271)-
X(272)-X(273)-X(274)-X(275)-X(276)-W-X(278)-V-
X(280)-X(281)-X(282)-X(283)-X(284)-X(285)-X(286)-
A-X(288)-T-X(290)-X(291)-X(292)-X(293)-X(294)-
X(295)-X(296)-X(297)-X(298)-X(299)-X(300)-X(301)-
X(302)-X(303)-X(304)-X(305)-LTV-X(309)-HQD-X(313)-
LNG-X(317)-X(318)-Y-X(320)-C-X(322)-X(323)-X(324)-
X(325)-X(326)-X(327)-X(328)-X(329)-X(330)-X(331)-
X(332)-X(333)-X(334)-X(335)-X(336)-X(337)-K-
X(339)-KGQPREPQVYTLPPS-X(355)-X(356)-E-X(358)-TKNQ
VSLTCLVKGFYPSDIAVEWES-X(384)-GQPENNY-X(392)-TTPP-
X(397)-LDSDGSFFLYS-X(409)-LTVDKSRWQ-X(419)-GN-
X(422)-FSCSVMHEALHN-X(435)-X(436)-TQKSLSLS-X(445)-
GK,

wherein
—X(237)- is selected from the group consisting of G, D, E, F, H, I, K, L, M, N, P, Q, R, S, T, V, W and Y;
—X(238)- is selected from the group consisting of P, D, E, F, G, H, I, K, L, M, N, Q, R, S, T, V, W and Y;
—X(239)- is selected from the group consisting of S, D, E, F, G, H, I, K, L, M, N, P, Q, R, T, V, W and Y;
—X(240)- is selected from the group consisting of V, A, I, M and T;
—X(241)- is selected from the group consisting of F, D, E, L, R, S, W and Y;
—X(243)- is selected from the group consisting of F, E, H, L, Q, R, W, and Y;
—X(244)- is selected from the group consisting of P and H;
—X(245)- is selected from the group consisting of P and A;
—X(246)- is selected from the group consisting of, K, D, E, H and Y;
—X(247)- is selected from the group consisting of P, G and V;
—X(249)- is selected from the group consisting of D, H, Q and Y;
—X(255)- is selected from the group consisting of R, E and Y;
—X(258)- is selected from the group consisting of E, H, S and Y;
—X(260)- is selected from the group consisting of T, D, E, H and Y;
—X(262)- is selected from the group consisting of V, A, E, F, I and T;
—X(263)- is selected from the group consisting of V, A, I, M and T;
—X(264)- is selected from the group consisting of V, A, D, E, F, G, H, I, K, L, M, N, P, Q, R, S, T, W, and Y;
—X(265)- is selected from the group consisting of D, F, G, H, I, K, L, M, P, Q, R, S, T, V, W and Y;
—X(266)- is selected from the group consisting of V, A, I, M and T;
—X(267)- is selected from the group consisting of S, D, E, F, H, I, K, L, M, N, P, Q, R, V, W and Y;
—X(268)- is selected from the group consisting of H, Q, D, E, F, G, I, K, L, M, P, R, T, V and W;
—X(269)- is selected from the group consisting of E, F, G, H, I, K, L, M, N, P, R, S, T, V, W and Y;
—X(270)- is selected from the group consisting of D, F, G, H, I, L, M, P, Q, R, S, T, W and Y;
—X(271)- is selected from the group consisting of P, A, D, E, F, G, H, I, K, L, M, N, Q, R, S, T, V, W and Y;
—X(272)- is selected from the group consisting of E, D, F, G, H, I, K, L, M, P, R, S, T, V, W and Y;
—X(273)- is selected from the group consisting of V and I;
—X(274)- is selected from the group consisting of Q, K, D, E, F, G, H, I, L, M, N, P, R, T, V, W and Y;
—X(275)- is selected from the group consisting of F, L and W;
—X(276)- is selected from the group consisting of N, K, D, E, F, G, H, I, L, M, P, R, S, T, V, W and Y;
—X(278)- is selected from the group consisting of Y, D, E, G, H, I, K, L, M, N, P, Q, R, S, T, V and W;
—X(280)- is selected from the group consisting of D, G, K, L, P and W;
—X(281)- is selected from the group consisting of G, D, E, K, N, P, Q and Y;
—X(282)- is selected from the group consisting of V, E, G, K, P and Y;
—X(283)- is selected from the group consisting of E, G, H, K, L, P, R and Y;
—X(284)- is selected from the group consisting of V, D, E, L, N, Q, T and Y;
—X(285)- is selected from the group consisting of H, D, E, K, Q, W and Y;
—X(286)- is selected from the group consisting of N, E, G, P and Y;
—X(288)- is selected from the group consisting of K, D, E and Y;
—X(290)- is selected from the group consisting of K, D, H, L, N and W;
—X(291)- is selected from the group consisting of P, D, E, G, H, I, Q and T;
—X(292)- is selected from the group consisting of R, D, E, T and Y;
—X(293)- is selected from the group consisting of E, F, G, H, I, L, M, N, P, R, S, T, V, W and Y;
—X(294)- is selected from the group consisting of E, F, G, H, I, K, L, M, P, R, S, T, V, W and Y;
—X(295)- is selected from the group consisting of Q, D, E, F, G, H, I, M, N, P, R, S, T, V, W and Y;
—X(296)- is selected from the group consisting of F, Y, A, D, E, G, I, K, L, M, N, Q, R, S, T and V;
—X(297)- is selected from the group consisting of N, D, E, F, G, H, I, K, L, M, P, Q, R, S, T, V, W and Y;
—X(298)- is selected from the group consisting of S, E, F, H, I, K, M, Q, R, W and Y;
—X(299)- is selected from the group consisting of T, A, D, E, F, G, H, I, K, L, M, N, P, Q, R, S, V, W and Y;
—X(300)- is selected from the group consisting of F, Y, A, D, E, G, H, K, M, N, P, Q, R, S, T, V and W;
—X(301)- is selected from the group consisting of R, D, E, H and Y;
—X(302)- is selected from the group consisting of V and I;
—X(303)- is selected from the group consisting of V, D, E and Y;
—X(304)- is selected from the group consisting of S, D, H, L, N and T;
—X(305)- is selected from the group consisting of V, E, T and Y;
—X(309)- is selected from the group consisting of V and L;
—X(313)- is selected from the group consisting of W and F;
—X(317)- is selected from the group consisting of K, E and Q;
—X(318)- is selected from the group consisting of E, H, L, Q, R and Y;
—X(320)- is selected from the group consisting of K, D, F, G, H, I, L, N, P, S, T, V, W and Y;
—X(322)- is selected from the group consisting of K, D, F, G, H, I, P, S, T, V, W and Y;
—X(323)- is selected from the group consisting of V and I;
—X(324)- is selected from the group consisting of S, D, F, G, H, I, L, M, P, R, T, V, W and Y;
—X(325)- is selected from the group consisting of N, A, D, E, F, G, H, I, K, L, M, P, Q, R, S, T, V, W and Y;
—X(326)- is selected from the group consisting of K, I, L, P and T;
—X(327)- is selected from the group consisting of G, A, D, E, F, H, I, K, L, M, N, P, R, T, V, W and Y;
—X(328)- is selected from the group consisting of L, A, D, E, F, G, H, I, K, M, N, P, Q, R, S, T, V, W and Y;
—X(329)- is selected from the group consisting of P, D, E, F, G, H, I, K, L, M, N, Q, R, S, T, V, W and Y;
—X(330)- is selected from the group consisting of A, S, E, F, G, H, I, L, M, N, P, R, T, V, W and Y;
—X(331)- is selected from the group consisting of P, S, D, F, H, I, L, M, Q, R, T, V, W and Y;
—X(332)- is selected from the group consisting of I, A, D, E, F, H, K, L, M, N, P, Q, R, S, T, V, W and Y;
—X(333)- is selected from the group consisting of E, F, H, I, L, M, P, T and Y;
—X(334)- is selected from the group consisting of K, F, I, P and T;
—X(335)- is selected from the group consisting of T, D, F, G, H, I, L, M, N, P, R, S, V, W and Y;
—X(336)- is selected from the group consisting of I, E, K and Y;
—X(337)- is selected from the group consisting of S, E, H and N;
—X(339)- is selected from the group consisting of T and A;
—X(355)- is selected from the group consisting of R and Q;
—X(356)- is selected from the group consisting of E and D;
—X(358)- is selected from the group consisting of M and L;
—X(384)- is selected from the group consisting of N and S;
—X(392)- is selected from the group consisting of K and N;
—X(397)- is selected from the group consisting of M and V;
—X(409)- is selected from the group consisting of K and R;
—X(419)- is selected from the group consisting of Q and E;
—X(422)- is selected from the group consisting of V and I;
—X(435)- is selected from the group consisting of H and R;
—X(436)- is selected from the group consisting of Y and F; and
—X(445)- is selected from the group consisting of P and L.

In certain variations, a first modification is selected from among P228R, substitution of P228 with RCPEPKSCDTPPPCPRCPEPKSCDTPPPCPRCPEPKSCDTPPPCPR (SEQ ID NO: 111), P228S, P233E, V234L, V234F, A235L, insertion of 236G, H268Q, Q274K, N276K, F296Y, F300Y, V309L, G327A, A330S, P331S, T339A, R355Q, E356D, M358L, N384S, K392N, M397V, K409R, Q419E, V422I, H435R, Y436F, and P445L. In additional variations, a second modification is selected from among 227E, 227G, 227K, 227Y, 228E, 228G, 228K, 228Y, 230A, 230E, 230G, 230Y, 231E, 231G, 231K, 231P, 231Y, 232E, 232G, 232K, 232Y, 233A, 233D, 233F, 233G, 233H, 233I, 233K, 233L, 233M, 233N, 233Q, 233R, 233S, 233T, 233V, 233W, 233Y, 234D, 234E, 234F, 234G, 234H, 234I, 234K, 234M, 234N, 234P, 234Q, 234R, 234S, 234T, 234W, 234Y, 235D, 235F, 235G, 235H, 235I, 235K, 235M, 235N, 235P, 235Q, 235R, 235S, 235T, 235V, 235W, 235Y, 236A, 236D, 236E, 236F, 236H, 236I, 236K, 236L, 236M, 236N, 236P, 236Q, 236R, 236S, 236T, 236V, 236W, 236Y, 237D, 237E, 237F, 237H, 237I, 237K, 237L, 237M, 237N, 237P, 237Q, 237R, 237S, 237T, 237V, 237W, 237Y, 238D, 238E, 238F, 238G, 238H, 238I, 238K, 238L, 238M, 238N, 238Q, 238R, 238S, 238T, 238V, 238W, 238Y, 239D, 239E, 239F, 239G, 239H, 239I, 239K, 239L, 239M, 239N, 239P, 239Q, 239R, 239T, 239V, 239W, 239Y, 240A, 240I, 240M, 240T, 241D, 241E, 241L, 241R, 241S, 241W, 241Y, 243E, 243H, 243L, 243Q, 243R, 243W, 243Y, 244H, 245A, 246D, 246E, 246H, 246Y, 247G, 247V, 249H, 249Q, 249Y, 255E, 255Y, 258H, 258S, 258Y, 260D, 260E, 260H, 260Y, 262A, 262E, 262F, 262I, 262T, 263A, 263I, 263M, 263T, 264A, 264D, 264E, 264F, 264G, 264H, 264I, 264K, 264L, 264M, 264N, 264P, 264Q, 264R, 264S, 264T, 264W, 264Y, 265F, 265G, 265H, 265I, 265K, 265L, 265M, 265P, 265Q, 265R, 265S, 265T, 265V, 265W, 265Y, 266A, 266I, 266M, 266T, 267D, 267E, 267F, 267H, 267I, 267K, 267L, 267M, 267N, 267P, 267Q, 267R, 267V, 267W, 267Y, 268D, 268E, 268F, 268G, 268I, 268K, 268L, 268M, 268P, 268R, 268T, 268V, 268W, 269F, 269G, 269H, 269I, 269K, 269L, 269M, 269N, 269P, 269R, 269S, 269T, 269V, 269W, 269Y, 270F, 270G, 270H, 270I, 270L, 270M, 270P, 270Q, 270R, 270S, 270T, 270W, 270Y, 271A, 271D, 271E, 271F, 271G, 271H, 271I, 271K, 271L, 271M, 271N, 271Q, 271R, 271S, 271T, 271V, 271W, 271Y, 272D, 272F, 272G, 272H, 272I, 272K, 272L, 272M, 272P, 272R, 272S, 272T, 272V, 272W, 272Y, 273I, 274D, 274E, 274F, 274G, 274H, 274I, 274L, 274M, 274N, 274P, 274R, 274T, 274V, 274W, 274Y, 275L, 275W, 276D, 276E, 276F, 276G, 276H, 276I, 276L, 276M, 276P, 276R, 276S, 276T, 276V, 276W, 276Y, 278D, 278E, 278G, 278H, 278I, 278K, 278L, 278M, 278N, 278P, 278Q, 278R, 278S, 278T, 278V, 278W, 280G, 280K, 280L, 280P, 280W, 281D, 281E, 281K, 281N, 281P, 281Q, 281Y, 282E, 282G, 282K, 282P, 282Y, 283G, 283H, 283K, 283L, 283P, 283R, 283Y, 284D, 284E, 284L, 284N, 284Q, 284T, 284Y, 285D, 285E, 285K, 285Q, 285W, 285Y, 286E, 286G, 286P, 286Y, 288D, 288E, 288Y, 290D, 290H, 290L, 290N, 290W, 291D, 291E, 291G, 291H, 291I, 291Q, 291T, 292D, 292E, 292T, 292Y, 293F, 293G, 293H, 293I, 293L, 293M, 293N, 293P, 293R, 293S, 293T, 293V, 293W, 293Y, 294F, 294G, 294H, 294I, 294K, 294L, 294M, 294P, 294R, 294S, 294T, 294V, 294W, 294Y, 295D, 295E, 295F, 295G, 295H, 295I, 295M, 295N, 295P, 295R, 295S, 295T, 295V, 295W, 295Y, 296A, 296D, 296E, 296G, 296I, 296K, 296L, 296M, 296N, 296Q, 296R, 296S, 296T, 296V, 297D, 297E, 297F, 297G, 297H, 297I, 297K, 297L, 297M, 297P, 297Q, 297R, 297S, 297T, 297V, 297W, 297Y, 298E, 298F, 298H, 298I, 298K, 298M, 298Q, 298R, 298W, 298Y, 299A, 299D, 299E, 299F, 299G, 299H, 299I, 299K, 299L, 299M, 299N, 299P, 299Q, 299R, 299S, 299V, 299W, 299Y, 300A, 300D, 300E, 300G, 300H, 300K, 300M, 300N, 300P, 300Q, 300R, 300S, 300T, 300V, 300W, 301D, 301E, 301H, 301Y, 302I, 303D, 303E, 303Y, 304D, 304H, 304L, 304N, 304T, 305E, 305T, 305Y, 313F, 317E, 317Q, 318H, 318L, 318Q, 318R, 318Y, 320D, 320F, 320G, 320H, 320I, 320L, 320N, 320P, 320S, 320T, 320V, 320W, 320Y, 322D, 322F, 322G, 322H, 322I, 322P, 322S, 322T, 322V, 322W, 322Y, 323I, 324D, 324F, 324G, 324H, 324I, 324L, 324M, 324P, 324R, 324T, 324V, 324W, 324Y, 325A, 325D, 325E, 325F, 325G, 325H, 325I, 325K, 325L, 325M, 325P, 325Q, 325R, 325S, 325T, 325V, 325W, 325Y, 326I, 326L, 326P, 326T, 327D, 327E, 327F, 327H, 327I, 327K, 327L, 327M, 327N, 327P, 327R, 327T, 327V, 327W, 327Y, 328A, 328D, 328E, 328F, 328G, 328H, 328I, 328K, 328M, 328N, 328P, 328Q, 328R, 328S, 328T, 328V, 328W, 328Y, 329D, 329E, 329F, 329G, 329H, 329I, 329K, 329L, 329M, 329N, 329Q, 329R, 329S, 329T, 329V, 329W, 329Y, 330E, 330F, 330G, 330H, 330I, 330L, 330M, 330N, 330P, 330R, 330T, 330V, 330W, 330Y, 331D, 331F, 331H, 331I, 331L, 331M, 331Q, 331R, 331T, 331V, 331W, 331Y, 332A, 332D, 332E, 332F, 332H, 332K, 332L, 332M, 332N, 332P, 332Q, 332R, 332S, 332T, 332V, 332W, 332Y, 333F, 333H, 333I, 333L, 333M, 333P, 333T, 333Y, 334F, 334I, 334P, 334T, 335D, 335F, 335G, 335H, 335I, 335L, 335M, 335N, 335P, 335R, 335S, 335V, 335W, 335Y, 336E, 336K, 336Y, 337E, 337H, and 337N. In certain variations, X(227) is P and X(228) is P.

In a further aspect, the present application is directed to an IgG2 variant amino acid sequence including at least two modifications as compared to SEQ ID NO:2. In certain variations, a first modification is selected from among P228R, substitution of P228 with RCPEPKSCDTPPPCPRCPEPKSCDTPPPCPRCPEPKSCDTPPPCPR (SEQ ID NO: 111), P228S, P233E, V234L, V234F, A235L, insertion of 236G, H268Q, Q274K, N276K, F296Y, F300Y, V309L, G327A, A330S, P331S, T339A, R355Q, E356D, M358L, N384S, K392N, M397V, K409R, Q419E, V422I, H435R, Y436F, and P445L. In further variations, a second modification is selected from among 227E, 227G, 227K, 227Y, 228E, 228G, 228K, 228Y, 230A, 230E, 230G, 230Y, 231E, 231G, 231K, 231P, 231Y, 232E, 232G, 232K, 232Y, 233A, 233D, 233F, 233G, 233H, 233I, 233K, 233L, 233M, 233N, 233Q, 233R, 233S, 233T, 233V, 233W, 233Y, 234D, 234E, 234F, 234G, 234H, 234I, 234K, 234M, 234N, 234P, 234Q, 234R, 234S, 234T, 234W, 234Y, 235D, 235F, 235G, 235H, 235I, 235K, 235M, 235N, 235P, 235Q, 235R, 235S, 235T, 235V, 235W, 235Y, 236A, 236D, 236E, 236F, 236H, 236I, 236K, 236L, 236M, 236N, 236P, 236Q, 236R, 236S, 236T, 236V, 236W, 236Y, 237D, 237E, 237F, 237H, 237I, 237K, 237L, 237M, 237N, 237P, 237Q, 237R, 237S, 237T, 237V, 237W, 237Y, 238D, 238E, 238F, 238G, 238H, 238I, 238K, 238L, 238M, 238N, 238Q, 238R, 238S, 238T, 238V, 238W, 238Y, 239D, 239E, 239F, 239G, 239H, 239I, 239K, 239L, 239M, 239N, 239P, 239Q, 239R, 239T, 239V, 239W, 239Y, 240A, 240I, 240M, 240T, 241D, 241E, 241L, 241R, 241S, 241W, 241Y, 243E, 243H, 243L, 243Q, 243R, 243W, 243Y, 244H, 245A, 246D, 246E, 246H, 246Y, 247G, 247V, 249H, 249Q, 249Y, 255E, 255Y, 258H, 258S, 258Y, 260D, 260E, 260H, 260Y, 262A, 262E, 262F, 262I, 262T, 263A, 263I, 263M, 263T, 264A, 264D, 264E, 264F, 264G, 264H, 264I, 264K, 264L, 264M, 264N, 264P, 264Q, 264R, 264S, 264T, 264W, 264Y, 265F, 265G, 265H, 265I, 265K, 265L, 265M, 265P, 265Q, 265R, 265S, 265T, 265V, 265W, 265Y, 266A, 266I, 266M, 266T, 267D, 267E, 267F, 267H, 267I, 267K, 267L, 267M, 267N, 267P, 267Q, 267R, 267V, 267W, 267Y, 268D, 268E, 268F, 268G, 268I, 268K, 268L, 268M, 268P, 268R, 268T, 268V, 268W, 269F, 269G, 269H, 269I, 269K, 269L, 269M, 269N, 269P, 269R, 269S, 269T, 269V, 269W, 269Y, 270F, 270G, 270H, 270I, 270L, 270M, 270P, 270Q, 270R, 270S, 270T, 270W, 270Y, 271A, 271D, 271E, 271F, 271G, 271H, 271I, 271K, 271L, 271M, 271N, 271Q, 271R, 271S, 271T, 271V, 271W, 271Y, 272D, 272F, 272G, 272H, 272I, 272K, 272L, 272M, 272P, 272R, 272S, 272T, 272V, 272W, 272Y, 273I, 274D, 274E, 274F, 274G, 274H, 274I, 274L, 274M, 274N, 274P, 274R, 274T, 274V, 274W, 274Y, 275L, 275W, 276D, 276E, 276F, 276G, 276H, 276I, 276L, 276M, 276P, 276R, 276S, 276T, 276V, 276W, 276Y, 278D, 278E, 278G, 278H, 278I, 278K, 278L, 278M, 278N, 278P, 278Q, 278R, 278S, 278T, 278V, 278W, 280G, 280K, 280L, 280P, 280W, 281D, 281E, 281K, 281N, 281P, 281Q, 281Y, 282E, 282G, 282K, 282P, 282Y, 283G, 283H, 283K, 283L, 283P, 283R, 283Y, 284D, 284E, 284L, 284N, 284Q, 284T, 284Y, 285D, 285E, 285K, 285Q, 285W, 285Y, 286E, 286G, 286P, 286Y, 288D, 288E, 288Y, 290D, 290H, 290L, 290N, 290W, 291D, 291E, 291G, 291H, 291I, 291Q, 291T, 292D, 292E, 292T, 292Y, 293F, 293G, 293H, 293I, 293L, 293M, 293N, 293P, 293R, 293S, 293T, 293V, 293W, 293Y, 294F, 294G, 294H, 294I, 294K, 294L, 294M, 294P, 294R, 294S, 294T, 294V, 294W, 294Y, 295D, 295E, 295F, 295G, 295H, 295I, 295M, 295N, 295P, 295R, 295S, 295T, 295V, 295W, 295Y, 296A, 296D, 296E, 296G, 296I, 296K, 296L, 296M, 296N, 296Q, 296R, 296S, 296T, 296V, 297D, 297E, 297F, 297G, 297H, 297I, 297K, 297L, 297M, 297P, 297Q, 297R, 297S, 297T, 297V, 297W, 297Y, 298E, 298F, 298H, 298I, 298K, 298M, 298Q, 298R, 298W, 298Y, 299A, 299D, 299E, 299F, 299G, 299H, 299I, 299K, 299L, 299M, 299N, 299P, 299Q, 299R, 299S, 299V, 299W, 299Y, 300A, 300D, 300E, 300G, 300H, 300K, 300M, 300N, 300P, 300Q, 300R, 300S, 300T, 300V, 300W, 301D, 301E, 301H, 301Y, 302I, 303D, 303E, 303Y, 304D, 304H, 304L, 304N, 304T, 305E, 305T, 305Y, 313F, 317E, 317Q, 318H, 318L, 318Q, 318R, 318Y, 320D, 320F, 320G, 320H, 320I, 320L, 320N, 320P, 320S, 320T, 320V, 320W, 320Y, 322D, 322F, 322G, 322H, 322I, 322P, 322S, 322T, 322V, 322W, 322Y, 323I, 324D, 324F, 324G, 324H, 324I, 324L, 324M, 324P, 324R, 324T, 324V, 324W, 324Y, 325A, 325D, 325E, 325F, 325G, 325H, 325I, 325K, 325L, 325M, 325P, 325Q, 325R, 325S, 325T, 325V, 325W, 325Y, 326I, 326L, 326P, 326T, 327D, 327E, 327F, 327H, 327I, 327K, 327L, 327M, 327N, 327P, 327R, 327T, 327V, 327W, 327Y, 328A, 328D, 328E, 328F, 328G, 328H, 328I, 328K, 328M, 328N, 328P, 328Q, 328R, 328S, 328T, 328V, 328W, 328Y, 329D, 329E, 329F, 329G, 329H, 329I, 329K, 329L, 329M, 329N, 329Q, 329R, 329S, 329T, 329V, 329W, 329Y, 330E, 330F, 330G, 330H, 330I, 330L, 330M, 330N, 330P, 330R, 330T, 330V, 330W, 330Y, 331D, 331F, 331H, 331I, 331L, 331M, 331Q, 331R, 331T, 331V, 331W, 331Y, 332A, 332D, 332E, 332F, 332H, 332K, 332L, 332M, 332N, 332P, 332Q, 332R, 332S, 332T, 332V, 332W, 332Y, 333F, 333H, 333I, 333L, 333M, 333P, 333T, 333Y, 334F, 334I, 334P, 334T, 335D, 335F, 335G, 335H, 335I, 335L, 335M, 335N, 335P, 335R, 335S, 335V, 335W, 335Y, 336E, 336K, 336Y, 337E, 337H, and 337N.

In a further aspect, the application is directed to an IgG2 variant including an amino acid sequence having the formula:

(SED ID NO: 92)

ASTKGPSVFPLAPCSRSTSESTAALGCLVKDYFPEPVTVSWNSGALTSGV
HTFPAVLQSSGLYSLSSVVTVPSSNFGTQTYTCNVDHKPSNTKVDKTVER
KCCVEC-X(227)-X(228)-CPAP-X(233)-X(234)-X(235)-
X(236)-X(237)-P-X(239)-X(240)-FLFPP-X(246)-PKDTLMI
S-X(255)-TP-X(258)-V-X(260)-CVV-X(264)-DV-X(267)-
X(268)-ED-X(271)-X(272)-V-X(274)-F-X(276)-W-
X(278)-VD-X(281)-V-X(283)-X(284)-HNAKT-X(290)-PR-
X(293)-E-X(295)-X(296)-NST-X(300)-RVV-X(304)-VLTV-
X(309)-HQDWLNGKEYKCKV-X(324)-N-X(326)-X(327)-
X(328)-P-X(330)-X(331)-X(332)-X(333)-X(334)-TISK-
X(339)-KGQPREPQVYTLPPS-X(355)-X(356)-E-X(358)-TKNQ
VSLTCLVKGFYPSDIAVEWES-X(384)-GQPENNY-X(392)-TTPP-
X(397)-LDSDGSFFLYS-X(409)-LTVDKSRWQ-X(419)-GN-
X(422)-FSCSVMHEALHN-X(435)-X(436)-TQKSLSLS-X(445)-
GK,

wherein
—X(237)- is selected from the group consisting of G and D;
—X(239)- is selected from the group consisting of S, D, E, N, Q and T;
—X(240)- is selected from the group consisting of V, I and M;
—X(246)- is selected from the group consisting of K, H and Y;
—X(255)- is selected from the group consisting of R and Y;
—X(258)- is selected from the group consisting of E, H and Y;
—X(260)- is selected from the group consisting of T and H;
—X(264)- is selected from the group consisting of V, I, T and Y;
—X(267)- is selected from the group consisting of S, D and E;
—X(268)- is selected from the group consisting of H, Q, D and E;
—X(271)- is selected from the group consisting of P and G;
—X(272)- is selected from the group consisting of E, Y, H, R and I;
—X(274)- is selected from the group consisting of Q, K and E;
—X(276)- is selected from the group consisting of N and K;
—X(278)- is selected from the group consisting of Y and T;
—X(281)- is selected from the group consisting of G, D and E;
—X(283)- is selected from the group consisting of E, L and H;
—X(284)- is selected from the group consisting of V, E and D;
—X(290)- is selected from the group consisting of K and N;
—X(293)- is selected from the group consisting of E and R;
—X(295)- is selected from the group consisting of Q and E;
—X(296)- is selected from the group consisting of F and Y;
—X(300)- is selected from the group consisting of F and Y;
—X(304)- is selected from the group consisting of S and T;
—X(309)- is selected from the group consisting of V and L;
—X(324)- is selected from the group consisting of S, G and I;
—X(326)- is selected from the group consisting of K and T;
—X(327)- is selected from the group consisting of G, A and D;
—X(328)- is selected from the group consisting of L, A, F, I and T;
—X(330)- is selected from the group consisting of A, S, L, Y and I;
—X(331)- is selected from the group consisting of P and S;
—X(332)- is selected from the group consisting of I, D, E, N, Q and T;
—X(333)- is selected from the group consisting of E and Y;
—X(334)- is selected from the group consisting of K, F, I and T;
—X(339)- is selected from the group consisting of T and A;
—X(355)- is selected from the group consisting of R and Q;
—X(356)- is selected from the group consisting of E and D;
—X(358)- is selected from the group consisting of M and L;
—X(384)- is selected from the group consisting of N and S;
—X(392)- is selected from the group consisting of K and N;
—X(397)- is selected from the group consisting of M and V;
—X(409)- is selected from the group consisting of K and R;
—X(419)- is selected from the group consisting of Q and E;
—X(422)- is selected from the group consisting of V and I;
—X(435)- is selected from the group consisting of H and R;
—X(436)- is selected from the group consisting of Y and F;
—X(445)- is selected from the group consisting of P and L;

In certain variations, a first modification is selected from among P228R, the substitution of RCPEPKSCDTPPPCPRCPEPKSCDTPPPCPRCPEPKSCDTPPPCPR (SEQ ID NO: 111) for P228, P228S, P233E, V234L, V234F, A235L, the insertion of 236G, H268Q, K274Q, N276K, Y296F, Y300F, L309V, A327G, A330S, P331S, A339T, R355Q, D356E, L358M, N384S, K392N, V397M, K409R, Q419E, V422I, H435R, Y436F, and P445L. In additional variations, a second modification is selected from among 227G, 234Y, 234I, 235Y, 235I, 235D, 236S, 236A, 237D, 239D, 239E, 239N, 239Q, 239T, 240I, 240M, 246H, 246Y, 255Y, 258H, 258Y, 260H, 264I, 264T, 264Y, 267D, 267E, 268D, 268E, 271G, 272Y, 272H, 272R, 272I, 274E, 278T, 281D, 281E, 283L, 283H, 284E, 284D, 290N, 293R, 295E, 304T, 324G, 324I, 326T, 327D, 328A, 328F, 328I, 328T, 330L, 330Y, 330I, 332D, 332E, 332N, 332Q, 332T, 333Y, 334F, 334I, and 334T.

In another aspect, the present application is directed to an IgG2 variant including an amino acid sequence having the formula:

(SED ID NO: 93)

ASTKGPSVFPLAPCSRSTSESTAALGCLVKDYFPEPVTVSWNSGALTSGV
HTFPAVLQSSGLYSLSSVVTVPSSNFGTQTYTCNVDHKPSNTKVDKTVER
KCC-X(221)-X(222)-X(223)-X(224)-X(225)-C-X(227)-
X(228)-C-X(230)-X(231)-X(232)-ELLGG-X(238)-X(239)-
X(240)-X(241)-L-X(243)-X(244)-X(245)-X(246)-
X(247)-K-X(249)-TLMIS-X(255)-TP-X(258)-V-X(260)-C-
X(262)-X(263)-X(264)-X(265)-X(266)-X(267)-X(268)-
X(269)-X(270)-X(271)-X(272)-X(273)-X(274)-X(275)-
X(276)-W-X(278)-V-X(280)-X(281)-X(282)-X(283)-
X(284)-X(285)-X(286)-A-X(288)-T-X(290)-X(291)-
X(292)-X(293)-X(294)-X(295)-X(296)-X(297)-X(298)-
X(299)-X(300)-X(301)-X(302)-X(303)-X(304)-X(305)-
LTVVHQD-X(313)-LNG-X(317)-X(318)-Y-X(320)-C-
X(322)-X(323)-X(324)-X(325)-X(326)-X(327)-X(328)-
X(329)-X(330)-X(331)-X(332)-X(333)-X(334)-X(335)-
X(336)-X(337)-KTKGQPREPQVYTLPPSREEMTKNQVSLTCLVKGFY
PSDIAVEWESNGQPENNYKTTPPMLDSDGSFFLYSKLTVDKSRWQQGNVF
SCSVMHEALHNHYTQKSLSLSPGK-,

wherein
—X(221)- is selected from the group consisting of no amino acid, K and Y;
—X(222)- is selected from the group consisting of V, E and Y;
—X(223)- is selected from the group consisting of no amino acid, E and K;
—X(224)- is selected from the group consisting of E and Y;
—X(225)- is selected from the group consisting of no amino acid, E, K and W;
—X(227)- is selected from the group consisting of P, E, G, K and Y;
—X(228)- is selected from the group consisting of P, E, G, K and Y;
—X(230)- is selected from the group consisting of P, A, E, G and Y;
—X(231)- is selected from the group consisting of A, E, G, K, P and Y;
—X(232)- is selected from the group consisting of P, E, G, K and Y;
—X(233)- is selected from the group consisting of P, A, D, F, G, H, I, K, L, M, N, Q, R, S, T, V, W and Y;
—X(234)- is selected from the group consisting of V, D, E, F, G, H, I, K, M, N, P, Q, R, S, T, W and Y;
—X(235)- is selected from the group consisting of A, D, F, G, H, I, K, M, N, P, Q, R, S, T, V, W and Y;
—X(236)- is selected from the group consisting of no amino acid, A, D, E, F, H, I, K, L, M, N, P, Q, R, S, T, V, W and Y;
—X(237)- is selected from the group consisting of G, D, E, F, H, I, K, L, M, N, P, Q, R, S, T, V, W and Y;
—X(238)- is selected from the group consisting of P, D, E, F, G, H, I, K, L, M, N, Q, R, S, T, V, W and Y;
—X(239)- is selected from the group consisting of S, D, E, F, G, H, I, K, L, M, N, P, Q, R, T, V, W and Y;
—X(240)- is selected from the group consisting of V, A, I, M and T;
—X(241)- is selected from the group consisting of F, D, E, L, R, S, W and Y;
—X(243)- is selected from the group consisting of F, E, H, L, Q, R, W and Y;
—X(244)- is selected from the group consisting of P and H;
—X(245)- is selected from the group consisting of P and A;
—X(246)- is selected from the group consisting of K, D, E, H and Y;
—X(247)- is selected from the group consisting of P, G and V;
—X(249)- is selected from the group consisting of D, H, Q and Y;
—X(255)- is selected from the group consisting of R, E and Y;
—X(258)- is selected from the group consisting of E, H, S and Y;
—X(260)- is selected from the group consisting of T, D, E, H and Y;
—X(262)- is selected from the group consisting of V, A, E, F, I and T;
—X(263)- is selected from the group consisting of V, A, I, M and T;
—X(264)- is selected from the group consisting of V, A, D, E, F, G, H, I, K, L, M, N, P, Q, R, S, T, W and Y;
—X(265)- is selected from the group consisting of D, F, G, H, I, K, L, M, P, Q, R, S, T, V, W and Y;
—X(266)- is selected from the group consisting of V, A, I, M and T;
—X(267)- is selected from the group consisting of S, D, E, F, H, I, K, L, M, N, P, Q, R, V, W and Y;
—X(268)- is selected from the group consisting of H, D, E, F, G, I, K, L, M, P, R, T, V and W;
—X(269)- is selected from the group consisting of E, F, G, H, I, K, L, M, N, P, R, S, T, V, W and Y;
—X(270)- is selected from the group consisting of D, F, G, H, I, L, M, P, Q, R, S, T, W and Y;
—X(271)- is selected from the group consisting of P, A, D, E, F, G, H, I, K, L, M, N, Q, R, S, T, V, W and Y;
—X(272)- is selected from the group consisting of E, D, F, G, H, I, K, L, M, P, R, S, T, V, W and Y;
—X(273)- is selected from the group consisting of V and I;
—X(274)- is selected from the group consisting of Q, D, E, F, G, H, I, L, M, N, P, R, T, V, W and Y;
—X(275)- is selected from the group consisting of F, L and W;
—X(276)- is selected from the group consisting of N, D, E, F, G, H, I, L, M, P, R, S, T, V, W and Y;
—X(278)- is selected from the group consisting of Y, D, E, G, H, I, K, L, M, N, P, Q, R, S, T, V and W;
—X(280)- is selected from the group consisting of D, G, K, L, P and W;
—X(281)- is selected from the group consisting of G, D, E, K, N, P, Q and Y;
—X(282)- is selected from the group consisting of V, E, G, K, P and Y;
—X(283)- is selected from the group consisting of E, G, H, K, L, P, R and Y;
—X(284)- is selected from the group consisting of V, D, E, L, N, Q, T and Y;
—X(285)- is selected from the group consisting of H, D, E, K, Q, W and Y;
—X(286)- is selected from the group consisting of N, E, G, P and Y;
—X(288)- is selected from the group consisting of K, D, E and Y;
—X(290)- is selected from the group consisting of K, D, H, L, N and W;
—X(291)- is selected from the group consisting of P, D, E, G, H, I, Q and T;
—X(292)- is selected from the group consisting of R, D, E, T and Y;
—X(293)- is selected from the group consisting of E, F, G, H, I, L, M, N, P, R, S, T, V, W and Y;
—X(294)- is selected from the group consisting of E, F, G, H, I, K, L, M, P, R, S, T, V, W and Y;
—X(295)- is selected from the group consisting of Q, D, E, F, G, H, I, M, N, P, R, S, T, V, W and Y;
—X(296)- is selected from the group consisting of F, A, D, E, G, I, K, L, M, N, Q, R, S, T and V;
—X(297)- is selected from the group consisting of N, D, E, F, G, H, I, K, L, M, P, Q, R, S, T, V, W and Y;
—X(298)- is selected from the group consisting of S, E, F, H, I, K, M, Q, R, W and Y;
—X(299)- is selected from the group consisting of T, A, D, E, F, G, H, I, K, L, M, N, P, Q, R, S, V, W and Y;
—X(300)- is selected from the group consisting of F, A, D, E, G, H, K, M, N, P, Q, R, S, T, V and W;
—X(301)- is selected from the group consisting of R, D, E, H and Y;
—X(302)- is selected from the group consisting of V and I;
—X(303)- is selected from the group consisting of V, D, E and Y;
—X(304)- is selected from the group consisting of S, D, H, L, N and T;
—X(305)- is selected from the group consisting of V, E, T and Y;
—X(313)- is selected from the group consisting of W and F;
—X(317)- is selected from the group consisting of K, E and Q;
—X(318)- is selected from the group consisting of E, H, L, Q, R and Y;
—X(320)- is selected from the group consisting of K, D, F, G, H, I, L, N, P, S, T, V, W and Y;
—X(322)- is selected from the group consisting of K, D, F, G, H, I, P, S, T, V, W and Y;
—X(323)- is selected from the group consisting of V and I;
—X(324)- is selected from the group consisting of S, D, F, G, H, I, L, M, P, R, T, V, W and Y;
—X(325)- is selected from the group consisting of N, A, D, E, F, G, H, I, K, L, M, P, Q, R, S, T, V, W and Y;
—X(326)- is selected from the group consisting of K, I, L, P and T;
—X(327)- is selected from the group consisting of A, G, D, E, F, H, I, K, L, M, N, P, R, T, V, W and Y;
—X(328)- is selected from the group consisting of L, A, D, E, F, G, H, I, K, M, N, P, Q, R, S, T, V, W and Y;
—X(329)- is selected from the group consisting of P, D, E, F, G, H, I, K, L, M, N, Q, R, S, T, V, W and Y;
—X(330)- is selected from the group consisting of A, E, F, G, H, I, L, M, N, P, R, T, V, W and Y;
—X(331)- is selected from the group consisting of P, D, F, H, I, L, M, Q, R, T, V, W and Y;
—X(332)- is selected from the group consisting of I, A, D, E, F, H, K, L, M, N, P, Q, R, S, T, V, W and Y;
—X(333)- is selected from the group consisting of E, F, H, I, L, M, P, T and Y;
—X(334)- is selected from the group consisting of K, F, I, P and T;
—X(335)- is selected from the group consisting of T, D, F, G, H, I, L, M, N, P, R, S, V, W and Y;
—X(336)- is selected from the group consisting of I, E, K and Y; and
—X(337)- is selected from the group consisting of S, E, H and N.

The variant differs from SEQ ID NO:2 by at least one amino acid. In a further aspect, X(327) is A.

In another aspect, the present application is directed to an IgG2 variant including an amino acid sequence having the formula:

(SED ID NO: 94)

ASTKGPSVFPLAPCSRSTSESTAALGCLVKDYFPEPVTVSWNSGALTSGV
HTFPAVLQSSGLYSLSSVVTVPSSNFGTQTYTCNVDHKPSNTKVDKTVER
KCC-X(221)-VEC-X(227)-PCPAPELLGGP-X(239)-X(240)-FL
FPP-X(246)-PKDTLMIS-X(255)-TP-X(258)-V-X(260)-CVV-
X(264)-DV-X(267)-X(268)-ED-X(271)-X(272)-V-X(274)-
FNW-X(278)-VD-X(281)-V-X(283)-X(284)-HNAKT-X(290)-
PR-X(293)-E-X(295)-FNSTFRVV-X(304)-VLTVVHQDWLNGKEY
KCKV-X(324)-N-X(326)-X(327)-X(328)-P-X(330)-P-
X(332)-X(333)-X(334)-TISKTKGQPREPQVYTLPPSREEMTKNQV
SLTCLVKGFYPSDIAVEWESNGQPENNYKTTPPMLDSDGSFFLYSKLTVD
KSRWQQGNVFSCSVMHEALHNHYTQKSLSLSPGK,

wherein
—X(221)- is selected from the group consisting of no amino acid and K;
—X(227)- is selected from the group consisting of P and G;
—X(237)- is selected from the group consisting of G and D;
—X(239)- is selected from the group consisting of S, D, E, N, Q and T;
—X(240)- is selected from the group consisting of V, I and M;
—X(246)- is selected from the group consisting of K, H and Y;
—X(255)- is selected from the group consisting of R and Y;
—X(258)- is selected from the group consisting of E, H and Y;
—X(260)- is selected from the group consisting of T and H;
—X(264)- is selected from the group consisting of V, I, T and Y;
—X(267)- is selected from the group consisting of S, D and E;
—X(268)- is selected from the group consisting of H, D and E;
—X(271)- is selected from the group consisting of P and G;
—X(272)- is selected from the group consisting of E, Y, H, R and I;
—X(274)- is selected from the group consisting of Q and E;
—X(278)- is selected from the group consisting of Y and T;
—X(281)- is selected from the group consisting of G, D and E;
—X(283)- is selected from the group consisting of E, L and H;
—X(284)- is selected from the group consisting of V, E and D;
—X(290)- is selected from the group consisting of K and N;
—X(293)- is selected from the group consisting of E and R;
—X(295)- is selected from the group consisting of Q and E;
—X(304)- is selected from the group consisting of S and T;
—X(324)- is selected from the group consisting of S, G and I;
—X(326)- is selected from the group consisting of K and T;
—X(327)- is selected from the group consisting of A, G and D;
—X(328)- is selected from the group consisting of L, A, F, I and T;
—X(330)- is selected from the group consisting of A, L, Y and I;
—X(332)- is selected from the group consisting of I, D, E, N, Q and T;
—X(333)- is selected from the group consisting of E and Y; and
—X(334)- is selected from the group consisting of K, F, I and T.

In certain variations, at least one of the positions is different from the sequence of SEQ ID NO:5. In a further variation, X(327) is A.

In another aspect, the present application is directed to an IgG2 variant including an amino acid sequence having the formula:

(SED ID NO: 95)

ASTKGPSVFPLAPCSRSTSESTAALGCLVKDYFPEPVTVSWNSGALTSGV
HTFPAVLQSSGLYSLSSVVTVPSSNFGTQTYTCNVDHKPSNTKVDKTVER
KCC-X(221)-X(222)-X(223)-X(224)-X(225)-C-X(227)-
X(228)-C-X(230)-X(231)-X(232)-X(233)-X(234)-
X(235)-X(236)-X(237)-X(238)-X(239)-X(240)-X(241)-
L-X(243)-X(244)-X(245)-X(246)-X(247)-K-X(249)-
TLMIS-X(255)-TP-X(258)-V-X(260)-C-X(262)-X(263)-
X(264)-X(265)-X(266)-X(267)-X(268)-X(269)-X(270)-
X(271)-X(272)-X(273)-X(274)-X(275)-X(276)-W-
X(278)-V-X(280)-X(281)-X(282)-X(283)-X(284)-
X(285)-X(286)-A-X(288)-T-X(290)-X(291)-X(292)-
X(293)-X(294)-X(295)-X(296)-X(297)-X(298)-X(299)-
X(300)-X(301)-X(302)-X(303)-X(304)-X(305)-LTVVHQD-
X(313)-LNG-X(317)-X(318)-Y-X(320)-C-X(322)-X(323)-
X(324)-X(325)-X(326)-X(327)-X(328)-X(329)-X(330)-
X(331)-X(332)-X(333)-X(334)-X(335)-X(336)-X(337)-
KTKGQPREPQVYTLPPSREEMTKNQVSLTCLVKGFYPSDIAVEWESNGQP
ENNYKTTPPMLDSDGSFFLYSKLTVDKSRWQQGNVFSCSVMHEALHNHYT
QKSLSLSPGK-,

wherein
—X(221)- is selected from the group consisting of no amino acid, K and Y;
—X(222)- is selected from the group consisting of V, E and Y;
—X(223)- is selected from the group consisting of no amino acid, E and K;
—X(224)- is selected from the group consisting of E and Y;
—X(225)- is selected from the group consisting of no amino acid, E, K and W;
—X(227)- is selected from the group consisting of P, E, G, K and Y;
—X(228)- is selected from the group consisting of P, E, G, K and Y;
—X(230)- is selected from the group consisting of P, A, E, G and Y;
—X(231)- is selected from the group consisting of A, E, G, K, P and Y;
—X(232)- is selected from the group consisting of P, E, G, K and Y;
—X(233)- is selected from the group consisting of P, A, D, F, G, H, I, K, L, M, N, Q, R, S, T, V, W and Y;
—X(234)- is selected from the group consisting of V, D, E, F, G, H, I, K, M, N, P, Q, R, S, T, W and Y;
—X(235)- is selected from the group consisting of A, D, F, G, H, I, K, M, N, P, Q, R, S, T, V, W and Y;
—X(236)- is selected from the group consisting of no amino acid, A, D, E, F, H, I, K, L, M, N, P, Q, R, S, T, V, W and Y;
—X(237)- is selected from the group consisting of G, D, E, F, H, I, K, L, M, N, P, Q, R, S, T, V, W and Y;
—X(238)- is selected from the group consisting of P, D, E, F, G, H, I, K, L, M, N, Q, R, S, T, V, W and Y;
—X(239)- is selected from the group consisting of S, D, E, F, G, H, I, K, L, M, N, P, Q, R, T, V, W and Y;
—X(240)- is selected from the group consisting of V, A, I, M and T;
—X(241)- is selected from the group consisting of F, D, E, L, R, S, W and Y;
—X(243)- is selected from the group consisting of F, E, H, L, Q, R, W and Y;
—X(244)- is selected from the group consisting of P and H;
—X(245)- is selected from the group consisting of P and A;
—X(246)- is selected from the group consisting of K, D, E, H and Y;
—X(247)- is selected from the group consisting of P, G and V;
—X(249)- is selected from the group consisting of D, H, Q and Y;
—X(255)- is selected from the group consisting of R, E and Y;
—X(258)- is selected from the group consisting of E, H, S and Y;
—X(260)- is selected from the group consisting of T, D, E, H and Y;
—X(262)- is selected from the group consisting of V, A, E, F, I and T;
—X(263)- is selected from the group consisting of V, A, I, M and T;
—X(264)- is selected from the group consisting of V, A, D, E, F, G, H, I, K, L, M, N, P, Q, R, S, T, W and Y;
—X(265)- is selected from the group consisting of D, F, G, H, I, K, L, M, P, Q, R, S, T, V, W and Y;
—X(266)- is selected from the group consisting of V, A, I, M and T;
—X(267)- is selected from the group consisting of S, D, E, F, H, I, K, L, M, N, P, Q, R, V, W and Y;
—X(268)- is selected from the group consisting of H, D, E, F, G, I, K, L, M, P, R, T, V and W;
—X(269)- is selected from the group consisting of E, F, G, H, I, K, L, M, N, P, R, S, T, V, W and Y;
—X(270)- is selected from the group consisting of D, F, G, H, I, L, M, P, Q, R, S, T, W and Y;
—X(271)- is selected from the group consisting of P, A, D, E, F, G, H, I, K, L, M, N, Q, R, S, T, V, W and Y;
—X(272)- is selected from the group consisting of E, D, F, G, H, I, K, L, M, P, R, S, T, V, W and Y;
—X(273)- is selected from the group consisting of V and I;
—X(274)- is selected from the group consisting of Q, D, E, F, G, H, I, L, M, N, P, R, T, V, W and Y;
—X(275)- is selected from the group consisting of F, L and W;
—X(276)- is selected from the group consisting of N, D, E, F, G, H, I, L, M, P, R, S, T, V, W and Y;
—X(278)- is selected from the group consisting of Y, D, E, G, H, I, K, L, M, N, P, Q, R, S, T, V and W;
—X(280)- is selected from the group consisting of D, G, K, L, P and W;
—X(281)- is selected from the group consisting of G, D, E, K, N, P, Q and Y;
—X(282)- is selected from the group consisting of V, E, G, K, P and Y;
—X(283)- is selected from the group consisting of E, G, H, K, L, P, R and Y;
—X(284)- is selected from the group consisting of V, D, E, L, N, Q, T and Y;
—X(285)- is selected from the group consisting of H, D, E, K, Q, W and Y;
—X(286)- is selected from the group consisting of N, E, G, P and Y;
—X(288)- is selected from the group consisting of K, D, E and Y;
—X(290)- is selected from the group consisting of K, D, H, L, N and W;
—X(291)- is selected from the group consisting of P, D, E, G, H, I, Q and T;
—X(292)- is selected from the group consisting of R, D, E, T and Y;
—X(293)- is selected from the group consisting of E, F, G, H, I, L, M, N, P, R, S, T, V, W and Y;
—X(294)- is selected from the group consisting of E, F, G, H, I, K, L, M, P, R, S, T, V, W and Y;
—X(295)- is selected from the group consisting of Q, D, E, F, G, H, I, M, N, P, R, S, T, V, W and Y;
—X(296)- is selected from the group consisting of F, A, D, E, G, I, K, L, M, N, Q, R, S, T and V;
—X(297)- is selected from the group consisting of N, D, E, F, G, H, I, K, L, M, P, Q, R, S, T, V, W and Y;
—X(298)- is selected from the group consisting of S, E, F, H, I, K, M, Q, R, W and Y;
—X(299)- is selected from the group consisting of T, A, D, E, F, G, H, I, K, L, M, N, P, Q, R, S, V, W and Y;
—X(300)- is selected from the group consisting of F, A, D, E, G, H, K, M, N, P, Q, R, S, T, V and W;
—X(301)- is selected from the group consisting of R, D, E, H and Y;
—X(302)- is selected from the group consisting of V and I;
—X(303)- is selected from the group consisting of V, D, E and Y;
—X(304)- is selected from the group consisting of S, D, H, L, N and T;
—X(305)- is selected from the group consisting of V, E, T and Y;
—X(313)- is selected from the group consisting of W and F;
—X(317)- is selected from the group consisting of K, E and Q;
—X(318)- is selected from the group consisting of E, H, L, Q, R and Y;
—X(320)- is selected from the group consisting of K, D, F, G, H, I, L, N, P, S, T, V, W and Y;
—X(322)- is selected from the group consisting of K, D, F, G, H, I, P, S, T, V, W and Y;
—X(323)- is selected from the group consisting of V and I;
—X(324)- is selected from the group consisting of S, D, F, G, H, I, L, M, P, R, T, V, W and Y;
—X(325)- is selected from the group consisting of N, A, D, E, F, G, H, I, K, L, M, P, Q, R, S, T, V, W and Y;
—X(326)- is selected from the group consisting of K, I, L, P and T;
—X(327)- is selected from the group consisting of G, D, E, F, H, I, K, L, M, N, P, R, T, V, W and Y;
—X(328)- is selected from the group consisting of L, A, D, E, F, G, H, I, K, M, N, P, Q, R, S, T, V, W and Y;
—X(329)- is selected from the group consisting of P, D, E, F, G, H, I, K, L, M, N, Q, R, S, T, V, W and Y;
—X(330)- is selected from the group consisting of A, E, F, G, H, I, L, M, N, P, R, T, V, W and Y;
—X(331)- is selected from the group consisting of P, D, F, H, I, L, M, Q, R, T, V, W and Y;
—X(332)- is selected from the group consisting of I, A, D, E, F, H, K, L, M, N, P, Q, R, S, T, V, W and Y;
—X(333)- is selected from the group consisting of E, F, H, I, L, M, P, T and Y;
—X(334)- is selected from the group consisting of K, F, I, P and T;
—X(335)- is selected from the group consisting of T, D, F, G, H, I, L, M, N, P, R, S, V, W and Y;
—X(336)- is selected from the group consisting of I, E, K and Y; and
—X(337)- is selected from the group consisting of S, E, H and N.

In certain variations, the variant differs from SEQ ID NO:11 by at least one amino acid.

In a further aspect, the present application is directed to an IgG2 variant including an amino acid sequence having the formula:

(SED ID NO: 96)

ASTKGPSVFPLAPCSRSTSESTAALGCLVKDYFPEPVTVSWNSGALTSGV
HTFPAVLQSSGLYSLSSVVTVPSSNFGTQTYTCNVDHKPSNTKVDKTVER
KCC-X(221)-V-E-C-X(227)-PCPAPP-X(234)-X(235)-
X(236)-X(237)-P-X(239)-X(240)-FLFPP-X(246)-
PKDTLMIS-X(255)-TP-X(258)-V-X(260)-CVV-X(264)-DV-
X(267)-X(268)-ED-X(271)-X(272)-V-X(274)-FNW-
X(278)-VD-X(281)-V-X(283)-X(284)-HNAKT-X(290)-PR-
X(293)-E-X(295)-FNSTFRVV-X(304)-VLTVVHQDWLNGKEYKCK
V-X(324)-N-X(326)-X(327)-X(328)-P-X(330)-P-X(332)-
X(333)-X(334)-TISKTKGQPREPQVYTLPPSREEMTKNQVSLTCLVK
GFYPSDIAVEWESNGQPENNYKTTPPMLDSDGSFFLYSKLTVDKSRWQQG
NVFSCSVMHEALHNHYTQKSLSLSPGK,

wherein
—X(221)- is selected from the group consisting of no amino acid and K;
—X(227)- is selected from the group consisting of P and G;
—X(234)- is selected from the group consisting of V, Y and I;
—X(235)- is selected from the group consisting of A, Y, I and D;
—X(236)- is selected from the group consisting of no amino acid, S and A;
—X(237)- is selected from the group consisting of G and D;
—X(239)- is selected from the group consisting of S, D, E, N, Q and T;
—X(240)- is selected from the group consisting of V, I and M;
—X(246)- is selected from the group consisting of K, H and Y;
—X(255)- is selected from the group consisting of R and Y;
—X(258)- is selected from the group consisting of E, H and Y;
—X(260)- is selected from the group consisting of T and H;
—X(264)- is selected from the group consisting of V, I, T and Y;
—X(267)- is selected from the group consisting of S, D and E;
—X(268)- is selected from the group consisting of H, D and E;
—X(271)- is selected from the group consisting of P and G;
—X(272)- is selected from the group consisting of E, Y, H, R and I;
—X(274)- is selected from the group consisting of Q and E;
—X(278)- is selected from the group consisting of Y and T;
—X(281)- is selected from the group consisting of G, D and E;
—X(283)- is selected from the group consisting of E, L and H;
—X(284)- is selected from the group consisting of V, E and D;
—X(290)- is selected from the group consisting of K and N;
—X(293)- is selected from the group consisting of E and R;
—X(295)- is selected from the group consisting of Q and E;
—X(304)- is selected from the group consisting of S and T;
—X(324)- is selected from the group consisting of S, G and I;
—X(326)- is selected from the group consisting of K and T;
—X(327)- is selected from the group consisting of G and D;
—X(328)- is selected from the group consisting of L, A, F, I and T;
—X(330)- is selected from the group consisting of A, L, Y and I;
—X(332)- is selected from the group consisting of I, D, E, N, Q and T;
—X(333)- is selected from the group consisting of E and Y; and
—X(334)- is selected from the group consisting of K, F, I and T;

In certain aspects, the variant differs from SEQ ID NO:2 by at least one amino acid.

In another aspect, the present application is directed to an IgG3 variant including two or more amino acid modifications as compared to SEQ ID NO:12. The modifications are selected from among C131S, R133K, G137E, G138S, S192N, L193F, Q196K, T199I, N203D, R214K R214T, L217P, L217R, L217S, T219S, T219C, T219Y, P220C P220G, L221D, L221-, deletion of the sequence LGD beginning at L221, T222K, T222V, deletion of T222, deletion of T223, H224E, H224P, deletion of T225, T225P, R228P, R2285, deletion of RCPEPKSCDTPPPCPRCPEPKSCDTPPPCPRCPEPKSCDTPPPCPR (SEQ ID NO: 111) beginning at 228, E233P, L234V, L234F, L235A, deletion of G236, H268Q, Q274K, K276N, Y296F, F300Y, L309V, A327G, A330S, P331S, T339A, R355Q, E356D, M358L, 5384N, N392K, M397V, K409R, Q419E, I422V, R435H, F436Y, and P445L. In certain embodiments, at least two of the amino acid modifications are in different domains. In various embodiments, the formula has at least 1, 2, 3, 4, 5, 6, 7, 8, 9, 10 or more amino acid modifications as compared to an amino acid sequence including SEQ ID NO:12. In additional embodiments, at least 2, 3, or 4 of the modifications are in different domains.

In another embodiment, the an IgG3 variant includes an amino acid sequence having the formula:

(SED ID NO: 97)

ASTKGPSVFPLAP-X(131)-S-X(133)-STSGGTAALGCLVKDYFPEP
VTVSWNSGALTSGVHTFPAVLQSSGLYSLSSVVTVPSSSLGTQTY-
X(199)-CNVNHKPSNTKVDK-X(214)-VE-X(217)-K-X(219)-
X(220)-X(221)-GD-X(222)-THTCP-X(228)-CPEPKSCDTPPPC
PRCPEPKSCDTPPPCPRCPEPKSCDTPPPCPRCPAPELLGGPSVFLFPPK
PKDTLMISRTPEVTCVVVDVSHEDPEV-X(274)-F-X(276)-WYVDGV
EVHNAKTKPREEQYNST-X(300)-RVVSVLTVLHQDWLNGKEYKCKVSN
KALPAPIEKTISK-X(339)-KGQPREPQVYTLPPSR-X(356)-E-
X(358)-TKNQVSLTCLVKGFYPSDAVEWES-X(384)-GQPENNY-
X(392)-TTPP-X(397)-LDSDGSFFLYSKLTVDKSRWQQGN-
X(422)-FSCSVMHEALHN-X(435)-X(436)-TQKSLSLSPGK,

wherein
X(131) is selected from the group consisting of C and S;
X(133) is selected from the group consisting of R and K;
X(199) is selected from the group consisting of T and I;
X(214) is selected from the group consisting of R and K;
X(217) is selected from the group consisting of L and P;
X(219) is selected from the group consisting of T and S;
X(220) is selected from the group consisting of P and C;
X(221) is selected from the group consisting of D L, and the sequence LGD;
X(222) is selected from the group consisting of T and K;
X(228) is selected from the group consisting of R, the sequence

(SEQ ID NO: 111)

RCPEPKSCDTPPPCPRCPEPKSCDTPPPCPRCPEPKSCDTPPPCPR

### and P;

X(274) is selected from the group consisting of Q and K;
X(276) is selected from the group consisting of K and N;
X(300) is selected from the group consisting of F and Y;
X(339) is selected from the group consisting of T and A;
X(356) is selected from the group consisting of E and D;
X(358) is selected from the group consisting of M and L;
X(384) is selected from the group consisting of S and N;
X(392) is selected from the group consisting of N and K;
X(397) is selected from the group consisting of M and V;
X(422) is selected from the group consisting of I and V;
X(435) is selected from the group consisting of R and H; and
X(436) is selected from the group consisting of F and Y.

In certain variations, the formula has at least two amino acid modifications as compared to SEQ ID NO:12. In further variations, the two of modifications can in different domains. In various embodiments, the formula has at least 1, 2, 3, 4, 5, 6, 7, 8, 9, 10 or more amino acid modifications as compared to an amino acid sequence including SEQ ID NO:12. In additional embodiments, at least 2, 3, or 4 of the modifications are in different domains.

In another aspect, the present application is directed to an IgG3 variant including two or more amino acid modifications as compared to SEQ ID NO:12. The modifications can be selected from among C131S, R133K, G137E, G138S, S192N, L193F, Q196K, T199I, N203D, R214K R214T, L217P, L217R, L217S, T219S, T219C, T219Y, P220C P220G, L221D, the deletion of L221, deletion of GD, T222K, T222V, the deletion of T222, the deletion of T223, H224E, H224P, the deletion of T225, T225P, R228P, R2285, deletion of RCPEPKSCDTPPPCPRCPEPKSCDTPPPCPRCPEPKSCDTPPPCPR (SEQ ID NO: 111) beginning at R228, E233P, L234V, L234F, L235A, G236-, H268Q, Q274K, K276N, Y296F, F300Y, L309V, A327G, A330S, P331S, T339A, R355Q, E356D, M358L, 5384N, N392K, M397V, K409R, Q419E, I422V, R435H, F436Y, and P445L. In certain embodiments, at least two of the amino acid modifications are in different domains. In various embodiments, the formula has at least 1, 2, 3, 4, 5, 6, 7, 8, 9, 10 or more amino acid modifications as compared to an amino acid sequence including SEQ ID NO:11. In additional embodiments, at least 2, 3, or 4 of the modifications are in different domains.

In another aspect, the present application is directed to an IgG3 variant including an amino acid sequence having the formula:

(SED ID NO: 98)
-ASTKGPSVFPLAP-X(131)-S-X(133)-STS-X(137)-X(138)-
TAALGCLVKDYFPEPVTVSWNSGALTSGVHTFPAVLQSSGLYSLSSVVTVPSS-
X(192)-X(193)-GT-X(196)-TY-X(199)-CNV-X(203)-HKPSNTKVDK-X(214)-VE-X(217)-
K-X(219)-X(220)-X(221)-X(222)-X(223)-X(224)-X(225)-C-X(227)-X(228)-C-X(230)-
X(231)-X(232)-X(233)-X(234)-X(235)-X(236)-X(237)-X(238)-X(239)-X(240)-X(241)-L-
X(243)-X(244)-X(245)-X(246)-X(247)-K-X(249)-TLMIS-X(255)-TP-X(258)-V-X(260)-C-
X(262)-X(263)-X(264)-X(265)-X(266)-X(267)-X(268)-X(269)-X(270)-X(271)-X(272)-
X(273)-X(274)-X(275)-X(276)-W-X(278)-V-X(280)-X(281)-X(282)-X(283)-X(284)-
X(285)-X(286)-A-X(288)-T-X(290)-X(291)-X(292)-X(293)-X(294)-X(295)-X(296)-X(297)-
X(298)-X(299)-X(300)-X(301)-X(302)-X(303)-X(304)-X(305)-LTV-X(309)-HQD-X(313)-
LNG-X(317)-X(318)-Y-X(320)-C-X(322)-X(323)-X(324)-X(325)-X(326)-X(327)-X(328)-
X(329)-X(330)-X(331)-X(332)-X(333)-X(334)-X(335)-X(336)-X(337)-K-X(339)-
KGQPREPQVYTLPPS-X(355)-X(356)-E-X(358)-TKNQVSLTCLVKGFYPSDIAVEWES-
X(384)-GQPENNY-X(392)-TTPP-X(397)-LDSDGSFFLYS-X(409)-LTVDKSRWQ-
X(419)-GN-X(422)-FSCSVMHEALHN-X(435)-X(436)-TQKSLSLS-X(445)-GK,

wherein
—X(131)- is selected from the group consisting of C and S;
—X(133)- is selected from the group consisting of R and K;
—X(137)- is selected from the group consisting of E and G;
—X(138)- is selected from the group consisting of S and G;
—X(192)- is selected from the group consisting of N and S;
—X(193)- is selected from the group consisting of F and L;
—X(196)- is selected from the group consisting of Q and K;
—X(199)- is selected from the group consisting of T and I;
—X(203)- is selected from the group consisting of D and N;
—X(214)- is selected from the group consisting of T, K and R;
—X(217)- is selected from the group consisting of R, P, L and S;
—X(219)- is selected from the group consisting of C, S, T and Y;
—X(220)- is selected from the group consisting of C, P and G;
—X(221)- is selected from the group consisting of no amino acid, D, K, Y, L, and the sequence LGD;
—X(222)- is selected from the group consisting of V, K, T, no amino acid, E and Y;
—X(223)- is selected from the group consisting of no amino acid, T, E and K;
—X(224)- is selected from the group consisting of E, H, P and Y;
—X(225)- is selected from the group consisting of no amino acid, T, P, E, K and W;
—X(227)- is selected from the group consisting of P, E, G, K and Y;
—X(228)- is selected from the group consisting of P, S, E, G, K, Y, R, and the sequence

(SEQ ID NO: 111)

RCPEPKSCDTPPPCPRCPEPKSCDTPPPCPRCPEPKSCDTPPPCPR;

—X(230)- is selected from the group consisting of P, A, E, G and Y;
—X(231)- is selected from the group consisting of A, E, G, K, P and Y;
—X(232)- is selected from the group consisting of P, E, G, K and Y;
—X(233)- is selected from the group consisting of P, E, A, D, F, G, H, I, K, L, M, N, Q, R, S, T, V, W and Y;
—X(234)- is selected from the group consisting of V, L, F, D, E, F, G, H, I, K, M, N, P, Q, R, S, T, W and Y;
—X(235)- is selected from the group consisting of A, L, D, F, G, H, I, K, M, N, P, Q, R, S, T, V, W, and Y;
—X(236)- is selected from the group consisting of no amino acid, G, A, D, E, F, H, I, K, L, M, N, P, Q, R, S, T, V, W and Y;
—X(237)- is selected from the group consisting of G, D, E, F, H, I, K, L, M, N, P, Q, R, S, T, V, W and Y;
—X(238)- is selected from the group consisting of P, D, E, F, G, H, I, K, L, M, N, Q, R, S, T, V, W and Y;
—X(239)- is selected from the group consisting of S, D, E, F, G, H, I, K, L, M, N, P, Q, R, T, V, W and Y;
—X(240)- is selected from the group consisting of V, A, I, M and T;
—X(241)- is selected from the group consisting of F, D, E, L, R, S, W and Y;
—X(243)- is selected from the group consisting of F, E, H, L, Q, RW, and Y;
—X(244)- is selected from the group consisting of P and H;
—X(245)- is selected from the group consisting of P and A;
—X(246)- is selected from the group consisting of K, D, E, H and Y;
—X(247)- is selected from the group consisting of P, G and V;
—X(249)- is selected from the group consisting of D, H, Q and Y;
—X(255)- is selected from the group consisting of RE and Y;
—X(258)- is selected from the group consisting of E, H, S and Y;
—X(260)- is selected from the group consisting of T, D, E, H and Y;
—X(262)- is selected from the group consisting of V, A, E, F, I and T;
—X(263)- is selected from the group consisting of V, A, I, M and T;
—X(264)- is selected from the group consisting of V, A, D, E, F, G, H, I, K, L, M, N, P, Q, R, S, T, W, and Y;
—X(265)- is selected from the group consisting of D, F, G, H, I, K, L, M, P, Q, R, S, T, V, W and Y;
—X(266)- is selected from the group consisting of V, A, I, M and T;
—X(267)- is selected from the group consisting of S, D, E, F, H, I, K, L, M, N, P, Q, R, V, W and Y;
—X(268)- is selected from the group consisting of H, Q, D, E, F, G, I, K, L, M, P, R, T, V and W;
—X(269)- is selected from the group consisting of E, F, G, H, I, K, L, M, N, P, R, S, T, V, W and Y;
—X(270)- is selected from the group consisting of D, F, G, H, I, L, M, P, Q, R, S, T, W and Y;
—X(271)- is selected from the group consisting of P, A, D, E, F, G, H, I, K, L, M, N, Q, R, S, T, V, W and Y;
—X(272)- is selected from the group consisting of E, D, F, G, H, I, K, L, M, P, R, S, T, V, W and Y;
—X(273)- is selected from the group consisting of V and I;
—X(274)- is selected from the group consisting of Q, K, D, E, F, G, H, I, L, M, N, P, R, T, V, W and Y;
—X(275)- is selected from the group consisting of FL and W;
—X(276)- is selected from the group consisting of N, K, D, E, F, G, H, I, L, M, P, R, S, T, V, W and Y;
—X(278)- is selected from the group consisting of Y, D, E, G, H, I, K, L, M, N, P, Q, R, S, T, V and W;
—X(280)- is selected from the group consisting of D, G, K, L, P and W;
—X(281)- is selected from the group consisting of G, D, E, K, N, P, Q and Y;
—X(282)- is selected from the group consisting of V, E, G, K, P and Y;
—X(283)- is selected from the group consisting of E, G, H, K, L, P, R and Y;
—X(284)- is selected from the group consisting of V, D, E, L, N, Q, T and Y;
—X(285)- is selected from the group consisting of H, D, E, K, Q, W and Y;
—X(286)- is selected from the group consisting of N, E, G, P and Y;
—X(288)- is selected from the group consisting of K, D, E and Y;
—X(290)- is selected from the group consisting of K, D, H, L, N and W;
—X(291)- is selected from the group consisting of P, D, E, G, H, I, Q and T;
—X(292)- is selected from the group consisting of R, D, E, T and Y;
—X(293)- is selected from the group consisting of E, F, G, H, I, L, M, N, P, R, S, T, V, W and Y;
—X(294)- is selected from the group consisting of E, F, G, H, I, K, L, M, P, R, S, T, V, W and Y;
—X(295)- is selected from the group consisting of Q, D, E, F, G, H, I, M, N, P, R, S, T, V, W and Y;
—X(296)- is selected from the group consisting of F, Y, A, D, E, G, I, K, L, M, N, Q, R, S, T and V;
—X(297)- is selected from the group consisting of N, D, E, F, G, H, I, K, L, M, P, Q, R, S, T, V, W and Y;
—X(298)- is selected from the group consisting of S, E, F, H, I, K, M, Q, R, W and Y;
—X(299)- is selected from the group consisting of T, A, D, E, F, G, H, I, K, L, M, N, P, Q, R, S, V, W and Y;
—X(300)- is selected from the group consisting of F, Y, A, D, E, G, H, K, M, N, P, Q, R, S, T, V and W;
—X(301)- is selected from the group consisting of R, D, E, H and Y;
—X(302)- is selected from the group consisting of V and I;
—X(303)- is selected from the group consisting of V, D, E and Y;
—X(304)- is selected from the group consisting of S, D, H, L, N and T;
—X(305)- is selected from the group consisting of V, E, T and Y;
—X(309)- is selected from the group consisting of V and L;
—X(313)- is selected from the group consisting of W and F;
—X(317)- is selected from the group consisting of K, E and Q;
—X(318)- is selected from the group consisting of E, H, L, Q, R and Y;
—X(320)- is selected from the group consisting of K, D, F, G, H, I, L, N, P, S, T, V, W and Y;
—X(322)- is selected from the group consisting of K, D, F, G, H, I, P, S, T, V, W and Y;
—X(323)- is selected from the group consisting of V and I;
—X(324)- is selected from the group consisting of S, D, F, G, H, I, L, M, P, R, T, V, W and Y;
—X(325)- is selected from the group consisting of N, A, D, E, F, G, H, I, K, L, M, P, Q, R, S, T, V, W and Y;
—X(326)- is selected from the group consisting of K, I, L, P and T;
—X(327)- is selected from the group consisting of G, A, D, E, F, H, I, K, L, M, N, P, R, T, V, W and Y;
—X(328)- is selected from the group consisting of L, A, D, E, F, G, H, I, K, M, N, P, Q, R, S, T, V, W and Y;
—X(329)- is selected from the group consisting of P, D, E, F, G, H, I, K, L, M, N, Q, R, S, T, V, W and Y;
—X(330)- is selected from the group consisting of A, S, E, F, G, H, I, L, M, N, P, R, T, V, W and Y;
—X(331)- is selected from the group consisting of P, S, D, F, H, I, L, M, Q, R, T, V, W and Y;
—X(332)- is selected from the group consisting of I, A, D, E, F, H, K, L, M, N, P, Q, R, S, T, V, W and Y;
—X(333)- is selected from the group consisting of E, F, H, I, L, M, P, T and Y;
—X(334)- is selected from the group consisting of K, F, I, P and T;
—X(335)- is selected from the group consisting of T, D, F, G, H, I, L, M, N, P, R, S, V, W and Y;
—X(336)- is selected from the group consisting of I, E, K and Y;
—X(337)- is selected from the group consisting of S, E, H and N;
—X(339)- is selected from the group consisting of T and A;
—X(355)- is selected from the group consisting of R and Q;
—X(356)- is selected from the group consisting of E and D;
—X(358)- is selected from the group consisting of M and L;
—X(384)- is selected from the group consisting of N and S;
—X(392)- is selected from the group consisting of K and N;
—X(397)- is selected from the group consisting of M and V;
—X(409)- is selected from the group consisting of K and R;
—X(419)- is selected from the group consisting of Q and E;
—X(422)- is selected from the group consisting of V and I;
—X(435)- is selected from the group consisting of H and R;
—X(436)- is selected from the group consisting of Y and F; and
—X(445)- is selected from the group consisting of P and L.

In one variation, a first modification can be selected from among C131S, R133K, G137E, G138S, S192N, L193F, Q196K, T199I, N203D, R214K R214T, L217P, L217R, L217S, T219S, T219C, T219Y, P220C P220G, L221D, deletion of L221, deletion of the sequence LGD beginning at L221, T222K, T222V, deletion of T222, deletion of T223, H224E, H224P, deletion of T225, T225P, R228P, R228S, deletion of the sequence

(SEQ ID NO: 111)

RCPEPKSCDTPPPCPRCPEPKSCDTPPPCPRCPEPKSCDTPPPCPR

beginning at 228, E233P, L234V, L234F, L235A, deletion of G236, H268Q, Q274K, K276N, Y296F, F300Y, L309V, A327G, A330S, P331S, T339A, R355Q, E356D, M358L, S384N, N392K, M397V, K409R, Q419E, I422V, R435H, F436Y, and P445L. In a further variation, a second modification is selected from among 221K, 221Y, 222E, 222Y, 223E, 223K, 224Y, 225E, 225K, 225W, 227E, 227G, 227K, 227Y, 228E, 228G, 228K, 228Y, 230A, 230E, 230G, 230Y, 231E, 231G, 231K, 231P, 231Y, 232E, 232G, 232K, 232Y, 233A, 233D, 233F, 233G, 233H, 233I, 233K, 233L, 233M, 233N, 233Q, 233R, 233S, 233T, 233V, 233W, 233Y, 234D, 234E, 234F, 234G, 234H, 234I, 234K, 234M, 234N, 234P, 234Q, 234R, 234S, 234T, 234W, 234Y, 235D, 235F, 235G, 235H, 235I, 235K, 235M, 235N, 235P, 235Q, 235R, 235S, 235T, 235V, 235W, 235Y, 236A, 236D, 236E, 236F, 236H, 236I, 236K, 236L, 236M, 236N, 236P, 236Q, 236R, 236S, 236T, 236V, 236W, 236Y, 237D, 237E, 237F, 237H, 237I, 237K, 237L, 237M, 237N, 237P, 237Q, 237R, 237S, 237T, 237V, 237W, 237Y, 238D, 238E, 238F, 238G, 238H, 238I, 238K, 238L, 238M, 238N, 238Q, 238R, 238S, 238T, 238V, 238W, 238Y, 239D, 239E, 239F, 239G, 239H, 239I, 239K, 239L, 239M, 239N, 239P, 239Q, 239R, 239T, 239V, 239W, 239Y, 240A, 240I, 240M, 240T, 241D, 241E, 241L, 241R, 241S, 241W, 241Y, 243E, 243H, 243L, 243Q, 243R, 243W, 243Y, 244H, 245A, 246D, 246E, 246H, 246Y, 247G, 247V, 249H, 249Q, 249Y, 255E, 255Y, 258H, 258S, 258Y, 260D, 260E, 260H, 260Y, 262A, 262E, 262F, 262I, 262T, 263A, 263I, 263M, 263T, 264A, 264D, 264E, 264F, 264G, 264H, 264I, 264K, 264L, 264M, 264N, 264P, 264Q, 264R, 264S, 264T, 264W, 264Y, 265F, 265G, 265H, 265I, 265K, 265L, 265M, 265P, 265Q, 265R, 265S, 265T, 265V, 265W, 265Y, 266A, 266I, 266M, 266T, 267D, 267E, 267F, 267H, 267I, 267K, 267L, 267M, 267N, 267P, 267Q, 267R, 267V, 267W, 267Y, 268D, 268E, 268F, 268G, 268I, 268K, 268L, 268M, 268P, 268R, 268T, 268V, 268W, 269F, 269G, 269H, 269I, 269K, 269L, 269M, 269N, 269P, 269R, 269S, 269T, 269V, 269W, 269Y, 270F, 270G, 270H, 270I, 270L, 270M, 270P, 270Q, 270R, 270S, 270T, 270W, 270Y, 271A, 271D, 271E, 271F, 271G, 271H, 271I, 271K, 271L, 271M, 271N, 271Q, 271R, 271S, 271T, 271V, 271W, 271Y, 272D, 272F, 272G, 272H, 272I, 272K, 272L, 272M, 272P, 272R, 272S, 272T, 272V, 272W, 272Y, 273I, 274D, 274E, 274F, 274G, 274H, 274I, 274L, 274M, 274N, 274P, 274R, 274T, 274V, 274W, 274Y, 275L, 275W, 276D, 276E, 276F, 276G, 276H, 276I, 276L, 276M, 276P, 276R, 276S, 276T, 276V, 276W, 276Y, 278D, 278E, 278G, 278H, 278I, 278K, 278L, 278M, 278N, 278P, 278Q, 278R, 278S, 278T, 278V, 278W, 280G, 280K, 280L, 280P, 280W, 281D, 281E, 281K, 281N, 281P, 281Q, 281Y, 282E, 282G, 282K, 282P, 282Y, 283G, 283H, 283K, 283L, 283P, 283R, 283Y, 284D, 284E, 284L, 284N, 284Q, 284T, 284Y, 285D, 285E, 285K, 285Q, 285W, 285Y, 286E, 286G, 286P, 286Y, 288D, 288E, 288Y, 290D, 290H, 290L, 290N, 290W, 291D, 291E, 291G, 291H, 291I, 291Q, 291T, 292D, 292E, 292T, 292Y, 293F, 293G, 293H, 293I, 293L, 293M, 293N, 293P, 293R, 293S, 293T, 293V, 293W, 293Y, 294F, 294G, 294H, 294I, 294K, 294L, 294M, 294P, 294R, 294S, 294T, 294V, 294W, 294Y, 295D, 295E, 295F, 295G, 295H, 295I, 295M, 295N, 295P, 295R, 295S, 295T, 295V, 295W, 295Y, 296A, 296D, 296E, 296G, 296I, 296K, 296L, 296M, 296N, 296Q, 296R, 296S, 296T, 296V, 297D, 297E, 297F, 297G, 297H, 297I, 297K, 297L, 297M, 297P, 297Q, 297R, 297S, 297T, 297V, 297W, 297Y, 298E, 298F, 298H, 298I, 298K, 298M, 298Q, 298R, 298W, 298Y, 299A, 299D, 299E, 299F, 299G, 299H, 299I, 299K, 299L, 299M, 299N, 299P, 299Q, 299R, 299S, 299V, 299W, 299Y, 300A, 300D, 300E, 300G, 300H, 300K, 300M, 300N, 300P, 300Q, 300R, 300S, 300T, 300V, 300W, 301D, 301E, 301H, 301Y, 302I, 303D, 303E, 303Y, 304D, 304H, 304L, 304N, 304T, 305E, 305T, 305Y, 313F, 317E, 317Q, 318H, 318L, 318Q, 318R, 318Y, 320D, 320F, 320G, 320H, 320I, 320L, 320N, 320P, 320S, 320T, 320V, 320W, 320Y, 322D, 322F, 322G, 322H, 322I, 322P, 322S, 322T, 322V, 322W, 322Y, 323I, 324D, 324F, 324G, 324H, 324I, 324L, 324M, 324P, 324R, 324T, 324V, 324W, 324Y, 325A, 325D, 325E, 325F, 325G, 325H, 325I, 325K, 325L, 325M, 325P, 325Q, 325R, 325S, 325T, 325V, 325W, 325Y, 326I, 326L, 326P, 326T, 327D, 327E, 327F, 327H, 327I, 327K, 327L, 327M, 327N, 327P, 327R, 327T, 327V, 327W, 327Y, 328A, 328D, 328E, 328F, 328G, 328H, 328I, 328K, 328M, 328N, 328P, 328Q, 328R, 328S, 328T, 328V, 328W, 328Y, 329D, 329E, 329F, 329G, 329H, 329I, 329K, 329L, 329M, 329N, 329Q, 329R, 329S, 329T, 329V, 329W, 329Y, 330E, 330F, 330G, 330H, 330I, 330L, 330M, 330N, 330P, 330R, 330T, 330V, 330W, 330Y, 331D, 331F, 331H, 331I, 331L, 331M, 331Q, 331R, 331T, 331V, 331W, 331Y, 332A, 332D, 332E, 332F, 332H, 332K, 332L, 332M, 332N, 332P, 332Q, 332R, 332S, 332T, 332V, 332W, 332Y, 333F, 333H, 333I, 333L, 333M, 333P, 333T, 333Y, 334F, 334I, 334P, 334T, 335D, 335F, 335G, 335H, 335I, 335L, 335M, 335N, 335P, 335R, 335S, 335V, 335W, 335Y, 336E, 336K, 336Y, 337E, 337H, 337N. In various embodiments, the formula has at least 1, 2, 3, 4, 5, 6, 7, 8, 9, 10 or more amino acid modifications as compared to an amino acid sequence including SEQ ID NO:12. In additional embodiments, at least 2, 3, or 4 of the modifications are in different domains.

In a further aspect, the present application is directed to an IgG3 variant amino acid sequence having at least two amino acid modifications as compared to SEQ ID NO:13, wherein a first modification is selected from among C131S, R133K, G137E, G138S, S192N, L193F, Q196K, T199I, N203D, R214K R214T, L217P, L217R, L217S, T219S, T219C, T219Y, P220C P220G, L221D, deletion of L221, deletion of the sequence LGD beginning at L221, T222K, T222V, deletion of T222, deletion of T223, H224E, H224P, deletion of T225, T225P, R228P, R2285, deletion of the sequence

(SEQ ID NO: 111)

RCPEPKSCDTPPPCPRCPEPKSCDTPPPCPRCPEPKSCDTPPPCPR

beginning at 228, E233P, L234V, L234F, L235A, deletion of G236, H268Q, Q274K, K276N, Y296F, F300Y, L309V, A327G, A330S, P331S, T339A, R355Q, E356D, M358L, 5384N, N392K, M397V, K409R, Q419E, I422V, R435H, F436Y, and P445L, and a second modification is selected from among 221K, 221Y, 222E, 222Y, 223E, 223K, 224Y, 225E, 225K, 225W, 227E, 227G, 227K, 227Y, 228E, 228G, 228K, 228Y, 230A, 230E, 230G, 230Y, 231E, 231G, 231K, 231P, 231Y, 232E, 232G, 232K, 232Y, 233A, 233D, 233F, 233G, 233H, 233I, 233K, 233L, 233M, 233N, 233Q, 233R, 233S, 233T, 233V, 233W, 233Y, 234D, 234E, 234F, 234G, 234H, 234I, 234K, 234M, 234N, 234P, 234Q, 234R, 234S, 234T, 234W, 234Y, 235D, 235F, 235G, 235H, 235I, 235K, 235M, 235N, 235P, 235Q, 235R, 235S, 235T, 235V, 235W, 235Y, 236A, 236D, 236E, 236F, 236H, 236I, 236K, 236L, 236M, 236N, 236P, 236Q, 236R, 236S, 236T, 236V, 236W, 236Y, 237D, 237E, 237F, 237H, 237I, 237K, 237L, 237M, 237N, 237P, 237Q, 237R, 237S, 237T, 237V, 237W, 237Y, 238D, 238E, 238F, 238G, 238H, 238I, 238K, 238L, 238M, 238N, 238Q, 238R, 238S, 238T, 238V, 238W, 238Y, 239D, 239E, 239F, 239G, 239H, 239I, 239K, 239L, 239M, 239N, 239P, 239Q, 239R, 239T, 239V, 239W, 239Y, 240A, 240I, 240M, 240T, 241D, 241E, 241L, 241R, 241S, 241W, 241Y, 243E, 243H, 243L, 243Q, 243R, 243W, 243Y, 244H, 245A, 246D, 246E, 246H, 246Y, 247G, 247V, 249H, 249Q, 249Y, 255E, 255Y, 258H, 258S, 258Y, 260D, 260E, 260H, 260Y, 262A, 262E, 262F, 262I, 262T, 263A, 263I, 263M, 263T, 264A, 264D, 264E, 264F, 264G, 264H, 264I, 264K, 264L, 264M, 264N, 264P, 264Q, 264R, 264S, 264T, 264W, 264Y, 265F, 265G, 265H, 265I, 265K, 265L, 265M, 265P, 265Q, 265R, 265S, 265T, 265V, 265W, 265Y, 266A, 266I, 266M, 266T, 267D, 267E, 267F, 267H, 267I, 267K, 267L, 267M, 267N, 267P, 267Q, 267R, 267V, 267W, 267Y, 268D, 268E, 268F, 268G, 268I, 268K, 268L, 268M, 268P, 268R, 268T, 268V, 268W, 269F, 269G, 269H, 269I, 269K, 269L, 269M, 269N, 269P, 269R, 269S, 269T, 269V, 269W, 269Y, 270F, 270G, 270H, 270I, 270L, 270M, 270P, 270Q, 270R, 270S, 270T, 270W, 270Y, 271A, 271D, 271E, 271F, 271G, 271H, 271I, 271K, 271L, 271M, 271N, 271Q, 271R, 271S, 271T, 271V, 271W, 271Y, 272D, 272F, 272G, 272H, 272I, 272K, 272L, 272M, 272P, 272R, 272S, 272T, 272V, 272W, 272Y, 273I, 274D, 274E, 274F, 274G, 274H, 274I, 274L, 274M, 274N, 274P, 274R, 274T, 274V, 274W, 274Y, 275L, 275W, 276D, 276E, 276F, 276G, 276H, 276I, 276L, 276M, 276P, 276R, 276S, 276T, 276V, 276W, 276Y, 278D, 278E, 278G, 278H, 278I, 278K, 278L, 278M, 278N, 278P, 278Q, 278R, 278S, 278T, 278V, 278W, 280G, 280K, 280L, 280P, 280W, 281D, 281E, 281K, 281N, 281P, 281Q, 281Y, 282E, 282G, 282K, 282P, 282Y, 283G, 283H, 283K, 283L, 283P, 283R, 283Y, 284D, 284E, 284L, 284N, 284Q, 284T, 284Y, 285D, 285E, 285K, 285Q, 285W, 285Y, 286E, 286G, 286P, 286Y, 288D, 288E, 288Y, 290D, 290H, 290L, 290N, 290W, 291D, 291E, 291G, 291H, 291I, 291Q, 291T, 292D, 292E, 292T, 292Y, 293F, 293G, 293H, 293I, 293L, 293M, 293N, 293P, 293R, 293S, 293T, 293V, 293W, 293Y, 294F, 294G, 294H, 294I, 294K, 294L, 294M, 294P, 294R, 294S, 294T, 294V, 294W, 294Y, 295D, 295E, 295F, 295G, 295H, 295I, 295M, 295N, 295P, 295R, 295S, 295T, 295V, 295W, 295Y, 296A, 296D, 296E, 296G, 296I, 296K, 296L, 296M, 296N, 296Q, 296R, 296S, 296T, 296V, 297D, 297E, 297F, 297G, 297H, 297I, 297K, 297L, 297M, 297P, 297Q, 297R, 297S, 297T, 297V, 297W, 297Y, 298E, 298F, 298H, 298I, 298K, 298M, 298Q, 298R, 298W, 298Y, 299A, 299D, 299E, 299F, 299G, 299H, 299I, 299K, 299L, 299M, 299N, 299P, 299Q, 299R, 299S, 299V, 299W, 299Y, 300A, 300D, 300E, 300G, 300H, 300K, 300M, 300N, 300P, 300Q, 300R, 300S, 300T, 300V, 300W, 301D, 301E, 301H, 301Y, 302I, 303D, 303E, 303Y, 304D, 304H, 304L, 304N, 304T, 305E, 305T, 305Y, 313F, 317E, 317Q, 318H, 318L, 318Q, 318R, 318Y, 320D, 320F, 320G, 320H, 320I, 320L, 320N, 320P, 320S, 320T, 320V, 320W, 320Y, 322D, 322F, 322G, 322H, 322I, 322P, 322S, 322T, 322V, 322W, 322Y, 323I, 324D, 324F, 324G, 324H, 324I, 324L, 324M, 324P, 324R, 324T, 324V, 324W, 324Y, 325A, 325D, 325E, 325F, 325G, 325H, 325I, 325K, 325L, 325M, 325P, 325Q, 325R, 325S, 325T, 325V, 325W, 325Y, 326I, 326L, 326P, 326T, 327D, 327E, 327F, 327H, 327I, 327K, 327L, 327M, 327N, 327P, 327R, 327T, 327V, 327W, 327Y, 328A, 328D, 328E, 328F, 328G, 328H, 328I, 328K, 328M, 328N, 328P, 328Q, 328R, 328S, 328T, 328V, 328W, 328Y, 329D, 329E, 329F, 329G, 329H, 329I, 329K, 329L, 329M, 329N, 329Q, 329R, 329S, 329T, 329V, 329W, 329Y, 330E, 330F, 330G, 330H, 330I, 330L, 330M, 330N, 330P, 330R, 330T, 330V, 330W, 330Y, 331D, 331F, 331H, 331I, 331L, 331M, 331Q, 331R, 331T, 331V, 331W, 331Y, 332A, 332D, 332E, 332F, 332H, 332K, 332L, 332M, 332N, 332P, 332Q, 332R, 332S, 332T, 332V, 332W, 332Y, 333F, 333H, 333I, 333L, 333M, 333P, 333T, 333Y, 334F, 334I, 334P, 334T, 335D, 335F, 335G, 335H, 335I, 335L, 335M, 335N, 335P, 335R, 335S, 335V, 335W, 335Y, 336E, 336K, 336Y, 337E, 337H, and 337N. In various embodiments, the formula has at least 1, 2, 3, 4, 5, 6, 7, 8, 9, 10 or more amino acid modifications as compared to an amino acid sequence including SEQ ID NO:12. In additional embodiments, at least 2, 3, or 4 of the modifications are in different domains.

In another aspect, the present application is directed to an IgG3 variant including an amino acid sequence having the formula:

(SED ID NO: 99)
ASTKGPSVFPLAP-X(131)-S-X(133)-STS-X(137)-X(138)-
TAALGCLVKDYFPEPVTVSWNSGALTSGVHTFPAVLQSSGLYSLSSVVTVPSS-
X(192)-X(193)-GT-X(196)-TY-X(199)-CNV-X(203)-HKPSNTKVDK-X(214)-VE-X(217)-
K-X(219)-X(220)-X(221)-X(222)-X(223)-X(224)-X(225)-C-X(227)-X(228)-CPAP-X(233)-
X(234)-X(235)-X(236)-X(237)-P-X(239)-X(240)-FLFPP-X(246)-PKDTLMIS-X(255)-TP-
X(258)-V-X(260)-CVV-X(264)-DV-X(267)-X(268)-ED-X(271)-X(272)-V-X(274)-F-
X(276)-W-X(278)-VD-X(281)-V-X(283)-X(284)-HNAKT-X(290)-PR-X(293)-E-X(295)-
X(296)-NST-X(300)-RVV-X(304)-VLTV-X(309)-HQDWLNGKEYKCKV-X(324)-N-
X(326)-X(327)-X(328)-P-X(330)-X(331)-X(332)-X(333)-X(334)-TISK-X(339)-
KGQPREPQVYTLPPS-X(355)-X(356)-E-X(358)-TKNQVSLTCLVKGFYPSDIAVEWES-
X(384)-GQPENNY-X(392)-TTPP-X(397)-LDSDGSFFLYS-X(409)-LTVDKSRWQ-
X(419)-GN-X(422)-FSCSVMHEALHN-X(435)-X(436)-TQKSLSLS-X(445)-GK;

wherein
—X(131)- is selected from the group consisting of C and S;
—X(133)- is selected from the group consisting of R and K;
—X(137)- is selected from the group consisting of E and G;
—X(138)- is selected from the group consisting of S and G;
—X(192)- is selected from the group consisting of N and S;
—X(193)- is selected from the group consisting of F and L;
—X(196)- is selected from the group consisting of Q and K;
—X(199)- is selected from the group consisting of T and I;
—X(203)- is selected from the group consisting of D and N;
—X(214)- is selected from the group consisting of T, K and R;
—X(217)- is selected from the group consisting of R, P, L and S;
—X(219)- is selected from the group consisting of C, S, T and Y;
—X(220)- is selected from the group consisting of C, P and G;
—X(221)- is selected from the group consisting of no amino acid, D, L, K, and the sequence LGD;
—X(222)- is selected from the group consisting of V, K, T, and no amino acid;
—X(223)- is selected from the group consisting of no amino acid and T;
—X(224)- is selected from the group consisting of E, H and P;
—X(225)- is selected from the group consisting of no amino acid, T and P;
—X(227)- is selected from the group consisting of P and G;
—X(228)- is selected from the group consisting of P, R, S, and the sequence

(SEQ ID NO: 111)

RCPEPKSCDTPPPCPRCPEPKSCDTPPPCPRCPEPKSCDTPPPCPR;

—X(233)- is selected from the group consisting of P and E;
—X(234)- is selected from the group consisting of V, L, F, Y and I;
—X(235)- is selected from the group consisting of A, L, Y, I and D;
—X(236)- is selected from the group consisting of no amino acid, G, S and A;
—X(237)- is selected from the group consisting of G and D;
—X(239)- is selected from the group consisting of S, D, E, N, Q and T;
—X(240)- is selected from the group consisting of V, I and M;
—X(246)- is selected from the group consisting of K, H and Y;
—X(255)- is selected from the group consisting of R and Y;
—X(258)- is selected from the group consisting of E, H and Y;
—X(260)- is selected from the group consisting of T and H;
—X(264)- is selected from the group consisting of V, I, T and Y;
—X(267)- is selected from the group consisting of S, D and E;
—X(268)- is selected from the group consisting of H, Q, D and E;
—X(271)- is selected from the group consisting of P and G;
—X(272)- is selected from the group consisting of E, Y, H, R and I;
—X(274)- is selected from the group consisting of Q, K and E;
—X(276)- is selected from the group consisting of N and K;
—X(278)- is selected from the group consisting of Y and T;
—X(281)- is selected from the group consisting of G, D and E;
—X(283)- is selected from the group consisting of E, L and H;
—X(284)- is selected from the group consisting of V, E and D;
—X(290)- is selected from the group consisting of K and N;
—X(293)- is selected from the group consisting of E and R;
—X(295)- is selected from the group consisting of Q and E;
—X(296)- is selected from the group consisting of F and Y;
—X(300)- is selected from the group consisting of F and Y;
—X(304)- is selected from the group consisting of S and T;
—X(309)- is selected from the group consisting of V and L;
—X(324)- is selected from the group consisting of S, G and I;
—X(326)- is selected from the group consisting of K and T;
—X(327)- is selected from the group consisting of G, A and D;
—X(328)- is selected from the group consisting of L, A, F, I and T;
—X(330)- is selected from the group consisting of A, S, L, Y and I;
—X(331)- is selected from the group consisting of P and S;
—X(332)- is selected from the group consisting of I, D, E, N, Q and T;
—X(333)- is selected from the group consisting of E and Y;
—X(334)- is selected from the group consisting of K, F, I and T;
—X(339)- is selected from the group consisting of T and A;
—X(355)- is selected from the group consisting of R and Q;
—X(356)- is selected from the group consisting of E and D;
—X(358)- is selected from the group consisting of M and L;
—X(384)- is selected from the group consisting of N and S;
—X(392)- is selected from the group consisting of K and N;
—X(397)- is selected from the group consisting of M and V;
—X(409)- is selected from the group consisting of K and R;
—X(419)- is selected from the group consisting of Q and E;
—X(422)- is selected from the group consisting of V and I;
—X(435)- is selected from the group consisting of H and R;
—X(436)- is selected from the group consisting of Y and F; and
—X(445)- is selected from the group consisting of P and L.

In certain variations, a first modification is selected from among C131S, R133K, G137E, G138S, S192N, L193F, Q196K, T199I, N203D, R214K R214T, L217P, L217R, L217S, T219S, T219C, T219Y, P220C P220G, L221D, deletion of L221, deletion of the sequence LGD beginning at L221, T222K, T222V, deletion of T222, deletion of T223, H224E, H224P, deletion of T225, T225P, R228P, R228S, deletion of R, deletion of the sequence RCPEPKSCDTPPPCPRCPEPKSCDTPPPCPRCPEPKSCDTPPPCPR (SEQ ID NO: 111) beginning at 228, E233P, L234V, L234F, L235A, deletion of G236, H268Q, Q274K, K276N, Y296F, F300Y, L309V, A327G, A330S, P331S, T339A, R355Q, E356D, M358L, 5384N, N392K, M397V, K409R, Q419E, I422V, R435H, F436Y, and P445L. In a further variation, a second modification is selected from among 221K, 227G, 234Y, 234I, 235Y, 235I, 235D, 236S, 236A, 237D, 239D, 239E, 239N, 239Q, 239T, 240I, 240M, 246H, 246Y, 255Y, 258H, 258Y, 260H, 264I, 264T, 264Y, 267D, 267E, 268D, 268E, 271G, 272Y, 272H, 272R, 272I, 274E, 278T, 281D, 281E, 283L, 283H, 284E, 284D, 290N, 293R, 295E, 304T, 324G, 324I, 326T, 327D, 328A, 328F, 328I, 328T, 330L, 330Y, 330I, 332D, 332E, 332N, 332Q, 332T, 333Y, 334F, 334I, and 334T. In various embodiments, the formula has at least 1, 2, 3, 4, 5, 6, 7, 8, 9, 10 or more first and/or second amino acid modifications as compared to an amino acid sequence including SEQ ID NO:12. In additional embodiments, at least 2, 3, or 4 of the modifications are in different domains.

In another aspect, the present application is directed to an IgG3 variant including an amino acid sequence having the formula:

(SED ID NO: 100)
C-X(227)-X(228)-C-X(230)-X(231)-X(232)-X(233)-X(234)-X(235)-X(236)-X(237)-X(238)-
X(239)-X(240)-X(241)-L-X(243)-X(244)-X(245)-X(246)-X(247)-K-X(249)-TLMIS-X(255)-
TP-X(258)-V-X(260)-C-X(262)-X(263)-X(264)-X(265)-X(266)-X(267)-X(268)-X(269)-
X(270)-X(271)-X(272)-X(273)-X(274)-X(275)-X(276)-W-X(278)-V-X(280)-X(281)-
X(282)-X(283)-X(284)-X(285)-X(286)-A-X(288)-T-X(290)-X(291)-X(292)-X(293)-X(294)-
X(295)-X(296)-X(297)-X(298)-X(299)-X(300)-X(301)-X(302)-X(303)-X(304)-X(305)-
LTV-X(309)-HQD-X(313)-LNG-X(317)-X(318)-Y-X(320)-C-X(322)-X(323)-X(324)-
X(325)-X(326)-X(327)-X(328)-X(329)-X(330)-X(331)-X(332)-X(333)-X(334)-X(335)-
X(336)-X(337)-K-X(339)-KGQPREPQVYTLPPS-X(355)-X(356)-E-X(358)-
TKNQVSLTCLVKGFYPSDIAVEWES-X(384)-GQPENNY-X(392)-TTPP-X(397)-
LDSDGSFFLYS-X(409)-LTVDKSRWQ-X(419)-GN-X(422)-FSCSVMHEALHN-X(435)-
X(436)-TQKSLSLS-X(445)-GK,

wherein
—X(227)- is selected from the group consisting of P, E, G, K and Y;
—X(228)- is selected from the group consisting of P, S, E, G, K, Y, R, and the sequence

(SEQ ID NO: 111)

RCPEPKSCDTPPPCPRCPEPKSCDTPPPCPRCPEPKSCDTPPPCPR;

—X(230)- is selected from the group consisting of P, A, E, G and Y;
—X(231)- is selected from the group consisting of A, E, G, K, P and Y;
—X(232)- is selected from the group consisting of P, E, G, K and Y;
—X(233)- is selected from the group consisting of P, E, A, D, F, G, H, I, K, L, M, N, Q, R, S, T, V, W and Y;
—X(234)- is selected from the group consisting of V, L, F, D, E, F, G, H, I, K, M, N, P, Q, R, S, T, W and Y;
—X(235)- is selected from the group consisting of A, L, D, F, G, H, I, K, M, N, P, Q, R, S, T, V, W, and Y;
—X(236)- is selected from the group consisting of no amino acid, G, A, D, E, F, H, I, K, L, M, N, P, Q, R, S, T, V, W and Y;
—X(237)- is selected from the group consisting of G, D, E, F, H, I, K, L, M, N, P, Q, R, S, T, V, W and Y;
—X(238)- is selected from the group consisting of P, D, E, F, G, H, I, K, L, M, N, Q, R, S, T, V, W and Y;
—X(239)- is selected from the group consisting of S, D, E, F, G, H, I, K, L, M, N, P, Q, R, T, V, W and Y;
—X(240)- is selected from the group consisting of V, A, I, M and T;
—X(241)- is selected from the group consisting of F, D, E, L, R, S, W and Y;
—X(243)- is selected from the group consisting of F, E, H, L, Q, R, W, and Y;
—X(244)- is selected from the group consisting of P and H;
—X(245)- is selected from the group consisting of P and A;
—X(246)- is selected from the group consisting of, K, D, E, H and Y;
—X(247)- is selected from the group consisting of P, G and V;
—X(249)- is selected from the group consisting of D, H, Q and Y;
—X(255)- is selected from the group consisting of RE and Y;
—X(258)- is selected from the group consisting of E, H, S and Y;
—X(260)- is selected from the group consisting of T, D, E, H and Y;
—X(262)- is selected from the group consisting of V, A, E, F, I and T;
—X(263)- is selected from the group consisting of V, A, I, M and T;
—X(264)- is selected from the group consisting of V, A, D, E, F, G, H, I, K, L, M, N, P, Q, R, S, T, W and Y;
—X(265)- is selected from the group consisting of D, F, G, H, I, K, L, M, P, Q, R, S, T, V, W and Y;
—X(266)- is selected from the group consisting of V, A, I, M and T;
—X(267)- is selected from the group consisting of S, D, E, F, H, I, K, L, M, N, P, Q, R, V, W and Y;
—X(268)- is selected from the group consisting of H, Q, D, E, F, G, I, K, L, M, P, R, T, V and W;
—X(269)- is selected from the group consisting of E, F, G, H, I, K, L, M, N, P, R, S, T, V, W and Y;
—X(270)- is selected from the group consisting of D, F, G, H, I, L, M, P, Q, R, S, T, W and Y;
—X(271)- is selected from the group consisting of P, A, D, E, F, G, H, I, K, L, M, N, Q, R, S, T, V, W and Y;
—X(272)- is selected from the group consisting of E, D, F, G, H, I, K, L, M, P, R, S, T, V, W and Y;
—X(273)- is selected from the group consisting of V and I;
—X(274)- is selected from the group consisting of Q, K, D, E, F, G, H, I, L, M, N, P, R, T, V, W and Y;
—X(275)- is selected from the group consisting of FL and W;
—X(276)- is selected from the group consisting of N, K, D, E, F, G, H, I, L, M, P, R, S, T, V, W and Y;
—X(278)- is selected from the group consisting of Y, D, E, G, H, I, K, L, M, N, P, Q, R, S, T, V and W;
—X(280)- is selected from the group consisting of D, G, K, L, P and W;
—X(281)- is selected from the group consisting of G, D, E, K, N, P, Q and Y;
—X(282)- is selected from the group consisting of V, E, G, K, P and Y;
—X(283)- is selected from the group consisting of E, G, H, K, L, P, R and Y;
—X(284)- is selected from the group consisting of V, D, E, L, N, Q, T and Y;
—X(285)- is selected from the group consisting of H, D, E, K, Q, W and Y;
—X(286)- is selected from the group consisting of N, E, G, P and Y;
—X(288)- is selected from the group consisting of K, D, E and Y;
—X(290)- is selected from the group consisting of K, D, H, L, N and W;
—X(291)- is selected from the group consisting of P, D, E, G, H, I, Q and T;
—X(292)- is selected from the group consisting of R, D, E, T and Y;
—X(293)- is selected from the group consisting of E, F, G, H, I, L, M, N, P, R, S, T, V, W and Y;
—X(294)- is selected from the group consisting of E, F, G, H, I, K, L, M, P, R, S, T, V, W and Y;
—X(295)- is selected from the group consisting of Q, D, E, F, G, H, I, M, N, P, R, S, T, V, W and Y;
—X(296)- is selected from the group consisting of F, Y, A, D, E, G, I, K, L, M, N, Q, R, S, T and V;
—X(297)- is selected from the group consisting of N, D, E, F, G, H, I, K, L, M, P, Q, R, S, T, V, W and Y;
—X(298)- is selected from the group consisting of S, E, F, H, I, K, M, Q, R, W and Y;
—X(299)- is selected from the group consisting of T, A, D, E, F, G, H, I, K, L, M, N, P, Q, R, S, V, W and Y;
—X(300)- is selected from the group consisting of F, Y, A, D, E, G, H, K, M, N, P, Q, R, S, T, V and W;
—X(301)- is selected from the group consisting of R, D, E, H and Y;
—X(302)- is selected from the group consisting of V and I;
—X(303)- is selected from the group consisting of V, D, E and Y;
—X(304)- is selected from the group consisting of S, D, H, L, N and T;
—X(305)- is selected from the group consisting of V, E, T and Y;
—X(309)- is selected from the group consisting of V and L;
—X(313)- is selected from the group consisting of W and F;
—X(317)- is selected from the group consisting of K, E and Q;
—X(318)- is selected from the group consisting of E, H, L, Q, R and Y;
—X(320)- is selected from the group consisting of K, D, F, G, H, I, L, N, P, S, T, V, W and Y;
—X(322)- is selected from the group consisting of K, D, F, G, H, I, P, S, T, V, W and Y;
—X(323)- is selected from the group consisting of V and I;
—X(324)- is selected from the group consisting of S, D, F, G, H, I, L, M, P, R, T, V, W and Y;
—X(325)- is selected from the group consisting of N, A, D, E, F, G, H, I, K, L, M, P, Q, R, S, T, V, W and Y;
—X(326)- is selected from the group consisting of K, I, L, P and T;
—X(327)- is selected from the group consisting of G, A, D, E, F, H, I, K, L, M, N, P, R, T, V, W and Y;
—X(328)- is selected from the group consisting of L, A, D, E, F, G, H, I, K, M, N, P, Q, R, S, T, V, W and Y;
—X(329)- is selected from the group consisting of P, D, E, F, G, H, I, K, L, M, N, Q, R, S, T, V, W and Y;
—X(330)- is selected from the group consisting of A, S, E, F, G, H, I, L, M, N, P, R, T, V, W and Y;
—X(331)- is selected from the group consisting of P, S, D, F, H, I, L, M, Q, R, T, V, W and Y;
—X(332)- is selected from the group consisting of I, A, D, E, F, H, K, L, M, N, P, Q, R, S, T, V, W and Y;
—X(333)- is selected from the group consisting of E, F, H, I, L, M, P, T and Y;
—X(334)- is selected from the group consisting of K, F, I, P and T;
—X(335)- is selected from the group consisting of T, D, F, G, H, I, L, M, N, P, R, S, V, W and Y;
—X(336)- is selected from the group consisting of I, E, K and Y;
—X(337)- is selected from the group consisting of S, E, H and N;
—X(339)- is selected from the group consisting of T and A;
—X(355)- is selected from the group consisting of R and Q;
—X(356)- is selected from the group consisting of E and D;
—X(358)- is selected from the group consisting of M and L;
—X(384)- is selected from the group consisting of N and S;
—X(392)- is selected from the group consisting of K and N;
—X(397)- is selected from the group consisting of M and V;
—X(409)- is selected from the group consisting of K and R;
—X(419)- is selected from the group consisting of Q and E;
—X(422)- is selected from the group consisting of V and I;
—X(435)- is selected from the group consisting of H and R;
—X(436)- is selected from the group consisting of Y and F; and
—X(445)- is selected from the group consisting of P and L

In various embodiments, a first modification is selected from among R228P, R228S, deletion of the sequence

(SEQ ID NO: 111)

RCPEPKSCDTPPPCPRCPEPKSCDTPPPCPRCPEPKSCDTPPPCPR

beginning at 228, E233P, L234V, L234F, L235A, deletion of G236, H268Q, Q274K, K276N, Y296F, F300Y, L309V, A327G, A330S, P331S, T339A, R355Q, E356D, M358L, S384N, N392K, M397V, K409R, Q419E, I422V, R435H, F436Y, and P445L, and/or a second modification is selected from among 237D, 237E, 237F, 237H, 237I, 237K, 237L, 237M, 237N, 237P, 237Q, 237R, 237S, 237T, 237V, 237W, 237Y, 238D, 238E, 238F, 238G, 238H, 238I, 238K, 238L, 238M, 238N, 238Q, 238R, 238S, 238T, 238V, 238W, 238Y, 239D, 239E, 239F, 239G, 239H, 239I, 239K, 239L, 239M, 239N, 239P, 239Q, 239R, 239T, 239V, 239W, 239Y, 240A, 240I, 240M, 240T, 241D, 241E, 241L, 241R, 241S, 241W, 241Y, 243E, 243H, 243L, 243Q, 243R, 243W, 243Y, 244H, 245A, 246D, 246E, 246H, 246Y, 247G, 247V, 249H, 249Q, 249Y, 255E, 255Y, 258H, 258S, 258Y, 260D, 260E, 260H, 260Y, 262A, 262E, 262F, 262I, 262T, 263A, 263I, 263M, 263T, 264A, 264D, 264E, 264F, 264G, 264H, 264I, 264K, 264L, 264M, 264N, 264P, 264Q, 264R, 264S, 264T, 264W, 264Y, 265F, 265G, 265H, 265I, 265K, 265L, 265M, 265P, 265Q, 265R, 265S, 265T, 265V, 265W, 265Y, 266A, 266I, 266M, 266T, 267D, 267E, 267F, 267H, 267I, 267K, 267L, 267M, 267N, 267P, 267Q, 267R, 267V, 267W, 267Y, 268D, 268E, 268F, 268G, 268I, 268K, 268L, 268M, 268P, 268R, 268T, 268V, 268W, 269F, 269G, 269H, 269I, 269K, 269L, 269M, 269N, 269P, 269R, 269S, 269T, 269V, 269W, 269Y, 270F, 270G, 270H, 270I, 270L, 270M, 270P, 270Q, 270R, 270S, 270T, 270W, 270Y, 271A, 271D, 271E, 271F, 271G, 271H, 271I, 271K, 271L, 271M, 271N, 271Q, 271R, 271S, 271T, 271V, 271W, 271Y, 272D, 272F, 272G, 272H, 272I, 272K, 272L, 272M, 272P, 272R, 272S, 272T, 272V, 272W, 272Y, 273I, 274D, 274E, 274F, 274G, 274H, 274I, 274L, 274M, 274N, 274P, 274R, 274T, 274V, 274W, 274Y, 275L, 275W, 276D, 276E, 276F, 276G, 276H, 276I, 276L, 276M, 276P, 276R, 276S, 276T, 276V, 276W, 276Y, 278D, 278E, 278G, 278H, 278I, 278K, 278L, 278M, 278N, 278P, 278Q, 278R, 278S, 278T, 278V, 278W, 280G, 280K, 280L, 280P, 280W, 281D, 281E, 281K, 281N, 281P, 281Q, 281Y, 282E, 282G, 282K, 282P, 282Y, 283G, 283H, 283K, 283L, 283P, 283R, 283Y, 284D, 284E, 284L, 284N, 284Q, 284T, 284Y, 285D, 285E, 285K, 285Q, 285W, 285Y, 286E, 286G, 286P, 286Y, 288D, 288E, 288Y, 290D, 290H, 290L, 290N, 290W, 291D, 291E, 291G, 291H, 291I, 291Q, 291T, 292D, 292E, 292T, 292Y, 293F, 293G, 293H, 293I, 293L, 293M, 293N, 293P, 293R, 293S, 293T, 293V, 293W, 293Y, 294F, 294G, 294H, 294I, 294K, 294L, 294M, 294P, 294R, 294S, 294T, 294V, 294W, 294Y, 295D, 295E, 295F, 295G, 295H, 295I, 295M, 295N, 295P, 295R, 295S, 295T, 295V, 295W, 295Y, 296A, 296D, 296E, 296G, 296I, 296K, 296L, 296M, 296N, 296Q, 296R, 296S, 296T, 296V, 297D, 297E, 297F, 297G, 297H, 297I, 297K, 297L, 297M, 297P, 297Q, 297R, 297S, 297T, 297V, 297W, 297Y, 298E, 298F, 298H, 298I, 298K, 298M, 298Q, 298R, 298W, 298Y, 299A, 299D, 299E, 299F, 299G, 299H, 299I, 299K, 299L, 299M, 299N, 299P, 299Q, 299R, 299S, 299V, 299W, 299Y, 300A, 300D, 300E, 300G, 300H, 300K, 300M, 300N, 300P, 300Q, 300R, 300S, 300T, 300V, 300W, 301D, 301E, 301H, 301Y, 302I, 303D, 303E, 303Y, 304D, 304H, 304L, 304N, 304T, 305E, 305T, 305Y, 313F, 317E, 317Q, 318H, 318L, 318Q, 318R, 318Y, 320D, 320F, 320G, 320H, 320I, 320L, 320N, 320P, 320S, 320T, 320V, 320W, 320Y, 322D, 322F, 322G, 322H, 322I, 322P, 322S, 322T, 322V, 322W, 322Y, 323I, 324D, 324F, 324G, 324H, 324I, 324L, 324M, 324P, 324R, 324T, 324V, 324W, 324Y, 325A, 325D, 325E, 325F, 325G, 325H, 325I, 325K, 325L, 325M, 325P, 325Q, 325R, 325S, 325T, 325V, 325W, 325Y, 326I, 326L, 326P, 326T, 327D, 327E, 327F, 327H, 327I, 327K, 327L, 327M, 327N, 327P, 327R, 327T, 327V, 327W, 327Y, 328A, 328D, 328E, 328F, 328G, 328H, 328I, 328K, 328M, 328N, 328P, 328Q, 328R, 328S, 328T, 328V, 328W, 328Y, 329D, 329E, 329F, 329G, 329H, 329I, 329K, 329L, 329M, 329N, 329Q, 329R, 329S, 329T, 329V, 329W, 329Y, 330E, 330F, 330G, 330H, 330I, 330L, 330M, 330N, 330P, 330R, 330T, 330V, 330W, 330Y, 331D, 331F, 331H, 331I, 331L, 331M, 331Q, 331R, 331T, 331V, 331W, 331Y, 332A, 332D, 332E, 332F, 332H, 332K, 332L, 332M, 332N, 332P, 332Q, 332R, 332S, 332T, 332V, 332W, 332Y, 333F, 333H, 333I, 333L, 333M, 333P, 333T, 333Y, 334F, 334I, 334P, 334T, 335D, 335F, 335G, 335H, 335I, 335L, 335M, 335N, 335P, 335R, 335S, 335V, 335W, 335Y, 336E, 336K, 336Y, 337E, 337H, and 337N. In various embodiments, the formula has at least 1, 2, 3, 4, 5, 6, 7, 8, 9, 10 or more amino acid modifications as compared to an amino acid sequence including SEQ ID NO:12. In additional embodiments, at least 2, 3, or 4 of the first and/or second modifications are in different domains. Alternatively, the substitutions can be selected from those beginning at position 230.

In another aspect, the present application is directed to an IgG3 variant amino acid sequence including at least two modifications as compared to SEQ ID NO:12, wherein a first modification is selected from among R228P, R228S, deletion of the sequence

(SEQ ID NO: 111)

RCPEPKSCDTPPPCPRCPEPKSCDTPPPCPRCPEPKSCDTPPPCPR

beginning at 228, E233P, L234V, L234F, L235A, deletion of G236, H268Q, Q274K, K276N, Y296F, F300Y, L309V, A327G, A330S, P331S, T339A, R355Q, E356D, M358L, 5384N, N392K, M397V, K409R, Q419E, I422V, R435H, F436Y, and P445L. In a further variation, a second modification is selected from among 227E, 227G, 227K, 227Y, 228E, 228G, 228K, 228Y, 230A, 230E, 230G, 230Y, 231E, 231G, 231K, 231P, 231Y, 232E, 232G, 232K, 232Y, 233A, 233D, 233F, 233G, 233H, 233I, 233K, 233L, 233M, 233N, 233Q, 233R, 233S, 233T, 233V, 233W, 233Y, 234D, 234E, 234F, 234G, 234H, 234I, 234K, 234M, 234N, 234P, 234Q, 234R, 234S, 234T, 234W, 234Y, 235D, 235F, 235G, 235H, 235I, 235K, 235M, 235N, 235P, 235Q, 235R, 235S, 235T, 235V, 235W, 235Y, 236A, 236D, 236E, 236F, 236H, 236I, 236K, 236L, 236M, 236N, 236P, 236Q, 236R, 236S, 236T, 236V, 236W, 236Y, 237D, 237E, 237F, 237H, 237I, 237K, 237L, 237M, 237N, 237P, 237Q, 237R, 237S, 237T, 237V, 237W, 237Y, 238D, 238E, 238F, 238G, 238H, 238I, 238K, 238L, 238M, 238N, 238Q, 238R, 238S, 238T, 238V, 238W, 238Y, 239D, 239E, 239F, 239G, 239H, 239I, 239K, 239L, 239M, 239N, 239P, 239Q, 239R, 239T, 239V, 239W, 239Y, 240A, 240I, 240M, 240T, 241D, 241E, 241L, 241R, 241S, 241W, 241Y, 243E, 243H, 243L, 243Q, 243R, 243W, 243Y, 244H, 245A, 246D, 246E, 246H, 246Y, 247G, 247V, 249H, 249Q, 249Y, 255E, 255Y, 258H, 258S, 258Y, 260D, 260E, 260H, 260Y, 262A, 262E, 262F, 262I, 262T, 263A, 263I, 263M, 263T, 264A, 264D, 264E, 264F, 264G, 264H, 264I, 264K, 264L, 264M, 264N, 264P, 264Q, 264R, 264S, 264T, 264W, 264Y, 265F, 265G, 265H, 265I, 265K, 265L, 265M, 265P, 265Q, 265R, 265S, 265T, 265V, 265W, 265Y, 266A, 266I, 266M, 266T, 267D, 267E, 267F, 267H, 267I, 267K, 267L, 267M, 267N, 267P, 267Q, 267R, 267V, 267W, 267Y, 268D, 268E, 268F, 268G, 268I, 268K, 268L, 268M, 268P, 268R, 268T, 268V, 268W, 269F, 269G, 269H, 269I, 269K, 269L, 269M, 269N, 269P, 269R, 269S, 269T, 269V, 269W, 269Y, 270F, 270G, 270H, 270I, 270L, 270M, 270P, 270Q, 270R, 270S, 270T, 270W, 270Y, 271A, 271D, 271E, 271F, 271G, 271H, 271I, 271K, 271L, 271M, 271N, 271Q, 271R, 271S, 271T, 271V, 271W, 271Y, 272D, 272F, 272G, 272H, 272I, 272K, 272L, 272M, 272P, 272R, 272S, 272T, 272V, 272W, 272Y, 273I, 274D, 274E, 274F, 274G, 274H, 274I, 274L, 274M, 274N, 274P, 274R, 274T, 274V, 274W, 274Y, 275L, 275W, 276D, 276E, 276F, 276G, 276H, 276I, 276L, 276M, 276P, 276R, 276S, 276T, 276V, 276W, 276Y, 278D, 278E, 278G, 278H, 278I, 278K, 278L, 278M, 278N, 278P, 278Q, 278R, 278S, 278T, 278V, 278W, 280G, 280K, 280L, 280P, 280W, 281D, 281E, 281K, 281N, 281P, 281Q, 281Y, 282E, 282G, 282K, 282P, 282Y, 283G, 283H, 283K, 283L, 283P, 283R, 283Y, 284D, 284E, 284L, 284N, 284Q, 284T, 284Y, 285D, 285E, 285K, 285Q, 285W, 285Y, 286E, 286G, 286P, 286Y, 288D, 288E, 288Y, 290D, 290H, 290L, 290N, 290W, 291D, 291E, 291G, 291H, 291I, 291Q, 291T, 292D, 292E, 292T, 292Y, 293F, 293G, 293H, 293I, 293L, 293M, 293N, 293P, 293R, 293S, 293T, 293V, 293W, 293Y, 294F, 294G, 294H, 294I, 294K, 294L, 294M, 294P, 294R, 294S, 294T, 294V, 294W, 294Y, 295D, 295E, 295F, 295G, 295H, 295I, 295M, 295N, 295P, 295R, 295S, 295T, 295V, 295W, 295Y, 296A, 296D, 296E, 296G, 296I, 296K, 296L, 296M, 296N, 296Q, 296R, 296S, 296T, 296V, 297D, 297E, 297F, 297G, 297H, 297I, 297K, 297L, 297M, 297P, 297Q, 297R, 297S, 297T, 297V, 297W, 297Y, 298E, 298F, 298H, 298I, 298K, 298M, 298Q, 298R, 298W, 298Y, 299A, 299D, 299E, 299F, 299G, 299H, 299I, 299K, 299L, 299M, 299N, 299P, 299Q, 299R, 299S, 299V, 299W, 299Y, 300A, 300D, 300E, 300G, 300H, 300K, 300M, 300N, 300P, 300Q, 300R, 300S, 300T, 300V, 300W, 301D, 301E, 301H, 301Y, 302I, 303D, 303E, 303Y, 304D, 304H, 304L, 304N, 304T, 305E, 305T, 305Y, 313F, 317E, 317Q, 318H, 318L, 318Q, 318R, 318Y, 320D, 320F, 320G, 320H, 320I, 320L, 320N, 320P, 320S, 320T, 320V, 320W, 320Y, 322D, 322F, 322G, 322H, 322I, 322P, 322S, 322T, 322V, 322W, 322Y, 323I, 324D, 324F, 324G, 324H, 324I, 324L, 324M, 324P, 324R, 324T, 324V, 324W, 324Y, 325A, 325D, 325E, 325F, 325G, 325H, 325I, 325K, 325L, 325M, 325P, 325Q, 325R, 325S, 325T, 325V, 325W, 325Y, 326I, 326L, 326P, 326T, 327D, 327E, 327F, 327H, 327I, 327K, 327L, 327M, 327N, 327P, 327R, 327T, 327V, 327W, 327Y, 328A, 328D, 328E, 328F, 328G, 328H, 328I, 328K, 328M, 328N, 328P, 328Q, 328R, 328S, 328T, 328V, 328W, 328Y, 329D, 329E, 329F, 329G, 329H, 329I, 329K, 329L, 329M, 329N, 329Q, 329R, 329S, 329T, 329V, 329W, 329Y, 330E, 330F, 330G, 330H, 330I, 330L, 330M, 330N, 330P, 330R, 330T, 330V, 330W, 330Y, 331D, 331F, 331H, 331I, 331L, 331M, 331Q, 331R, 331T, 331V, 331W, 331Y, 332A, 332D, 332E, 332F, 332H, 332K, 332L, 332M, 332N, 332P, 332Q, 332R, 332S, 332T, 332V, 332W, 332Y, 333F, 333H, 333I, 333L, 333M, 333P, 333T, 333Y, 334F, 334I, 334P, 334T, 335D, 335F, 335G, 335H, 335I, 335L, 335M, 335N, 335P, 335R, 335S, 335V, 335W, 335Y, 336E, 336K, 336Y, 337E, 337H, and 337N. In various embodiments, the formula has at least 1, 2, 3, 4, 5, 6, 7, 8, 9, 10 or more amino acid modifications as compared to an amino acid sequence including SEQ ID NO:12. In additional embodiments, at least 2, 3, or 4 of the modifications are in different domains.

In another aspect, the present application is directed to an IgG3 variant including an amino acid sequence having the formula:

(SED ID NO: 101)
C-X(227)-X(228)-CPAP-
X(233)-X(234)-X(235)-X(236)-X(237)-P-X(239)-X(240)-FLFPP-X(246)-PKDTLMIS-
X(255)-TP-X(258)-V-X(260)-CVV-X(264)-DV-X(267)-X(268)-ED-X(271)-X(272)-V-
X(274)-F-X(276)-W-X(278)-VD-X(281)-V-X(283)-X(284)-HNAKT-X(290)-PR-X(293)-E-
X(295)-X(296)-NST-X(300)-RVV-X(304)-VLTV-X(309)-HQDWLNGKEYKCKV-X(324)-
N-X(326)-X(327)-X(328)-P-X(330)-X(331)-X(332)-X(333)-X(334)-TISK-X(339)-
KGQPREPQVYTLPPS-X(355)-X(356)-E-X(358)-TKNQVSLTCLVKGFYPSDIAVEWES-
X(384)-GQPENNY-X(392)-TTPP-X(397)-LDSDGSFFLYS-X(409)-LTVDKSRWQ-
X(419)-GN-X(422)-FSCSVMHEALHN-X(435)-X(436)-TQKSLSLS-X(445)-GK-,

wherein
—X(227)- is selected from the group consisting of P and G;
—X(228)- is selected from the group consisting of P, R, S, and the sequence

(SEQ ID NO: 111)

RCPEPKSCDTPPPCPRCPEPKSCDTPPPCPRCPEPKSCDTPPPCPR;

—X(233)- is selected from the group consisting of P and E;
—X(234)- is selected from the group consisting of V, L, F, Y and I;
—X(235)- is selected from the group consisting of A, L, Y, I and D;
—X(236)- is selected from the group consisting of no amino acid, G, S and A;
—X(237)- is selected from the group consisting of G and D;
—X(239)- is selected from the group consisting of S, D, E, N, Q and T;
—X(240)- is selected from the group consisting of V, I and M;
—X(246)- is selected from the group consisting of K, H and Y;
—X(255)- is selected from the group consisting of R and Y;
—X(258)- is selected from the group consisting of E, H and Y;
—X(260)- is selected from the group consisting of T and H;
—X(264)- is selected from the group consisting of V, I, T and Y;
—X(267)- is selected from the group consisting of S, D and E;
—X(268)- is selected from the group consisting of H, Q, D and E;
—X(271)- is selected from the group consisting of P and G;
—X(272)- is selected from the group consisting of E, Y, H, R and I;
—X(274)- is selected from the group consisting of Q, K and E;
—X(276)- is selected from the group consisting of N and K;
—X(278)- is selected from the group consisting of Y and T;
—X(281)- is selected from the group consisting of G, D and E;
—X(283)- is selected from the group consisting of E, L and H;
—X(284)- is selected from the group consisting of V, E and D;
—X(290)- is selected from the group consisting of K and N;
—X(293)- is selected from the group consisting of E and R;
—X(295)- is selected from the group consisting of Q and E;
—X(296)- is selected from the group consisting of F and Y;
—X(300)- is selected from the group consisting of F and Y;
—X(304)- is selected from the group consisting of S and T;
—X(309)- is selected from the group consisting of V and L;
—X(324)- is selected from the group consisting of S, G and I;
—X(326)- is selected from the group consisting of K and T;
—X(327)- is selected from the group consisting of G, A and D;
—X(328)- is selected from the group consisting of L, A, F, I and T;
—X(330)- is selected from the group consisting of A, S, L, Y and I;
—X(331)- is selected from the group consisting of P and S;
—X(332)- is selected from the group consisting of I, D, E, N, Q and T;
—X(333)- is selected from the group consisting of E and Y;
—X(334)- is selected from the group consisting of K, F, I and T;
—X(339)- is selected from the group consisting of T and A;
—X(355)- is selected from the group consisting of R and Q;
—X(356)- is selected from the group consisting of E and D;
—X(358)- is selected from the group consisting of M and L;
—X(384)- is selected from the group consisting of N and S;
—X(392)- is selected from the group consisting of K and N;
—X(397)- is selected from the group consisting of M and V;
—X(409)- is selected from the group consisting of K and R;
—X(419)- is selected from the group consisting of Q and E;
—X(422)- is selected from the group consisting of V and I;
—X(435)- is selected from the group consisting of H and R;
—X(436)- is selected from the group consisting of Y and F; and
—X(445)- is selected from the group consisting of P and L.

In certain variations, a first modification is selected from among R228P, R228S, deletion of R, deletion of the sequence RCPEPKSCDTPPPCPRCPEPKSCDTPPPCPRCPEPKSCDTPPPCPR (SEQ ID NO: 111) beginning at 228, E233P, L234V, L234F, L235A, deletion of G236, H268Q, Q274K, K276N, Y296F, F300Y, L309V, A327G, A330S, P331S, T339A, R355Q, E356D, M358L, 5384N, N392K, M397V, K409R, Q419E, I422V, R435H, F436Y, and P445L. In further variations, a second modification is selected from among 227G, 234Y, 234I, 235Y, 235I, 235D, 236S, 236A, 237D, 239D, 239E, 239N, 239Q, 239T, 240I, 240M, 246H, 246Y, 255Y, 258H, 258Y, 260H, 264I, 264T, 264Y, 267D, 267E, 268D, 268E, 271G, 272Y, 272H, 272R, 272I, 274E, 278T, 281D, 281E, 283L, 283H, 284E, 284D, 290N, 293R, 295E, 304T, 324G, 324I, 326T, 327D, 328A, 328F, 328I, 328T, 330L, 330Y, 330I, 332D, 332E, 332N, 332Q, 332T, 333Y, 334F, 334I, and 334T. In additional embodiments, the formula has at least 1, 2, 3, 4, 5, 6, 7, 8, 9, 10 or more amino acid modifications as compared to an amino acid sequence including SEQ ID NO:12. In additional embodiments, at least 2, 3, or 4 of the modifications are in different domains. Alternatively, the modifications can be from position 230 until the C terminus.

In another aspect, the present application is directed to an IgG3 variant including an amino acid sequence having the formula:

(SED ID NO: 102)
ASTKGPSVFPLAPCSRSTSGGTAALGCLVKDYFPEPVTVSWNSGALTSGVHTFPAVL
QSSGLYSLSSVVTVPSSSLGTQTYTCNVNHKPSNTKVDKRVELKTP-X(221)-GD-
X(222)-X(223)-X(224)-X(225)-C-X(227)-X(228)-
CPEPKSCDTPPPCPRCPEPKSCDTPPPCPRCPEPKSCDTPPPCPRC-X(230)-X(231)-
X(232)-X(233)-X(234)-X(235)-X(236)-X(237)-X(238)-X(239)-X(240)-X(241)-L-X(243)-
X(244)-X(245)-X(246)-X(247)-K-X(249)-TLMIS-X(255)-TP-X(258)-V-X(260)-C-X(262)-
X(263)-X(264)-X(265)-X(266)-X(267)-X(268)-X(269)-X(270)-X(271)-X(272)-X(273)-
X(274)-X(275)-X(276)-W-X(278)-V-X(280)-X(281)-X(282)-X(283)-X(284)-X(285)-
X(286)-A-X(288)-T-X(290)-X(291)-X(292)-X(293)-X(294)-X(295)-X(296)-X(297)-X(298)-
X(299)-X(300)-X(301)-X(302)-X(303)-X(304)-X(305)-LTVLHQD-X(313)-LNG-X(317)-
X(318)-Y-X(320)-C-X(322)-X(323)-X(324)-X(325)-X(326)-X(327)-X(328)-X(329)-X(330)-
X(331)-X(332)-X(333)-X(334)-X(335)-X(336)-X(337)-KTKGQPREPQVYTLPPSREEMTK
NQVSLTCLVKGFYPSDIAVEWESSGQPENNYNTTPPMLDSDGSFFLYSKLTVD
KSRWQQGNIFSCSVMHEALHNRFTQKSLSLSPGK,

wherein
—X(221)- is selected from the group consisting of L, K and Y;
—X(222)- is selected from the group consisting of T, E and Y;
—X(223)- is selected from the group consisting of T, E and K;
—X(224)- is selected from the group consisting of H and Y;
—X(225)- is selected from the group consisting of T, E, K and W;
—X(227)- is selected from the group consisting of P, E, G, K and Y;
—X(228)- is selected from the group consisting of R, E, G, K and Y;
—X(230)- is selected from the group consisting of P, A, E, G and Y;
—X(231)- is selected from the group consisting of A, E, G, K, P and Y;
—X(232)- is selected from the group consisting of P, E, G, K and Y;
—X(233)- is selected from the group consisting of E, A, D, F, G, H, I, K, L, M, N, Q, R, S, T, V, W and Y;
—X(234)- is selected from the group consisting of L, D, E, F, G, H, I, K, M, N, P, Q, R, S, T, W and Y;
—X(235)- is selected from the group consisting of L, D, F, G, H, I, K, M, N, P, Q, R, S, T, V, W, and Y;
—X(236)- is selected from the group consisting of G, A, D, E, F, H, I, K, L, M, N, P, Q, R, S, T, V, W and Y;
—X(237)- is selected from the group consisting of G, D, E, F, H, I, K, L, M, N, P, Q, R, S, T, V, W, and Y;
—X(238)- is selected from the group consisting of P, D, E, F, G, H, I, K, L, M, N, Q, R, S, T, V, W and Y;
—X(239)- is selected from the group consisting of S, D, E, F, G, H, I, K, L, M, N, P, Q, R, T, V, W and Y;
—X(240)- is selected from the group consisting of V, A, I, M and T;
—X(241)- is selected from the group consisting of F, D, E, L, R, S, W and Y;
—X(243)- is selected from the group consisting of F, E, H, L, Q, R, W and Y;
—X(244)- is selected from the group consisting of P and H;
—X(245)- is selected from the group consisting of P and A;
—X(246)- is selected from the group consisting of K, D, E, H and Y;
—X(247)- is selected from the group consisting of P, G and V;
—X(249)- is selected from the group consisting of D, H, Q and Y;
—X(255)- is selected from the group consisting of R, E and Y;
—X(258)- is selected from the group consisting of E, H, S and Y;
—X(260)- is selected from the group consisting of T, D, E, H and Y;
—X(262)- is selected from the group consisting of V, A, E, F, I and T;
—X(263)- is selected from the group consisting of V, A, I, M and T;
—X(264)- is selected from the group consisting of V, A, D, E, F, G, H, I, K, L, M, N, P, Q, R, S, T, W and Y;
—X(265)- is selected from the group consisting of D, F, G, H, I, K, L, M, P, Q, R, S, T, V, W and Y;
—X(266)- is selected from the group consisting of V, A, I, M and T;
—X(267)- is selected from the group consisting of S, D, E, F, H, I, K, L, M, N, P, Q, R, V, W and Y;
—X(268)- is selected from the group consisting of H, D, E, F, G, I, K, L, M, P, R, T, V and W;
—X(269)- is selected from the group consisting of E, F, G, H, I, K, L, M, N, P, R, S, T, V, W and Y;
—X(270)- is selected from the group consisting of D, F, G, H, I, L, M, P, Q, R, S, T, W and Y;
—X(271)- is selected from the group consisting of P, A, D, E, F, G, H, I, K, L, M, N, Q, R, S, T, V, W and Y;
—X(272)- is selected from the group consisting of E, D, F, G, H, I, K, L, M, P, R, S, T, V, W and Y;
—X(273)- is selected from the group consisting of V and I;
—X(274)- is selected from the group consisting of Q, D, E, F, G, H, I, L, M, N, P, R, T, V, W and Y;
—X(275)- is selected from the group consisting of F, L and W;
—X(276)- is selected from the group consisting of K, D, E, F, G, H, I, L, M, P, R, S, T, V, W and Y;
—X(278)- is selected from the group consisting of Y, D, E, G, H, I, K, L, M, N, P, Q, R, S, T, V and W;
—X(280)- is selected from the group consisting of D, G, K, L, P and W;
—X(281)- is selected from the group consisting of G, D, E, K, N, P, Q and Y;
—X(282)- is selected from the group consisting of V, E, G, K, P and Y;
—X(283)- is selected from the group consisting of E, G, H, K, L, P, R and Y;
—X(284)- is selected from the group consisting of V, D, E, L, N, Q, T and Y;
—X(285)- is selected from the group consisting of H, D, E, K, Q, W and Y;
—X(286)- is selected from the group consisting of N, E, G, P and Y;
—X(288)- is selected from the group consisting of K, D, E and Y;
—X(290)- is selected from the group consisting of K, D, H, L, N and W;
—X(291)- is selected from the group consisting of P, D, E, G, H, I, Q and T;
—X(292)- is selected from the group consisting of R, D, E, T and Y;
—X(293)- is selected from the group consisting of E, F, G, H, I, L, M, N, P, R, S, T, V, W and Y;
—X(294)- is selected from the group consisting of E, F, G, H, I, K, L, M, P, R, S, T, V, W and Y;
—X(295)- is selected from the group consisting of Q, D, E, F, G, H, I, M, N, P, R, S, T, V, W and Y;
—X(296)- is selected from the group consisting of Y, A, D, E, G, I, K, L, M, N, Q, R, S, T and V;
—X(297)- is selected from the group consisting of N, D, E, F, G, H, I, K, L, M, P, Q, R, S, T, V, W and Y;
—X(298)- is selected from the group consisting of S, E, F, H, I, K, M, Q, R, W and Y;
—X(299)- is selected from the group consisting of T, A, D, E, F, G, H, I, K, L, M, N, P, Q, R, S, V, W and Y;
—X(300)- is selected from the group consisting of F, A, D, E, G, H, K, M, N, P, Q, R, S, T, V and W;
—X(301)- is selected from the group consisting of R, D, E, H and Y;
—X(302)- is selected from the group consisting of V and I;
—X(303)- is selected from the group consisting of V, D, E and Y;
—X(304)- is selected from the group consisting of S, D, H, L, N and T;
—X(305)- is selected from the group consisting of V, E, T and Y;
—X(313)- is selected from the group consisting of W and F;
—X(317)- is selected from the group consisting of K, E and Q;
—X(318)- is selected from the group consisting of E, H, L, Q, R and Y;
—X(320)- is selected from the group consisting of K, D, F, G, H, I, L, N, P, S, T, V, W and Y;
—X(322)- is selected from the group consisting of K, D, F, G, H, I, P, S, T, V, W and Y;
—X(323)- is selected from the group consisting of V and I;
—X(324)- is selected from the group consisting of S, D, F, G, H, I, L, M, P, R, T, V, W and Y;
—X(325)- is selected from the group consisting of N, A, D, E, F, G, H, I, K, L, M, P, Q, R, S, T, V, W and Y;
—X(326)- is selected from the group consisting of K, I, L, P and T;
—X(327)- is selected from the group consisting of A, D, E, F, H, I, K, L, M, N, P, R, T, V, W and Y;
—X(328)- is selected from the group consisting of L, A, D, E, F, G, H, I, K, M, N, P, Q, R, S, T, V, W and Y;
—X(329)- is selected from the group consisting of P, D, E, F, G, H, I, K, L, M, N, Q, R, S, T, V, W and Y;
—X(330)- is selected from the group consisting of A, E, F, G, H, I, L, M, N, P, R, T, V, W and Y;
—X(331)- is selected from the group consisting of P, D, F, H, I, L, M, Q, R, T, V, W and Y;
—X(332)- is selected from the group consisting of I, A, D, E, F, H, K, L, M, N, P, Q, R, S, T, V, W and Y;
—X(333)- is selected from the group consisting of E, F, H, I, L, M, P, T and Y;
—X(334)- is selected from the group consisting of K, F, I, P and T;
—X(335)- is selected from the group consisting of T, D, F, G, H, I, L, M, N, P, R, S, V, W and Y;
—X(336)- is selected from the group consisting of I, E, K and Y; and
—X(337)- is selected from the group consisting of S, E, H and N.

In certain variations, the variant differs from SEQ ID NO:12 by at least one amino acid.

In another aspect, the present application is directed to an IgG3 variant including an amino acid sequence having the formula:

(SED ID NO: 103)
ASTKGPSVFPLAPCSRSTSGGTAALGCLVKDYFPEPVTVSWNSGALTSGVHTFPAVL
QSSGLYSLSSVVTVPSSSLGTQTYTCNVNHKPSNTKVDKRVELKTP-X(221)-
GDTTHTC-X(227)-
RCPEPKSCDTPPPCPRCPEPKSCDTPPPCPRCPEPKSCDTPPPCPRCPAPE-X(234)-
X(235)-X(236)-X(237)-P-X(239)-X(240)-FLFPP-X(246)-PKDTLMIS-X(255)-TP-X(258)-
V-X(260)-CVV-X(264)-DV-X(267)-X(268)-ED-X(271)-X(272)-V-X(274)-FKW-X(278)-
VD-X(281)-V-X(283)-X(284)-HNAKT-X(290)-PR-X(293)-E-X(295)-YNSTFRVV-X(304)-
VLTVLHQDWLNGKEYKCKV-X(324)-N-X(326)-X(327)-X(328)-P-X(330)-P-X(332)-
X(333)-X(334)-
TISKTKGQPREPQVYTLPPSREEMTKNQVSLTCLVKGFYPSDIAVEWESSGQPENNYN
TTPPMLDSDGSFFLYSKLT VDKSRWQQGNIFSCSVMHEALHNRFTQKSLSLSPGK-;

wherein
—X(221)- is selected from the group consisting of L and K;
—X(227)- is selected from the group consisting of P and G;
—X(234)- is selected from the group consisting of L, Y and I;
—X(235)- is selected from the group consisting of L, Y, I and D;
—X(236)- is selected from the group consisting of G, S and A;
—X(237)- is selected from the group consisting of G and D;
—X(239)- is selected from the group consisting of S, D, E, N, Q and T;
—X(240)- is selected from the group consisting of V, I and M;
—X(246)- is selected from the group consisting of K, H and Y;
—X(255)- is selected from the group consisting of R and Y;
—X(258)- is selected from the group consisting of E, H and Y;
—X(260)- is selected from the group consisting of T and H;
—X(264)- is selected from the group consisting of V, I, T and Y;
—X(267)- is selected from the group consisting of S, D and E;
—X(268)- is selected from the group consisting of H, D and E;
—X(271)- is selected from the group consisting of P and G;
—X(272)- is selected from the group consisting of E, Y, H, R and I;
—X(274)- is selected from the group consisting of Q and E;
—X(278)- is selected from the group consisting of Y and T;
—X(281)- is selected from the group consisting of G, D and E;
—X(283)- is selected from the group consisting of E, L and H;
—X(284)- is selected from the group consisting of V, E and D;
—X(290)- is selected from the group consisting of K and N;
—X(293)- is selected from the group consisting of E and R;
—X(295)- is selected from the group consisting of Q and E;
—X(304)- is selected from the group consisting of S and T;
—X(324)- is selected from the group consisting of S, G and I;
—X(326)- is selected from the group consisting of K and T;
—X(327)- is selected from the group consisting of A and D;
—X(328)- is selected from the group consisting of L, A, F, I and T;
—X(330)- is selected from the group consisting of A, L, Y and I;
—X(332)- is selected from the group consisting of I, D, E, N, Q and T;
—X(333)- is selected from the group consisting of E and Y; and
—X(334)- is selected from the group consisting of K, F, I and T.

In certain variations, the variant differs from SEQ ID NO:12 by at least one amino acid. In additional variations, the formula has at least 1, 2, 3, 4, 5, 6, 7, 8, 9, 10 or more amino acid modifications as compared to an amino acid sequence including SEQ ID NO:12. In additional embodiments, at least 2, 3, or 4 of the modifications are in different domains.

In another aspect, the present application is directed to an IgG4 variant including two or more amino acid modifications as compared to SEQ ID NO:13. The modifications can be selected from among C131S, R133K, E137G, S138G, S192N, L193F, K196Q, T199I, D203N, R214K, R214T, S217P, S217R, S217L, Y219S, Y219C, Y219T, G220C, G220P, -221D, -221L, insertion of the sequence LGD at -221, -222K, -222V, -222T, -223T, P224H, P224E, P225T, P225-, S228P, S228R, substitution of the sequence RCPEPKSCDTPPPCPRCPEPKSCDTPPPCPRCPEPKSCDTPPPCPR at 228, E233P, F234L, F234V, L235A, G236-, Q268H, Q274K, N276K, F296Y, Y300F, L309V, G327A, S330A, S331P, A339T, Q355R, E356D, M358L, N384S, K392N, V397M, R409K, E419Q, V422I, H435R, Y436F, and L445P. In certain embodiments, at least two of the amino acid modifications are in different domains. In various embodiments, the formula has at least 1, 2, 3, 4, 5, 6, 7, 8, 9, 10 or more amino acid modifications as compared to an amino acid sequence including SEQ ID NO:14. In additional embodiments, at least 2, 3, or 4 of the modifications are in different domains.

In another aspect, the present application is directed to an IgG4 variant including an amino acid sequence having the formula:

(SED ID NO: 104)

ASTKGPSVFPLAP-X(131)-S-X(133)-STS-X(137)-X(138)-TA
ALGCLVKDYFPEPVTVSWNSGALTSGVHTFPAVLQSSGLYSLSSVVTV
PSSSLGT-X(196)-TY-X(199)-CNV-X(203)-HKPSNTKVDK-
X(214)-VE-X(217)-K-X(219)-X(220)-X(221)-X(222)-
X(223)-X(224)-X(225)-CP-X(228)-CPAPE-X(234)-LGGPS
VFLFPPKPKDTLMISRTPEVTCVVVDVS-X(268)-EDPEV-X(274)-
FNWYVDGVEVHNAKTKPREEQ-X(296)-NSTYRVVSVLTVLHQDWLNG
KEYKCKVSNK-X(327)-LP-X(330)-X(331)-IEKTISKAKGQPRE
PQVYTLPPS-X(355)-X(356)-E-X(358)-TKNQVSLTCLVKGFYP
SDIAVEWESNGQPENNYKTTPPVLDSDGSFFLYS-X(409)-LTVDKSR
WQ-X(419)-GNVFSCSVMHEALHNHYTQKSLSLS-X(445)-GK,

wherein
X(131) is selected from the group consisting of C and S;
X(133) is selected from the group consisting of R and K;
X(137) is selected from the group consisting of E and G;
X(138) is selected from the group consisting of S and G;
X(196) is selected from the group consisting of K and Q;
X(199) is selected from the group consisting of T and I;
X(203) is selected from the group consisting of D and N;
X(214) is selected from the group consisting of R and K;
X(217) is selected from the group consisting of S and P;
X(219) is selected from the group consisting of Y and S;
X(220) is selected from the group consisting of G and C;
X(221) is selected from the group consisting of no amino acid and D;
X(222) is selected from the group consisting of no amino acid and K;
X(223) is selected from the group consisting of no amino acid and T;
X(224) is selected from the group consisting of P and H;
X(225) is selected from the group consisting of P and T;
X(228) is selected from the group consisting of S and P;
X(234) is selected from the group consisting of F and L;
X(268) is selected from the group consisting of Q and H;
X(274) is selected from the group consisting of Q and K;
X(296) is selected from the group consisting of F and Y;
X(327) is selected from the group consisting of G and A;
X(330) is selected from the group consisting of S and A;
X(331) is selected from the group consisting of S and P;
X(355) is selected from the group consisting of Q and R;
X(356) is selected from the group consisting of E and D;
X(358) is selected from the group consisting of M and L;
X(409) is selected from the group consisting of R and K;
X(419) is selected from the group consisting of E and Q; and
X(445) is selected from the group consisting of L and P.

In certain embodiments, at least two of the amino acid modifications are in different domains. In various embodiments, the formula has at least 1, 2, 3, 4, 5, 6, 7, 8, 9, 10 or more amino acid modifications as compared to an amino acid sequence including SEQ ID NO:13. In additional embodiments, at least 2, 3, or 4 of the modifications are in different domains.

In another aspect, the present application is directed to an IgG4 variant including two or more amino acid modifications as compared to SEQ ID NO:14. In certain embodiments, the modifications selected from among C131S, R133K, E137G, S138G, K196Q, T199I, D203N, R214K, S217P, Y219S, G220C, 221D, -222K, -223T, P224H, P225T, S228P, F234L, Q268H, Q274K, F296Y, G327A, S330A, S331P, Q355R, E356D, M358L, R409K, E419Q, and L445P. In various embodiments, the formula has at least 1, 2, 3, 4, 5, 6, 7, 8, 9, 10 or more amino acid modifications as compared to an amino acid sequence including SEQ ID NO:13. In additional embodiments, at least 2, 3, or 4 of the modifications are in different domains.

In another aspect, the present application is directed to an IgG4 variant including an

(SED ID NO: 105)

-ASTKGPSVFPLAP-X(131)-S-X(133)-STS-X(137)-X(138)-T
AALGCLVKDYFPEPVTVSWNSGALTSGVHTFPAVLQSSGLYSLSSVVTVP
SS-X(192)-X(193)-GT-X(196)-TY-X(199)-CNV-X(203)-HK
PSNTKVDK-X(214)-VE-X(217)-K-X(219)-X(220)-X(221)-
X(222)-X(223)-X(224)-X(225)-C-X(227)-X(228)-C-
X(230)-X(231)-X(232)-X(233)-X(234)-X(235)-X(236)-
X(237)-X(238)-X(239)-X(240)-X(241)-L-X(243)-
X(244)-X(245)-X(246)-X(247)-K-X(249)-TLMIS-X(255)-
TP-X(258)-V-X(260)-C-X(262)-X(263)-X(264)-X(265)-
X(266)-X(267)-X(268)-X(269)-X(270)-X(271)-X(272)-
X(273)-X(274)-X(275)-X(276)-W-X(278)-V-X(280)-
X(281)-X(282)-X(283)-X(284)-X(285)-X(286)-A-
X(288)-T-X(290)-X(291)-X(292)-X(293)-X(294)-
X(295)-X(296)-X(297)-X(298)-X(299)-X(300)-X(301)-
X(302)-X(303)-X(304)-X(305)-LTV-X(309)-HQD-X(313)-
LNG-X(317)-X(318)-Y-X(320)-C-X(322)-X(323)-X(324)-
X(325)-X(326)-X(327)-X(328)-X(329)-X(330)-X(331)-
X(332)-X(333)-X(334)-X(335)-X(336)-X(337)-K-
X(339)-KGQPREPQVYTLPPS-X(355)-X(356)-E-X(358)-TKN
QVSLTCLVKGFYPSDIAVEWES-X(384)-GQPENNY-X(392)-TTPP-
X(397)-LDSDGSFFLYS-X(409)-LTVDKSRWQ-X(419)-GN-
X(422)-FSCSVMHEALHN-X(435)-X(436)-TQKSLSLS-X(445)-
GK,

amino acid sequence having the formula:
wherein
—X(131)- is selected from the group consisting of C and S;
—X(133)- is selected from the group consisting of R and K;
—X(137)- is selected from the group consisting of E and G;
—X(138)- is selected from the group consisting of S and G;
—X(192)- is selected from the group consisting of N and S;
—X(193)- is selected from the group consisting of F and L;
—X(196)- is selected from the group consisting of Q and K;
—X(199)- is selected from the group consisting of T and I;
—X(203)- is selected from the group consisting of D and N;
—X(214)- is selected from the group consisting of T, K and R;
—X(217)- is selected from the group consisting of R, P, L and S;
—X(219)- is selected from the group consisting of C, S, T and Y;
—X(220)- is selected from the group consisting of C, P and G;
—X(221)- is selected from the group consisting of no amino acid, D, K, Y, L, and the sequence LGD;
—X(222)- is selected from the group consisting of V, K, T, no amino acid, E and Y;
—X(223)- is selected from the group consisting of no amino acid, T, E and K;
—X(224)- is selected from the group consisting of E, H, P and Y;
—X(225)- is selected from the group consisting of no amino acid, T, P, E, K and W;
—X(227)- is selected from the group consisting of P, E, G, K and Y;
—X(228)- is selected from the group consisting of P, S, E, G, K, Y, R, and the sequence

(SEQ ID NO: 111)

RCPEPKSCDTPPPCPRCPEPKSCDTPPPCPRCPEPKSCDTPPPCPR;

—X(230)- is selected from the group consisting of P, A, E, G and Y;
—X(231)- is selected from the group consisting of A, E, G, K, P and Y;
—X(232)- is selected from the group consisting of P, E, G, K and Y;
—X(233)- is selected from the group consisting of P, E, A, D, F, G, H, I, K, L, M, N, Q, R, S, T, V, W and Y;
—X(234)- is selected from the group consisting of V, L, F, D, E, F, G, H, I, K, M, N, P, Q, R, S, T, W and Y;
—X(235)- is selected from the group consisting of A, L, D, F, G, H, I, K, M, N, P, Q, R, S, T, V, W, and Y;
—X(236)- is selected from the group consisting of no amino acid, G, A, D, E, F, H, I, K, L, M, N, P, Q, R, S, T, V, W and Y;
—X(237)- is selected from the group consisting of G, D, E, F, H, I, K, L, M, N, P, Q, R, S, T, V, W and Y;
—X(238)- is selected from the group consisting of P, D, E, F, G, H, I, K, L, M, N, Q, R, S, T, V, W and Y;
—X(239)- is selected from the group consisting of S, D, E, F, G, H, I, K, L, M, N, P, Q, R, T, V, W and Y;
—X(240)- is selected from the group consisting of V, A, I, M and T;
—X(241)- is selected from the group consisting of F, D, E, L, R, S, W and Y;
—X(243)- is selected from the group consisting of F, E, H, L, Q, R, W and Y;
—X(244)- is selected from the group consisting of P and H;
—X(245)- is selected from the group consisting of P and A;
—X(246)- is selected from the group consisting of, K, D, E, H and Y;
—X(247)- is selected from the group consisting of P, G and V;
—X(249)- is selected from the group consisting of D, H, Q and Y;
—X(255)- is selected from the group consisting of R, E and Y;
—X(258)- is selected from the group consisting of E, H, S and Y;
—X(260)- is selected from the group consisting of T, D, E, H and Y;
—X(262)- is selected from the group consisting of V, A, E, F, I and T;
—X(263)- is selected from the group consisting of V, A, I, M and T;
—X(264)- is selected from the group consisting of V, A, D, E, F, G, H, I, K, L, M, N, P, Q, R, S, T, W, and Y;
—X(265)- is selected from the group consisting of D, F, G, H, I, K, L, M, P, Q, R, S, T, V, W and Y;
—X(266)- is selected from the group consisting of V, A, I, M and T;
—X(267)- is selected from the group consisting of S, D, E, F, H, I, K, L, M, N, P, Q, R, V, W and Y;
—X(268)- is selected from the group consisting of H, Q, D, E, F, G, I, K, L, M, P, R, T, V and W;
—X(269)- is selected from the group consisting of E, F, G, H, I, K, L, M, N, P, R, S, T, V, W and Y;
—X(270)- is selected from the group consisting of D, F, G, H, I, L, M, P, Q, R, S, T, W and Y;
—X(271)- is selected from the group consisting of P, A, D, E, F, G, H, I, K, L, M, N, Q, R, S, T, V, W and Y;
—X(272)- is selected from the group consisting of E, D, F, G, H, I, K, L, M, P, R, S, T, V, W and Y;
—X(273)- is selected from the group consisting of V and I;
—X(274)- is selected from the group consisting of Q, K, D, E, F, G, H, I, L, M, N, P, R, T, V, W and Y;
—X(275)- is selected from the group consisting of F, L and W;
—X(276)- is selected from the group consisting of N, K, D, E, F, G, H, I, L, M, P, R, S, T, V, W and Y;
—X(278)- is selected from the group consisting of Y, D, E, G, H, I, K, L, M, N, P, Q, R, S, T, V and W;
—X(280)- is selected from the group consisting of D, G, K, L, P and W;
—X(281)- is selected from the group consisting of G, D, E, K, N, P, Q and Y;
—X(282)- is selected from the group consisting of V, E, G, K, P and Y;
—X(283)- is selected from the group consisting of E, G, H, K, L, P, R and Y;
—X(284)- is selected from the group consisting of V, D, E, L, N, Q, T and Y;
—X(285)- is selected from the group consisting of H, D, E, K, Q, W and Y;
—X(286)- is selected from the group consisting of N, E, G, P and Y;
—X(288)- is selected from the group consisting of K, D, E and Y;
—X(290)- is selected from the group consisting of K, D, H, L, N and W;
—X(291)- is selected from the group consisting of P, D, E, G, H, I, Q and T;
—X(292)- is selected from the group consisting of R, D, E, T and Y;
—X(293)- is selected from the group consisting of E, F, G, H, I, L, M, N, P, R, S, T, V, W and Y;
—X(294)- is selected from the group consisting of E, F, G, H, I, K, L, M, P, R, S, T, V, W and Y;
—X(295)- is selected from the group consisting of Q, D, E, F, G, H, I, M, N, P, R, S, T, V, W and Y;
—X(296)- is selected from the group consisting of F, Y, A, D, E, G, I, K, L, M, N, Q, R, S, T and V;
—X(297)- is selected from the group consisting of N, D, E, F, G, H, I, K, L, M, P, Q, R, S, T, V, W and Y;
—X(298)- is selected from the group consisting of S, E, F, H, I, K, M, Q, R, W and Y;
—X(299)- is selected from the group consisting of T, A, D, E, F, G, H, I, K, L, M, N, P, Q, R, S, V, W and Y;
—X(300)- is selected from the group consisting of F, Y, A, D, E, G, H, K, M, N, P, Q, R, S, T, V and W;
—X(301)- is selected from the group consisting of R, D, E, H and Y;
—X(302)- is selected from the group consisting of V and I;
—X(303)- is selected from the group consisting of V, D, E and Y;
—X(304)- is selected from the group consisting of S, D, H, L, N and T;
—X(305)- is selected from the group consisting of V, E, T and Y;
—X(309)- is selected from the group consisting of V and L;
—X(313)- is selected from the group consisting of W and F;
—X(317)- is selected from the group consisting of K, E and Q;
—X(318)- is selected from the group consisting of E, H, L, Q, R and Y;
—X(320)- is selected from the group consisting of K, D, F, G, H, I, L, N, P, S, T, V, W and Y;
—X(322)- is selected from the group consisting of K, D, F, G, H, I, P, S, T, V, W and Y;
—X(323)- is selected from the group consisting of V and I;
—X(324)- is selected from the group consisting of S, D, F, G, H, I, L, M, P, R, T, V, W and Y;
—X(325)- is selected from the group consisting of N, A, D, E, F, G, H, I, K, L, M, P, Q, R, S, T, V, W and Y;
—X(326)- is selected from the group consisting of K, I, L, P and T;
—X(327)- is selected from the group consisting of G, A, D, E, F, H, I, K, L, M, N, P, R, T, V, W and Y;
—X(328)- is selected from the group consisting of L, A, D, E, F, G, H, I, K, M, N, P, Q, R, S, T, V, W and Y;
—X(329)- is selected from the group consisting of P, D, E, F, G, H, I, K, L, M, N, Q, R, S, T, V, W and Y;
—X(330)- is selected from the group consisting of A, S, E, F, G, H, I, L, M, N, P, R, T, V, W and Y;
—X(331)- is selected from the group consisting of P, S, D, F, H, I, L, M, Q, R, T, V, W and Y;
—X(332)- is selected from the group consisting of I, A, D, E, F, H, K, L, M, N, P, Q, R, S, T, V, W and Y;
—X(333)- is selected from the group consisting of E, F, H, I, L, M, P, T and Y;
—X(334)- is selected from the group consisting of K, F, I, P and T;
—X(335)- is selected from the group consisting of T, D, F, G, H, I, L, M, N, P, R, S, V, W and Y;
—X(336)- is selected from the group consisting of I, E, K and Y;
—X(337)- is selected from the group consisting of S, E, H and N;
—X(339)- is selected from the group consisting of T and A;
—X(355)- is selected from the group consisting of R and Q;
—X(356)- is selected from the group consisting of E and D;
—X(358)- is selected from the group consisting of M and L;
—X(384)- is selected from the group consisting of N and S;
—X(392)- is selected from the group consisting of K and N;
—X(397)- is selected from the group consisting of M and V;
—X(409)- is selected from the group consisting of K and R;
—X(419)- is selected from the group consisting of Q and E;
—X(422)- is selected from the group consisting of V and I;
—X(435)- is selected from the group consisting of H and R;
—X(436)- is selected from the group consisting of Y and F; and
—X(445)- is selected from the group consisting of P and L.

In one variation, a first modification is selected from among C131S, R133K, E137G, S138G, S192N, L193F, K196Q, T199I, D203N, R214K, R214T, S217P, S217R, S217L, Y219S, Y219C, Y219T, G220C, G220P, -221D, -221L, insertion of the sequence LGD at -221, -222K, -222V, -222T, -223T, P224H, P224E, P225T, P225-, S228P, S228R, substitution of the sequence RCPEPKSCDTPPPCPRCPEPKSCDTPPPCPRCPEPKSCDTPPPCPR (SEQ ID NO: 111) at 228, E233P, F234L, F234V, L235A, G236-, Q268H, Q274K, N276K, F296Y, Y300F, L309V, G327A, S330A, S331P, A339T, Q355R, E356D, M358L, N384S, K392N, V397M, R409K, E419Q, V422I, H435R, Y436F, and L445P. In a further variation, a second modification is selected from among 221K, 221Y, 222E, 222Y, 223E, 223K, 224Y, 225E, 225K, 225W, 227E, 227G, 227K, 227Y, 228E, 228G, 228K, 228Y, 230A, 230E, 230G, 230Y, 231E, 231G, 231K, 231P, 231Y, 232E, 232G, 232K, 232Y, 233A, 233D, 233F, 233G, 233H, 233I, 233K, 233L, 233M, 233N, 233Q, 233R, 233S, 233T, 233V, 233W, 233Y, 234D, 234E, 234F, 234G, 234H, 234I, 234K, 234M, 234N, 234P, 234Q, 234R, 234S, 234T, 234W, 234Y, 235D, 235F, 235G, 235H, 235I, 235K, 235M, 235N, 235P, 235Q, 235R, 235S, 235T, 235V, 235W, 235Y, 236A, 236D, 236E, 236F, 236H, 236I, 236K, 236L, 236M, 236N, 236P, 236Q, 236R, 236S, 236T, 236V, 236W, 236Y, 237D, 237E, 237F, 237H, 237I, 237K, 237L, 237M, 237N, 237P, 237Q, 237R, 237S, 237T, 237V, 237W, 237Y, 238D, 238E, 238F, 238G, 238H, 238I, 238K, 238L, 238M, 238N, 238Q, 238R, 238S, 238T, 238V, 238W, 238Y, 239D, 239E, 239F, 239G, 239H, 239I, 239K, 239L, 239M, 239N, 239P, 239Q, 239R, 239T, 239V, 239W, 239Y, 240A, 240I, 240M, 240T, 241D, 241E, 241L, 241R, 241S, 241W, 241Y, 243E, 243H, 243L, 243Q, 243R, 243W, 243Y, 244H, 245A, 246D, 246E, 246H, 246Y, 247G, 247V, 249H, 249Q, 249Y, 255E, 255Y, 258H, 258S, 258Y, 260D, 260E, 260H, 260Y, 262A, 262E, 262F, 262I, 262T, 263A, 263I, 263M, 263T, 264A, 264D, 264E, 264F, 264G, 264H, 264I, 264K, 264L, 264M, 264N, 264P, 264Q, 264R, 264S, 264T, 264W, 264Y, 265F, 265G, 265H, 265I, 265K, 265L, 265M, 265P, 265Q, 265R, 265S, 265T, 265V, 265W, 265Y, 266A, 266I, 266M, 266T, 267D, 267E, 267F, 267H, 267I, 267K, 267L, 267M, 267N, 267P, 267Q, 267R, 267V, 267W, 267Y, 268D, 268E, 268F, 268G, 268I, 268K, 268L, 268M, 268P, 268R, 268T, 268V, 268W, 269F, 269G, 269H, 269I, 269K, 269L, 269M, 269N, 269P, 269R, 269S, 269T, 269V, 269W, 269Y, 270F, 270G, 270H, 270I, 270L, 270M, 270P, 270Q, 270R, 270S, 270T, 270W, 270Y, 271A, 271D, 271E, 271F, 271G, 271H, 271I, 271K, 271L, 271M, 271N, 271Q, 271R, 271S, 271T, 271V, 271W, 271Y, 272D, 272F, 272G, 272H, 272I, 272K, 272L, 272M, 272P, 272R, 272S, 272T, 272V, 272W, 272Y, 273I, 274D, 274E, 274F, 274G, 274H, 274I, 274L, 274M, 274N, 274P, 274R, 274T, 274V, 274W, 274Y, 275L, 275W, 276D, 276E, 276F, 276G, 276H, 276I, 276L, 276M, 276P, 276R, 276S, 276T, 276V, 276W, 276Y, 278D, 278E, 278G, 278H, 278I, 278K, 278L, 278M, 278N, 278P, 278Q, 278R, 278S, 278T, 278V, 278W, 280G, 280K, 280L, 280P, 280W, 281D, 281E, 281K, 281N, 281P, 281Q, 281Y, 282E, 282G, 282K, 282P, 282Y, 283G, 283H, 283K, 283L, 283P, 283R, 283Y, 284D, 284E, 284L, 284N, 284Q, 284T, 284Y, 285D, 285E, 285K, 285Q, 285W, 285Y, 286E, 286G, 286P, 286Y, 288D, 288E, 288Y, 290D, 290H, 290L, 290N, 290W, 291D, 291E, 291G, 291H, 291I, 291Q, 291T, 292D, 292E, 292T, 292Y, 293F, 293G, 293H, 293I, 293L, 293M, 293N, 293P, 293R, 293S, 293T, 293V, 293W, 293Y, 294F, 294G, 294H, 294I, 294K, 294L, 294M, 294P, 294R, 294S, 294T, 294V, 294W, 294Y, 295D, 295E, 295F, 295G, 295H, 295I, 295M, 295N, 295P, 295R, 295S, 295T, 295V, 295W, 295Y, 296A, 296D, 296E, 296G, 296I, 296K, 296L, 296M, 296N, 296Q, 296R, 296S, 296T, 296V, 297D, 297E, 297F, 297G, 297H, 297I, 297K, 297L, 297M, 297P, 297Q, 297R, 297S, 297T, 297V, 297W, 297Y, 298E, 298F, 298H, 298I, 298K, 298M, 298Q, 298R, 298W, 298Y, 299A, 299D, 299E, 299F, 299G, 299H, 299I, 299K, 299L, 299M, 299N, 299P, 299Q, 299R, 299S, 299V, 299W, 299Y, 300A, 300D, 300E, 300G, 300H, 300K, 300M, 300N, 300P, 300Q, 300R, 300S, 300T, 300V, 300W, 301D, 301E, 301H, 301Y, 302I, 303D, 303E, 303Y, 304D, 304H, 304L, 304N, 304T, 305E, 305T, 305Y, 313F, 317E, 317Q, 318H, 318L, 318Q, 318R, 318Y, 320D, 320F, 320G, 320H, 320I, 320L, 320N, 320P, 320S, 320T, 320V, 320W, 320Y, 322D, 322F, 322G, 322H, 322I, 322P, 322S, 322T, 322V, 322W, 322Y, 323I, 324D, 324F, 324G, 324H, 324I, 324L, 324M, 324P, 324R, 324T, 324V, 324W, 324Y, 325A, 325D, 325E, 325F, 325G, 325H, 325I, 325K, 325L, 325M, 325P, 325Q, 325R, 325S, 325T, 325V, 325W, 325Y, 326I, 326L, 326P, 326T, 327D, 327E, 327F, 327H, 327I, 327K, 327L, 327M, 327N, 327P, 327R, 327T, 327V, 327W, 327Y, 328A, 328D, 328E, 328F, 328G, 328H, 328I, 328K, 328M, 328N, 328P, 328Q, 328R, 328S, 328T, 328V, 328W, 328Y, 329D, 329E, 329F, 329G, 329H, 329I, 329K, 329L, 329M, 329N, 329Q, 329R, 329S, 329T, 329V, 329W, 329Y, 330E, 330F, 330G, 330H, 330I, 330L, 330M, 330N, 330P, 330R, 330T, 330V, 330W, 330Y, 331D, 331F, 331H, 331I, 331L, 331M, 331Q, 331R, 331T, 331V, 331W, 331Y, 332A, 332D, 332E, 332F, 332H, 332K, 332L, 332M, 332N, 332P, 332Q, 332R, 332S, 332T, 332V, 332W, 332Y, 333F, 333H, 333I, 333L, 333M, 333P, 333T, 333Y, 334F, 334I, 334P, 334T, 335D, 335F, 335G, 335H, 335I, 335L, 335M, 335N, 335P, 335R, 335S, 335V, 335W, 335Y, 336E, 336K, 336Y, 337E, 337H, and 337N.

In a further aspect, the present application is directed to an IgG4 variant amino acid sequence having at least two amino acid modifications as compared to SEQ ID NO:13. The IgG4 variant includes a first modification selected from among C131S, R133K, E137G, S138G, S192N, L193F, K196Q, T199I, D203N, R214K, R214T, S217P, S217R, S217L, Y219S, Y219C, Y219T, G220C, G220P, -221D, -221L, insertion of the sequence LGD at -221, -222K, -222V, -222T, -223T, P224H, P224E, P225T, P225-, S228P, S228R, substitution of the sequence RCPEPKSCDTPPPCPRCPEPKSCDTPPPCPRCPEPKSCDTPPPCPR (SEQ ID NO: 111) at 228, E233P, F234L, F234V, L235A, G236-, Q268H, Q274K, N276K, F296Y, Y300F, L309V, G327A, S330A, S331P, A339T, Q355R, E356D, M358L, N384S, K392N, V397M, R409K, E419Q, V422I, H435R, Y436F, and L445P. In a further variation, a second modification is selected from among 221K, 221Y, 222E, 222Y, 223E, 223K, 224Y, 225E, 225K, 225W, 227E, 227G, 227K, 227Y, 228E, 228G, 228K, 228Y, 230A, 230E, 230G, 230Y, 231E, 231G, 231K, 231P, 231Y, 232E, 232G, 232K, 232Y, 233A, 233D, 233F, 233G, 233H, 233I, 233K, 233L, 233M, 233N, 233Q, 233R, 233S, 233T, 233V, 233W, 233Y, 234D, 234E, 234F, 234G, 234H, 234I, 234K, 234M, 234N, 234P, 234Q, 234R, 234S, 234T, 234W, 234Y, 235D, 235F, 235G, 235H, 235I, 235K, 235M, 235N, 235P, 235Q, 235R, 235S, 235T, 235V, 235W, 235Y, 236A, 236D, 236E, 236F, 236H, 236I, 236K, 236L, 236M, 236N, 236P, 236Q, 236R, 236S, 236T, 236V, 236W, 236Y, 237D, 237E, 237F, 237H, 237I, 237K, 237L, 237M, 237N, 237P, 237Q, 237R, 237S, 237T, 237V, 237W, 237Y, 238D, 238E, 238F, 238G, 238H, 238I, 238K, 238L, 238M, 238N, 238Q, 238R, 238S, 238T, 238V, 238W, 238Y, 239D, 239E, 239F, 239G, 239H, 239I, 239K, 239L, 239M, 239N, 239P, 239Q, 239R, 239T, 239V, 239W, 239Y, 240A, 240I, 240M, 240T, 241D, 241E, 241L, 241R, 241S, 241W, 241Y, 243E, 243H, 243L, 243Q, 243R, 243W, 243Y, 244H, 245A, 246D, 246E, 246H, 246Y, 247G, 247V, 249H, 249Q, 249Y, 255E, 255Y, 258H, 258S, 258Y, 260D, 260E, 260H, 260Y, 262A, 262E, 262F, 262I, 262T, 263A, 263I, 263M, 263T, 264A, 264D, 264E, 264F, 264G, 264H, 264I, 264K, 264L, 264M, 264N, 264P, 264Q, 264R, 264S, 264T, 264W, 264Y, 265F, 265G, 265H, 265I, 265K, 265L, 265M, 265P, 265Q, 265R, 265S, 265T, 265V, 265W, 265Y, 266A, 266I, 266M, 266T, 267D, 267E, 267F, 267H, 267I, 267K, 267L, 267M, 267N, 267P, 267Q, 267R, 267V, 267W, 267Y, 268D, 268E, 268F, 268G, 268I, 268K, 268L, 268M, 268P, 268R, 268T, 268V, 268W, 269F, 269G, 269H, 269I, 269K, 269L, 269M, 269N, 269P, 269R, 269S, 269T, 269V, 269W, 269Y, 270F, 270G, 270H, 270I, 270L, 270M, 270P, 270Q, 270R, 270S, 270T, 270W, 270Y, 271A, 271D, 271E, 271F, 271G, 271H, 271I, 271K, 271L, 271M, 271N, 271Q, 271R, 271S, 271T, 271V, 271W, 271Y, 272D, 272F, 272G, 272H, 272I, 272K, 272L, 272M, 272P, 272R, 272S, 272T, 272V, 272W, 272Y, 273I, 274D, 274E, 274F, 274G, 274H, 274I, 274L, 274M, 274N, 274P, 274R, 274T, 274V, 274W, 274Y, 275L, 275W, 276D, 276E, 276F, 276G, 276H, 276I, 276L, 276M, 276P, 276R, 276S, 276T, 276V, 276W, 276Y, 278D, 278E, 278G, 278H, 278I, 278K, 278L, 278M, 278N, 278P, 278Q, 278R, 278S, 278T, 278V, 278W, 280G, 280K, 280L, 280P, 280W, 281D, 281E, 281K, 281N, 281P, 281Q, 281Y, 282E, 282G, 282K, 282P, 282Y, 283G, 283H, 283K, 283L, 283P, 283R, 283Y, 284D, 284E, 284L, 284N, 284Q, 284T, 284Y, 285D, 285E, 285K, 285Q, 285W, 285Y, 286E, 286G, 286P, 286Y, 288D, 288E, 288Y, 290D, 290H, 290L, 290N, 290W, 291D, 291E, 291G, 291H, 291I, 291Q, 291T, 292D, 292E, 292T, 292Y, 293F, 293G, 293H, 293I, 293L, 293M, 293N, 293P, 293R, 293S, 293T, 293V, 293W, 293Y, 294F, 294G, 294H, 294I, 294K, 294L, 294M, 294P, 294R, 294S, 294T, 294V, 294W, 294Y, 295D, 295E, 295F, 295G, 295H, 295I, 295M, 295N, 295P, 295R, 295S, 295T, 295V, 295W, 295Y, 296A, 296D, 296E, 296G, 296I, 296K, 296L, 296M, 296N, 296Q, 296R, 296S, 296T, 296V, 297D, 297E, 297F, 297G, 297H, 297I, 297K, 297L, 297M, 297P, 297Q, 297R, 297S, 297T, 297V, 297W, 297Y, 298E, 298F, 298H, 298I, 298K, 298M, 298Q, 298R, 298W, 298Y, 299A, 299D, 299E, 299F, 299G, 299H, 299I, 299K, 299L, 299M, 299N, 299P, 299Q, 299R, 299S, 299V, 299W, 299Y, 300A, 300D, 300E, 300G, 300H, 300K, 300M, 300N, 300P, 300Q, 300R, 300S, 300T, 300V, 300W, 301D, 301E, 301H, 301Y, 302I, 303D, 303E, 303Y, 304D, 304H, 304L, 304N, 304T, 305E, 305T, 305Y, 313F, 317E, 317Q, 318H, 318L, 318Q, 318R, 318Y, 320D, 320F, 320G, 320H, 320I, 320L, 320N, 320P, 320S, 320T, 320V, 320W, 320Y, 322D, 322F, 322G, 322H, 322I, 322P, 322S, 322T, 322V, 322W, 322Y, 323I, 324D, 324F, 324G, 324H, 324I, 324L, 324M, 324P, 324R, 324T, 324V, 324W, 324Y, 325A, 325D, 325E, 325F, 325G, 325H, 325I, 325K, 325L, 325M, 325P, 325Q, 325R, 325S, 325T, 325V, 325W, 325Y, 326I, 326L, 326P, 326T, 327D, 327E, 327F, 327H, 327I, 327K, 327L, 327M, 327N, 327P, 327R, 327T, 327V, 327W, 327Y, 328A, 328D, 328E, 328F, 328G, 328H, 328I, 328K, 328M, 328N, 328P, 328Q, 328R, 328S, 328T, 328V, 328W, 328Y, 329D, 329E, 329F, 329G, 329H, 329I, 329K, 329L, 329M, 329N, 329Q, 329R, 329S, 329T, 329V, 329W, 329Y, 330E, 330F, 330G, 330H, 330I, 330L, 330M, 330N, 330P, 330R, 330T, 330V, 330W, 330Y, 331D, 331F, 331H, 331I, 331L, 331M, 331Q, 331R, 331T, 331V, 331W, 331Y, 332A, 332D, 332E, 332F, 332H, 332K, 332L, 332M, 332N, 332P, 332Q, 332R, 332S, 332T, 332V, 332W, 332Y, 333F, 333H, 333I, 333L, 333M, 333P, 333T, 333Y, 334F, 334I, 334P, 334T, 335D, 335F, 335G, 335H, 335I, 335L, 335M, 335N, 335P, 335R, 335S, 335V, 335W, 335Y, 336E, 336K, 336Y, 337E, 337H, and 337N.

In another aspect, the present application is directed to an IgG4 variant including an amino acid sequence having the formula:

(SED ID NO: 106)

ASTKGPSVFPLAP-X(131)-S-X(133)-STS-X(137)-X(138)-TA
ALGCLVKDYFPEPVTVSWNSGALTSGVHTFPAVLQSSGLYSLSSVVTVPS
S-X(192)-X(193)-GT-X(196)-TY-X(199)-CNV-X(203)-HKP
SNTKVDK-X(214)-VE-X(217)-K-X(219)-X(220)-X(221)-
X(222)-X(223)-X(224)-X(225)-C-X(227)-X(228)-CPAP-
X(233)-X(234)-X(235)-X(236)-X(237)-P-X(239)-
X(240)-FLFPP-X(246)-PKDTLMIS-X(255)-TP-X(258)-V-
X(260)-CVV-X(264)-DV-X(267)-X(268)-ED-X(271)-
X(272)-V-X(274)-F-X(276)-W-X(278)-VD-X(281)-V-
X(283)-X(284)-HNAKT-X(290)-PR-X(293)-E-X(295)-
X(296)-NST-X(300)-RVV-X(304)-VLTV-X(309)-HQDWLNGK
EYKCKV-X(324)-N-X(326)-X(327)-X(328)-P-X(330)-
X(331)-X(332)-X(333)-X(334)-TISK-X(339)-KGQPREPQV
YTLPPS-X(355)-X(356)-E-X(358)-TKNQVSLTCLVKGFYPSDIA
VEWES-X(384)-GQPENNY-X(392)-TTPP-X(397)-LDSDGSFFLY
S-X(409)-LTVDKSRWQ-X(419)-GN-X(422)-FSCSVMHEALHN-
X(435)-X(436)-TQKSLSLS-X(445)-GK,

wherein
—X(131)- is selected from the group consisting of C and S;
—X(133)- is selected from the group consisting of R and K;
—X(137)- is selected from the group consisting of E and G;
—X(138)- is selected from the group consisting of S and G;
—X(192)- is selected from the group consisting of N and S;
—X(193)- is selected from the group consisting of F and L;
—X(196)- is selected from the group consisting of Q and K;
—X(199)- is selected from the group consisting of T and I;
—X(203)- is selected from the group consisting of D and N;
—X(214)- is selected from the group consisting of T, K and R;
—X(217)- is selected from the group consisting of R, P, L and S;
—X(219)- is selected from the group consisting of C, S, T and Y;
—X(220)- is selected from the group consisting of C, P and G;
—X(221)- is selected from the group consisting of no amino acid, D, L, K, and the sequence LGD;
—X(222)- is selected from the group consisting of V, K, T, and no amino acid;
—X(223)- is selected from the group consisting of no amino acid and T;
—X(224)- is selected from the group consisting of E, H and P;
—X(225)- is selected from the group consisting of no amino acid, T and P;
—X(227)- is selected from the group consisting of P and G;
—X(228)- is selected from the group consisting of P, R, S, and the sequence

(SEQ ID NO: 111)

RCPEPKSCDTPPPCPRCPEPKSCDTPPPCPRCPEPKSCDTPPPCPR;

—X(233)- is selected from the group consisting of P and E;
—X(234)- is selected from the group consisting of V, L, F, Y and I;
—X(235)- is selected from the group consisting of A, L, Y, I and D;
—X(236)- is selected from the group consisting of no amino acid, G, S and A;
—X(237)- is selected from the group consisting of G and D;
—X(239)- is selected from the group consisting of S, D, E, N, Q and T;
—X(240)- is selected from the group consisting of V, I and M;
—X(246)- is selected from the group consisting of K, H and Y;
—X(255)- is selected from the group consisting of R and Y;
—X(258)- is selected from the group consisting of E, H and Y;
—X(260)- is selected from the group consisting of T and H;
—X(264)- is selected from the group consisting of V, I, T and Y;
—X(267)- is selected from the group consisting of S, D and E;
—X(268)- is selected from the group consisting of H, Q, D and E;
—X(271)- is selected from the group consisting of P and G;
—X(272)- is selected from the group consisting of E, Y, H, R and I;
—X(274)- is selected from the group consisting of Q, K and E;
—X(276)- is selected from the group consisting of N and K;
—X(278)- is selected from the group consisting of Y and T;
—X(281)- is selected from the group consisting of G, D and E;
—X(283)- is selected from the group consisting of E, L and H;
—X(284)- is selected from the group consisting of V, E and D;
—X(290)- is selected from the group consisting of K and N;
—X(293)- is selected from the group consisting of E and R;
—X(295)- is selected from the group consisting of Q and E;
—X(296)- is selected from the group consisting of F and Y;
—X(300)- is selected from the group consisting of F and Y;
—X(304)- is selected from the group consisting of S and T;
—X(309)- is selected from the group consisting of V and L;
—X(324)- is selected from the group consisting of S, G and I;
—X(326)- is selected from the group consisting of K and T;
—X(327)- is selected from the group consisting of G, A and D;
—X(328)- is selected from the group consisting of L, A, F, I and T;
—X(330)- is selected from the group consisting of A, S, L, Y and I;
—X(331)- is selected from the group consisting of P and S;
—X(332)- is selected from the group consisting of I, D, E, N, Q and T;
—X(333)- is selected from the group consisting of E and Y;
—X(334)- is selected from the group consisting of K, F, I and T;
—X(339)- is selected from the group consisting of T and A;
—X(355)- is selected from the group consisting of R and Q;
—X(356)- is selected from the group consisting of E and D;
—X(358)- is selected from the group consisting of M and L;
—X(384)- is selected from the group consisting of N and S;
—X(392)- is selected from the group consisting of K and N;
—X(397)- is selected from the group consisting of M and V;
—X(409)- is selected from the group consisting of K and R;
—X(419)- is selected from the group consisting of Q and E;
—X(422)- is selected from the group consisting of V and I;
—X(435)- is selected from the group consisting of H and R;
—X(436)- is selected from the group consisting of Y and F; and
—X(445)- is selected from the group consisting of P and L.

In one variation, a first modification is selected from among C131S, R133K, E137G, S138G, S192N, L193F, K196Q, T199I, D203N, R214K, R214T, S217P, S217R, S217L, Y219S, Y219C, Y219T, G220C, G220P, -221D, -221L, insertion of the sequence LGD at -221, -222K, -222V, -222T, -223T, P224H, P224E, P225T, P225-, S228P, S228R, substitution of the sequence RCPEPKSCDTPPPCPRCPEPKSCDTPPPCPRCPEPKSCDTPPPCPR (SEQ ID NO: 111) at 228, E233P, F234L, F234V, L235A, G236-, Q268H, Q274K, N276K, F296Y, Y300F, L309V, G327A, S330A, S331P, A339T, Q355R, E356D, M358L, N384S, K392N, V397M, R409K, E419Q, V422I, H435R, Y436F, and L445P. In a further variation, a second modification is selected from among 227G, 234Y, 234I, 235Y, 235I, 235D, 236S, 236A, 237D, 239D, 239E, 239N, 239Q, 239T, 240I, 240M, 246H, 246Y, 255Y, 258H, 258Y, 260H, 264I, 264T, 264Y, 267D, 267E, 268D, 268E, 271G, 272Y, 272H, 272R, 272I, 274E, 278T, 281D, 281E, 283L, 283H, 284E, 284D, 290N, 293R, 295E, 304T, 324G, 324I, 326T, 327D, 328A, 328F, 328I, 328T, 330L, 330Y, 330I, 332D, 332E, 332N, 332Q, 332T, 333Y, 334F, 334I, and 334T.

In another aspect, the present application is directed to an IgG4 variant including an amino acid sequence having the formula:

(SED ID NO: 107)

-C-X(227)-X(228)-C-X(230)-X(231)-X(232)-X(233)-
X(234)-X(235)-X(236)--X(237)-X(238)-X(239)-
X(240)-X(241)-L-X(243)-X(244)-X(245)-X(246)-
X(247)-K-X(249)-TLMIS-X(255)-TP-X(258)-V-X(260)-
C-X(262)-X(263)-X(264)-X(265)-X(266)-X(267)-
X(268)-X(269)-X(270)-X(271)-X(272)-X(273)-X(274)-
X(275)-X(276)-W-X(278)-V-X(280)-X(281)-X(282)-
X(283)-X(284)-X(285)-X(286)-A-X(288)-T-X(290)-
X(291)-X(292)-X(293)-X(294)-X(295)-X(296)-X(297)-
X(298)-X(299)-X(300)-X(301)-X(302)-X(303)-X(304)-
X(305)-LTV-X(309)-HQD-X(313)-LNG-X(317)-X(318)-Y-
X(320)-C-X(322)-X(323)-X(324)-X(325)-X(326)-
X(327)-X(328)-X(329)-X(330)-X(331)-X(332)-X(333)-
X(334)-X(335)-X(336)-X(337)-K-X(339)-KGQPREPQVYTL
PPS-X(355)-X(356)-E-X(358)-TKNQVSLTCLVKGFYPSDIAVE
WES-X(384)-GQPENNY-X(392)-TTPP-X(397)-LDSDGSFFLYS-
X(409)-LTVDKSRWQ-X(419)-GN-X(422)-FSCSVMHEALHN-
X(435)-X(436)-TQKSLSLS-X(445)-GK,

wherein
—X(227)- is selected from the group consisting of P, E, G, K and Y;
—X(228)- is selected from the group consisting of P, S, E, G, K, Y, R, and the sequence

(SEQ ID NO: 111)

RCPEPKSCDTPPPCPRCPEPKSCDTPPPCPRCPEPKSCDTPPPCPR;

—X(230)- is selected from the group consisting of P, A, E, G and Y;
—X(231)- is selected from the group consisting of A, E, G, K, P and Y;
—X(232)- is selected from the group consisting of P, E, G, K and Y;
—X(233)- is selected from the group consisting of P, E, A, D, F, G, H, I, K, L, M, N, Q, R, S, T, V, W and Y;
—X(234)- is selected from the group consisting of V, L, F, D, E, F, G, H, I, K, M, N, P, Q, R, S, T, W and Y;
—X(235)- is selected from the group consisting of A, L, D, F, G, H, I, K, M, N, P, Q, R, S, T, V, W, and Y;
—X(236)- is selected from the group consisting of no amino acid, G, A, D, E, F, H, I, K, L, M, N, P, Q, R, S, T, V, W and Y;
—X(237)- is selected from the group consisting of G, D, E, F, H, I, K, L, M, N, P, Q, R, S, T, V, W and Y;
—X(238)- is selected from the group consisting of P, D, E, F, G, H, I, K, L, M, N, Q, R, S, T, V, W and Y;
—X(239)- is selected from the group consisting of S, D, E, F, G, H, I, K, L, M, N, P, Q, R, T, V, W and Y;
—X(240)- is selected from the group consisting of V, A, I, M and T;
—X(241)- is selected from the group consisting of F, D, E, L, R, S, W and Y;
—X(243)- is selected from the group consisting of F, E, H, L, Q, R, W and Y;
—X(244)- is selected from the group consisting of P and H;
—X(245)- is selected from the group consisting of P and A;
—X(246)- is selected from the group consisting of K, D, E, H and Y;
—X(247)- is selected from the group consisting of P, G and V;
—X(249)- is selected from the group consisting of D, H, Q and Y;
—X(255)- is selected from the group consisting of R, E and Y;
—X(258)- is selected from the group consisting of E, H, S and Y;
—X(260)- is selected from the group consisting of T, D, E, H and Y;
—X(262)- is selected from the group consisting of V, A, E, F, I and T;
—X(263)- is selected from the group consisting of V, A, I, M and T;
—X(264)- is selected from the group consisting of V, A, D, E, F, G, H, I, K, L, M, N, P, Q, R, S, T, W, and Y;
—X(265)- is selected from the group consisting of D, F, G, H, I, K, L, M, P, Q, R, S, T, V, W and Y;
—X(266)- is selected from the group consisting of V, A, I, M and T;
—X(267)- is selected from the group consisting of S, D, E, F, H, I, K, L, M, N, P, Q, R, V, W and Y;
—X(268)- is selected from the group consisting of H, Q, D, E, F, G, I, K, L, M, P, R, T, V and W;
—X(269)- is selected from the group consisting of E, F, G, H, I, K, L, M, N, P, R, S, T, V, W and Y;
—X(270)- is selected from the group consisting of D, F, G, H, I, L, M, P, Q, R, S, T, W and Y;
—X(271)- is selected from the group consisting of P, A, D, E, F, G, H, I, K, L, M, N, Q, R, S, T, V, W and Y;
—X(272)- is selected from the group consisting of E, D, F, G, H, I, K, L, M, P, R, S, T, V, W and Y;
—X(273)- is selected from the group consisting of V and I;
—X(274)- is selected from the group consisting of Q, K, D, E, F, G, H, I, L, M, N, P, R, T, V, W and Y;
—X(275)- is selected from the group consisting of F, L and W;
—X(276)- is selected from the group consisting of N, K, D, E, F, G, H, I, L, M, P, R, S, T, V, W and Y;
—X(278)- is selected from the group consisting of Y, D, E, G, H, I, K, L, M, N, P, Q, R, S, T, V and W;
—X(280)- is selected from the group consisting of D, G, K, L, P and W;
—X(281)- is selected from the group consisting of G, D, E, K, N, P, Q and Y;
—X(282)- is selected from the group consisting of V, E, G, K, P and Y;
—X(283)- is selected from the group consisting of E, G, H, K, L, P, R and Y;
—X(284)- is selected from the group consisting of V, D, E, L, N, Q, T and Y;
—X(285)- is selected from the group consisting of H, D, E, K, Q, W and Y;
—X(286)- is selected from the group consisting of N, E, G, P and Y;
—X(288)- is selected from the group consisting of K, D, E and Y;
—X(290)- is selected from the group consisting of K, D, H, L, N and W;
—X(291)- is selected from the group consisting of P, D, E, G, H, I, Q and T;
—X(292)- is selected from the group consisting of R, D, E, T and Y;
—X(293)- is selected from the group consisting of E, F, G, H, I, L, M, N, P, R, S, T, V, W and Y;
—X(294)- is selected from the group consisting of E, F, G, H, I, K, L, M, P, R, S, T, V, W and Y;
—X(295)- is selected from the group consisting of Q, D, E, F, G, H, I, M, N, P, R, S, T, V, W and Y;
—X(296)- is selected from the group consisting of F, Y, A, D, E, G, I, K, L, M, N, Q, R, S, T and V;
—X(297)- is selected from the group consisting of N, D, E, F, G, H, I, K, L, M, P, Q, R, S, T, V, W and Y;
—X(298)- is selected from the group consisting of S, E, F, H, I, K, M, Q, R, W and Y;
—X(299)- is selected from the group consisting of T, A, D, E, F, G, H, I, K, L, M, N, P, Q, R, S, V, W and Y;
—X(300)- is selected from the group consisting of F, Y, A, D, E, G, H, K, M, N, P, Q, R, S, T, V and W;
—X(301)- is selected from the group consisting of R, D, E, H and Y;
—X(302)- is selected from the group consisting of V and I;
—X(303)- is selected from the group consisting of V, D, E and Y;
—X(304)- is selected from the group consisting of S, D, H, L, N and T;
—X(305)- is selected from the group consisting of V, E, T and Y;
—X(309)- is selected from the group consisting of V and L;
—X(313)- is selected from the group consisting of W and F;
—X(317)- is selected from the group consisting of K, E and Q;
—X(318)- is selected from the group consisting of E, H, L, Q, R and Y;
—X(320)- is selected from the group consisting of K, D, F, G, H, I, L, N, P, S, T, V, W and Y;
—X(322)- is selected from the group consisting of K, D, F, G, H, I, P, S, T, V, W and Y;
—X(323)- is selected from the group consisting of V and I;
—X(324)- is selected from the group consisting of S, D, F, G, H, I, L, M, P, R, T, V, W and Y;
—X(325)- is selected from the group consisting of N, A, D, E, F, G, H, I, K, L, M, P, Q, R, S, T, V, W and Y;
—X(326)- is selected from the group consisting of K, I, L, P and T;
—X(327)- is selected from the group consisting of G, A, D, E, F, H, I, K, L, M, N, P, R, T, V, W and Y;
—X(328)- is selected from the group consisting of L, A, D, E, F, G, H, I, K, M, N, P, Q, R, S, T, V, W and Y;
—X(329)- is selected from the group consisting of P, D, E, F, G, H, I, K, L, M, N, Q, R, S, T, V, W and Y;
—X(330)- is selected from the group consisting of A, S, E, F, G, H, I, L, M, N, P, R, T, V, W and Y;
—X(331)- is selected from the group consisting of P, S, D, F, H, I, L, M, Q, R, T, V, W and Y;
—X(332)- is selected from the group consisting of I, A, D, E, F, H, K, L, M, N, P, Q, R, S, T, V, W and Y;
—X(333)- is selected from the group consisting of E, F, H, I, L, M, P, T and Y;
—X(334)- is selected from the group consisting of K, F, I, P and T;
—X(335)- is selected from the group consisting of T, D, F, G, H, I, L, M, N, P, R, S, V, W and Y;
—X(336)- is selected from the group consisting of I, E, K and Y;
—X(337)- is selected from the group consisting of S, E, H and N;
—X(339)- is selected from the group consisting of T and A;
—X(355)- is selected from the group consisting of R and Q;
—X(356)- is selected from the group consisting of E and D;
—X(358)- is selected from the group consisting of M and L;
—X(384)- is selected from the group consisting of N and S;
—X(392)- is selected from the group consisting of K and N;
—X(397)- is selected from the group consisting of M and V;
—X(409)- is selected from the group consisting of K and R;
—X(419)- is selected from the group consisting of Q and E;
—X(422)- is selected from the group consisting of V and I;
—X(435)- is selected from the group consisting of H and R;
—X(436)- is selected from the group consisting of Y and F; and
—X(445)- is selected from the group consisting of P and L.

In one variation, a first modification is selected from among S228P, S228R, substitution of the sequence

(SEQ ID NO: 111)

RCPEPKSCDTPPPCPRCPEPKSCDTPPPCPRCPEPKSCDTPPPCPR

at 228, E233P, F234L, F234V, L235A, G236-, Q268H, Q274K, N276K, F296Y, Y300F, L309V, G327A, S330A, S331P, A339T, Q355R, E356D, M358L, N384S, K392N, V397M, R409K, E419Q, V422I, H435R, Y436F, and L445P. In a further variation, a modification is selected from among, 227E, 227G, 227K, 227Y, 228E, 228G, 228K, 228Y, 230A, 230E, 230G, 230Y, 231E, 231G, 231K, 231P, 231Y, 232E, 232G, 232K, 232Y, 233A, 233D, 233F, 233G, 233H, 233I, 233K, 233L, 233M, 233N, 233Q, 233R, 233S, 233T, 233V, 233W, 233Y, 234D, 234E, 234F, 234G, 234H, 234I, 234K, 234M, 234N, 234P, 234Q, 234R, 234S, 234T, 234W, 234Y, 235D, 235F, 235G, 235H, 235I, 235K, 235M, 235N, 235P, 235Q, 235R, 235S, 235T, 235V, 235W, 235Y, 236A, 236D, 236E, 236F, 236H, 236I, 236K, 236L, 236M, 236N, 236P, 236Q, 236R, 236S, 236T, 236V, 236W, 236Y, 237D, 237E, 237F, 237H, 237I, 237K, 237L, 237M, 237N, 237P, 237Q, 237R, 237S, 237T, 237V, 237W, 237Y, 238D, 238E, 238F, 238G, 238H, 238I, 238K, 238L, 238M, 238N, 238Q, 238R, 238S, 238T, 238V, 238W, 238Y, 239D, 239E, 239F, 239G, 239H, 239I, 239K, 239L, 239M, 239N, 239P, 239Q, 239R, 239T, 239V, 239W, 239Y, 240A, 240I, 240M, 240T, 241D, 241E, 241L, 241R, 241S, 241W, 241Y, 243E, 243H, 243L, 243Q, 243R, 243W, 243Y, 244H, 245A, 246D, 246E, 246H, 246Y, 247G, 247V, 249H, 249Q, 249Y, 255E, 255Y, 258H, 258S, 258Y, 260D, 260E, 260H, 260Y, 262A, 262E, 262F, 262I, 262T, 263A, 263I, 263M, 263T, 264A, 264D, 264E, 264F, 264G, 264H, 264I, 264K, 264L, 264M, 264N, 264P, 264Q, 264R, 264S, 264T, 264W, 264Y, 265F, 265G, 265H, 265I, 265K, 265L, 265M, 265P, 265Q, 265R, 265S, 265T, 265V, 265W, 265Y, 266A, 266I, 266M, 266T, 267D, 267E, 267F, 267H, 267I, 267K, 267L, 267M, 267N, 267P, 267Q, 267R, 267V, 267W, 267Y, 268D, 268E, 268F, 268G, 268I, 268K, 268L, 268M, 268P, 268R, 268T, 268V, 268W, 269F, 269G, 269H, 269I, 269K, 269L, 269M, 269N, 269P, 269R, 269S, 269T, 269V, 269W, 269Y, 270F, 270G, 270H, 270I, 270L, 270M, 270P, 270Q, 270R, 270S, 270T, 270W, 270Y, 271A, 271D, 271E, 271F, 271G, 271H, 271I, 271K, 271L, 271M, 271N, 271Q, 271R, 271S, 271T, 271V, 271W, 271Y, 272D, 272F, 272G, 272H, 272I, 272K, 272L, 272M, 272P, 272R, 272S, 272T, 272V, 272W, 272Y, 273I, 274D, 274E, 274F, 274G, 274H, 274I, 274L, 274M, 274N, 274P, 274R, 274T, 274V, 274W, 274Y, 275L, 275W, 276D, 276E, 276F, 276G, 276H, 276I, 276L, 276M, 276P, 276R, 276S, 276T, 276V, 276W, 276Y, 278D, 278E, 278G, 278H, 278I, 278K, 278L, 278M, 278N, 278P, 278Q, 278R, 278S, 278T, 278V, 278W, 280G, 280K, 280L, 280P, 280W, 281D, 281E, 281K, 281N, 281P, 281Q, 281Y, 282E, 282G, 282K, 282P, 282Y, 283G, 283H, 283K, 283L, 283P, 283R, 283Y, 284D, 284E, 284L, 284N, 284Q, 284T, 284Y, 285D, 285E, 285K, 285Q, 285W, 285Y, 286E, 286G, 286P, 286Y, 288D, 288E, 288Y, 290D, 290H, 290L, 290N, 290W, 291D, 291E, 291G, 291H, 291I, 291Q, 291T, 292D, 292E, 292T, 292Y, 293F, 293G, 293H, 293I, 293L, 293M, 293N, 293P, 293R, 293S, 293T, 293V, 293W, 293Y, 294F, 294G, 294H, 294I, 294K, 294L, 294M, 294P, 294R, 294S, 294T, 294V, 294W, 294Y, 295D, 295E, 295F, 295G, 295H, 295I, 295M, 295N, 295P, 295R, 295S, 295T, 295V, 295W, 295Y, 296A, 296D, 296E, 296G, 296I, 296K, 296L, 296M, 296N, 296Q, 296R, 296S, 296T, 296V, 297D, 297E, 297F, 297G, 297H, 297I, 297K, 297L, 297M, 297P, 297Q, 297R, 297S, 297T, 297V, 297W, 297Y, 298E, 298F, 298H, 298I, 298K, 298M, 298Q, 298R, 298W, 298Y, 299A, 299D, 299E, 299F, 299G, 299H, 299I, 299K, 299L, 299M, 299N, 299P, 299Q, 299R, 299S, 299V, 299W, 299Y, 300A, 300D, 300E, 300G, 300H, 300K, 300M, 300N, 300P, 300Q, 300R, 300S, 300T, 300V, 300W, 301D, 301E, 301H, 301Y, 302I, 303D, 303E, 303Y, 304D, 304H, 304L, 304N, 304T, 305E, 305T, 305Y, 313F, 317E, 317Q, 318H, 318L, 318Q, 318R, 318Y, 320D, 320F, 320G, 320H, 320I, 320L, 320N, 320P, 320S, 320T, 320V, 320W, 320Y, 322D, 322F, 322G, 322H, 322I, 322P, 322S, 322T, 322V, 322W, 322Y, 323I, 324D, 324F, 324G, 324H, 324I, 324L, 324M, 324P, 324R, 324T, 324V, 324W, 324Y, 325A, 325D, 325E, 325F, 325G, 325H, 325I, 325K, 325L, 325M, 325P, 325Q, 325R, 325S, 325T, 325V, 325W, 325Y, 326I, 326L, 326P, 326T, 327D, 327E, 327F, 327H, 327I, 327K, 327L, 327M, 327N, 327P, 327R, 327T, 327V, 327W, 327Y, 328A, 328D, 328E, 328F, 328G, 328H, 328I, 328K, 328M, 328N, 328P, 328Q, 328R, 328S, 328T, 328V, 328W, 328Y, 329D, 329E, 329F, 329G, 329H, 329I, 329K, 329L, 329M, 329N, 329Q, 329R, 329S, 329T, 329V, 329W, 329Y, 330E, 330F, 330G, 330H, 330I, 330L, 330M, 330N, 330P, 330R, 330T, 330V, 330W, 330Y, 331D, 331F, 331H, 331I, 331L, 331M, 331Q, 331R, 331T, 331V, 331W, 331Y, 332A, 332D, 332E, 332F, 332H, 332K, 332L, 332M, 332N, 332P, 332Q, 332R, 332S, 332T, 332V, 332W, 332Y, 333F, 333H, 333I, 333L, 333M, 333P, 333T, 333Y, 334F, 334I, 334P, 334T, 335D, 335F, 335G, 335H, 335I, 335L, 335M, 335N, 335P, 335R, 335S, 335V, 335W, 335Y, 336E, 336K, 336Y, 337E, 337H, and 337N.

In a further aspect, the present application is directed to an IgG4 variant amino acid sequence including at least two modifications as compared to SEQ ID NO:13. In certain variations, a first modification is selected from among Q268H, Q274K, N276K, F296Y, Y300F, L309V, G327A, S330A, S331P, A339T, Q355R, E356D, M358L, N384S, K392N, V397M, R409K, E419Q, V422I, H435R, Y436F, and L445P. In further variations, a second modification is selected from among 237D, 237E, 237F, 237H, 237I, 237K, 237L, 237M, 237N, 237P, 237Q, 237R, 237S, 237T, 237V, 237W, 237Y, 238D, 238E, 238F, 238G, 238H, 238I, 238K, 238L, 238M, 238N, 238Q, 238R, 238S, 238T, 238V, 238W, 238Y, 239D, 239E, 239F, 239G, 239H, 239I, 239K, 239L, 239M, 239N, 239P, 239Q, 239R, 239T, 239V, 239W, 239Y, 240A, 240I, 240M, 240T, 241D, 241E, 241L, 241R, 241S, 241W, 241Y, 243E, 243H, 243L, 243Q, 243R, 243W, 243Y, 244H, 245A, 246D, 246E, 246H, 246Y, 247G, 247V, 249H, 249Q, 249Y, 255E, 255Y, 258H, 258S, 258Y, 260D, 260E, 260H, 260Y, 262A, 262E, 262F, 262I, 262T, 263A, 263I, 263M, 263T, 264A, 264D, 264E, 264F, 264G, 264H, 264I, 264K, 264L, 264M, 264N, 264P, 264Q, 264R, 264S, 264T, 264W, 264Y, 265F, 265G, 265H, 265I, 265K, 265L, 265M, 265P, 265Q, 265R, 265S, 265T, 265V, 265W, 265Y, 266A, 266I, 266M, 266T, 267D, 267E, 267F, 267H, 267I, 267K, 267L, 267M, 267N, 267P, 267Q, 267R, 267V, 267W, 267Y, 268D, 268E, 268F, 268G, 268I, 268K, 268L, 268M, 268P, 268R, 268T, 268V, 268W, 269F, 269G, 269H, 269I, 269K, 269L, 269M, 269N, 269P, 269R, 269S, 269T, 269V, 269W, 269Y, 270F, 270G, 270H, 270I, 270L, 270M, 270P, 270Q, 270R, 270S, 270T, 270W, 270Y, 271A, 271D, 271E, 271F, 271G, 271H, 271I, 271K, 271L, 271M, 271N, 271Q, 271R, 271S, 271T, 271V, 271W, 271Y, 272D, 272F, 272G, 272H, 272I, 272K, 272L, 272M, 272P, 272R, 272S, 272T, 272V, 272W, 272Y, 273I, 274D, 274E, 274F, 274G, 274H, 274I, 274L, 274M, 274N, 274P, 274R, 274T, 274V, 274W, 274Y, 275L, 275W, 276D, 276E, 276F, 276G, 276H, 276I, 276L, 276M, 276P, 276R, 276S, 276T, 276V, 276W, 276Y, 278D, 278E, 278G, 278H, 278I, 278K, 278L, 278M, 278N, 278P, 278Q, 278R, 278S, 278T, 278V, 278W, 280G, 280K, 280L, 280P, 280W, 281D, 281E, 281K, 281N, 281P, 281Q, 281Y, 282E, 282G, 282K, 282P, 282Y, 283G, 283H, 283K, 283L, 283P, 283R, 283Y, 284D, 284E, 284L, 284N, 284Q, 284T, 284Y, 285D, 285E, 285K, 285Q, 285W, 285Y, 286E, 286G, 286P, 286Y, 288D, 288E, 288Y, 290D, 290H, 290L, 290N, 290W, 291D, 291E, 291G, 291H, 291I, 291Q, 291T, 292D, 292E, 292T, 292Y, 293F, 293G, 293H, 293I, 293L, 293M, 293N, 293P, 293R, 293S, 293T, 293V, 293W, 293Y, 294F, 294G, 294H, 294I, 294K, 294L, 294M, 294P, 294R, 294S, 294T, 294V, 294W, 294Y, 295D, 295E, 295F, 295G, 295H, 295I, 295M, 295N, 295P, 295R, 295S, 295T, 295V, 295W, 295Y, 296A, 296D, 296E, 296G, 296I, 296K, 296L, 296M, 296N, 296Q, 296R, 296S, 296T, 296V, 297D, 297E, 297F, 297G, 297H, 297I, 297K, 297L, 297M, 297P, 297Q, 297R, 297S, 297T, 297V, 297W, 297Y, 298E, 298F, 298H, 298I, 298K, 298M, 298Q, 298R, 298W, 298Y, 299A, 299D, 299E, 299F, 299G, 299H, 299I, 299K, 299L, 299M, 299N, 299P, 299Q, 299R, 299S, 299V, 299W, 299Y, 300A, 300D, 300E, 300G, 300H, 300K, 300M, 300N, 300P, 300Q, 300R, 300S, 300T, 300V, 300W, 301D, 301E, 301H, 301Y, 302I, 303D, 303E, 303Y, 304D, 304H, 304L, 304N, 304T, 305E, 305T, 305Y, 313F, 317E, 317Q, 318H, 318L, 318Q, 318R, 318Y, 320D, 320F, 320G, 320H, 320I, 320L, 320N, 320P, 320S, 320T, 320V, 320W, 320Y, 322D, 322F, 322G, 322H, 322I, 322P, 322S, 322T, 322V, 322W, 322Y, 323I, 324D, 324F, 324G, 324H, 324I, 324L, 324M, 324P, 324R, 324T, 324V, 324W, 324Y, 325A, 325D, 325E, 325F, 325G, 325H, 325I, 325K, 325L, 325M, 325P, 325Q, 325R, 325S, 325T, 325V, 325W, 325Y, 326I, 326L, 326P, 326T, 327D, 327E, 327F, 327H, 327I, 327K, 327L, 327M, 327N, 327P, 327R, 327T, 327V, 327W, 327Y, 328A, 328D, 328E, 328F, 328G, 328H, 328I, 328K, 328M, 328N, 328P, 328Q, 328R, 328S, 328T, 328V, 328W, 328Y, 329D, 329E, 329F, 329G, 329H, 329I, 329K, 329L, 329M, 329N, 329Q, 329R, 329S, 329T, 329V, 329W, 329Y, 330E, 330F, 330G, 330H, 330I, 330L, 330M, 330N, 330P, 330R, 330T, 330V, 330W, 330Y, 331D, 331F, 331H, 331I, 331L, 331M, 331Q, 331R, 331T, 331V, 331W, 331Y, 332A, 332D, 332E, 332F, 332H, 332K, 332L, 332M, 332N, 332P, 332Q, 332R, 332S, 332T, 332V, 332W, 332Y, 333F, 333H, 333I, 333L, 333M, 333P, 333T, 333Y, 334F, 334I, 334P, 334T, 335D, 335F, 335G, 335H, 335I, 335L, 335M, 335N, 335P, 335R, 335S, 335V, 335W, 335Y, 336E, 336K, 336Y, 337E, 337H, and 337N.

In another aspect, the present application is directed to an IgG4 variant including an amino acid sequence having the formula:

(SED ID NO: 108)

C-X(227)-X(228)-CPAP-X(233)-X(234)-X(235)-X(236)-
X(237)-P-X(239)-X(240)-FLFPP-X(246)-PKDTLMIS-
X(255)-TP-X(258)-V-X(260)-CVV-X(264)-DV-X(267)-
X(268)-ED-X(271)-X(272)-V-X(274)-F-X(276)-W-
X(278)-VD-X(281)-V-X(283)-X(284)-HNAKT-X(290)-
PR-X(293)-E-X(295)-X(296)-NST-X(300)-RVV-X(304)-
VLTV-X(309)-HQDWLNGKEYKCKV-X(324)-N-X(326)-
X(327)-X(328)-P-X(330)-X(331)-X(332)-X(333)-
X(334)-TISK-X(339)-KGQPREPQVYTLPPS-X(355)-
X(356)-E-X(358)-TKNQVSLTCLVKGFYPSDIAVEWES-
X(384)-GQPENNY-X(392)-TTPP-X(397)-LDSDGSFFLYS-
X(409)-LTVDKSRWQ-X(419)-GN-X(422)-FSCSVMHEALHN-
X(435)-X(436)-TQKSLSLS-X(445)-GK,

wherein
—X(227)- is selected from the group consisting of P and G;
—X(228)- is selected from the group consisting of P, R, S, and the sequence

(SEQ ID NO: 111)

RCPEPKSCDTPPPCPRCPEPKSCDTPPPCPRCPEPKSCDTPPPCPR;

—X(233)- is selected from the group consisting of P and E;
—X(234)- is selected from the group consisting of V, L, F, Y and I;
—X(235)- is selected from the group consisting of A, L, Y, I and D;
—X(236)- is selected from the group consisting of no amino acid, G, S and A;
—X(237)- is selected from the group consisting of G and D;
—X(239)- is selected from the group consisting of S, D, E, N, Q and T;
—X(240)- is selected from the group consisting of V, I and M;
—X(246)- is selected from the group consisting of K, H and Y;
—X(255)- is selected from the group consisting of R and Y;
—X(258)- is selected from the group consisting of E, H and Y;
—X(260)- is selected from the group consisting of T and H;
—X(264)- is selected from the group consisting of V, I, T and Y;
—X(267)- is selected from the group consisting of S, D and E;
—X(268)- is selected from the group consisting of H, Q, D and E;
—X(271)- is selected from the group consisting of P and G;
—X(272)- is selected from the group consisting of E, Y, H, R and I;
—X(274)- is selected from the group consisting of Q, K and E;
—X(276)- is selected from the group consisting of N and K;
—X(278)- is selected from the group consisting of Y and T;
—X(281)- is selected from the group consisting of G, D and E;
—X(283)- is selected from the group consisting of E, L and H;
—X(284)- is selected from the group consisting of V, E and D;
—X(290)- is selected from the group consisting of K and N;
—X(293)- is selected from the group consisting of E and R;
—X(295)- is selected from the group consisting of Q and E;
—X(296)- is selected from the group consisting of F and Y;
—X(300)- is selected from the group consisting of F and Y;
—X(304)- is selected from the group consisting of S and T;
—X(309)- is selected from the group consisting of V and L;
—X(324)- is selected from the group consisting of S, G and I;
—X(326)- is selected from the group consisting of K and T;
—X(327)- is selected from the group consisting of G, A and D;
—X(328)- is selected from the group consisting of L, A, F, I and T;
—X(330)- is selected from the group consisting of A, S, L, Y and I;
—X(331)- is selected from the group consisting of P and S;
—X(332)- is selected from the group consisting of I, D, E, N, Q and T;
—X(333)- is selected from the group consisting of E and Y;
—X(334)- is selected from the group consisting of K, F, I and T;
—X(339)- is selected from the group consisting of T and A;
—X(355)- is selected from the group consisting of R and Q;
—X(356)- is selected from the group consisting of E and D;
—X(358)- is selected from the group consisting of M and L;
—X(384)- is selected from the group consisting of N and S;
—X(392)- is selected from the group consisting of K and N;
—X(397)- is selected from the group consisting of M and V;
—X(409)- is selected from the group consisting of K and R;
—X(419)- is selected from the group consisting of Q and E;
—X(422)- is selected from the group consisting of V and I;
—X(435)- is selected from the group consisting of H and R;
—X(436)- is selected from the group consisting of Y and F; and
—X(445)- is selected from the group consisting of P and L.

In one variation, a first modification is selected from among S228P, S228R, substitution of the sequence

(SEQ ID NO: 111)

RCPEPKSCDTPPPCPRCPEPKSCDTPPPCPRCPEPKSCDTPPPCPR

at 228, E233P, F234L, F234V, L235A, G236-, Q268H, Q274K, N276K, F296Y, Y300F, L309V, G327A, S330A, S331P, A339T, Q355R, E356D, M358L, N384S, K392N, V397M, R409K, E419Q, V422I, H435R, Y436F, and L445P. In a further variation, a second modification is selected from among 227G, 234Y, 234I, 235Y, 235I, 235D, 236S, 236A, 237D, 239D, 239E, 239N, 239Q, 239T, 240I, 240M, 246H, 246Y, 255Y, 258H, 258Y, 260H, 264I, 264T, 264Y, 267D, 267E, 268D, 268E, 271G, 272Y, 272H, 272R, 272I, 274E, 278T, 281D, 281E, 283L, 283H, 284E, 284D, 290N, 293R, 295E, 304T, 324G, 324I, 326T, 327D, 328A, 328F, 328I, 328T, 330L, 330Y, 330I, 332D, 332E, 332N, 332Q, 332T, 333Y, 334F, 334I, and 334T. Alternatively, the modifications can be selected from among those beginning at position 230 until the C terminus.

In another aspect, the present application is directed to an IgG4 variant including an amino acid sequence having the formula:

(SED ID NO: 109)

ASTKGPSVFPLAPCSRSTSESTAALGCLVKDYFPEPVTVSWNSGALTSGV
HTFPAVLQSSGLY SLSSVVTVPSSSLGTKTYTCNVDHKPSNTKVDKRVE
SKYG-X(221)-X(222)-X(223)-X(224)-X(225)-C-X(227)-
X(228)-C-X(230)-X(231)-X(232)-X(233)-X(234)-
X(235)-X(236)-X(237)-X(238)-X(239)-X(240)-X(241)-
L-X(243)-X(244)-X(245)-X(246)-X(247)-K-X(249)-
TLMIS-X(255)-TP-X(258)-V-X(260)-C-X(262)-X(263)-
X(264)-X(265)-X(266)-X(267)-X(268)-X(269)-X(270)-
X(271)-X(272)-X(273)-X(274)-X(275)-X(276)-W-
X(278)-V-X(280)-X(281)-X(282)-X(283)-X(284)-
X(285)-X(286)-A-X(288)-T-X(290)-X(291)-X(292)-
X(293)-X(294)-X(295)-X(296)-X(297)-X(298)-X(299)-
X(300)-X(301)-X(302)-X(303)-X(304)-X(305)-LTVLHQD-
X(313)-LNG-X(317)-X(318)-Y-X(320)-C-X(322)-X(323)-
X(324)-X(325)-X(326)-X(327)-X(328)-X(329)-X(330)-
X(331)-X(332)-X(333)-X(334)-X(335)-X(336)-X(337)-
KAKGQPREPQVYTLPPSQEEMTKNQVSLTCLVKGFYPSDIAVEWESNGQ
PENNYKTTPPVLD SDGSFFLYSRLTVDKSRWQEGNVFSCSVMHEALHN
HYTQKSLSLSLGK-

wherein
—X(221)- is selected from the group consisting of no amino acid, K and Y;
—X(222)- is selected from the group consisting of no amino acid, E and Y;
—X(223)- is selected from the group consisting of no amino acid, E and K;
—X(224)- is selected from the group consisting of P and Y;
—X(225)- is selected from the group consisting of P, E, K and W;
—X(227)- is selected from the group consisting of P, E, G, K and Y;
—X(228)- is selected from the group consisting of S, E, G, K and Y;
—X(230)- is selected from the group consisting of P, A, E, G and Y;
—X(231)- is selected from the group consisting of A, E, G, K, P and Y;
—X(232)- is selected from the group consisting of P, E, G, K and Y;
—X(233)- is selected from the group consisting of E, A, D, F, G, H, I, K, L, M, N, Q, R, S, T, V, W and Y;
—X(234)- is selected from the group consisting of F, D, E, F, G, H, I, K, M, N, P, Q, R, S, T, W and Y;
—X(235)- is selected from the group consisting of L, D, F, G, H, I, K, M, N, P, Q, R, S, T, V, W and Y;
—X(236)- is selected from the group consisting of G, A, D, E, F, H, I, K, L, M, N, P, Q, R, S, T, V, W and Y;
—X(237)- is selected from the group consisting of G, D, E, F, H, I, K, L, M, N, P, Q, R, S, T, V, W and Y;
—X(238)- is selected from the group consisting of P, D, E, F, G, H, I, K, L, M, N, Q, R, S, T, V, W and Y;
—X(239)- is selected from the group consisting of S, D, E, F, G, H, I, K, L, M, N, P, Q, R, T, V, W and Y;
—X(240)- is selected from the group consisting of V, A, I, M and T;
—X(241)- is selected from the group consisting of F, D, E, L, R, S, W and Y;
—X(243)- is selected from the group consisting of F, E, H, L, Q, R, W and Y;
—X(244)- is selected from the group consisting of P and H;
—X(245)- is selected from the group consisting of P and A;
—X(246)- is selected from the group consisting of K, D, E, H and Y;
—X(247)- is selected from the group consisting of P, G and V;
—X(249)- is selected from the group consisting of D, H, Q and Y;
—X(255)- is selected from the group consisting of R, E and Y;
—X(258)- is selected from the group consisting of E, H, S and Y;
—X(260)- is selected from the group consisting of T, D, E, H and Y;
—X(262)- is selected from the group consisting of V, A, E, F, I and T;
—X(263)- is selected from the group consisting of V, A, I, M and T;
—X(264)- is selected from the group consisting of V, A, D, E, F, G, H, I, K, L, M, N, P, Q, R, S, T, W and Y;
—X(265)- is selected from the group consisting of D, F, G, H, I, K, L, M, P, Q, R, S, T, V, W and Y;
—X(266)- is selected from the group consisting of V, A, I, M and T;
—X(267)- is selected from the group consisting of S, D, E, F, H, I, K, L, M, N, P, Q, R, V, W and Y;
—X(268)- is selected from the group consisting of Q, D, E, F, G, I, K, L, M, P, R, T, V and W;
—X(269)- is selected from the group consisting of E, F, G, H, I, K, L, M, N, P, R, S, T, V, W and Y;
—X(270)- is selected from the group consisting of D, F, G, H, I, L, M, P, Q, R, S, T, W and Y;
—X(271)- is selected from the group consisting of P, A, D, E, F, G, H, I, K, L, M, N, Q, R, S, T, V, W and Y;
—X(272)- is selected from the group consisting of E, D, F, G, H, I, K, L, M, P, R, S, T, V, W and Y;
—X(273)- is selected from the group consisting of V and I;
—X(274)- is selected from the group consisting of Q, D, E, F, G, H, I, L, M, N, P, R, T, V, W and Y;
—X(275)- is selected from the group consisting of F, L, W;
—X(276)- is selected from the group consisting of N, D, E, F, G, H, I, L, M, P, R, S, T, V, W and Y;
—X(278)- is selected from the group consisting of Y, D, E, G, H, I, K, L, M, N, P, Q, R, S, T, V and W;
—X(280)- is selected from the group consisting of D, G, K, L, P and W;
—X(281)- is selected from the group consisting of G, D, E, K, N, P, Q and Y;
—X(282)- is selected from the group consisting of V, E, G, K, P and Y;
—X(283)- is selected from the group consisting of E, G, H, K, L, P, R and Y;
—X(284)- is selected from the group consisting of V, D, E, L, N, Q, T and Y;
—X(285)- is selected from the group consisting of H, D, E, K, Q, W and Y;
—X(286)- is selected from the group consisting of N, E, G, P and Y;
—X(288)- is selected from the group consisting of K, D, E and Y;
—X(290)- is selected from the group consisting of K, D, H, L, N and W;
—X(291)- is selected from the group consisting of P, D, E, G, H, I, Q and T;
—X(292)- is selected from the group consisting of R, D, E, T and Y;
—X(293)- is selected from the group consisting of E, F, G, H, I, L, M, N, P, R, S, T, V, W and Y;
—X(294)- is selected from the group consisting of E, F, G, H, I, K, L, M, P, R, S, T, V, W and Y;
—X(295)- is selected from the group consisting of Q, D, E, F, G, H, I, M, N, P, R, S, T, V, W and Y;
—X(296)- is selected from the group consisting of F, A, D, E, G, I, K, L, M, N, Q, R, S, T and V;
—X(297)- is selected from the group consisting of N, D, E, F, G, H, I, K, L, M, P, Q, R, S, T, V, W and Y;
—X(298)- is selected from the group consisting of S, E, F, H, I, K, M, Q, R, W and Y;
—X(299)- is selected from the group consisting of T, A, D, E, F, G, H, I, K, L, M, N, P, Q, R, S, V, W and Y;
—X(300)- is selected from the group consisting of Y, A, D, E, G, H, K, M, N, P, Q, R, S, T, V and W;
—X(301)- is selected from the group consisting of R, D, E, H and Y;
—X(302)- is selected from the group consisting of V and I;
—X(303)- is selected from the group consisting of V, D, E and Y;
—X(304)- is selected from the group consisting of S, D, H, L, N and T;
—X(305)- is selected from the group consisting of V, E, T and Y;
—X(313)- is selected from the group consisting of W and F;
—X(317)- is selected from the group consisting of K, E and Q;
—X(318)- is selected from the group consisting of E, H, L, Q, R and Y;
—X(320)- is selected from the group consisting of K, D, F, G, H, I, L, N, P, S, T, V, W and Y;
—X(322)- is selected from the group consisting of K, D, F, G, H, I, P, S, T, V, W and Y;
—X(323)- is selected from the group consisting of V and I;
—X(324)- is selected from the group consisting of S, D, F, G, H, I, L, M, P, R, T, V, W and Y;
—X(325)- is selected from the group consisting of N, A, D, E, F, G, H, I, K, L, M, P, Q, R, S, T, V, W and Y;
—X(326)- is selected from the group consisting of K, I, L, P and T;
—X(327)- is selected from the group consisting of G, D, E, F, H, I, K, L, M, N, P, R, T, V, W and Y;
—X(328)- is selected from the group consisting of L, A, D, E, F, G, H, I, K, M, N, P, Q, R, S, T, V, W and Y;
—X(329)- is selected from the group consisting of P, D, E, F, G, H, I, K, L, M, N, Q, R, S, T, V, W and Y;
—X(330)- is selected from the group consisting of S, E, F, G, H, I, L, M, N, P, R, T, V, W and Y;
—X(331)- is selected from the group consisting of S, D, F, H, I, L, M, Q, R, T, V, W and Y;
—X(332)- is selected from the group consisting of I, A, D, E, F, H, K, L, M, N, P, Q, R, S, T, V, W and Y;
—X(333)- is selected from the group consisting of E, F, H, I, L, M, P, T and Y;
—X(334)- is selected from the group consisting of K, F, I, P and T;
—X(335)- is selected from the group consisting of T, D, F, G, H, I, L, M, N, P, R, S, V, W and Y;
—X(336)- is selected from the group consisting of I, E, K and Y; and
—X(337)- is selected from the group consisting of S, E, H and N.

In another aspect, the present application is directed to an IgG4 variant including an amino acid sequence having the formula:

(SED ID NO: 110)

ASTKGPSVFPLAPCSRSTSESTAALGCLVKDYFPEPVTVSWNSGALTSGV
HTFPAVLQSSGLYSLSSVVTVPSSSLGTKTYTCNVDHKPSNTKVDKRVES
KYG-X(221)-PPC-X(227)-SCPAPE-X(234)-X(235)-X(236)-
X(237)-P-X(239)-X(240)-FLFPP-X(246)-PKDTLMIS-
X(255)-TP-X(258)-V-X(260)-CVV-X(264)-DV-X(267)-
X(268)-ED-X(271)-X(272)-V-X(274)-FNW-X(278)-VD-
X(281)-V-X(283)-X(284)-HNAKT-X(290)-PR-X(293)-E-
X(295)-FNSTYRVV-X(304)-VLTVLHQDWLNGKEYKCKV-X(324)-
N-X(326)-X(327)-X(328)-P-X(330)-S-X(332)-X(333)-
X(334)-TISKAKGQPREPQVYTLPPSQEEMTKNQVSLTCLVKGFYPSD
IAVEWESNGQPENNYKTTPPVLDSDGSFFLYSRLT VDKSRWQEGNVFS
CSVMHEALHNHYTQKSLSLSLGK

wherein
—X(221)- is selected from the group consisting of no amino acid and K;
—X(227)- is selected from the group consisting of P and G;
—X(234)- is selected from the group consisting of F, Y and I;
—X(235)- is selected from the group consisting of L, Y, I and D;
—X(236)- is selected from the group consisting of G, S and A;
—X(237)- is selected from the group consisting of G and D;
—X(239)- is selected from the group consisting of S, D, E, N, Q and T;
—X(240)- is selected from the group consisting of V, I and M;
—X(246)- is selected from the group consisting of K, H and Y;
—X(255)- is selected from the group consisting of R and Y;
—X(258)- is selected from the group consisting of E, H and Y;
—X(260)- is selected from the group consisting of T and H;
—X(264)- is selected from the group consisting of V, I, T and Y;
—X(267)- is selected from the group consisting of S, D and E;
—X(268)- is selected from the group consisting of Q, D and E;
—X(271)- is selected from the group consisting of P and G;
—X(272)- is selected from the group consisting of E, Y, H, R and I;
—X(274)- is selected from the group consisting of Q and E;
—X(278)- is selected from the group consisting of Y and T;
—X(281)- is selected from the group consisting of G, D and E;
—X(283)- is selected from the group consisting of E, L and H;
—X(284)- is selected from the group consisting of V, E and D;
—X(290)- is selected from the group consisting of K and N;
—X(293)- is selected from the group consisting of E and R;
—X(295)- is selected from the group consisting of Q and E;
—X(304)- is selected from the group consisting of S and T;
—X(324)- is selected from the group consisting of S, G and I;
—X(326)- is selected from the group consisting of K and T;
—X(327)- is selected from the group consisting of G and D;
—X(328)- is selected from the group consisting of L, A, F, I and T;
—X(330)- is selected from the group consisting of S, L, Y and I;
—X(332)- is selected from the group consisting of I, D, E, N, Q and T;
—X(333)- is selected from the group consisting of E and Y; and
—X(334)- is selected from the group consisting of K, F, I and T.

In certain variations, the variant differs from SEQ ID NO:13 by at least one amino acid.

Variations in which modifications are in 2, 3, or 4 different domains, the domains can be selected from among, for example, all IgG domains, only IgG heavy chain domains, and only hinge-CH2-CH3 domains. Alternatively, the domains can be limited to include only Fc region, or only CH2-CH3 domains.

The IgG2, IgG3, or IgG4 variants can improve binding to one or more FcγR, or enhance effector function as compared to a polypeptide having the amino acid sequence of SEQ ID NO:11, SEQ ID NO:12, or SEQ ID NO:13. In certain variations, FcγR is selected from the group consisting of human FcγRI, FcγRIIa, FcγRIIb, FcγRIIc, and FcγRIIIa. In other variations, the additionally reduces binding to human FcγRIIb. Exemplary effector function that is enhanced can be ADCC, ADCP, and CDC.

The present application is also directed to sequence including the variants described herein identified by sequence identification number.

In a preferred embodiment of the invention, the Fc variant alters binding to one or more FcγRs. In one aspect, said Fc variant reduces affinity to a human FcγR. In another aspect, said Fc variant improves affinity to a human FcγR.

The present invention provides novel Fc polypeptides, including antibodies, Fc fusions, isolated Fc, and Fc fragments, that comprise the Fc variants disclosed herein. The novel Fc polypeptides may find use in a therapeutic product. In certain embodiments, the Fc polypeptides of the invention are antibodies.

In one aspect of the invention, the Fc variant of the invention is an antibody having a IgG1, IgG2, IgG3, IgG4, or IgG1/IgG2 scaffold.

The present invention provides isolated nucleic acids encoding the Fc variants described herein. The present invention provides vectors comprising the nucleic acids, optionally, operably linked to control sequences. The present invention provides host cells containing the vectors, and methods for producing and optionally recovering the Fc variants.

The present invention provides compositions comprising Fc polypeptides that comprise the Fc variants described herein, and a physiologically or pharmaceutically acceptable carrier or diluent.

The present invention contemplates therapeutic and diagnostic uses for Fc polypeptides that comprise the Fc variants disclosed herein. The Fc polypeptides described by the invention may be used to treat a variety of indications, including but not limited to cancers, infectious diseases, autoimmune disorders, an infectious diseases.

### BRIEF DESCRIPTION OF THE DRAWINGS

More particular descriptions of the invention are made by reference to certain exemplary embodiments thereof which are illustrated in the appended Figures. These Figures form a part of the specification. It is to be noted, however, that the appended Figures illustrate exemplary embodiments of the invention and therefore are not to be considered limiting in their scope.

FIG. 1a-1b. Alignment of the amino acid sequences of the human IgG immunoglobulins IgG1, IgG2, IgG3, and IgG4. FIG. 1a provides the sequences of the CH1 (Cy1) and hinge domains, and FIG. 1b provides the sequences of the CH2 (Cy2) and CH3 (Cy3) domains. Positions are numbered according to the EU index of the IgG1 sequence, and differences between IgG1 and the other immunoglobulins IgG2, IgG3, and IgG4 are shown in gray. Allotypic polymorphisms exist at a number of positions, and thus slight differences between the presented sequences and sequences in the prior art may exist. The possible beginnings of the Fc region are labeled, defined herein as either EU position 226 or 230.

FIGS. 2a and 2b. Common haplotypes of the human gamma1 (FIG. 2a) and gamma2 (FIG. 2b) chains.

FIGS. 3a and 3b. FIG. 3a provides an illustration of the hinge region and sites of engineering. Gray indicates the C-terminus of the CH1 domain (left) and N-terminus of the CH2 domain (right). Bold indicates residues in the hinge and N-terminal CH2 domain, 233-238, that interact with FcγRs according to the structure of the human Fc/FcγRIIIb complex (pdb 1E4K, Sondermann et al., 2000, Nature 406:267-273). FIG. 3b shows the structured domains (CH2 and CH3) of the Fc region (pdb 1DN2, DeLano et al., 2000, Science 287:1279-1283). The first residues involved in the structured CH2 region, G237 and P238, are shown as black sticks. The carbohydrate attached at N297 is shown as black lines.

FIG. 4. Library of antibody Fc variants screened for reduced FcγR affinity and effector function. # indicates a deletion of the designated residue, and ̂ indicates an insertion of the designated amino acid after the designated position. A description of insertions and deletions is provided for each variant, and the amino acid sequence from EU positions 230-238 is provided.

FIGS. 5a and 5b. Surface Plasmon Resonance (SPR) (Biaore) sensorgrams for binding of WT and Fc variant anti-Her2 antibodies to human Fc receptors. FIG. 5a shows the binding of anti-Her2 WT IgG1 antibody to human FcγRs FcγRI, H131 and R131 FcγRIIa, FcγRIIb, and V158 and F158 FcγRIIIa. Binding was measured at 5 concentrations of receptor. FIG. 5b shows the sensorgram for the highest receptor concentration for binding of select antibodies and antibody variants to each FcγR.

FIG. 6. Table of affinities for binding of WT IgG and Fc variant antibodies to human FcγRs as determined by Biacore. The equilibrium dissociation constant (K_{D}) for binding of each variant to each FcγR is provided where tested. “NB”=no binding detected; “Weak”=binding observed but not fittable to an accurate K_{D}. A blank cell indicates that the receptor was not tested for that particular variant.

FIG. 7. Affinities for binding of WT and Fc variant antibodies to human FcγRs obtained from the data provided in FIG. 6. The graph is a plot of the log of the K_{A} (K_{A}=1/K_{D} as provided in FIG. 6) for binding of each variant to each of the Fc receptors. I=FcγRI, H IIa=H131 FcγRIIa, R IIa=R131 FcγRIIa, IIb=FcγRIIb, V IIIa=V158 FcγRIIIa, and F IIIc=F158 FcγRIIIa.

FIG. 8. SPR sensorgrams at the highest receptor concentration for binding of WT and Fc variant antibodies to human FcγRI.

FIG. 9. SPR sensorgrams at the highest receptor concentration for binding of WT and Fc variant antibodies to human FcγRI, H131 FcγRIIa, R131 FcγRIIa, FcγRIIb, V158 FcγRIIIa, and F158 FcγRIIIa.

FIG. 10. ADCC assay comparing PBMC ADCC activity of anti-Her2 Fc variant antibodies with that of native IgG isotypes IgG1, IgG2, and IgG4.

FIG. 11. ADCP assay comparing macrophage phagocytosis of anti-CD19 Fc variant antibody with that of native IgG1.

FIG. 12. CDC assay comparing complement activity of anti-CD20 Fc variant antibodies with that of native IgG isotypes IgG1, IgG2, and IgG4.

FIGS. 13a and 13b. Preferred modifications of the invention for reducing FcγR- and/or complement-mediated effector function. FIG. 13a shows positions at which insertions and deletions may be constructed. FIG. 13b shows positions and substitutions that may be combined with the modifications provided in FIG. 13a. However, as outlined herein, FIG. 13 is not meant to be limiting, and any amino acid modification described herein or in the applications incorporated by reference can be combined independently with any other(s).

FIG. 14. Library of antibody Fc variants screened for selective FcγR affinity and optimized effector function. # indicates a deletion of the designated residue, and ̂ indicates an insertion of the designated amino acid after the designated position. A description of insertions and deletions is provided for each variant, and the amino acid sequence from 230-238 is provided.

FIGS. 15a and 15b. FIG. 15a provides the affinities for binding of WT IgG and Fc variant antibodies to human FcγRs as determined by Biacore. The equilibrium dissociation constant (K_{D}) for binding of each variant to each FcγR is provided where tested. “NB”=no binding detected. FIG. 15b provides the activating:inhibitory ratios for two activating receptors, FcγRIIa (H131 and R131 isoforms) and FcγRIIIa (V1158 and F158 isoforms) relative to the inhibitory receptor FcγRIIb. These ratios were calculated by dividing the K_{D} for FcγRIIb by the K_{D} for the activating receptor.

FIG. 16. Affinities for binding of WT and Fc variant antibodies to human FcγRs obtained from SPR data provided in FIG. 15. The graph is a plot of the log of the K_{A} (K_{A}=1/K_{D} as provided in FIG. 15) for binding of each variant to each of the Fc receptors. I=FcγRI, H IIa=H131 FcγRIIa, R IIa=R131 FcγRIIa, IIb=FcγRIIb, V IIIa=V158 FcγRIIIa, and F IIIa=F158 FcγRIIIa. ELLG=P233E/V234L/A235L/̂235G.

FIG. 17. Affinity ratios of WT and Fc variant antibodies for the human FcγRs. Data are provided in FIG. 15b. ELLG=P233E/V234L/A235L/̂235G.

FIGS. 18a and 18b. Preferred modifications of the invention for engineering selectively optimized FcγR affinity. FIG. 18a shows positions at which insertions and deletions may be constructed. FIG. 18b shows positions and substitutions that may be combined with the modifications provided in FIG. 18a.

FIGS. 19a-19f Amino acid sequences of variable light (VL) and heavy (VH) chains used in the present invention, including PRO70769 (FIGS. 19a and 19b), trastuzumab (FIGS. 19c and 19d), and ipilimumab (FIGS. 19e and 19f).

FIGS. 20a-20e. Amino acid sequences of human constant light kappa (FIG. 20a) and heavy (FIGS. 20b-20e) chains used in the present invention.

FIG. 21. Antibody structure and function. Shown is a model of a full length human IgG1 antibody, modeled using a humanized Fab structure from pdb accession code 10E1 (James et al., 1999, J Mol Biol 289:293-301) and a human IgG1 Fc structure from pdb accession code 1DN2 (DeLano et al., 2000, Science 287:1279-1283). The flexible hinge that links the Fab and Fc regions is not shown. IgG1 is a homodimer of heterodimers, made up of two light chains and two heavy chains. The Ig domains that comprise the antibody are labeled, and include VL and CL for the light chain, and Vh, CH1 (Cy1), CH2 (Cy2), and CH3 (Cy3) for the heavy chain. The Fc region is labeled. Binding sites for relevant proteins are labeled, including the antigen binding site in the variable region, and the binding sites for FcγRs, FcRn, C1q, and proteins A and Gin the Fc region.

FIG. 22. The Fc/FcγRIIIb complex structure 1IIS. Fc is shown as a gray ribbon diagram, and FcγRIIIb is shown as a black ribbon. The N297 carbohydrate is shown as black sticks.

FIG. 23. Preferred embodiments of receptor binding profiles that include improvements to, reductions to, or no effect to the binding to various receptors, where such changes may be beneficial in certain contexts.

FIG. 24a-24s. The fold- enhancement or reduction relative to WT for binding of Fc variants to Fc ligands, FcγRI, FcγRIIa, FcγRIIb, FcγRIIc, V158 FcγRIIIa, C1q, and FcRn, as measured by the AlphaScreen. The table presents for each variant the variant number (Variant), the substitution(s) of the variant, the antibody context (Context), the fold affinity relative to WT (Fold) and the confidence (Conf) in the fold affinity for binding to each Fc ligand, and the IIIa:IIb specificity ratio (IIIa:IIb) (see below). Multiple data sets were acquired for many of the variants, and all data for a given variant are grouped together. The context of the antibody indicates which antibodies have been constructed with the particular Fc variant; a=alemtuzumab, t=trastuzumab, r=rituximab, c=cetuximab, and p=PRO70769. The data provided were acquired in the context of the first antibody listed, typically alemtuzumab, although in some cases trastuzumab. An asterix (*) indicates that the data for the given Fc ligand was acquired in the context of trastuzumab. A fold (Fold) above 1 indicates an enhancement in binding affinity, and a fold below 1 indicates a reduction in binding affinity relative to the parent antibody for the given Fc ligand. Confidence values (Conf) correspond to the log confidence levels, provided from the fits of the data to a sigmoidal dose response curve. As is known in the art, a lower Conf value indicates lower error and greater confidence in the Fold value. The lack of data for a given variant and Fc ligand indicates either that the fits to the data did not provide a meaningful value, or that the variant was not tested for that particular Fc ligand.

FIG. 25. Data for binding of IgG1 Fc variants to human V158 and F158 FcγRIIIa by AlphaScreen, binding to human V158 FcγRIIIa by SPR, and ADCC in the presence of human effector cells. The values are the fold-affinity (AlphaScreen and SPR) and fold-EC50 (ADCC) relative to WT. Context indicates the antibody variable region in which the data was acquired: a=alemtuzumab, t=trastuzumab, r=rituximab, c=cetuximab, and p=PRO70769.

FIG. 26. Non-naturally occurring modifications provided in FIG. 24, listed according to EU position. Modifications in bolded grey indicate preferred modifications.

FIG. 27. Allotypes and isoallotypes of the human IgG1 constant chain showing the positions and the relevant amino acid substitutions (Gorman & Clark, 1990, Semin Immunol 2(6):457-66). For comparison the amino acids found in the equivalent positions in human IgG2, IgG3 and IgG4 gamma chains are also shown.

FIGS. 28a-28b. Structure of the complex of human IgG1 Fc bound to human FcγRIIIb (pdb accession code 1E4K, Sondermann et al., 2000, Nature 406:267-273), highlighting differences between IgG1 and IgG2 (FIG. 28a), and between IgG1 and IgG4 (FIG. 28b). IgG1 Fc is shown as grey ribbon, FcγRIIIb is shown as black ribbon, and IgG1 residues that differ in amino acid identity from IgG2 (FIG. 28a) and IgG4 (FIG. 28b) are shown as black sticks.

FIGS. 29a and 29b. Competition AlphaScreen™ assay showing binding of IgG1, IgG2, and IgG4 isotypes to V158 FcγRIIIa (FIG. 29a) and protein A (FIG. 29b). The variable region of the antibodies is that of the anti-Her2 antibody trastuzumab. In the presence of competitor antibody, a characteristic inhibition curve is observed as a decrease in luminescence signal. These data were normalized to the maximum and minimum luminescence signal provided by the baselines at low and high concentrations of competitor antibody respectively. The curves represent the fits of the data to a one site competition model using nonlinear regression.

FIGS. 30a-30b. Competition AlphaScreen assay showing binding of WT and variant IgG1, IgG2, and IgG4 antibodies to human V158 FcγRIIIa (FIG. 30a) and human FcγRI (FIG. 30b). The variable region of the antibodies is that of the anti-Her2 antibody trastuzumab.

FIG. 31. SPR (Surface Plasmon Resonance) data showing binding of WT and variant IgG1, IgG2, and IgG4 antibodies to human V158 FcγRIIIa. The variable region of the antibodies is that of the anti-Her2 antibody trastuzumab.

FIGS. 32a-32b. IgG1 variants with isotypic and/or novel amino acid modifications. The amino acid sequences of the human immunoglobulin isotypes IgG1, IgG2, IgG3, and IgG4 are aligned according to FIG. 1. FIG. 32a provides the sequences of the CH1 domain and hinge regions, and FIG. 32b provides the sequences of the CH2 and CH3 domains. The sequence of IgG1 is provided explicitly, and residues in the rows labeled “IgG2”, “IgG3”, and “IgG4” provide the amino acid identity at EU positions where they differ from IgG1; these modifications are isotypic modifications. Residues listed in the rows labeled “Novel” indicate novel modifications for human IgG1; these novel modifications are those indicated as preferred in FIG. 26.

FIGS. 33a-33b. IgG2 variants with isotypic and/or non-naturally occurring modifications. The amino acid sequences of the human immunoglobulin isotypes IgG2, IgG1, IgG3, and IgG4 are aligned according to FIG. 1. FIG. 33a provides the sequences of the CH1 domain and hinge regions, and FIG. 33b provides the sequences of the CH2 and CH3 domains. The sequence of IgG2 is provided explicitly, and residues in the rows labeled “IgG1”, “IgG3”, and “IgG4” provide the amino acid identity at EU positions where they differ from IgG2; these modifications are isotypic modifications. Residues listed in the rows labeled “Novel” indicate novel modifications for human IgG2; these novel modifications are those indicated as preferred in FIG. 26.

FIGS. 34a-34b. IgG3 variants with isotypic and/or non-naturally occurring modifications. The amino acid sequences of the human immunoglobulin isotypes IgG3, IgG1, IgG2, and IgG4 are aligned according to FIG. 1. FIG. 34a provides the sequences of the CH1 domain and hinge regions, and FIG. 34b provides the sequences of the CH2 and CH3 domains. The sequence of IgG3 is provided explicitly, and residues in the rows labeled “IgG1”, “IgG2”, and “IgG4” provide the amino acid identity at EU positions where they differ from IgG3; these modifications are isotypic modifications. Residues listed in the rows labeled “Novel” indicate novel modifications for human IgG3; these novel modifications are those indicated as preferred in FIG. 26.

FIGS. 35a-35b. IgG4 variants with isotypic and/or non-naturally occurring modifications. The amino acid sequences of the human immunoglobulin isotypes IgG4, IgG1, IgG2, and IgG3 are aligned according to FIG. 1. FIG. 35a provides the sequences of the CH1 domain and hinge regions, and FIG. 35b provides the sequences of the CH2 and CH3 domains. The sequence of IgG4 is provided explicitly, and residues in the rows labeled “IgG1”, “IgG2”, and “IgG3” provide the amino acid identity at EU positions where they differ from IgG4; these modifications are isotypic modifications. Residues listed in the rows labeled “Novel” indicate novel modifications for human IgG4; these novel modifications are those indicated as preferred in FIG. 26.

FIG. 36. Anti-Her2 IgG2 Variants. Novel modifications and isotypic modifications are provided for each variant, all constructed in the context of the human IgG2 isotype. The variable region (VHVL), CH1 domain (CH1), hinge region (hinge), and Fc region (Fc) are described for each variant, and the full constant region is labeled (WT IgG2, IgG2 ELLGG, or IgG(1/2) ELLGG) accordingly.

FIG. 37. Competition AlphaScreen assay showing binding of WT and IgG variant antibodies to human V158 FcγRIIIa. The variable region of the antibodies is that of the anti-Her2 antibody trastuzumab.

FIG. 38. Anti-CD30 IgG(1/2) ELLGG Variants. Novel modifications and isotypic modifications are provided for each variant. All IgG variants comprise the variable region of the anti-CD30 antibody H3.69_V2_L3.71 AC10. The variants comprise the IgG(1/2) ELLGG constant region as described in FIG. 37, and potentially one or more additional isotypic modifications and/or one or more novel modifications.

FIGS. 39a-39c. Competition AlphaScreen assay showing binding of WT and variant IgG antibodies to human V158 FcγRIIIa. IgG variants comprise the constant region of either IgG1 or IgG(1/2) ELLGG plus the indicated modifications. With the exception of I332E and S239D/I332E IgG1, all IgG variants comprise the variable region of the anti-CD30 antibody H3.69_V2_L3.71 AC10. Variants I332E IgG1 and S239D/I332E IgG1 comprise the variable region of the anti-CD30 antibody H3.69 L3.71 AC10.

FIG. 40. Data for binding of anti-CD30 IgG variants to human V158 FcγRIIIa as measured by the competition AlphaScreen. For each variant are provided the IC50 (M) and Fold IC50 relative to H3.69_V2_L3.71 AC10 IgG1.

FIGS. 41a-41d. Cell-based ADCC assay of WT and variant IgGs with the variable region of the anti-CD30 antibody H3.69_V2_L3.71 AC10 or H3.69 L3.71 AC10 (I33E and S239D/I332E IgG1). ADCC was measured by LDH activity using the Cytotoxicity Detection Kit (LDH, Roche Diagnostic Corporation, Indianapolis, Ind.) or the DELFIA® EuTDA-based cytotoxicity assay (Perkin Elmer, MA). For all assays, target cells were L540 Hodgkin's lymphoma cells and effector cells were human PBMCs. The figures show the dose-dependence of ADCC on antibody concentration for the indicated antibodies, normalized to the minimum and maximum fluorescence signal for each particular curve, provided by the baselines at low and high antibody concentrations respectively. The curves represent the fits of the data to a sigmoidal dose-response model using nonlinear regression.

FIG. 42. Anti-CD20 IgG(1/2) ELLGG Variants. Novel modifications and isotypic modifications are provided for each variant. All IgG variants comprise the variable region of the anti-CD20 antibody rituximab. The IgG variants comprise the IgG(1/2) ELLGG constant region and potentially one or more novel modifications.

FIG. 43. Cell-based ADCC assay of WT and variant IgGs with the variable region of the anti-CD20 antibody rituximab. ADCC was measured by LDH activity using the Cytotoxicity Detection Kit (LDH, Roche Diagnostic Corporation, Indianapolis, Ind.) according to the manufacturer's instructions, with WIL2-S lymphoma target cells and human PBMCs as effector cells.

FIG. 44. Anti-CD20 IgG(1/2) ELLGG Variants. Novel modifications and isotypic modifications are provided for each variant. All IgG variants comprise the variable region of the anti-CD20 antibody PRO70769. The variants comprise the IgG(1/2) ELLGG constant region and potentially one or more additional isotypic modifications and/or one or more novel modifications.

FIG. 45. Competition AlphaScreen assay showing binding of anti-CD20 IgG variant antibodies to human V158 FcγRIIIa. IgG variants comprise the constant region of either IgG1 or IgG(1/2) ELLGG plus the indicated modifications. All IgG variants comprise the variable region of the anti-CD20 antibody PRO70769.

FIG. 46. Cell-based ADCC assay of WT and variant IgGs with the variable region of the anti-CD20 antibody PRO70769. ADCC was measured using the DELFIA® EuTDA-based cytotoxicity assay with WIL2-S lymphoma target cells and human PBMCs as effector cells.

FIG. 47. Cell-based CDC assay of WT and variant IgGs with the variable region of the anti-CD20 antibody PRO70769. CDC assays were performed using Alamar Blue to monitor lysis of antibody—opsonized WIL2-S lymphoma cells by human serum complement (Quidel, San Diego, Calif.). The dose-dependence on antibody concentration of complement-mediated lysis is shown, normalized to the minimum and maximum fluorescence signal for each particular curve, provided by the baselines at low and high antibody concentrations respectively. The curves represent the fits of the data to a sigmoidal dose-response model using nonlinear regression.

FIGS. 48a-48h. Amino acid sequences of variable light (VL) and heavy (VH) chains used in the present invention. (SEQ ID NOS:12-19)

FIGS. 49a-49g. Amino acid sequences of constant light and heavy chains used in the present invention. (SEQ ID NOS: 7-11 and 20-21) EU residues 233-236 are bolded in the IgG(1/2) (FIG. 490 (SEQ ID NO: 20) and IgG(1/2) ELLGG (FIG. 49g) (SEQ ID NO: 21) sequences.

FIGS. 50a-50d. Amino acid sequences of IgG variant antibodies of the present invention. (SEQ ID NOS: 22-25) FIGS. 50a and 50b (SEQ ID NOS: 22 AND 23) provide the light and heavy chains respectively of an anti-CD20 antibody including the constant region IgG(1/2) ELLGG S239D/I332E/G327A. FIGS. 50c and 350d (SEQ ID NOS: 24 AND 25) provide the light and heavy chains respectively of an anti-CD30 antibody including the constant region IgG(1/2) ELLGG S239D/I332E/G327A. EU residues 233-236, 239, 327, and 332 are bolded in the heavy chain sequences in FIGS. 50b and 50d. (SEQ ID NOS: 23 and 25)

FIG. 51. FcγR-dependent effector functions and potentially relevant FcγRs for select immune cell types that may be involved in antibody-targeted tumor therapy. The third column presents interactions that may regulate activation or inhibition of the indicated cell type, with those that are thought to be particularly important highlighted in bold.

FIG. 52. Sequence alignment of human FcγRs. Differences from FcγRIIb are highlighted in gray, and positions at the Fc interface are indicated with an i. Numbering is shown according to both the 1IIS.pdb and 1E4K.pdb structures.

FIG. 53. Structure of the Fc/FcγR interface indicating differences between the FcγRIIa and FcγRIIb structures, and proximal Fc residues. The structure is that of the 1E4K.pdb Fc/FcγRIIIb complex. FcγR is represented by black ribbon and Fc is represented as gray ribbon. FcγR positions that differ between FcγRIIa and FcγRIIb are shown in gray, and proximal Fc residues to these FcγR residues are shown in black.

FIG. 54a-54b. Binding of select anti-CD20 Fc variants to human R131 FcγRIIa (FIG. 54a) and FcγRIIb (FIG. 54b) as measured by competition AlphaScreen™ assay. In the presence of competitor antibody (Fc variant or WT) a characteristic inhibition curve is observed as a decrease in luminescence signal. The binding data were normalized to the maximum and minimum luminescence signal for each particular curve, provided by the baselines at low and high antibody concentrations respectively. The curves represent the fits of the data to a one site competition model using nonlinear regression.

FIG. 55. Summary of FcγR binding properties of anti-CD20 Fc variants for binding to human FcγRI, R131 FcγRIIa, H131 FcγRIIa, FcγRIIb, and V158 FcγRIIIa. Shown are the IC50s obtained from the AlphaScreen, and the Fold(IC50) relative to WT. Duplicate binding results, shown on separate lines, are provided for some variants.

FIG. 56. Binding of select anti-EGFR Fc variants to human FcγRI, R131 and H131 FcγRIIa, FcγRIIb, and V158 FcγRIIIa as measured by competition AlphaScreen assay.

FIG. 57. Summary of FcγR binding properties of anti-EGFR Fc variants for binding to human FcγRI, R131 FcγRIIa, H131 FcγRIIa, FcγRIIb, and V158 FcγRIIIa. Shown are the IC50s obtained from the AlphaScreen, and the Fold(IC50) relative to WT.

FIG. 58. Surface Plasmon Resonance (SPR) (BIAcore) sensorgrams of binding of select anti-EpCAM Fc variants to human R131 FcγRIIa.

FIG. 59. Affinity data for binding of anti-EpCAM Fc variants to human FcγRI, R131 and H131 FcγRIIa, FcγRIIb, V158 FcγRIIIa, and F158 FcγRIIIa as determined by SPR. Provided are the association (ka) and dissociation (kd) rate constants, the equilibrium dissociation constant (K_{D}), the Fold K_{D} relative to WT, and the negative log of the K_{D}

(−log(K_{D})).

FIG. 60. Plot of the negative log of the K_{D} for binding of select anti-EpCAM Fc variants to human FcγRI, R131 FcγRIIa, H131 FcγRIIa, FcγRIIb, and V158 FcγRIIIa.

FIG. 61a-61c. Affinity differences between activating and inhibitory FcγRs for select anti-EpCAM Fc variants. FIG. 61a shows the absolute affinity differences between the activating receptors and the inhibitory receptor FcγRIIb. The top graph shows the affinity differences between both isoforms of FcγRIIa and FcγRIIb, represented mathematically as

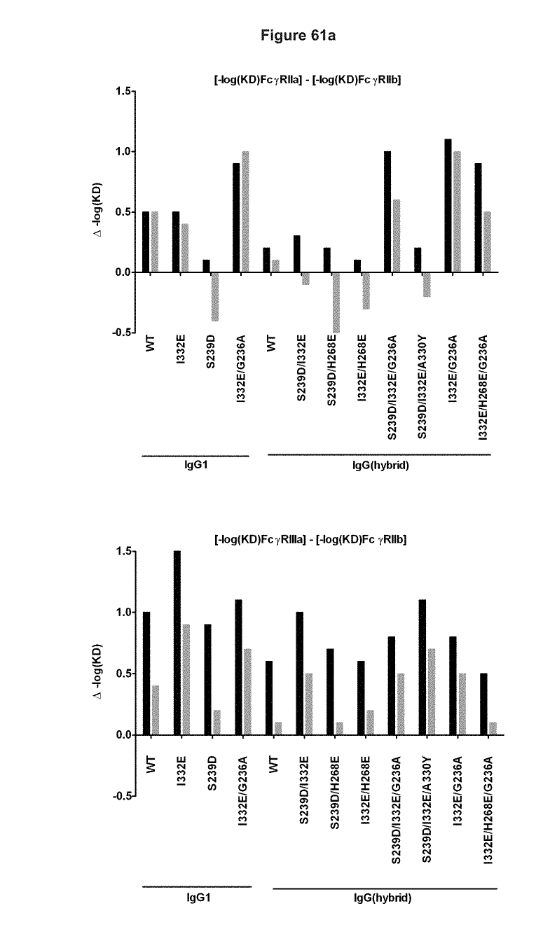

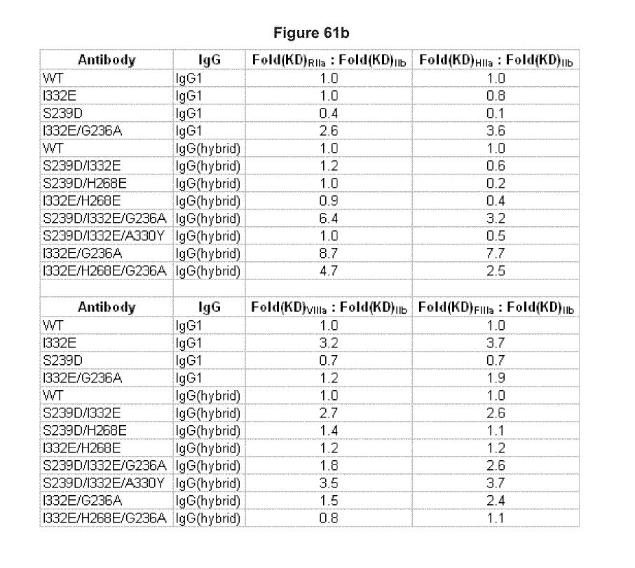

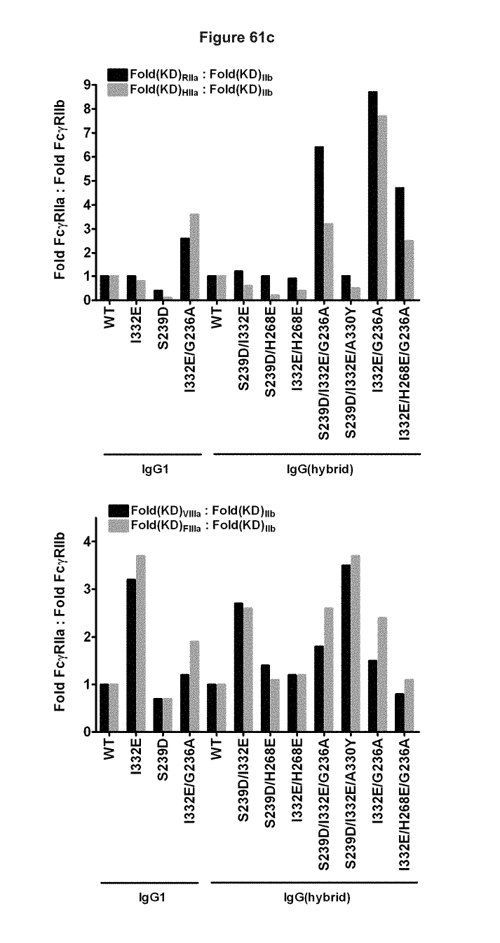

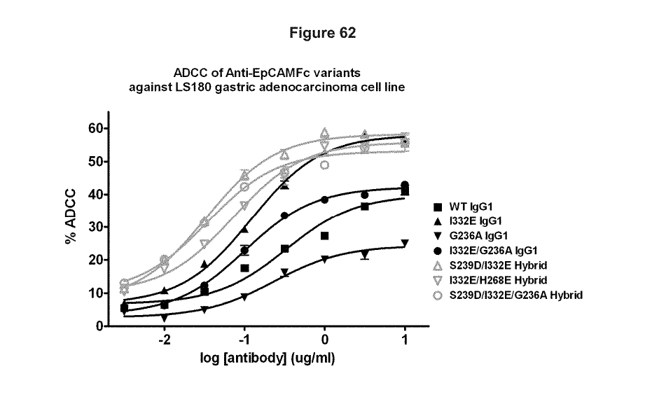

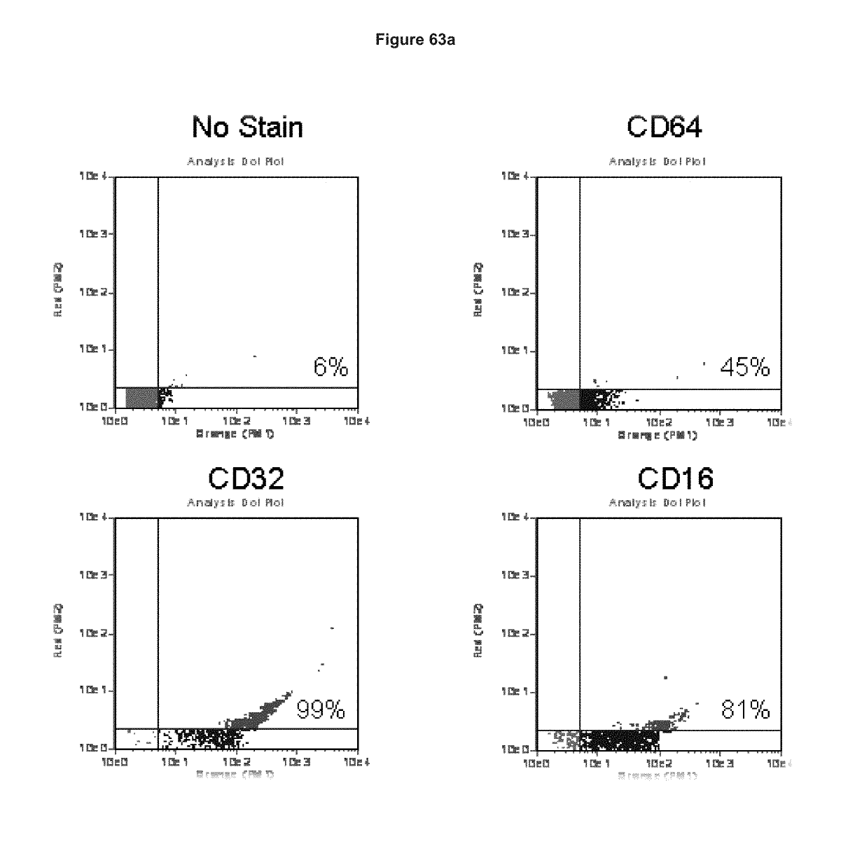

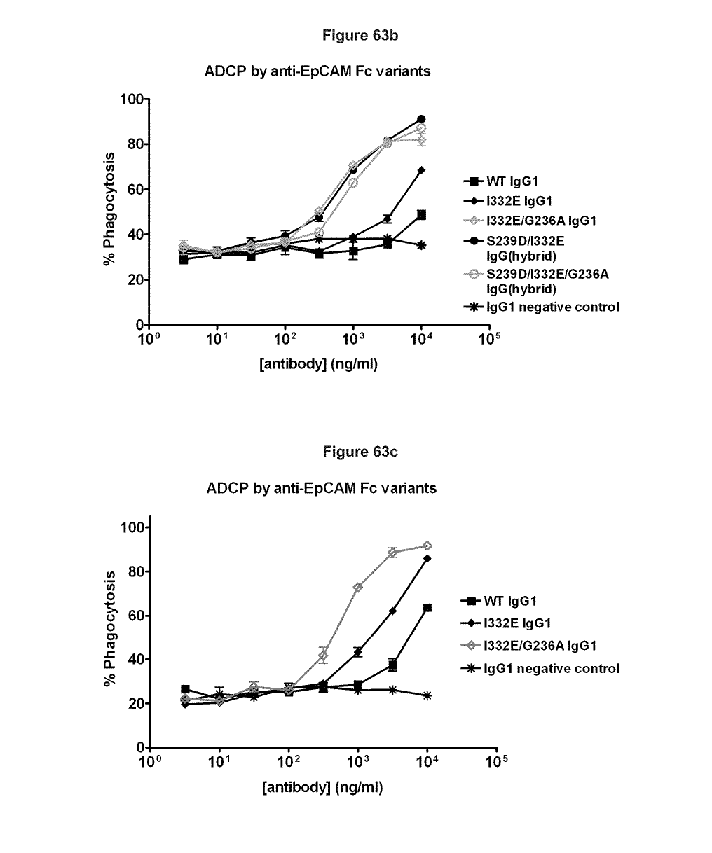

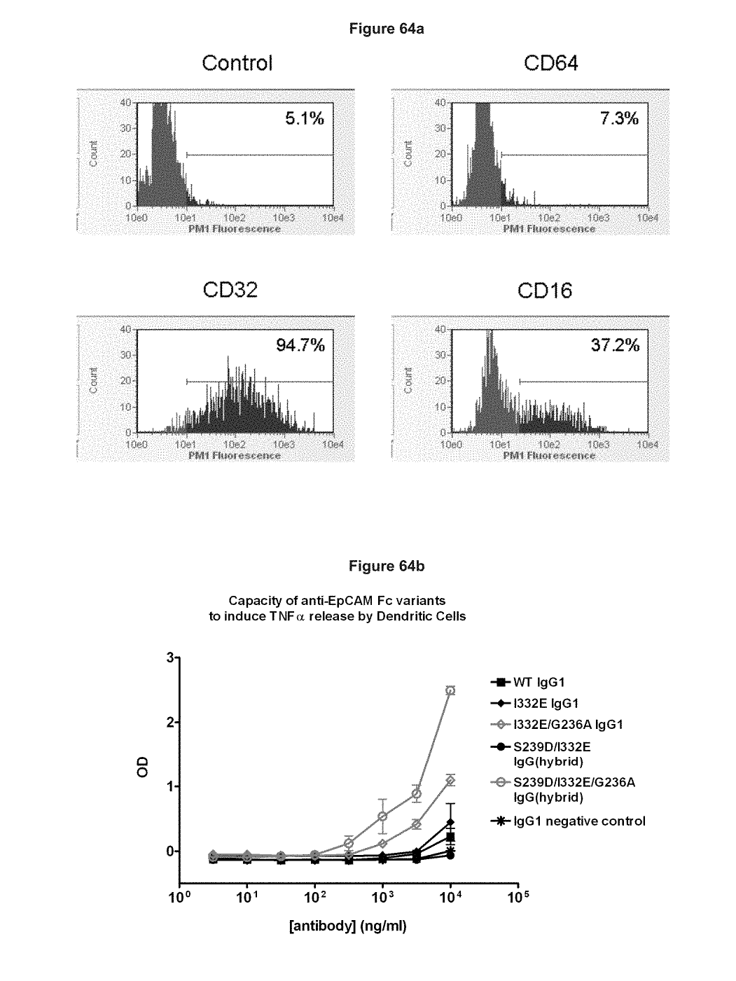

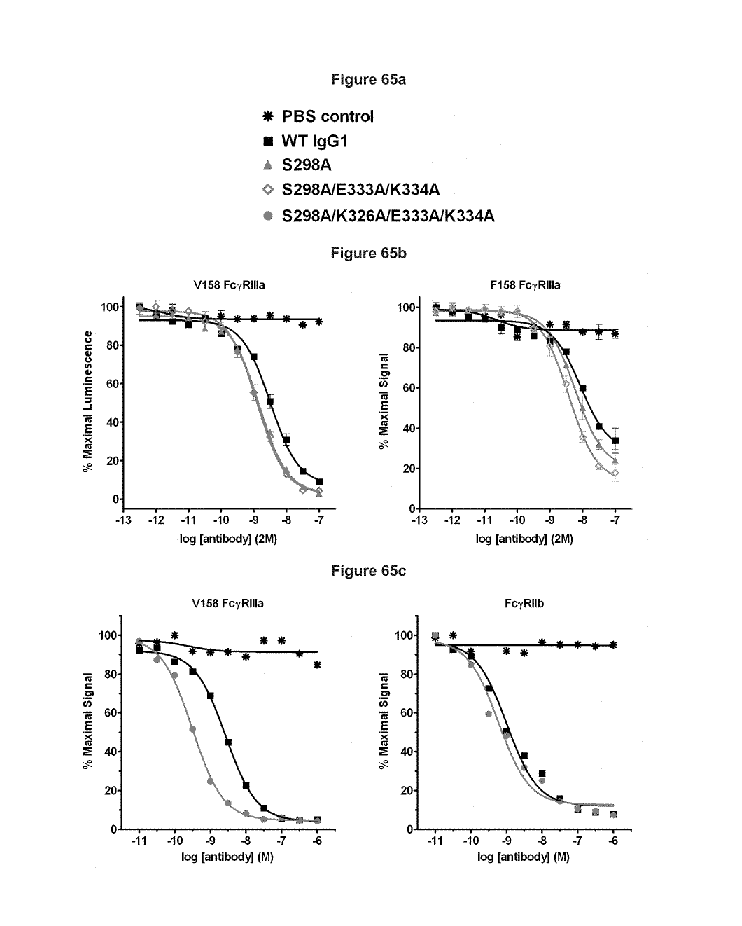

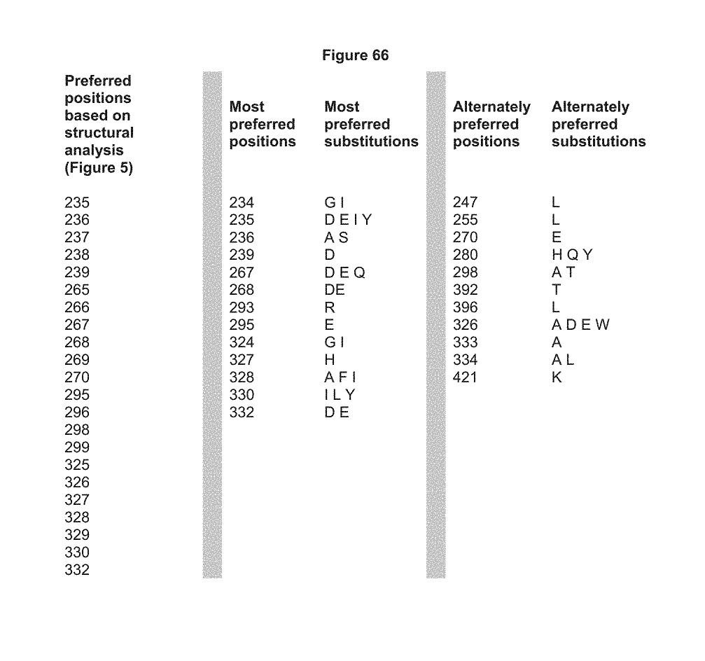

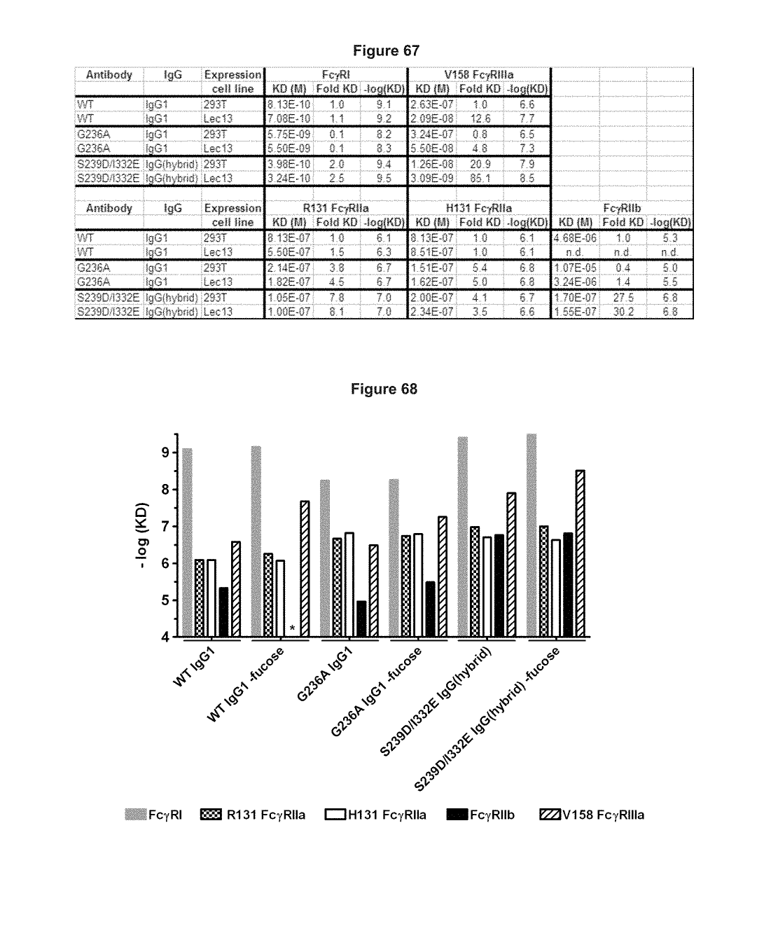

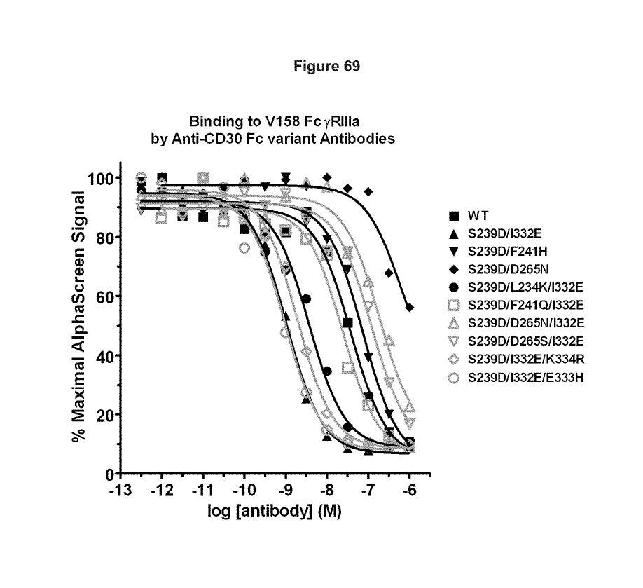

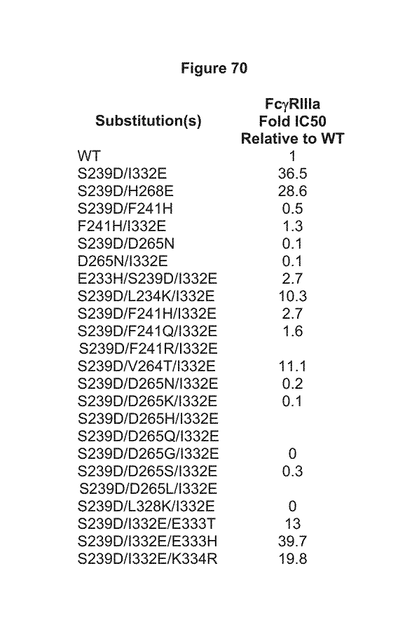

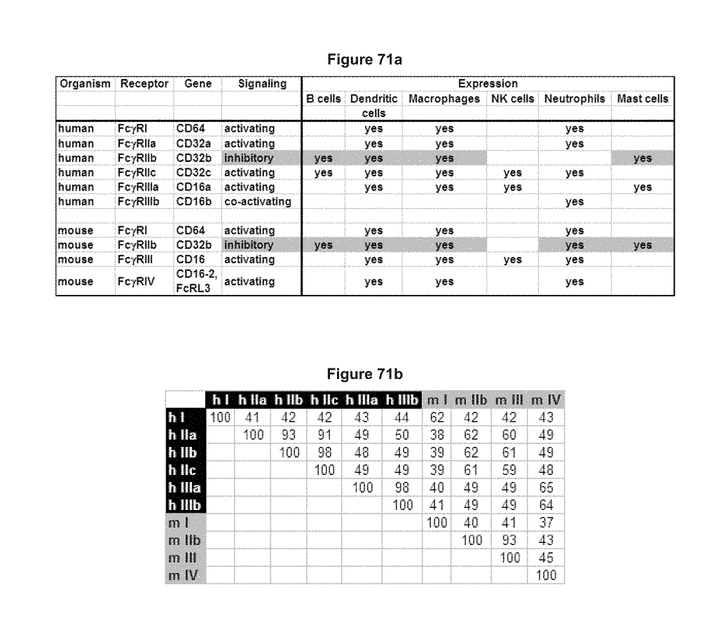

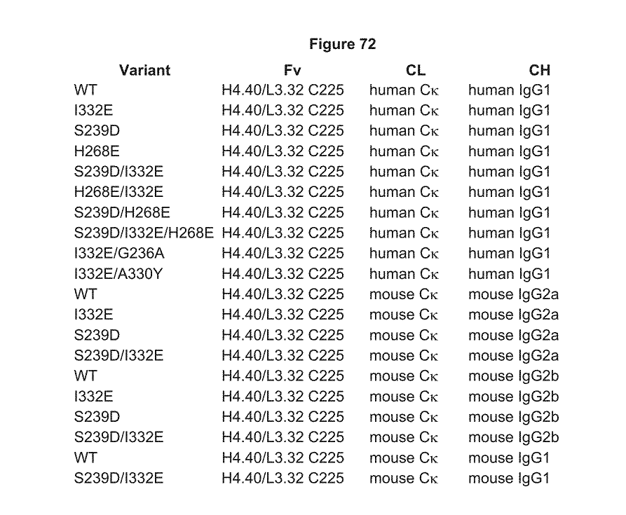

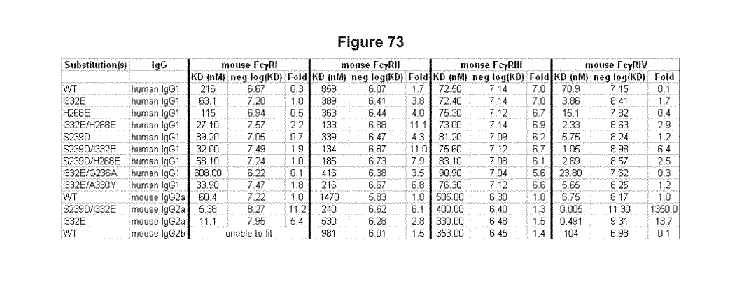

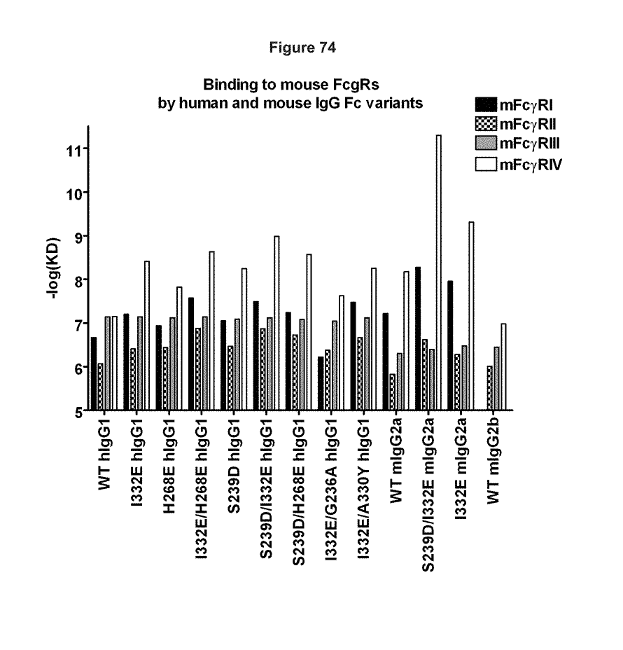

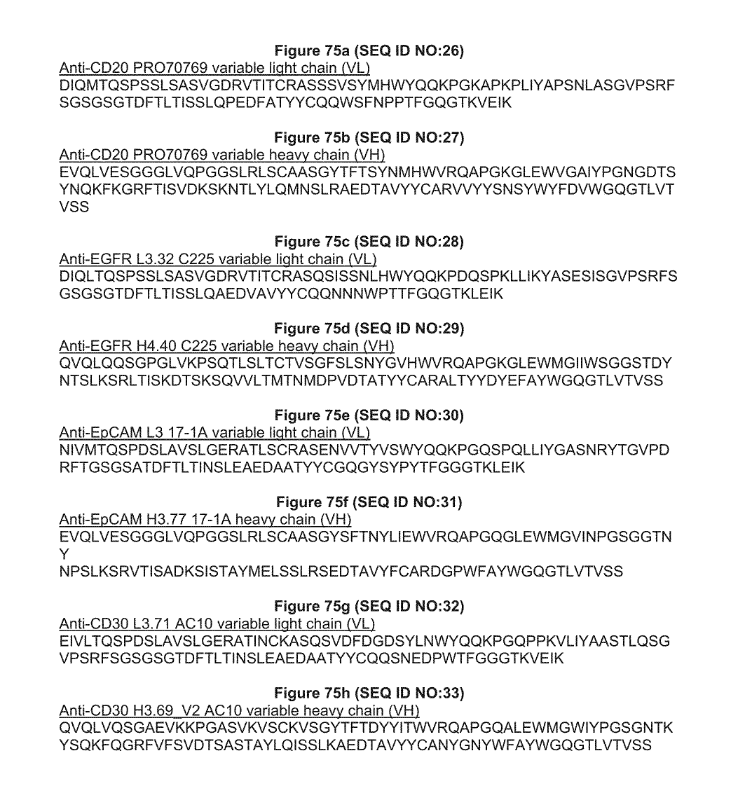

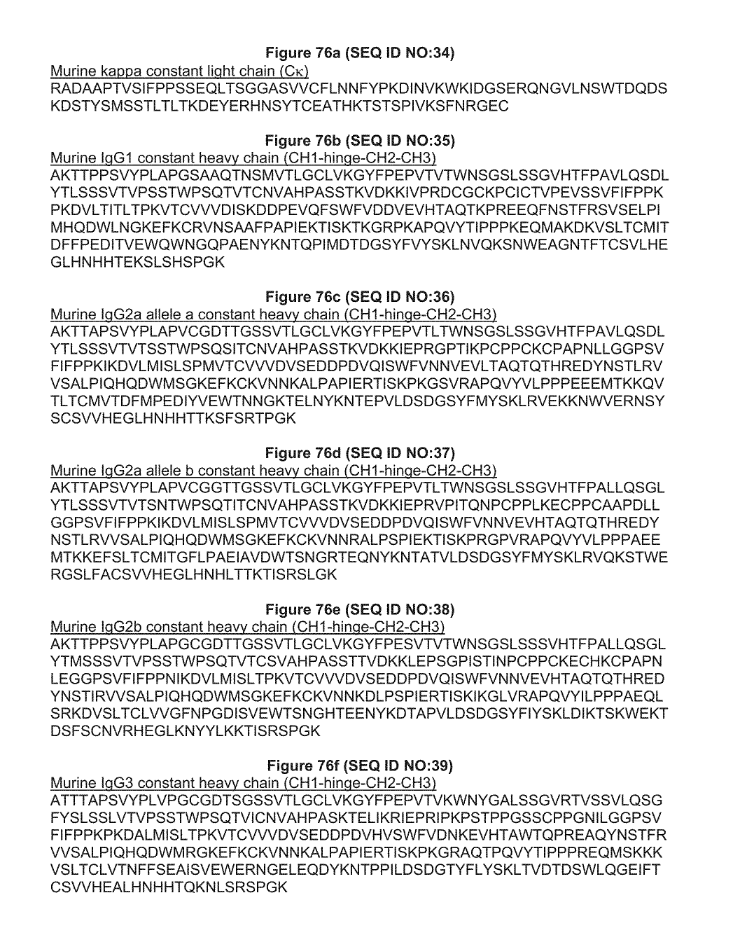

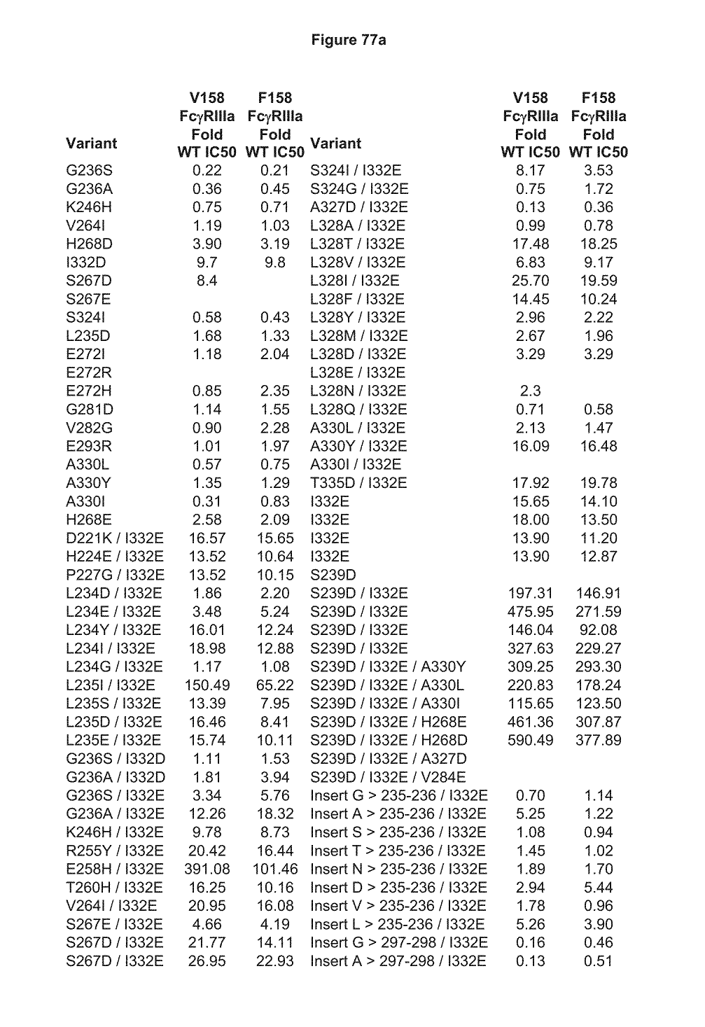

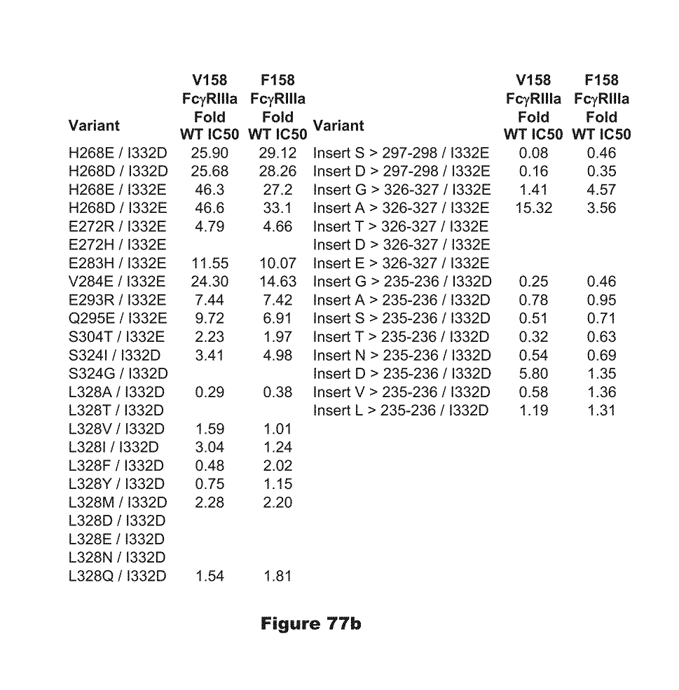

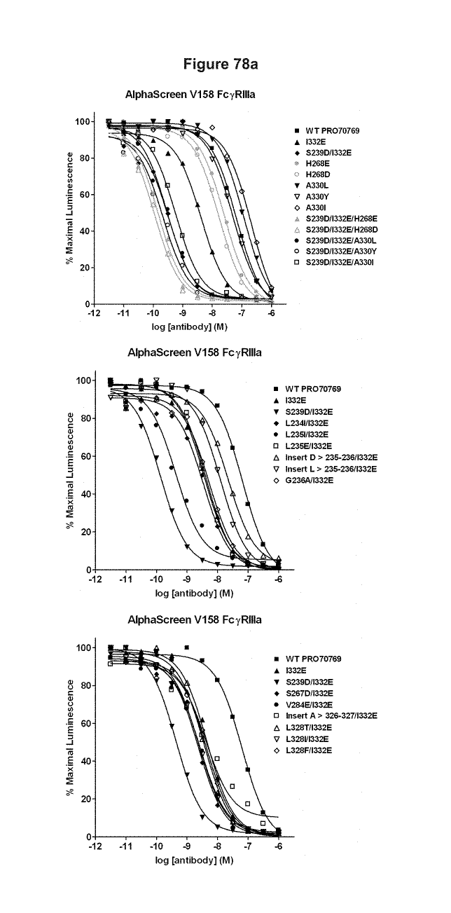

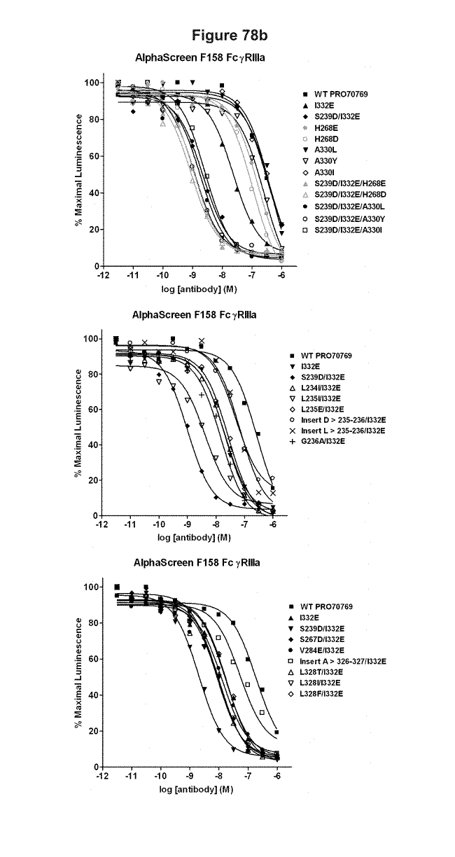

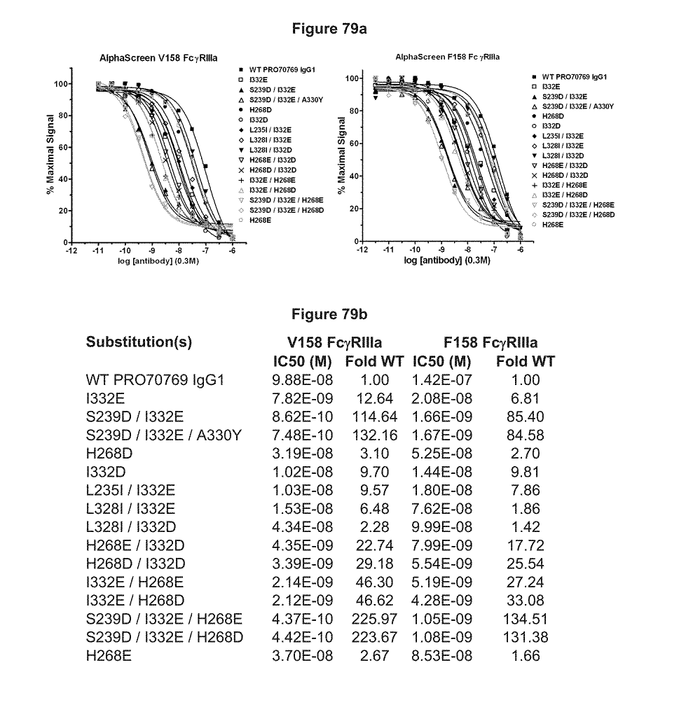

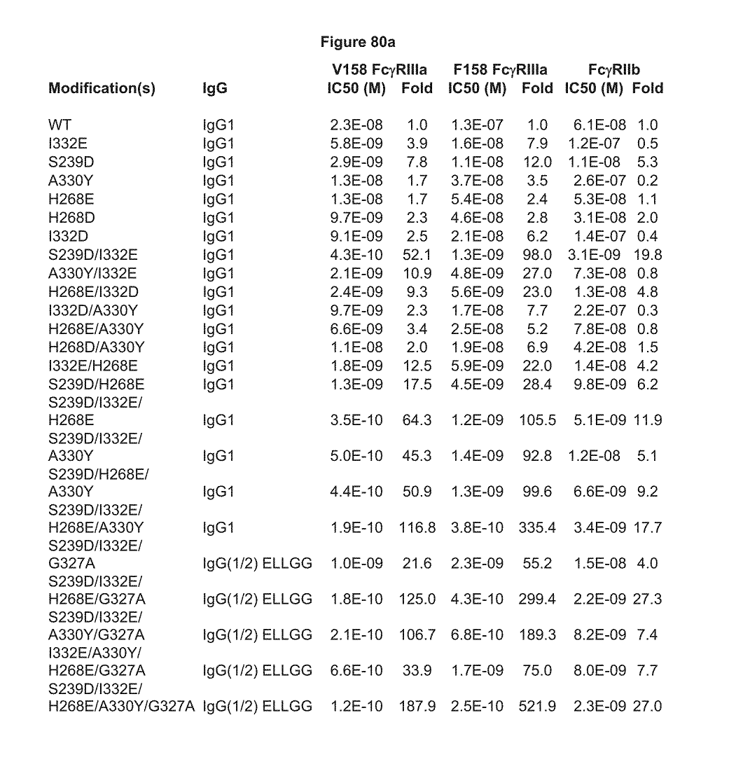

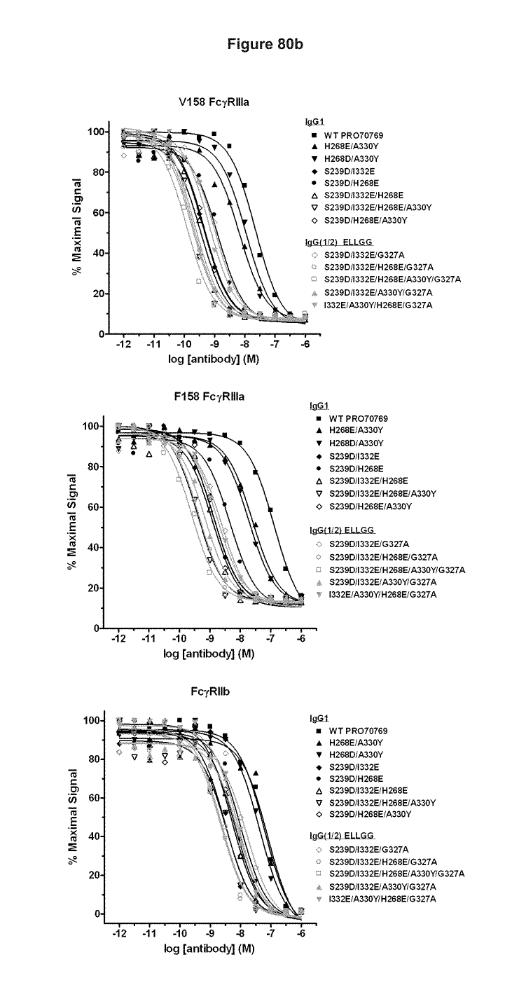

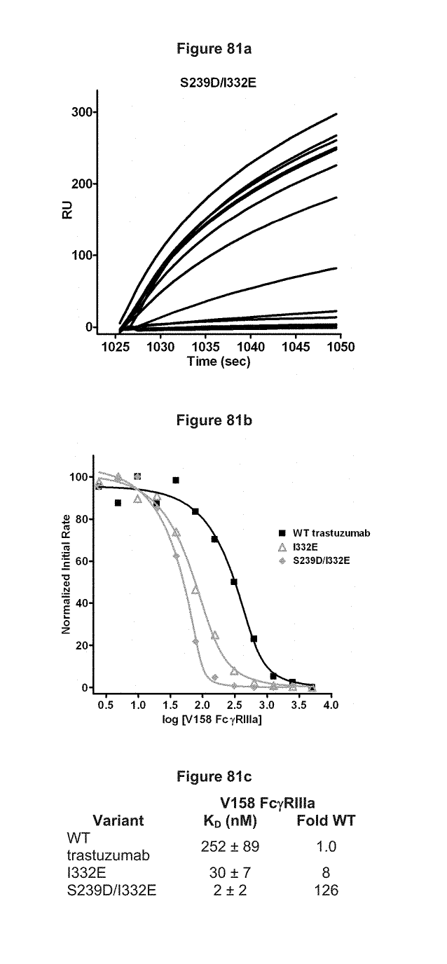

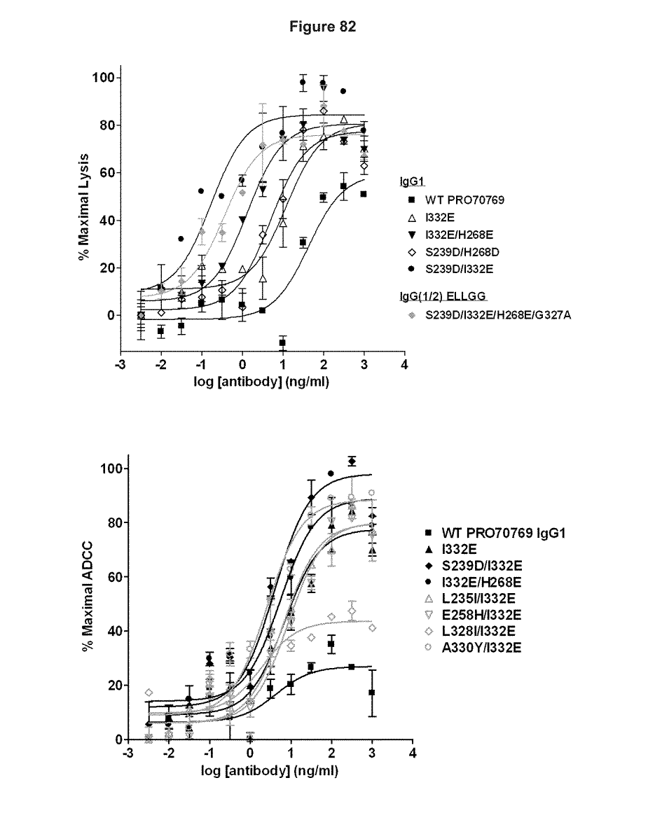

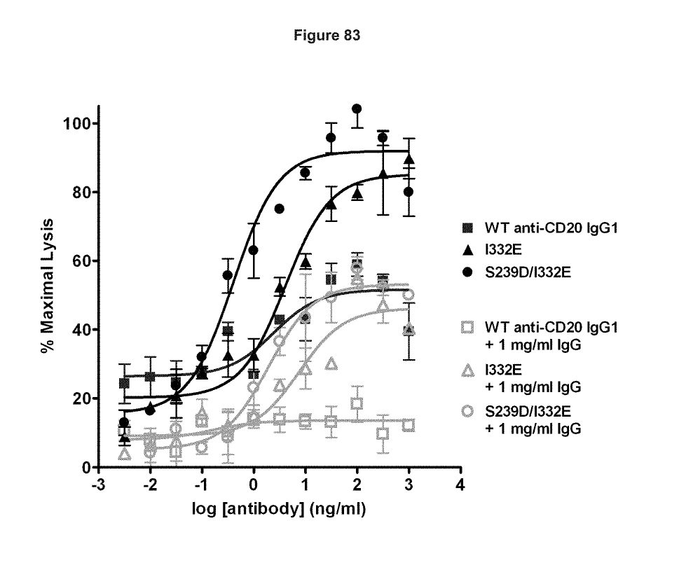

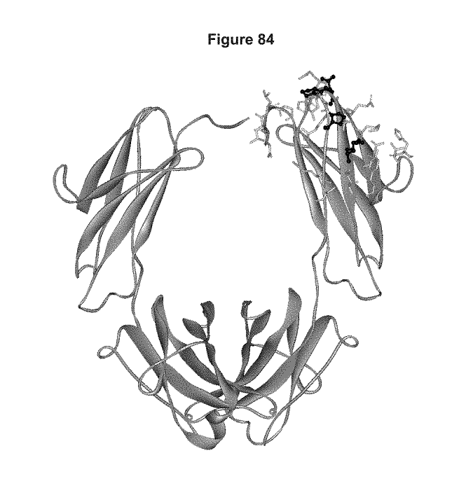

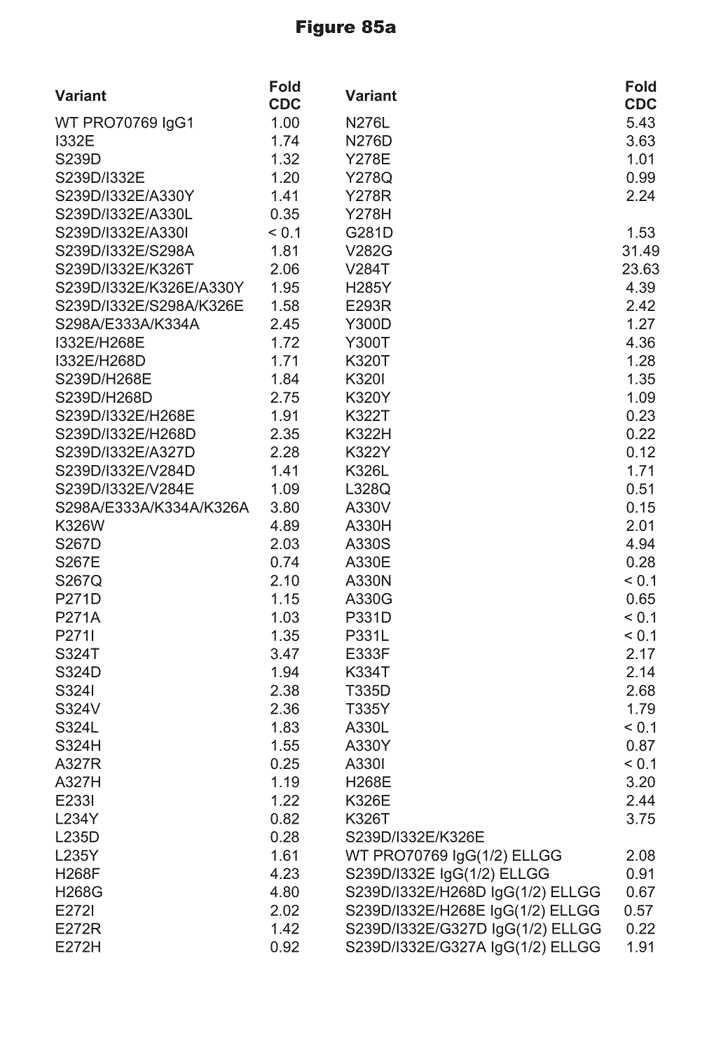

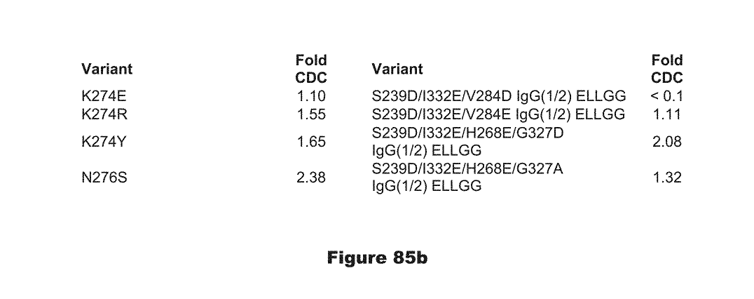

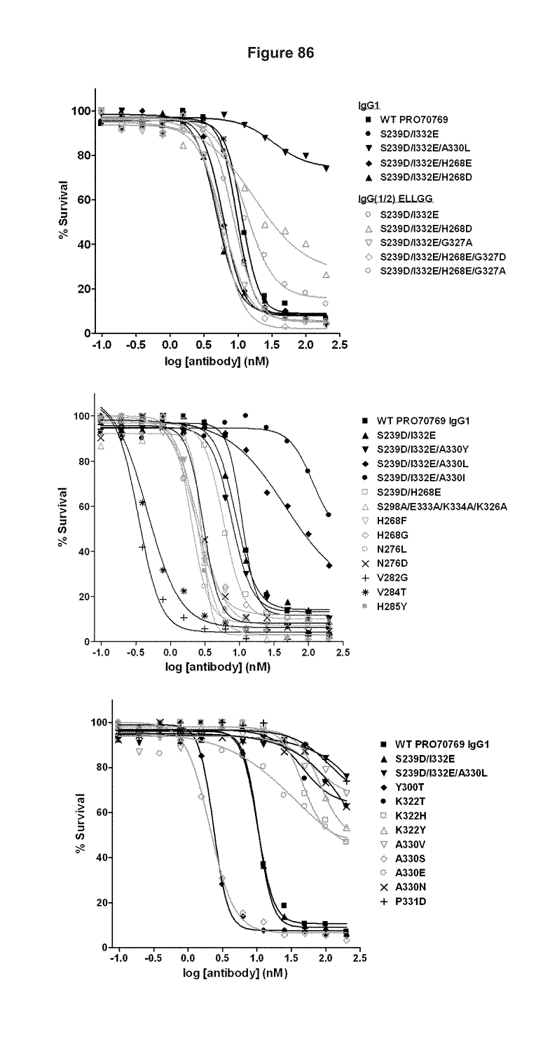

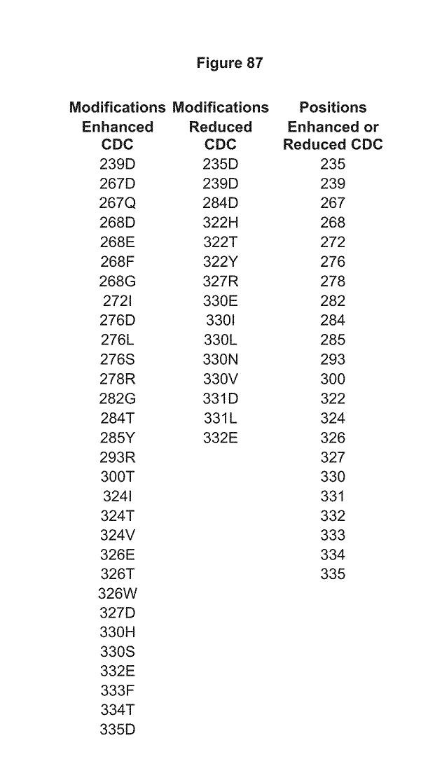

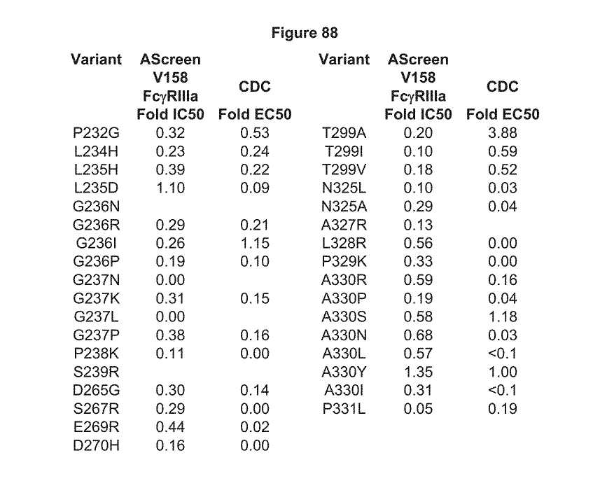

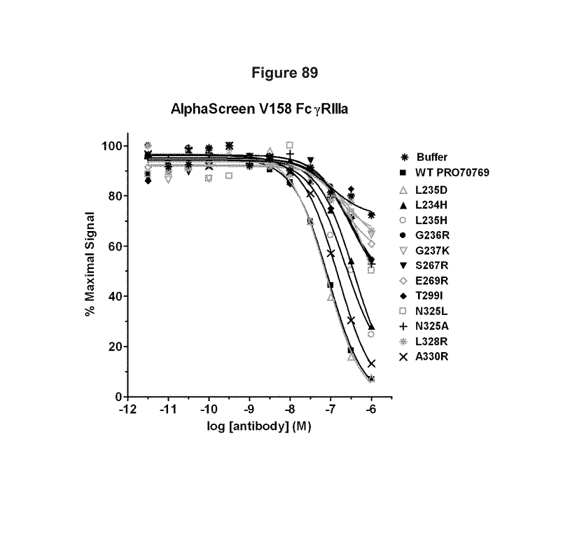

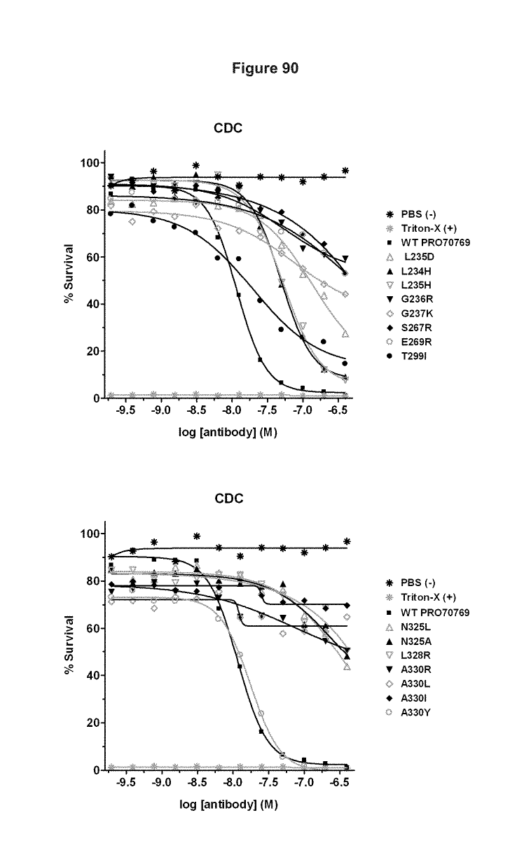

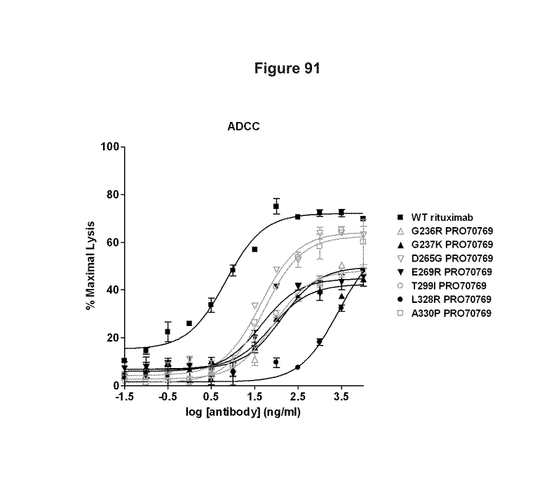

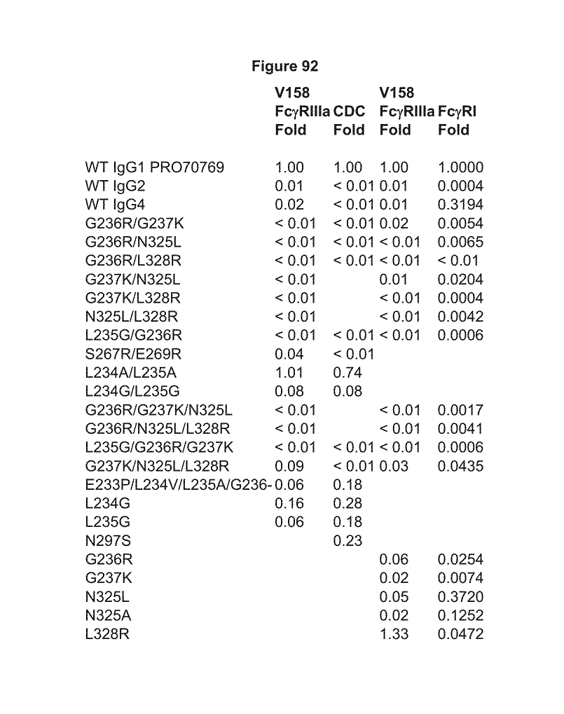

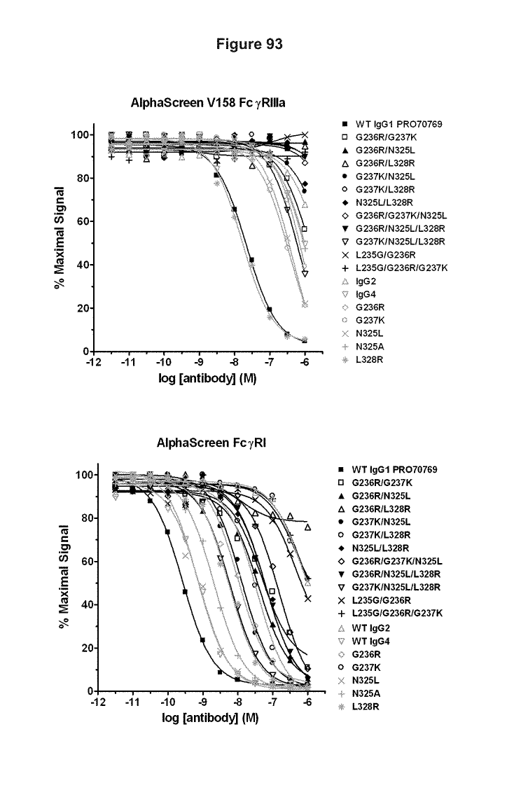

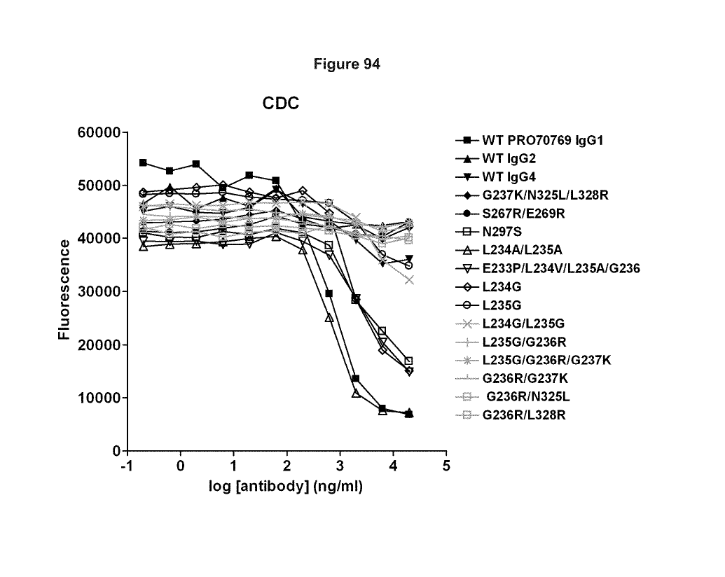

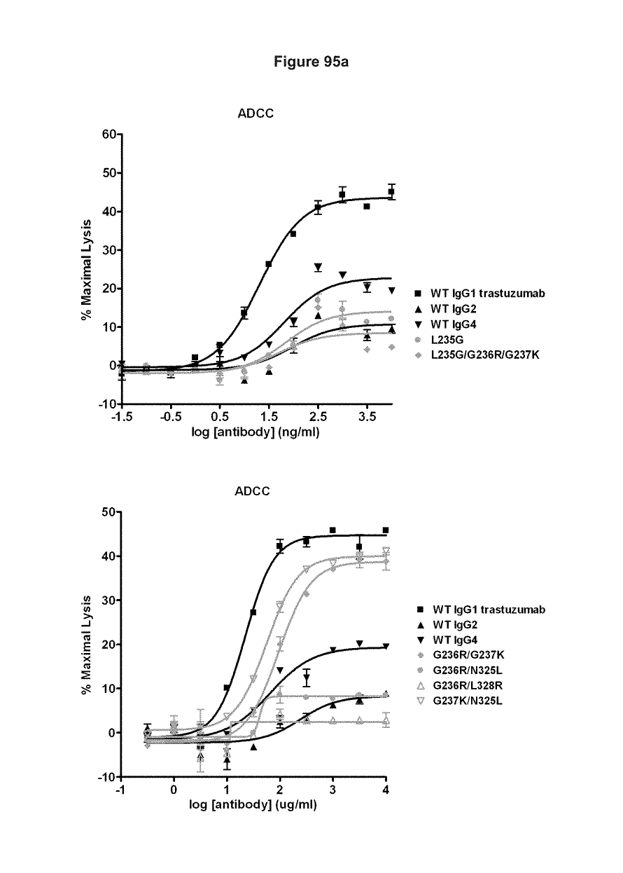

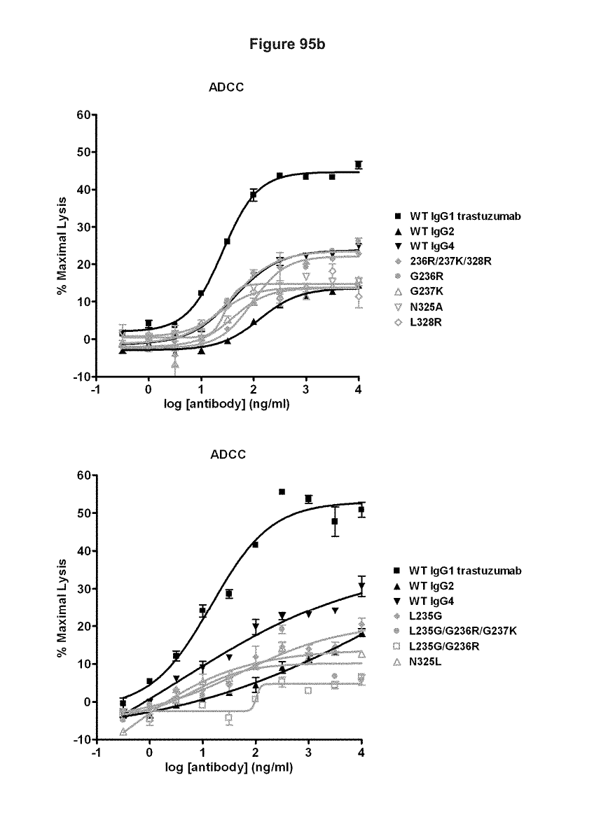

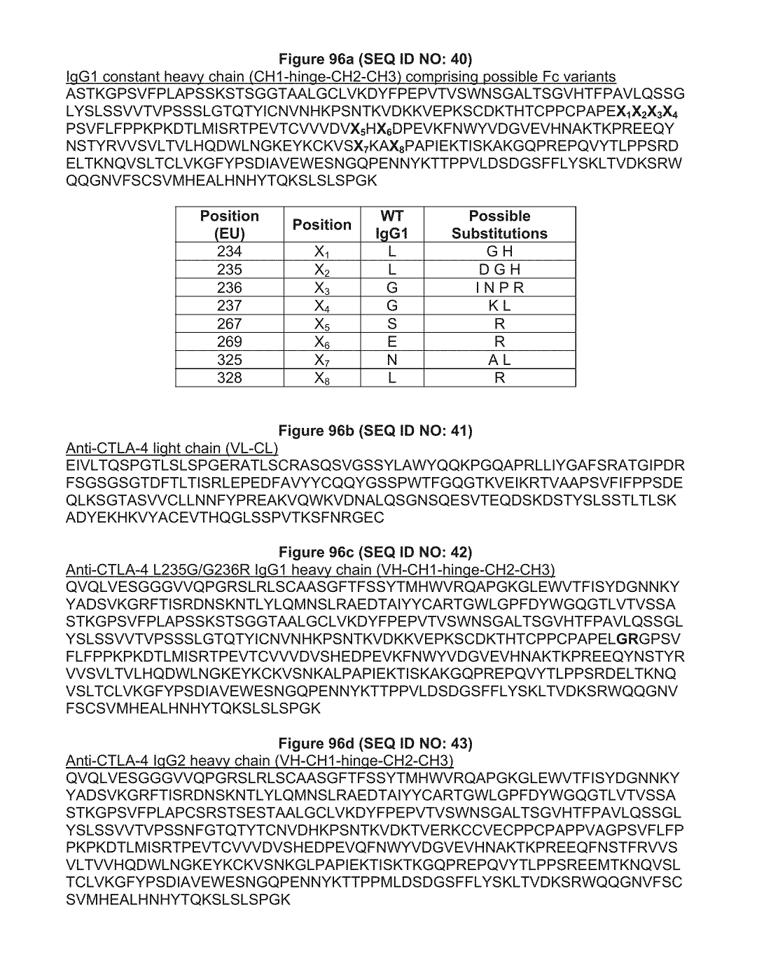

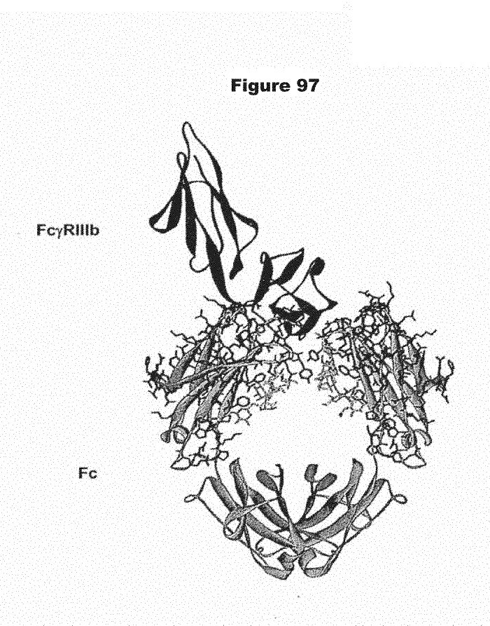

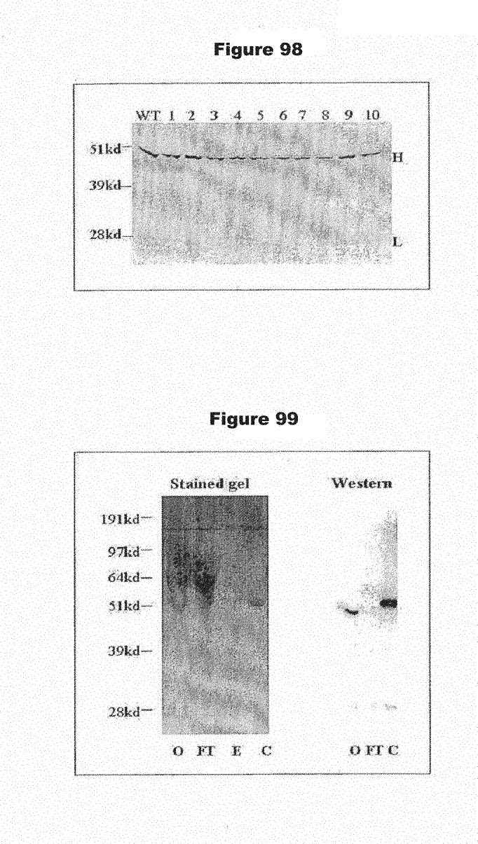

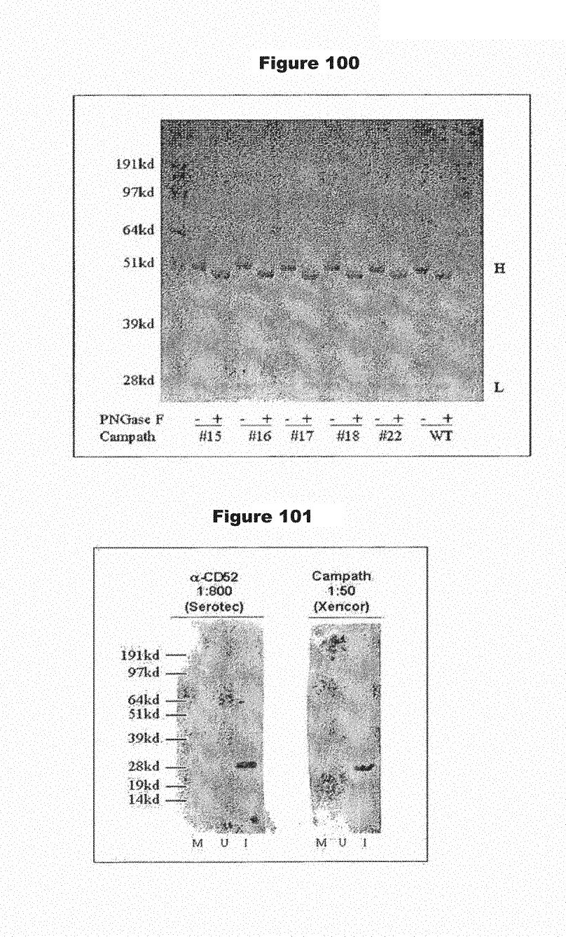

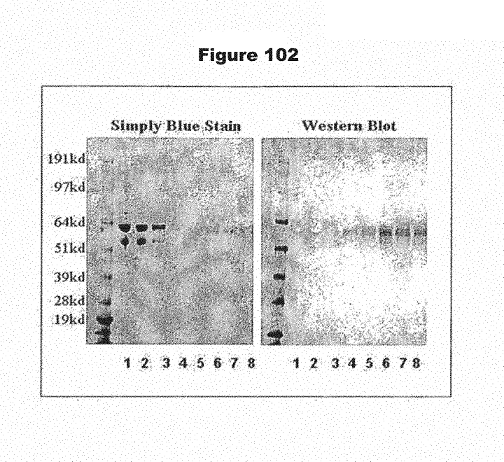

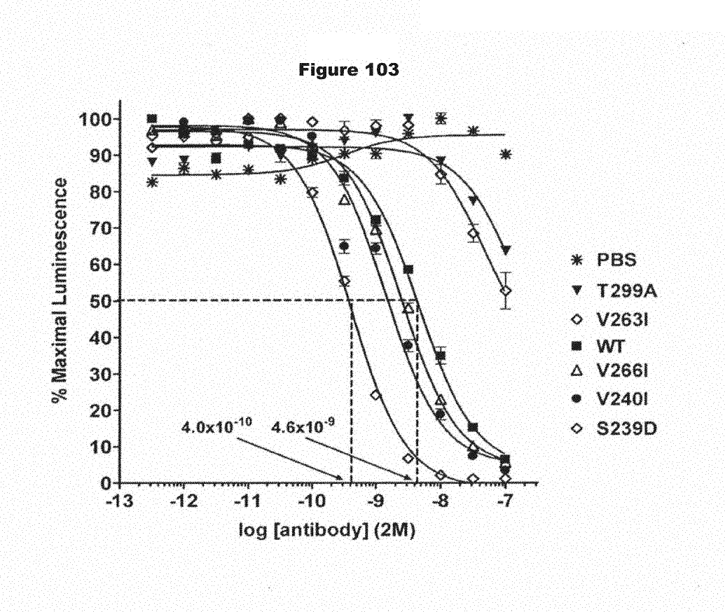

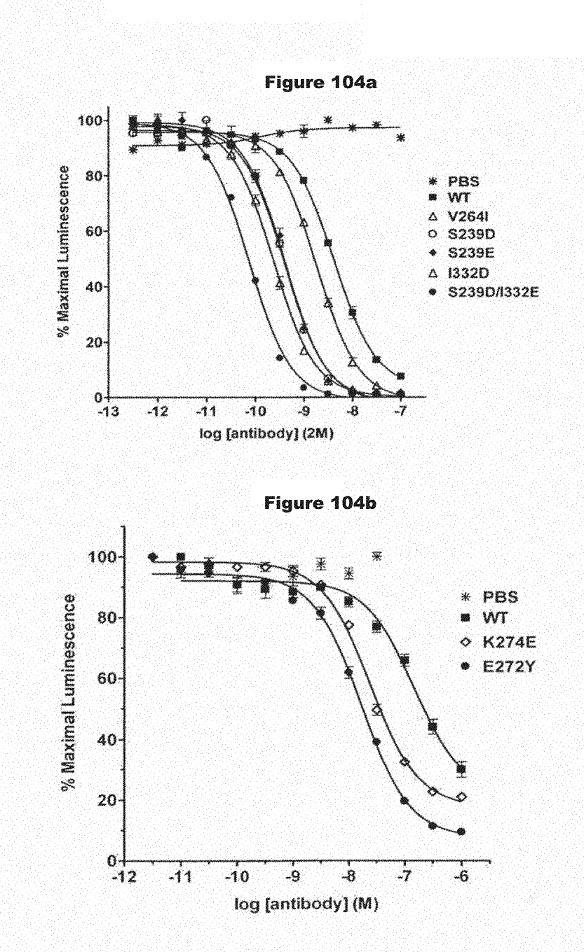

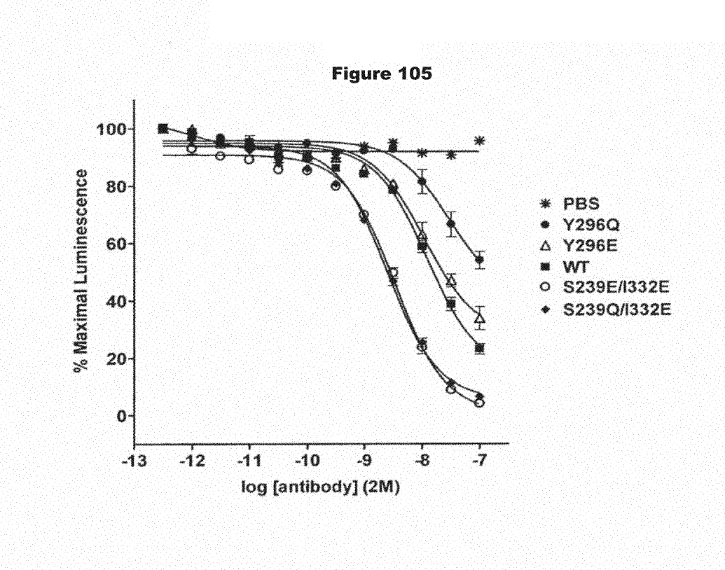

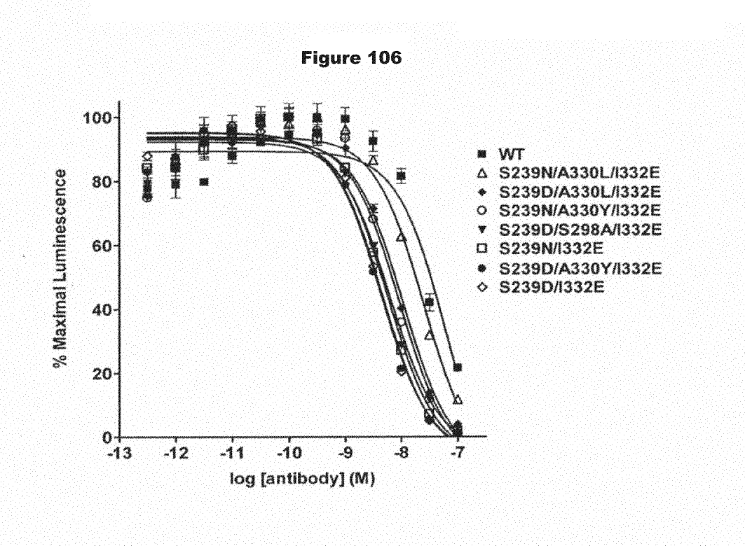

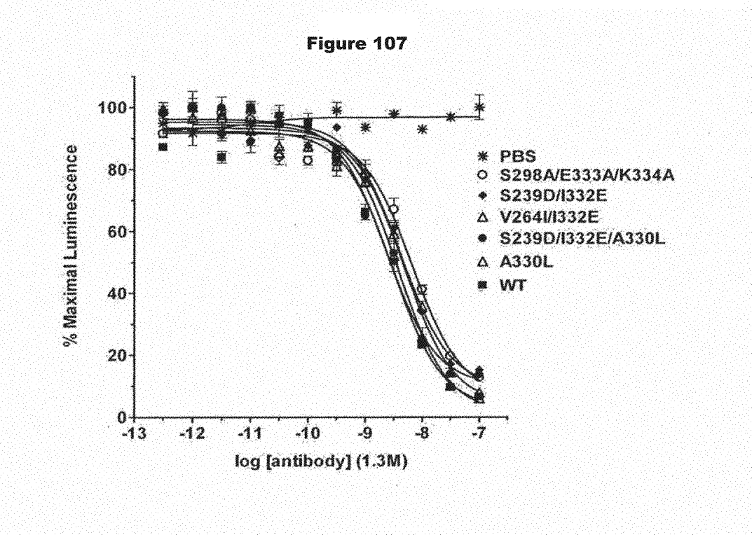

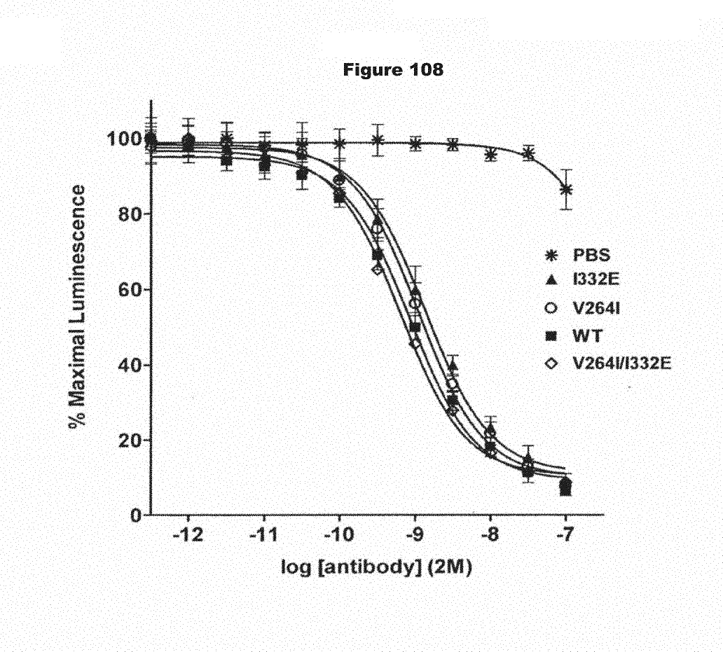

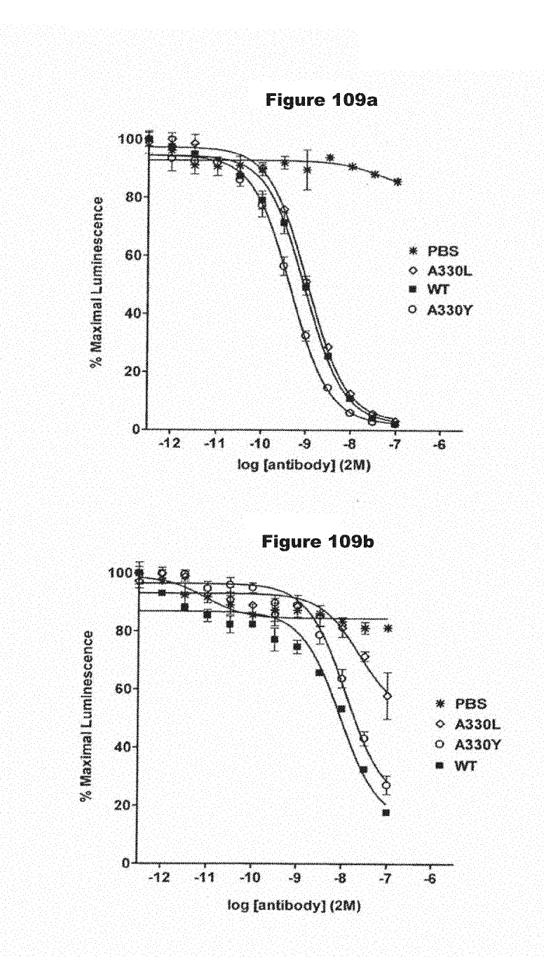

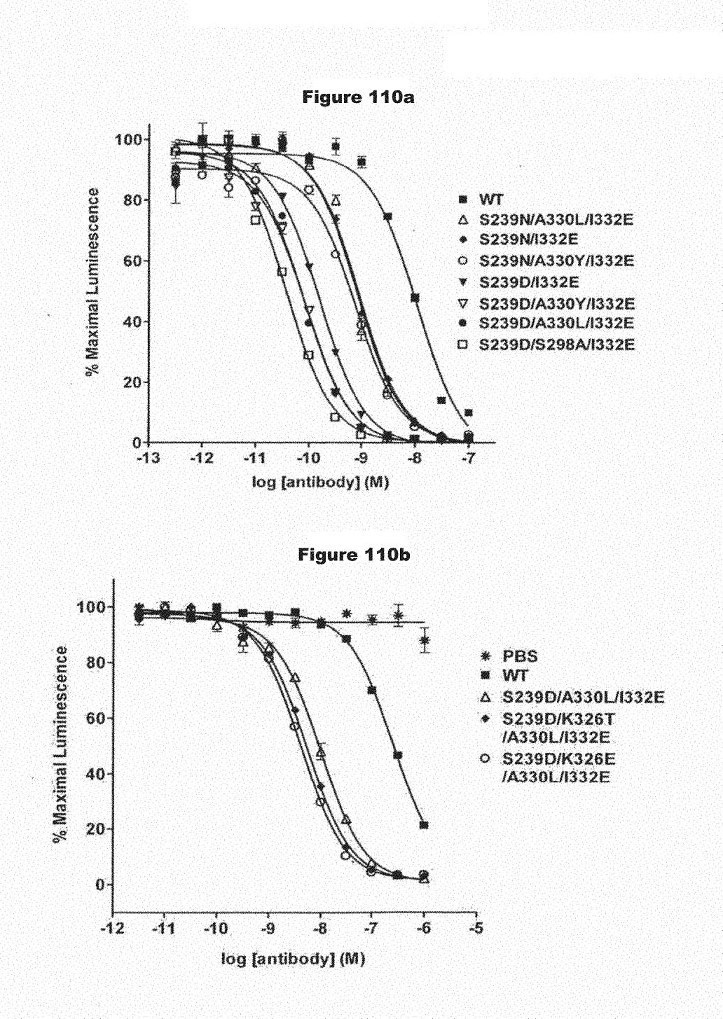

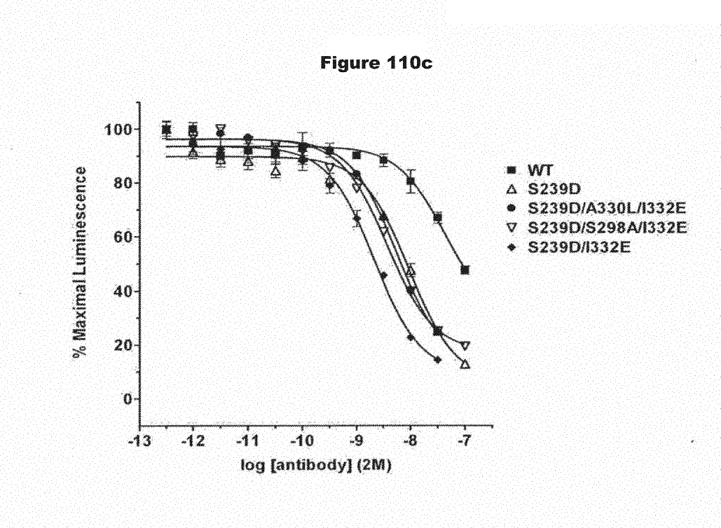

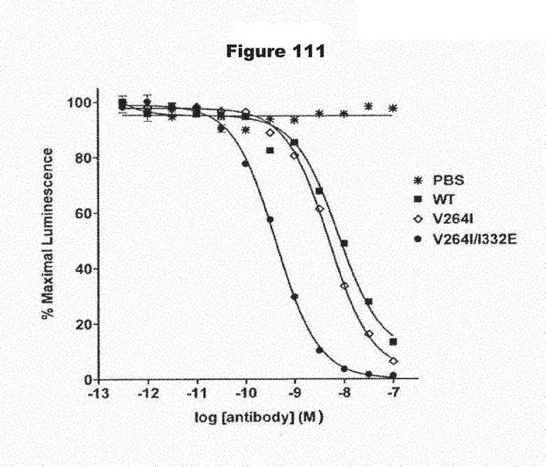

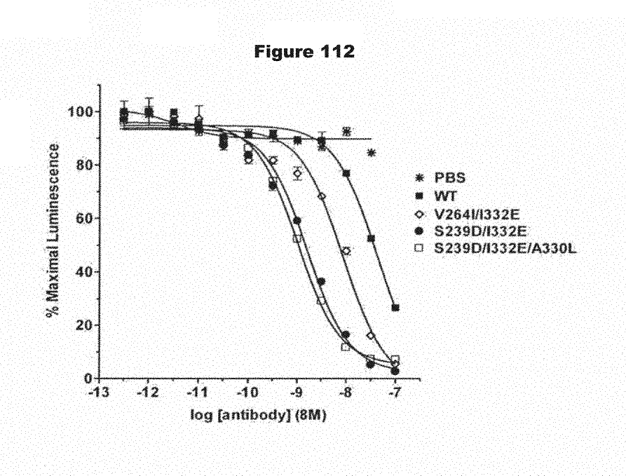

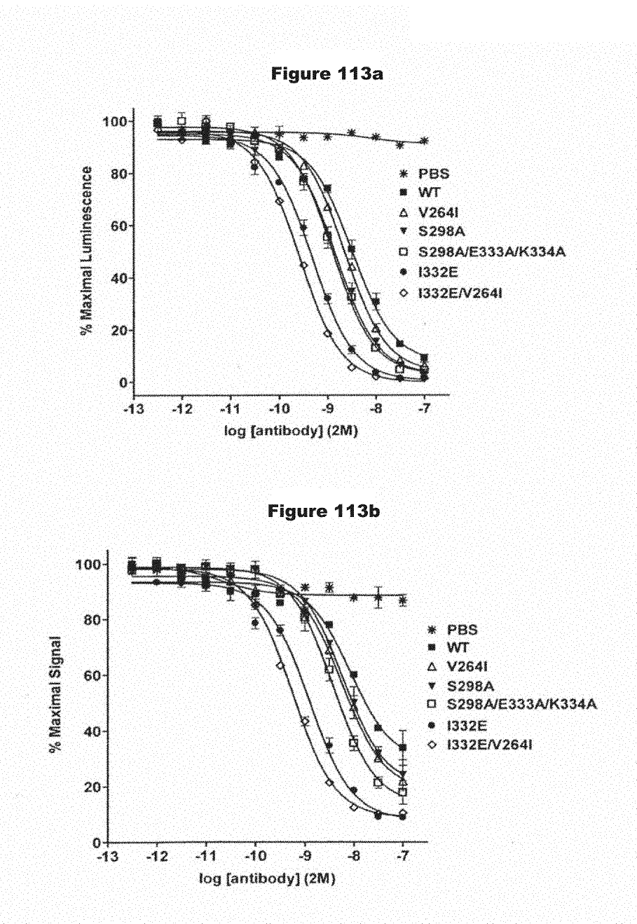

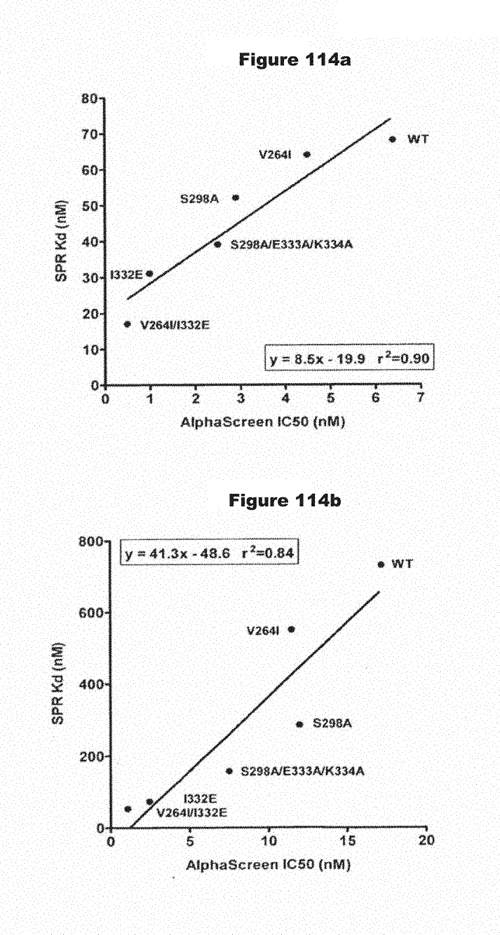

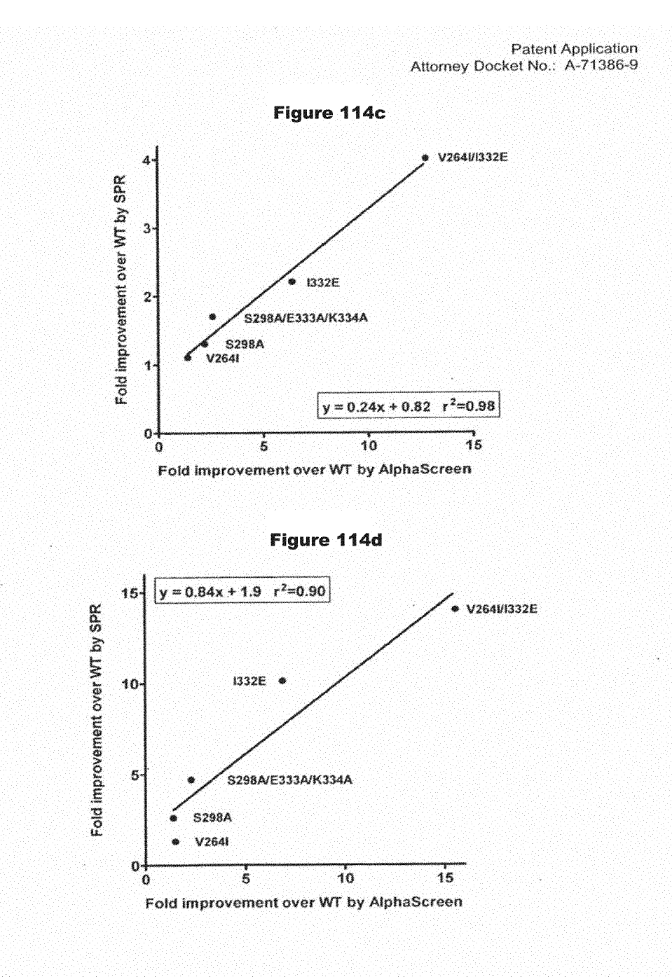

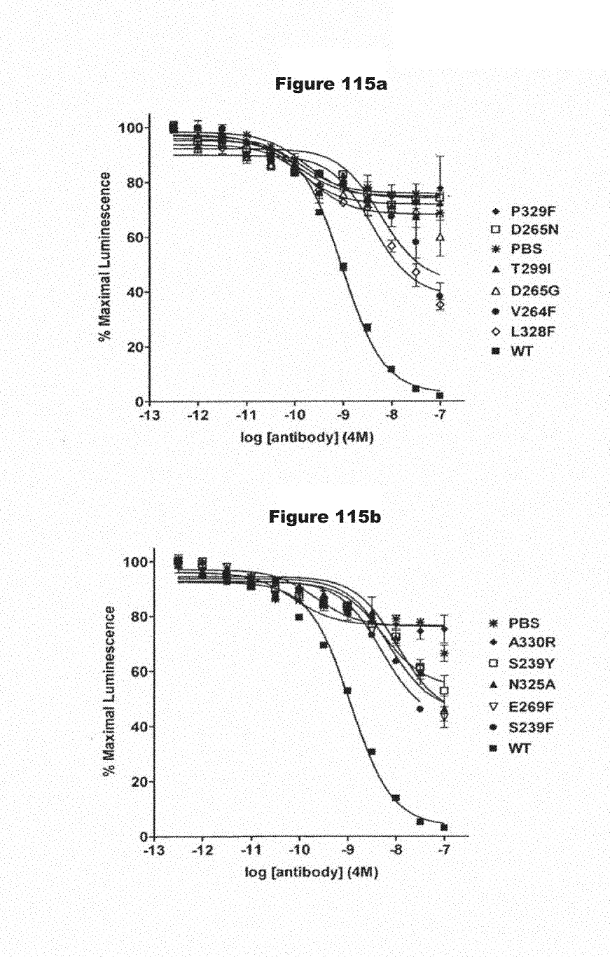

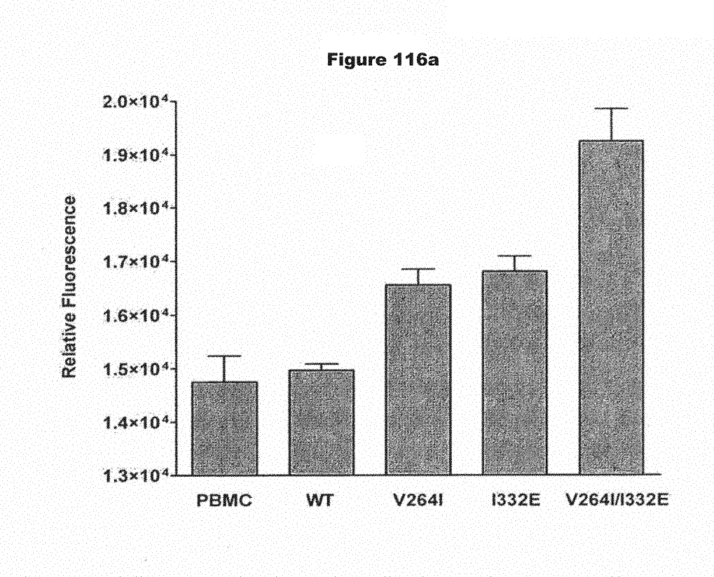

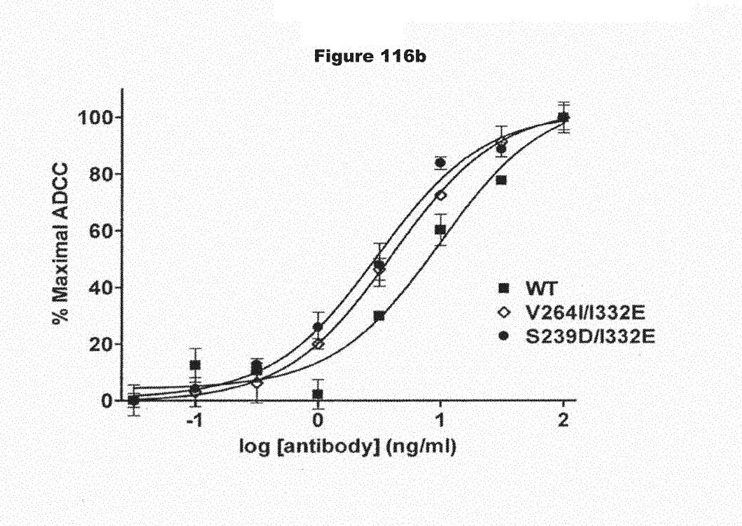

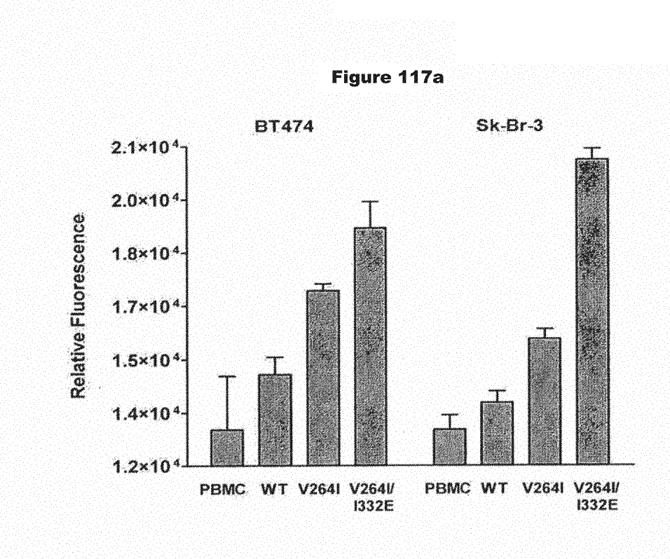

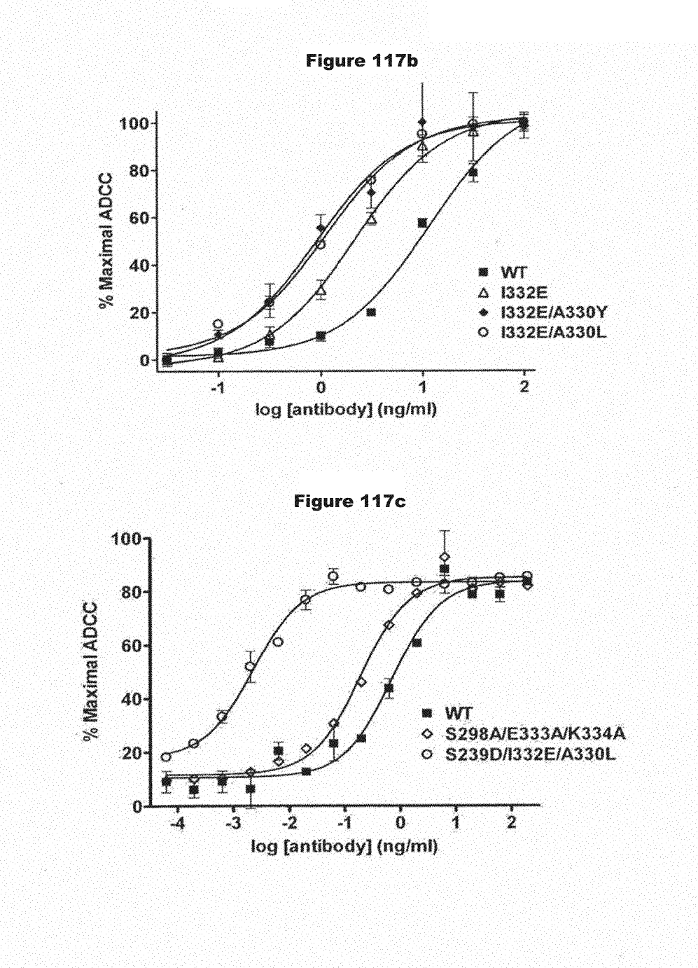

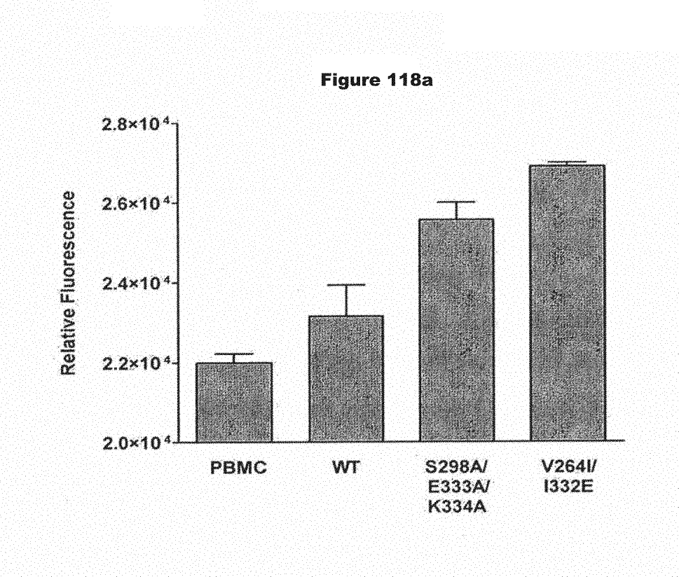

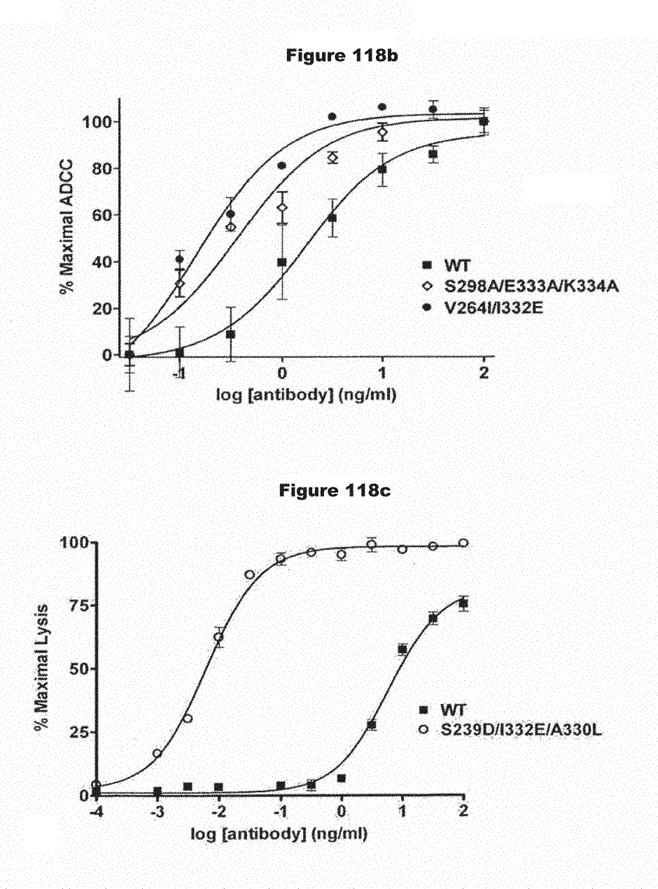

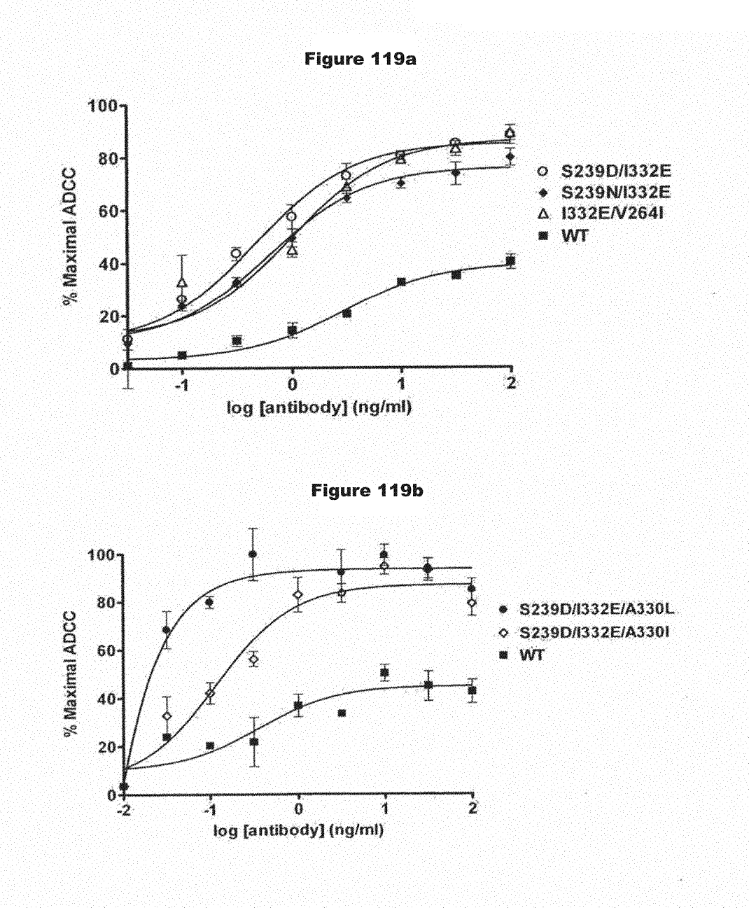

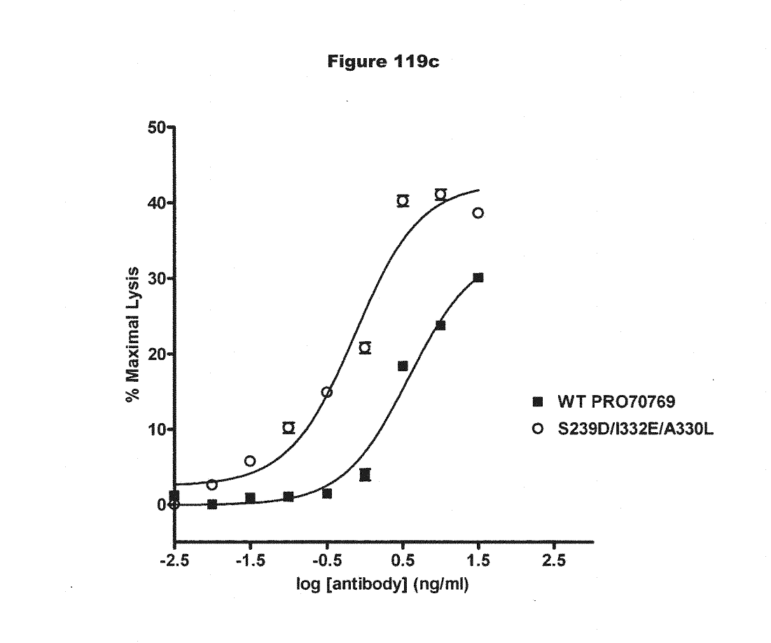

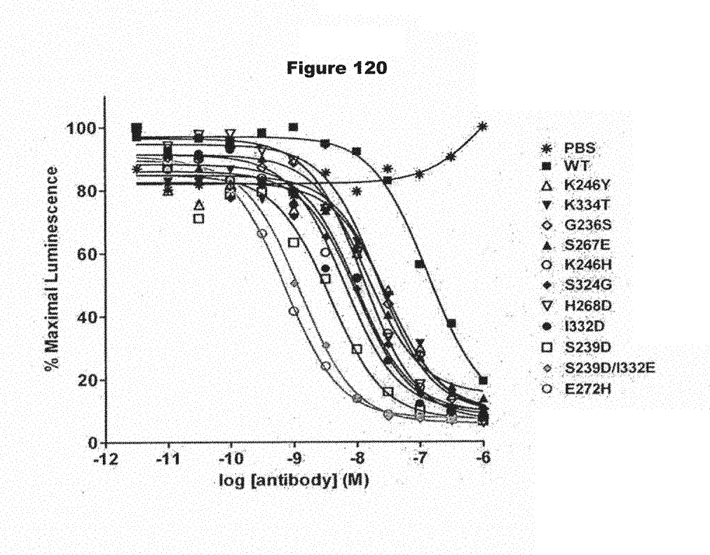

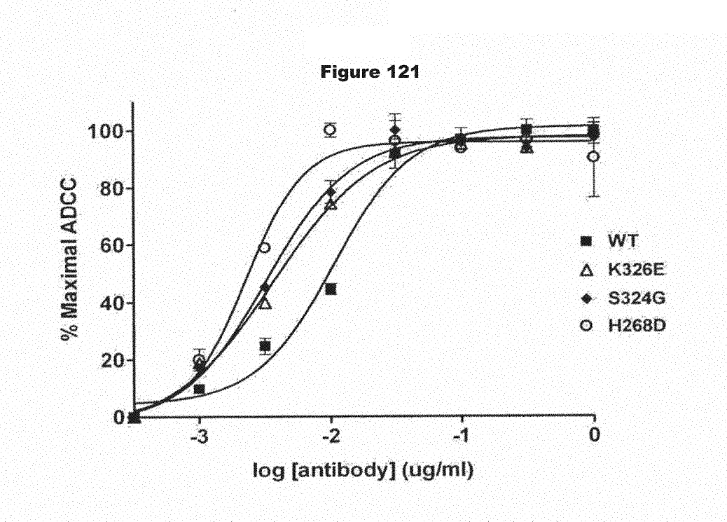

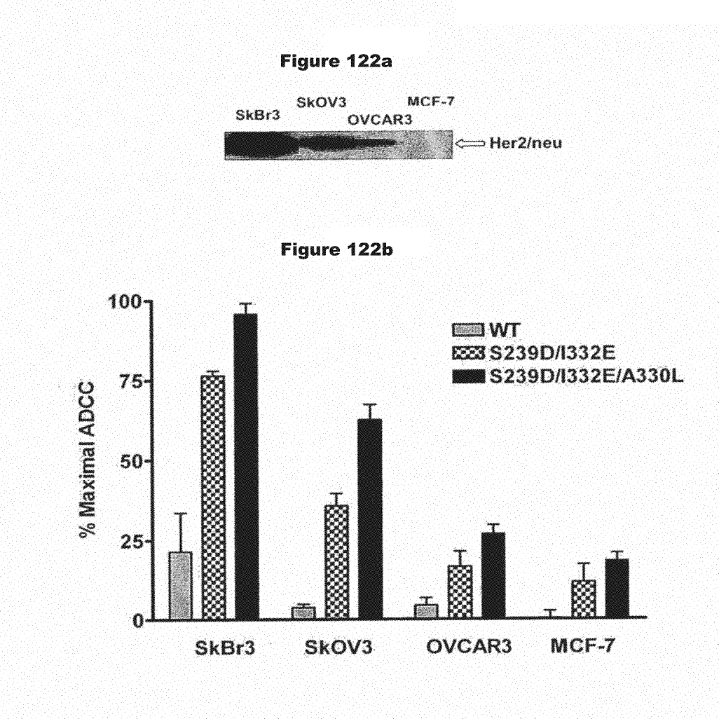

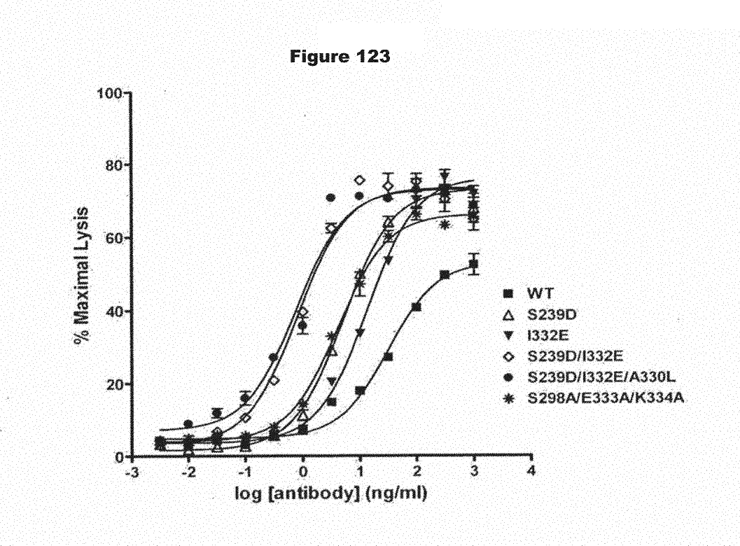

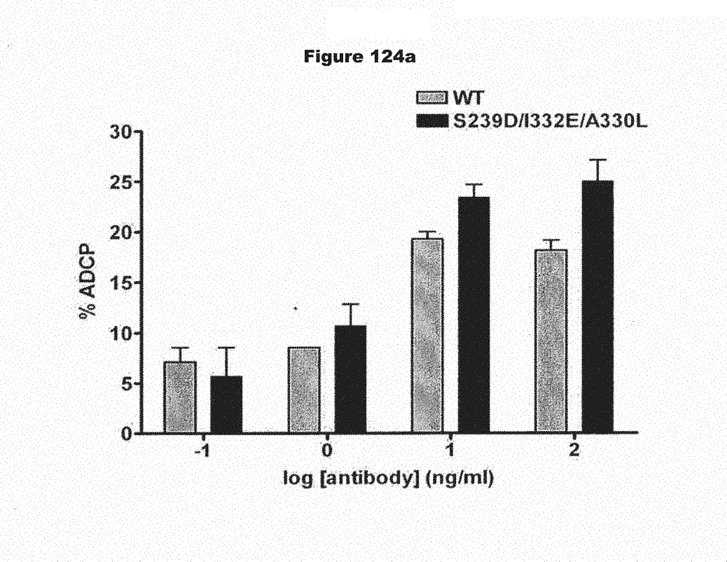

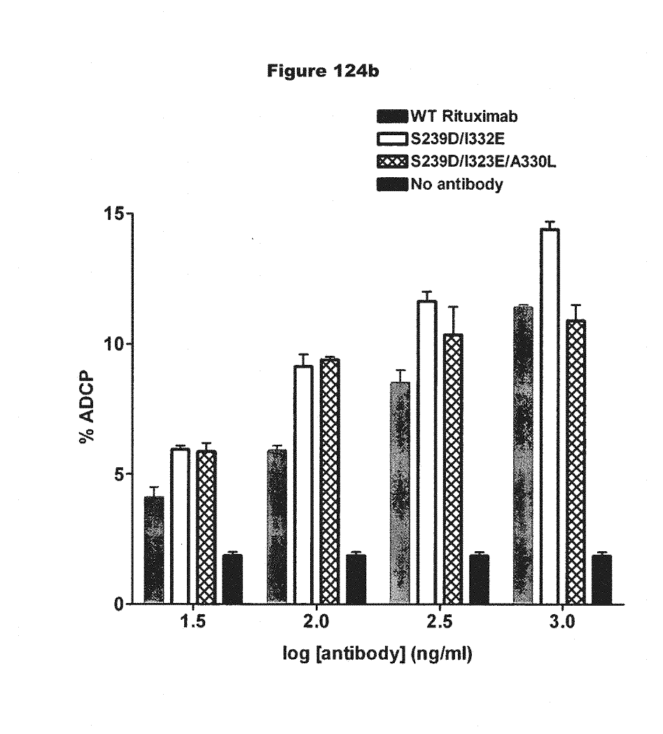

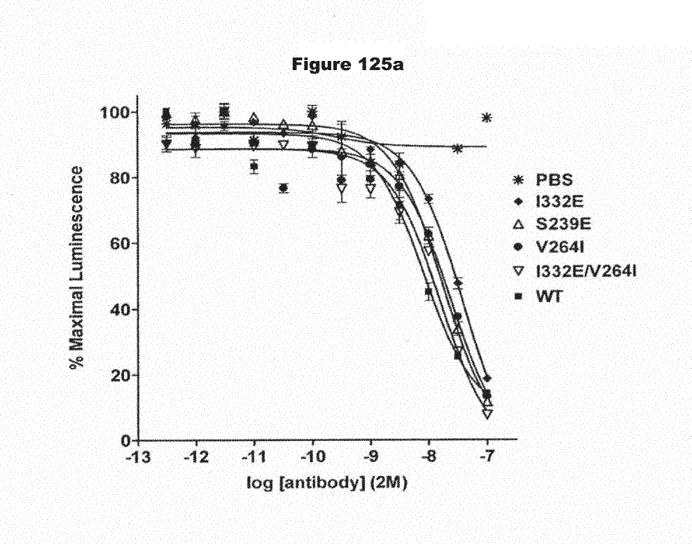

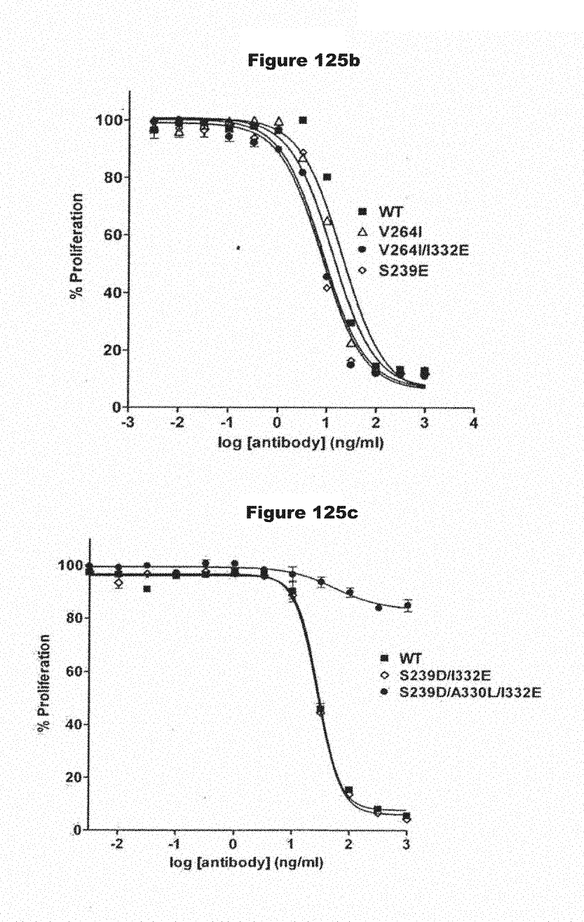

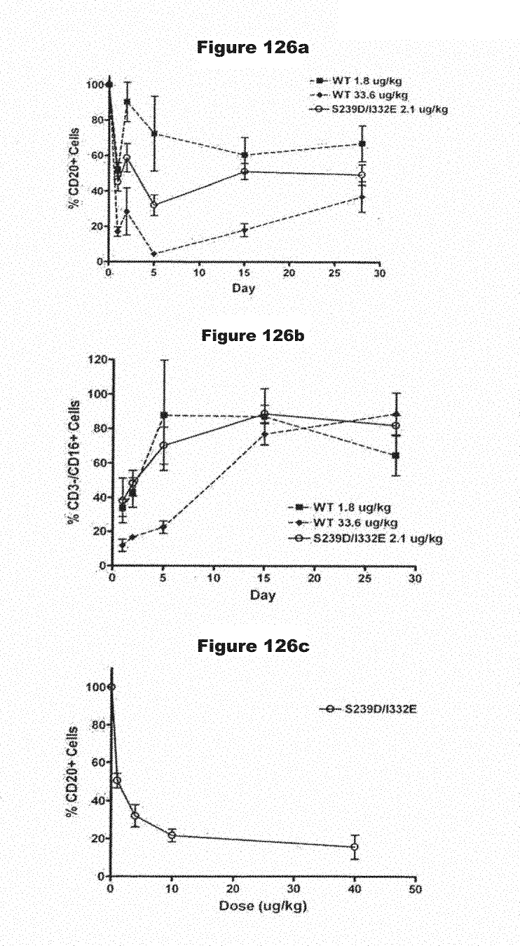

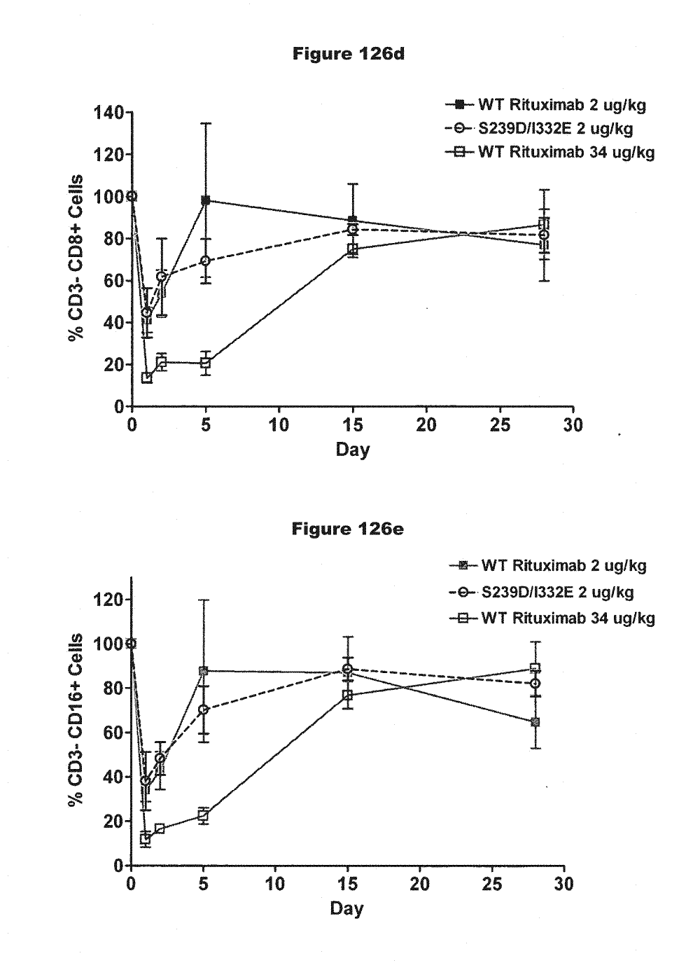

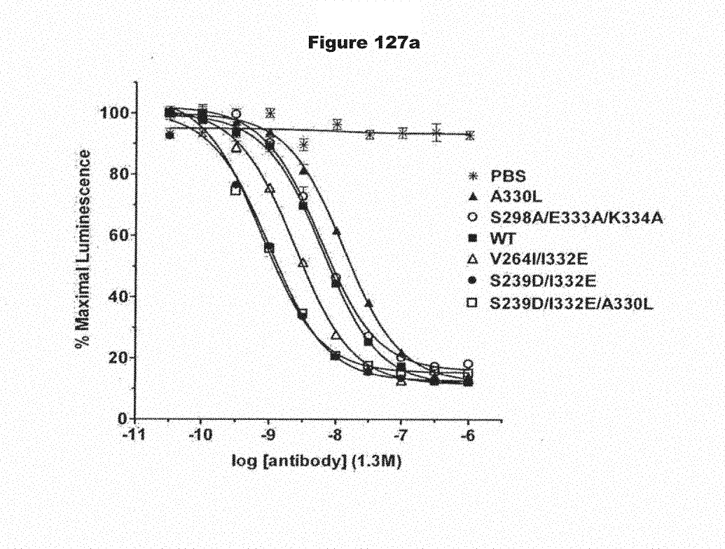

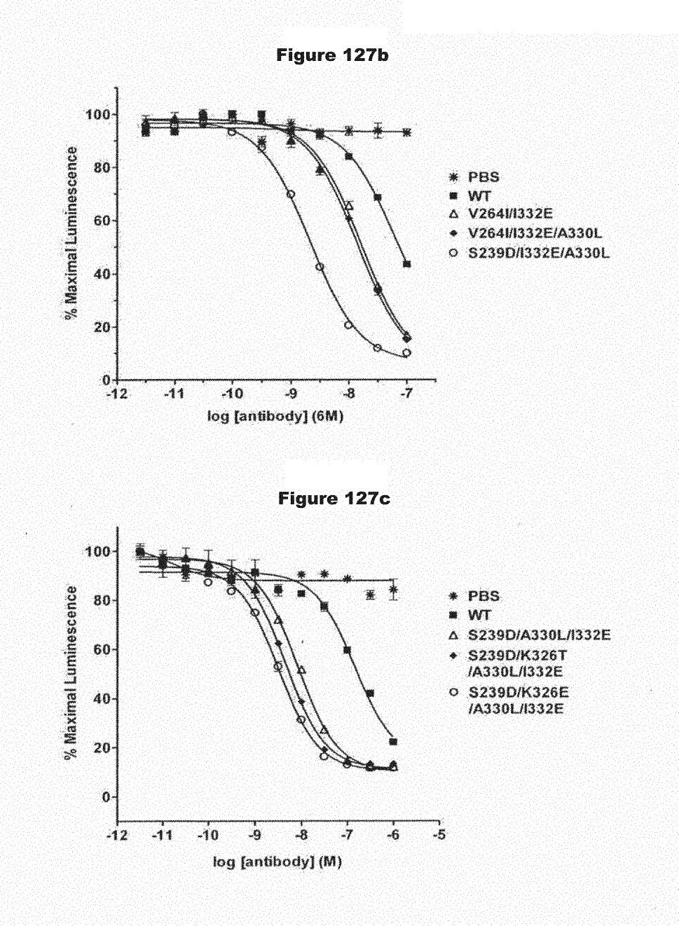

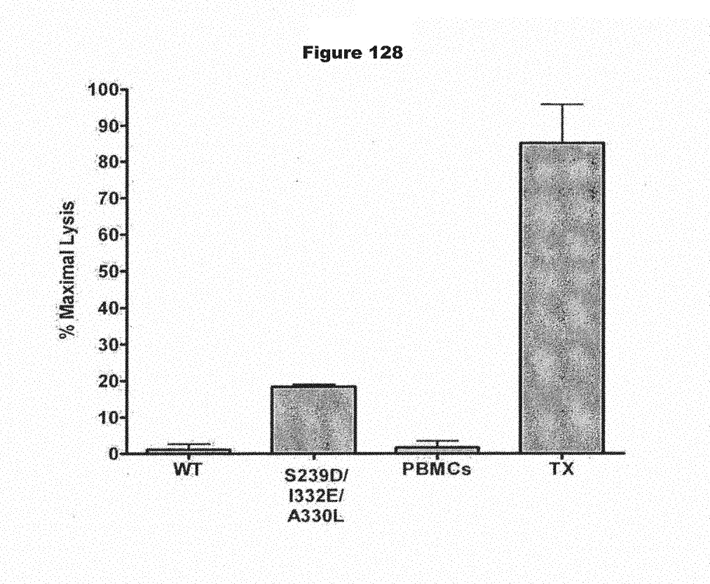

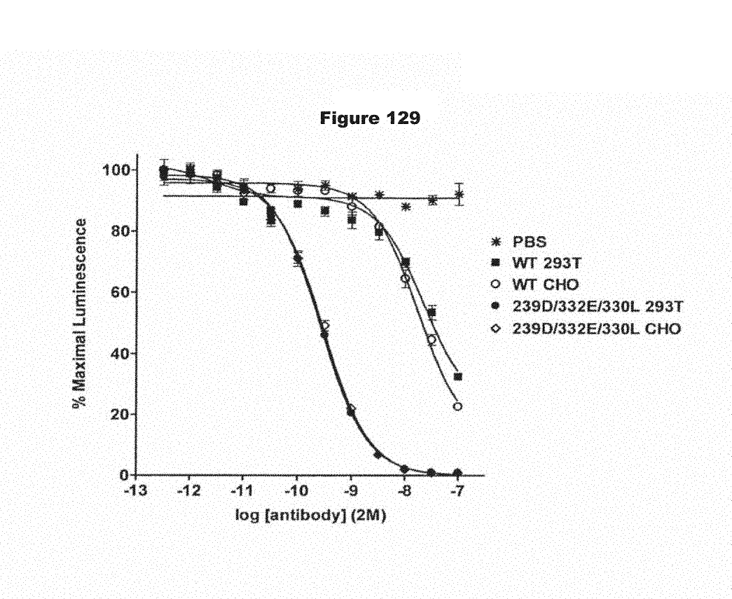

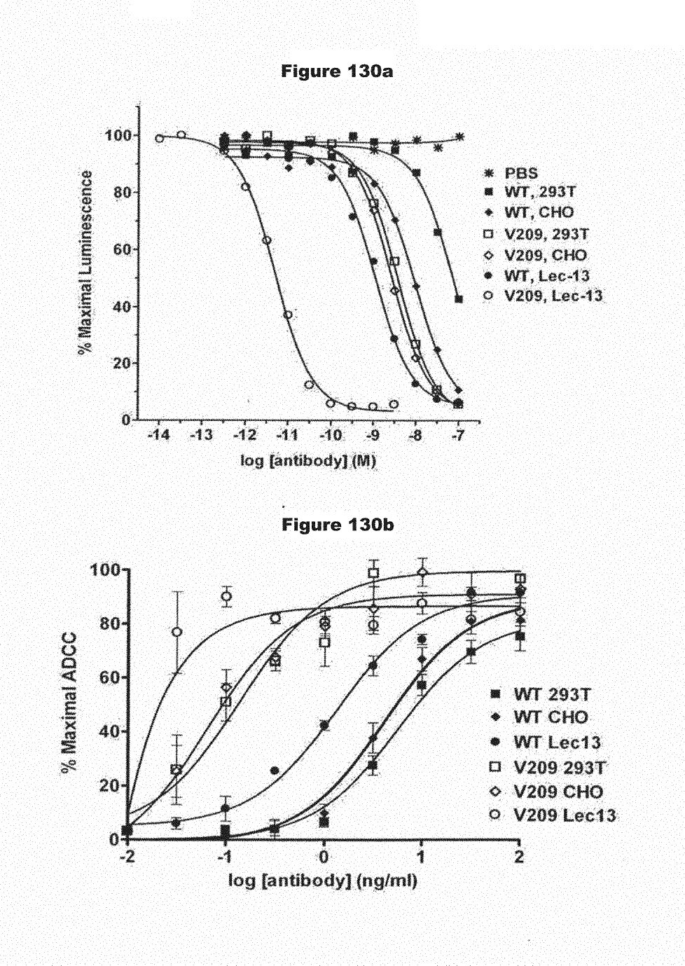

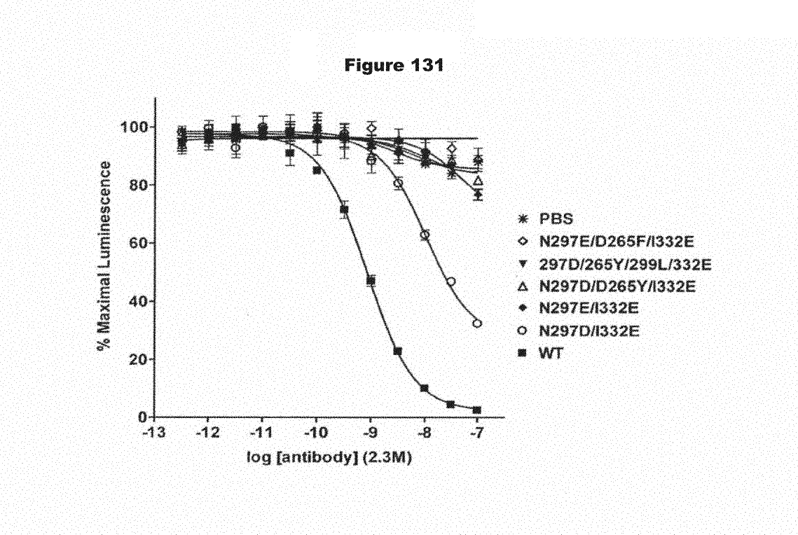

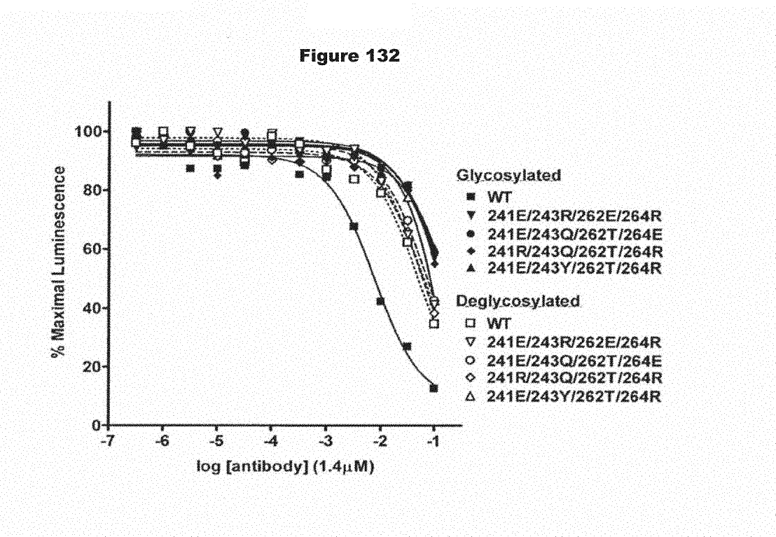

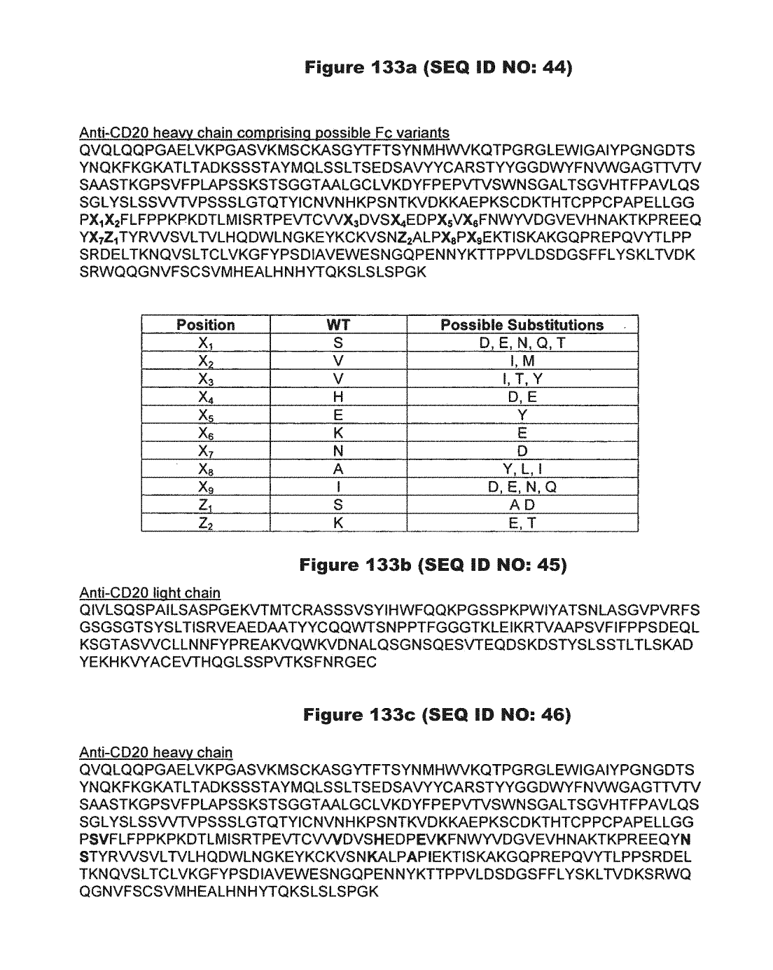

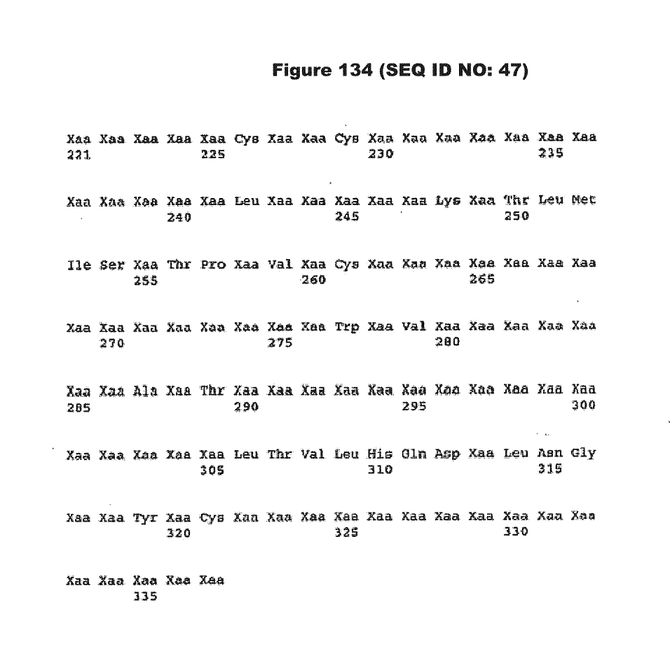

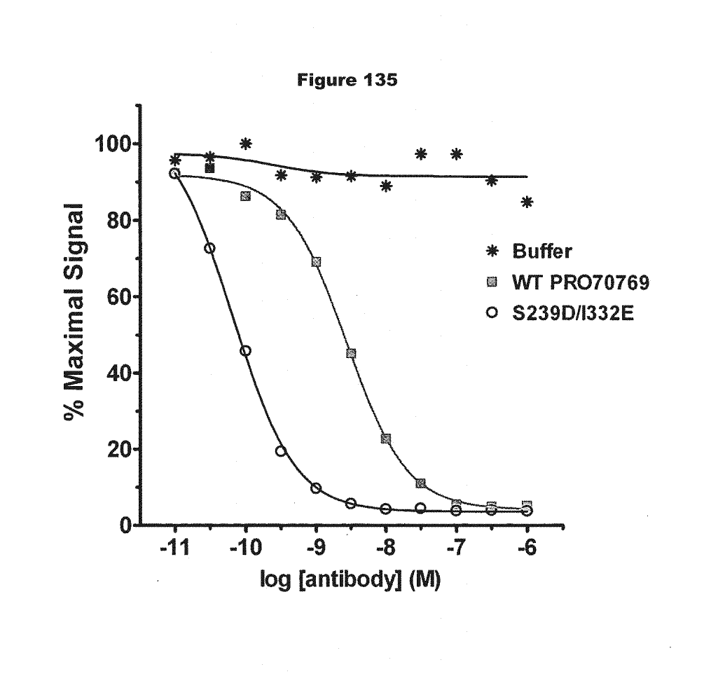

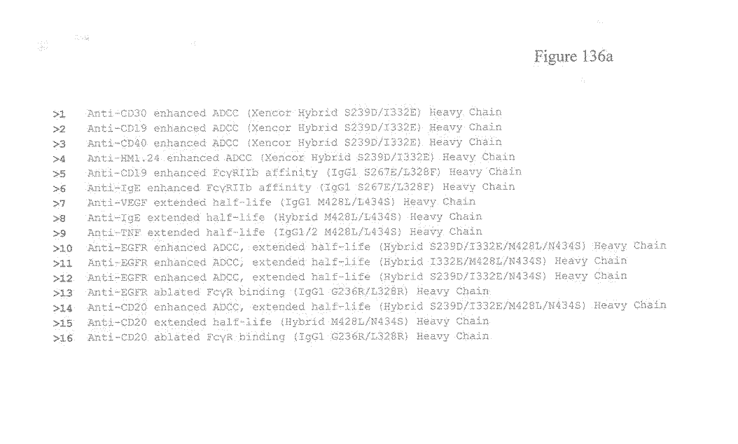

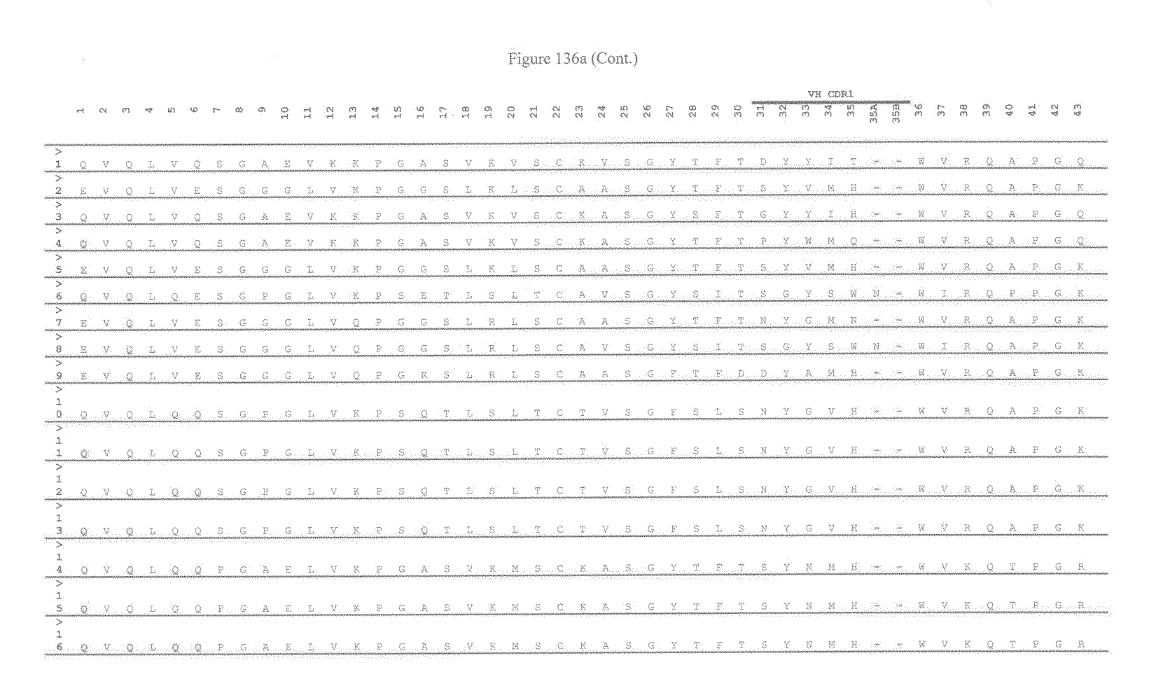

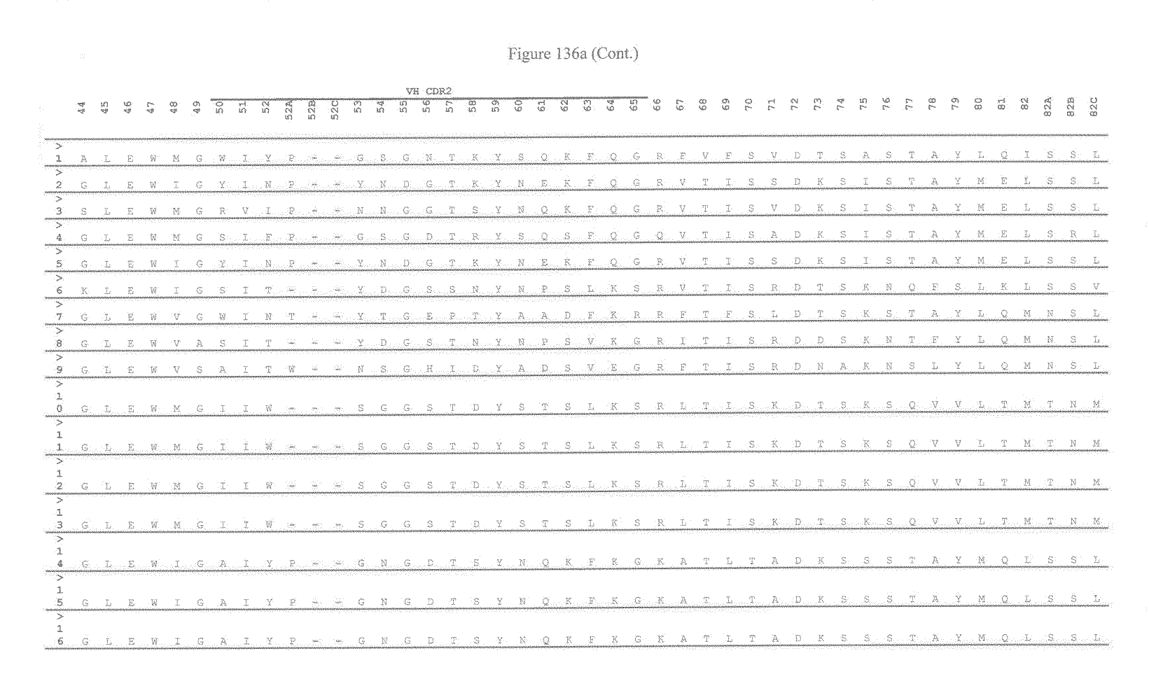

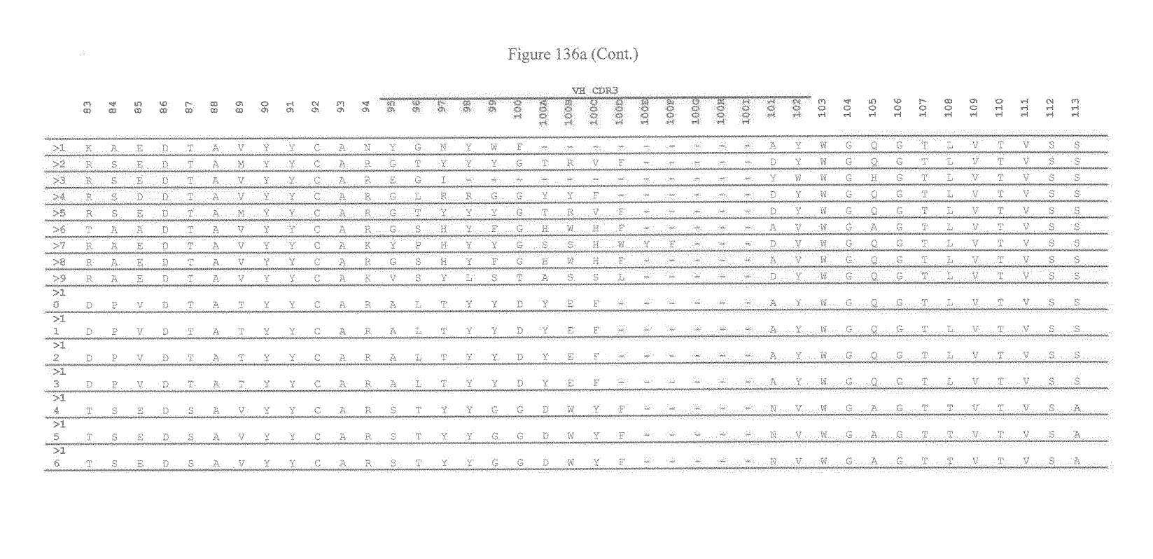

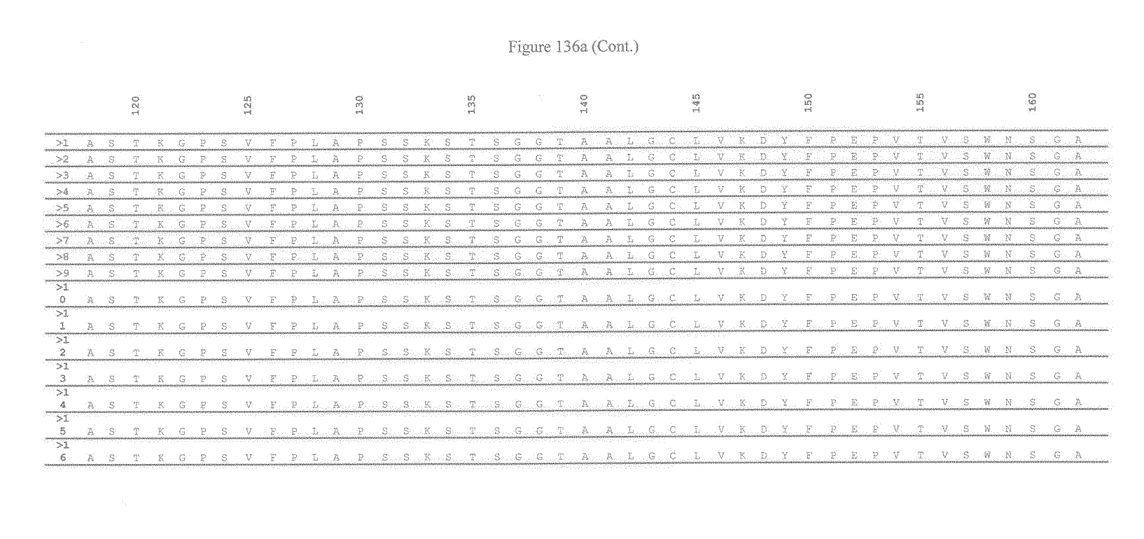

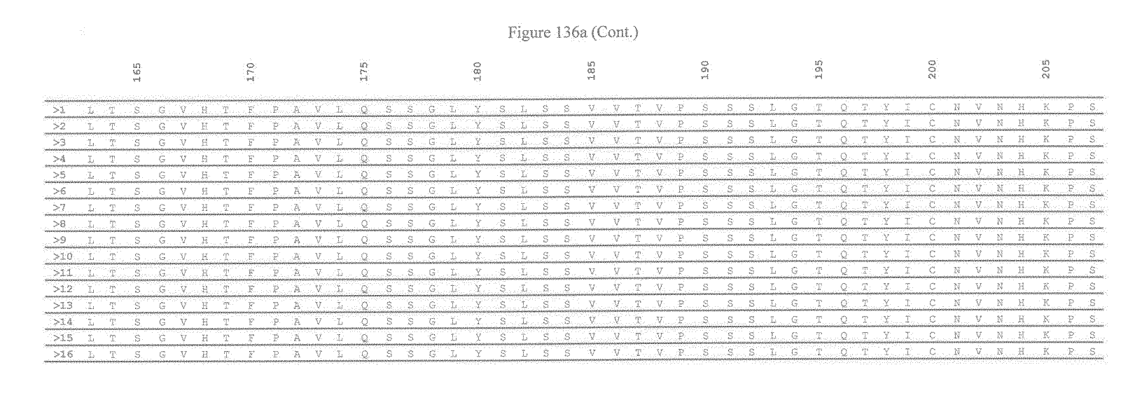

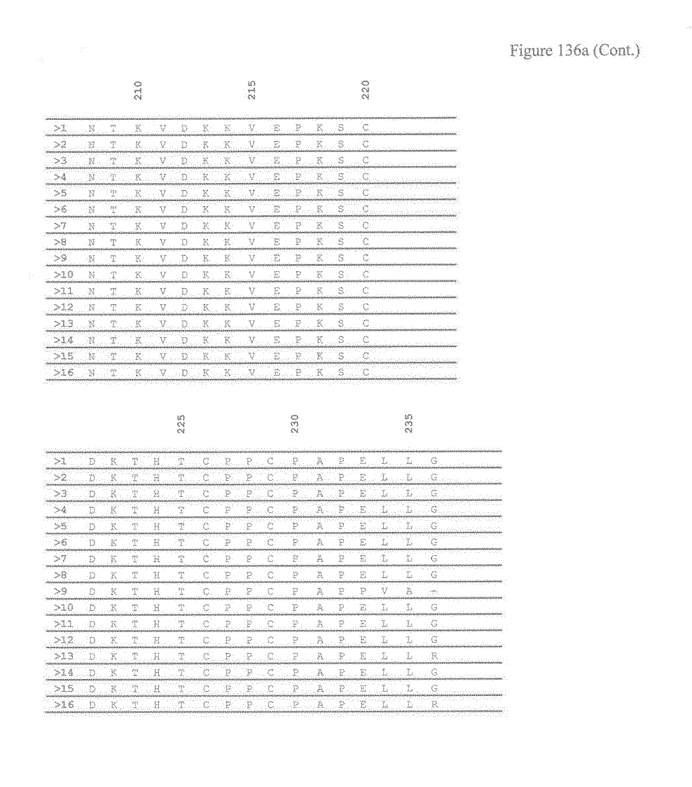

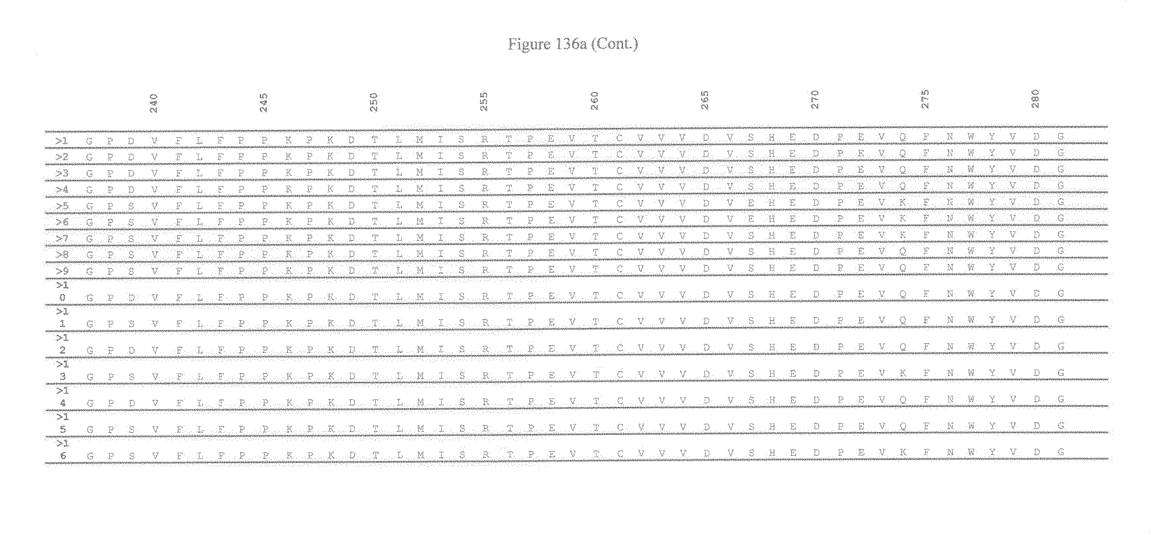

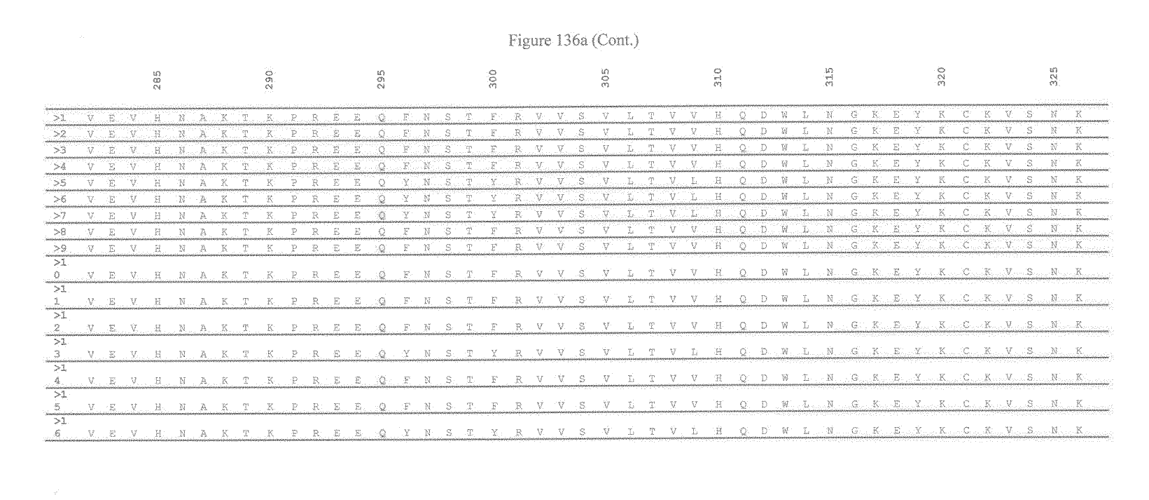

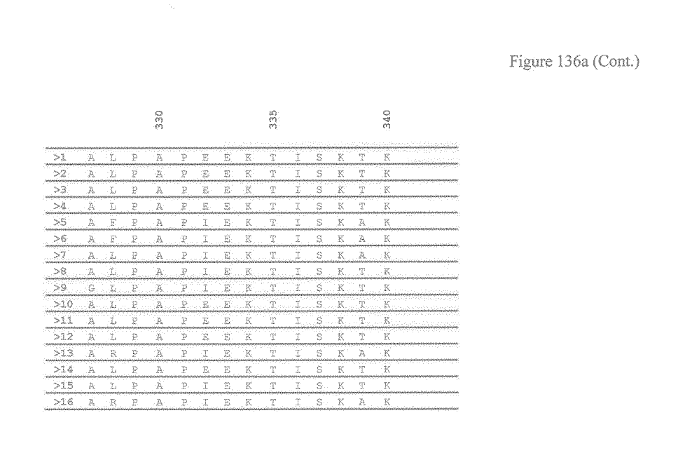

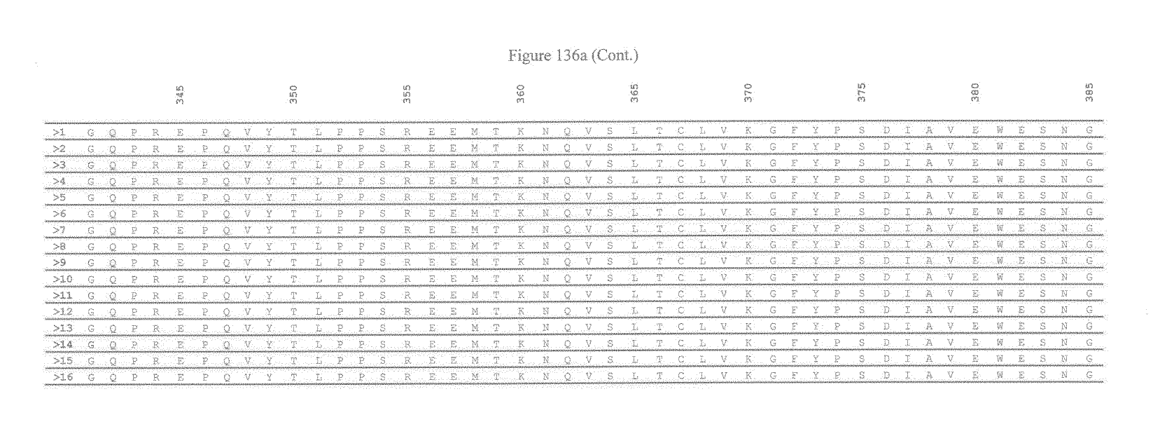

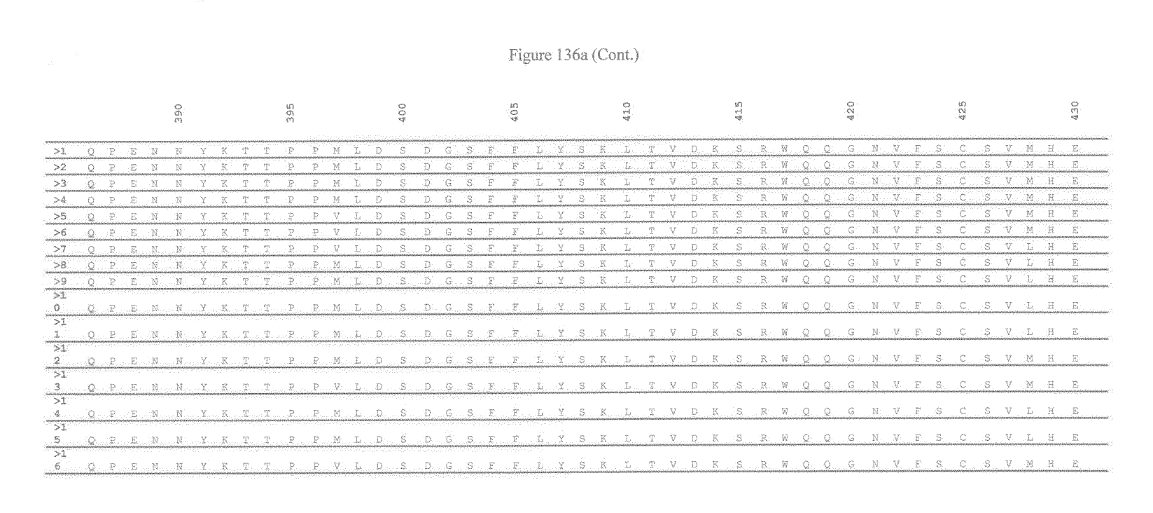

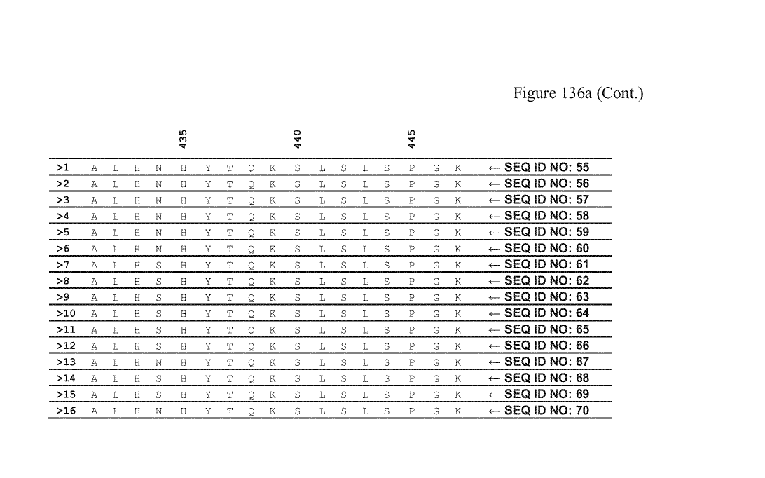

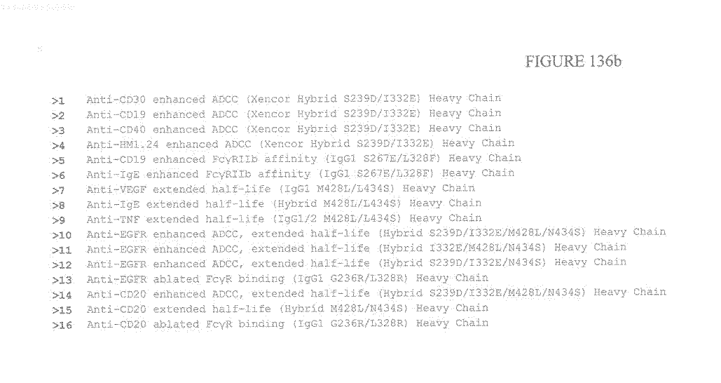

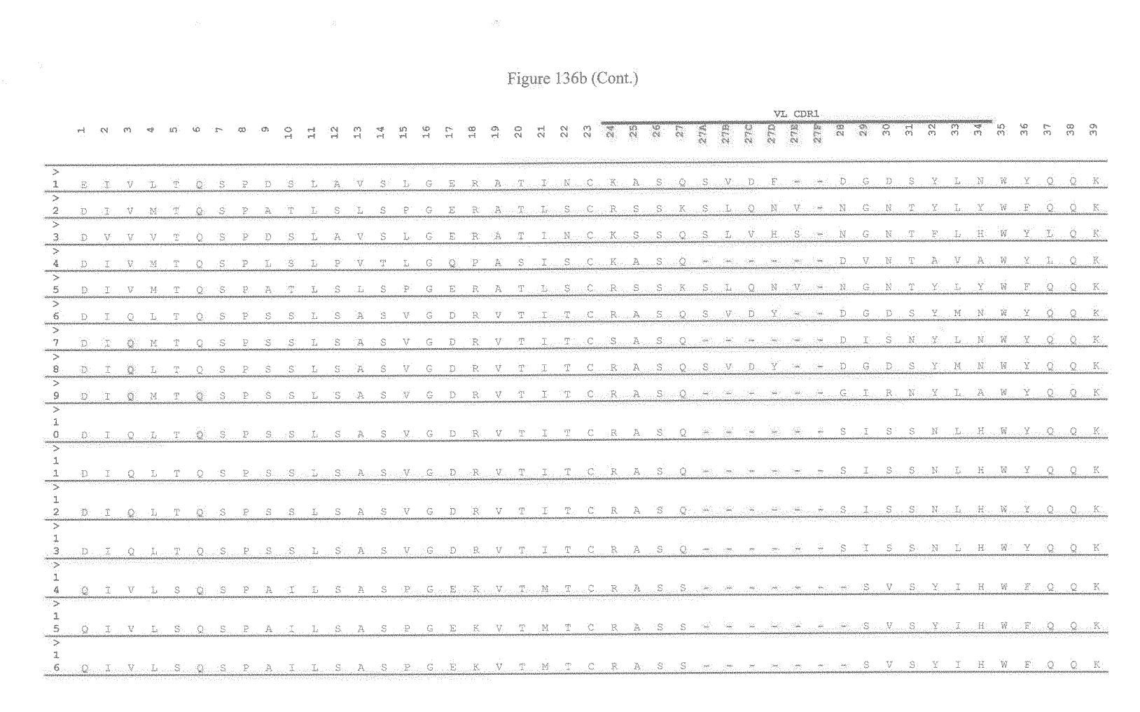

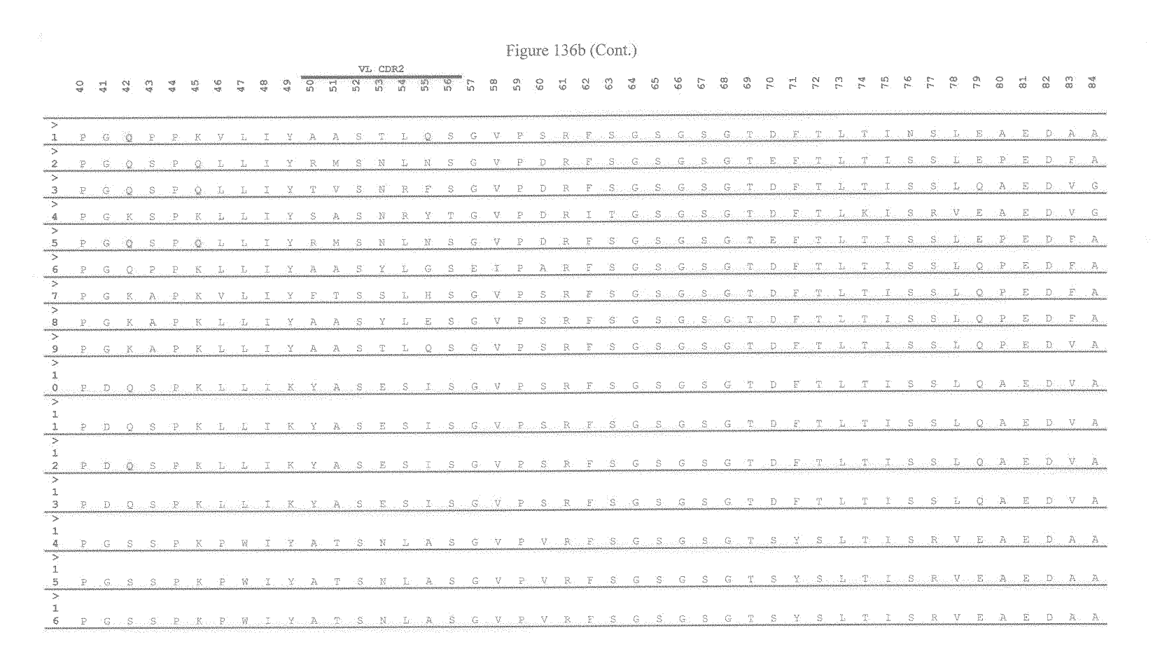

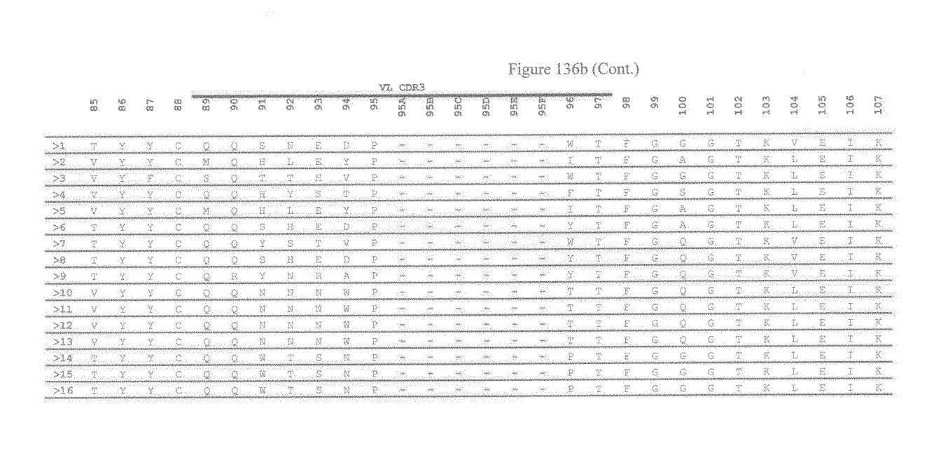

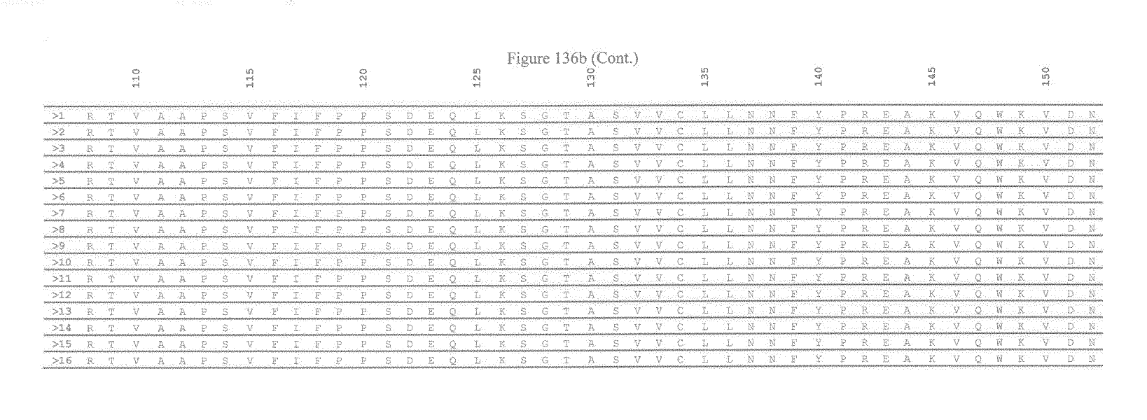

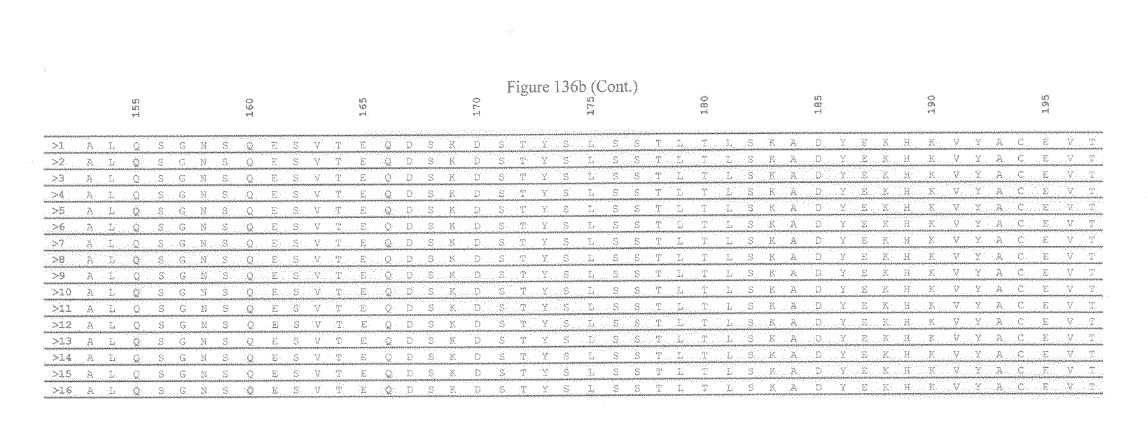

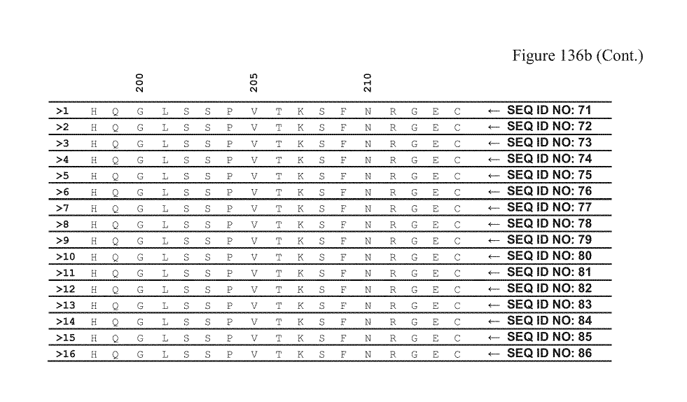

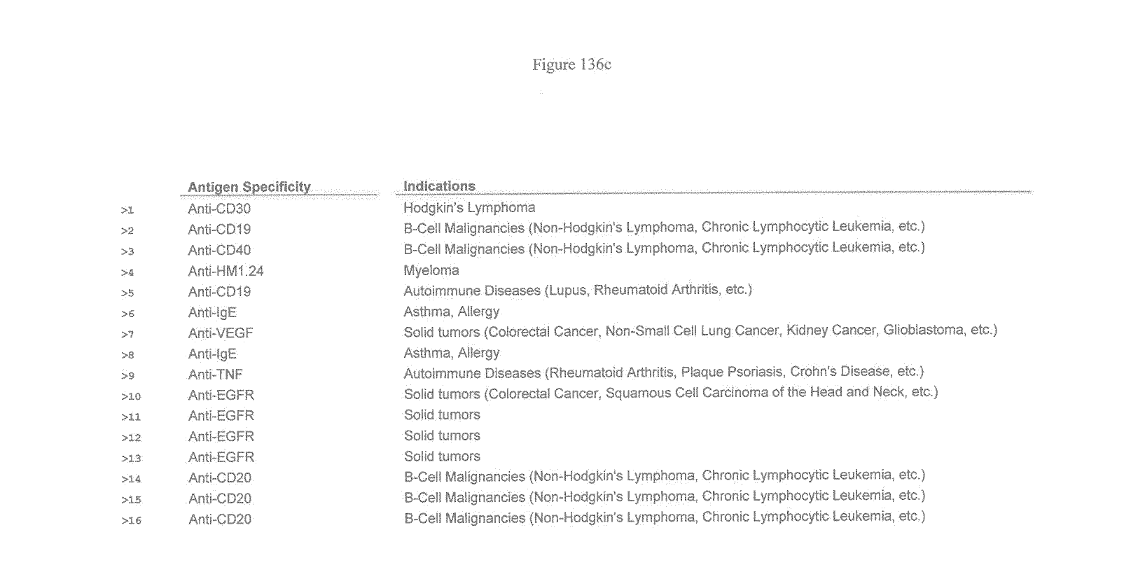

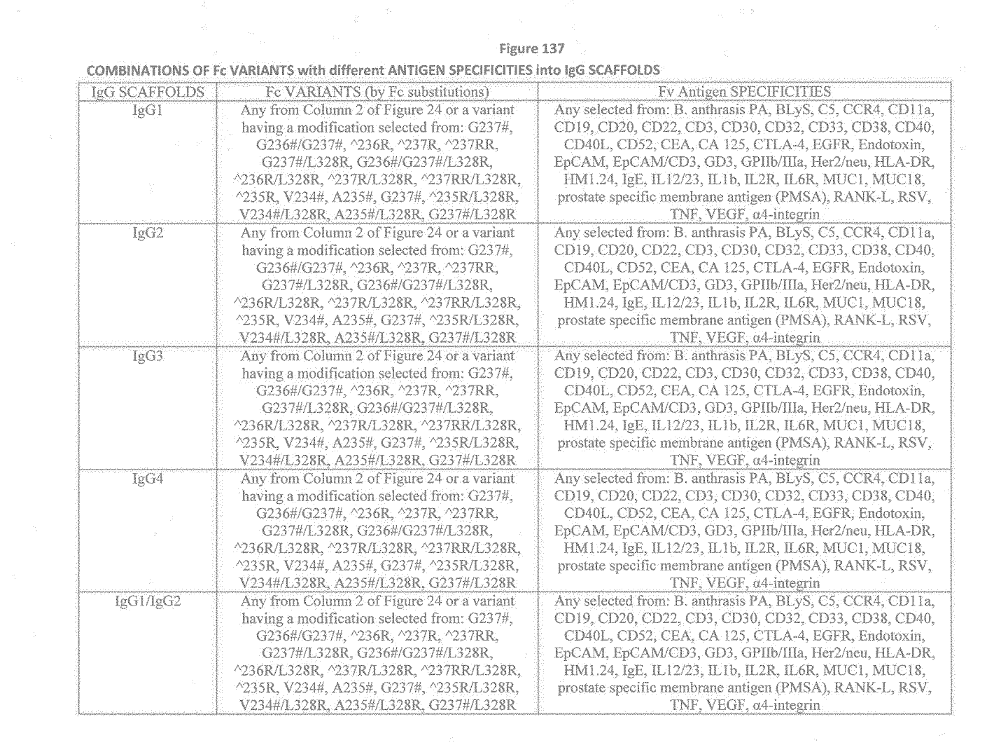

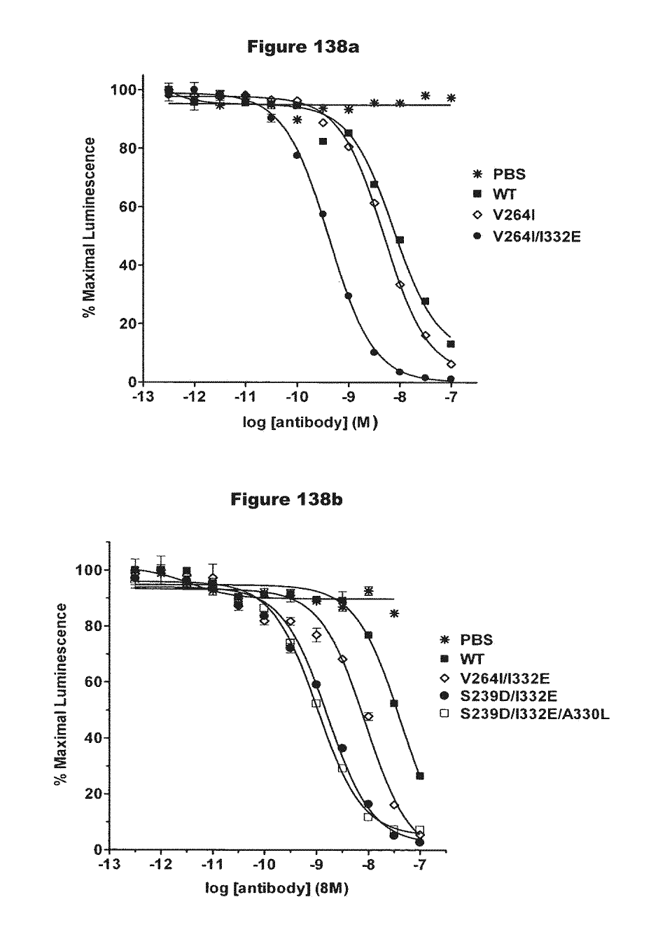

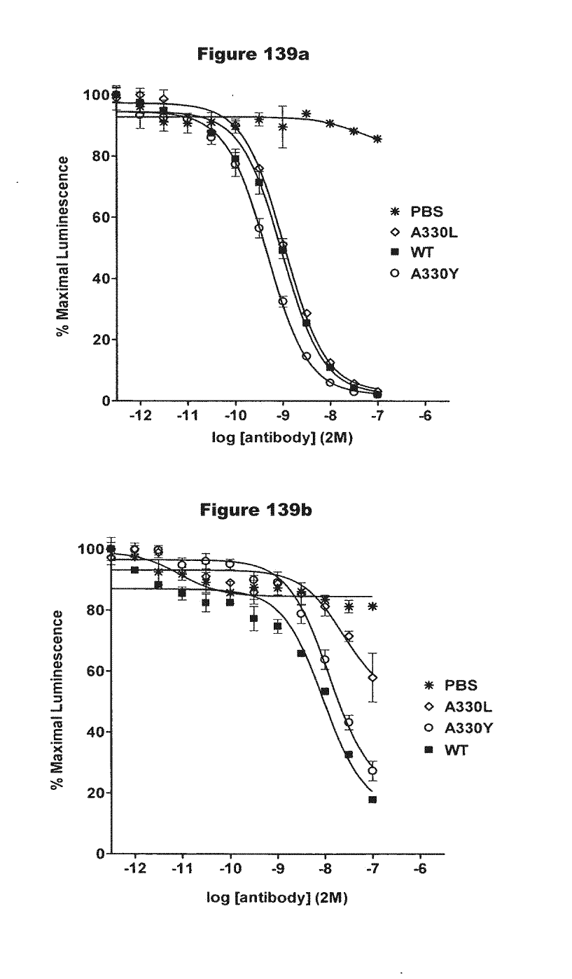

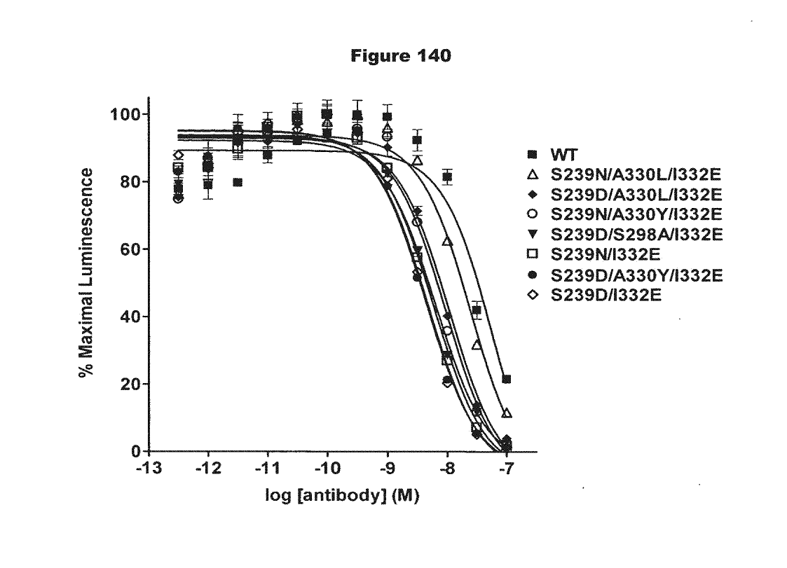

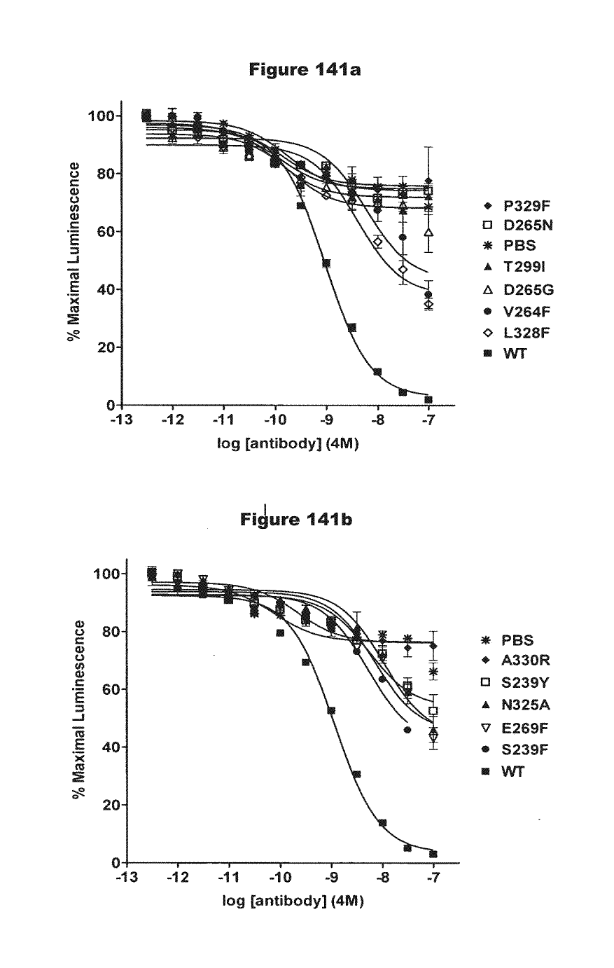

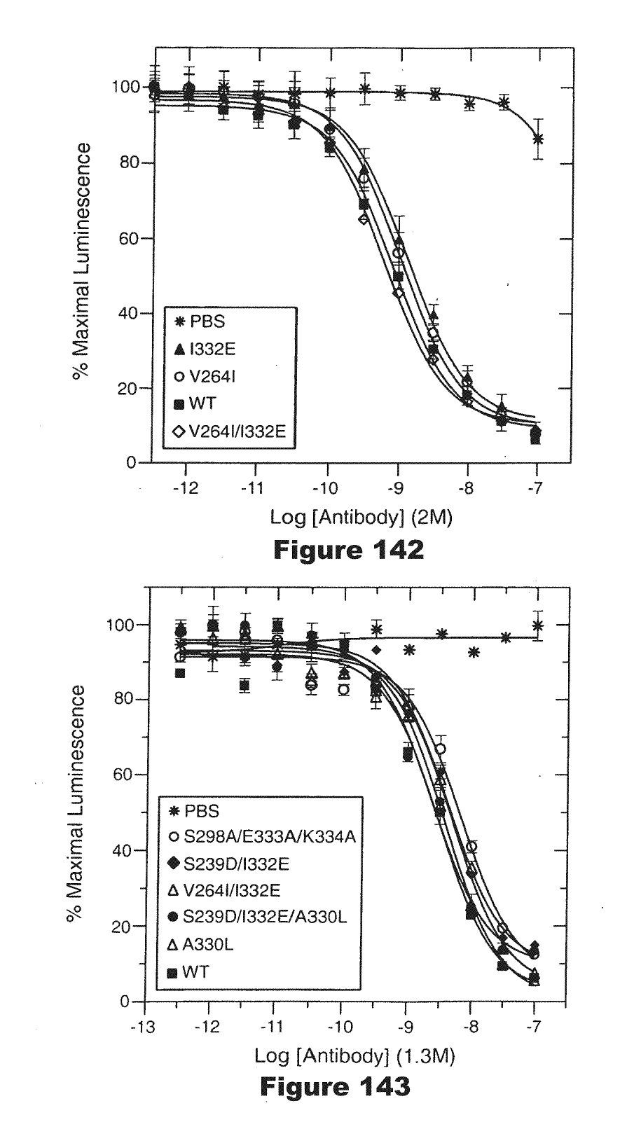

[−log(K_{D})FcγRIIa]−[−log(K_{D})FcγRIIb]. Black represents logarithmic affinity difference between R131 FcγRIIa and FcγRIIb, and gray represents the logarithmic affinity difference between H131 FcγRIIa and FcγRIIb. The bottom graph shows the affinity differences between both isoforms of FcγRIIIa and FcγRIIb, represented mathematically as [−log(K_{D})FcγRIIIa]−[−log(K_{D})FcγRIIb]. Black represents logarithmic affinity difference between V158 FcγRIIIa and FcγRIIb, and gray represents the logarithmic affinity difference between F158 FcγRIIIa and FcγRIIb. FIG. 61b provides the fold affinity improvement of each variant for FcγRIIa and FcγRIIIa relative to the fold affinity improvement to FcγRIIb. Here RIIa represents R131FcγRIIa, HIIa represents H131 FcγRIIa, VIIIa represents V158 FcγRIIIa, FIIIa represents F158 FcγRIIIa, and IIb represents FcγRIIb. As an example, for the R131 isoform of FcγRIIa this quantity is represented mathematically as Fold(KD)_{RIIa}: Fold(KD)_{IIb} or Fold(KD)_{RIIa}/Fold(KD)_{IIb}. See the Examples for a mathematical description of these quantities. FIG. 61c provides a plot of these data.

FIG. 62. Cell-based ADCC assays of anti-epCAM Fc variants. FIG. 62 shows the data for select Fc variant antibodies. The G236A variant mediates reduced ADCC relative to WT, due likely to its reduced affinity for FcγRIIIa and/or FcγRI. ADCC in PBMCs is potentially dominated by NK cells, which express only FcγRIIIa, although in some cases they can express FcγRIIc. Thus, the reduced ADCC of the G236A single variant is consistent with its reduced affinity for this receptor. However, combination of the G236A substitution with modifications that improve affinity for these activating receptors, for example including but not limited to substitutions at 332 and 239, provide substantially improved ADCC relative to the parent WT antibody.

FIG. 63a. Receptor expression density of FcγRI (CD64), FcγRIIa and FcγRIIb (CD32), and FcγRIIIa (CD16) on monocyte-derived macrophages. FIG. 63a shows that the monocyte-derived macrophages (MDM) express high levels of FcγRII (99%) and FcγRIII (81%), and moderate (45%) levels of FcγRI. The inability to distinguish between FcγRIIa and FcγRIIb is due to the unavailability of commercial antibodies that selectively bind these two receptors.

FIG. 63b-63c. Cell-based ADCP assay of anti-epCAM Fc variants. FIG. 63b shows the results of an ADCP assay of select anti-EpCAM Fc variants in the presence of macrophages. FIG. 63c show a repeat experiment with some of these variants. The data show that the improved FcγRII:FcγRIIb profile of the I332E/G236A variant relative to the I332E single variant provides enhanced phagocytosis. Interestingly, G236A does not improve phagocytosis of the S239D/I332E variant.

FIG. 64a-64b. Cell-based DC activation assay of anti-EpCAM Fc variants. FIG. 64a shows the quantitated receptor expression density on monocyte-derived dendritic cells measured with antibodies against FcγRI (CD64), FcγRIIa and FcγRIIb (CD32), and FcγRIIIa (CD16) using flow cytometry. “Control” indicates no antibody was used and is a negative control. The diagrams show the percentage of cells labeled with PE-conjugated antibody against the indicated FcγR. FIG. 64b shows the dose-dependent TNFa release by dendritic cells in the presence of WT and Fc variant antibodies and EpCAM+LS180 target cells. The IgG1 negative control binds RSV and not EpCAM, and thus does not bind to the target cells.

FIG. 65a-65c. Binding of Fc variant antibodies comprising substitutions 298A, 326A, 333A, and 334A to human V158 FcγRIIIa, F158 FcγRIIIa, and FcγRIIb as measured by competition AlphaScreen assay. FIG. 65a shows the legend for the data. Antibodies in FIG. 65b comprise the variable region of the anti-CD52 antibody alemtuzumab (Hale et al., 1990, Tissue Antigens 35:118-127; Hale, 1995, Immunotechnology 1:175-187), and antibodies in FIG. 65c comprise the variable region of the anti-CD20 PRO70769 (PCT/US2003/040426).

FIG. 66. Preferred positions and substitutions of the invention that may be used to engineer Fc variants with selective FcγR affinity.

FIG. 67. Affinity data for binding of 293T-expressed (fucosylated) and Lec13-expressed (defucosylated) anti-EpCAM antibodies to human FcγRI, R131 and H131 FcγRIIa, FcγRIIb, and V158 FcγRIIIa as determined by SPR. Provided are the equilibrium dissociation constant (K_{D}), the Fold K_{D} relative to WT, and the negative log of the K_{D} (−log(K_{D}). n. d.=not determined.

FIG. 68. Plot of the negative log of the K_{D} for binding of 293T-expressed (fucosylated) and Lec13-expressed (defucosylated) anti-EpCAM antibodies to human FcγRI, R131 FcγRIIa, H131 FcγRIIa, FcγRIIb, and V158 FcγRIIIa. *=the data for binding of WT IgG1 defucosylated to FcγRIIb was not determined due to insufficiency of sample.

FIG. 69. Binding of select anti-CD30 Fc variants to human V158 FcγRIIIa as measured by competition AlphaScreen assay.

FIG. 70. Summary of V158 FcγRIIIa binding properties of anti-CD30 Fc variants. Shown are the Fold-IC50s relative to WT as determined by competition AlphaScreen.

FIG. 71a-71b. Differences between human and mouse FcγR biology. FIG. 71a shows the putative expression patterns of different FcγRs on various effector cell types “yes” indicates that the receptor is expressed on that cell type. Inhibitory receptors in the human and mouse are shown in gray. FIG. 71b shows the % identity between the human (h) and mouse (m) FcγR extracellular domains. Human receptors are shown in black and mouse receptors are shown in gray.

FIG. 72. Summary of human and mouse anti-EGFR antibodies constructed. For each variant are listed the variable region (Fv), constant light chain (CL), and constant heavy chain (CH).

FIG. 73. Affinity data for binding of human and mouse anti-EGFR Fc variant antibodies to mouse Fc receptors FcγRI, FcγRII (FcγRIIb), FcγRIII, and FcγRIV as determined by SPR. Provided are the equilibrium dissociation constant (K_{D}), the Fold K_{D} relative to WT, and the negative log of the K_{D} (−log(K_{D})) for each variant.

FIG. 74. Plot of the negative log of the K_{D} for binding of human and mouse anti-EGFR Fc variant antibodies to mouse Fc receptors FcγRI, FcγRII (FcγRIIb), FcγRIII, and FcγRIV.

FIG. 75a-75h. Amino acid sequences of variable light (VL) and heavy (VH) chains used in the present invention, including PRO70769 (FIGS. 75a and 75b), H4.40/L3.32 C225 (FIGS. 75c and 75d), H3.77/L3 17-1A (FIGS. 75e and 75f), and H3.69 V2/L3.71 AC10 (FIGS. 75g and 75h).

FIG. 76a-76f Amino acid sequences of mouse constant light kappa (FIG. 76a) and heavy (FIGS. 76b-76f) chains of the present invention.

FIG. 77a-77b. Fc variants and FcγR binding data. All Fc variants were constructed in the context of the antibody PRO70769 IgG1. Fold indicates the fold IC50 relative to WT PRO70769 IgG1 for binding to human V158 and F158 FcγRIIIa as measured by the competition AlphaScreen assay.

FIG. 78a-78b. Binding to human V158 FcγRIIIa (FIGS. 78a) and F158 FcγRIIIa (FIG. 78b) by select PRO70769 Fc variants as determined by the competition AlphaScreen assay. In the presence of competitor antibody (Fc variant or WT) a characteristic inhibition curve is observed as a decrease in luminescence signal. The binding data were normalized to the maximum and minimum luminescence signal for each particular curve, provided by the baselines at low and high antibody concentrations respectively. The curves represent the fits of the data to a one site competition model using nonlinear regression.

FIG. 79a-79b. Binding to human V158 FcγRIIIa and F158 FcγRIIIa by PRO70769 Fc variants as measured by competition AlphaScreen assay. FIG. 79a provides data for select variants, FIG. 79b provides the IC50's and folds relative to WT PRO70769 IgG1.

FIG. 80a-80b. Fc variants and FcγR binding data. All Fc variants were constructed in the context of the variable region PRO70769 and either human IgG1 or IgG(1/2) ELLGG. FIG. 80a provides the IC50's and fold IC50's relative to WT PRO70769 IgG1 for binding to human activating receptors V158 and F158 FcγRIIIa, and the inhibitory receptor FcγRIIb, as measured by competition AlphaScreen assay. FIG. 80b shows the AlphaScreen data for select variants.

FIG. 81a-81b. Competition Surface Plasmon Resonance (SPR) experiment measuring binding affinities of I332E and S239D/I332E variants in the context of trastuzumab to human V158 FcγRIIIa. FIG. 81a provides the sensorgram raw data, FIG. 81b provides a plot of the log of receptor concentration against the initial rate obtained at each concentration, and FIG. 81c provides the affinities obtained from the fits to these data as described in Example 1.

FIG. 82. Cell-based ADCC assays of select Fc variants in the context of the anti-CD20 antibody PRO70769. ADCC was measured by the release of lactose dehydrogenase using a LDH Cytotoxicity Detection Kit (Roche Diagnostic). CD20+ lymphoma WIL2-S cells were used as target cells and human PBMCs were used as effector cells. Shown is the dose-dependence of ADCC on antibody concentration for the indicated antibodies, normalized to the minimum and maximum fluorescence signal for each particular curve, provided by the baselines at low and high antibody concentrations respectively. The curves represent the fits of the data to a sigmoidal dose-response model using nonlinear regression.

FIG. 83. Cell-based ADCC assay of select Fc variants in the context of PRO70769 IgG1 in the absence and presence of serum levels of human IgG. ADCC was measured by the release of lactose dehydrogenase using a LDH Cytotoxicity Detection Kit (Roche Diagnostic). CD20+ lymphoma WIL2-S cells were used as target cells and human PBMCs were used as effector cells.

FIG. 84. Residues mutated in Fc variants designed to enhance CDC. The structure of the human IgG1 Fc region is shown (pdb accession code 1E4K, Sondermann et al., 2000, Nature 406:267-273, hereby entirely incorporated by reference). Black ball and sticks indicate residues D270, K322, P329, and P331, which have been shown to be important in mediating binding to complement protein C1q, and grey sticks indicate residues that were mutated in the present invention to explore variants that affect CDC.

FIG. 85a-85b. Fc variants screened for complement-mediated cytotoxicity (CDC) and CDC data. The variable region is that of the anti-CD20 antibody PRO70769, and the heavy chain constant region is IgG1 unless noted IgG(1/2) ELLGG. Fold CDC provides the relative CDC activity compared to WT PRO70769 IgG1.

FIG. 86. CDC assays of Fc variant anti-CD20 antibodies. The dose-dependence on antibody concentration of complement-mediated lysis is shown for the indicated PRO70769 antibodies against CD20+WIL2-S lymphoma target cells. Lysis was measured using release of Alamar Blue, and data were normalized to the minimum and maximum fluorescence signal for each particular curve, provided by the baselines at low and high antibody concentrations respectively. The curves represent the fits of the data to a sigmoidal dose-response model with variable slope using nonlinear regression.

FIG. 87. Amino acid modifications that provide enhanced and reduced CDC, and positions that may be modified that may provide enhanced/modulated CDC. Positions are numbered according to the EU index.

FIG. 88. Fc variants screened for reduced FcγR affinity, FcγR-mediated effector function, and complement-mediated effector function. The variable region is that of the anti-CD20 antibody PRO70769, and the heavy chain constant region is IgG1. The figure provides the Fold IC50 for binding to human V158 FcγRIIIa and the Fold EC50 of CDC activity relative to WT PRO70769 IgG1.

FIG. 89. Binding to human V158 FcγRIIIa by select PRO70769 Fc variants as determined by the competition AlphaScreen assay.

FIG. 90. CDC assays of select Fc variant anti-CD20 antibodies against CD20+WIL2-S lymphoma target cells. Lysis was measured by Alamar Blue release.

FIG. 91. Cell-based ADCC activity of select anti-CD20 Fc variants against CD20+ lymphoma WIL2-S cells. Human PBMCs were used as effector cells, and lysis was measured by LDH release.

FIG. 92. Fc variants screened for reduced FcγR affinity, FcγR-mediated effector function, and complement-mediated effector function. The variable region is that of the anti-CD20 antibody PRO70769, and the heavy chain constant region is IgG1. The figure provides the Fold IC50 relative to WT for binding to human V158 FcγRIIIa by two separate experiments, the Fold IC50 relative to WT for binding to human FcγRI, and the Fold EC50 relative to WT for CDC activity.

FIG. 93. Binding to the low affinity human activating receptor V158 FcγRIIIa and the high affinity human activating receptor FcγRI by select PRO70769 Fc variants as determined by the competition AlphaScreen assay.

FIG. 94. CDC activity of select PRO70769 Fc variants against CD20+WIL2-S lymphoma target cells. Lysis was measured by release of Alamar Blue.

FIG. 95a-95b. Cell-based ADCC activity of anti-Her2 Fc variant and WT IgG antibodies against Her2/neu+SkBr-3 breast carcinoma target cells. Human PBMCs were used as effector cells, and lysis was measured by LDH release.

FIG. 96a-96d. Sequences showing possible constant heavy chain sequences with reduced Fc ligand binding and effector function properties (FIG. 96a), and sequences of improved anti-CTLA-4 antibodies (FIGS. 96b-96d). FIG. 96a shows potential Fc variant constant heavy chain sequences, with variable positions designated in bold as X1, X2, X3, X4, X5, X6, X7, and X8. The table below the sequence provides the WT amino acid and possible substitutions for these positions. Improved antibody sequences may comprise one or more non-WT amino acid selected from this group of possible modifications. FIG. 96b provides the light chain sequence of an anti-CTLA-4 antibody, and FIGS. 96c and 96d provide heavy chain sequences of anti-CTLA-4 antibodies with reduced Fc ligand binding and Fc-mediated effector function. These include an L235G/G236R IgG1 heavy chain (FIG. 96c) and an IgG2 heavy chain (FIG. 96d). The positions are numbered according to the EU index as in Kabat, and thus do not correspond to the sequential order in the sequence.

FIG. 97. Residues at which amino acid modifications were made in the Fc variants of the present invention, mapped onto the Fc/FcγRIIIb complex structure 1IIS. Fc is shown as a gray ribbon diagram, and FcγRIIIb is shown as a black ribbon. Experimental library residues are shown in black, the N297 carbohydrate is shown in grey.

FIG. 98. Expression of Fc variant and wild type (WT) proteins of alemtuzumab in 293T cells. Plasmids containing alemtuzumab heavy chain genes (WT or variants) were co-transfected with plasmid containing the alemtuzumab light chain gene. Media were harvested 5 days after transfection. For each transfected sample, 10 ul medium was loaded on a SDS-PAGE gel for Western analysis. The probe for Western was peroxidase-conjugated goat-anti human IgG (Jackson Immuno-Research, catalog #109-035-088). WT: wild type alemtuzumab; 1-10: alemtuzumab variants. H and L indicate antibody heavy chain and light chain, respectively.

FIG. 99. Purification of alemtuzumab using protein A chromatography. WT alemtuzumab proteins was expressed in 293T cells and the media was harvested 5 days after transfection. The media were diluted 1:1 with PBS and purified with protein A (Pierce, Catalog #20334). 0: original sample before purification; FT: flow through; E: elution; C: concentrated final sample. The left picture shows a Simple Blue-stained SDS-PAGE gel, and the right shows a western blot labeled using peroxidase-conjugated goat-anti human IgG.

FIG. 100. Production of deglycosylated antibodies. Wild type and variants of alemtuzumab were expressed in 293T cells and purified with protein A chromatography. Antibodies were incubated with peptide-N-glycosidase (PNGase F) at 37° C. for 24h. For each antibody, a mock treated sample (−PNGase F) was done in parallel. WT: wild-type alemtuzumab; #15, #16, #17, #18, #22: alemtuzumab variants F241E/F243R/V262E/V264R, F241E/F243Q/V262T/V264E, F241R/F243Q/V262T/V264R, F241E/F243Y/V262T/V264R, and I332E respectively. The faster migration of the PNGase F treated versus the mock treated samples represents the deglycosylated heavy chains.

FIG. 101. Alemtuzumab expressed from 293T cells binds its antigen. The antigenic CD52 peptide, fused to GST, was expressed in E. coli BL21 (DE3) under IPTG induction. Both uninduced and induced samples were run on a SDS-PAGE gel, and transferred to PVDF membrane. For western analysis, either alemtuzumab from Sotec (a-CD52, Sotec) (final concentration 2.5 ng/ul) or media of transfected 293T cells (Campath, Xencor) (final alemtuzumab concentration approximately 0.1-0.2 ng/ul) were used as primary antibody, and peroxidase-conjugated goat-anti human IgG was used as secondary antibody. M: pre-stained marker; U: un-induced sample for GST-CD52; I: induced sample for GST-CD52.

FIG. 102. Expression and purification of extracellular region of human V158 FcγRIIIa. Tagged FcγRIIIa was transfected in 293T cells, and media containing secreted FcγRIIIa were harvested 3 days later and purified using affinity chromatography. 1: media; 2: flow through; 3: wash; 4-8: serial elutions. Both simple blue-stained SDS-PAGE gel and western result are shown. For the western blot, membrane was probed with anti-GST antibody.

FIG. 103. Binding to human V158 FcγRIIIa by select alemtuzumab Fc variants from the experimental library as determined by the AlphaScreen™ assay, described in Example 2. In the presence of competitor antibody (Fc variant or WT alemtuzumab) a characteristic inhibition curve is observed as a decrease in luminescence signal. Phosphate buffer saline (PBS) alone was used as the negative control. The binding data were normalized to the maximum and minimum luminescence signal for each particular curve, provided by the baselines at low and high antibody concentrations respectively. The curves represent the fits of the data to a one site competition model using nonlinear regression. These fits provide IC50s for each antibody, illustrated for WT and S239D by the dotted lines.

FIGS. 104a and 104b. AlphaScreen assay showing binding of select alemtuzumab (FIG. 104a) and trastuzumab (FIG. 104b) Fc variants to human Val158 FcγRIIIa. The binding data were normalized to the upper and lower baselines for each particular antibody, and the curves represent the fits of the data to a one site competition model. PBS was used as a negative control.

FIG. 105. AlphaScreen assay showing binding of select alemtuzumab Fc variants to human FcγRIIb. The binding data were normalized to the upper and lower baselines for each particular antibody, and the curves represent the fits of the data to a one site competition model. PBS was used as a negative control.

FIG. 106. AlphaScreen assay showing binding of select alemtuzumab Fc variants to human R131 FcγRIIa. The binding data were normalized to the upper and lower baselines for each particular antibody, and the curves represent the fits of the data to a one site competition model.

FIG. 107. AlphaScreen assay measuring binding of select alemtuzumab Fc variants to human FcRn, as described in Example 2. The binding data were normalized to the upper and lower baselines for each particular antibody, and the curves represent the fits of the data to a one site competition model. PBS was used as a negative control.

FIG. 108. AlphaScreen assay measuring binding of select alemtuzumab Fc variants to bacterial protein A, as described in Example 2. The binding data were normalized to the upper and lower baselines for each particular antibody, and the curves represent the fits of the data to a one site competition model. PBS was used as a negative control.

FIGS. 109a-109b. AlphaScreen assay comparing binding of select alemtuzumab Fc variants to human V158 FcγRIIIa (FIG. 109a) and human FcγRIIb (FIG. 109b). The binding data were normalized to the upper and lower baselines for each particular antibody, and the curves represent the fits of the data to a one site competition model. PBS was used as a negative control.

FIGS. 110a-110c. AlphaScreen assay measuring binding to human V158 FcγRIIIa (FIGS. 110a and 110b) and human FcγRIIb (FIG. 110c) by select Fc variants in the context of trastuzumab. The binding data were normalized to the upper and lower baselines for each particular antibody, and the curves represent the fits of the data to a one site competition model. PBS was used as a negative control.

FIG. 111. AlphaScreen assay measuring binding to human V158 FcγRIIIa by select Fc variants in the context of rituximab. The binding data were normalized to the upper and lower baselines for each particular antibody, and the curves represent the fits of the data to a one site competition model. PBS was used as a negative control.

FIG. 112. AlphaScreen assay measuring binding to human V158 FcγRIIIa by select Fc variants in the context of cetuximab. The binding data were normalized to the upper and lower baselines for each particular antibody, and the curves represent the fits of the data to a one site competition model. PBS was used as a negative control.

FIGS. 113a-113b. AlphaScreen assay showing binding of select alemtuzumab Fc variants to the V158 (FIG. 113a) and F158 (FIG. 113b) allotypes of human FcγRIIIa. The binding data were normalized to the upper and lower baselines for each particular antibody, and the curves represent the fits of the data to a one site competition model. PBS was used as a negative control.

FIGS. 114a-114d. FIGS. 114a and 114b show the correlation between SPR Kd's and AlphaScreen IC50's from binding of select alemtuzumab Fc variants to V158 FcγRIIIa (FIG. 21a) and F158 FcγRIIIa (FIG. 114b). FIGS. 114c and 114d show the correlation between SPR and AlphaScreen fold-improvements over WT for binding of select alemtuzumab Fc variants to V158 FcγRIIIa (FIG. 114c) and F158 FcγRIIIa (FIG. 114d). Binding data are presented in Table 3. The lines through the data represent the linear fits of the data, and the r2 values indicate the significance of these fits.

FIGS. 115a and 115b. AlphaScreen assay showing binding of select alemtuzumab Fc variants to human V158 FcγRIIIa. The binding data were normalized to the upper and lower baselines for each particular antibody, and the curves represent the fits of the data to a one site competition model. PBS was used as a negative control.

FIGS. 116a-116b. Cell-based ADCC assays of select Fc variants in the context of alemtuzumab. ADCC was measured using the DELFIA® EuTDA-based cytotoxicity assay (Perkin Elmer, MA), as described in Example 3, using DoHH-2 lymphoma target cells and 50-fold excess human PBMCs. FIG. 116a is a bar graph showing the raw fluorescence data for the indicated alemtuzumab antibodies at 10 ng/ml. The PBMC bar indicates basal levels of cytotoxicity in the absence of antibody. FIG. 116b shows the dose-dependence of ADCC on antibody concentration for the indicated alemtuzumab antibodies, normalized to the minimum and maximum fluorescence signal for each particular curve, provided by the baselines at low and high antibody concentrations respectively. The curves represent the fits of the data to a sigmoidal dose-response model using nonlinear regression.

FIGS. 117a-117c. Cell-based ADCC assays of select Fc variants in the context of trastuzumab. ADCC was measured using the DELFIA® EuTDA-based cytotoxicity assay, as described in Example 3, using BT474 and Sk-Br-3 breast carcinoma target cells and 50-fold excess human PBMCs. FIG. 117a is a bar graph showing the raw fluorescence data for the indicated trastuzumab antibodies at 1 ng/ml. The PBMC bar indicates basal levels of cytotoxicity in the absence of antibody. FIGS. 117b and 117c show the dose-dependence of ADCC on antibody concentration for the indicated trastuzumab antibodies, normalized to the minimum and maximum fluorescence signal for each particular curve, provided by the baselines at low and high antibody concentrations respectively. The curves represent the fits of the data to a sigmoidal dose-response model using nonlinear regression.

FIGS. 118a-118c. Cell-based ADCC assays of select Fc variants in the context of rituximab. ADCC was measured using the DELFIA® EuTDA-based cytotoxicity assay, as described in Example 3, using WIL2-S lymphoma target cells and 50-fold excess human PBMCs. FIG. 118a is a bar graph showing the raw fluorescence data for the indicated rituximab antibodies at 1 ng/ml. The PBMC bar indicates basal levels of cytotoxicity in the absence of antibody. FIGS. 118b and 118c show the dose-dependence of ADCC on antibody concentration for the indicated rituximab antibodies, normalized to the minimum and maximum fluorescence signal for each particular curve, provided by the baselines at low and high antibody concentrations respectively. The curves represent the fits of the data to a sigmoidal dose-response model using nonlinear regression.

FIGS. 119a-119c. Cell-based ADCC assay of select trastuzumab (FIG. 119a), rituximab (FIG. 119b), and PRO70769 (FIG. 119c) Fc variants showing enhancements in potency and efficacy. Both assays used homozygous F158/F158 FcγRIIIa PBMCs as effector cells at a 25-fold excess to target cells, which were Sk-Br-3 for the trastuzumab assay and WIL2-S for the rituximab assay. Data were normalized according to the absolute minimal lysis for the assay, provided by the fluorescence signal of target cells in the presence of PBMCs alone (no antibody), and the absolute maximal lysis for the assay, provided by the fluorescence signal of target cells in the presence of Triton X1000, as described in Example 3.

FIG. 120. AlphaScreen assay showing binding of select alemtuzumab Fc variants to human V158 FcγRIIIa. The binding data were normalized to the upper and lower baselines for each particular antibody, and the curves represent the fits of the data to a one site competition model. PBS was used as a negative control.

FIG. 121. ADCC. Cell-based ADCC assays of select Fc variant trastuzumab antibodies as compared to WT trastuzumab. Purified human peripheral blood monocytes (PBMCs) were used as effector cells, and Sk-Br-3 breast carcinoma cells were used as target cells. Lysis was monitored by measuring LDH activity using the Cytotoxicity Detection Kit (LDH, Roche Diagnostic Corporation, Indianapolis, Ind.). Samples were run in triplicate to provide error estimates (n=3, +/−S.D.). The figure shows the dose dependence of ADCC at various antibody concentrations, normalized to the minimum and maximum levels of lysis for the assay. The curves represent the fits of the data to a sigmoidal dose-response model using nonlinear regression.

FIGS. 122a-122b. Cell-based ADCC assay of select trastuzumab Fc variants against different cell lines expressing varying levels of the Her2/neu target antigen. ADCC assays were run as described in Example 5, with various cell lines expressing amplified to low levels of Her2/neu receptor, including Sk-Br-3 (1×106 copies), SkOV3 (˜1×105), OVCAR3 (˜1×104), and MCF-7 (˜3×103 copies). FIG. 122a provides a western blot showing the Her2 expression level for each cell line; equivalent amounts of cell lysate were loaded on an SDS-PAGE gel, and Her2 was detected using trastuzumab. Human PBMCs allotyped as homozygous F158/F158 FcγRIIIa were used at 25-fold excess to target cells. The bar graph in FIG. 122b provides ADCC data for WT and Fc variant against the indicated cell lines, normalized to the minimum and maximum fluorescence signal provided by minimal lysis (PBMCs alone) and maximal lysis (Triton X1000).

FIG. 123. Cell-based ADCC assays of select Fc variants in the context of trastuzumab using natural killer (NK) cells as effector cells and measuring LDH release to monitor cell lysis. NK cells, allotyped as heterozygous F158/F158 FcγRIIIa, were at an 4-fold excess to Sk-Br-3 breast carcinoma target cells, and the level of cytotoxicity was measured using the LDH Cytotoxicity Detection Kit, according to the manufacturer's protocol (Roche Diagnostics GmbH, Penzberg, Germany). The graph shows the dose-dependence of ADCC on antibody concentration for the indicated trastuzumab antibodies, normalized to the minimum and maximum fluorescence signal for each particular curve, provided by the baselines at low and high antibody concentrations respectively. The curves represent the fits of the data to a sigmoidal dose-response model using nonlinear regression.

FIG. 124a-124b. Cell-based ADCP assay of select variants. The ADCP assay was carried out as described in Example 7, using a co-labeling strategy coupled with flow cytometry. Differentiated macrophages were used as effector cells, and Sk-Br-3 cells were used as target cells. FIG. 124a: percent phagocytosis represents the number of co-labeled cells (macrophage+Sk-Br-3) over the total number of Sk-Br-3 in the population (phagocytosed+non-phagocytosed). FIG. 124b shows a cell-based ADCP enhancement of variant rituximab antibodies against WIL2-S target cells. % ADCP represents the number of cells co-labeled with PKH76 and PKH26 (macrophage+target) over the total number of target cells in the population (phagocytosed+non-phagocytosed) after 10,000 counts.

FIGS. 125a-125c. Capacity of select Fc variants to mediate binding and activation of complement. FIG. 125a shows an AlphaScreen assay measuring binding of select alemtuzumab Fc variants to C1q. The binding data were normalized to the upper and lower baselines for each particular antibody, and the curves represent the fits of the data to a one site competition model. FIGS. 125b and 125c show a cell-based assay measuring capacity of select rituximab Fc variants to mediate CDC. CDC assays were performed using Alamar Blue to monitor lysis of Fc variant and WT rituximab—opsonized WIL2-S lymphoma cells by human serum complement (Quidel, San Diego, Calif.). The dose-dependence on antibody concentration of complement-mediated lysis is shown for the indicated rituximab antibodies, normalized to the minimum and maximum fluorescence signal for each particular curve, provided by the baselines at low and high antibody concentrations respectively. The curves represent the fits of the data to a sigmoidal dose-response model using nonlinear regression.

FIGS. 126a-126e. Enhanced B cell depletion by Fc variants in macaques, as described in Example 9. FIG. 126a shows the percent B cells remaining in Macaca Fascicularis monkeys during treatment with anti-CD20 WT and S239D/I332E rituximab antibodies, measured using markers CD20+ and CD40+. FIG. 126b shows the percent natural killer (NK) cells remaining in the monkeys during treatment, measured using markers CD3−/CD16+ and CD3−/CD8+. FIG. 126c shows the dose response of CD20+ B cell levels to treatment with S239D/I332E rituximab. Data are presented as the average of 3 monkeys/sample. FIG. 126d shows percent CD3−/CD8+NK cells remaining during treatment. FIG. 126e shows percent CD3−/CD16+NK cells remaining during treatment. n=3.

FIGS. 127a-127c. AlphaScreen assay measuring binding of select alemtuzumab (FIG. 127a) and trastuzumab (FIG. 127b) Fc variants to mouse FcγRIII, as described in Example 10. The binding data were normalized to the upper and lower baselines for each particular antibody, and the curves represent the fits of the data to a one site competition model. PBS was used as a negative control.

FIG. 128. Cell-based ADCC assays of select Fc variants in the context of trastuzumab using mouse PBMCs as effector cells. ADCC was measured using the DELFIA® EuTDA-based cytotoxicity assay using Sk-Br-3 breast carcinoma target cells and 8-fold excess mouse PBMCs. The bar graph shows the raw fluorescence data for the indicated trastuzumab antibodies at 10 ng/ml. The PBMC bar indicates basal levels of cytotoxicity in the absence of antibody, and TX indicates complete cell lysis in the presence of Triton X1000.

FIG. 129. AlphaScreen assay measuring binding to human V158 FcγRIIIa by select trastuzumab Fc variants expressed in 293T and CHO cells, as described in Example 11. The binding data were normalized to the upper and lower baselines for each particular antibody, and the curves represent the fits of the data to a one site competition model. PBS was used as a negative control.

FIGS. 130a-130b. Synergy of Fc variants and engineered glycoforms. FIG. 130a presents an AlphaScreen assay showing V158 FcγRIIIa binding by WT and Fc variant (V209, S239/I332E/A330L) trastuzumab expressed in 293T, CHO, and Lec-13 CHO cells. The data were normalized to the upper and lower baselines for each antibody, and the curves represent the fits of the data to a one site competition model. PBS was used as a negative control. FIG. 130b presents a cell-based ADCC assay showing the ability of 239T, CHO, and Lec-13 CHO expressed WT and V209 trastuzumab to mediate ADCC. ADCC was measured using the DELFIA® EuTDA-based cytotoxicity assay as described previously, with Sk-Br-3 breast carcinoma target cells. The data show the dose-dependence of ADCC on antibody concentration for the indicated trastuzumab antibodies, normalized to the minimum and maximum fluorescence signal for each particular curve, provided by the baselines at low and high antibody concentrations respectively. The curves represent the fits of the data to a sigmoidal dose-response model using nonlinear regression.

FIG. 131. AlphaScreen assay showing binding of aglycosylated alemtuzumab Fc variants to human V158 FcγRIIIa. The binding data were normalized to the upper and lower baselines for each particular antibody, and the curves represent the fits of the data to a one site competition model. PBS was used as a negative control.

FIG. 132. AlphaScreen assay comparing human V158 FcγRIIIa binding by select alemtuzumab Fc variants in glycosylated (solid symbols, solid lines) and deglycosylated (open symbols, dotted lines). The binding data were normalized to the upper and lower baselines for each particular antibody, and the curves represent the fits of the data to a one site competition model.

FIGS. 133a-133c. Sequences showing improved anti-CD20 antibodies. The light and heavy chain sequences of rituximab are presented in FIG. 133a and FIG. 133b respectively, and are taken from translated Sequence 3 of U.S. Pat. No. 5,736,137. Relevant positions in FIG. 133b are bolded, including S239, V240, V264I, H268, E272, K274, N297, S298, K326, A330, and 1332. FIG. 133c shows the improved anti-CD20 antibody heavy chain sequences, with variable positions designated in bold as X1, X2, X3, X4, X5, X6, X7, X8, X9, Z1, and Z2. The table below the sequence provides possible substitutions for these positions. The improved anti-CD20 antibody sequences comprise at least one non-WT amino acid selected from the group of possible substitutions for X1, X2, X3, X4, X5, X6, X7, X8, and X9. These improved anti-CD20 antibody sequences may also comprise a substitution Z1 and/or Z2. These positions are numbered according to the EU index as in Kabat, and thus do not correspond to the sequential order in the sequence.

FIG. 134 depicts an amino acid sequence of the invention, wherein the particular Xaa residues are as shown in Table 10.

FIG. 135. AlphaScreen assay measuring binding to human V158 FcγRIIIa by select Fc variants in the context of PRO70769. The binding data were normalized to the upper and lower baselines for each particular antibody, and the curves represent the fits of the data to a one site competition model. PBS was used as a negative control.

FIGS. 136a-136c provide the heavy chain sequence (FIG. 136a), light chain sequence (FIG. 136b), antigen specificity, and various indications (FIG. 136c) for sixteen select Fc variants (designated by numbers >1 through >16) which have demonstrated function in animal and/or clinical studies. Residues of the CDR1, CDR2, and CDR3 in the heavy chain for each of the sixteen exemplary variants are found within the region designated as VH CDR1, VH CDR2, VH CDR3, respectively, in FIG. 136a. Residues forming CDR1, CDR2, CDR3 in the light chain for each of the sixteen exemplary variants are found within the region designated as VL CDR1, VL CDR2, VL CDR3, respectively, in FIG. 136b. The EU index is provided in the uppermost row of FIGS. 136a-b as a frame of reference for the antibody sequences.

FIG. 137 provides a matrix representing a repertoire of possible Fc variants with different antigen specificities into an IgG scaffold selected from IgG1, IgG2, IgG3, IgG4, and IgG1/IgG2.

FIGS. 138a and 138b. AlphaScreen™ assay measuring binding to human V158 FcγRIIIa by select Fc variants in the context of trastuzumab. The binding data were normalized to the upper and lower baselines for each particular antibody, and the curves represent the fits of the data to a one site competition model. PBS was used as a negative control.

FIGS. 139a and 139b. AlphaScreen™ assay measuring binding to human V158 FcγRIIIa by select Fc variants in the context of rituximab (FIG. 139a) and cetuximab (FIG. 139b). The binding data were normalized to the upper and lower baselines for each particular antibody, and the curves represent the fits of the data to a one site competition model. PBS was used as a negative control.

FIG. 140. AlphaScreen™ assay showing binding of select alemtuzumab Fc variants to human R131 FcγRIIa. The binding data were normalized to the upper and lower baselines for each particular antibody, and the curves represent the fits of the data to a one site competition model.

FIG. 141a-141b. AlphaScreen™ assay showing binding of select alemtuzumab Fc variants to human V158 FcγRIIIa. The binding data were normalized to the upper and lower baselines for each particular antibody, and the curves represent the fits of the data to a one site competition model. PBS was used as a negative control.

FIG. 142. AlphaScreen™ assay measuring binding of select alemtuzumab Fc variants to bacterial protein A, as described in Example 10. The binding data were normalized to the upper and lower baselines for each particular antibody, and the curves represent the fits of the data to a one site competition model. PBS was used as a negative control.

FIG. 143. AlphaScreen™ assay measuring binding of select alemtuzumab Fc variants to human FcRn, as described in Example 10. The binding data were normalized to the upper and lower baselines for each particular antibody, and the curves represent the fits of the data to a one site competition model. PBS was used as a negative control.

### DETAILED DESCRIPTION OF THE INVENTION

### I. Overview

In addition, each modification discussed herein can be done independently or in combination with any other modification(s), and can be done on the Fc region of any or all of an IgG1, an IgG2, an IgG3, an IgG4, and an IgG1/IgG2 hybrid scaffold.

The present invention is directed to proteins comprising altered Fc regions that exhibit altered functionality, including differential binding to one or more Fcγ receptors as compared to a non-altered Fc region. In a particular embodiment, the variants reduce functionality, leading to desirable biological properties. The variants can include one or more insertions of an amino acid, one or more deletions, and/or one or more amino acid substitutions, as outlined herein.

The present invention provides engineering methods that may be used to generate Fc variants with optimized properties. A principal obstacle that has hindered previous attempts at Fc engineering is that only random attempts at modification have been possible, due in part to the inefficiency of engineering strategies and methods, and to the low-throughput nature of antibody production and screening. The present invention describes engineering methods that overcome these shortcomings. A variety of design strategies, computational screening methods, library generation methods, and experimental production and screening methods are contemplated. These strategies, approaches, techniques, and methods may be applied individually or in various combinations to engineer optimized anti-CD20 antibodies. Design strategies and computational screening methods are disclosed, for example, in U.S. Ser. No. 10/822,231, U.S. Ser. No. 10/672,280, and U.S. Ser. No. 10/379,392, incorporated by reference herein.

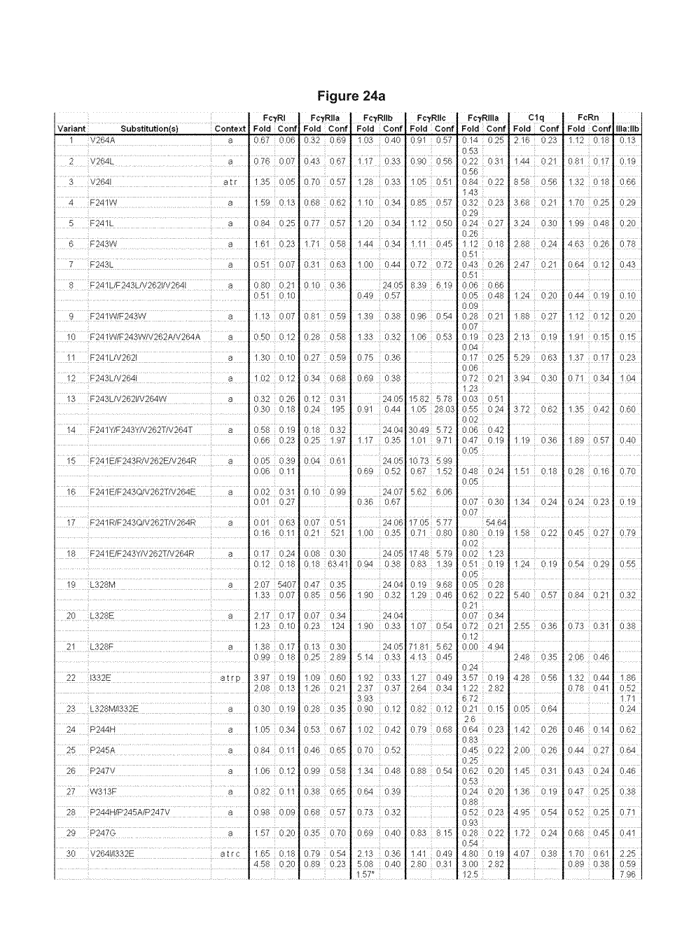

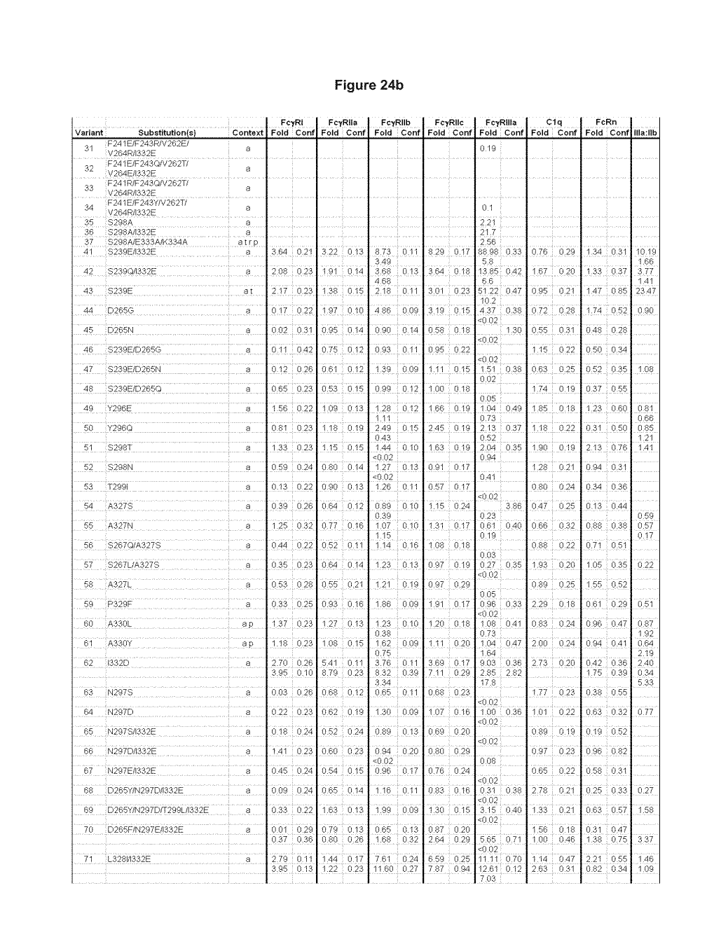

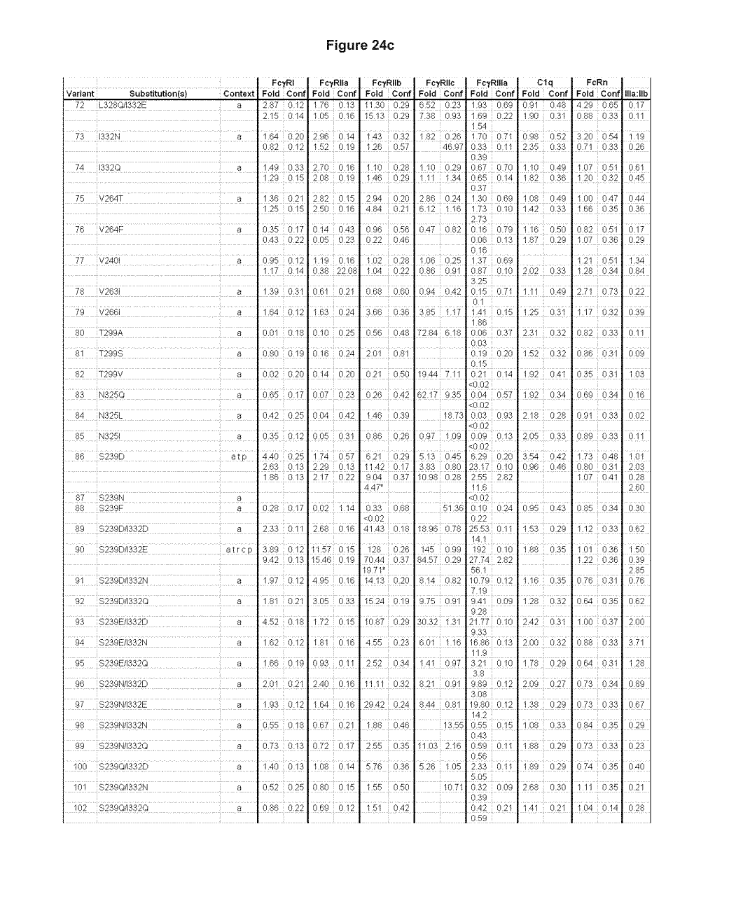

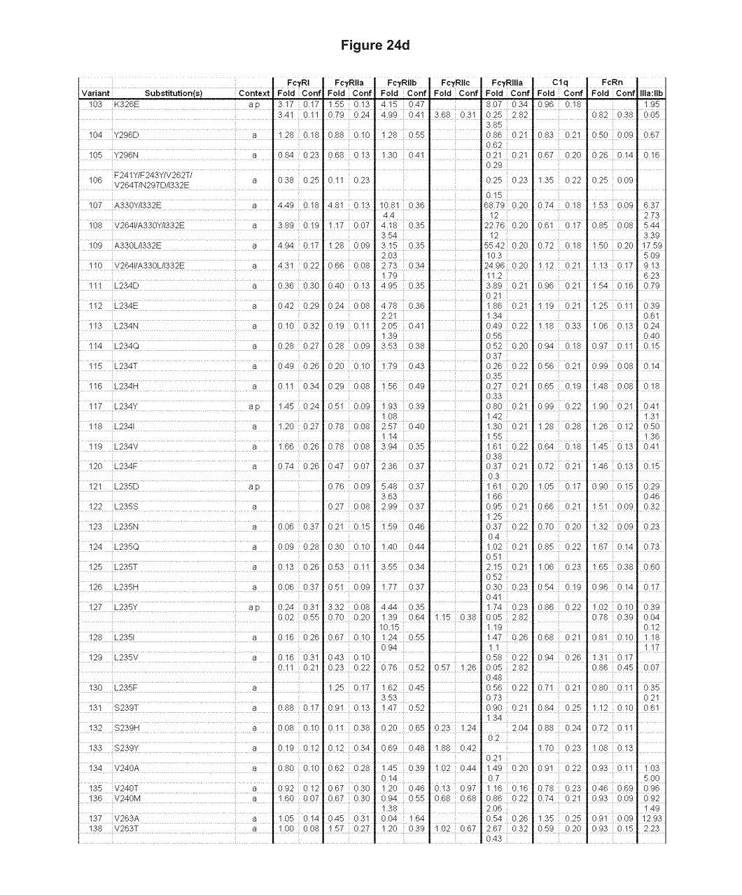

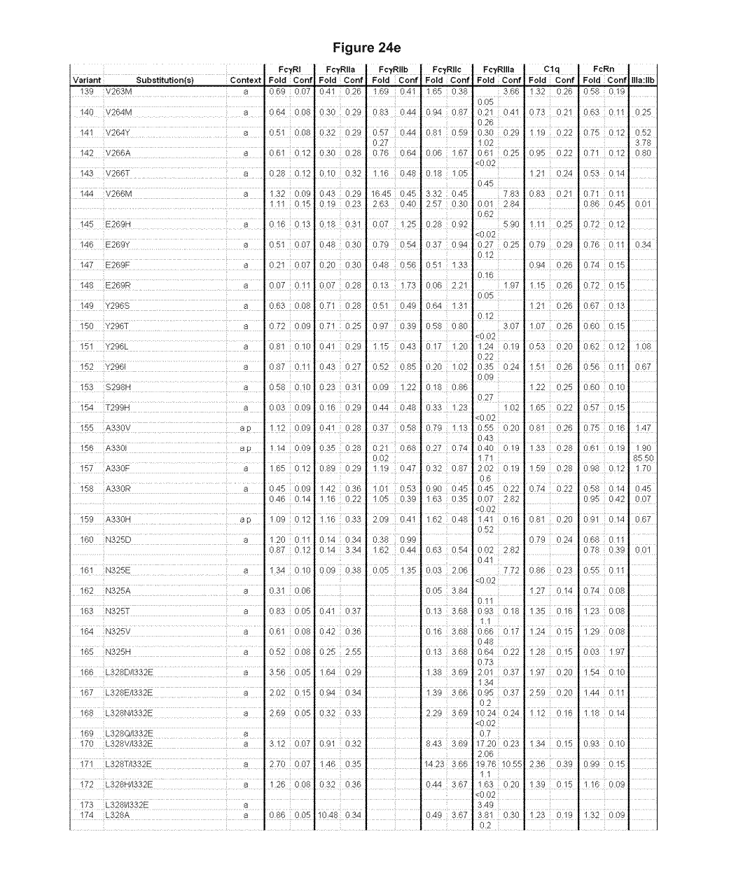

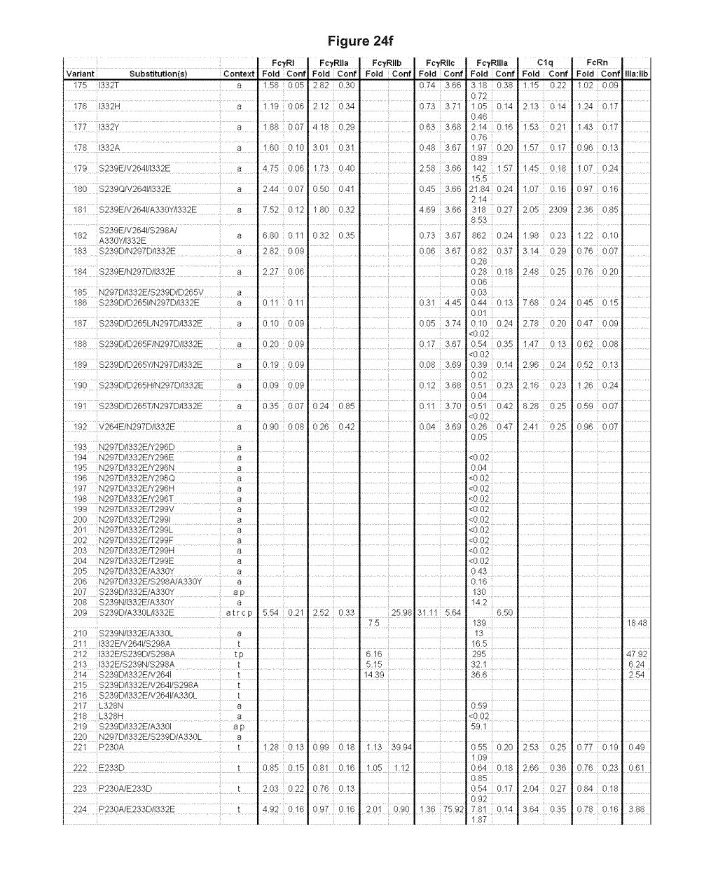

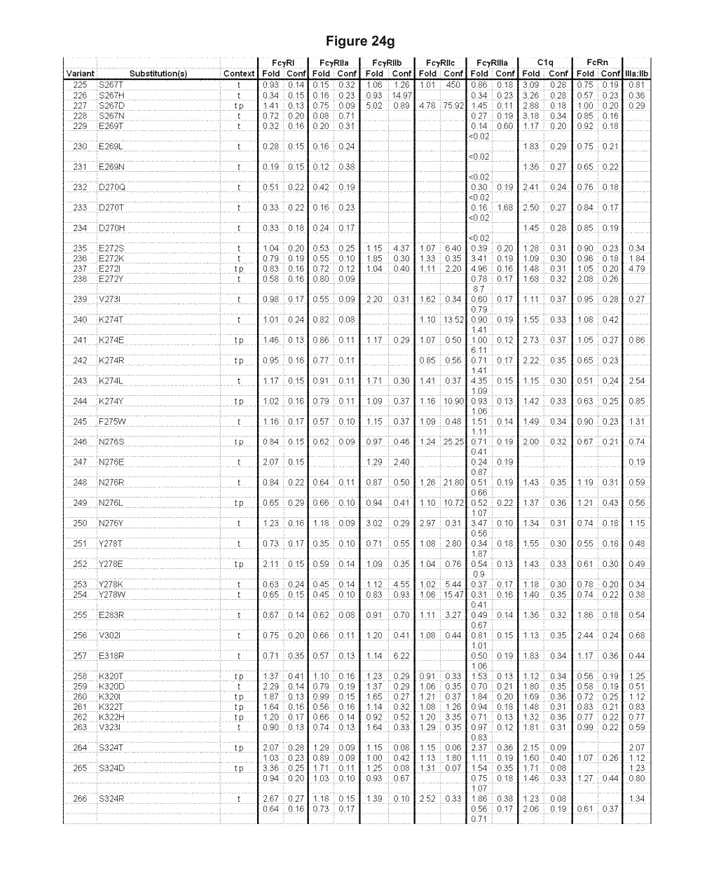

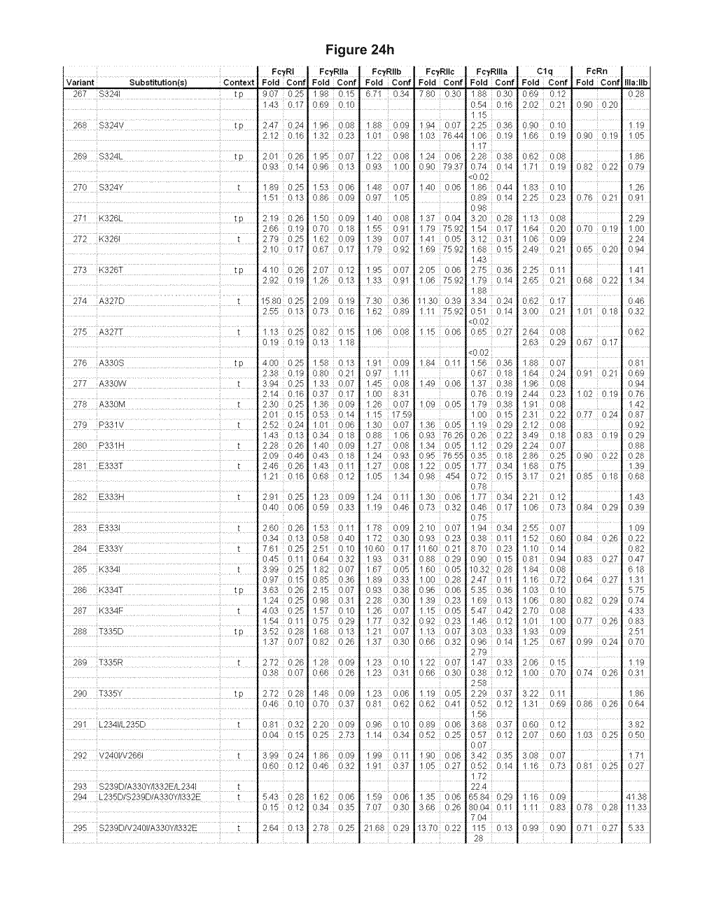

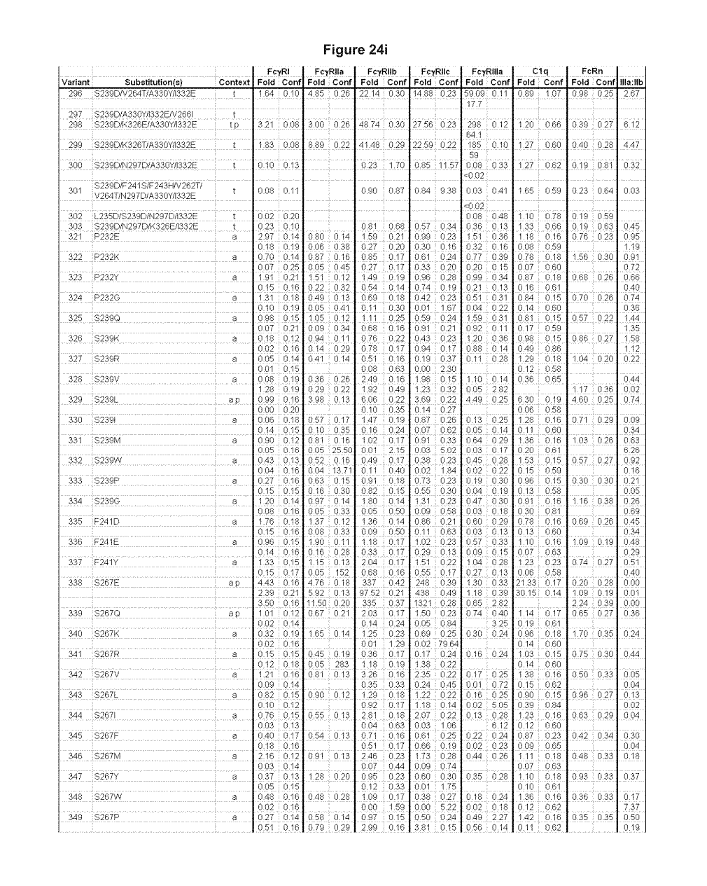

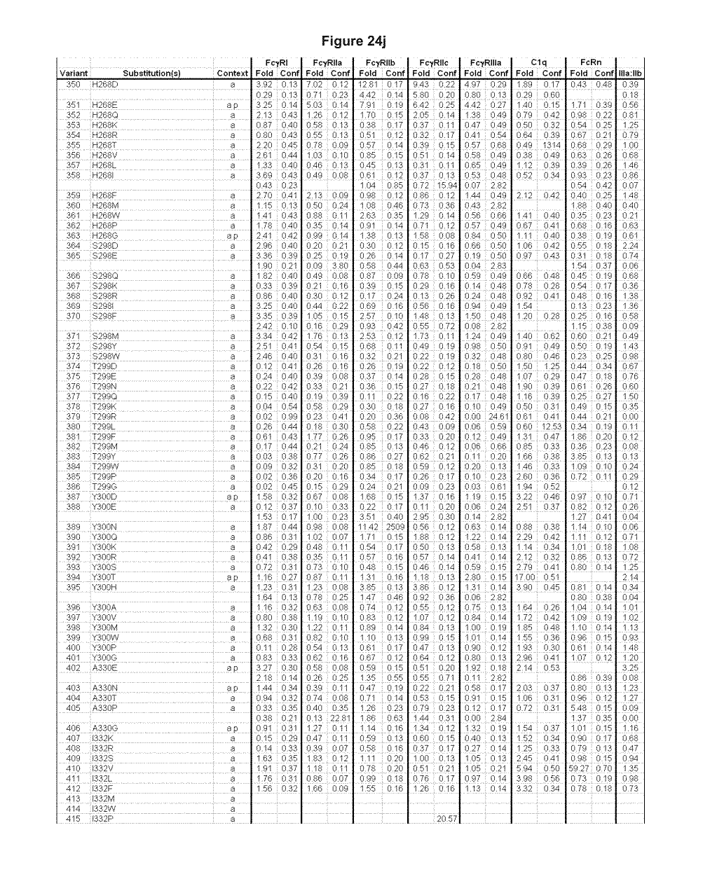

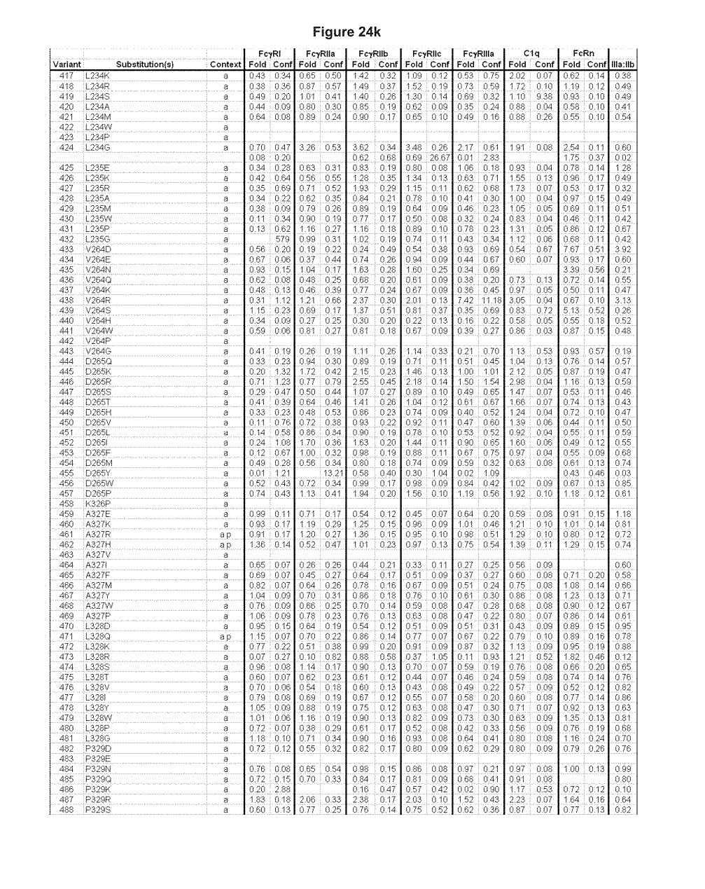

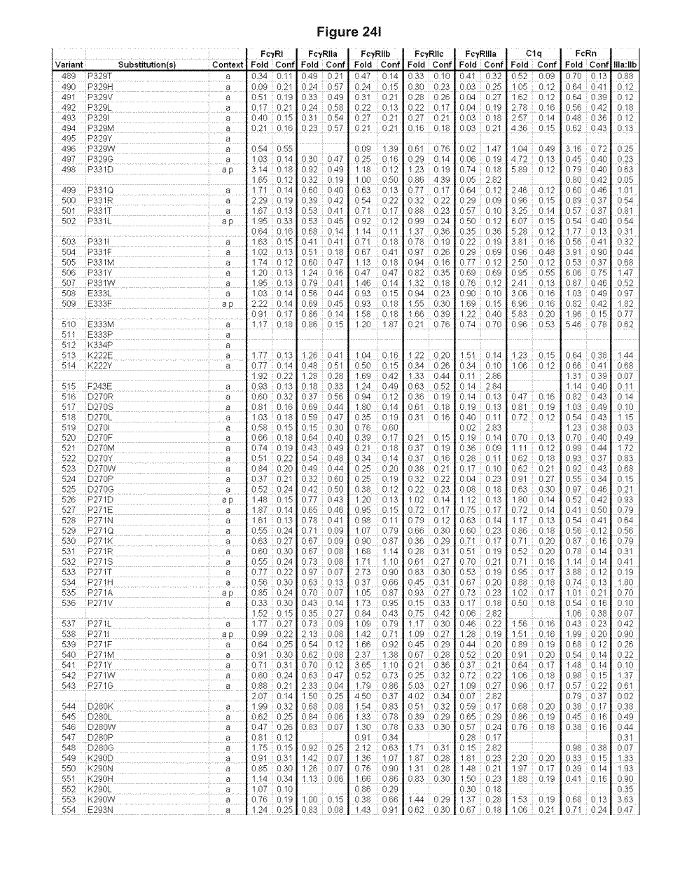

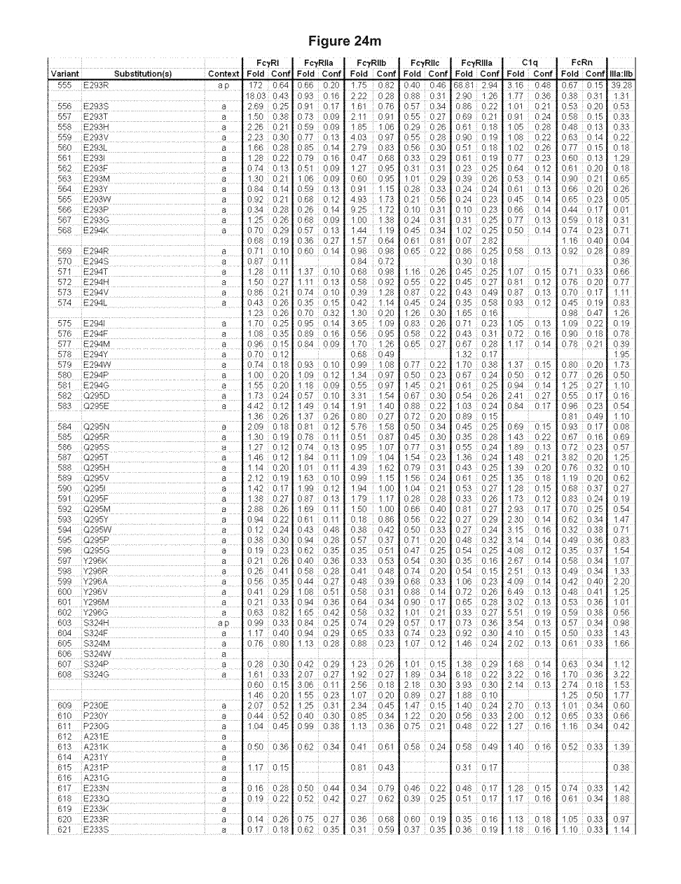

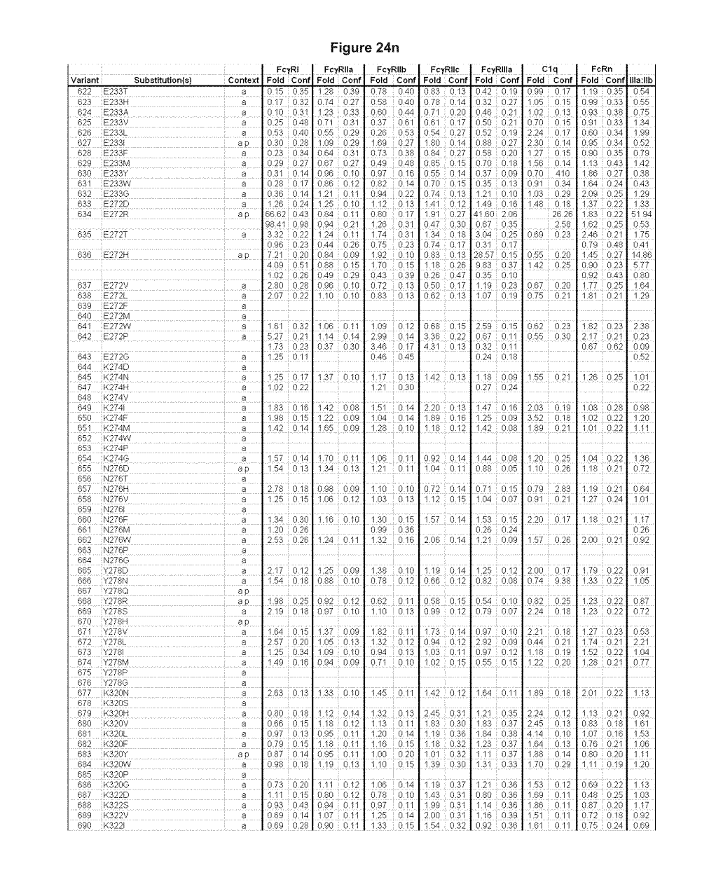

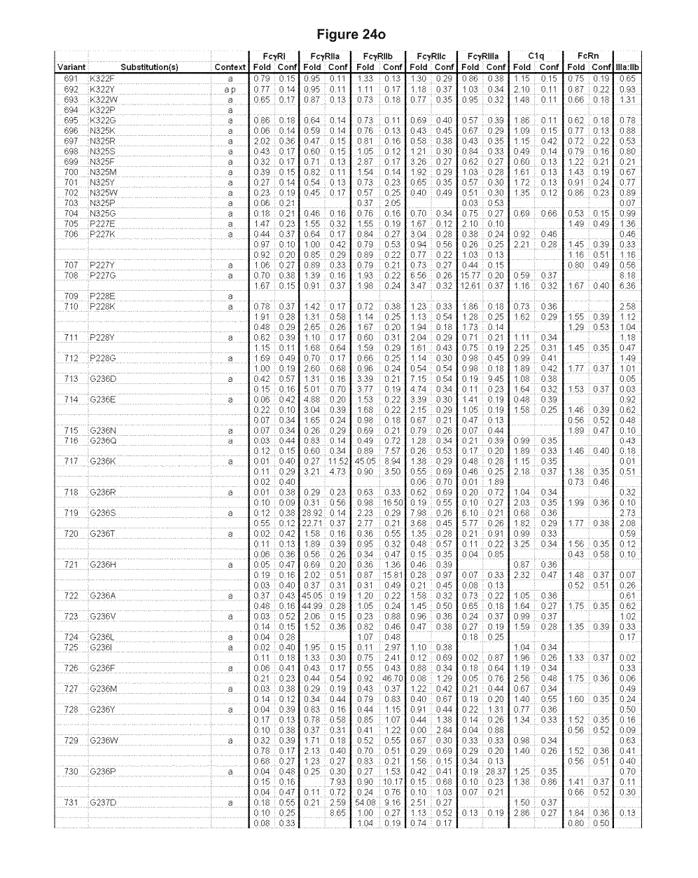

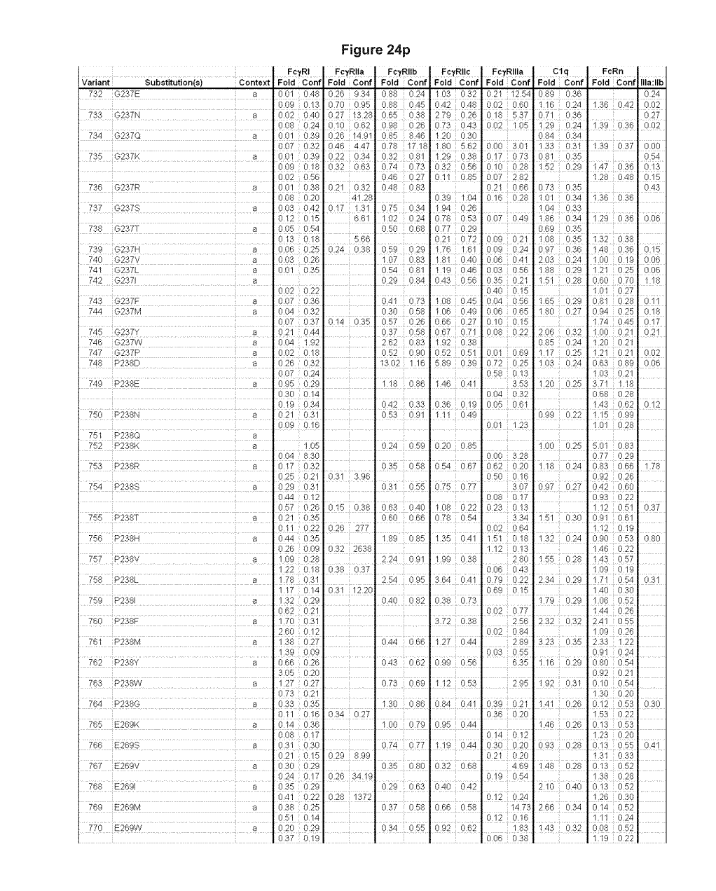

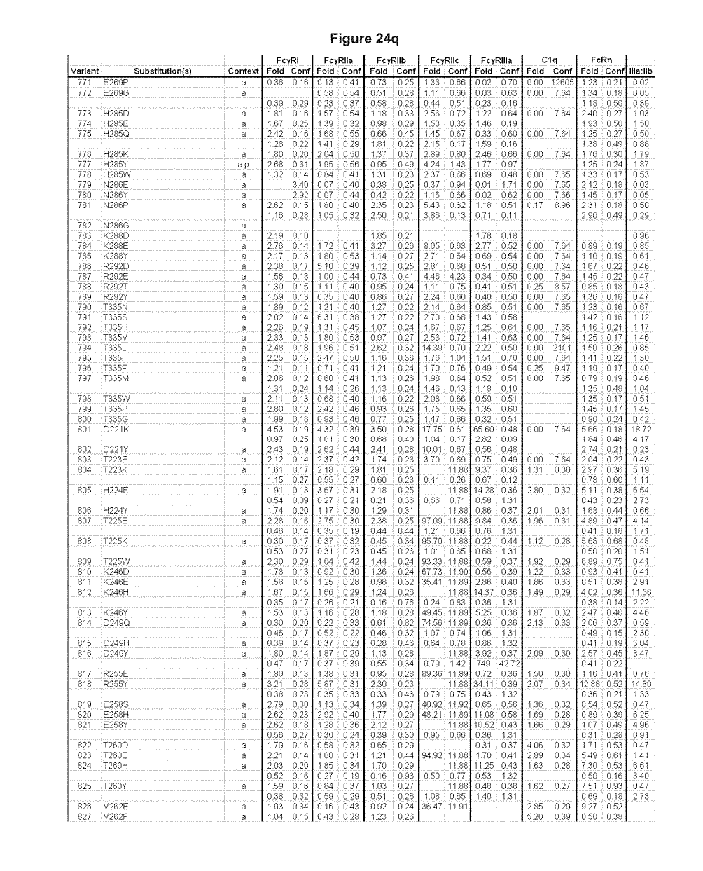

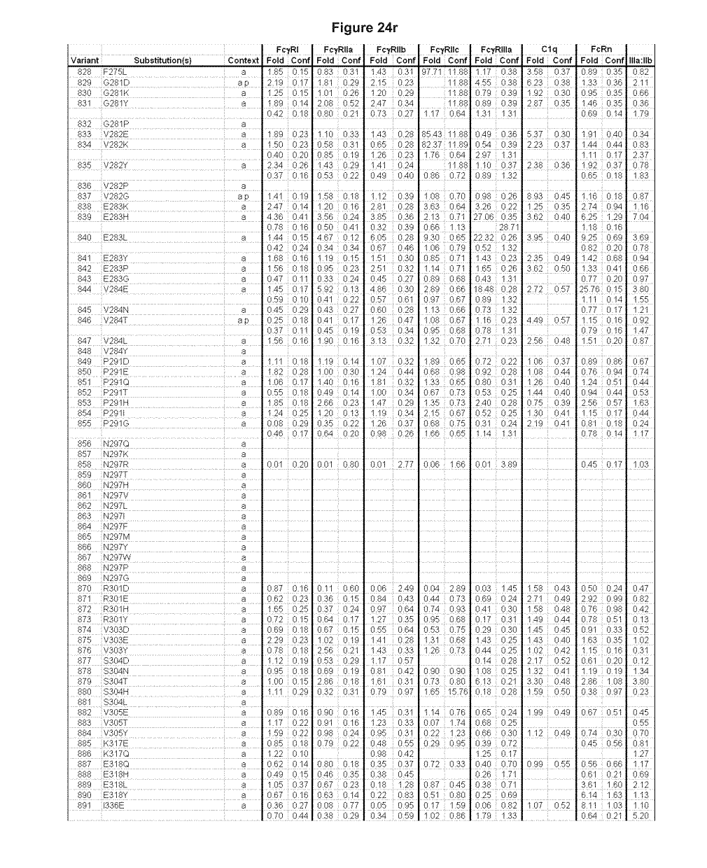

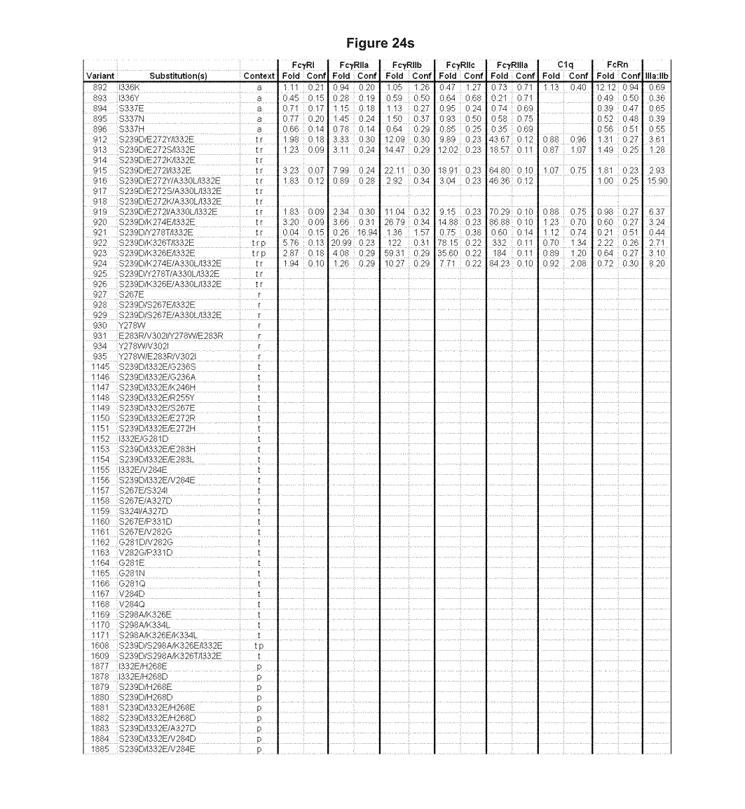

Specifically, amino acid variations as shown in FIG. 24 and outlined in U.S. Ser. Nos. 10/672,280, 10/822,231, 11/124,620, 11/174,287, 11/396,495; 11/538,406, 11/538,411 and 60/886,635 include a variety of disclosures, all of which are expressly incorporated by reference herein, and in particular for the disclosure of positions, particular substitutions, data and the figures.

### II. Definitions

In order that the application may be more completely understood, several definitions are set forth below. Such definitions are meant to encompass grammatical equivalents.

By “ablation” herein is meant a decrease or removal of activity. Thus for example, “ablating FcγR binding” means the Fc region amino acid variant has less than 50% starting binding as compared to an Fc region not containing the specific variant, with less than 70-80-90-95-98% loss of activity being preferred, and in general, with the activity being below the level of detectable binding in a Biacore assay. Of particular use in the ablation of FcγR binding is the double variant 236R/328R, and 236R and 328R separately as well.

By “ADCC” or “antibody dependent cell-mediated cytotoxicity” as used herein is meant the cell-mediated reaction wherein nonspecific cytotoxic cells that express FcγRs recognize bound antibody on a target cell and subsequently cause lysis of the target cell. ADCC is correlated with binding to FcγRIIIa; increased binding to FcγRIIIa leads to an increase in ADCC activity.

By “ADCP” or antibody dependent cell-mediated phagocytosis as used herein is meant the cell-mediated reaction wherein nonspecific cytotoxic cells that express FcγRs recognize bound antibody on a target cell and subsequently cause phagocytosis of the target cell.

By “CDC” or “complement dependent cytotoxicity” as used herein is meant the reaction wherein one or more complement protein components recognize bound antibody on a target cell and subsequently cause lysis of the target cell.

By “modification” herein is meant an amino acid substitution, insertion, and/or deletion in a polypeptide sequence or an alteration to a moiety chemically linked to a protein. For example, a modification may be an altered carbohydrate or PEG structure attached to a protein. By “amino acid modification” herein is meant an amino acid substitution, insertion, and/or deletion in a polypeptide sequence. For clarity, unless otherwise noted, the amino acid modification is always to an amino acid coded for by DNA, e.g. the 20 amino acids that have codons in DNA and RNA.

By “amino acid substitution” or “substitution” herein is meant the replacement of an amino acid at a particular position in a parent polypeptide sequence with a different amino acid. In particular, in some embodiments, the substitution is to an amino acid that is not naturally occurring at the particular position, either not naturally occurring within the organism or in any organism. For example, the substitution E272Y refers to a variant polypeptide, in this case an Fc variant, in which the glutamic acid at position 272 is replaced with tyrosine. For clarity, a protein which has been engineered to change the nucleic acid coding sequence but not change the starting amino acid (for example exchanging CGG (encoding arginine) to CGA (still encoding arginine) to increase host organism expression levels) is not an “amino acid substitution”; that is, despite the creation of a new gene encoding the same protein, if the protein has the same amino acid at the particular position that it started with, it is not an amino acid substitution.

By “amino acid insertion” or “insertion” as used herein is meant the addition of an amino acid sequence at a particular position in a parent polypeptide sequence. For example, -233E or −233E designates an insertion of glutamic acid after position 233 (the designated position) and before position 234. Additionally, -233ADE or ̂233ADE designates an insertion of AlaAspGlu after position 233 and before position 234.

By “amino acid deletion” or “deletion” as used herein is meant the removal of an amino acid sequence at a particular position in a parent polypeptide sequence. For example, E233- or E233# designates a deletion of glutamic acid at position 233. Additionally, EDA233- or EDA233# designates a deletion of the sequence GluAspAla that begins at position 233.

By “variant protein” or “protein variant”, or “variant” as used herein is meant a protein that differs from that of a parent protein by virtue of at least one amino acid modification. Protein variant may refer to the protein itself, a composition comprising the protein, or the amino sequence that encodes it. Preferably, the protein variant has at least one amino acid modification compared to the parent protein, e.g., from about one to about seventy amino acid modifications, and preferably from about one to about five amino acid modifications compared to the parent. As described below, in some embodiments the parent polypeptide, for example an Fc parent polypeptide, is a human wild type sequence, such as the Fc region from IgG1, IgG2, IgG3 or IgG4, although human sequences with variants can also serve as “parent polypeptides”. The protein variant sequence herein will preferably possess at least about 80% identity with a parent protein sequence, and most preferably at least about 90% identity, more preferably at least about 95-98-99% identity. Variant protein can refer to the variant protein itself, compositions comprising the protein variant, or the DNA sequence that encodes it. Accordingly, by “antibody variant” or “variant antibody” as used herein is meant an antibody that differs from a parent antibody by virtue of at least one amino acid modification, “IgG variant” or “variant IgG” as used herein is meant an antibody that differs from a parent IgG (again, in many cases, from a human IgG sequence) by virtue of at least one amino acid modification, and “immunoglobulin variant” or “variant immunoglobulin” as used herein is meant an immunoglobulin sequence that differs from that of a parent immunoglobulin sequence by virtue of at least one amino acid modification. “Fc variant” or “variant Fc” as used herein is meant a protein comprising an amino acid modification in an Fc domain. The Fc variants of the present invention are defined according to the amino acid modifications that compose them. Thus, for example, S239D or 239D is an Fc variant with the substitution aspartic acid at position 239 relative to the parent Fc polypeptide, wherein the numbering is according to the EU index. Likewise, S239D/I332E defines an Fc variant with the substitutions S239D and I332E relative to the parent Fc polypeptide. The identity of the WT amino acid may be unspecified, in which case the aforementioned variant is referred to as 239D/332E. It is noted that the order in which substitutions are provided is arbitrary, that is to say that, for example, 239D/332E is the same Fc variant as 332E/239D, and so on. For all positions discussed in the present invention that relate to antibodies, unless otherwise noted, amino acid position numbering is according to the EU index. The EU index or EU index as in Kabat or EU numbering scheme refers to the numbering of the EU antibody (Edelman et al., 1969, Proc Natl Acad Sci USA 63:78-85, hereby entirely incorporated by reference.) The modification can be an addition, deletion, or substitution. Substitutions can include naturally occurring amino acids and, in some cases, synthetic amino acids. Examples include U.S. Pat. No. 6,586,207; WO 98/48032; WO 03/073238; US2004-0214988A1; WO 05/35727A2; WO 05/74524A2; J. W. Chin et al., (2002), Journal of the American Chemical Society 124:9026-9027; J. W. Chin, & P. G. Schultz, (2002), ChemBioChem 11:1135-1137; J. W. Chin, et al., (2002), PICAS United States of America 99:11020-11024; and, L. Wang, & P. G. Schultz, (2002), Chem. 1-10, all entirely incorporated by reference.

As used herein, “protein” herein is meant at least two covalently attached amino acids, which includes proteins, polypeptides, oligopeptides and peptides. The peptidyl group may comprise naturally occurring amino acids and peptide bonds, or synthetic peptidomimetic structures, i.e., “analogs”, such as peptoids (see Simon et al., PNAS USA 89(20):9367 (1992), entirely incorporated by reference). The amino acids may either be naturally occurring or synthetic (e.g., not an amino acid that is coded for by DNA); as will be appreciated by those in the art. For example, homo-phenylalanine, citrulline, ornithine and noreleucine are considered synthetic amino acids for the purposes of the invention, and both D- and L-(R or S) configured amino acids may be utilized. The variants of the present invention may comprise modifications that include the use of synthetic amino acids incorporated using, for example, the technologies developed by Schultz and colleagues, including but not limited to methods described by Cropp & Shultz, 2004, Trends Genet. 20(12):625-30, Anderson et al., 2004, Proc Natl Acad Sci USA 101 (2):7566-71, Zhang et al., 2003, 303(5656):371-3, and Chin et al., 2003, Science 301(5635):964-7, all entirely incorporated by reference. In addition, polypeptides may include synthetic derivatization of one or more side chains or termini, glycosylation, PEGylation, circular permutation, cyclization, linkers to other molecules, fusion to proteins or protein domains, and addition of peptide tags or labels.

By “residue” as used herein is meant a position in a protein and its associated amino acid identity. For example, Asparagine 297 (also referred to as Asn297 or N297) is a residue at position 297 in the human antibody IgG1.

By “Fc” or “Fc region” or “Fc domain”, as used herein is meant the polypeptide comprising the constant region of an antibody excluding the first constant region immunoglobulin domain. Thus, Fc refers to the last two constant region immunoglobulin domains of IgA, IgD, and IgG, and the last three constant region immunoglobulin domains of IgE and IgM, and the flexible hinge N-terminal to these domains. For IgA and IgM, Fc may include the J chain. For IgG, Fc comprises immunoglobulin domains Cgamma2 and Cgamma3 (Cy2 and Cy3) and the hinge between Cgamma1 (Cy1) and Cgamma2 (Cy2). Although the boundaries of the Fc region may vary, the human IgG heavy chain Fc region is usually defined to comprise residues C226 or P230 to its carboxyl-terminus, wherein the numbering is according to the EU index as in Kabat. Fc may refer to this region in isolation, or this region in the context of an Fc polypeptide such as an antibody or immunoadhesin (e.g., an Fc fusion protein), as described below. It should be noted that for the purposes described herein, “Fc region” generally includes the hinge region, comprising residues 230-238, unless noted otherwise. Thus, an “Fc variant” can include variants of the hinge region, in the presence or absence of additional amino acid modifications in the Cy2 and Cy3 domains.

By “Fab” or “Fab region” as used herein is meant the polypeptide that comprises the VH, CHL VL, and CL immunoglobulin domains. Fab may refer to this region in isolation, or this region in the context of a full length antibody, antibody fragment or Fab fusion protein. By “Fv” or “Fv fragment” or “Fv region” as used herein is meant a polypeptide that comprises the VL and VH domains of a single antibody.

By “IgG subclass modification” or “isotype modification” as used herein is meant an amino acid modification that converts one amino acid of one IgG isotype to the corresponding amino acid in a different, aligned IgG isotype. For example, because IgG1 comprises a tyrosine and IgG2 a phenylalanine at EU position 296, a F296Y substitution in IgG2 is considered an IgG subclass modification.

By “non-naturally occurring modification” as used herein is meant an amino acid modification that is not isotypic. For example, because none of the IgGs comprise a serine at position 434, the substitution 434S in IgG1, IgG2, IgG3, or IgG4 (or hybrids thereof) is considered a non-naturally occurring modification.

By “amino acid” and “amino acid identity” as used herein is meant one of the 20 naturally occurring amino acids that are coded for by DNA and RNA.

By “Fc polypeptide” as used herein is meant a polypeptide that comprises all or part of an Fc region. Fc polypeptides include antibodies, Fc fusions (sometimes referred to as “Fc fusion proteins” or “immunoadhesins”), isolated Fcs, and Fc fragments.

By “effector function” as used herein is meant a biochemical event that results from the interaction of an antibody Fc region with an Fc receptor or ligand. Effector functions include but are not limited to ADCC, ADCP, and CDC.

By “effector cell” as used herein is meant a cell of the immune system that expresses one or more Fc receptors and mediates one or more effector functions. Effector cells include but are not limited to monocytes, macrophages, neutrophils, dendritic cells, eosinophils, mast cells, platelets, B cells, large granular lymphocytes, Langerhans' cells, natural killer (NK) cells, and γδ T cells, and may be from any organism including but not limited to humans, mice, rats, rabbits, and monkeys.

By “IgG Fc ligand” as used herein is meant a molecule, preferably a polypeptide, from any organism that binds to the Fc region of an IgG antibody to form an Fc/Fc ligand complex. Fc ligands include but are not limited to FcγRIs, FcγRIIs, FcγRIIIs, FcRn, C1q, C3, mannan binding lectin, mannose receptor, staphylococcal protein A, streptococcal protein G, and viral FcγR. Fc ligands also include Fc receptor homologs (FcRH), which are a family of Fc receptors that are homologous to the FcγRs (Davis et al., 2002, Immunological Reviews 190:123-136, entirely incorporated by reference). Fc ligands may include undiscovered molecules that bind Fc. Particular IgG Fc ligands are FcRn and Fc gamma receptors. By “Fc ligand” as used herein is meant a molecule, preferably a polypeptide, from any organism that binds to the Fc region of an antibody to form an Fc/Fc ligand complex.

By “Fc gamma receptor”, “FcγR” or “FcqammaR” as used herein is meant any member of the family of proteins that bind the IgG antibody Fc region and is encoded by an FcγR gene. In humans this family includes but is not limited to FcγRI (CD64), including isoforms FcγRIa, FcγRIb, and FcγRIc; FcγRII (CD32), including isoforms FcγRIIa (including allotypes H131 and R131), FcγRIIb (including FcγRIIb-1 and FcγRIIb-2), and FcγRIIc; and FcγRIII (CD16), including isoforms FcγRIIIa (including allotypes V158 and F158) and FcγRIIIb (including allotypes FcγRIIb-NA1 and FcγRIIb-NA2) (Jefferis et al., 2002, Immunol Lett 82:57-65, entirely incorporated by reference), as well as any undiscovered human FcγRs or FcγR isoforms or allotypes. An FcγR may be from any organism, including but not limited to humans, mice, rats, rabbits, and monkeys. Mouse FcγRs include but are not limited to FcγRI (CD64), FcγRII (CD32), FcγRIII (CD16), and FcγRIII-2 (CD16-2), as well as any undiscovered mouse FcγRs or FcγR isoforms or allotypes.

By “parent polypeptide” as used herein is meant a starting polypeptide that is subsequently modified to generate a variant. The parent polypeptide may be a naturally occurring polypeptide, or a variant or engineered version of a naturally occurring polypeptide. Parent polypeptide may refer to the polypeptide itself, compositions that comprise the parent polypeptide, or the amino acid sequence that encodes it. Accordingly, by “parent immunoglobulin” as used herein is meant an unmodified immunoglobulin polypeptide that is modified to generate a variant, and by “parent antibody” as used herein is meant an unmodified antibody that is modified to generate a variant antibody. It should be noted that “parent antibody” includes known commercial, recombinantly produced antibodies as outlined below.

By “Fc fusion protein” or “immunoadhesin” herein is meant a protein comprising an Fc region, generally linked (optionally through a linker moiety, as described herein) to a different protein, such as a binding moiety to a target protein, as described herein).

By “position” as used herein is meant a location in the sequence of a protein. Positions may be numbered sequentially, or according to an established format, for example the EU index for antibody numbering.

By “target antigen” as used herein is meant the molecule that is bound specifically by the variable region of a given antibody. A target antigen may be a protein, carbohydrate, lipid, or other chemical compound. A wide number of suitable target antigens are described below.

By “target cell” as used herein is meant a cell that expresses a target antigen.

By “variable region” as used herein is meant the region of an immunoglobulin that comprises one or more Ig domains substantially encoded by any of the V.kappa., V.lamda., and/or VH genes that make up the kappa, lambda, and heavy chain immunoglobulin genetic loci respectively.

By “wild type or WT” herein is meant an amino acid sequence or a nucleotide sequence that is found in nature, including allelic variations. A WT protein has an amino acid sequence or a nucleotide sequence that has not been intentionally modified.

The present invention is directed to the generation of antibodies containing Fc variants of the present invention.

### Antibodies

The present invention relates to the generation of antibodies, generally therapeutic antibodies, through the use of “Fc variants”. As is discussed below, the term “antibody” is used generally. Antibodies that find use in the present invention can take on a number of formats as described herein, including traditional antibodies as well as antibody derivatives, fragments and mimetics, described below. In general, the term “antibody” includes any polypeptide that includes at least one constant domain, including, but not limited to, CHL CH2, CH3 and CL.

Traditional antibody structural units typically comprise a tetramer. Each tetramer is typically composed of two identical pairs of polypeptide chains, each pair having one “light” (typically having a molecular weight of about 25 kDa) and one “heavy” chain (typically having a molecular weight of about 50-70 kDa). Human light chains are classified as kappa and lambda light chains. The present invention is directed to the IgG class, which has several subclasses, including, but not limited to IgG1, IgG2, IgG3, and IgG4. Thus, “isotype” as used herein is meant any of the subclasses of immunoglobulins defined by the chemical and antigenic characteristics of their constant regions. Examples of human isotypes include IgG1, IgG2, IgG3, IgG4, IgA1, IgA2, IgM, IgD, and IgE. It should be understood that therapeutic antibodies can also comprise hybrids of isotypes and/or subclasses. For example, as shown herein, the present invention covers antibodies that can contain one or both chains that are IgG1/G2 hybrids.

### Variants of the Invention

In general, as outlined above and unless noted otherwise, Fc variants include amino acid modifications in the hinge region and/or the Cγ2 and Cγ3 regions.

An Fc variant comprises one or more amino acid modifications relative to a parent Fc polypeptide, wherein the amino acid modification(s) optionally provide one or more optimized properties, although in some cases, the variants exhibit substantially identical biological properties. It should be recognized that “optimized” may include increases and/or decreases in biological activity. That is, as is outlined herein, it may be desirable in some cases to substantially ablate binding to one or more FcγRs selected from FcγRI, FcγRIIa, FcγRIIc, FcγRIIIa, and FcγRIIIb, even an activating receptor such as FcγIIIa.

An Fc variant of the present invention differs in amino acid sequence from its parent IgG by virtue of at least one amino acid modification. Thus, Fc variants of the present invention have at least one amino acid modification compared to the parent. Alternatively, the Fc variants of the present invention may have more than one amino acid modification as compared to the parent, for example from about one to fifty amino acid modifications, preferably from about one to ten amino acid modifications, and most preferably from about one to about five amino acid modifications compared to the parent. Thus, the sequences of the Fc variants and those of the parent Fc polypeptide are substantially homologous or identical. For example, the variant Fc variant sequences herein will possess about 80% homology (including identity) with the parent Fc variant sequence, preferably at least about 90% homology, and most preferably at least about 95, 96, 97, 98 and 99% identity. Modifications of the invention include amino acid modifications, including insertions, deletions, and substitutions. Modifications of the invention also include glycoform modifications. Modifications may be made genetically using molecular biology, or may be made enzymatically or chemically.

The Fc variants of the present invention are defined according to the amino acid modifications that compose them. Thus, for example, L328R is an Fc variant with the substitution L328R relative to the parent Fc polypeptide. Likewise, ̂236R/L328R defines an Fc variant with the insertion ̂236R and the substitution L328R relative to the parent Fc polypeptide. The identity of the WT amino acid may be unspecified, in which case the aforementioned variant is referred to as ̂236R/328R. It is noted that the order in which modifications are provided is arbitrary, that is to say that, for example, A236R/L328R is the same Fc variant as L328R/A236R, and so on. For all positions discussed in the present invention, numbering is according to the EU index or EU numbering scheme (Kabat et al., 1991, Sequences of Proteins of Immunological Interest, 5th Ed., United States Public Health Service, National Institutes of Health, Bethesda, hereby entirely incorporated by reference). The EU index or EU index as in Kabat or EU numbering scheme refers to the numbering of the EU antibody (Edelman et al., 1969, Proc Natl Acad Sci USA 63:78-85, hereby entirely incorporated by reference).

In one embodiment, one or more amino acid insertions are made. Amino acid insertions can be made within the hinge region, including at positions 233, 234, 235, 236 and 237. Exemplary insertions include, but are not limited to, ̂233L, ̂233EL, ̂234L, ̂235G, ̂235A, ̂235S, ̂235T, ̂235N, ̂235D, ̂235V, ̂235L, ̂235R, ̂237R, ̂237RR, ̂297G, ̂297D, ̂297A, ̂297S, ̂326G, ̂326T, ̂326D and ̂326E. Particular combinations of insertions and other modifications are also outlined in the figures. All of these may be done in any IgG molecule, particularly in IgG1 and IgG2. In some embodiments, insertions of glycine after position 235 are not preferred (A235G), except in combinations with other amino acid modifications.

In one embodiment, one or more amino acid deletions are made. Amino acid insertions can be made within the hinge region, including at positions 233, 234, 235, 236 and 237. Particular combinations of deletions and other modifications are also outlined in the figures. All of these may be done in any IgG molecule, particularly in IgG1 and IgG2. In some embodiments, deletions at position 236 are not preferred (236#), except in combinations with other amino acid modifications.

In one embodiment, one or more amino acid substitutions are made. Amino acid substitutions can be made at positions 221, 222, 224, 227, 228, 230, 231, 223, 233, 234, 235, 236, 237, 238, 239, 240, 241, 243, 244, 245, 246, 247, 249, 250, 258, 262, 263, 264, 265, 266, 267, 268, 269, 270, 271, 272, 273, 274, 275, 276, 278, 280, 281, 283, 285, 286, 288, 290, 291, 293, 294, 295, 296, 297, 298, 299, 300, 302, 313, 317, 318, 320, 322, 323, 324, 325, 326, 327, 328, 329, 330, 331, 332, 333, 334, 335 336 and 428, again, as any possible combination of substitution(s), insertion(s) and deletion(s). These amino acid substitutions include, but are not limited to, D221K, D221Y, K222E, K222Y, T223E, T223K, H224E, H224Y, T225E, T225K, T225W, P227E, P227G, P227K, P227Y, P228E, P228G, P228K, P228Y, P230A, P230E, P230G, P230Y, A231E, A231G, A231K, A231P, A231Y, P232E, P232G, P232K, P232Y, E233A, E233D, E233F, E233G, E233H, E233I, E233K, E233L, E233M, E233N, E233Q, E233R, E233S, E233T, E233V, E233W, E233Y, L234A, L234D, L234E, L234F, L234G, L234H, L234I, L234K, L234M, L234N, L234P, L234Q, L234R, L234S, L234T, L234V, L234W, L234Y, L235A, L235D, L235E, L235F, L235G, L235H, L235I, L235K, L235M, L235N, L235P, L235Q, L235R, L235S, L235T, L235V, L235W, L235Y, G236A, G236D, G236E, G236F, G236H, G236I, G236K, G236L, G236M, G236N, G236P, G236Q, G236R, G236S, G236T, G236V, G236W, G236Y, G237D, G237E, G237F, G237H, G237I, G237K, G237L, G237M, G237N, G237P, G237Q, G237R, G237S, G237T, G237V, G237W, G237Y, P238D, P238E, P238F, P238G, P238H, P238I, P238K, P238L, P238M, P238N, P238Q, P238R, P238S, P238T, P238V, P238W, P238Y, S239D, S239E, S239F, S239G, S239H, S239I, S239K, S239L, S239M, S239N, S239P, S239Q, S239R, S239T, S239V, S239W, S239Y, V240A, V240I, V240M, V240T, F241D, F241E, F241L, F241R, F241S, F241W, F241Y, F243E, F243H, F243L, F243Q, F243R, F243W, F243Y, P244H, P245A, K246D, K246E, K246H, K246Y, P247G, P247V, D249H, D249Q, D249Y, R255E, R255Y, E258H, E258S, E258Y, T260D, T260E, T260H, T260Y, V262A, V262E, V262F, V262I, V262T, V263A, V263I, V263M, V263T, V264A, V264D, V264E, V264F, V264G, V264H, V264I, V264K, V264L, V264M, V264N, V264P, V264Q, V264R, V264S, V264T, V264W, V264Y, D265F, D265G, D265H, D265I, D265K, D265L, D265M, D265N, D265P, D265Q, D265R, D265S, D265T, D265V, D265W, D265Y, V266A, V266I, V266M, V266T, S267D, S267E, S267F, S267H, S267I, S267K, S267L, S267M, S267N, S267P, S267Q, S267R, S267T, S267V, S267W, S267Y, H268D, H268E, H268F, H268G, H268I, H268K, H268L, H268M, H268P, H268Q, H268R, H268T, H268V, H268W, E269F, E269G, E269H, E269I, E269K, E269L, E269M, E269N, E269P, E269R, E269S, E269T, E269V, E269W, E269Y, D270F, D270G, D270H, D270I, D270L, D270M, D270P, D270Q, D270R, D270S, D270T, D270W, D270Y, P271A, P271D, P271E, P271F, P271G, P271H, P271I, P271K, P271L, P271M, P271N, P271Q, P271R, P271S, P271T, P271V, P271W, P271Y, E272D, E272F, E272G, E272H, E272I, E272K, E272L, E272M, E272P, E272R, E272S, E272T, E272V, E272W, E272Y, V273I, K274D, K274E, K274F, K274G, K274H, K274I, K274L, K274M, K274N, K274P, K274R, K274T, K274V, K274W, K274Y, F275L, F275W, N276D, N276E, N276F, N276G, N276H, N276I, N276L, N276M, N276P, N276R, N276S, N276T, N276V, N276W, N276Y, Y278D, Y278E, Y278G, Y278H, Y278I, Y278K, Y278L, Y278M, Y278N, Y278P, Y278Q, Y278R, Y278S, Y278T, Y278V, Y278W, D280G, D280K, D280L, D280P, D280W, G281D, G281E, G281K, G281N, G281P, G281Q, G281Y, V282E, V282G, V282K, V282P, V282Y, E283G, E283H, E283K, E283L, E283P, E283R, E283Y, V284D, V284E, V284L, V284N, V284Q, V284T, V284Y, H285D, H285E, H285K, H285Q, H285W, H285Y, N286E, N286G, N286P, N286Y, K288D, K288E, K288Y, K290D, K290H, K290L, K290N, K290W, P291D, P291E, P291G, P291H, P291I, P291Q, P291T, R292D, R292E, R292I, R292Y, E293F, E293G, E293H, E293I, E293L, E293M, E293N, E293P, E293R, E293S, E293T, E293V, E293W, E293Y, E294F, E294G, E294H, E294I, E294K, E294L, E294M, E294P, E294R, E294S, E294T, E294V, E294W, E294Y, Q295D, Q295E, Q295F, Q295G, Q295H, Q295I, Q295M, Q295N, Q295P, Q295R, Q295S, Q295T, Q295V, Q295W, Q295Y, Y296A, Y296D, Y296E, Y296G, Y296H, Y296I, Y296K, Y296L, Y296M, Y296N, Y296Q, Y296R, Y296S, Y296T, Y296V, N297D, N297E, N297F, N297G, N297H, N297I, N297K, N297L, N297M, N297P, N297Q, N297R, N297S, N297T, N297V, N297W, N297Y, S298D, S298E, S298F, S298H, S298I, S298K, S298M, S298N, S298Q, S298R, S298T, S298W, S298Y, T299A, T299D, T299E, T299F, T299G, T299H, T299I, T299K, T299L, T299M, T299N, T299P, T299Q, T299R, T299S, T299V, T299W, T299Y, Y300A, Y300D, Y300E, Y300G, Y300H, Y300K, Y300M, Y300N, Y300P, Y300Q, Y300R, Y300S, Y300T, Y300V, Y300W, R301D, R301E, R301H, R301Y, V302I, V303D, V303E, V303Y, S304D, S304H, S304L, S304N, S304T, V305E, V305T, V305Y, W313F, K317E, K317Q, E318H, E318L, E318Q, E318R, E318Y, K320D, K320F, K320G, K320H, K320I, K320L, K320N, K320P, K320S, K320T, K320V, K320W, K320Y, K322D, K322F, K322G, K322H, K322I, K322P, K322S, K322T, K322V, K322W, K322Y, V323I, S324D, S324F, S324G, S324H, 5324I, S324L, S324M, S324P, S324R, S324T, S324V, S324W, S324Y, N325A, N325D, N325E, N325F, N325G, N325H, N325I, N325K, N325L, N325M, N325P, N325Q, N325R, N325S, N325T, N325V, N325W, N325Y, K326I, K326L, K326P, K326T, A327D, A327E, A327F, A327H, A327I, A327K, A327L, A327M, A327N, A327P, A327R, A327S, A327T, A327V, A327W, A327Y, L328A, L328D, L328E, L328F, L328G, L328H, L328I, L328K, L328M, L328N, L328P, L328Q, L328R, L328S, L328T, L328V, L328W, L328Y, P329D, P329E, P329F, P329G, P329H, P329I, P329K, P329L, P329M, P329N, P329Q, P329R, P329S, P329T, P329V, P329W, P329Y, A330E, A330F, A330G, A330H, A330I, A330L, A330M, A330N, A330P, A330R, A330S, A330T, A330V, A330W, A330Y, P331D, P331F, P331H, P331I, P331L, P331M, P331Q, P331R, P331T, P331V, P331W, P331Y, I332A, I332D, 1332E, I332F, I332H, 1332K, I332L, I332M, I332N, I332P, I332Q, I332R, I332S, I332T, I332V, 1332W, I332Y, E333F, E333H, E333I, E333L, E333M, E333P, E333T, E333Y, K334F, K334I, K334L, K334P, K334T, T335D, T335F, T335G, T335H, T335I, T335L, T335M, T335N, T335P, T335R, T335S, T335V, T335W, T335Y, 1336E, 1336K, I336Y, S337E, S337H, and S337N, D221K, D221Y, K222E, K222Y, T223E, T223K, H224E, H224Y, T225E, T225K, T225W, P227E, P227G, P227K, P227Y, P228E, P228G, P228K, P228Y, P230A, P230A/E233D, P230A/E233D/I332E, P230E, P230G, P230Y, A231E, A231G, A231K, A231P, A231Y, P232E, P232G, P232K, P232Y, E233A, E233D, E233F, E233G, E233H, E233I, E233K, E233L, E233M, E233N, E233Q, E233R, E233S, E233T, E233V, E233W, E233Y, L234A, L234D, L234E, L234F, L234G, L234H, L234I, L234I/L235D, L234K, L234M, L234N, L234P, L234Q, L234R, L234S, L234T, L234V, L234W, L234Y, L235A, L235D, L235D/S239D/A330Y/I332E, L235D/S239D/N297D/I332E, L235E, L235F, L235G, L235H, L235I, L235K, L235M, L235N, L235P, L235Q, L235R, L235S, L235T, L235V, L235W, L235Y, G236A, G236D, G236E, G236F, G236H, G236I, G236K, G236L, G236M, G236N, G236P, G236Q, G236R, G236S, G236T, G236V, G236W, G236Y, G237D, G237E, G237F, G237H, G237I, G237K, G237L, G237M, G237N, G237P, G237Q, G237R, G237S, G237T, G237V, G237W, G237Y, P238D, P238E, P238F, P238G, P238H, P238I, P238K, P238L, P238M, P238N, P238Q, P238R, P238S, P238T, P238V, P238W, P238Y, S239D, S239D/A330L/I332E, S239D/A330Y/I332E/L234I, S239D/A330Y/I332E/V266I, S239D/D265F/N297D/I332E, S239D/D265H/N297D/I332E, S239D/D265I/N297D/I332E, S239D/D265L/N297D/I332E, S239D/D265T/N297D/I332E, S239D/D265Y/N297D/I332E, S239D/E272I/A330L/I332E, S239D/E272I/I332E, S239D/E272K/A330L/I332E, S239D/E272K/I332E, S239D/E272S/A330L/I332E, S239D/E272S/I332E, S239D/E272Y/A330L/I332E, S239D/E272Y/I332E, S239D/F241S/F243H/V262T/V264T/N297D/A330Y/I332E, S239D/H268D, S239D/H268E, S239D/I332D, S239D/I332E, S239D/I332E/A327D, S239D/I332E/A330I, S239D/I332E/A330Y, S239D/I332E/E272H, S239D/I332E/E272R, S239D/I332E/E283H, S239D/I332E/E283L, S239D/I332E/G236A, S239D/I332E/G236S, S239D/I332E/H268D, S239D/I332E/H268E, S239D/I332E/K246H, S239D/I332E/R255Y, S239D/I332E/S267E, S239D/I332E/V264I, S239D/I332E/V264I/A330L, S239D/I332E/V264I/S298A, S239D/I332E/V284D, S239D/I332E/V284E, S239D/I332E/V284E, S239D/I332N, S239D/I332Q, S239D/K274E/A330L/I332E, S239D/K274E/I332E, S239D/K326E/A330L/I332E, S239D/K326E/A330Y/I332E, S239D/K326E/I332E, S239D/K326T/A330Y/I332E, S239D/K326T/I332E, S239D/N297D/A330Y/I332E, S239D/N297D/I332E, S239D/N297D/K326E/I332E, S239D/S267E/A330L/I332E, S239D/S267E/I332E, S239D/S298A/K326E/I332E, S239D/S298A/K326T/I332E, S239D/V240I/A330Y/I332E, S239D/V264T/A330Y/I332E, S239D/Y278T/A330L/I332E, S239D/Y278T/I332E, S239E, S239E/D265G, S239E/D265N, S239E/D265Q, S239E/I332D, S239E/I332E, S239E/I332N, S239E/I332Q, S239E/N297D/I332E, S239E/V264I/A330Y/I332E, S239E/V264I/I332E, S239E/V264I/S298A/A330Y/I332E, S239F, S239G, S239H, S239I, S239K, S239L, S239M, S239N, S239N/I332D, S239N/I332E, S239N/I332E/A330L, S239N/I332E/A330Y, S239N/I332N, S239N/I332Q, S239P, S239Q, S239Q/I332D, S239Q/I332E, S239Q/I332N, S239Q/I332Q, S239Q/V264I/I332E, S239R, S239T, S239V, S239W, S239Y, V240A, V240I, V240I/V266I, V240M, V240T, F241D, F241E, F241E/F243Q/V262T/V264E/I332E, F241E/F243Q/V262T/V264E, F241E/F243R/V262E/V264R/I332E, F241E/F243R/V262E/V264R, F241E/F243Y/V262T/V264R/I332E, F241E/F243Y/C262T/V264R, F241L, F241L/F243L/V262I/V264I, F241L/V262I, F241R/F243Q/V262T/V264R/I332E, F241R/F243Q/V262T/V264R, F241W, F241W/F243W, F241W/F243W/V262A/V264A, F241Y, F241Y/F243Y/V262T/V264T/N297D/I332E, F241Y/F243Y/V262T/V264T, F243E, F243L, F243L/V262I/N264W, F243L/V264I, F243W, P244H, P244H/P245A/P247V, P245A, K246D, K246E, K246H, K246Y, P247G, P247V, D249H, D249Q, D249Y, R255E, R255Y, E258H, E258S, E258Y, T260D, T260E, T260H, T260Y, V262E, V262F, V263A, V263I, V263M, V263T, V264A, V264D, V264E, V264E/N297D/I332E, V264F, V264G, V264H, V264I, V264I/A330L/I332E, V264I/A330Y/I332E, V264I/I332E, V264K, V264L, V264M, V264N, V264P, V264Q, V264R, V264S, V264T, V264W, V264Y, D265F, D265F/N297E/I332E, D265G, D265H, D265I, D265K, D265L, D265M, D265N, D265P, D265Q, D265R, D265S, D265T, D265V, D265W, D265Y, D265Y/N297D/I332E, D265Y/N297D/T299L/I332E, V266A, V266I, V266M, V266T, S267D, S267E, S267E, S267E/A327D, S267E/P331D, S267E/S324I, S267E/V282G, S267F, S267H, S267I, S267K, S267L, S267L/A327S, S267M, S267N, S267P, S267Q, S267Q/A327S, S267R, S267T, S267V, S267W, S267Y, H268D, H268E, H268F, H268G, H268I, H268K, H268L, H268M, H268P, H268Q, H268R, H268T, H268V, H268W, E269F, E269G, E269H, E269I, E269K, E269L, E269M, E269N, E269P, E269R, E269S, E269T, E269V, E269W, E269Y, D270F, D270G, D270H, D270I, D270L, D270M, D270P, D270Q, D270R, D270S, D270T, D270W, D270Y, P271A, P271D, P271E, P271F, P271G, P271H, P271I, P271K, P271L, P271M, P271N, P271Q, P271R, P271S, P271T, P271V, P271W, P271Y, E272D, E272F, E272G, E272H, E272I, E272K, E272L, E272M, E272P, E272R, E272S, E272T, E272V, E272W, E272Y, V273I, K274D, K274E, K274F, K274G, K274H, K274I, K274L, K274M, K274N, K274P, K274R, K274T, K274V, K274W, K274Y, F275L, F275W, N276D, N276E, N276F, N276G, N276H, N276I, N276L, N276M, N276P, N276R, N276S, N276T, N276V, N276W, N276Y, Y278D, Y278E, Y278G, Y278H, Y278I, Y278K, Y278L, Y278M, Y278N, Y278P, Y278Q, Y278R, Y278S, Y278T, Y278V, Y278W, Y278W, Y278W/E283R/V302I, Y278W/V302I, D280G, D280K, D280L, D280P, D280W, G281D, G281D/V282G, G281E, G281K, G281N, G281P, G281Q, G281Y, V282E, V282G, V282G/P331D, V282K, V282P, V282Y, E283G, E283H, E283K, E283L, E283P, E283R, E283R/V302I/Y278W/E283R, E283Y, V284D, V284E, V284L, V284N, V284Q, V284T, V284Y, H285D, H285E, H285K, H285Q, H285W, H285Y, N286E, N286G, N286P, N286Y, K288D, K288E, K288Y, K290D, K290H, K290L, K290N, K290W, P291D, P291E, P291G, P291H, P291I, P291Q, P291T, R292D, R292E, R292T, R292Y, E293F, E293G, E293H, E293I, E293L, E293M, E293N, E293P, E293R, E293S, E293T, E293V, E293W, E293Y, E294F, E294G, E294H, E294I, E294K, E294L, E294M, E294P, E294R, E294S, E294T, E294V, E294W, E294Y, Q295D, Q295E, Q295F, Q295G, Q295H, Q295I, Q295M, Q295N, Q295P, Q295R, Q295S, Q295T, Q295V, Q295W, Q295Y, Y296A, Y296D, Y296E, Y296G, Y296I, Y296K, Y296L, Y296M, Y296N, Y296Q, Y296R, Y296S, Y296T, Y296V, N297D, N297D/I332E, N297D/I332E/A330Y, N297D/I332E/S239D/A330L, N297D/I332E/S239D/D265V, N297D/I332E/S298A/A330Y, N297D/I332E/T299E, N297D/I332E/T299F, N297D/I332E/T299H, N297D/I332E/T299I, N297D/I332E/T299L, N297D/I332E/T299V, N297D/I332E/V296D, N297D/I332E/Y296E, N297D/I332E/Y296H, N297D/I332E/Y296N, N297D/I332E/Y296Q, N297D/I332E/Y296T, N297E/I332E, N297F, N297G, N297H, N297I, N297K, N297L, N297M, N297P, N297Q, N297R, N297S, N297S/I332E, N297T, N297V, N297W, N297Y, S298A/I332E, S298A/K326E, S298A/K326E/K334L, S298A/K334L, S298D, S298E, S298F, S298H, 5298I, S298K, S298M, S298N, S298Q, S298R, S298T, S298W, S298Y, T299A, T299D, T299E, T299F, T299G, T299H, T299I, T299K, T299L, T299M, T299N, T299P, T299Q, T299R, T299S, T299V, T299W, T299Y, Y300A, Y300D, Y300E, Y300G, Y300H, Y300K, Y300M, Y300N, Y300P, Y300Q, Y300R, Y300S, Y300T, Y300V, Y300W, R301D, R301E, R301H, R301Y, V302I, V303D, V303E, V303Y, S304D, S304H, S304L, S304N, S304T, V305E, V305T, V305Y, W313F, K317E, K317Q, E318H, E318L, E318Q, E318R, E318Y, K320D, K320F, K320G, K320H, K320I, K320L, K320N, K320P, K320S, K320T, K320V, K320W, K320Y, K322D, K322F, K322G, K322H, K322I, K322P, K322S, K322T, K322V, K322W, K322Y, V323I, S324D, S324F, S324G, S324H, S324I, S324I/A327D, S324L, S324M, S324P, S324R, S324T, S324V, S324W, S324Y, N325A, N325D, N325E, N325F, N325G, N325H, N325I, N325K, N325L, N325M, N325P, N325Q, N325R, N325S, N325T, N325V, N325W, N325Y, K326I, K326L, K326P, K326T, A327D, A327E, A327F, A327H, A327I, A327K, A327L, A327M, A327N, A327P, A327R, A327S, A327T, A327V, A327W, A327Y, L328A, L328D, L328D/I332E, L328E, L328E/I332E, L328F, L328G, L328H, L328H/I332E, L328I, L328I/I332E, L328I/I332E, L328K, L328M, L328M/I332E, L328N, L328N/I332E, L328P, L328Q, L328Q/I332E, L328Q/I332E, L328R, L328S, L328T, L328T/I332E, L328V, L328V/I332E, L328W, L328Y, P329D, P329E, P329F, P329G, P329H, P329I, P329K, P329L, P329M, P329N, P329Q, P329R, P329S, P329T, P329V, P329W, P329Y, A330E, A330F, A330G, A330H, A330I, A330L, A330L/I332E, A330M, A330N, A330P, A330R, A330S, A330T, A330V, A330W, A330Y, A330Y/I332E, P331D, P331F, P331H, P331I, P331L, P331M, P331Q, P331R, P331T, P331V, P331W, P331Y, I332A, I332D, 1332E, I332E/G281D, I332E/H268D, I332E/H268E, I332E/S239D/S298A, I332E/S239N/S298A, I332E/V264I/S298A, I332E/V284E, I332F, I332H, I332K, I332L, I332M, I332N, I332P, I332Q, I332R, I332S, I332T, I332V, I332W, I332Y, E333F, E333H, E333I, E333L, E333M, E333P, E333T, E333Y, K334F, K334I, K334P, K334T, T335D, T335F, T335G, T335H, T335I, T335L, T335M, T335N, T335P, T335R, T335S, T335V, T335W, T335Y, 1336E, I336K, I336Y, S337E, S337H, and S337N, wherein the numbering of the residues in the Fc region is that of the EU index as in Kabat, as is true throughout. Particular combinations of insertion(s), deletion(s) and other modifications are also outlined in the figures. All of these may be done in any IgG molecule, particularly in IgG1 and IgG2.

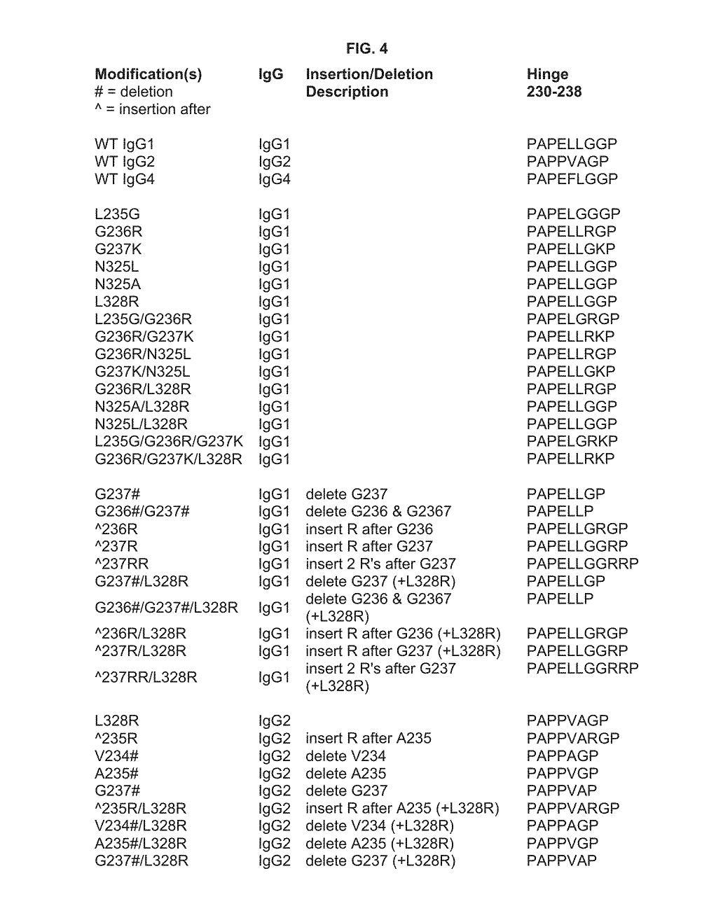

In some embodiments, combinations of modifications that find use in the present invention are found in FIGS. 4 and 6-17, and additionally include ̂236R/L328R (particularly in IgG1) and ̂236A/I332E (particularly in IgG2). Similarly, amino modifications at 332 and/or 239 can be coupled with insertion(s) and/or deletion(s).

Functionally, variants that result in increased binding to activating FcγRs as compared to the change in binding affinity to inhibitory FcγRs find particular use in some embodiments.

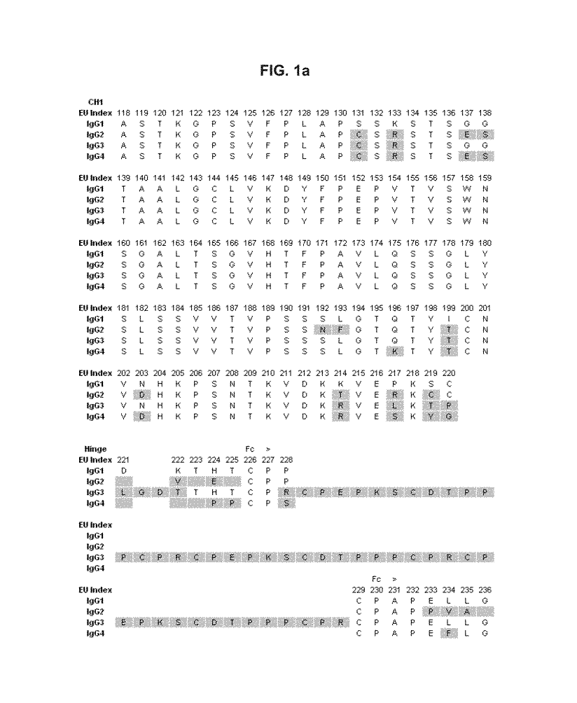

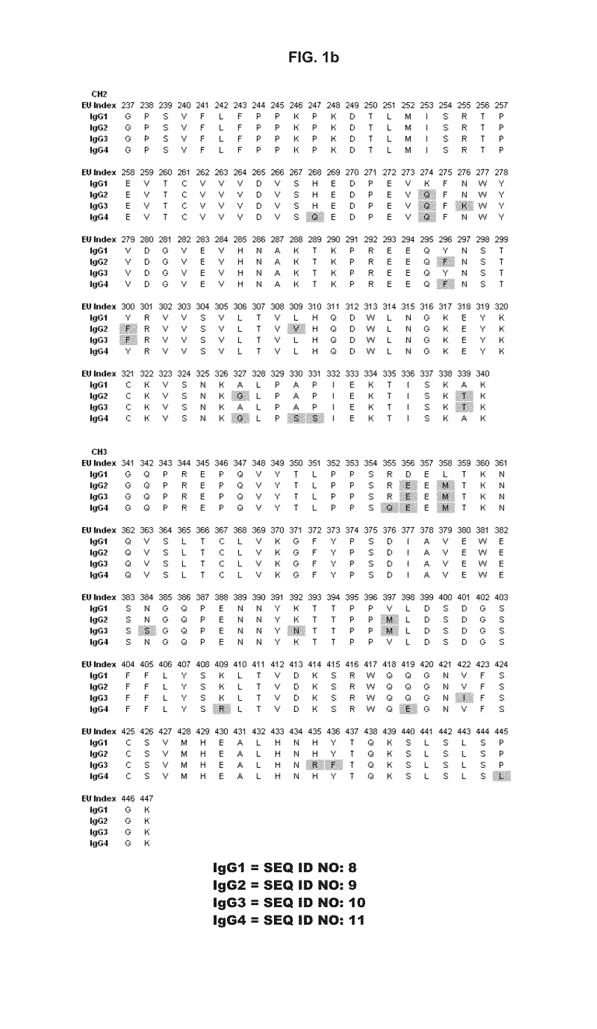

The Fc variants of the present invention may be substantially encoded by immunoglobulin genes belonging to any of the antibody classes. In certain embodiments, the Fc variants of the present invention find use in antibodies or Fc fusions that comprise sequences belonging to the IgG class of antibodies, including IgG1, IgG2, IgG3, or IgG4. FIG. 1 provides an alignment of these human IgG sequences. In an alternate embodiment the Fc variants of the present invention find use in antibodies or Fc fusions that comprise sequences belonging to the IgA (including subclasses IgA1 and IgA2), IgD, IgE, IgG, or IgM classes of antibodies. The Fc variants of the present invention may comprise more than one protein chain. That is, the present invention may find use in an antibody or Fc fusion that is a monomer or an oligomer, including a homo- or hetero-oligomer.

In certain embodiments, the Fc variants of the invention are based on human IgG sequences, e.g., IgG1, IgG2, IgG3, IgG4, and thus human IgG sequences are used as the “base” sequences against which other sequences are compared, including but not limited to sequences from other organisms, for example rodent and primate sequences. Fc variants may also comprise sequences from other immunoglobulin classes such as IgA, IgE, IgGD, IgGM, and the like. It is contemplated that, although the Fc variants of the present invention are engineered in the context of one parent IgG, the variants may be engineered in or “transferred” to the context of another, second parent IgG. This is done by determining the “equivalent” or “corresponding” residues and substitutions between the first and second IgG, typically based on sequence or structural homology between the sequences of the first and second IgGs. In order to establish homology, the amino acid sequence of a first IgG outlined herein is directly compared to the sequence of a second IgG. After aligning the sequences, using one or more of the homology alignment programs known in the art (for example using conserved residues as between species), allowing for necessary insertions and deletions in order to maintain alignment (i.e., avoiding the elimination of conserved residues through arbitrary deletion and insertion), the residues equivalent to particular amino acids in the primary sequence of the first Fc variant are defined. Alignment of conserved residues preferably should conserve 100% of such residues. However, alignment of greater than 75% or as little as 50% of conserved residues is also adequate to define equivalent residues. Equivalent residues may also be defined by determining structural homology between a first and second IgG that is at the level of tertiary structure for IgGs whose structures have been determined. In this case, equivalent residues are defined as those for which the atomic coordinates of two or more of the main chain atoms of a particular amino acid residue of the parent or precursor (N on N, CA on CA, C on C and O on O) are within about 0.13 nm and preferably about 0.1 nm after alignment. Alignment is achieved after the best model has been oriented and positioned to give the maximum overlap of atomic coordinates of non-hydrogen protein atoms of the proteins. Regardless of how equivalent or corresponding residues are determined, and regardless of the identity of the parent IgG in which the IgGs are made, what is meant to be conveyed is that the Fc variants discovered by the present invention may be engineered into any second parent IgG that has significant sequence or structural homology with the Fc variant. Thus, for example, if a variant antibody is generated wherein the parent antibody is human IgG1, by using the methods described above or other methods for determining equivalent residues, the variant antibody may be engineered in another IgG1 parent antibody that binds a different antigen, a human IgG2 parent antibody, a human IgA parent antibody, a mouse IgG2a or IgG2b parent antibody, a recombinant IgG1/IgG2 antibody and the like. Again, as described above, the context of the parent Fc variant does not affect the ability to transfer the Fc variants of the present invention to other parent IgGs. In this manner, the present invention contemplates the generation of a repertoire of antibodies having an Fc region, with one or more modifications described herein, in the context of different IgG scaffolds (represented by FIG. 137). This “transferability” occurs not only across different IgG isotypes but also across different antigen specificities. For instance, Fc variants of the present invention may be engineered and combined with different Fv regions such that the Fc effector function and Fv antigen specificity are independently retained.

Fc variants of the present invention may be substantially encoded by genes from any organism, preferably mammals, including but not limited to humans, rodents including but not limited to mice and rats, lagomorpha including but not limited to rabbits and hares, camelidae including but not limited to camels, llamas, and dromedaries, and non-human primates, including but not limited to Prosimians, Platyrrhini (New World monkeys), Cercopithecoidea (Old World monkeys), and Hominoidea including the Gibbons and Lesser and Great Apes. In a certain embodiments, the Fc variants of the present invention are substantially human.

As is well known in the art, immunoglobulin polymorphisms exist in the human population. Gm polymorphism is determined by the IGHG1, IGHG2 and IGHG3 genes which have alleles encoding allotypic antigenic determinants referred to as G1m, G2m, and G3m allotypes for markers of the human IgG1, IgG2 and IgG3 molecules (no Gm allotypes have been found on the gamma 4 chain). Markers may be classified into ‘allotypes’ and ‘isoallotypes’. These are distinguished on different serological bases dependent upon the strong sequence homologies between isotypes. Allotypes are antigenic determinants specified by allelic forms of the Ig genes. Allotypes represent slight differences in the amino acid sequences of heavy or light chains of different individuals. Even a single amino acid difference can give rise to an allotypic determinant, although in many cases there are several amino acid substitutions that have occurred. Allotypes are sequence differences between alleles of a subclass whereby the antisera recognize only the allelic differences. An isoallotype is an allele in one isotype which produces an epitope which is shared with a non-polymorphic homologous region of one or more other isotypes and because of this the antisera will react with both the relevant allotypes and the relevant homologous isotypes (Clark, 1997, IgG effector mechanisms, Chem Immunol. 65:88-110; Gorman & Clark, 1990, Semin Immunol 2(6):457-66, both hereby entirely incorporated by reference).

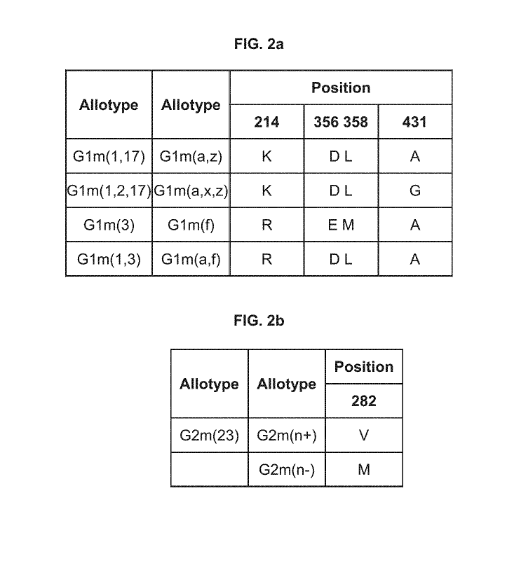

Allelic forms of human immunoglobulins have been well-characterized (WHO Review of the notation for the allotypic and related markers of human immunoglobulins. J Immunogen 1976, 3: 357-362; WHO Review of the notation for the allotypic and related markers of human immunoglobulins. 1976, Eur. J. Immunol. 6, 599-601; Loghem E van, 1986, Allotypic markers, Monogr Allergy 19: 40-51, all hereby entirely incorporated by reference). Additionally, other polymorphisms have been characterized (Kim et al., 2001, J. Mol. Evol. 54:1-9, hereby entirely incorporated by reference). At present, 18 Gm allotypes are known: G1m (1, 2, 3, 17) or G1m (a, x, f, z), G2m (23) or G2m (n), G3m (5, 6, 10, 11, 13, 14, 15, 16, 21, 24, 26, 27, 28) or G3m (b1, c3, b5, b0, b3, b4, s, t, g1, c5, u, v, g5) (Lefranc, et al., The human IgG subclasses: molecular analysis of structure, function and regulation. Pergamon, Oxford, pp. 43-78 (1990); Lefranc, G. et al., 1979, Hum. Genet.: 50, 199-211, both hereby entirely incorporated by reference). Allotypes that are inherited in fixed combinations are called Gm haplotypes. FIG. 2 shows common haplotypes of the gamma chain of human IgG1 (FIG. 2a) and IgG2 (FIG. 2b) showing the positions and the relevant amino acid substitutions. The Fc variants of the present invention may be substantially encoded by any allotype, isoallotype, or haplotype of any immunoglobulin gene.

In some embodiments, the present invention provides antibodies having a heavy chain and a light chain, wherein the heavy chain contains a sequence selected from the sixteen sequences (>1 to >16) shown in FIG. 136A. In some embodiments, the present invention provides an antibody having a heavy chain and a light chain, wherein the heavy chain contains sequence >1 shown in FIG. 136A and the antibody is specific for CD30. In some embodiments, the present invention provides an antibody having a heavy chain and a light chain, wherein the heavy chain contains sequence >2 shown in FIG. 136A and the antibody is specific for CD19. In some embodiments, the present invention provides an antibody having a heavy chain and a light chain, wherein the heavy chain contains sequence >3 shown in FIG. 136A and the antibody is specific for CD40. In some embodiments, the present invention provides an antibody having a heavy chain and a light chain, wherein the heavy chain contains sequence >4 shown in FIG. 136A and the antibody is specific for HM1.24. In some embodiments, the present invention provides an antibody having a heavy chain and a light chain, wherein the heavy chain contains sequence >5 shown in FIG. 136A and the antibody is specific for CD19. In some embodiments, the present invention provides an antibody having a heavy chain and a light chain, wherein the heavy chain contains sequence >6 shown in FIG. 136A and the antibody is specific for IgE. In some embodiments, the present invention provides an antibody having a heavy chain and a light chain, wherein the heavy chain contains sequence >7 shown in FIG. 136A and the antibody is specific for VEGF. In some embodiments, the present invention provides an antibody having a heavy chain and a light chain, wherein the heavy chain contains sequence >8 shown in FIG. 136A and the antibody is specific for IgE. In some embodiments, the present invention provides an antibody having a heavy chain and a light chain, wherein the heavy chain contains sequence >9 shown in FIG. 136A and the antibody is specific for TNF. In some embodiments, the present invention provides an antibody having a heavy chain and a light chain, wherein the heavy chain contains sequence >10 shown in FIG. 136A and the antibody is specific for EGFR. In some embodiments, the present invention provides an antibody having a heavy chain and a light chain, wherein the heavy chain contains sequence >11 shown in FIG. 136A and the antibody is specific for EGFR. In some embodiments, the present invention provides an antibody having a heavy chain and a light chain, wherein the heavy chain contains sequence >12 shown in FIG. 136A and the antibody is specific for EGFR. In some embodiments, the present invention provides an antibody having a heavy chain and a light chain, wherein the heavy chain contains sequence >13 shown in FIG. 136A and the antibody is specific for EGFR. In some embodiments, the present invention provides an antibody having a heavy chain and a light chain, wherein the heavy chain contains sequence >14 shown in FIG. 136A and the antibody is specific for CD20. In some embodiments, the present invention provides an antibody having a heavy chain and a light chain, wherein the heavy chain contains sequence >15 shown in FIG. 136A and the antibody is specific for CD20. In some embodiments, the present invention provides an antibody having a heavy chain and a light chain, wherein the heavy chain contains sequence >16 shown in FIG. 136A and the antibody is specific for CD20.

In other embodiments, the present invention provides antibodies having a heavy chain and a light chain, wherein the light chain contains a sequence selected from the sixteen sequences shown in FIG. 136B. In some embodiments, the present invention provides an antibody having a heavy chain and a light chain, wherein the light chain contains sequence >1 shown in FIG. 136B and the antibody is specific for CD30. In some embodiments, the present invention provides an antibody having a heavy chain and a light chain, wherein the light chain contains sequence >2 shown in FIG. 136B and the antibody is specific for CD19. In some embodiments, the present invention provides an antibody having a heavy chain and a light chain, wherein the light chain contains sequence >3 shown in FIG. 136B and the antibody is specific for CD40. In some embodiments, the present invention provides an antibody having a heavy chain and a light chain, wherein the light chain contains sequence >4 shown in FIG. 136B and the antibody is specific for HM1.24. In some embodiments, the present invention provides an antibody having a heavy chain and a light chain, wherein the light chain contains sequence >5 shown in FIG. 136B and the antibody is specific for CD19. In some embodiments, the present invention provides an antibody having a heavy chain and a light chain, wherein the light chain contains sequence >6 shown in FIG. 136B and the antibody is specific for IgE. In some embodiments, the present invention provides an antibody having a heavy chain and a light chain, wherein the light chain contains sequence >7 shown in FIG. 136B and the antibody is specific for VEGF. In some embodiments, the present invention provides an antibody having a heavy chain and a light chain, wherein the light chain contains sequence >8 shown in FIG. 136B and the antibody is specific for IgE. In some embodiments, the present invention provides an antibody having a heavy chain and a light chain, wherein the light chain contains sequence >9 shown in FIG. 136B and the antibody is specific for TNF. In some embodiments, the present invention provides an antibody having a heavy chain and a light chain, wherein the light chain contains sequence >10 shown in FIG. 136B and the antibody is specific for EGFR. In some embodiments, the present invention provides an antibody having a heavy chain and a light chain, wherein the light chain contains sequence >11 shown in FIG. 136B and the antibody is specific for EGFR. In some embodiments, the present invention provides an antibody having a heavy chain and a light chain, wherein the light chain contains sequence >12 shown in FIG. 136B and the antibody is specific for EGFR. In some embodiments, the present invention provides an antibody having a heavy chain and a light chain, wherein the light chain contains sequence >13 shown in FIG. 136B and the antibody is specific for EGFR. In some embodiments, the present invention provides an antibody having a heavy chain and a light chain, wherein the light chain contains sequence >14 shown in FIG. 136B and the antibody is specific for CD20. In some embodiments, the present invention provides an antibody having a heavy chain and a light chain, wherein the light chain contains sequence >15 shown in FIG. 136B and the antibody is specific for CD20. In some embodiments, the present invention provides an antibody having a heavy chain and a light chain, wherein the light chain contains sequence >16 shown in FIG. 136B and the antibody is specific for CD20.

In still other embodiments, the present invention provides antibodies having a heavy chain and a light chain, wherein the heavy chain sequence contains sequence >1 of FIG. 136A and the light chain contains a sequence selected from sequences >1, >2, >3, >4, >5, >6, >7, >8, >9, >10, >11, >12, >13, >14, >15, and >16 of FIG. 136B. In still other embodiments, the present invention provides antibodies having a heavy chain and a light chain, wherein the heavy chain contains sequence >2 of FIG. 136A and the light chain contains a sequence selected from sequences >1, >2, >3, >4, >5, >6, >7, >8, >9, >10, >11, >12, >13, >14, >15, and >16 of FIG. 136B. In still other embodiments, the present invention provides antibodies having a heavy chain and a light chain, wherein the heavy chain contains sequence >3 of FIG. 136A and the light chain contains a sequence selected from sequences >1, >2, >3, >4, >5, >6, >7, >8, >9, >10, >11, >12, >13, >14, >15, and >16 of FIG. 136B. In still other embodiments, the present invention provides antibodies having a heavy chain and a light chain, wherein the heavy chain contains sequence >4 of FIG. 136A and the light chain contains a sequence selected from sequences >1, >2, >3, >4, >5, >6, >7, >8, >9, >10, >11, >12, >13, >14, >15, and >16 of FIG. 136B. In still other embodiments, the present invention provides antibodies having a heavy chain and a light chain, wherein the heavy chain contains sequence >5 of FIG. 136A and the light chain contains a sequence selected from sequences >1, >2, >3, >4, >5, >6, >7, >8, >9, >10, >11, >12, >13, >14, >15, and >16 of FIG. 136B. In still other embodiments, the present invention provides antibodies having a heavy chain and a light chain, wherein the heavy chain contains sequence >6 of FIG. 136A and the light chain contains a sequence selected from sequences >1, >2, >3, >4, >5, >6, >7, >8, >9, >10, >11, >12, >13, >14, >15, and >16 of FIG. 136B. In still other embodiments, the present invention provides antibodies having a heavy chain and a light chain, wherein the heavy chain contains sequence >7 of FIG. 136A and the light chain contains a sequence selected from sequences >1, >2, >3, >4, >5, >6, >7, >8, >9, >10, >11, >12, >13, >14, >15, and >16 of FIG. 136B. In still other embodiments, the present invention provides antibodies having a heavy chain and a light chain, wherein the heavy chain contains sequence >8 of FIG. 136A and the light chain contains a sequence selected from sequences >1, >2, >3, >4, >5, >6, >7, >8, >9, >10, >11, >12, >13, >14, >15, and >16 of FIG. 136B. In still other embodiments, the present invention provides antibodies having a heavy chain and a light chain, wherein the heavy chain contains sequence >9 of FIG. 136A and the light chain contains a sequence selected from sequences >1, >2, >3, >4, >5, >6, >7, >8, >9, >10, >11, >12, >13, >14, >15, and >16 of FIG. 136B. In still other embodiments, the present invention provides antibodies having a heavy chain and a light chain, wherein the heavy chain contains sequence >10 of FIG. 136A and the light chain contains a sequence selected from sequences >1, >2, >3, >4, >5, >6, >7, >8, >9, >10, >11, >12, >13, >14, >15, and >16 of FIG. 136B. In still other embodiments, the present invention provides antibodies having a heavy chain and a light chain, wherein the heavy chain contains sequence >11 of FIG. 136A and the light chain contains a sequence selected from sequences >1, >2, >3, >4, >5, >6, >7, >8, >9, >10, >11, >12, >13, >14, >15, and >16 of FIG. 136B. In still other embodiments, the present invention provides antibodies having a heavy chain and a light chain, wherein the heavy chain contains sequence >12 of FIG. 136A and the light chain contains a sequence selected from sequences >1, >2, >3, >4, >5, >6, >7, >8, >9, >10, >11, >12, >13, >14, >15, and >16 of FIG. 136B. In still other embodiments, the present invention provides antibodies having a heavy chain and a light chain, wherein the heavy chain contains sequence >13 of FIG. 136A and the light chain contains a sequence selected from sequences >1, >2, >3, >4, >5, >6, >7, >8, >9, >10, >11, >12, >13, >14, >15, and >16 of FIG. 136B. In still other embodiments, the present invention provides antibodies having a heavy chain and a light chain, wherein the heavy chain contains sequence >14 of FIG. 136A and the light chain contains a sequence selected from sequences >1, >2, >3, >4, >5, >6, >7, >8, >9, >10, >11, >12, >13, >14, >15, and >16 of FIG. 136B. In still other embodiments, the present invention provides antibodies having a heavy chain and a light chain, wherein the heavy chain contains sequence >15 of FIG. 136A and the light chain contains a sequence selected from sequences >1, >2, >3, >4, >5, >6, >7, >8, >9, >10, >11, >12, >13, >14, >15, and >16 of FIG. 136B. In still other embodiments, the present invention provides antibodies having a heavy chain and a light chain, wherein the heavy chain contains sequence >16 of FIG. 136A and the light chain contains a sequence selected from sequences >1, >2, >3, >4, >5, >6, >7, >8, >9, >10, >11, >12, >13, >14, >15, and >16 of FIG. 136B.

The amino-terminal portion of each chain includes a variable region of about 100 to 110 or more amino acids primarily responsible for antigen recognition, generally referred to in the art and herein as the “Fv domain” or “Fv region”. In the variable region, three loops are gathered for each of the V domains of the heavy chain and light chain to form an antigen-binding site. Each of the loops is referred to as a complementarity-determining region (hereinafter referred to as a “CDR”), in which the variation in the amino acid sequence is most significant. “Variable” refers to the fact that certain segments of the variable region differ extensively in sequence among antibodies. Variability within the variable region is not evenly distributed. Instead, the V regions consist of relatively invariant stretches called framework regions (FRs) of 15-30 amino acids separated by shorter regions of extreme variability called “hypervariable regions” that are each 9-15 amino acids long or longer.

In some embodiments, the present invention provides antibodies having a VH domain and a VL domain, wherein the VH domain contains the variable region of a sequence selected from the sixteen sequences (>1 to >16) shown in FIG. 136A. In some embodiments, antibodies are provided having a VH domain and a VL domain, wherein the VH domain contains the variable region of sequence >1 shown in FIG. 136A and the antibody is specific for CD30. In some embodiments, antibodies are provided having a VH domain and a VL domain, wherein the VH domain contains the variable region of sequence >2 shown in FIG. 136A and the antibody is specific for CD19. In some embodiments, antibodies are provided having a VH domain and a VL domain, wherein the VH domain contains the variable region of sequence >3 shown in FIG. 136A and the antibody is specific for CD40. In some embodiments, antibodies are provided having a VH domain and a VL domain, wherein the VH domain contains the variable region of sequence >4 shown in FIG. 136A and the antibody is specific for HM1.24. In some embodiments, antibodies are provided having a VH domain and a VL domain, wherein the VH domain contains the variable region of sequence >5 shown in FIG. 136A and the antibody is specific for CD19. In some embodiments, antibodies are provided having a VH domain and a VL domain, wherein the VH domain contains the variable region of sequence >6 shown in FIG. 136A and the antibody is specific for IgE. In some embodiments, antibodies are provided having a VH domain and a VL domain, wherein the VH domain contains the variable region of sequence >7 shown in FIG. 136A and the antibody is specific for VEGF. In some embodiments, antibodies are provided having a VH domain and a VL domain, wherein the VH domain contains the variable region of sequence >8 shown in FIG. 136A and the antibody is specific for IgE. In some embodiments, antibodies are provided having a VH domain and a VL domain, wherein the VH domain contains the variable region of sequence >9 shown in FIG. 136A and the antibody is specific for TNF. In some embodiments, antibodies are provided having a VH domain and a VL domain, wherein the VH domain contains the variable region of sequence >10 shown in FIG. 136A and the antibody is specific for EGFR. In some embodiments, antibodies are provided having a VH domain and a VL domain, wherein the VH domain contains the variable region of sequence >11 shown in FIG. 136A and the antibody is specific for EGFR. In some embodiments, antibodies are provided having a VH domain and a VL domain, wherein the VH domain contains the variable region of sequence >12 shown in FIG. 136A and the antibody is specific for EGFR. In some embodiments, antibodies are provided having a VH domain and a VL domain, wherein the VH domain contains the variable region of sequence >13 shown in FIG. 136A and the antibody is specific for EGFR. In some embodiments, antibodies are provided having a VH domain and a VL domain, wherein the VH domain contains the variable region of sequence >14 shown in FIG. 136A and the antibody is specific for CD20. In some embodiments, antibodies are provided having a VH domain and a VL domain, wherein the VH domain contains the variable region of sequence >15 shown in FIG. 136A and the antibody is specific for CD20. In some embodiments, antibodies are provided having a VH domain and a VL domain, wherein the VH domain contains the variable region of sequence >16 shown in FIG. 136A and the antibody is specific for CD20.

In other embodiments, the present invention provides antibodies having a VH domain and a VL domain, wherein the VL domain contains the variable region of a sequence selected from the sixteen sequences (>1 to >16) shown in FIG. 136B. In some embodiments, antibodies are provided having a VH domain and a VL domain, wherein the VL domain contains the variable region of sequence >1 shown in FIG. 136B and the antibody is specific for CD30. In some embodiments, antibodies are provided having a VH domain and a VL domain, wherein the VL domain contains the variable region of sequence >2 shown in FIG. 136B and the antibody is specific for CD19. In some embodiments, antibodies are provided having a VH domain and a VL domain, wherein the VL domain contains the variable region of sequence >3 shown in FIG. 136B and the antibody is specific for CD40. In some embodiments, antibodies are provided having a VH domain and a VL domain, wherein the VL domain contains the variable region of sequence >4 shown in FIG. 136B and the antibody is specific for HM1.24. In some embodiments, antibodies are provided having a VH domain and a VL domain, wherein the VL domain contains the variable region of sequence >5 shown in FIG. 136B and the antibody is specific for CD19. In some embodiments, antibodies are provided having a VH domain and a VL domain, wherein the VL domain contains the variable region of sequence >6 shown in FIG. 136B and the antibody is specific for IgE. In some embodiments, antibodies are provided having a VH domain and a VL domain, wherein the VL domain contains the variable region of sequence >7 shown in FIG. 136B and the antibody is specific for VEGF. In some embodiments, antibodies are provided having a VH domain and a VL domain, wherein the VL domain contains the variable region of sequence >8 shown in FIG. 136B and the antibody is specific for IgE. In some embodiments, antibodies are provided having a VH domain and a VL domain, wherein the VL domain contains the variable region of sequence >9 shown in FIG. 136B and the antibody is specific for TNF. In some embodiments, antibodies are provided having a VH domain and a VL domain, wherein the VL domain contains the variable region of sequence >10 shown in FIG. 136B and the antibody is specific for EGFR. In some embodiments, antibodies are provided having a VH domain and a VL domain, wherein the VL domain contains the variable region of sequence >11 shown in FIG. 136B and the antibody is specific for EGFR. In some embodiments, antibodies are provided having a VH domain and a VL domain, wherein the VL domain contains the variable region of sequence >12 shown in FIG. 136B and the antibody is specific for EGFR. In some embodiments, antibodies are provided having a VH domain and a VL domain, wherein the VL domain contains the variable region of sequence >13 shown in FIG. 136B and the antibody is specific for EGFR. In some embodiments, antibodies are provided having a VH domain and a VL domain, wherein the VL domain contains the variable region of sequence >14 shown in FIG. 136B and the antibody is specific for CD20. In some embodiments, antibodies are provided having a VH domain and a VL domain, wherein the VL domain contains the variable region of sequence >15 shown in FIG. 136B and the antibody is specific for CD20. In some embodiments, antibodies are provided having a VH domain and a VL domain, wherein the VL domain contains the variable region of sequence >16 shown in FIG. 136B and the antibody is specific for CD20.

In still other embodiments, the present invention provides antibodies having a VH domain and a VL domain, wherein the VH domain contains the variable region of sequence >1 of FIG. 136A and the VL domain contains the variable region of a sequence selected from sequences >1, >2, >3, >4, >5, >6, >7, >8, >9, >10, >11, >12, >13, >14, >15, and >16 of FIG. 136B. In still other embodiments, the present invention provides antibodies having a VH domain and a VL domain, wherein the VH domain contains the variable region of sequence >2 of FIG. 136A and the VL domain contains the variable region of a sequence selected from sequences >1, >2, >3, >4, >5, >6, >7, >8, >9, >10, >11, >12, >13, >14, >15, and >16 of FIG. 136B. In still other embodiments, the present invention provides antibodies having a VH domain and a VL domain, wherein the VH domain contains the variable region of sequence >3 of FIG. 136A and the VL domain contains the variable region of a sequence selected from sequences >1, >2, >3, >4, >5, >6, >7, >8, >9, >10, >11, >12, >13, >14, >15, and >16 of FIG. 136B. In still other embodiments, the present invention provides antibodies having a VH domain and a VL domain, wherein the VH domain contains the variable region of sequence >4 of FIG. 136A and the VL domain contains the variable region of a sequence selected from sequences >1, >2, >3, >4, >5, >6, >7, >8, >9, >10, >11, >12, >13, >14, >15, and >16 of FIG. 136B. In still other embodiments, the present invention provides antibodies having a VH domain and a VL domain, wherein the VH domain contains the variable region of sequence >5 of FIG. 136A and the VL domain contains the variable region of a sequence selected from sequences >1, >2, >3, >4, >5, >6, >7, >8, >9, >10, >11, >12, >13, >14, >15, and >16 of FIG. 136B. In still other embodiments, the present invention provides antibodies having a VH domain and a VL domain, wherein the VH domain contains the variable region of sequence >6 of FIG. 136A and the VL domain contains the variable region of a sequence selected from sequences >1, >2, >3, >4, >5, >6, >7, >8, >9, >10, >11, >12, >13, >14, >15, and >16 of FIG. 136B. In still other embodiments, the present invention provides antibodies having a VH domain and a VL domain, wherein the VH domain contains the variable region of sequence >7 of FIG. 136A and the VL domain contains the variable region of a sequence selected from sequences >1, >2, >3, >4, >5, >6, >7, >8, >9, >10, >11, >12, >13, >14, >15, and >16 of FIG. 136B. In still other embodiments, the present invention provides antibodies having a VH domain and a VL domain, wherein the VH domain contains the variable region of sequence >8 of FIG. 136A and the VL domain contains the variable region of a sequence selected from sequences >1, >2, >3, >4, >5, >6, >7, >8, >9, >10, >11, >12, >13, >14, >15, and >16 of FIG. 136B. In still other embodiments, the present invention provides antibodies having a VH domain and a VL domain, wherein the VH domain contains the variable region of sequence >9 of FIG. 136A and the VL domain contains the variable region of a sequence selected from sequences >1, >2, >3, >4, >5, >6, >7, >8, >9, >10, >11, >12, >13, >14, >15, and >16 of FIG. 136B. In still other embodiments, the present invention provides antibodies having a VH domain and a VL domain, wherein the VH domain contains the variable region of sequence >10 of FIG. 136A and the VL domain contains the variable region of a sequence selected from sequences >1, >2, >3, >4, >5, >6, >7, >8, >9, >10, >11, >12, >13, >14, >15, and >16 of FIG. 136B. In still other embodiments, the present invention provides antibodies having a VH domain and a VL domain, wherein the VH domain contains the variable region of sequence >11 of FIG. 136A and the VL domain contains the variable region of a sequence selected from sequences >1, >2, >3, >4, >5, >6, >7, >8, >9, >10, >11, >12, >13, >14, >15, and >16 of FIG. 136B. In still other embodiments, the present invention provides antibodies having a VH domain and a VL domain, wherein the VH domain contains the variable region of sequence >12 of FIG. 136A and the VL domain contains the variable region of a sequence selected from sequences >1, >2, >3, >4, >5, >6, >7, >8, >9, >10, >11, >12, >13, >14, >15, and >16 of FIG. 136B. In still other embodiments, the present invention provides antibodies having a VH domain and a VL domain, wherein the VH domain contains the variable region of sequence >13 of FIG. 136A and the VL domain contains the variable region of a sequence selected from sequences >1, >2, >3, >4, >5, >6, >7, >8, >9, >10, >11, >12, >13, >14, >15, and >16 of FIG. 136B. In still other embodiments, the present invention provides antibodies having a VH domain and a VL domain, wherein the VH domain contains the variable region of sequence >14 of FIG. 136A and the VL domain contains the variable region of a sequence selected from sequences >1, >2, >3, >4, >5, >6, >7, >8, >9, >10, >11, >12, >13, >14, >15, and >16 of FIG. 136B. In still other embodiments, the present invention provides antibodies having a VH domain and a VL domain, wherein the VH domain contains the variable region of sequence >15 of FIG. 136A and the VL domain contains the variable region of a sequence selected from sequences >1, >2, >3, >4, >5, >6, >7, >8, >9, >10, >11, >12, >13, >14, >15, and >16 of FIG. 136B. In still other embodiments, the present invention provides antibodies having a VH domain and a VL domain, wherein the VH domain contains the variable region of sequence >16 of FIG. 136A and the VL domain contains the variable region of a sequence selected from sequences >1, >2, >3, >4, >5, >6, >7, >8, >9, >10, >11, >12, >13, >14, >15, and >16 of FIG. 136B.

Each VH and VL is composed of three hypervariable regions (“complementary determining regions,” “CDRs”) and four FRs, arranged from amino-terminus to carboxy-terminus in the following order: FR1-CDR1-FR2-CDR2-FR3-CDR3-FR4.

The hypervariable region generally encompasses amino acid residues from about amino acid residues 24-34 (LCDR1; “L” denotes light chain), 50-56 (LCDR2) and 89-97 (LCDR3) in the light chain variable region and around about 31-35B (HCDR1; “H” denotes heavy chain), 50-65 (HCDR2), and 95-102 (HCDR3) in the heavy chain variable region; Kabat et al., SEQUENCES OF PROTEINS OF IMMUNOLOGICAL INTEREST, 5th Ed. Public Health Service, National Institutes of Health, Bethesda, Md. (1991) and/or those residues forming a hypervariable loop (e.g., residues 26-32 (LCDR1), 50-52 (LCDR2) and 91-96 (LCDR3) in the light chain variable region and 26-32 (HCDR1), 53-55 (HCDR2) and 96-101 (HCDR3) in the heavy chain variable region; Chothia and Lesk (1987) J. Mol. Biol. 196:901-917. Specific CDRs of the invention are described below.

In some embodiments, the present invention provides antibodies having a VH domain and a VL domain, wherein the VH domain contains the HCDR1 (VH CDR1), HCDR2 (VH CDR2), and HCDR3 (VH CDR3) of a sequence selected from the sixteen sequences (>1 to >16) shown in FIG. 136A. In other embodiments, the present invention provides antibodies having a VH domain and a VL domain, wherein the VL domain contains the LCDR1 (VL CDR1), LCDR2 (VL CDR2), and LCDR3 (VL CDR3) of a sequence selected from the sixteen sequences (>1 to >16) shown in FIG. 136B.

In some embodiments, the present invention provides antibodies having a VH domain and a VL domain, wherein the VH domain contains the HCDR1 (VH CDR1), HCDR2 (VH CDR2), and HCDR3 (VH CDR3) of sequence >1 of FIG. 136A and the VL domain contains the LCDR1 (VL CDR1), LCDR2 (VL CDR2), and LCDR3 (VL CDR3) of sequence >1 of FIG. 136B, wherein the antibody is specific for CD30.

In some embodiments, the present invention provides antibodies having a VH domain and a VL domain, wherein the VH domain contains the HCDR1 (VH CDR1), HCDR2 (VH CDR2), and HCDR3 (VH CDR3) of sequence >2 of FIG. 136A and the VL domain contains the LCDR1 (VL CDR1), LCDR2 (VL CDR2), and LCDR3 (VL CDR3) of sequence >2 of FIG. 136B, wherein the antibody is specific for CD19.

In some embodiments, the present invention provides antibodies having a VH domain and a VL domain, wherein the VH domain contains the HCDR1 (VH CDR1), HCDR2 (VH CDR2), and HCDR3 (VH CDR3) of sequence >3 of FIG. 136A and the VL domain contains the LCDR1 (VL CDR1), LCDR2 (VL CDR2), and LCDR3 (VL CDR3) of sequence >3 of FIG. 136B, wherein the antibody is specific for CD40.

In some embodiments, the present invention provides antibodies having a VH domain and a VL domain, wherein the VH domain contains the HCDR1 (VH CDR1), HCDR2 (VH CDR2), and HCDR3 (VH CDR3) of sequence >4 of FIG. 136A and the VL domain contains the LCDR1 (VL CDR1), LCDR2 (VL CDR2), and LCDR3 (VL CDR3) of sequence >4 of FIG. 136B, wherein the antibody is specific for HM1.24.

In some embodiments, the present invention provides antibodies having a VH domain and a VL domain, wherein the VH domain contains the HCDR1 (VH CDR1), HCDR2 (VH CDR2), and HCDR3 (VH CDR3) of sequence >5 of FIG. 136A and the VL domain contains the LCDR1 (VL CDR1), LCDR2 (VL CDR2), and LCDR3 (VL CDR3) of sequence >5 of FIG. 136B, wherein the antibody is specific for CD19.

In some embodiments, the present invention provides antibodies having a VH domain and a VL domain, wherein the VH domain contains the HCDR1 (VH CDR1), HCDR2 (VH CDR2), and HCDR3 (VH CDR3) of sequence >6 of FIG. 136A and the VL domain contains the LCDR1 (VL CDR1), LCDR2 (VL CDR2), and LCDR3 (VL CDR3) of sequence >6 of FIG. 136B, wherein the antibody is specific for IgE.

In some embodiments, the present invention provides antibodies having a VH domain and a VL domain, wherein the VH domain contains the HCDR1 (VH CDR1), HCDR2 (VH CDR2), and HCDR3 (VH CDR3) of sequence >7 of FIG. 136A and the VL domain contains the LCDR1 (VL CDR1), LCDR2 (VL CDR2), and LCDR3 (VL CDR3) of sequence >7 of FIG. 136B, wherein the antibody is specific for VEGF.

In some embodiments, the present invention provides antibodies having a VH domain and a VL domain, wherein the VH domain contains the HCDR1 (VH CDR1), HCDR2 (VH CDR2), and HCDR3 (VH CDR3) of sequence >8 of FIG. 136A and the VL domain contains the LCDR1 (VL CDR1), LCDR2 (VL CDR2), and LCDR3 (VL CDR3) of sequence >8 of FIG. 136B, wherein the antibody is specific for IgE.

In some embodiments, the present invention provides antibodies having a VH domain and a VL domain, wherein the VH domain contains the HCDR1 (VH CDR1), HCDR2 (VH CDR2), and HCDR3 (VH CDR3) of sequence >9 of FIG. 136A and the VL domain contains the LCDR1 (VL CDR1), LCDR2 (VL CDR2), and LCDR3 (VL CDR3) of sequence >9 of FIG. 136B, wherein the antibody is specific for TNF.

In some embodiments, the present invention provides antibodies having a VH domain and a VL domain, wherein the VH domain contains the HCDR1 (VH CDR1), HCDR2 (VH CDR2), and HCDR3 (VH CDR3) of sequence >10 of FIG. 136A and the VL domain contains the LCDR1 (VL CDR1), LCDR2 (VL CDR2), and LCDR3 (VL CDR3) of sequence >10 of FIG. 136B, wherein the antibody is specific for EGFR.

In some embodiments, the present invention provides antibodies having a VH domain and a VL domain, wherein the VH domain contains the HCDR1 (VH CDR1), HCDR2 (VH CDR2), and HCDR3 (VH CDR3) of sequence >11 of FIG. 136A and the VL domain contains the LCDR1 (VL CDR1), LCDR2 (VL CDR2), and LCDR3 (VL CDR3) of sequence >11 of FIG. 136B, wherein the antibody is specific for EGFR.

In some embodiments, the present invention provides antibodies having a VH domain and a VL domain, wherein the VH domain contains the HCDR1 (VH CDR1), HCDR2 (VH CDR2), and HCDR3 (VH CDR3) of sequence >12 of FIG. 136A and the VL domain contains the LCDR1 (VL CDR1), LCDR2 (VL CDR2), and LCDR3 (VL CDR3) of sequence >12 of FIG. 136B, wherein the antibody is specific for EGFR.

In some embodiments, the present invention provides antibodies having a VH domain and a VL domain, wherein the VH domain contains the HCDR1 (VH CDR1), HCDR2 (VH CDR2), and HCDR3 (VH CDR3) of sequence >13 of FIG. 136A and the VL domain contains the LCDR1 (VL CDR1), LCDR2 (VL CDR2), and LCDR3 (VL CDR3) of sequence >13 of FIG. 136B, wherein the antibody is specific for EGFR.

In some embodiments, the present invention provides antibodies having a VH domain and a VL domain, wherein the VH domain contains the HCDR1 (VH CDR1), HCDR2 (VH CDR2), and HCDR3 (VH CDR3) of sequence >14 of FIG. 136A and the VL domain contains the LCDR1 (VL CDR1), LCDR2 (VL CDR2), and LCDR3 (VL CDR3) of sequence >14 of FIG. 136B, wherein the antibody is specific for CD20.

In some embodiments, the present invention provides antibodies having a VH domain and a VL domain, wherein the VH domain contains the HCDR1 (VH CDR1), HCDR2 (VH CDR2), and HCDR3 (VH CDR3) of sequence >15 of FIG. 136A and the VL domain contains the LCDR1 (VL CDR1), LCDR2 (VL CDR2), and LCDR3 (VL CDR3) of sequence >15 of FIG. 136B, wherein the antibody is specific for CD20.

In some embodiments, the present invention provides antibodies having a VH domain and a VL domain, wherein the VH domain contains the HCDR1 (VH CDR1), HCDR2 (VH CDR2), and HCDR3 (VH CDR3) of sequence >16 of FIG. 136A and the VL domain contains the LCDR1 (VL CDR1), LCDR2 (VL CDR2), and LCDR3 (VL CDR3) of sequence >16 of FIG. 136B, wherein the antibody is specific for CD20.

In still other embodiments, the present invention provides antibodies having a VH domain and a VL domain, wherein the VH domain contains the HCDR1 (VH CDR1), HCDR2 (VH CDR2), and HCDR3 (VH CDR3) of sequence >1 of FIG. 136A and the VL domain contains the LCDR1 (VL CDR1), LCDR2 (VL CDR2), and LCDR3 (VL CDR3) of a sequence selected from sequences >1, >2, >3, >4, >5, >6, >7, >8, >9, >10, >11, >12, >13, >14, >15, and >16 of FIG. 136B. In still other embodiments, the present invention provides antibodies having a VH domain and a VL domain, wherein the VH domain contains the HCDR1 (VH CDR1), HCDR2 (VH CDR2), and HCDR3 (VH CDR3) of sequence >2 of FIG. 136A and the VL domain contains the LCDR1 (VL CDR1), LCDR2 (VL CDR2), and LCDR3 (VL CDR3) of a sequence selected from sequences >1, >2, >3, >4, >5, >6, >7, >8, >9, >10, >11, >12, >13, >14, >15, and >16 of FIG. 136B. In still other embodiments, the present invention provides antibodies having a VH domain and a VL domain, wherein the VH domain contains the HCDR1 (VH CDR1), HCDR2 (VH CDR2), and HCDR3 (VH CDR3) of sequence >3 of FIG. 136A and the VL domain contains the LCDR1 (VL CDR1), LCDR2 (VL CDR2), and LCDR3 (VL CDR3) of a sequence selected from sequences >1, >2, >3, >4, >5, >6, >7, >8, >9, >10, >11, >12, >13, >14, >15, and >16 of FIG. 136B. In still other embodiments, the present invention provides antibodies having a VH domain and a VL domain, wherein the VH domain contains the HCDR1 (VH CDR1), HCDR2 (VH CDR2), and HCDR3 (VH CDR3) of sequence >4 of FIG. 136A and the VL domain contains the LCDR1 (VL CDR1), LCDR2 (VL CDR2), and LCDR3 (VL CDR3) of a sequence selected from sequences >1, >2, >3, >4, >5, >6, >7, >8, >9, >10, >11, >12, >13, >14, >15, and >16 of FIG. 136B. In still other embodiments, the present invention provides antibodies having a VH domain and a VL domain, wherein the VH domain contains the HCDR1 (VH CDR1), HCDR2 (VH CDR2), and HCDR3 (VH CDR3) of sequence >5 of FIG. 136A and the VL domain contains the LCDR1 (VL CDR1), LCDR2 (VL CDR2), and LCDR3 (VL CDR3) of a sequence selected from sequences >1, >2, >3, >4, >5, >6, >7, >8, >9, >10, >11, >12, >13, >14, >15, and >16 of FIG. 136B. In still other embodiments, the present invention provides antibodies having a VH domain and a VL domain, wherein the VH domain contains the HCDR1 (VH CDR1), HCDR2 (VH CDR2), and HCDR3 (VH CDR3) of sequence >6 of FIG. 136A and the VL domain contains the LCDR1 (VL CDR1), LCDR2 (VL CDR2), and LCDR3 (VL CDR3) of a sequence selected from sequences >1, >2, >3, >4, >5, >6, >7, >8, >9, >10, >11, >12, >13, >14, >15, and >16 of FIG. 136B. In still other embodiments, the present invention provides antibodies having a VH domain and a VL domain, wherein the VH domain contains the HCDR1 (VH CDR1), HCDR2 (VH CDR2), and HCDR3 (VH CDR3) of sequence >7 of FIG. 136A and the VL domain contains the LCDR1 (VL CDR1), LCDR2 (VL CDR2), and LCDR3 (VL CDR3) of a sequence selected from sequences >1, >2, >3, >4, >5, >6, >7, >8, >9, >10, >11, >12, >13, >14, >15, and >16 of FIG. 136B. In still other embodiments, the present invention provides antibodies having a VH domain and a VL domain, wherein the VH domain contains the HCDR1 (VH CDR1), HCDR2 (VH CDR2), and HCDR3 (VH CDR3) of sequence >8 of FIG. 136A and the VL domain contains the LCDR1 (VL CDR1), LCDR2 (VL CDR2), and LCDR3 (VL CDR3) of a sequence selected from sequences >1, >2, >3, >4, >5, >6, >7, >8, >9, >10, >11, >12, >13, >14, >15, and >16 of FIG. 136B. In still other embodiments, the present invention provides antibodies having a VH domain and a VL domain, wherein the VH domain contains the HCDR1 (VH CDR1), HCDR2 (VH CDR2), and HCDR3 (VH CDR3) of sequence >9 of FIG. 136A and the VL domain contains the LCDR1 (VL CDR1), LCDR2 (VL CDR2), and LCDR3 (VL CDR3) of a sequence selected from sequences >1, >2, >3, >4, >5, >6, >7, >8, >9, >10, >11, >12, >13, >14, >15, and >16 of FIG. 136B. In still other embodiments, the present invention provides antibodies having a VH domain and a VL domain, wherein the VH domain contains the HCDR1 (VH CDR1), HCDR2 (VH CDR2), and HCDR3 (VH CDR3) of sequence >10 of FIG. 136A and the VL domain contains the LCDR1 (VL CDR1), LCDR2 (VL CDR2), and LCDR3 (VL CDR3) of a sequence selected from sequences >1, >2, >3, >4, >5, >6, >7, >8, >9, >10, >11, >12, >13, >14, >15, and >16 of FIG. 136B. In still other embodiments, the present invention provides antibodies having a VH domain and a VL domain, wherein the VH domain contains the HCDR1 (VH CDR1), HCDR2 (VH CDR2), and HCDR3 (VH CDR3) of sequence >11 of FIG. 136A and the VL domain contains the LCDR1 (VL CDR1), LCDR2 (VL CDR2), and LCDR3 (VL CDR3) of a sequence selected from sequences >1, >2, >3, >4, >5, >6, >7, >8, >9, >10, >11, >12, >13, >14, >15, and >16 of FIG. 136B. In still other embodiments, the present invention provides antibodies having a VH domain and a VL domain, wherein the VH domain contains the HCDR1 (VH CDR1), HCDR2 (VH CDR2), and HCDR3 (VH CDR3) of sequence >12 of FIG. 136A and the VL domain contains the LCDR1 (VL CDR1), LCDR2 (VL CDR2), and LCDR3 (VL CDR3) of a sequence selected from sequences >1, >2, >3, >4, >5, >6, >7, >8, >9, >10, >11, >12, >13, >14, >15, and >16 of FIG. 136B. In still other embodiments, the present invention provides antibodies having a VH domain and a VL domain, wherein the VH domain contains the HCDR1 (VH CDR1), HCDR2 (VH CDR2), and HCDR3 (VH CDR3) of sequence >13 of FIG. 136A and the VL domain contains the LCDR1 (VL CDR1), LCDR2 (VL CDR2), and LCDR3 (VL CDR3) of a sequence selected from sequences >1, >2, >3, >4, >5, >6, >7, >8, >9, >10, >11, >12, >13, >14, >15, and >16 of FIG. 136B. In still other embodiments, the present invention provides antibodies having a VH domain and a VL domain, wherein the VH domain contains the HCDR1 (VH CDR1), HCDR2 (VH CDR2), and HCDR3 (VH CDR3) of sequence >14 of FIG. 136A and the VL domain contains the LCDR1 (VL CDR1), LCDR2 (VL CDR2), and LCDR3 (VL CDR3) of a sequence selected from sequences >1, >2, >3, >4, >5, >6, >7, >8, >9, >10, >11, >12, >13, >14, >15, and >16 of FIG. 136B. In still other embodiments, the present invention provides antibodies having a VH domain and a VL domain, wherein the VH domain contains the HCDR1 (VH CDR1), HCDR2 (VH CDR2), and HCDR3 (VH CDR3) of sequence >15 of FIG. 136a and the VL domain contains the LCDR1 (VL CDR1), LCDR2 (VL CDR2), and LCDR3 (VL CDR3) of a sequence selected from sequences >1, >2, >3, >4, >5, >6, >7, >8, >9, >10, >11, >12, >13, >14, >15, and >16 of FIG. 136b. In still other embodiments, the present invention provides antibodies having a VH domain and a VL domain, wherein the VH domain contains the HCDR1 (VH CDR1), HCDR2 (VH CDR2), and HCDR3 (VH CDR3) of sequence >16 of FIG. 136a and the VL domain contains the LCDR1 (VL CDR1), LCDR2 (VL CDR2), and LCDR3 (VL CDR3) of a sequence selected from sequences >1, >2, >3, >4, >5, >6, >7, >8, >9, >10, >11, >12, >13, >14, >15, and >16 of FIG. 136b.

Throughout the present specification, the Kabat numbering system is generally used when referring to a residue in the variable domain (approximately, residues 1-107 of the light chain variable region and residues 1-113 of the heavy chain variable region) (e.g, Kabat et al., supra (1991)).

The CDRs contribute to the formation of the antigen-binding, or more specifically, epitope binding site of antibodies. “Epitope” refers to a determinant that interacts with a specific antigen binding site in the variable region of an antibody molecule known as a paratope. Epitopes are groupings of molecules such as amino acids or sugar side chains and usually have specific structural characteristics, as well as specific charge characteristics. A single antigen may have more than one epitope.

The epitope may comprise amino acid residues directly involved in the binding (also called immunodominant component of the epitope) and other amino acid residues, which are not directly involved in the binding, such as amino acid residues which are effectively blocked by the specifically antigen binding peptide; in other words, the amino acid residue is within the footprint of the specifically antigen binding peptide.

Epitopes may be either conformational or linear. A conformational epitope is produced by spatially juxtaposed amino acids from different segments of the linear polypeptide chain. A linear epitope is one produced by adjacent amino acid residues in a polypeptide chain. Conformational and nonconformational epitopes may be distinguished in that the binding to the former but not the latter is lost in the presence of denaturing solvents.

An epitope typically includes at least 3, and more usually, at least 5 or 8-10 amino acids in a unique spatial conformation. Antibodies that recognize the same epitope can be verified in a simple immunoassay showing the ability of one antibody to block the binding of another antibody to a target antigen, for example “binning.”

In some embodiments, the antibodies are full length. By “full length antibody” herein is meant the structure that constitutes the natural biological form of an antibody, including variable and constant regions, including one or more modifications as outlined herein.

Alternatively, the antibodies can be a variety of structures, including, but not limited to, antibody fragments, monoclonal antibodies, minibodies, domain antibodies, synthetic antibodies (sometimes referred to herein as “antibody mimetics”), chimeric antibodies, humanized antibodies, antibody fusions (sometimes referred to as “antibody conjugates”), and fragments of each, respectively.

Antibody Fragments

In one embodiment, the antibody is an antibody fragment. Of particular interest are antibodies that comprise Fc regions, Fc fusions, and the constant region of the heavy chain (CH1-hinge-CH2-CH3) or constant heavy region fusions.

Specific antibody fragments include, but are not limited to, (i) the Fab fragment consisting of VL, VH, CL and CH1 domains, (ii) the Fd fragment consisting of the VH and CH1 domains, (iii) the Fv fragment consisting of the VL and VH domains of a single antibody; (iv) the dAb fragment (Ward et al., 1989, Nature 341:544-546, entirely incorporated by reference) which consists of a single variable, (v) isolated CDR regions, (vi) F(ab′)2 fragments, a bivalent fragment comprising two linked Fab fragments (vii) single chain Fv molecules (scFv), wherein a VH domain and a VL domain are linked by a peptide linker which allows the two domains to associate to form an antigen binding site (Bird et al., 1988, Science 242:423-426, Huston et al., 1988, Proc. Natl. Acad. Sci. U.S.A. 85:5879-5883, entirely incorporated by reference), all entirely incorporated by reference). The antibody fragments may be modified. For example, the molecules may be stabilized by the incorporation of disulphide bridges linking the VH and VL domains (Reiter et al., 1996, Nature Biotech. 14:1239-1245, entirely incorporated by reference).

### Chimeric and Humanized Antibodies

In some embodiments, the scaffold components can be a mixture from different species. As such, if the protein is an antibody, such antibody may be a chimeric antibody and/or a humanized antibody. In general, both “chimeric antibodies” and “humanized antibodies” refer to antibodies that combine regions from more than one species. For example, “chimeric antibodies” traditionally comprise variable region(s) from a mouse (or rat, in some cases) and the constant region(s) from a human. “Humanized antibodies” generally refer to non-human antibodies that have had the variable-domain framework regions swapped for sequences found in human antibodies. Generally, in a humanized antibody, the entire antibody, except the CDRs, is encoded by a polynucleotide of human origin or is identical to such an antibody except within its CDRs. The CDRs, some or all of which are encoded by nucleic acids originating in a non-human organism, are grafted into the beta-sheet framework of a human antibody variable region to create an antibody, the specificity of which is determined by the engrafted CDRs. The creation of such antibodies is described in, e.g., WO 92/11018, Jones, 1986, Nature 321:522-525, Verhoeyen et al., 1988, Science 239:1534-1536, all entirely incorporated by reference. “Backmutation” of selected acceptor framework residues to the corresponding donor residues is often required to regain affinity that is lost in the initial grafted construct (U.S. Pat. No. 5,530,101; U.S. Pat. No. 5,585,089; U.S. Pat. No. 5,693,761; U.S. Pat. No. 5,693,762; U.S. Pat. No. 6,180,370; U.S. Pat. No. 5,859,205; U.S. Pat. No. 5,821,337; U.S. Pat. No. 6,054,297; U.S. Pat. No. 6,407,213, all entirely incorporated by reference). The humanized antibody optimally also will comprise at least a portion of an immunoglobulin constant region, typically that of a human immunoglobulin, and thus will typically comprise a human Fc region. Humanized antibodies can also be generated using mice with a genetically engineered immune system. Roque et al., 2004, Biotechnol. Prog. 20:639-654, entirely incorporated by reference. A variety of techniques and methods for humanizing and reshaping non-human antibodies are well known in the art (See Tsurushita & Vasquez, 2004, Humanization of Monoclonal Antibodies, Molecular Biology of B Cells, 533-545, Elsevier Science (USA), and references cited therein, all entirely incorporated by reference). Humanization methods include but are not limited to methods described in Jones et al., 1986, Nature 321:522-525; Riechmann et al., 1988; Nature 332:323-329; Verhoeyen et al., 1988, Science, 239:1534-1536; Queen et al., 1989, Proc Natl Acad Sci, USA 86:10029-33; He et al., 1998, J. Immunol. 160: 1029-1035; Carter et al., 1992, Proc Natl Acad Sci USA 89:4285-9, Presta et al., 1997, Cancer Res. 57(20):4593-9; Gorman et al., 199I, Proc. Natl. Acad. Sci. USA 88:4181-4185; O'Connor et al., 1998, Protein Eng 11:321-8, all entirely incorporated by reference. Humanization or other methods of reducing the immunogenicity of nonhuman antibody variable regions may include resurfacing methods, as described for example in Roguska et al., 1994, Proc. Natl. Acad. Sci. USA 91:969-973, entirely incorporated by reference. In one embodiment, the parent antibody has been affinity matured, as is known in the art. Structure-based methods may be employed for humanization and affinity maturation, for example as described in U.S. Ser. No. 11/004,590. Selection based methods may be employed to humanize and/or affinity mature antibody variable regions, including but not limited to methods described in Wu et al., 1999, J. Mol. Biol. 294:151-162; Baca et al., 1997, J. Biol. Chem. 272(16):10678-10684; Rosok et al., 1996, J. Biol. Chem. 271(37): 22611-22618; Rader et al., 1998, Proc. Natl. Acad. Sci. USA 95: 8910-8915; Krauss et al., 2003, Protein Engineering 16(10):753-759, all entirely incorporated by reference. Other humanization methods may involve the grafting of only parts of the CDRs, including but not limited to methods described in U.S. Ser. No. 09/810,510; Tan et al., 2002, J. Immunol. 169:1119-1125; De Pascalis et al., 2002, J. Immunol. 169:3076-3084, all entirely incorporated by reference.

In one embodiment, the parent Fc polypeptide to be modified is a fully human antibody. Fully human antibodies may be obtained, for example, using transgenic mice (Bruggemann et al., 1997, Curr Opin Biotechnol 8:455-458) or human antibody libraries coupled with selection methods (Griffiths et al., 1998, Curr Opin Biotechnol 9:102-108).

### Bispecific Antibodies

In one embodiment, the antibodies of the invention multispecific antibody, and notably a bispecific antibody, also sometimes referred to as “diabodies”. These are antibodies that bind to two (or more) different antigens. Diabodies can be manufactured in a variety of ways known in the art (Holliger and Winter, 1993, Current Opinion Biotechnol. 4:446-449), e.g., prepared chemically or from hybrid hybridomas.

### Minibodies

In one embodiment, the antibody is a minibody. Minibodies are minimized antibody-like proteins comprising a scFv joined to a CH3 domain. Hu et al., 1996, Cancer Res. 56:3055-3061, entirely incorporated by reference. In some cases, the scFv can be joined to the Fc region, and may include some or the entire hinge region.

### Fc Fusion Proteins

In addition to antibody constructs, the invention further provides Fc fusion proteins. That is, rather than have the Fc domain of an antibody joined to an antibody variable region, the Fc domain can be joined to other moieties, particularly binding moieties such as ligands. By “Fc fusion” as used herein is meant a protein wherein one or more polypeptides is operably linked to an Fc region. Fc fusion is herein meant to be synonymous with the terms “immunoadhesin”, “Ig fusion”, “Ig chimera”, and “receptor globulin” (sometimes with dashes) as used in the prior art (Chamow et al., 1996, Trends Biotechnol 14:52-60; Ashkenazi et al., 1997, Curr Opin Immunol 9:195-200, both entirely incorporated by reference). An Fc fusion combines the Fc region of an immunoglobulin with a fusion partner, which in general can be any protein or small molecule. Virtually any protein or small molecule may be linked to Fc to generate an Fc fusion. Protein fusion partners may include, but are not limited to, the variable region of any antibody, the target-binding region of a receptor, an adhesion molecule, a ligand, an enzyme, a cytokine, a chemokine, or some other protein or protein domain. Small molecule fusion partners may include any therapeutic agent that directs the Fc fusion to a therapeutic target. Such targets may be any molecule, preferably an extracellular receptor, which is implicated in disease. Thus, the IgG variants can be linked to one or more fusion partners.

Thus, while many embodiments herein depict antibody components such as variable heavy and light chains or scFvs, other binding moeities can be fused to Fc regions to form Fc fusion proteins. Suitable receptors and ligands are outlined below in the “Target” section.

### Fc Receptor Binding Properties

The Fc variants of the present invention may be optimized for a variety of Fc receptor binding properties. An Fc variant that is engineered or predicted to display one or more optimized properties is herein referred to as an “optimized Fc variant”. Properties that may be optimized include but are not limited to enhanced or reduced affinity for an FcγR. In a preferred embodiment, the Fc variants of the present invention are optimized to possess enhanced affinity for a human activating FcγR, preferably FcγR1, FcγRIIa, FcγRIIc, FcγRIIIa, and FcγRIIIb, most preferrably FcγRIIa and FcγRIIIa. In an alternately preferred embodiment, the Fc variants are optimized to possess reduced affinity for the human inhibitory receptor FcγRIIb. These preferred embodiments are anticipated to provide Fc polypeptides with enhanced therapeutic properties in humans, for example enhanced effector function and greater anti-cancer potency. In other embodiments, Fc variants of the present invention provide enhanced affinity for one or more FcγRs, yet reduced affinity for one or more other FcγRs. For example, an Fc variant of the present invention may have enhanced binding to FcγR1, FcγRIIa, and/or FcγRIIIa, yet reduced binding to FcγRIIb.

For example, engineered Fc's with enhanced binding to both FcγRI and FcγRIIIa is effective at activating macrophages. Combining these enhanced Fc's with anti-CD20 antibodies, such as Rituxan®, or some of the antibodies disclosed by Uchida et al. (in particular MB20-11 or MB20-18) can create very effective therapies that can be superior at activating macrophages, a key driver of anti-CD20 efficacy. These therapies are also superior for depletion of splenic and other tissue B cells. Improvement of RI or RIII binding alone igreatly improves macrophage activation. Xencor data demonstrating that macrophage phagocytosis of tumor cells is more effective with antibodies that have heightened FcγRI+FcγRIII binding disclosed herein directly demonstrates the effectiveness of these novel Fc's at recruiting macrophages.

The present invention provides a variety of engineering methods, many of which are based on more sophisticated and efficient techniques, which are used develop anti-CD20 antibodies that are optimized for the desired properties. The described engineering methods provide design strategies to guide Fc modification, computational screening methods to design favorable Fc variants, library generation approaches for determining promising variants for experimental investigation, and an array of experimental production and screening methods for determining the Fc variants with favorable properties.

By “greater affinity” or “improved affinity” or “enhanced affinity” or “better affinity” than a parent Fc polypeptide, as used herein is meant that an Fc variant binds to an Fc receptor with a significantly higher equilibrium constant of association (K_{A}) or lower equilibrium constant of dissociation (K_{D}) than the parent Fc polypeptide when the amounts of variant and parent polypeptide in the binding assay are essentially the same. For example, the Fc variant with improved Fc receptor binding affinity may display from about 5 fold to about 1000 fold, e.g., from about 10 fold to about 500 fold improvement in Fc receptor binding affinity compared to the parent Fc polypeptide, where Fc receptor binding affinity is determined, for example, as disclosed in the Examples herein. Accordingly, by “reduced affinity” as compared to a parent Fc polypeptide as used herein is meant that an Fc variant binds an Fc receptor with significantly lower K_{A} or higher K_{D} than the parent Fc polypeptide.

In a preferred embodiment of the invention, the Fc variants provide selectively enhanced affinity to one or more human activating receptors relative to the inhibitory receptor FcγRIIb. Selectively enhanced affinity to an activating receptor relative to FcγRIIb means either that the Fc variant has improved affinity for the activating receptor as compared to the parent Fc polypeptide but has reduced affinity for FcγRIIb as compared to the parent Fc polypeptide, or it means that the Fc variant has improved affinity for both activating and inhibitory receptors as compared to the parent Fc polypeptide, however the improvement in affinity is greater for the activating receptor than it is for FcγRIIb. The purpose of grouping both of these Fc receptor properties together is that currently it is not known for cells that express both activating and inhibitory receptors whether activation/inhibition is determined by the absolute threshold of FcγRIIb engagement, or by the relative engagement by activating and inhibitory receptors. The preferred application of Fc variants with such Fc receptor affinity profiles is to impart antibodies, Fc fusions, or other Fc polypeptides with enhanced FcγR-mediated effector function and cellular activation, specifically for cells that express both activating and inhibitory receptors including but not limited to neutrophils, monocytes and macrophages, and dendritic cells.

In alternately preferred embodiments of the present invention, the Fc variants reduce or ablate binding to one or more FcγRs, reduce or ablate binding to one or more complement proteins, reduce or ablate one or more FcγR-mediated effector functions, and/or reduce or ablate one or more complement-mediated effector functions. In some embodiments, insertions and/or deletions can be used to ablate the activity, and then amino acid substitutions can be used to increase binding, in many cases to one or more selected FcγRs.

A promising means for enhancing the anti-tumor potency of antibodies is via enhancement of their ability to mediate cytotoxic effector functions such as ADCC, ADCP, and CDC. The importance of FcγR-mediated effector functions for the anti-cancer activity of antibodies has been demonstrated in mice (Clynes et al., 1998, Proc Natl Acad Sci USA 95:652-656; Clynes et al., 2000, Nat Med 6:443-446, both hereby entirely incorporated by reference), and the affinity of interaction between Fc and certain FcγRs correlates with targeted cytotoxicity in cell-based assays (Shields et al., 2001, J Biol Chem 276:6591-6604; Presta et al., 2002, Biochem Soc Trans 30:487-490; Shields et al., 2002, J Biol Chem 277:26733-26740, all hereby entirely incorporated by reference). A critical set of data supporting the relevance of FcγR-mediated effector functions in antibody therapeutic mechanism are the correlations observed between clinical efficacy in humans and their allotype of high and low affinity polymorphic forms of FcγRs. In particular, human IgG1 binds with greater affinity to the V158 isoform of FcγRIIIa than the F158 isoform. This difference in affinity, and its effect FcγR-mediated effector functions such as ADCC and/or ADCP, has been shown to be a significant determinant of the efficacy of the anti-CD20 antibody rituximab (Rituxan®, BiogenIdec). Patients with the V158 allotype respond favorably to rituximab treatment; however, patients with the lower affinity F158 allotype respond poorly (Cartron et al., 2002, Blood 99:754-758; Weng & Levy, 2003, J Clin Oncol, 21(21):3940-3947, hereby entirely incorporated by reference). Approximately 10-20% of humans are V158N158 homozygous, 45% are V158/F158 heterozygous, and 35-45% of humans are F158/F158 homozygous (Lehrnbecher et al., 1999, Blood 94:4220-4232; Cartron et al., 2002, Blood 99:754-758, both hereby entirely incorporated by reference). Thus, 80-90% of humans are poor responders, e.g., they have at least one allele of the F158 FcγRIIIa. Correlations between polymorphisms and clinical outcome have also been documented for the activating receptor FcγRIIa (Weng & Levy, 2003, J Clin Oncol, 21(21):3940-3947; Cheung et al., 2006 J Clin Oncol 24(18):1-6; herein expressly incorporated by reference). The H131 and R131 allotypes of this receptor are approximately equally present in the human population. Non-Hodgkin's lymphoma patients homozygous for the H131 isoform, which binds more tightly to human IgG2 than R131 FcγRIIa, responded better to anti-CD20 rituximab therapy than those homozygous for R131 FcγRIIa (Weng & Levy, 2003, J Clin Oncol, 21(21):3940-3947). The FcγRIIa polymorphism also correlated with clinical outcome following immunotherapy of neuroblastoma with a murine IgG3 anti-GD2 antibody and GMC-SF (Cheung et al., 2006 J Clin Oncol 24(18):1-6). Murine IgG3 has higher affinity for the R131 isoform of human FcγRIIa than the H131 form, and patients homozygous for R131 showed better response than H/H homozygous patients. Notably, this is the first documentation of a clinical correlation between FcγR polymorphism and outcome in solid tumors, suggesting that the importance of FcγR-mediated effector functions is not limited to antibodies targeting hematological cancers.

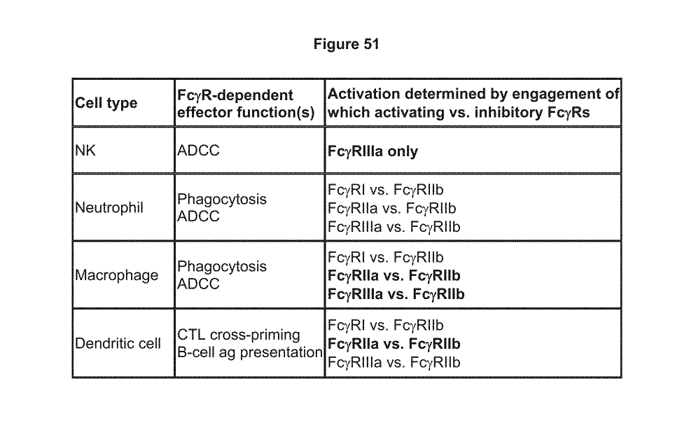

Together these data suggest that an antibody that is optimized for binding to certain FcγRs may better mediate effector functions and thereby destroy cancer cells more effectively in patients. Indeed progress has been made towards this goal, see for example U.S. Ser. No. 10/672,280, U.S. Ser. No. 10/822,231, U.S. Ser. No. 11/124,620, and U.S. Ser. No. 11/256,060. The majority of emphasis has thus far been directed at enhancing the affinity of antibodies for the activating receptor FcγRIIIa. However a major obstacle to improving antibody anti-tumor efficacy is engineering the proper balance between activating and inhibiting receptors. This is supported by the positive FcγRIIa polymorphism correlations with clinical outcome cited above because this receptor is virtually always expressed on immune cells along with the inhibitory receptor FcγRIIb. FIG. 51 shows the activating and inhibitory FcγRs that may be involved in regulating the activities of several immune cell types. Whereas NK cells only express the activating receptor FcγRIIIa, all of the other cell types, including neutrophils, macrophages, and dendritic cells, express the inhibitory receptor FcγRIIb, as well the other activating receptors FcγRI and FcγRIIa. For these cell types optimal effector function may result from an antibody that has increased affinity for activation receptors, for example FcγRI, FcγRIIa, and FcγRIIIa, yet reduced affinity for the inhibitory receptor FcγRIIb. Notably, these other cells types can utilitize FcγRs to mediate not only innate effector functions that directly lyse cells, for example ADCC, but can also phagocytose targeted cells and process antigen for presentation to other immune cells, events that can ultimately lead to the generation of adaptive immune response. For example, recent data suggest that the balance between FcγRIIa and FcγRIIb establishes a threshold of DC activation and enables immune complexes to mediate opposing effects on dendritic cell (DC) maturation and function (Boruchov et al., 2005, J Clin Invest., September 15, 1-10, entirely incorporated by reference). Thus, Fc variants that selectively ligate activating versus inhibitory receptors, for example FcγRIIa versus FcγRIIb, may affect DC processing, T cell priming and activation, antigen immunization, and/or efficacy against cancer (Dhodapkar & Dhodapkar, 2005, Proc Natl Acad Sci USA, 102, 6243-6244, entirely incorporated by reference). Such variants may be employed as novel strategies for targeting antigens to the activating or inhibitory FcγRs on human DCs, macrophages, or other antigen presenting cells to generate target-specific immunity.

In various aspects, the present application is directed to Fc variants having differential specificity for various receptors. For example, the change in affinity for one or more receptors can be increased relative to a second receptor or group of receptors.

In one aspect, the present invention is directed to an Fc variant of a parent Fc polypeptide comprising at least a first and a second substitution. The first and second substitutions are each at a position selected from group consisting of 234, 235, 236, 239, 267, 268, 293, 295, 324, 327, 328, 330, and 332 according to the EU index. The Fc variant exhibits an increase in affinity for one or more receptors selected from the group consisting of FcγRI, FcγRIIa, and FcγRIIIa as compared to the increase in a affinity of the Fc variant for the FcγRIIb receptor. The increases in affinities are relative to the parent polypeptide. In certain embodiments, the Fc variant has increased affinity for the activating receptor as compared to the parent Fc polypeptide but has reduced affinity (i.e., a negative increase in affinity) for FcγRIIb as compared to the parent Fc polypeptide. The increase in affinity is greater for an activating receptor than it is for FcγRIIb. Other activating receptors are also contemplated. In certain embodiments, the affinity for FcγRI, FcγRIIa, and FcγRIIIa receptors is increased.

Table A below illustrates several embodiments of human Fc receptor affinity profiles wherein the Fc variant provide selectively increased affinity for activating receptors relative to the inhibitory receptor FcγRIIb. One application of Fc variants with such Fc receptor affinity profiles is to impart antibodies, Fc fusions, or other Fc polypeptides with enhanced FcγR-mediated effector function and cellular activation, specifically for cells that express both activating and inhibitory receptors including but not limited to neutrophils, monocytes and macrophages, and dendritic cells.

TABLE A
Selectively increased affinity for activating receptors

| | FcγRI | FcγRIIa | FcγRIIb | FcγRIIIa |
|---|---|---|---|---|
| Embodiment 1 | + or WT | ++ | + | ++ |
| Embodiment 2 | + or WT | + | WT | + |
| Embodiment 3 | + or WT | + | − | + |

In another aspect, the Fc variant exhibits an increase in affinity of the Fc variant for the FcγRIIb receptor as compared to the increase in affinity for one or more activating receptors. Activating receptors include FcγRI, FcγRIIa, and FcγRIIIa. Increased affinities are relative to the parent polypeptide. The first and second substitutions each at a position selected from group consisting of 234, 235, 236, 239, 267, 268, 293, 295, 324, 327, 328, 330 and 332 according to the EU index. In other variations, the Fc variant has increased affinity for the activating receptor as compared to the parent Fc polypeptide but has reduced affinity (i.e., a negative increase in affinity) for FcγRIIb as compared to the parent Fc polypeptide. The increase in affinity is greater for FcγRIIb than it is for the one or more activating receptors. In further variations, the affinity for FcγRIIb is increased.

Table B below illustrates several embodiments of human Fc receptor affinity profiles wherein the Fc variant provide selectively increased affinity for the inhibitory receptor FcγRIIb relative to one or more activating receptors. One application of Fc variants with such Fc receptor affinity profiles is to impart antibodies, Fc fusions, or other Fc polypeptides with reduced FcγR-mediated effector function and to inhibit cellular activation, specifically for cells that express the inhibitory receptor FcγRIIb, including but not limited to neutrophils, monocytes and macrophages, dendritic cells, and B cells.

TABLE B
Selectively increased affinity for inhibitory receptor

| | FcγRI | FcγRIIa | FcγRIIb | FcγRIIIa |
|---|---|---|---|---|
| Embodiment 1 | + | + | ++ | + |
| Embodiment 2 | WT or − | WT or − | + | WT or − |
| Embodiment 3 | − | − | + | − |

In particular embodiments, the Fc variants that provide selectively increased affinity for activating receptors or inhibitory receptor are murine antibodies, and said selective enhancements are to murine Fc receptors. As described below in the examples, various embodiments provide for the generation of surrogate antibodies that are designed to be most compatible with mouse disease models, and may be informative for example in pre-clinical studies.

The presence of different polymorphic forms of FcγRs provides yet another parameter that impacts the therapeutic utility of the Fc variants of the present invention. Whereas the specificity and selectivity of a given Fc variant for the different classes of FcγRs significantly affects the capacity of an Fc variant to target a given antigen for treatment of a given disease, the specificity or selectivity of an Fc variant for different polymorphic forms of these receptors may in part determine which research or pre-clinical experiments may be appropriate for testing, and ultimately which patient populations may or may not respond to treatment. Thus, the specificity or selectivity of Fc variants of the present invention to Fc receptor polymorphisms, including but not limited to FcγRIIa, FcγRIIIa, and the like, may be used to guide the selection of valid research and pre-clinical experiments, clinical trial design, patient selection, dosing dependence, and/or other aspects concerning clinical trials.

Fc variants of the invention may comprise modifications that modulate interaction with Fc receptors other than FcγRs, including but not limited to complement proteins, FcRn, and Fc receptor homologs (FcRHs). FcRHs include but are not limited to FcRH1, FcRH2, FcRH3, FcRH4, FcRH5, and FcRH6 (Davis et al., 2002, Immunol. Reviews 190:123-136).

Modification may be made to improve the IgG stability, solubility, function, or clinical use. In a preferred embodiment, the IgG variants can include modifications to reduce immunogenicity in humans. In a most preferred embodiment, the immunogenicity of an IgG variant is reduced using a method described in U.S. Ser. No. 11/004,590, filed Dec. 3, 2004, entitled “Methods of Generating Variant Proteins with Increased Host String Content and Compositions Thereof”. In alternate embodiments, the IgG variants are humanized (Clark, 2000, Immunol Today 21:397-402).

Modifications to reduce immunogenicity can include modifications that reduce binding of processed peptides derived from the parent sequence to MHC proteins. For example, amino acid modifications would be engineered such that there are no or a minimal number of immune epitopes that are predicted to bind, with high affinity, to any prevalent MHC alleles. Several methods of identifying MHC-binding epitopes in protein sequences are known in the art and may be used to score epitopes in an IgG variant. See for example WO 98/52976; WO 02/079232; WO 00/3317; U.S. Ser. No. 09/903,378; U.S. Ser. No. 10/039,170; U.S. Ser. No. 60/222,697; U.S. Ser. No. 10/754,296; PCT WO 01/21823; and PCT WO 02/00165; Mallios, 1999, Bioinformatics 15: 432-439; Mallios, 2001, Bioinformatics 17: 942-948; Sturniolo et al., 1999, Nature Biotech. 17: 555-561; WO 98/59244; WO 02/069232; WO 02/77187; Marshall et al., 1995, J. Immunol. 154: 5927-5933; and Hammer et al., 1994, J. Exp. Med. 180: 2353-2358. Sequence-based information can be used to determine a binding score for a given peptide—MHC interaction (see for example Mallios, 1999, Bioinformatics 15: 432-439; Mallios, 2001, Bioinformatics 17: p 942-948; Sturniolo et. al., 1999, Nature Biotech. 17: 555-561).

Clearly an important parameter that determines the most beneficial selectivity of a given Fc variant to treat a given disease is the context of the Fc variant. Thus, the Fc receptor selectivity or specifity of a given Fc variant will provide different properties depending on whether it composes an antibody, Fc fusion, or Fc variants with a coupled fusion or conjugate partner. For example, toxin, radionucleotide, or other conjugates may be less toxic to normal cells if the IgG variant that comprises them has reduced or ablated binding to one or more Fc ligands. As another example, in order to inhibit inflammation or auto-immune disease, it may be preferable to utilize an IgG variant with enhanced affinity for activating FcγRs, such as to bind these FcγRs and prevent their activation. Conversely, an IgG variant that comprises two or more Fc regions with enhanced FcγRIIb affinity may co-engage this receptor on the surface of immune cells, thereby inhibiting proliferation of these cells. Whereas in some cases an IgG variants may engage its target antigen on one cell type yet engage FcγRs on separate cells from the target antigen, in other cases it may be advantageous to engage FcγRs on the surface of the same cells as the target antigen. For example, if an antibody targets an antigen on a cell that also expresses one or more FcγRs, it may be beneficial to utilize an IgG variant that enhances or reduces binding to the FcγRs on the surface of that cell. This may be the case, for example when the IgG variant is being used as an anti-cancer agent, and co-engagement of target antigen and FcγR on the surface of the same cell promote signaling events within the cell that result in growth inhibition, apoptosis, or other anti-proliferative effect. Alternatively, antigen and FcγR co-engagement on the same cell may be advantageous when the IgG variant is being used to modulate the immune system in some way, wherein co-engagement of target antigen and FcγR provides some proliferative or anti-proliferative effect. Likewise, IgG variants that comprise two or more Fc regions may benefit from IgG variants that modulate FcγR selectivity or specificity to co-engage FcγRs on the surface of the same cell.

Preferably, the Fc receptor specificity of the Fc variant of the present invention will determine its therapeutic utility. The utility of a given Fc variant for therapeutic purposes will depend on the epitope or form of the target antigen and the disease or indication being treated. For some targets and indications, enhanced FcγR-mediated effector functions may be preferable. This may be particularly favorable for anti-cancer Fc variants. Thus, Fc variants may be used that comprise Fc variants that provide enhanced affinity for activating FcγRs and/or reduced affinity for inhibitory FcγRs. For some targets and indications, it may be further beneficial to utilize Fc variants that provide differential selectivity for different activating FcγRs; for example, in some cases enhanced binding to FcγRIIa and FcγRIIIa may be desired, but not FcγR1, whereas in other cases, enhanced binding only to FcγRIIa may be preferred. For certain targets and indications, it may be preferable to utilize Fc variants that enhance both FcγR-mediated and complement-mediated effector functions, whereas for other cases it may be advantageous to utilize Fc variants that enhance either FcγR-mediated or complement-mediated effector functions. For some targets or cancer indications, it may be advantageous to reduce or ablate one or more effector functions, for example by knocking out binding to C1q, one or more FcγR selected from FcγR1, FcγRIIa, FcγRIIb, FcγRIIc, FcγRIIIa, and FcγRIIIb, FcRn, or one or more other Fc ligands. For other targets and indications, it may be preferable to utilize Fc variants that provide enhanced binding to the inhibitory FcγRIIb, yet WT level, reduced, or ablated binding to activating FcγRs. This may be particularly useful, for example, when the goal of an Fc variant is to inhibit inflammation or auto-immune disease, or modulate the immune system in some way.

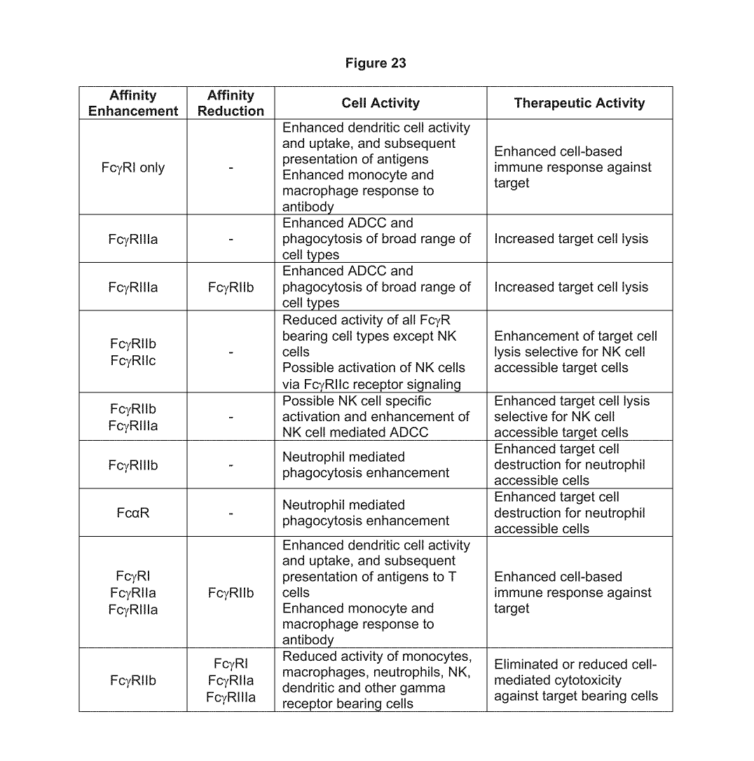

The Fc ligand specificity of the IgG variants can be modulated to create different effector function profiles that may be suited for particular target antigens, indications, or patient populations. FIG. 23 describes several preferred embodiments of receptor binding profiles that include improvements to, reductions to or no effect to the binding to various receptors, where such changes may be beneficial in certain contexts. The receptor binding profiles in the table could be varied by degree of increase or decrease to the specified receptors. Additionally, the binding changes specified could be in the context of additional binding changes to other receptors such as C1q or FcRn, for example by combining with ablation of binding to C1q to shut off complement activation, or by combining with enhanced binding to C1q to increase complement activation. Other embodiments with other receptor binding profiles are possible, the listed receptor binding profiles are exemplary.

In a preferred embodiment, the target of the Fc variants of the present invention is itself one or more Fc ligands. Fc polypeptides of the invention can be utilized to modulate the activity of the immune system, and in some cases to mimic the effects of IVIg therapy in a more controlled, specific, and efficient manner. IVIg is effectively a high dose of immunoglobulins delivered intravenously. In general, IVIg has been used to downregulate autoimmune conditions. It has been hypothesized that the therapeutic mechanism of action of IVIg involves ligation of Fc receptors at high frequency (J. Bayry et al., 2003, Transfusion Clinique et Biologique 10: 165-169; Binstadt et al., 2003, J Allergy Clin. Immunol, 697-704). Indeed animal models of Ithrombocytopenia purpura (ITP) show that the isolated Fc are the active portion of IVIg (Samuelsson et al, 200I, Pediatric Research 50(5), 551). For use in therapy, iimmunoglobulins are harvested from thousands of donors, with all of the concomitant problems associated with non-recombinant biotherapeutics collected from humans. An Fc variant of the present invention should serve all of the roles of IVIg while being manufactured as a recombinant protein rather than harvested from donors.

The immunomodulatory effects of IVIg may be dependent on productive interaction with one or more Fc ligands, including but not limited to FcγRs, complement proteins, and FcRn. In some embodiments, Fc variants of the invention with enhanced affinity for FcγRIIb can be used to promote anti-inflammatory activity (Samuelsson et al., 2001, Science 291: 484-486) and or to reduce autoimmunity (Hogarth, 2002, Current Opinion in Immunology, 14:798-802). In other embodiments, Fc polypeptides of the invention with enhanced affinity for one or more FcγRs can be utilized by themselves or in combination with additional modifications to reduce autoimmunity (Hogarth, 2002, Current Opinion in Immunology, 14:798-802). In alternative embodiments, Fc variants of the invention with enhanced affinity for FcγRIIIa but reduced capacity for intracellular signaling can be used to reduce immune system activation by competitively interfering with FcγRIIIa binding. The context of the Fc variant drammatically impacts the desired specificity. For example, Fc variants that provide enhanced binding to one or more activating FcγRs may provide optimal immunomodulatory effects in the context of an antibody, Fc fusion, isolated Fc, or Fc fragment by acting as an FcγR antagonist (van Mirre et al., 2004, J. Immunol. 173:332-339). However, fusion or conjugation of two or more Fc variants may provide different effects, and for such an Fc polypeptide it may be optimal to utilize Fc variants that provide enhanced affinity for an inhibitory receptor.

The Fc variants of the present invention may be used as immunomodulatory therapeutics. Binding to or blocking Fc receptors on immune system cells may be used to influence immune response in immunological conditions including but not limited to idiopathic thrombocytopenia purpura (ITP) and rheumatoid arthritis (RA) among others. By use of the affinity enhanced Fc variants of the present invention, the dosages required in typical IVIg applications may be reduced while obtaining a substantially similar therapeutic effect. The Fc variants may provide enhanced binding to an FcγR, including but not limited to FcγRIIa, FcγRIIb, FcγRIIIa, FcγRIIIb, and/or FcγRI. In particular, binding enhancements to FcγRIIb would increase expression or inhibitory activity, as needed, of that receptor and improve efficacy. Alternatively, blocking binding to activation receptors such as FcγRIIIb or FcγRI may improve efficacy. In addition, modulated affinity of the Fc variants for FcRn and/or also complement may also provide benefits.

In one embodiment, Fc variants that provide enhanced binding to the inhibitory receptor FcγRIIb provide an enhancement to the IVIg therapeutic approach. In particular, the Fc variants of the present invention that bind with greater affinity to the FcγRIIb receptor than parent Fc polypeptide may be used. Such Fc variants would thus function as FcγRIIb agonists, and would be expected to enhance the beneficial effects of IVIg as an autoimmune disease therapeutic and also as a modulator of B-cell proliferation. In addition, such FcγRIIb-enhanced Fc variants may also be further modified to have the same or limited binding to other receptors. In additional embodiments, the Fc variants with enhanced FcγRIIb affinity may be combined with mutations that reduce or ablate to other receptors, thereby potentially further minimizing side effects during therapeutic use.

Such immunomodulatory applications of the Fc variants of the present invention may also be utilized in the treatment of oncological indications, especially those for which antibody therapy involves antibody-dependant cytotoxic mechanisms. For example, an Fc variant that enhances affinity to FcγRIIb may be used to antagonize this inhibitory receptor, for example by binding to the Fc/FcγRIIb binding site but failing to trigger, or reducing cell signaling, potentially enhancing the effect of antibody-based anti-cancer therapy. Such Fc variants, functioning as FcγRIIb antagonists, may either block the inhibitory properties of FcγRIIb, or induce its inhibitory function as in the case of IVIg. An FcγRIIb antagonist may be used as co-therapy in combination with any other therapeutic, including but not limited to antibodies, acting on the basis of ADCC related cytotoxicity. FcγRIIb antagonistic Fc variants of this type are preferably isolated Fc or Fc fragments, although in alternate embodiments antibodies and Fc fusions may be used.

### Additional Modifications

In addition to the modifications outlined above, other modifications can be made. For example, the molecules may be stabilized by the incorporation of disulphide bridges linking the VH and VL domains (Reiter et al., 1996, Nature Biotech. 14:1239-1245, entirely incorporated by reference). In addition, there are a variety of covalent modifications of antibodies that can be made as outlined below.

Covalent modifications of antibodies are included within the scope of this invention, and are generally, but not always, done post-translationally. For example, several types of covalent modifications of the antibody are introduced into the molecule by reacting specific amino acid residues of the antibody with an organic derivatizing agent that is capable of reacting with selected side chains or the N- or C-terminal residues.

Cysteinyl residues most commonly are reacted with α-haloacetates (and corresponding amines), such as chloroacetic acid or chloroacetamide, to give carboxymethyl or carboxyamidomethyl derivatives. Cysteinyl residues may also be derivatized by reaction with bromotrifluoroacetone, α-bromo-β-(5-imidozoyl)propionic acid, chloroacetyl phosphate, N-alkylmaleimides, 3-nitro-2-pyridyl disulfide, methyl 2-pyridyl disulfide, p-chloromercuribenzoate, 2-chloromercuri-4-nitrophenol, or chloro-7-nitrobenzo-2-oxa-1,3-diazole and the like.

In addition, modifications at cysteines are particularly useful in antibody-drug conjugate (ADC) applications, further described below. In some embodiments, the constant region of the antibodies can be engineered to contain one or more cysteines that are particularly “thiol reactive”, so as to allow more specific and controlled placement of the drug moiety. See for example U.S. Pat. No. 7,521,541, incorporated by reference in its entirety herein.

Histidyl residues are derivatized by reaction with diethylpyrocarbonate at pH 5.5-7.0 because this agent is relatively specific for the histidyl side chain. Para-bromophenacyl bromide also is useful; the reaction is preferably performed in 0.1M sodium cacodylate at pH 6.0.

Lysinyl and amino terminal residues are reacted with succinic or other carboxylic acid anhydrides. Derivatization with these agents has the effect of reversing the charge of the lysinyl residues. Other suitable reagents for derivatizing alpha-amino-containing residues include imidoesters such as methyl picolinimidate; pyridoxal phosphate; pyridoxal; chloroborohydride; trinitrobenzenesulfonic acid; O-methylisourea; 2,4-pentanedione; and transaminase-catalyzed reaction with glyoxylate.

Arginyl residues are modified by reaction with one or several conventional reagents, among them phenylglyoxal, 2,3-butanedione, 1,2-cyclohexanedione, and ninhydrin. Derivatization of arginine residues requires that the reaction be performed in alkaline conditions because of the high pKa of the guanidine functional group. Furthermore, these reagents may react with the groups of lysine as well as the arginine epsilon-amino group.

The specific modification of tyrosyl residues may be made, with particular interest in introducing spectral labels into tyrosyl residues by reaction with aromatic diazonium compounds or tetranitromethane. Most commonly, N-acetylimidizole and tetranitromethane are used to form O-acetyl tyrosyl species and 3-nitro derivatives, respectively. Tyrosyl residues are iodinated using 125I or 131I to prepare labeled proteins for use in radioimmunoassay, the chloramine T method described above being suitable.

Carboxyl side groups (aspartyl or glutamyl) are selectively modified by reaction with carbodiimides (R′—N═C═N—R′), where R and R′ are optionally different alkyl groups, such as 1-cyclohexyl-3-(2-morpholinyl-4-ethyl) carbodiimide or 1-ethyl-3-(4-azonia-4,4-dimethylpentyl) carbodiimide. Furthermore, aspartyl and glutamyl residues are converted to asparaginyl and glutaminyl residues by reaction with ammonium ions.

Derivatization with bifunctional agents is useful for crosslinking antibodies to a water-insoluble support matrix or surface for use in a variety of methods, in addition to methods described below. Commonly used crosslinking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis (succinimidylpropionate), and bifunctional maleimides such as bis-N-maleimido-1,8-octane. Derivatizing agents such as methyl-3-[(p-azidophenyl)dithio]propioimidate yield photoactivatable intermediates that are capable of forming crosslinks in the presence of light. Alternatively, reactive water-insoluble matrices such as cynomolgusogen bromide-activated carbohydrates and the reactive substrates described in U.S. Pat. Nos. 3,969,287; 3,691,016; 4,195,128; 4,247,642; 4,229,537; and 4,330,440, all entirely incorporated by reference, are employed for protein immobilization.

Glutaminyl and asparaginyl residues are frequently deamidated to the corresponding glutamyl and aspartyl residues, respectively. Alternatively, these residues are deamidated under mildly acidic conditions. Either form of these residues falls within the scope of this invention.

Other modifications include hydroxylation of proline and lysine, phosphorylation of hydroxyl groups of seryl or threonyl residues, methylation of the α-amino groups of lysine, arginine, and histidine side chains (T. E. Creighton, Proteins: Structure and Molecular Properties, W. H. Freeman & Co., San Francisco, pp. 79-86 [1983], entirely incorporated by reference), acetylation of the N-terminal amine, and amidation of any C-terminal carboxyl group.

In addition, as will be appreciated by those in the art, labels (including fluorescent, enzymatic, magnetic, radioactive, etc. can all be added to the antibodies (as well as the other compositions of the invention).

### Glycosylation

Another type of covalent modification is alterations in glycosylation. In another embodiment, the antibodies disclosed herein can be modified to include one or more engineered glycoforms. By “engineered glycoform” as used herein is meant a carbohydrate composition that is covalently attached to the antibody, wherein said carbohydrate composition differs chemically from that of a parent antibody. Engineered glycoforms may be useful for a variety of purposes, including but not limited to enhancing or reducing effector function. A preferred form of engineered glycoform is afucosylation, which has been shown to be correlated to an increase in ADCC function, presumably through tighter binding to the FcγRIIIa receptor. In this context, “afucosylation” means that the majority of the antibody produced in the host cells is substantially devoid of fucose, e.g., 90-95-98% of the generated antibodies do not have appreciable fucose as a component of the carbohydrate moiety of the antibody (generally attached at N297 in the Fc region). Defined functionally, afucosylated antibodies generally exhibit at least a 50% or higher affinity to the FcγRIIIa receptor.

Engineered glycoforms may be generated by a variety of methods known in the art (Umaña et al., 1999, Nat Biotechnol 17:176-180; Davies et al., 2001, Biotechnol Bioeng 74:288-294; Shields et al., 2002, J Biol Chem 277:26733-26740; Shinkawa et al., 2003, J Biol Chem 278:3466-3473; U.S. Pat. No. 6,602,684; U.S. Ser. No. 10/277,370; U.S. Ser. No. 10/113,929; PCT WO 00/61739A1; PCT WO 01/29246A1; PCT WO 02/31140A1; PCT WO 02/30954A1, all entirely incorporated by reference; (Potelligent® technology [Biowa, Inc., Princeton, N.J.]; GlycoMAb® glycosylation engineering technology [Glycart Biotechnology AG, Zurich, Switzerland]). Many of these techniques are based on controlling the level of fucosylated and/or bisecting oligosaccharides that are covalently attached to the Fc region, for example by expressing an IgG in various organisms or cell lines, engineered or otherwise (for example Lec-13 CHO cells or rat hybridoma YB2/0 cells, by regulating enzymes involved in the glycosylation pathway (for example FUT8 [α1,6-fucosyltranserase] and/or β1-4-

N-acetylglucosaminyltransferase III [GnTIII]), or by modifying carbohydrate(s) after the IgG has been expressed. For example, the “sugar engineered antibody” or “SEA technology” of Seattle Genetics functions by adding modified saccharides that inhibit fucosylation during production; see for example 20090317869, hereby incorporated by reference in its entirety. Engineered glycoform typically refers to the different carbohydrate or oligosaccharide; thus, an antibody can include an engineered glycoform.

Alternatively, engineered glycoform may refer to the IgG variant that comprises the different carbohydrate or oligosaccharide. As is known in the art, glycosylation patterns can depend on both the sequence of the protein (e.g., the presence or absence of particular glycosylation amino acid residues, discussed below), or the host cell or organism in which the protein is produced. Particular expression systems are discussed below.

Glycosylation of polypeptides is typically either N-linked or O-linked. N-linked refers to the attachment of the carbohydrate moiety to the side chain of an asparagine residue. The tri-peptide sequences asparagine-X-serine and asparagine-X-threonine, where X is any amino acid except proline, are the recognition sequences for enzymatic attachment of the carbohydrate moiety to the asparagine side chain. Thus, the presence of either of these tri-peptide sequences in a polypeptide creates a potential glycosylation site. O-linked glycosylation refers to the attachment of one of the sugars N-acetylgalactosamine, galactose, or xylose, to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tri-peptide sequences (for N-linked glycosylation sites). The alteration may also be made by the addition of, or substitution by, one or more serine or threonine residues to the starting sequence (for O-linked glycosylation sites). For ease, the antibody amino acid sequence is preferably altered through changes at the DNA level, particularly by mutating the DNA encoding the target polypeptide at preselected bases such that codons are generated that will translate into the desired amino acids.

Another means of increasing the number of carbohydrate moieties on the antibody is by chemical or enzymatic coupling of glycosides to the protein. These procedures are advantageous in that they do not require production of the protein in a host cell that has glycosylation capabilities for N- and O-linked glycosylation. Depending on the coupling mode used, the sugar(s) may be attached to (a) arginine and histidine, (b) free carboxyl groups, (c) free sulfhydryl groups such as those of cysteine, (d) free hydroxyl groups such as those of serine, threonine, or hydroxyproline, (e) aromatic residues such as those of phenylalanine, tyrosine, or tryptophan, or (f) the amide group of glutamine. These methods are described in WO 87/05330 and in Aplin and Wriston, 1981, CRC Crit. Rev. Biochem., pp. 259-306, both entirely incorporated by reference.

Removal of carbohydrate moieties present on the starting antibody (e.g., post-translationally) may be accomplished chemically or enzymatically. Chemical deglycosylation requires exposure of the protein to the compound trifluoromethanesulfonic acid, or an equivalent compound. This treatment results in the cleavage of most or all sugars except the linking sugar (N-acetylglucosamine or N-acetylgalactosamine), while leaving the polypeptide intact. Chemical deglycosylation is described by Hakimuddin et al., 1987, Arch. Biochem. Biophys. 259:52 and by Edge et al., 1981, Anal. Biochem. 118:131, both entirely incorporated by reference. Enzymatic cleavage of carbohydrate moieties on polypeptides can be achieved by the use of a variety of endo- and exo-glycosidases as described by Thotakura et al., 1987, Meth. Enzymol. 138:350, entirely incorporated by reference. Glycosylation at potential glycosylation sites may be prevented by the use of the compound tunicamycin as described by Duskin et al., 1982, J. Biol. Chem. 257:3105, entirely incorporated by reference. Tunicamycin blocks the formation of protein-N-glycoside linkages.

Another type of covalent modification of the antibody comprises linking the antibody to various nonproteinaceous polymers, including, but not limited to, various polyols such as polyethylene glycol, polypropylene glycol or polyoxyalkylenes, in the manner set forth in, for example, 2005-2006 PEG Catalog from Nektar Therapeutics (available at the Nektar website) U.S. Pat. Nos. 4,640,835; 4,496,689; 4,301,144; 4,670,417; 4,791,192 or 4,179,337, all entirely incorporated by reference. In addition, as is known in the art, amino acid substitutions may be made in various positions within the antibody to facilitate the addition of polymers such as PEG. See for example, U.S. Publication No. 2005/0114037A1, entirely incorporated by reference.

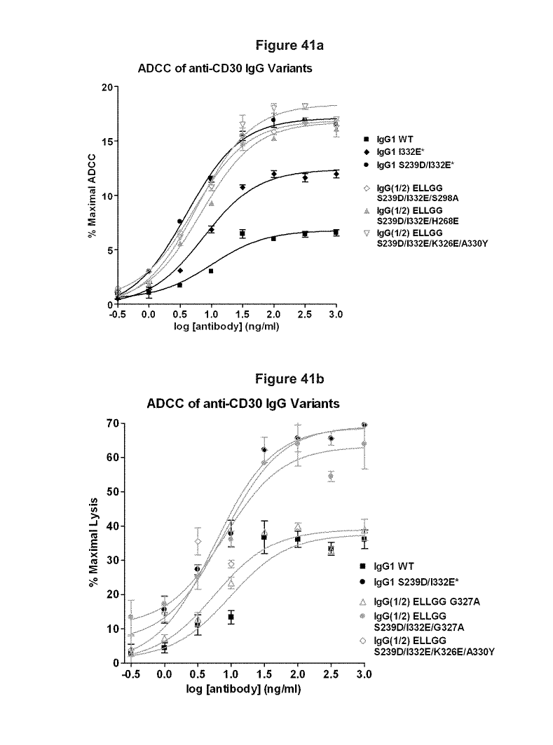

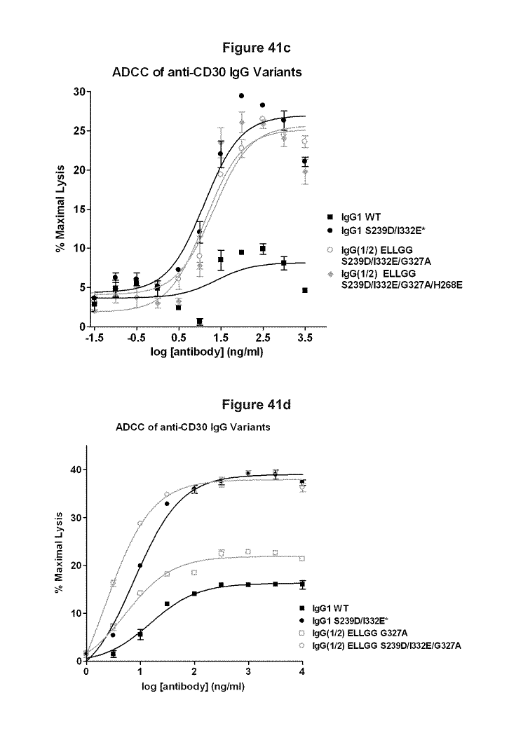

In one embodiment, the Fc polypeptides of the invention can include amino acid modifications to alter binding to one or more of the FcγR receptors. Substitutions that result in increased binding as well as decreased binding can be useful. For example, it is known that increased binding to FcγRIIIa generally results in increased ADCC (antibody dependent cell-mediated cytotoxicity; the cell-mediated reaction wherein nonspecific cytotoxic cells that express FcγRs recognize bound antibody on a target cell and subsequently cause lysis of the target cell). Similarly, decreased binding to FcγRIIb (an inhibitory receptor) can be beneficial as well in some circumstances. Amino acid substitutions that find use in the present invention include those listed in U.S. Ser. No. 11/124,620 (particularly FIG. 41, specifically incorporated herein), Ser. Nos. 11/174,287, 11/396,495, 11/538,406, all of which are expressly incorporated herein by reference in their entirety and specifically for the variants disclosed therein.

Particular variants that find use include, but are not limited to, 236A, 239D, 239E, 332E, 332D, 239D/332E, 267D, 267E, 328F, 267E/328F, 236A/332E, 239D/332E/330Y, 239D, 332E/330L, 243L, 236R, 328R, 236R/328R and 299T. Additional suitable Fc variants are found in FIG. 41 of US 2006/0024298, the figure and legend of which are hereby incorporated by reference in their entirety.

### Binding Moieties/Targets

The proteins (for example the immunoglobulins) of the invention may target virtually any antigens. As noted above, there are a wide variety of suitable antibody formats.

Particular suitable applications of the immunoglobulins herein are co-target pairs for which it is beneficial or critical to engage a target antigen monovalently. Such antigens may be, for example, immune receptors that are activated upon immune complexation. Cellular activation of many immune receptors occurs only by cross-linking, achieved typically by antibody/antigen immune complexes, or via effector cell to target cell engagement. For some immune receptors, for example the CD3 signaling receptor on T cells, activation only upon engagement with co-engaged target is critical, as nonspecific cross-linking in a clinical setting can elicit a cytokine storm and toxicity. Therapeutically, by engaging such antigens monovalently rather than multivalently, using the immunoglobulins herein, such activation occurs only in response to cross-linking only in the microenvironment of the primary target antigen. The ability to target two different antigens with different valencies is a novel and useful aspect of the present invention. Examples of target antigens for which it may be therapeutically beneficial or necessary to co-engage monovalently include but are not limited to immune activating receptors such as CD3, FcγRs, toll-like receptors (TLRs) such as TLR4 and TLR9, cytokine, chemokine, cytokine receptors, and chemokine receptors.

Virtually any antigen may be targeted by the immunoglobulins herein, including but not limited to proteins, subunits, domains, motifs, and/or epitopes belonging to the following list of target antigens, which includes both soluble factors such as cytokines and membrane-bound factors, including transmembrane receptors: 17-IA, 4-1BB, 4Dc, 6-keto-PGF1a, 8-iso-PGF2a, 8-oxo-dG, α4-integrin, A1 Adenosine Receptor, A33, ACE, ACE-2, Activin, Activin A, Activin AB, Activin B, Activin C, Activin RIA, Activin RIA ALK-2, Activin RIB ALK-4, Activin RIIA, Activin RIIB, ADAM, ADAM10, ADAM12, ADAM15, ADAM17/TACE, ADAM8, ADAM9, ADAMTS, ADAMTS4, ADAMTS5, Addressins, aFGF, ALCAM, ALK, ALK-1, ALK-7, alpha-1-antitrypsin, alpha-V/beta-1 antagonist, ANG, Ang, APAF-1, APE, APJ, APP, APRIL, AR, ARC, ART, Artemin, anti-Id, ASPARTIC, Atrial natriuretic factor, av/b3 integrin, Ax1, b2M, B7-1, B7-2, B7-H, B. anthrasis PA, B-lymphocyte Stimulator (BlyS), BACE, BACE-1, Bad, BAFF, BAFF-R, Bag-1, BAK, Bax, BCA-1, BCAM, Bcl, BCMA, BDNF, b-ECGF, bFGF, BID, Bik, BIM, BLC, BL-CAM, BLK, BLyS, BMP, BMP-2 BMP-2a, BMP-3 Osteogenin, BMP-4 BMP-2b, BMP-5, BMP-6 Vgr-1, BMP-7 (OP-1), BMP-8 (BMP-8a, OP-2), BMPR, BMPR-IA (ALK-3), BMPR-IB (ALK-6), BRK-2, RPK-1, BMPR-II (BRK-3), BMPs, b-NGF, BOK, Bombesin, Bone-derived neurotrophic factor, BPDE, BPDE-DNA, BTC, complement factor 3 (C3), C3a, C4, C5, C5a, C10, CA125, CAD-8, Calcitonin, cAMP, carcinoembryonic antigen (CEA), carcinoma-associated antigen, Cathepsin A, Cathepsin B, Cathepsin C/DPPI, Cathepsin D, Cathepsin E, Cathepsin H, Cathepsin L, Cathepsin O, Cathepsin S, Cathepsin V, Cathepsin X/Z/P, C5, CBL, CCI, CCK2, CCL, CCL1, CCL11, CCL12, CCL13, CCL14, CCL15, CCL16, CCL17, CCL18, CCL19, CCL2, CCL20, CCL21, CCL22, CCL23, CCL24, CCL25, CCL26, CCL27, CCL28, CCL3, CCL4, CCL5, CCL6, CCL7, CCL8, CCL9/10, CCR, CCR1, CCR4, CCR10, CCR10, CCR2, CCR3, CCR4, CCR5, CCR6, CCR7, CCR8, CCR9, CD1, CD2, CD3, CD3E, CD4, CD5, CD6, CD7, CD8, CD10, CD11a, CD11b, CD11c, CD13, CD14, CD15, CD16, CD18, CD19, CD20, CD21, CD22, CD23, CD25, CD27L, CD28, CD29, CD30, CD30L, CD32, CD33 (p67 proteins), CD34, CD38, CD40, CD40L, CD44, CD45, CD46, CD49a, CD52, CD54, CD55, CD56, CD61, CD64, CD66e, CD74, CD80 (B7-1), CD89, CD95, CD123, CD137, CD138, CD140a, CD146, CD147, CD148, CD152, CD164, CEACAM5, CFTR, cGMP, CINC, Clostridium botulinum toxin, Clostridium perfringens toxin, CKb8-1, CLC, CMV, CMV UL, CNTF, CNTN-1, COX, C-Ret, CRG-2, CT-1, CTACK, CTGF, CTLA-4, CX3CL1, CX3CR1, CXCL, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCR, CXCR1, CXCR2, CXCR3, CXCR4, CXCR5, CXCR6, cytokeratin tumor-associated antigen, DAN, DCC, DcR3, DC-SIGN, Decay accelerating factor, des(1-3)-IGF-I (brain IGF-1), Dhh, digoxin, DNAM-1, Dnase, Dpp, DPPIV/CD26, Dtk, ECAD, EDA, EDA-A1, EDA-A2, EDAR, EGF, EGFR (ErbB-1), EMA, EMMPRIN, ENA, endothelin receptor, endotoxin, Enkephalinase, eNOS, Eot, eotaxin1, EpCAM, Ephrin B2/EphB4, EPO, ERCC, E-selectin, ET-1, Factor IIa, Factor VII, Factor VIIIc, Factor IX, fibroblast activation protein (FAP), Fas, FcR1, FEN-1, Ferritin, FGF, FGF-19, FGF-2, FGF3, FGF-8, FGFR, FGFR-3, Fibrin, FL, FLIP, Flt-3, Flt-4, Follicle stimulating hormone, Fractalkine, FZD1, FZD2, FZD3, FZD4, FZD5, FZD6, FZD7, FZD8, FZD9, FZD10, G250, Gas 6, GCP-2, GCSF, GD2, GD3, GDF, GDF-1, GDF-3 (Vgr-2), GDF-5 (BMP-14, CDMP-1), GDF-6 (BMP-13, CDMP-2), GDF-7 (BMP-12, CDMP-3), GDF-8 (Myostatin), GDF-9, GDF-15 (MIC-1), GDNF, GDNF, GFAP, GFRa-1, GFR-alpha1, GFR-alpha2, GFR-alpha3, GITR, Glucagon, Glut 4, glycoprotein IIb/IIIa (GP IIb/IIIa), GM-CSF, GPIIb/IIIa, gp130, gp72, GRO, Growth hormone releasing factor, Hapten (NP-cap or NIP-cap), HB-EGF, HCC, HCMV gB envelope glycoprotein, HCMV) gH envelope glycoprotein, HCMV UL, Hemopoietic growth factor (HGF), Hep B gp120, heparanase, Her2, Her2/neu (ErbB-2), Her3 (ErbB-3), Her4 (ErbB-4), herpes simplex virus (HSV) gB glycoprotein, HSV gD glycoprotein, HGFA, High molecular weight melanoma-associated antigen (HMW-MAA), HIV gp120, HIV IIIB gp 120 V3 loop, HLA, HLA-DR, HM1.24, HMFG PEM, HRG, Hrk, human cardiac myosin, human cytomegalovirus (HCMV), human growth hormone (HGH), HVEM, 1-309, IAP, ICAM, ICAM-1, ICAM-3, ICE, ICOS, IFNg, Ig, IgA receptor, IgE, IGF, IGF binding proteins, IGF-1R, IGFBP, IGF-I, IGF-II, IL, IL-1, IL-1b, IL-1R, IL-2, IL-2R, IL-4, IL-4R, IL-5, IL-5R, IL-6, IL-6R, IL-8, IL-9, IL-10, IL-12, IL-13, IL-15, IL-18, IL-18R, IL-23, interferon (INF)-alpha, INF-beta, INF-gamma, Inhibin, iNOS, Insulin A-chain, Insulin B-chain, Insulin-like growth factor 1, integrin alpha2, integrin alpha3, integrin alpha4, integrin alpha4/beta1, integrin alpha4/beta7, integrin alpha5 (alphaV), integrin alpha5/beta1, integrin alpha5/beta3, integrin alpha6, integrin beta1, integrin beta2, interferon gamma, IP-10, I-TAC, JE, Kallikrein 2, Kallikrein 5, Kallikrein 6, Kallikrein 1I, Kallikrein 12, Kallikrein 14, Kallikrein 15, Kallikrein L1, Kallikrein L2, Kallikrein L3, Kallikrein L4, KC, KDR, Keratinocyte Growth Factor (KGF), laminin 5, LAMP, LAP, LAP (TGF- 1), Latent TGF-1, Latent TGF-1 bp1, LBP, LDGF, LECT2, Lefty, Lewis-Y antigen, Lewis-Y related antigen, LFA-1, LFA-3, Lfo, LIF, LIGHT, lipoproteins, LIX, LKN, Lptn, L-Selectin, LT-a, LT-b, LTB4, LTBP-1, Lung surfactant, Luteinizing hormone, Lymphotoxin Beta Receptor, Mac-1, MAdCAM, MAG, MAP2, MARC, MCAM, MCAM, MCK-2, MCP, M-CSF, MDC, Mer, METALLOPROTEASES, MGDF receptor, MGMT, MHC (HLA-DR), MIF, MIG, MIP, MIP-1-alpha, MK, MMAC1, MMP, MMP-1, MMP-10, MMP-11, MMP-12, MMP-13, MMP-14, MMP-15, MMP-2, MMP-24, MMP-3, MMP-7, MMP-8, MMP-9, MPIF, Mpo, MSK, MSP, mucin (Muc1), MUC18, Muellerian-inhibitin substance, Mug, MuSK, NAIP, NAP, NCAD, N-Cadherin, NCA 90, NCAM, NCAM, Neprilysin, Neurotrophin-3, -4, or -6, Neurturin, Neuronal growth factor (NGF), NGFR, NGF-beta, nNOS, NO, NOS, Npn, NRG-3, NT, NTN, OB, OGG1, OPG, OPN, OSM, OX40L, OX40R, p150, p95, PADPr, Parathyroid hormone, PARC, PARP, PBR, PBSF, PCAD, P-Cadherin, PCNA, PDGF, PDGF, PDK-1, PECAM, PEM, PF4, PGE, PGF, PGI2, PGJ2, PIN, PLA2, placental alkaline phosphatase (PLAP), P1GF, PLP, PP14, Proinsulin, Prorelaxin, Protein C, PS, PSA, PSCA, prostate specific membrane antigen (PSMA), PTEN, PTHrp, Ptk, PTN, R51, RANK, RANKL, RANTES, RANTES, Relaxin A-chain, Relaxin B-chain, renin, respiratory syncytial virus (RSV) F, RSV Fgp, Ret, Rheumatoid factors, RLIP76, RPA2, RSK, RSV, S100, SCF/KL, SDF-1, SERINE, Serum albumin, sFRP-3, Shh, SIGIRR, SK-1, SLAM, SLPI, SMAC, SMDF, SMOH, SOD, SPARC, Stat, STEAP, STEAP-II, TACE, TACI, TAG-72 (tumor-associated glycoprotein-72), TARC, TCA-3, T-cell receptors (e.g., T-cell receptor alpha/beta), TdT, TECK, TEM1, TEM5, TEM7, TEM8, TERT, testicular PLAP-like alkaline phosphatase, TfR, TGF, TGF-alpha, TGF-beta, TGF-beta Pan Specific, TGF-beta RI (ALK-5), TGF-beta RII, TGF-beta RIIb, TGF-beta RIII, TGF-beta1, TGF-beta2, TGF-beta3, TGF-beta4, TGF-beta5, Thrombin, Thymus Ck-1, Thyroid stimulating hormone, Tie, TIMP, TIQ, Tissue Factor, TMEFF2, Tmpo, TMPRSS2, TNF, TNF-alpha, TNF-alpha beta, TNF-beta2, TNFc, TNF-RI, TNF-RII, TNFRSF10A (TRAIL R1 Apo-2, DR4), TNFRSF10B (TRAIL R2 DR5, KILLER, TRICK-2A, TRICK-B), TNFRSF10C (TRAIL R3 DcR1, LIT, TRID), TNFRSF10D (TRAIL R4 DcR2, TRUNDD), TNFRSF11A (RANK ODF R, TRANCE R), TNFRSF11B (OPG OCIF, TR1), TNFRSF12 (TWEAK R FN14), TNFRSF13B (TACI), TNFRSF13C (BAFF R), TNFRSF14 (HVEM ATAR, HveA, LIGHT R, TR2), TNFRSF16 (NGFR p75NTR), TNFRSF17 (BCMA), TNFRSF18 (GITR AITR), TNFRSF19 (TROY TAJ, TRADE), TNFRSF19L (RELT), TNFRSF1A (TNF RI CD120a, p55-60), TNFRSF1B (TNF RII CD120b, p75-80), TNFRSF26 (TNFRH3), TNFRSF3 (LTbR TNF RIII, TNFC R), TNFRSF4 (OX40 ACT35, TXGP1 R), TNFRSF5 (CD40 p50), TNFRSF6 (Fas Apo-1, APT1, CD95), TNFRSF6B (DcR3 M68, TR6), TNFRSF7 (CD27), TNFRSF8 (CD30), TNFRSF9 (4-1BB CD137, ILA), TNFRSF21 (DR6), TNFRSF22 (DcTRAIL R2 TNFRH2), TNFRST23 (DcTRAIL R1 TNFRH1), TNFRSF25 (DR3 Apo-3, LARD, TR-3, TRAMP, WSL-1), TNFSF10 (TRAIL Apo-2 Ligand, TL2), TNFSF11 (TRANCE/RANK Ligand ODF, OPG Ligand), TNFSF12 (TWEAK Apo-3 Ligand, DR3 Ligand), TNFSF13 (APRIL TALL2), TNFSF13B (BAFF BLYS, TALL1, THANK, TNFSF20), TNFSF14 (LIGHT HVEM Ligand, LTg), TNFSF15 (TL1A/VEGI), TNFSF18 (GITR Ligand AITR Ligand, TL6), TNFSF1A (TNF-a Conectin, DIF, TNFSF2), TNFSF1B (TNF-b LTa, TNFSF1), TNFSF3 (LTb TNFC, p33), TNFSF4 (OX40 Ligand gp34, TXGP1), TNFSF5 (CD40 Ligand CD154, gp39, HIGM1, IMD3, TRAP), TNFSF6 (Fas Ligand Apo-1 Ligand, APT1 Ligand), TNFSF7 (CD27 Ligand CD70), TNFSF8 (CD30 Ligand CD153), TNFSF9 (4-1BB Ligand CD137 Ligand), TP-1, t-PA, Tpo, TRAIL, TRAIL R, TRAIL-R1, TRAIL-R2, TRANCE, transferring receptor, TRF, Trk, TROP-2, TSG, TSLP, tumor-associated antigen CA 125, tumor-associated antigen expressing Lewis Y related carbohydrate, TWEAK, TXB2, Ung, uPAR, uPAR-1, Urokinase, VCAM, VCAM-1, VECAD, VE-Cadherin, VE-cadherin-2, VEFGR-1 (flt-1), VEGF, VEGFR, VEGFR-3 (flt-4), VEGI, VIM, Viral antigens, VLA, VLA-1, VLA-4, VNR integrin, von Willebrands factor, WIF-1, WNT1, WNT2, WNT2B/13, WNT3, WNT3A, WNT4, WNT5A, WNT5B, WNT6, WNT7A, WNT7B, WNT8A, WNT8B, WNT9A, WNT9A, WNT9B, WNT10A, WNT10B, WNT11, WNT16, XCL1, XCL2, XCR1, XCR1, XEDAR, XIAP, XPD, and receptors for hormones and growth factors.

Exemplary antigens that may be targeted specifically by the immunoglobulins of the invention include but are not limited to: CD20, CD19, Her2, EGFR, EpCAM, CD3, FcγRIIIa (CD16), FcγRIIa (CD32a), FcγRIIb (CD32b), FcγRI (CD64), Toll-like receptors (TLRs) such as TLR4 and TLR9, cytokines such as IL-2, IL-5, IL-13, IL-12, IL-23, and TNFa, cytokine receptors such as IL-2R, chemokines, chemokine receptors, growth factors such as VEGF and HGF, and the like.

The choice of suitable target antigens depends on the desired therapeutic application. Some targets that have proven especially amenable to antibody therapy are those with signaling functions. Other therapeutic antibodies exert their effects by blocking signaling of the receptor by inhibiting the binding between a receptor and its cognate ligand. Another mechanism of action of therapeutic antibodies is to cause receptor down regulation. Other antibodies do not work by signaling through their target antigen. The choice of targets will depend on the detailed biology underlying the pathology of the indication that is being treated.

Monoclonal antibody therapy has emerged as an important therapeutic modality for cancer (Weiner et al., 2010, Nature Reviews Immunology 10:317-327; Reichert et al., 2005, Nature Biotechnology 23[9]:1073-1078; herein expressly incorporated by reference). For anti-cancer treatment it may be desirable to target an antigen whose expression is restricted to the cancerous cells or targeting an antigen that mediates some immunulogical killing activity. Exemplary targets for oncology include but are not limited to HGF and VEGF, IGF-1R and VEGF, Her2 and VEGF, CD19 and CD3, CD20 and CD3, Her2 and CD3, CD19 and FcγRIIIa, CD20 and FcγRIIIa, Her2 and FcγRIIIa. An immunoglobulin of the invention may be capable of binding VEGF and phosphatidylserine; VEGF and ErbB3; VEGF and PLGF; VEGF and ROBO4; VEGF and BSG2; VEGF and CDCP1; VEGF and ANPEP; VEGF and c-MET; HER-2 and ERB3; HER-2 and BSG2; HER-2 and CDCP1; HER-2 and ANPEP; EGFR and CD64; EGFR and BSG2; EGFR and CDCP1; EGFR and ANPEP; IGF1R and PDGFR; IGF1R and VEGF; IGF1R and CD20; CD20 and CD74; CD20 and CD30; CD20 and DR4; CD20 and VEGFR2; CD20 and CD52; CD20 and CD4; HGF and c-MET; HGF and NRP1; HGF and phosphatidylserine; ErbB3 and IGF1R; ErbB3 and IGF1,2; c-Met and Her-2; c-Met and NRP1; c-Met and IGF1R; IGF1,2 and PDGFR; IGF1,2 and CD20; IGF1,2 and IGF1R; IGF2 and EGFR; IGF2 and HER2; IGF2 and CD20; IGF2 and VEGF; IGF2 and IGF1R; IGF1 and IGF2; PDGFRa and VEGFR2; PDGFRa and PLGF; PDGFRa and VEGF; PDGFRa and c-Met; PDGFRa and EGFR; PDGFRb and VEGFR2; PDGFRb and c-Met; PDGFRb and EGFR; RON and c-Met; RON and MTSP1; RON and MSP; RON and CDCP1; VGFR1 and PLGF; VGFR1 and RON; VGFR1 and EGFR; VEGFR2 and PLGF; VEGFR2 and NRP1; VEGFR2 and RON; VEGFR2 and DLL4; VEGFR2 and EGFR; VEGFR2 and ROBO4; VEGFR2 and CD55; LPA and SIP; EPHB2 and RON; CTLA4 and VEGF; CD3 and EPCAM; CD40 and IL6; CD40 and IGF; CD40 and CD56; CD40 and CD70; CD40 and VEGFR1; CD40 and DR5; CD40 and DR4; CD40 and APRIL; CD40 and BCMA; CD40 and RANKL; CD28 and MAPG; CD80 and CD40; CD80 and CD30; CD80 and CD33; CD80 and CD74; CD80 and CD2; CD80 and CD3; CD80 and CD19; CD80 and CD4; CD80 and CD52; CD80 and VEGF; CD80 and DR5; CD80 and VEGFR2; CD22 and CD20; CD22 and CD80; CD22 and CD40; CD22 and CD23; CD22 and CD33; CD22 and CD74; CD22 and CD19; CD22 and DR5; CD22 and DR4; CD22 and VEGF; CD22 and CD52; CD30 and CD20; CD30 and CD22; CD30 and CD23; CD30 and CD40; CD30 and VEGF; CD30 and CD74; CD30 and CD19; CD30 and DR5; CD30 and DR4; CD30 and VEGFR2; CD30 and CD52; CD30 and CD4; CD138 and RANKL; CD33 and FTL3; CD33 and VEGF; CD33 and VEGFR2; CD33 and CD44; CD33 and DR4; CD33 and DR5; DR4 and CD137; DR4 and IGF1,2; DR4 and IGF1R; DR4 and DR5; DR5 and CD40; DR5 and CD137; DR5 and CD20; DR5 and EGFR; DR5 and IGF1,2; DR5 and IGFR, DR5 and HER-2, and EGFR and DLL4. Other target combinations include one or more members of the EGF/erb-2/erb-3 family.

Other targets (one or more) involved in oncological diseases that the immunoglobulins herein may bind include, but are not limited to those selected from the group consisting of: CD52, CD20, CD19, CD3, CD4, CD8, BMP6, IL12A, IL1A, IL1B, 1L2, IL24, INHA, TNF, TNFSF10, BMP6, EGF, FGF1, FGF10, FGF11, FGF12, FGF13, FGF14, FGF16, FGF17, FGF18, FGF19, FGF2, FGF20, FGF21, FGF22, FGF23, FGF3, FGF4, FGF5, FGF6, FGF7, FGF8, FGF9, GRP, IGF1, IGF2, IL12A, IL1A, IL1B, 1L2, INHA, TGFA, TGFB1, TGFB2, TGFB3, VEGF, CDK2, FGF10, FGF18, FGF2, FGF4, FGF7, IGF1R, IL2, BCL2, CD164, CDKN1A, CDKN1B, CDKN1C, CDKN2A, CDKN2B, CDKN2C, CDKN3, GNRH1, IGFBP6, IL1A, IL1B, ODZ1, PAWR, PLG, TGFB1I1, AR, BRCA1, CDK3, CDK4, CDK5, CDK6, CDK7, CDK9, E2F1, EGFR, ENO1, ERBB2, ESR1, ESR2, IGFBP3, IGFBP6, IL2, INSL4, MYC, NOX5, NR6A1, PAP, PCNA, PRKCQ, PRKD1, PRL, TP53, FGF22, FGF23, FGF9, IGFBP3, IL2, INHA, KLK6, TP53, CHGB, GNRH1, IGF1, IGF2, INHA, INSL3, INSL4, PRL, KLK6, SHBG, NR1D1, NR1H3, NR1I3, NR2F6, NR4A3, ESR1, ESR2, NROB1, NROB2, NR1D2, NR1H2, NR1H4, NR112, NR2C1, NR2C2, NR2E1, NR2E3, NR2F1, NR2F2, NR3C1, NR3C2, NR4A1, NR4A2, NR5A1, NR5A2, NR6 PGR, BARB, FGF1, FGF2, FGF6, KLK3, KRT1, APOC1, BRCA1, CHGA, CHGB, CLU, COL1A1, COL6A1, EGF, ERBB2, ERK8, FGF1, FGF10, FGF11, FGF13, FGF14, FGF16, FGF17, FGF18, FGF2, FGF20, FGF21, FGF22, FGF23, FGF3, FGF4, FGF5, FGF6, FGF7, FGF8, FGF9, GNRH1, IGF1, IGF2, IGFBP3, IGFBP6, IL12A, IL1A, IL1B, 1L2, IL24, INHA, INSL3, INSL4, KLK10, KLK12, KLK13, KLK14, KLK15, KLK3, KLK4, KLK5, KLK6, KLK9, MMP2, MMP9, MSMB, NTN4, ODZ1, PAP, PLAU, PRL, PSAP, SERPINA3, SHBG, TGFA, TIMP3, CD44, CDH1, CDH10, CDH19, CDH20, CDH7, CDH9, CDH1, CDH10, CDH13, CDH18, CDH19, CDH20, CDH7, CDH8, CDH9, ROBO2, CD44, ILK, ITGA1, APC, CD164, COL6A1, MTSS1, PAP, TGFB1I1, AGR2, AIG1, AKAP1, AKAP2, CANT1, CAV1, CDH12, CLDN3, CL/V3, CYB5, CYC1, DAB21P, DES, DNCL1, ELAC2, ENO2, ENO3, FASN, FLJ12584, FLJ25530, GAGEB1, GAGEC1, GGT1, GSTP1, HIP1, HUMCYT2A, IL29, K6HF, KAI1, KRT2A, MIB1, PART1, PATE, PCA3, PIAS2, PIK3CG, PPID, PR1, PSCA, SLC2A2, SLC33 SLC43 STEAP, STEAP2, TPM1, TPM2, TRPC6, ANGPT1, ANGPT2, ANPEP, ECGF1, EREG, FGF1, FGF2, FIGF, FLT1, JAG1, KDR, LAMAS, NRP1, NRP2, PGF, PLXDC1, STAB 1, VEGF, VEGFC, ANGPTL3, BAIL COL4A3, IL8, LAMAS, NRP1, NRP2, STAB 1, ANGPTL4, PECAM1, PF4, PROK2, SERPINF1, TNFAIP2, CCL11, CCL2, CXCL1, CXCL10, CXCL3, CXCL5, CXCL6, CXCL9, IFNA1, IFNB1, IFNG, IL1B, IL6, MDK, EDG1, EFNA1, EFNA3, EFNB2, EGF, EPHB4, FGFR3, HGF, IGF1, ITGB3, PDGFA, TEK, TGFA, TGFB1, TGFB2, TGFBR1, CCL2, CDH5, COL1A1, EDG1, ENG, ITGAV, ITGB3, THBS1, THBS2, BAD, BAG1, BCL2, CCNA1, CCNA2, CCND1, CCNE1, CCNE2, CDH1 (E-cadherin), CDKN1B (p27Kip1), CDKN2A (p161NK4a), COL6A1, CTNNB1 (b-catenin), CTSB (cathepsin B), ERBB2 (Her-2), ESR1, ESR2, F3 (TF), FOSL1 (FRA-1), GATA3, GSN (Gelsolin), IGFBP2, IL2RA, IL6, IL6R, IL6ST (glycoprotein 130), ITGA6 (a6 integrin), JUN, KLK5, KRT19, MAP2K7 (c-Jun), MKI67 (Ki-67), NGFB (GF), NGFR, NME1 (M23A), PGR, PLAU (uPA), PTEN, SERPINBS (maspin), SERPINE1 (PAI-1), TGFA, THBS1 (thrombospondin-1), TIE (Tie-1), TNFRSF6 (Fas), TNFSF6 (FasL), TOP2A (topoisomerase Iia), TP53, AZGP1 (zinc-a-glycoprotein), BPAG1 (plectin), CDKN1A (p21Wap1/Cip1), CLDN7 (claudin-7), CLU (clusterin), ERBB2 (Her-2), FGF1, FLRT1 (fibronectin), GABRP (GABAa), GNAS1, ID2, ITGA6 (a6 integrin), ITGB4 (b 4 integrin), KLF5 (GC Box BP), KRT19 (Keratin 19), KRTHB6 (hair-specific type II keratin), MACMARCKS, MT3 (metallothionectin-III), MUC1 (mucin), PTGS2 (COX-2), RAC2 (p21Rac2), S100A2, SCGB1D2 (lipophilin B), SCGB2A1 (mammaglobin 2), SCGB2A2 (mammaglobin 1), SPRR1B (Spr1), THBS1, THBS2, THBS4, and TNFAIP2 (B94), RON, c-Met, CD64, DLL4, PLGF, CTLA4, phophatidylserine, ROBO4, CD80, CD22, CD40, CD23, CD28, CD80, CD55, CD38, CD70, CD74, CD30, CD138, CD56, CD33, CD2, CD137, DR4, DR5, RANKL, VEGFR2, PDGFR, VEGFR1, MTSP1, MSP, EPHB2, EPHA1, EPHA2, EpCAM, PGE2, NKG2D, LPA, SIP, APRIL, BCMA, MAPG, FLT3, PDGFR alpha, PDGFR beta, ROR1, PSMA, PSCA, SCD1, and CD59.

Monoclonal antibody therapy has become an important therapeutic modality for treating autoimmune and inflammatory disorders (Chan & Carter, 2010, Nature Reviews Immunology 10:301-316; Reichert et al., 2005, Nature Biotechnology 23[9]:1073-1078; herein expressly incorporated by reference). Many proteins have been implicated in general autoimmune and inflammatory responses, and thus may be targeted by the immunogloublins of the invention. Autoimmune and inflammatory targets include but are not limited to C5, CCL1 (1-309), CCL11 (eotaxin), CCL13 (mcp-4), CCL15 (MIP-1d), CCL16 (HCC-4), CCL17 (TARC), CCL18 (PARC), CCL19, CCL2 (mcp-1), CCL20 (MIP-3a), CCL21 (MIP-2), CCL23 (MPIF-1), CCL24 (MPIF-2/eotaxin-2), CCL25 (TECK), CCL26, CCL3 (MIP-1a), CCL4 (MIP-1b), CCL5 (RANTES), CCL7 (mcp-3), CCL8 (mcp-2), CXCL1, CXCL10 (IP-10), CXCL11 (1-TAC/IP-9), CXCL12 (SDF1), CXCL13, CXCL14, CXCL2, CXCL3, CXCL5 (ENA-78/LIX), CXCL6 (GCP-2), CXCL9, IL13, IL8, CCL13 (mcp-4), CCR1, CCR2, CCR3, CCR4, CCR5, CCR6, CCR7, CCR8, CCR9, CX3CR1, IL8RA, XCR1 (CCXCR1), IFNA2, IL10, IL13, IL17C, IL1A, IL1B, IL1F10, IL1F5, IL1F6, IL1F7, IL1F8, IL1F9, IL22, IL5, IL8, IL9, LTA, LTB, MIF, SCYE1 (endothelial Monocyte-activating cytokine), SPP1, TNF, TNFSF5, IFNA2, IL10RA, IL10RB, IL13, IL13RA1, IL5RA, IL9, IL9R, ABCF1, BCL6, C3, C4A, CEBPB, CRP, ICEBERG, IL1R1, IL1RN, IL8RB, LTB4R, TOLLIP, FADD, IRAK1, IRAK2, MYD88, NCK2, TNFAIP3, TRADD, TRAF1, TRAF2, TRAF3, TRAF4, TRAF5, TRAF6, ACVR1, ACVR1B, ACVR2, ACVR2B, ACVRL1, CD28, CD3E, CD3G, CD3Z, CD69, CD80, CD86, CNR1, CTLA4, CYSLTR1, FCER1A, FCER2, FCGR3A, GPR44, HAVCR2, OPRD1, P2RX7, TLR2, TLR3, TLR4, TLR5, TLR6, TLR7, TLR8, TLR9, TLR10, BLR1, CCL1, CCL2, CCL3, CCL4, CCL5, CCL7, CCL8, CCL11, CCL13, CCL15, CCL16, CCL17, CCL18, CCL19, CCL20, CCL21, CCL22, CCL23, CCL24, CCL25, CCR1, CCR2, CCR3, CCR4, CCR5, CCR6, CCR7, CCR8, CCR9, CX3CL1, CX3CR1, CXCL1, CXCL2, CXCL3, CXCL5, CXCL6, CXCL10, CXCL11, CXCL12, CXCL13, CXCR4, GPR2, SCYE1, SDF2, XCL1, XCL2, XCR1, AMH, AMHR2, BMPR1A, BMPR1B, BMPR2, C19orf10 (IL27w), CER1, CSF1, CSF2, CSF3, DKFZp451J0118, FGF2, GFI1, IFNA1, IFNB1, IFNG, IGF1, IL1A, IL1B, IL1R1, IL1R2, IL2, IL2RA, IL2RB, IL2RG, IL3, IL4, IL4R, IL5, IL5RA, IL6, IL6R, IL6ST, IL7, IL8, IL8RA, IL8RB, IL9, IL9R, IL10, IL10RA, IL10RB, IL11, IL12RA, IL12A, IL12B, IL12RB1, IL12RB2, IL13, IL13RA1, IL13RA2, IL15, IL15RA, IL16, IL17, IL17R, IL18, IL18R1, IL19, IL20, KITLG, LEP, LTA, LTB, LTB4R, LTB4R2, LTBR, MIF, NPPB, PDGFB, TBX21, TDGF1, TGFA, TGFB1, TGFB1I1, TGFB2, TGFB3, TGFB1, TGFBR1, TGFBR2, TGFBR3, TH1L, TNF, TNFRSF1A, TNFRSF1B, TNFRSF7, TNFRSF8, TNFRSF9, TNFRSF11A, TNFRSF21, TNFSF4, TNFSF5, TNFSF6, TNFSF1I, VEGF, ZFPM2, and RNF110 (ZNF144).

Targets that the immunoglobulins described herein can bind and be useful to treat asthma may be determined. In an embodiment, such targets include, but are not limited to, CSF1 (MCSF), CSF2 (GM-CSF), CSF3 (GCSF), FGF2, IFNA1, IFNB1, IFNG, histamine and histamine receptors, IL1A, IL1B, IL2, IL3, IL4, IL5, IL6, IL7, IL8, IL9, IL10, IL11, IL12A, IL12B, IL13, IL14, IL15, IL16, IL17, IL18, IL19, KITLG, PDGFB, IL2RA, IL4R, IL5RA, IL8RA, IL8RB, IL12RB1, IL12RB2, IL13RA1, IL13RA2, IL18R1, TSLP, CCLi, CCL2, CCL3, CCL4, CCL5, CCL7, CCL8, CCL13, CCL17, CCL18, CCL19, CCL20, CCL22, CCL24,CX3CL1, CXCL1, CXCL2, CXCL3, XCLi, CCR2, CCR3, CCR4, CCR5, CCR6, CCR7, CCR8, CX3CR1, GPR2, XCR1, FOS, GATA3, JAK1, JAK3, STAT6, TBX21, TGFB1, TNF, TNFSF6, YY1, CYSLTR1, FCER1A, FCER2, LTB4R, TB4R2, LTBR, and Chitinase.

Targets involved in rheumatoid arthritis (RA) include but are not limited to TNF, IL-18, IL-12, IL-23, IL-1beta, MIF, IL-17, and IL-15.

Antigens that may be targeted in order to treat systemic lupus erythematosus (SLE) by the immunoglobulins herein include but are not limited to CD-20, CD-22, CD-19, CD28, CD4, CD80, HLA-DRA, IL10, IL2, IL4, TNFRSF5, TNFRSF6, TNFSF5, TNFSF6, BLR1, HDAC4, HDAC5, HDAC7A, HDAC9, ICOSL, IGBP1, MS4A1, RGSI, SLA2, CD81, IFNB1, IL10, TNFRSF5, TNFRSF7, TNFSF5, AICDA, BLNK, GALNAC4S-6ST, HDAC4, HDAC5, HDAC7A, HDAC9, IL10, IL11, IL4, INHA, INHBA, KLF6, TNFRSF7, CD28, CD38, CD69, CD80, CD83, CD86, DPP4, FCER2, IL2RA, TNFRSF8, TNFSF7, CD24, CD37, CD40, CD72, CD74, CD79A, CD79B, CR2, ILIR2, ITGA2, ITGA3, MS4A1, ST6GALI, CDIC, CHSTIO, HLA-A, HLA-DRA, and NT5E.; CTLA4, B7.1, B7.2, BlyS, BAFF, C5, IL-4, IL-6, IL-10, IFN-α, and TNF-α.

The immunoglobulins herein may target antigens for the treatment of multiple sclerosis (MS), including but not limited to IL-12, TWEAK, IL-23, CXCL13, CD40, CD40L, IL-18, VEGF, VLA-4, TNF, CD45RB, CD200, IFNgamma, GM-CSF, FGF, C5, CD52, and CCR2. An embodiment includes co-engagement of anti-IL-12 and TWEAK for the treatment of MS.

One aspect of the invention pertains to immunoglobulins capable of binding a target involved in sepsis, e.g., selected from the group consisting TNF, IL-1, MIF, IL-6, IL-8, IL-18, IL-12, IL-23, FasL, LPS, Toll-like receptors, TLR-4, tissue factor, MIP-2, ADORA2A, CASP1, CASP4, IL-10, IL-1B, NFκB1, PROC, TNFRSFIA, CSF3, CCR3, ILIRN, MIF, NFκB1, PTAFR, TLR2, TLR4, GPR44, HMOX1, midkine, IRAK1, NFκB2, SERPINA1, SERPINE1, and TREM1.

In some cases, immunoglobulins herein may be directed against antigens for the treatment of infectious diseases.

### Antibodies For Engineering

A number of antibodies and Fc fusions that are approved for use, in clinical trials or in development, may benefit from the Fc variants of the present invention. A list of exemplary antibodies and Fc fusions that may benefit from the Fc modifications described herein is provided in Tables 1-3 below: K

TABLE 1

| International non-proprietary | Manufacturing | | | First EU (US) | |
|---|---|---|---|---|---|
| name (Trade name) | cell line | Type | Target | approval year | |
| Abciximab (reopro ®) | sp2/0 | Chimeric igG1KFab | GPiib/iiia | 1995* | (1994) |
| rituximab (Mabthera ®, rituxan ®) | CHo | Chimeric igG1K | Cd20 | 1998 | (1997) |
| Basiliximab (simulect ®) | sp2/0 | Chimeric igG1K | iL2r | 1998 | (1998) |
| Palivizumab (synagis ®) | ns0 | Humanized igG1K | rsV | 1999 | (1998) |
| infliximab (remicade ®) | sp2/0 | Chimeric igG1K | tnF | 1999 | (1998) |
| trastuzumab (Herceptin ®) | CHo | Humanized igG1K | HEr2 | 2000 | (1998) |
| Alemtuzumab (MabCampath, Campath-1H ®) | CHo | Humanized igG1K | Cd52 | 2001 | (2001) |
| Adalimumab (Humira ®) | CHo | Human igG1K | tnF | 2003 | (2002) |
| tositumomab-i131 (Bexxar ®) | Hybridoma | Murine igG2A | Cd20 | nA | (2003) |
| Cetuximab (Erbitux ®) | sp2/0 | Chimeric igG1K | EGFr | 2004 | (2004) |
| ibritumomab tiuxetan (Zevalin ®) | CHo | Murine igG1K | Cd20 | 2004 | (2002) |
| omalizumab (Xolair ®) | CHo | Humanized igG1K | igE | 2005 | (2003) |
| Bevacizumab (Avastin ®) | CHo | Humanized igG1K | VEGF | 2005 | (2004) |
| natalizumab (tysabri ®) | ns0 | Humanized igG4K | α4-integrin | 2006 | (2004) |
| ranibizumab (Lucentis ®) | E. coli | Humanized igG1KFab | VEGF | 2007 | (2006) |
| Panitumumab (Vectibix ®) | CHo | Human igG2K | EGFr | 2007 | (2006) |
| Eculizumab (soliris ®) | ns0 | Humanized igG2/4K | C5 | 2007 | (2007) |
| Certolizumab pegol (Cimzia ®) | E. coli | Humanized igG1K Fab, | tnF | 2009 | (2008) |
| | | pegylated | | | |
| Golimumab (simponi ®) | sp2/0 | Human igG1K | tnF | 2009 | (2009) |
| Canakinumab (ilaris ®) | sp2/0 | Human igG1K | iL1b | 2009 | (2009) |
| Catumaxomab (removab ®) | Hybrid hybridoma | rat igG2b/mouse igG2a | EpCAM/Cd3 | 2009 | (nA) |
| | | bispecific | | | |
| Ustekinumab (stelara ®) | sp2/0 | Human igG1K | iL12/23 | 2009 | (2009) |
| tocilizumab (roActemra, Actemra ®) | CHo | Humanized igG1K | iL6r | 2009 | (2010) |
| ofatumumab (Arzerra ®) | ns0 | Human igG1K | Cd20 | 2010 | (2009) |
| denosumab (Prolia ®) | CHo | Human igG2K | rAnK-L | 2010 | (2010) |
| Belimumab (Benlysta ®) | ns0 | Human igG1l | BLys | 2011 | (2011) |
| raxibacumab (Pending) | ns0** | Human igG1K | B. anthrasis | nA | (2012) |
| ipilimumab (Yervoy ®) | CHo | Human igG1K | CtLA-4 | 2011 | (2011) |
| Brentuximab vedotin (Adcentris ®) | CHo | ChimericigG1K; conjugated to | Cd30 | in review | (2011) |
| | | monomethyl auristatin E | | | |
| Pertuzumab (Perjeta ®) | CHo | Humanized igG1K | HEr2 | in review | (2012) |

*Country-specific approval; approved under concertation procedure
**Product manufactured for Phase 1 study in humans.
Abbreviations: BLys, B lymphocyte stimulator; C5, complement 5; Cd, cluster of differentiation; CHo, Chinese hamster ovary; CtLA-4, cytotoxic t lymphocyte antigen 4; EGFr, epidermal growth factor receptor; EpCAM, epithelial cell adhesion molecule; Fab, antigen-binding fragment; GP glycoprotein; iL, interleukin; nA, not approved; PA, protective antigen; rAnK-L, receptor activator of nFKb ligand; rsV, respiratory syncy-tial virus; tnF, tumor necrosis factor; VEGF, vascular endothelial growth factor.
sources: European Medicines Agency public assessment reports, United states Food and drug Administration (drugs@fda), the international imMunoGenetics information system ® (www.imgt.org/mAb-dB/index).

TABLE 2

| International non-proprietary | Manufacturing | | | First EU (US) | |
|---|---|---|---|---|---|
| name (Trade name) | cell line | Type | Target | approval year | |
| Muromonab-Cd3 (orthoclone oKt3 ®) | Hybridoma | Murine igG2a | Cd3 | 1986* | (1986) |
| nebacumab (Centoxin ®) | Hybridoma | Human igM | Endotoxin | 1991* | (nA) |
| Edrecolomab (Panorex ®) | Hybridoma | Murine igG2a | EpCAM | 1995* | (nA) |
| daclizumab (Zenapax ®) | ns0 | Humanized igG1κ | iL2r | 1999 | (1997) |
| Gemtuzumab ozogamicin (Mylotarg ®) | ns0 | Humanized igG4κ | Cd33 | nA | (2000) |
| Efalizumab (raptiva ®) | CHo | Humanized igG1κ | Cd11a | 2004 | (2003) |

*European country-specific approval.
Abbreviations: Cd, cluster of differentiation; CHo, Chinese hamster ovary; EpCAM, epithelial cell adhesion molecule; iL, interleukin; nA, not approved.
sources: European Medicines Agency public assessment reports, United states Food and drug Administration (drugs@fda), the international imMunoGenetics information system ® (www.imgt.org/mAb-dB/index).

TABLE 3

| International proprietary | Manufacturing | | | First appproval |
|---|---|---|---|---|
| name (Trade name) | cell line | Type | Target | year |
| nimotuzumab (thera CiM ®, BioMAB-EGF ® | ns0 | Humanized igG1κ | EGFr | 1999 |
| Mogamulizumab (Poteligeo ®) | [not found] | Humanized igG1κ | CCr4 | 2012 |

Abbreviations: CCr, chemokine receptor; EGFr, epidermal growth factor receptor.

These antibodies are herein referred to as “clinical products and candidates”. Thus, in a preferred embodiment, the Fc variants of the present invention may find use in a range of clinical products and candidates. For example, the Fc variants of the present invention may find use in an antibody that has components, e.g., the variable domains, the CDRs, etc., of clinical antibodies including, but not limited to, rituximab (Rituxan®, IDEC/Genentech/Roche) (see, for example U.S. Pat. No. 5,736,137), a chimeric anti-CD20 antibody approved to treat Non-Hodgkin's lymphoma; HuMax-CD20, an anti-CD20 currently being developed by Genmab, an anti-CD20 antibody described in U.S. Pat. No. 5,500,362, AME-133 (Applied Molecular Evolution), hA20 (Immunomedics, Inc.), HumaLYM (Intracel), and PRO70769 (PCT/US2003/040426, entitled “Immunoglobulin Variants and Uses Thereof”). A number of antibodies that target members of the family of epidermal growth factor receptors, including EGFR (ErbB-1), Her2/neu (ErbB-2), Her3 (ErbB-3), Her4 (ErbB-4), may benefit from Fc modifications of the present invention. For example, the Fc variants of the present invention may find use in an antibody that is substantially similar to trastuzumab (Herceptin®, Genentech) (see, for example, U.S. Pat. No. 5,677,171), a humanized anti-Her2/neu antibody approved to treat breast cancer; pertuzumab (rhuMab-2C4, Omnitarg™), currently being developed by Genentech; an anti-Her2 antibody described in U.S. Pat. No. 4,753,894; cetuximab (Erbitux®, Imclone) (U.S. Pat. No. 4,943,533; PCT WO 96/40210), a chimeric anti-EGFR antibody in clinical trials for a variety of cancers; ABX-EGF (U.S. Pat. No. 6,235,883), currently being developed by Abgenix-Immunex-Amgen; HuMax-EGFr (U.S. Ser. No. 10/172,317), currently being developed by Genmab; 425, EMD55900, EMD62000, and EMD72000 (Merck KGaA) (U.S. Pat. No. 5,558,864; Murthy et al. 1987, Arch Biochem Biophys. 252(2):549-60; Rodeck et al., 1987, J Cell Biochem. 35(4):315-20; Kettleborough et al., 1991, Protein Eng. 4(7):773-83); ICR62 (Institute of Cancer Research) (PCT WO 95/20045; Modjtahedi et al., 1993, J. Cell Biophys. 1993, 22(1-3):129-46; Modjtahedi et al., 1993, Br J Cancer. 1993, 67(2):247-53; Modjtahedi et al., 1996, Br J Cancer, 73(2):228-35; Modjtahedi et al., 2003, Int J Cancer, 105(2):273-80); TheraCIM hR3 (YM Biosciences, Canada and Centro de Immunologia Molecular, Cuba (U.S. Pat. No. 5,891,996; U.S. Pat. No. 6,506,883; Mateo et al., 1997, Immunotechnology, 3(1):71-81); mAb-806 (Ludwig Institue for Cancer Research, Memorial Sloan-Kettering) (Jungbluth et al. 2003, Proc Natl Acad Sci USA. 100(2):639-44); KSB-102 (KS Biomedix); MR1-1 (IVAX, National Cancer Institute) (PCT WO 0162931A2); and SC100 (Scancell) (PCT WO 01/88138). In another preferred embodiment, the Fc variants of the present invention may find use in alemtuzumab (Campath®, Millenium), a humanized monoclonal antibody currently approved for treatment of B-cell chronic lymphocytic leukemia.

The Fc polypeptides of the present invention may find use in a variety of antibodies that are substantially similar to other clinical products and candidates, including but not limited to muromonab-CD3 (Orthoclone OKT3®), an anti-CD3 antibody developed by Ortho Biotech/Johnson & Johnson, ibritumomab tiuxetan (Zevalin®), an anti-CD20 antibody developed by IDEC/Schering AG, gemtuzumab ozogamicin (Mylotarg®), an anti-CD33 (p67 protein) antibody developed by Celltech/Wyeth, alefacept (Amevive®), an anti-LFA-3 Fc fusion developed by Biogen), abciximab (ReoPro®), developed by Centocor/Lilly, basiliximab (Simulect®), developed by Novartis, palivizumab (Synagis®), developed by Medlmmune, infliximab (Remicade®), an anti-TNFalpha antibody developed by Centocor, adalimumab (Humira®), an anti-TNFalpha antibody developed by Abbott, Humicade™, an anti-TNFalpha antibody developed by Celltech, etanercept (Enbrel®), an anti-TNFalpha Fc fusion developed by Immunex/Amgen, ABX-CBL, an anti-CD147 antibody being developed by Abgenix, ABX-IL8, an anti-IL8 antibody being developed by Abgenix, ABX-MA1, an anti-MUC18 antibody being developed by Abgenix, Pemtumomab (R1549, 90Y-muHMFG1), an anti-MUC1 In development by Antisoma, Therex (R1550), an anti-MUC1 antibody being developed by Antisoma, AngioMab (AS1405), being developed by Antisoma, HuBC-1, being developed by Antisoma, Thioplatin (AS1407) being developed by Antisoma, Antegren® (natalizumab), an anti-alpha-4-beta-1 (VLA-4) and alpha-4-beta-7 antibody being developed by Biogen, VLA-1 mAb, an anti-VLA-1 integrin antibody being developed by Biogen, LTBR mAb, an anti-lymphotoxin beta receptor (LTBR) antibody being developed by Biogen, CAT-152, an anti-TGF-β2 antibody being developed by Cambridge Antibody Technology, J695, an anti-IL-12 antibody being developed by Cambridge Antibody Technology and Abbott, CAT-192, an anti-TGFβ1 antibody being developed by Cambridge Antibody Technology and Genzyme, CAT-213, an anti-Eotaxin1 antibody being developed by Cambridge Antibody Technology, LymphoStat-BTM an anti-Blys antibody being developed by Cambridge Antibody Technology and Human Genome Sciences Inc., TRAIL-R1mAb, an anti-TRAIL-R1 antibody being developed by Cambridge Antibody Technology and Human Genome Sciences, Inc., Avastin™ (bevacizumab, rhuMAb-VEGF), an anti-VEGF antibody being developed by Genentech, an anti-HER receptor family antibody being developed by Genentech, Anti-Tissue Factor (ATF), an anti-Tissue Factor antibody being developed by Genentech, Xolair™ (Omalizumab), an anti-IgE antibody being developed by Genentech, Raptiva™ (Efalizumab), an anti-CD11a antibody being developed by Genentech and Xoma, MLN-02 Antibody (formerly LDP-02), being developed by Genentech and Millenium Pharmaceuticals, HuMax CD4, an anti-CD4 antibody being developed by Genmab, HuMax-IL15, an anti-IL15 antibody being developed by Genmab and Amgen, HuMax-Inflam, being developed by Genmab and Medarex, HuMax-Cancer, an anti-Heparanase I antibody being developed by Genmab and Medarex and Oxford GcoSciences, HuMax-Lymphoma, being developed by Genmab and Amgen, HuMax-TAC, being developed by Genmab, IDEC-131, and anti-CD40L antibody being developed by IDEC Pharmaceuticals, IDEC-151 (Clenoliximab), an anti-CD4 antibody being developed by IDEC Pharmaceuticals, IDEC-114, an anti-CD80 antibody being developed by IDEC Pharmaceuticals, IDEC-152, an anti-CD23 being developed by IDEC Pharmaceuticals, anti-macrophage migration factor (MIF) antibodies being developed by IDEC Pharmaceuticals, BEC2, an anti-idiotypic antibody being developed by Imclone, IMC-1C11, an anti-KDR antibody being developed by Imclone, DC101, an anti-flk-1 antibody being developed by Imclone, anti-VE cadherin antibodies being developed by Imclone, CEA-Cide™ (labetuzumab), an anti-carcinoembryonic antigen (CEA) antibody being developed by Immunomedics, LymphoCide™ (Epratuzumab), an anti-CD22 antibody being developed by Immunomedics, AFP-Cide, being developed by Immunomedics, MyelomaCide, being developed by Immunomedics, LkoCide, being developed by Immunomedics, ProstaCide, being developed by Immunomedics, MDX-010, an anti-CTLA4 antibody being developed by Medarex, MDX-060, an anti-CD30 antibody being developed by Medarex, MDX-070 being developed by Medarex, MDX-018 being developed by Medarex, Osidem™ (IDM-1), and anti-Her2 antibody being developed by Medarex and Immuno-Designed Molecules, HuMax™-CD4, an anti-CD4 antibody being developed by Medarex and Genmab, HuMax-IL15, an anti-IL15 antibody being developed by Medarex and Genmab, CNTO 148, an anti-TNFα antibody being developed by Medarex and Centocor/J&J, CNTO 1275, an anti-cytokine antibody being developed by Centocor/J&J, MOR101 and MOR102, anti-intercellular adhesion molecule-1 (ICAM-1) (CD54) antibodies being developed by MorphoSys, MOR201, an anti-fibroblast growth factor receptor 3 (FGFR-3) antibody being developed by MorphoSys, Nuvion® (visilizumab), an anti-CD3 antibody being developed by Protein Design Labs, HuZAF™, an anti-gamma interferon antibody being developed by Protein Design Labs, Anti-α5β1 Integrin, being developed by Protein Design Labs, anti-IL-12, being developed by Protein Design Labs, ING-1, an anti-Ep-CAM antibody being developed by Xoma, and MLN01, an anti-Beta2 integrin antibody being developed by Xoma; all of the above-cited references in this paragraph are expressly incorporated herein by reference.

The antibodies of the present invention are generally isolated or recombinant. “Isolated”, when used to describe the various polypeptides disclosed herein, means a polypeptide that has been identified and separated and/or recovered from a cell or cell culture from which it was expressed. Ordinarily, an isolated polypeptide will be prepared by at least one purification step. An “isolated antibody,” refers to an antibody which is substantially free of other antibodies having different antigenic specificities.

“Specific binding” or “specifically binds to” or is “specific for” a particular antigen or an epitope means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target.

Specific binding for a particular antigen or an epitope can be exhibited, for example, by an antibody having a KD for an antigen or epitope of at least about 10-4 M, at least about 10-5 M, at least about 10-6 M, at least about 10-7 M, at least about 10-8 M, at least about 10-9 M, alternatively at least about 10-10 M, at least about 10-11 M, at least about 10-12 M, or greater, where KD refers to a dissociation rate of a particular antibody-antigen interaction. Typically, an antibody that specifically binds an antigen will have a KD that is 20-, 50-, 100-, 500-, 1000-, 5,000-, 10,000- or more times greater for a control molecule relative to the antigen or epitope.

Also, specific binding for a particular antigen or an epitope can be exhibited, for example, by an antibody having a KA or Ka for an antigen or epitope of at least 20-, 50-, 100-, 500-, 1000-, 5,000-, 10,000- or more times greater for the epitope relative to a control, where KA or Ka refers to an association rate of a particular antibody-antigen interaction.

### Antibody-Drug Conjugates

In some embodiments, the Fc polypeptides of the invention are conjugated with drugs to form antibody-drug conjugates (ADCs). In general, ADCs are used in oncology applications, where the use of antibody-drug conjugates for the local delivery of cytotoxic or cytostatic agents allows for the targeted delivery of the drug moiety to tumors, which can allow higher efficacy, lower toxicity, etc. An overview of this technology is provided in Ducry et al., Bioconjugate Chem., 21:5-13 (2010), Carter et al., Cancer J. 14(3):154 (2008) and Senter, Current Opin. Chem. Biol. 13:235-244 (2009), all of which are hereby incorporated by reference in their entirety.

Thus, the invention provides Fc polypeptides conjugated to drugs. Generally, conjugation is done by covalent attachment to the antibody, as further described below, and generally relies on a linker, often a peptide linkage (which, as described below, may be designed to be sensitive to cleavage by proteases at the target site or not). In addition, as described above, linkage of the linker-drug unit (LU-D) can be done by attachment to cysteines within the antibody. As will be appreciated by those in the art, the number of drug moieties per antibody can change, depending on the conditions of the reaction, and can vary from 1:1 to 10:1 drug:antibody. As will be appreciated by those in the art, the actual number is an average.

Thus, the invention provides Fc polypeptides conjugated to drugs. As described below, the drug of the ADC can be any number of agents, including but not limited to cytotoxic agents such as chemotherapeutic agents, growth inhibitory agents, toxins (for example, an enzymatically active toxin of bacterial, fungal, plant, or animal origin, or fragments thereof), or a radioactive isotope (that is, a radioconjugate) are provided. In other embodiments, the invention further provides methods of using the ADCs.

Drugs for use in the present invention include cytotoxic drugs, particularly those which are used for cancer therapy. Such drugs include, in general, DNA damaging agents, anti-metabolites, natural products and their analogs. Exemplary classes of cytotoxic agents include the enzyme inhibitors such as dihydrofolate reductase inhibitors, and thymidylate synthase inhibitors, DNA intercalators, DNA cleavers, topoisomerase inhibitors, the anthracycline family of drugs, the vinca drugs, the mitomycins, the bleomycins, the cytotoxic nucleosides, the pteridine family of drugs, diynenes, the podophyllotoxins, dolastatins, maytansinoids, differentiation inducers, and taxols.

Members of these classes include, for example, methotrexate, methopterin, dichloromethotrexate, 5-fluorouracil, 6-mercaptopurine, cytosine arabinoside, melphalan, leurosine, leurosideine, actinomycin, daunorubicin, doxorubicin, mitomycin C, mitomycin A, caminomycin, aminopterin, tallysomycin, podophyllotoxin and podophyllotoxin derivatives such as etoposide or etoposide phosphate, vinblastine, vincristine, vindesine, taxanes including taxol, taxotere retinoic acid, butyric acid, N8-acetyl spermidine, camptothecin, calicheamicin, esperamicin, ene-diynes, duocarmycin A, duocarmycin SA, calicheamicin, camptothecin, maytansinoids (including DM1), monomethylauristatin E (MMAE), monomethylauristatin F (MMAF), and maytansinoids (DM4) and their analogues.

Toxins may be used as antibody-toxin conjugates and include bacterial toxins such as diphtheria toxin, plant toxins such as ricin, small molecule toxins such as geldanamycin (Mandler et al., (2000) J. Nat. Cancer Inst. 92(19):1573-1581; Mandler et al., 2000, Bioorganic & Med. Chem. Letters 10:1025-1028; Mandler et al., 2002, Bioconjugate Chem. 13:786-791), maytansinoids (EP 1391213; Liu et al., 1996, Proc. Natl. Acad. Sci. USA 93:8618-8623), and calicheamicin (Lode et al., 1998, Cancer Res. 58:2928; Hinman et al., 1993, Cancer Res. 53:3336-3342). Toxins may exert their cytotoxic and cytostatic effects by mechanisms including tubulin binding, DNA binding, or topoisomerase inhibition.

Conjugates of a Fc polypeptide of the invention and one or more small molecule toxins, such as a maytansinoids, dolastatins, auristatins, a trichothecene, calicheamicin, and CC1065, and the derivatives of these toxins that have toxin activity, are contemplated.

### Maytansinoids

Maytansine compounds suitable for use as maytansinoid drug moieties are well known in the art, and can be isolated from natural sources according to known methods, produced using genetic engineering techniques (see Yu et al., 2002, PNAS 99:7968-7973), or maytansinol and maytansinol analogues prepared synthetically according to known methods. As described below, drugs may be modified by the incorporation of a functionally active group such as a thiol or amine group for conjugation to the antibody.

Exemplary maytansinoid drug moieties include those having a modified aromatic ring, such as: C-19-dechloro (U.S. Pat. No. 4,256,746) (prepared by lithium aluminum hydride reduction of ansamytocin P2); C-20-hydroxy (or C-20-demethyl)+/−C-19-dechloro (U.S. Pat. Nos. 4,361,650 and 4,307,016) (prepared by demethylation using Streptomyces or Actinomyces or dechlorination using LAH); and C-20-demethoxy, C-20-acyloxy (—OCOR), +/−dechloro (U.S. Pat. No. 4,294,757) (prepared by acylation using acyl chlorides) and those having modifications at other positions.

Exemplary maytansinoid drug moieties also include those having modifications such as: C-9-SH (U.S. Pat. No. 4,424,219) (prepared by the reaction of maytansinol with H2S or P2S5); C-14-alkoxymethyl (demethoxy/CH2OR) (U.S. Pat. No. 4,331,598); C-14-hydroxymethyl or acyloxymethyl (CH2OH or CH2OAc) (U.S. Pat. No. 4,450,254) (prepared from Nocardia); C-15-hydroxy/acyloxy (U.S. Pat. No. 4,364,866) (prepared by the conversion of maytansinol by Streptomyces); C-15-methoxy (U.S. Pat. Nos. 4,313,946 and 4,315,929) (isolated from Trewia nudlflora); C-18-N-demethyl (U.S. Pat. Nos. 4,362,663 and 4,322,348) (prepared by the demethylation of maytansinol by Streptomyces); and 4,5-deoxy (U.S. Pat. No. 4,371,533) (prepared by the titanium trichloride/LAH reduction of maytansinol).

Of particular use are DM1 (disclosed in U.S. Pat. No. 5,208,020, incorporated by reference) and DM4 (disclosed in U.S. Pat. No. 7,276,497, incorporated by reference). See also a number of additional maytansinoid derivatives and methods in 5,416,064, WO/01/24763, 7,303,749, 7,601,354, U.S. Ser. No. 12/631,508, WO02/098883, 6,441,163, 7,368,565, WO02/16368 and WO04/1033272, all of which are expressly incorporated by reference in their entirety.

ADCs containing maytansinoids, methods of making same, and their therapeutic use are disclosed, for example, in U.S. Pat. Nos. 5,208,020; 5,416,064; 6,441,163 and European Patent EP 0 425 235 B1, the disclosures of which are hereby expressly incorporated by reference. Liu et al., Proc. Natl. Acad. Sci. USA 93:8618-8623 (1996) described ADCs comprising a maytansinoid designated DM1 linked to the monoclonal antibody C242 directed against human colorectal cancer. The conjugate was found to be highly cytotoxic towards cultured colon cancer cells, and showed antitumor activity in an in vivo tumor growth assay.

Chari et al., Cancer Research 52:127-131 (1992) described ADCs in which a maytansinoid was conjugated via a disulfide linker to the murine antibody A7 binding to an antigen on human colon cancer cell lines, or to another murine monoclonal antibody TA.1 that binds the HER-2/neu oncogene. The cytotoxicity of the TA.1-maytansonoid conjugate was tested in vitro on the human breast cancer cell line SK-BR-3, which expresses 3×105 HER-2 surface antigens per cell. The drug conjugate achieved a degree of cytotoxicity similar to the free maytansinoid drug, which could be increased by increasing the number of maytansinoid molecules per antibody molecule. The A7-maytansinoid conjugate showed low systemic cytotoxicity in mice.

### Auristatins and Dolastatins

In some embodiments, the ADC comprises a Fc polypeptide conjugated to dolastatins or dolostatin peptidic analogs and derivatives, the auristatins (U.S. Pat. Nos. 5,635,483; 5,780,588). Dolastatins and auristatins have been shown to interfere with microtubule dynamics, GTP hydrolysis, and nuclear and cellular division (Woyke et al., 2001, Antimicrob. Agents and Chemother. 45(12):3580-3584) and have anticancer (U.S. Pat. No. 5,663,149) and antifungal activity (Pettit et al., 1998, Antimicrob. Agents Chemother. 42:2961-2965). The dolastatin or auristatin drug moiety may be attached to the antibody through the N (amino) terminus or the C (carboxyl) terminus of the peptidic drug moiety (WO 02/088172).

Exemplary auristatin embodiments include the N-terminus linked monomethylauristatin drug moieties DE and DF, disclosed in “Senter et al., Proceedings of the American Association for Cancer Research, Volume 45, Abstract Number 623, presented Mar. 28, 2004 and described in United States Patent Publication No. 2005/0238648, the disclosure of which is expressly incorporated by reference in its entirety.

An exemplary auristatin embodiment is MMAE (see U.S. Pat. No. 6,884,869 expressly incorporated by reference in its entirety).

Another exemplary auristatin embodiment is MMAF (see US 2005/0238649, 5,767,237 and 6,124,431, expressly incorporated by reference in their entirety).

Additional exemplary embodiments comprising MMAE or MMAF and various linker components (described further herein) have the following structures and abbreviations (wherein Ab means antibody and p is 1 to about 8):

Typically, peptide-based drug moieties can be prepared by forming a peptide bond between two or more amino acids and/or peptide fragments. Such peptide bonds can be prepared, for example, according to the liquid phase synthesis method (see E. Schroder and K. Lubke, “The Peptides”, volume 1, pp 76-136, 1965, Academic Press) that is well known in the field of peptide chemistry. The auristatin/dolastatin drug moieties may be prepared according to the methods of: U.S. Pat. No. 5,635,483; U.S. Pat. No. 5,780,588; Pettit et al., 1989, J. Am. Chem. Soc. 111:5463-5465; Pettit et al., 1998, Anti-Cancer Drug Design 13:243-277; Pettit, G. R., et al., Synthesis, 1996, 719-725; Pettit et al., 1996, J. Chem. Soc. Perkin Trans. 1 5:859-863; and Doronina (2003) Nat Biotechnol 21(7):778-784.

### Calicheamicin

In other embodiments, the ADC comprises an antibody of the invention conjugated to one or more calicheamicin molecules. For example, Mylotarg is the first commercial ADC drug and utilizes calicheamicin γ1 as the payload (see U.S. Pat. No. 4,970,198, incorporated by reference in its entirety). Additional calicheamicin derivatives are described in U.S. Pat. Nos. 5,264,586, 5,384,412, 5,550,246, 5,739,116, 5,773,001, 5,767,285 and 5,877,296, all expressly incorporated by reference. The calicheamicin family of antibiotics are capable of producing double-stranded DNA breaks at sub-picomolar concentrations. For the preparation of conjugates of the calicheamicin family, see U.S. Pat. Nos. 5,712,374, 5,714,586, 5,739,116, 5,767,285, 5,770,701, 5,770,710, 5,773,001, 5,877,296 (all to American Cyanamid Company). Structural analogues of calicheamicin which may be used include, but are not limited to, γ1I, α2I, α2I, N-acetyl- γ1I, PSAG and θI1 (Hinman et al., Cancer Research 53:3336-3342 (1993), Lode et al., Cancer Research 58:2925-2928 (1998) and the aforementioned U.S. patents to American Cyanamid). Another anti-tumor drug that the antibody can be conjugated is QFA which is an antifolate. Both calicheamicin and QFA have intracellular sites of action and do not readily cross the plasma membrane. Therefore, cellular uptake of these agents through antibody mediated internalization greatly enhances their cytotoxic effects.

### Duocarmycins

CC-1065 (see 4,169,888, incorporated by reference) and duocarmycins are members of a family of antitumor antibiotics utilized in ADCs. These antibiotics appear to work through sequence-selectively alkylating DNA at the N3 of adenine in the minor groove, which initiates a cascade of events that result in apoptosis.

Important members of the duocarmycins include duocarmycin A (U.S. Pat. No. 4,923,990, incorporated by reference) and duocarmycin SA (U.S. Pat. No. 5,101,038, incorporated by reference), and a large number of analogues as described in U.S. Pat. Nos. 7,517,903, 7,691,962, 5,101,038; 5,641,780; 5,187,186; 5,070,092; 5,641,780; 5,101,038; 5,084,468, 5,475,092, 5,585,499, 5,846,545, WO2007/089149, WO2009/017394A1, 5,703,080, 6,989,452, 7,087,600, 7,129,261, 7,498,302, and 7,507,420, all of which are expressly incorporated by reference.

### Other Cytotoxic Agents

Other antitumor agents that can be conjugated to the antibodies of the invention include BCNU, streptozoicin, vincristine and 5-fluorouracil, the family of agents known collectively LL-E33288 complex described in U.S. Pat. Nos. 5,053,394, 5,770,710, as well as esperamicins (U.S. Pat. No. 5,877,296).

Enzymatically active toxins and fragments thereof which can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin and the tricothecenes. See, for example, WO 93/21232 published Oct. 28, 1993.

The present invention further contemplates an ADC formed between an antibody and a compound with nucleolytic activity (e.g., a ribonuclease or a DNA endonuclease such as a deoxyribonuclease; DNase).

For selective destruction of the tumor, the antibody may comprise a highly radioactive atom. A variety of radioactive isotopes are available for the production of radioconjugated antibodies. Examples include At211, I131, I125, Y90, Re186, Re188, Sm153, Bi212, P32, Pb212 and radioactive isotopes of Lu.

The radio- or other labels may be incorporated in the conjugate in known ways. For example, the peptide may be biosynthesized or may be synthesized by chemical amino acid synthesis using suitable amino acid precursors involving, for example, fluorine-19 in place of hydrogen. Labels such as Tc99m or I123, Re186, Re188 and In111 can be attached via a cysteine residue in the peptide. Yttrium-90 can be attached via a lysine residue. The IODOGEN method (Fraker et al., 1978, Biochem. Biophys. Res. Commun. 80: 49-57 can be used to incorporate Iodine-123. “Monoclonal Antibodies in Immunoscintigraphy” (Chatal, CRC Press 1989) describes other methods in detail.

For compositions comprising a plurality of antibodies, the drug loading is represented by p, the average number of drug molecules per Antibody. Drug loading may range from 1 to 20 drugs (D) per Antibody. The average number of drugs per antibody in preparation of conjugation reactions may be characterized by conventional means such as mass spectroscopy, ELISA assay, and HPLC. The quantitative distribution of Antibody-Drug-Conjugates in terms of p may also be determined.

In some instances, separation, purification, and characterization of homogeneous Antibody-Drug-conjugates where p is a certain value from Antibody-Drug-Conjugates with other drug loadings may be achieved by means such as reverse phase HPLC or electrophoresis. In exemplary embodiments, p is 2, 3, 4, 5, 6, 7, or 8 or a fraction thereof

The generation of Antibody-drug conjugate compounds can be accomplished by any technique known to the skilled artisan. Briefly, the Antibody-drug conjugate compounds can include a Fc polypeptide, as the Antibody unit, a drug, and optionally a linker that joins the drug and the binding agent.

A number of different reactions are available for covalent attachment of drugs and/or linkers to binding agents. This is can be accomplished by reaction of the amino acid residues of the binding agent, for example, antibody molecule, including the amine groups of lysine, the free carboxylic acid groups of glutamic and aspartic acid, the sulfhydryl groups of cysteine and the various moieties of the aromatic amino acids. A commonly used non-specific methods of covalent attachment is the carbodiimide reaction to link a carboxy (or amino) group of a compound to amino (or carboxy) groups of the antibody. Additionally, bifunctional agents such as dialdehydes or imidoesters have been used to link the amino group of a compound to amino groups of an antibody molecule.

Also available for attachment of drugs to binding agents is the Schiff base reaction. This method involves the periodate oxidation of a drug that contains glycol or hydroxy groups, thus forming an aldehyde which is then reacted with the binding agent. Attachment occurs via formation of a Schiff base with amino groups of the binding agent. Isothiocyanates can also be used as coupling agents for covalently attaching drugs to binding agents. Other techniques are known to the skilled artisan and within the scope of the present invention.

In some embodiments, an intermediate, which is the precursor of the linker, is reacted with the drug under appropriate conditions. In other embodiments, reactive groups are used on the drug and/or the intermediate. The product of the reaction between the drug and the intermediate, or the derivatized drug, is subsequently reacted with a Fc variant of the invention under appropriate conditions.

It will be understood that chemical modifications may also be made to the desired compound in order to make reactions of that compound more convenient for purposes of preparing conjugates of the invention. For example a functional group e.g., amine, hydroxyl, or sulfhydryl, may be appended to the drug at a position which has minimal or an acceptable effect on the activity or other properties of the drug.

### Linker Units

Typically, the antibody-drug conjugate compounds comprise a linker unit between the drug unit and the antibody unit. In some embodiments, the linker is cleavable under intracellular or extracellular conditions, such that cleavage of the linker releases the drug unit from the antibody in the appropriate environment. For example, solid tumors that secrete certain proteases may serve as the target of the cleavable linker; in other embodiments, it is the intracellular proteases that are utilized. In yet other embodiments, the linker unit is not cleavable and the drug is released, for example, by antibody degradation in lysosomes.

In some embodiments, the linker is cleavable by a cleaving agent that is present in the intracellular environment (for example, within a lysosome or endosome or caveolea). The linker can be, for example, a peptidyl linker that is cleaved by an intracellular peptidase or protease enzyme, including, but not limited to, a lysosomal or endosomal protease. In some embodiments, the peptidyl linker is at least two amino acids long or at least three amino acids long or more.

Cleaving agents can include, without limitation, cathepsins B and D and plasmin, all of which are known to hydrolyze dipeptide drug derivatives resulting in the release of active drug inside target cells (see, e.g., Dubowchik and Walker, 1999, Pharm. Therapeutics 83:67-123). Peptidyl linkers that are cleavable by enzymes that are present in CD38-expressing cells. For example, a peptidyl linker that is cleavable by the thiol-dependent protease cathepsin-B, which is highly expressed in cancerous tissue, can be used (e.g., a Phe-Leu or a Gly-Phe-Leu-Gly linker (SEQ ID NO: 112)). Other examples of such linkers are described, e.g., in U.S. Pat. No. 6,214,345, incorporated herein by reference in its entirety and for all purposes.

In some embodiments, the peptidyl linker cleavable by an intracellular protease is a Val-Cit linker or a Phe-Lys linker (see, e.g., U.S. Pat. No. 6,214,345, which describes the synthesis of doxorubicin with the val-cit linker).

In other embodiments, the cleavable linker is pH-sensitive, that is, sensitive to hydrolysis at certain pH values. Typically, the pH-sensitive linker hydrolyzable under acidic conditions. For example, an acid-labile linker that is hydrolyzable in the lysosome (for example, a hydrazone, semicarbazone, thiosemicarbazone, cis-aconitic amide, orthoester, acetal, ketal, or the like) may be used. (See, e.g., U.S. Pat. Nos. 5,122,368; 5,824,805; 5,622,929; Dubowchik and Walker, 1999, Pharm. Therapeutics 83:67-123; Neville et al., 1989, Biol. Chem. 264:14653-14661.) Such linkers are relatively stable under neutral pH conditions, such as those in the blood, but are unstable at below pH 5.5 or 5.0, the approximate pH of the lysosome. In certain embodiments, the hydrolyzable linker is a thioether linker (such as, e.g., a thioether attached to the therapeutic agent via an acylhydrazone bond (see, e.g., U.S. Pat. No. 5,622,929).

In yet other embodiments, the linker is cleavable under reducing conditions (for example, a disulfide linker). A variety of disulfide linkers are known in the art, including, for example, those that can be formed using SATA (N-succinimidyl-5-acetylthioacetate), SPDP (N-succinimidyl-3-(2-pyridyldithio)propionate), SPDB (N-succinimidyl-3-(2-pyridyldithio)butyrate) and SMPT (N-succinimidyl-oxycarbonyl-alpha-methyl-alpha-(2-pyridyl-dithio)toluene), SPDB and SMPT. (See, e.g., Thorpe et al., 1987, Cancer Res. 47:5924-5931; Wawrzynczak et al., In Immunoconjugates: Antibody Conjugates in Radioimagery and Therapy of Cancer (C. W. Vogel ed., Oxford U. Press, 1987. See also U.S. Pat. No. 4,880,935.)

In other embodiments, the linker is a malonate linker (Johnson et al., 1995, Anticancer Res. 15:1387-93), a maleimidobenzoyl linker (Lau et al., 1995, Bioorg-Med-Chem. 3(10):1299-1304), or a 3′-N-amide analog (Lau et al., 1995, Bioorg-Med-Chem. 3(10):1305-12).

In yet other embodiments, the linker unit is not cleavable and the drug is released by antibody degradation. (See U.S. Publication No. 2005/0238649 incorporated by reference herein in its entirety and for all purposes).

In many embodiments, the linker is self-immolative. As used herein, the term “self-immolative Spacer” refers to a bifunctional chemical moiety that is capable of covalently linking together two spaced chemical moieties into a stable tripartite molecule. It will spontaneously separate from the second chemical moiety if its bond to the first moiety is cleaved. See for example, WO 2007059404A2, WO06110476A2, WO05112919A2, WO2010/062171, WO09/017394, WO07/089149, WO 07/018431, WO04/043493 and WO02/083180, which are directed to drug-cleavable substrate conjugates where the drug and cleavable substrate are optionally linked through a self-immolative linker and which are all expressly incorporated by reference.

Often the linker is not substantially sensitive to the extracellular environment. As used herein, “not substantially sensitive to the extracellular environment,” in the context of a linker, means that no more than about 20%, 15%, 10%, 5%, 3%, or no more than about 1% of the linkers, in a sample of antibody-drug conjugate compound, are cleaved when the antibody-drug conjugate compound presents in an extracellular environment (for example, in plasma).

Whether a linker is not substantially sensitive to the extracellular environment can be determined, for example, by incubating with plasma the antibody-drug conjugate compound for a predetermined time period (for example, 2, 4, 8, 16, or 24 hours) and then quantitating the amount of free drug present in the plasma.

In other, non-mutually exclusive embodiments, the linker promotes cellular internalization. In certain embodiments, the linker promotes cellular internalization when conjugated to the therapeutic agent (that is, in the milieu of the linker-therapeutic agent moiety of the antibody-drug conjugate compound as described herein). In yet other embodiments, the linker promotes cellular internalization when conjugated to both the auristatin compound and the Fc variants of the invention.

A variety of exemplary linkers that can be used with the present compositions and methods are described in WO 2004-010957, U.S. Publication No. 2006/0074008, U.S. Publication No. 20050238649, and U.S. Publication No. 2006/0024317 (each of which is incorporated by reference herein in its entirety and for all purposes).

### Drug Loading

Drug loading is represented by p and is the average number of Drug moieties per antibody in a molecule. Drug loading (“p”) may be 1, 2, 3, 4, 5, 6, 7, 8, 9, 10, 11, 12, 13, 14, 15, 16, 17, 18, 19, 20 or more moieties (D) per antibody, although frequently the average number is a fraction or a decimal. Generally, drug loading of from 1 to 4 is frequently useful, and from 1 to 2 is also useful. ADCs of the invention include collections of antibodies conjugated with a range of drug moieties, from 1 to 20. The average number of drug moieties per antibody in preparations of ADC from conjugation reactions may be characterized by conventional means such as mass spectroscopy and, ELISA assay.

The quantitative distribution of ADC in terms of p may also be determined. In some instances, separation, purification, and characterization of homogeneous ADC where p is a certain value from ADC with other drug loadings may be achieved by means such as electrophoresis.

For some antibody-drug conjugates, p may be limited by the number of attachment sites on the antibody. For example, where the attachment is a cysteine thiol, as in the exemplary embodiments above, an antibody may have only one or several cysteine thiol groups, or may have only one or several sufficiently reactive thiol groups through which a linker may be attached. In certain embodiments, higher drug loading, e.g., p>5, may cause aggregation, insolubility, toxicity, or loss of cellular permeability of certain antibody-drug conjugates. In certain embodiments, the drug loading for an ADC of the invention ranges from 1 to about 8; from about 2 to about 6; from about 3 to about 5; from about 3 to about 4; from about 3.1 to about 3.9; from about 3.2 to about 3.8; from about 3.2 to about 3.7; from about 3.2 to about 3.6; from about 3.3 to about 3.8; or from about 3.3 to about 3.7. Indeed, it has been shown that for certain ADCs, the optimal ratio of drug moieties per antibody may be less than 8, and may be about 2 to about 5. See US 2005-0238649 A1 (herein incorporated by reference in its entirety).

In certain embodiments, fewer than the theoretical maximum of drug moieties are conjugated to an antibody during a conjugation reaction. An antibody may contain, for example, lysine residues that do not react with the drug-linker intermediate or linker reagent, as discussed below. Generally, antibodies do not contain many free and reactive cysteine thiol groups which may be linked to a drug moiety; indeed most cysteine thiol residues in antibodies exist as disulfide bridges. In certain embodiments, an antibody may be reduced with a reducing agent such as dithiothreitol (DTT) or tricarbonylethylphosphine (TCEP), under partial or total reducing conditions, to generate reactive cysteine thiol groups. In certain embodiments, an antibody is subjected to denaturing conditions to reveal reactive nucleophilic groups such as lysine or cysteine.

The loading (drug/antibody ratio) of an ADC may be controlled in different ways, e.g., by: (i) limiting the molar excess of drug-linker intermediate or linker reagent relative to antibody, (ii) limiting the conjugation reaction time or temperature, (iii) partial or limiting reductive conditions for cysteine thiol modification, (iv) engineering by recombinant techniques the amino acid sequence of the antibody such that the number and position of cysteine residues is modified for control of the number and/or position of linker-drug attachments (such as thioMab or thioFab prepared as disclosed herein and in WO2006/034488 (herein incorporated by reference in its entirety)).

It is to be understood that where more than one nucleophilic group reacts with a drug-linker intermediate or linker reagent followed by drug moiety reagent, then the resulting product is a mixture of ADC compounds with a distribution of one or more drug moieties attached to an antibody. The average number of drugs per antibody may be calculated from the mixture by a dual ELISA antibody assay, which is specific for antibody and specific for the drug. Individual ADC molecules may be identified in the mixture by mass spectroscopy and separated by HPLC, e.g., hydrophobic interaction chromatography.

In some embodiments, a homogeneous ADC with a single loading value may be isolated from the conjugation mixture by electrophoresis or chromatography.

### Methods of Determining Cytotoxic Effect of ADCs

Methods of determining whether a Drug or Antibody-Drug conjugate exerts a cytostatic and/or cytotoxic effect on a cell are known. Generally, the cytotoxic or cytostatic activity of an Antibody Drug conjugate can be measured by: exposing mammalian cells expressing a target protein of the Antibody Drug conjugate in a cell culture medium; culturing the cells for a period from about 6 hours to about 5 days; and measuring cell viability. Cell-based in vitro assays can be used to measure viability (proliferation), cytotoxicity, and induction of apoptosis (caspase activation) of the Antibody Drug conjugate.

For determining whether an Antibody Drug conjugate exerts a cytostatic effect, a thymidine incorporation assay may be used. For example, cancer cells expressing a target antigen at a density of 5,000 cells/well of a 96-well plated can be cultured for a 72-hour period and exposed to 0.5 μCi of 3H-thymidine during the final 8 hours of the 72-hour period. The incorporation of 3H-thymidine into cells of the culture is measured in the presence and absence of the Antibody Drug conjugate.

For determining cytotoxicity, necrosis or apoptosis (programmed cell death) can be measured. Necrosis is typically accompanied by increased permeability of the plasma membrane; swelling of the cell, and rupture of the plasma membrane. Apoptosis is typically characterized by membrane blebbing, condensation of cytoplasm, and the activation of endogenous endonucleases. Determination of any of these effects on cancer cells indicates that an Antibody Drug conjugate is useful in the treatment of cancers.

Cell viability can be measured by determining in a cell the uptake of a dye such as neutral red, trypan blue, or ALAMAR™ blue (see, e.g., Page et al., 1993, Intl. J. Oncology 3:473-476). In such an assay, the cells are incubated in media containing the dye, the cells are washed, and the remaining dye, reflecting cellular uptake of the dye, is measured spectrophotometrically. The protein-binding dye sulforhodamine B (SRB) can also be used to measure cytoxicity (Skehan et al., 1990, J. Natl. Cancer Inst. 82:1107-12).

Alternatively, a tetrazolium salt, such as MTT, is used in a quantitative colorimetric assay for mammalian cell survival and proliferation by detecting living, but not dead, cells (see, e.g., Mosmann, 1983, J. Immunol. Methods 65:55-63).

Apoptosis can be quantitated by measuring, for example, DNA fragmentation. Commercial photometric methods for the quantitative in vitro determination of DNA fragmentation are available. Examples of such assays, including TUNEL (which detects incorporation of labeled nucleotides in fragmented DNA) and ELISA-based assays, are described in Biochemica, 1999, no. 2, pp. 34-37 (Roche Molecular Biochemicals).

Apoptosis can also be determined by measuring morphological changes in a cell. For example, as with necrosis, loss of plasma membrane integrity can be determined by measuring uptake of certain dyes (e.g., a fluorescent dye such as, for example, acridine orange or ethidium bromide). A method for measuring apoptotic cell number has been described by Duke and Cohen, Current Protocols in Immunology (Coligan et al. eds., 1992, pp. 3.17.1-3.17.16). Cells also can be labeled with a DNA dye (e.g., acridine orange, ethidium bromide, or propidium iodide) and the cells observed for chromatin condensation and margination along the inner nuclear membrane. Other morphological changes that can be measured to determine apoptosis include, e.g., cytoplasmic condensation, increased membrane blebbing, and cellular shrinkage.

The presence of apoptotic cells can be measured in both the attached and “floating” compartments of the cultures. For example, both compartments can be collected by removing the supernatant, trypsinizing the attached cells, combining the preparations following a centrifugation wash step (e.g., 10 minutes at 2000 rpm), and detecting apoptosis (e.g., by measuring DNA fragmentation). (See, e.g., Piazza et al., 1995, Cancer Research 55:3110-16).

In vivo, the effect of a therapeutic composition of the antibodies of the invention can be evaluated in a suitable animal model. For example, xenogenic cancer models can be used, wherein cancer explants or passaged xenograft tissues are introduced into immune compromised animals, such as nude or SCID mice (Klein et al., 1997, Nature Medicine 3: 402-408). Efficacy can be measured using assays that measure inhibition of tumor formation, tumor regression or metastasis, and the like.

Engineering IgG Variants

The IgG variants can be based on human IgG sequences, and thus human IgG sequences are used as the “base” sequences against which other sequences are compared, including but not limited to sequences from other organisms, for example rodent and primate sequences. IgG variants may also comprise sequences from other immunoglobulin classes such as IgA, IgE, IgGD, IgGM, and the like. It is contemplated that, although the IgG variants are engineered in the context of one parent IgG, the variants may be engineered in or “transferred” to the context of another, second parent IgG. This is done by determining the “equivalent” or “corresponding” residues and substitutions between the first and second IgG, typically based on sequence or structural homology between the sequences of the two IgGs. In order to establish homology, the amino acid sequence of a first IgG outlined herein is directly compared to the sequence of a second IgG. After aligning the sequences, using one or more of the homology alignment programs known in the art (for example, using conserved residues as between species), allowing for necessary insertions and deletions in order to maintain alignment (i.e., avoiding the elimination of conserved residues through arbitrary deletion and insertion), the residues equivalent to particular amino acids in the primary sequence of the first IgG variant are defined. Alignment of conserved residues preferably should conserve 100% of such residues. However, alignment of greater than 75% or as little as 50% of conserved residues is also adequate to define equivalent residues. Equivalent residues may also be defined by determining structural homology between a first and second IgG that is at the level of tertiary structure for IgGs whose structures have been determined. In this case, equivalent residues are defined as those for which the atomic coordinates of two or more of the main chain atoms of a particular amino acid residue of the parent or precursor (N on N, CA on CA, C on C and O on O) are within 0.13 nm and preferably 0.1 nm after alignment. Alignment is achieved after the best model has been oriented and positioned to give the maximum overlap of atomic coordinates of non-hydrogen protein atoms of the proteins. Regardless of how equivalent or corresponding residues are determined, and regardless of the identity of the parent IgG in which the IgGs are made, what is meant to be conveyed is that the IgG variants discovered by can be engineered into any second parent IgG that has significant sequence or structural homology with the IgG variant. Thus, for example, if a variant antibody is generated wherein the parent antibody is human IgG1, by using the methods described above or other methods for determining equivalent residues, the variant antibody may be engineered in another IgG1 parent antibody that binds a different antigen, a human IgG2 parent antibody, a human IgA parent antibody, a mouse IgG2a or IgG2b parent antibody, and the like. Again, as described above, the context of the parent IgG variant does not affect the ability to transfer the IgG variants to other parent IgGs.

Methods for engineering, producing, and screening IgG variants are provided. The described methods are not meant to constrain to any particular application or theory of operation. Rather, the provided methods are meant to illustrate generally that one or more IgG variants may be engineered, produced, and screened experimentally to obtain IgG variants with optimized effector function. A variety of methods are described for designing, producing, and testing antibody and protein variants in U.S. Ser. No. 10/754,296, and U.S. Ser. No. 10/672,280, which are herein expressly incorporated by reference.

A variety of protein engineering methods may be used to design IgG variants with optimized effector function. In one embodiment, a structure-based engineering method may be used, wherein available structural information is used to guide substitutions. In a preferred embodiment, a computational screening method may be used, wherein substitutions are designed based on their energetic fitness in computational calculations. See, for example, U.S. Ser. No. 10/754,296 and U.S. Ser. No. 10/672,280, and references cited therein. By “computational screening method” herein is meant any method for designing one or more mutations in a protein, wherein said method utilizes a computer to evaluate the energies of the interactions of potential amino acid side chain substitutions with each other and/or with the rest of the protein. As will be appreciated by those skilled in the art, evaluation of energies, referred to as energy calculation, refers to some method of scoring one or more amino acid modifications. Said method may involve a physical or chemical energy term, or may involve knowledge-, statistical-, sequence-based energy terms, and the like. The calculations that compose a computational screening method are herein referred to as “computational screening calculations”.

An alignment of sequences may be used to guide substitutions at the identified positions. One skilled in the art will appreciate that the use of sequence information may curb the introduction of substitutions that are potentially deleterious to protein structure. The source of the sequences may vary widely, and include one or more of the known databases, including but not limited to the Kabat database (Northwestern University); Johnson & Wu, 2001, Nucleic Acids Res. 29:205-206; Johnson & Wu, 2000, Nucleic Acids Res. 28:214-218), the IMGT database (IMGT, the international ImMunoGeneTics Information System®; Lefranc et al., 1999, Nucleic Acids Res. 27:209-212; Ruiz et al., 2000 Nucleic Acids Res. 28:219-221; Lefranc et al., 2001, Nucleic Acids Res. 29:207-209; Lefranc et al., 2003, Nucleic Acids Res. 31:307-310), and VBASE. Antibody sequence information can be obtained, compiled, and/or generated from sequence alignments of germline sequences or sequences of naturally occurring antibodies from any organism, including but not limited to mammals. One skilled in the art will appreciate that the use of sequences that are human or substantially human may further have the advantage of being less immunogenic when administered to a human. Other databases which are more general nucleic acid or protein databases, i.e., not particular to antibodies, include but are not limited to SwissProt, GenBank Entrez, and EMBL Nucleotide Sequence Database. Aligned sequences can include VH, VL, CH, and/or CL sequences. There are numerous sequence-based alignment programs and methods known in the art, and all of these find use in for generation of sequence alignments.

Alternatively, random or semi-random mutagenesis methods may be used to make amino acid modifications at the desired positions. In these cases positions are chosen randomly, or amino acid changes are made using simplistic rules. For example, all residues may be mutated to alanine, referred to as alanine scanning. Such methods may be coupled with more sophisticated engineering approaches that employ selection methods to screen higher levels of sequence diversity. As is well known in the art, there are a variety of selection technologies that may be used for such approaches, including, for example, display technologies such as phage display, ribosome display, cell surface display, and the like, as described below.

Methods for production and screening of IgG variants are well known in the art. General methods for antibody molecular biology, expression, purification, and screening are described in Antibody Engineering, edited by Duebel & Kontermann, Springer-Verlag, Heidelberg, 2001; and Hayhurst & Georgiou, 2001, Curr Opin Chem Biol 5:683-689; Maynard & Georgiou, 2000, Annu Rev Biomed Eng 2:339-76. Also, see the methods described in U.S. Ser. No. 10/754,296, filed on Mar. 3, 2003, U.S. Ser. No. 10/672,280, filed Sep. 29, 2003, and U.S. Ser. No. 10/822,231, filed Mar. 26, 2004.

Production of Fc Variants

The present invention provides methods for producing and experimentally testing Fc variants. The described methods are not meant to constrain the present invention to any particular application or theory of operation. Rather, the provided methods are meant to illustrate generally that one or more Fc variants may be produced and experimentally tested to obtain Fc variants. General methods for antibody molecular biology, expression, purification, and screening are described in Antibody Engineering, edited by Duebel & Kontermann, Springer-Verlag, Heidelberg, 2001; and Hayhurst & Georgiou, 2001, Curr Opin Chem Biol 5:683-689; Maynard & Georgiou, 2000, Annu Rev Biomed Eng 2:339-76; Antibodies: A Laboratory Manual by Harlow & Lane, New York: Cold Spring Harbor Laboratory Press, 1988, all incorporated entirely by reference.

In one embodiment of the present invention, nucleic acids are created that encode the Fc variants, and that may then be cloned into host cells, expressed and assayed, if desired. Thus, nucleic acids, and particularly DNA, may be made that encode each protein sequence. These practices are carried out using well-known procedures. For example, a variety of methods that may find use in the present invention are described in Molecular Cloning—A Laboratory Manual, 3^{rd} Ed. (Maniatis, Cold Spring Harbor Laboratory Press, New York, 2001), and Current Protocols in Molecular Biology (John Wiley & Sons), both incorporated entirely by reference. As will be appreciated by those skilled in the art, the generation of exact sequences for a library comprising a large number of sequences is potentially expensive and time consuming. By “library” herein is meant a set of variants in any form, including but not limited to a list of nucleic acid or amino acid sequences, a list of nucleic acid or amino acid substitutions at variable positions, a physical library comprising nucleic acids that encode the library sequences, or a physical library comprising the variant proteins, either in purified or unpurified form. Accordingly, there are a variety of techniques that may be used to efficiently generate libraries of the present invention. Such methods that may find use in the present invention are described or referenced in U.S. Pat. No. 6,403,312; U.S. Ser. No. 09/782,004; U.S. Ser. No. 09/927,790; U.S. Ser. No. 10/218,102; PCT WO 01/40091; and PCT WO 02/25588, all incorporated entirely by reference. Such methods include but are not limited to gene assembly methods, PCR-based method and methods which use variations of PCR, ligase chain reaction-based methods, pooled oligo methods such as those used in synthetic shuffling, error-prone amplification methods and methods which use oligos with random mutations, classical site-directed mutagenesis methods, cassette mutagenesis, and other amplification and gene synthesis methods. As is known in the art, there are a variety of commercially available kits and methods for gene assembly, mutagenesis, vector subcloning, and the like, and such commercial products find use in the present invention for generating nucleic acids that encode Fc variants.

The Fc variants of the present invention may be produced by culturing a host cell transformed with nucleic acid, preferably an expression vector, containing nucleic acid encoding the Fc variants, under the appropriate conditions to induce or cause expression of the protein. The conditions appropriate for expression will vary with the choice of the expression vector and the host cell, and will be easily ascertained by one skilled in the art through routine experimentation. A wide variety of appropriate host cells may be used, including but not limited to mammalian cells, bacteria, insect cells, and yeast. For example, a variety of cell lines that may find use in the present invention are described in the ATCC® cell line catalog, available from the American Type Culture Collection.

In a preferred embodiment, the Fc variants are expressed in mammalian expression systems, including systems in which the expression constructs are introduced into the mammalian cells using virus such as retrovirus or adenovirus. Any mammalian cells may be used, with human, mouse, rat, hamster, and primate cells being particularly preferred. Suitable cells also include known research cells, including but not limited to Jurkat T cells, NIH3T3, CHO, BHK, COS, HEK293, PER C.6, HeLa, Sp2/0, NSO cells and variants thereof. In an alternately preferred embodiment, library proteins are expressed in bacterial cells. Bacterial expression systems are well known in the art, and include Escherichia coli (E. coli), Bacillus subtilis, Streptococcus cremoris, and Streptococcus lividans. In alternate embodiments, Fc variants are produced in insect cells (e.g., Sf21/Sf9, Trichoplusia ni Bti-Tn5b1-4) or yeast cells (e.g., S. cerevisiae, Pichia, etc.). In an alternate embodiment, Fc variants are expressed in vitro using cell free translation systems. In vitro translation systems derived from both prokaryotic (e.g., E. coli) and eukaryotic (e.g., wheat germ, rabbit reticulocytes) cells are available and may be chosen based on the expression levels and functional properties of the protein of interest. For example, as appreciated by those skilled in the art, in vitro translation is required for some display technologies, for example ribosome display. In addition, the Fc variants may be produced by chemical synthesis methods. Also transgenic expression systems both animal (e.g., cow, sheep or goat milk, embryonated hen's eggs, whole insect larvae, etc.) and plant (e.g., corn, tobacco, duckweed, etc.)

The nucleic acids that encode the Fc variants of the present invention may be incorporated into an expression vector in order to express the protein. A variety of expression vectors may be utilized for protein expression. Expression vectors may comprise self-replicating extra-chromosomal vectors or vectors which integrate into a host genome. Expression vectors are constructed to be compatible with the host cell type. Thus, expression vectors which find use in the present invention include but are not limited to those which enable protein expression in mammalian cells, bacteria, insect cells, yeast, and in in vitro systems. As is known in the art, a variety of expression vectors are available, commercially or otherwise, that may find use in the present invention for expressing Fc variants.

Expression vectors typically comprise a protein operably linked with control or regulatory sequences, selectable markers, any fusion partners, and/or additional elements. By “operably linked” herein is meant that the nucleic acid is placed into a functional relationship with another nucleic acid sequence. Generally, these expression vectors include transcriptional and translational regulatory nucleic acid operably linked to the nucleic acid encoding the Fc variant, and are typically appropriate to the host cell used to express the protein. In general, the transcriptional and translational regulatory sequences may include promoter sequences, ribosomal binding sites, transcriptional start and stop sequences, translational start and stop sequences, and enhancer or activator sequences. As is also known in the art, expression vectors typically contain a selection gene or marker to allow the selection of transformed host cells containing the expression vector. Selection genes are well known in the art and will vary with the host cell used.

Fc variants may be operably linked to a fusion partner to enable targeting of the expressed protein, purification, screening, display, and the like. Fusion partners may be linked to the Fc variant sequence via a linker sequences. The linker sequence will generally comprise a small number of amino acids, typically less than ten, although longer linkers may also be used. Typically, linker sequences are selected to be flexible and resistant to degradation. As will be appreciated by those skilled in the art, any of a wide variety of sequences may be used as linkers. For example, a common linker sequence comprises the amino acid sequence GGGGS. A fusion partner may be a targeting or signal sequence that directs Fc variant and any associated fusion partners to a desired cellular location or to the extracellular media. As is known in the art, certain signaling sequences may target a protein to be either secreted into the growth media, or into the periplasmic space, located between the inner and outer membrane of the cell. A fusion partner may also be a sequence that encodes a peptide or protein that enables purification and/or screening. Such fusion partners include but are not limited to polyhistidine tags (His-tags) (for example H₆ and H₁₀ or other tags for use with Immobilized Metal Affinity Chromatography (IMAC) systems (e.g., Ni⁺² affinity columns)), GST fusions, MBP fusions, Strep-tag, the BSP biotinylation target sequence of the bacterial enzyme BirA, and epitope tags which are targeted by antibodies (for example, c-myc tags, flag-tags, and the like). As will be appreciated by those skilled in the art, such tags may be useful for purification, for screening, or both. For example, an Fc variant may be purified using a His-tag by immobilizing it to a Ni+2 affinity column, and then after purification the same His-tag may be used to immobilize the antibody to a Ni′ coated plate to perform an ELISA or other binding assay (as described below). A fusion partner may enable the use of a selection method to screen Fc variants (see below). Fusion partners that enable a variety of selection methods are well-known in the art, and all of these find use in the present invention. For example, by fusing the members of an Fc variant library to the gene III protein, phage display can be employed (Kay et al., Phage display of peptides and proteins: a laboratory manual, Academic Press, San Diego, Calif., 1996; Lowman et al., 1991, Biochemistry 30:10832-10838; Smith, 1985, Science 228:1315-1317, incorporated entirely by reference). Fusion partners may enable Fc variants to be labeled. Alternatively, a fusion partner may bind to a specific sequence on the expression vector, enabling the fusion partner and associated Fc variant to be linked covalently or noncovalently with the nucleic acid that encodes them. The methods of introducing exogenous nucleic acid into host cells are well known in the art, and will vary with the host cell used. Techniques include but are not limited to dextran-mediated transfection, calcium phosphate precipitation, calcium chloride treatment, polybrene mediated transfection, protoplast fusion, electroporation, viral or phage infection, encapsulation of the polynucleotide(s) in liposomes, and direct microinjection of the DNA into nuclei. In the case of mammalian cells, transfection may be either transient or stable.

In a preferred embodiment, Fc variants are purified or isolated after expression. Proteins may be isolated or purified in a variety of ways known to those skilled in the art. Standard purification methods include chromatographic techniques, including ion exchange, hydrophobic interaction, affinity, sizing or gel filtration, and reversed-phase, carried out at atmospheric pressure or at high pressure using systems such as FPLC and HPLC. Purification methods also include electrophoretic, immunological, precipitation, dialysis, and chromatofocusing techniques. Ultrafiltration and diafiltration techniques, in conjunction with protein concentration, are also useful. As is well known in the art, a variety of natural proteins bind Fc and antibodies, and these proteins can find use in the present invention for purification of Fc variants. For example, the bacterial proteins A and G bind to the Fc region. Likewise, the bacterial protein L binds to the Fab region of some antibodies, as of course does the antibody's target antigen. Purification can often be enabled by a particular fusion partner. For example, Fc variants may be purified using glutathione resin if a GST fusion is employed, Ni⁺² affinity chromatography if a His-tag is employed, or immobilized anti-flag antibody if a flag-tag is used. For general guidance in suitable purification techniques, see, e.g., incorporated entirely by reference Protein Purification: Principles and Practice, 3rd Ed., Scopes, Springer-Verlag, NY, 1994, incorporated entirely by reference. The degree of purification necessary will vary depending on the screen or use of the Fc variants. In some instances no purification is necessary. For example, in one embodiment, if the Fc variants are secreted, screening may take place directly from the media. As is well known in the art, some methods of selection do not involve purification of proteins. Thus, for example, if a library of Fc variants is made into a phage display library, protein purification may not be performed.

In Vitro Experimentation

Fc variants may be screened using a variety of methods, including but not limited to those that use in vitro assays, in vivo and cell-based assays, and selection technologies. Automation and high-throughput screening technologies may be utilized in the screening procedures. Screening may employ the use of a fusion partner or label. The use of fusion partners has been discussed above. By “labeled” herein is meant that the Fc variants of the invention have one or more elements, isotopes, or chemical compounds attached to enable the detection in a screen. In general, labels fall into three classes: a) immune labels, which may be an epitope incorporated as a fusion partner that is recognized by an antibody, b) isotopic labels, which may be radioactive or heavy isotopes, and c) small molecule labels, which may include fluorescent and colorimetric dyes, or molecules such as biotin that enable other labeling methods. Labels may be incorporated into the compound at any position and may be incorporated in vitro or in vivo during protein expression.

In a preferred embodiment, the functional and/or biophysical properties of Fc variants are screened in an in vitro assay. In vitro assays may allow a broad dynamic range for screening properties of interest. Properties of Fc variants that may be screened include but are not limited to stability, solubility, and affinity for Fc ligands, for example FcγRs. Multiple properties may be screened simultaneously or individually. Proteins may be purified or unpurified, depending on the requirements of the assay. In one embodiment, the screen is a qualitative or quantitative binding assay for binding of Fc variants to a protein or nonprotein molecule that is known or thought to bind the Fc variant. In a preferred embodiment, the screen is a binding assay for measuring binding to the target antigen. In an alternately preferred embodiment, the screen is an assay for binding of Fc variants to an Fc ligand, including but are not limited to the family of FcγRs, the neonatal receptor FcRn, the complement protein C1q, and the bacterial proteins A and G. Said Fc ligands may be from any organism, with humans, mice, rats, rabbits, and monkeys preferred. Binding assays can be carried out using a variety of methods known in the art, including but not limited to FRET (Fluorescence Resonance Energy Transfer) and BRET (Bioluminescence Resonance Energy Transfer)-based assays, AlphaScreen™ (Amplified Luminescent Proximity Homogeneous Assay), Scintillation Proximity Assay, ELISA (Enzyme-Linked Immunosorbent Assay), SPR (Surface Plasmon Resonance, also known as BIACORE®), isothermal titration calorimetry, differential scanning calorimetry, gel electrophoresis, and chromatography including gel filtration. These and other methods may take advantage of some fusion partner or label of the Fc variant. Assays may employ a variety of detection methods including but not limited to chromogenic, fluorescent, luminescent, or isotopic labels.

The biophysical properties of Fc variants, for example stability and solubility, may be screened using a variety of methods known in the art. Protein stability may be determined by measuring the thermodynamic equilibrium between folded and unfolded states. For example, Fc variants of the present invention may be unfolded using chemical denaturant, heat, or pH, and this transition may be monitored using methods including but not limited to circular dichroism spectroscopy, fluorescence spectroscopy, absorbance spectroscopy, NMR spectroscopy, calorimetry, and proteolysis. As will be appreciated by those skilled in the art, the kinetic parameters of the folding and unfolding transitions may also be monitored using these and other techniques. The solubility and overall structural integrity of an Fc variant may be quantitatively or qualitatively determined using a wide range of methods that are known in the art. Methods which may find use in the present invention for characterizing the biophysical properties of Fc variants include gel electrophoresis, isoelectric focusing, capillary electrophoresis, chromatography such as size exclusion chromatography, ion-exchange chromatography, and reversed-phase high performance liquid chromatography, peptide mapping, oligosaccharide mapping, mass spectrometry, ultraviolet absorbance spectroscopy, fluorescence spectroscopy, circular dichroism spectroscopy, isothermal titration calorimetry, differential scanning calorimetry, analytical ultra-centrifugation, dynamic light scattering, proteolysis, and cross-linking, turbidity measurement, filter retardation assays, immunological assays, fluorescent dye binding assays, protein-staining assays, microscopy, and detection of aggregates via ELISA or other binding assay. Structural analysis employing X-ray crystallographic techniques and NMR spectroscopy may also find use. In one embodiment, stability and/or solubility may be measured by determining the amount of protein solution after some defined period of time. In this assay, the protein may or may not be exposed to some extreme condition, for example elevated temperature, low pH, or the presence of denaturant. Because function typically requires a stable, soluble, and/or well-folded/structured protein, the aforementioned functional and binding assays also provide ways to perform such a measurement. For example, a solution comprising an Fc variant could be assayed for its ability to bind target antigen, then exposed to elevated temperature for one or more defined periods of time, then assayed for antigen binding again. Because unfolded and aggregated protein is not expected to be capable of binding antigen, the amount of activity remaining provides a measure of the Fc variant's stability and solubility.

As is known in the art, a subset of screening methods are those that select for favorable members of a library. The methods are herein referred to as “selection methods”, and these methods find use in for screening IgG variants. When protein libraries are screened using a selection method, only those members of a library that are favorable, that is which meet some selection criteria, are propagated, isolated, and/or observed. As will be appreciated, because only the most fit variants are observed, such methods enable the screening of libraries that are larger than those screenable by methods that assay the fitness of library members individually. Selection is enabled by any method, technique, or fusion partner that links, covalently or noncovalently, the phenotype of a protein with its genotype, that is the function of a protein with the nucleic acid that encodes it. For example the use of phage display as a selection method is enabled by the fusion of library members to the gene III protein. In this way, selection or isolation of IgG variants that meet some criteria, for example binding affinity to the protein's target, also selects for or isolates the nucleic acid that encodes it. Once isolated, the gene or genes encoding Fc variants may then be amplified. This process of isolation and amplification, referred to as panning, may be repeated, allowing favorable IgG variants in the library to be enriched. Nucleic acid sequencing of the attached nucleic acid ultimately allows for gene identification.

A variety of selection methods are known in the art that may find use in for screening protein libraries. These include but are not limited to phage display (Phage display of peptides and proteins: a laboratory manual, Kay et al., 1996, Academic Press, San Diego, Calif., 1996; Lowman et al., 1991, Biochemistry 30:10832-10838; Smith, 1985, Science 228:1315-1317) and its derivatives such as selective phage infection (Malmborg et al., 1997, J Mol Biol 273:544-551), selectively infective phage (Krebber et al., 1997, J Mol Biol 268:619-630), and delayed infectivity panning (Benhar et al., 2000, J Mol Biol 301:893-904), cell surface display (Witrrup, 2001, Curr Opin Biotechnol, 12:395-399) such as display on bacteria (Georgiou et al., 1997, Nat Biotechnol 15:29-34; Georgiou et al., 1993, Trends Biotechnol 11:6-10; Lee et al., 2000, Nat Biotechnol 18:645-648; Jun et al., 1998, Nat Biotechnol 16:576-80), yeast (Boder & Wittrup, 2000, Methods Enzymol 328:430-44; Boder & Wittrup, 1997, Nat Biotechnol 15:553-557), and mammalian cells (Whitehorn et al., 1995, Bio/technology 13:1215-1219), as well as in vitro display technologies (Amstutz et al., 2001, Curr Opin Biotechnol 12:400-405) such as polysome display (Mattheakis et al., 1994, Proc Natl Acad Sci USA 91:9022-9026), ribosome display (Hanes et al., 1997, Proc Natl Acad Sci USA 94:4937-4942), mRNA display (Roberts & Szostak, 1997, Proc Natl Acad Sci USA 94:12297-12302; Nemoto et al., 1997, FEBS Lett 414:405-408), and ribosome-inactivation display system (Zhou et al., 2002, J Am Chem Soc 124, 538-543).

Other selection methods that may find use in include methods that do not rely on display, such as in vivo methods including but not limited to periplasmic expression and cytometric screening (Chen et al., 2001, Nat Biotechnol 19:537-542), the protein fragment complementation assay (Johnsson & Varshaysky, 1994, Proc Natl Acad Sci USA 91:10340-10344; Pelletier et al., 1998, Proc Natl Acad Sci USA 95:12141-12146), and the yeast two hybrid screen (Fields & Song, 1989, Nature 340:245-246) used in selection mode (Visintin et al., 1999, Proc Natl Acad Sci USA 96:11723-11728). In an alternate embodiment, selection is enabled by a fusion partner that binds to a specific sequence on the expression vector, thus linking covalently or noncovalently the fusion partner and associated Fc variant library member with the nucleic acid that encodes them. For example, U.S. Ser. No. 09/642,574; U.S. Ser. No. 10/080,376; U.S. Ser. No. 09/792,630; U.S. Ser. No. 10/023,208; U.S. Ser. No. 09/792,626; U.S. Ser. No. 10/082,671; U.S. Ser. No. 09/953,351; U.S. Ser. No. 10/097,100; U.S. Ser. No. 60/366,658; PCT WO 00/22906; PCT WO 01/49058; PCT WO 02/04852; PCT WO 02/04853; PCT WO 02/08023; PCT WO 01/28702; and PCT WO 02/07466 describe such a fusion partner and technique that may find use in. In an alternative embodiment, in vivo selection can occur if expression of the protein imparts some growth, reproduction, or survival advantage to the cell.

A subset of selection methods referred to as “directed evolution” methods are those that include the mating or breading of favorable sequences during selection, sometimes with the incorporation of new mutations. As will be appreciated by those skilled in the art, directed evolution methods can facilitate identification of the most favorable sequences in a library, and can increase the diversity of sequences that are screened. A variety of directed evolution methods are known in the art that may find use in for screening IgG variants, including but not limited to DNA shuffling (PCT WO 00/42561 A3; PCT WO 01/70947 A3), exon shuffling (U.S. Pat. No. 6,365,377; Kolkman & Stemmer, 2001, Nat Biotechnol 19:423-428), family shuffling (Crameri et al., 1998, Nature 391:288-291; U.S. Pat. No. 6,376,246), RACHIT™ (Coco et al., 2001, Nat Biotechnol 19:354-359; PCT WO 02/06469), STEP and random priming of in vitro recombination (Zhao et al., 1998, Nat Biotechnol 16:258-261; Shao et al., 1998, Nucleic Acids Res 26:681-683), exonuclease mediated gene assembly (U.S. Pat. No. 6,352,842; U.S. Pat. No. 6,361,974), Gene Site Saturation MutaGenesis™ (U.S. Pat. No. 6,358,709), Gene Reassembly™ (U.S. Pat. No. 6,358,709), SCRATCHY (Lutz et al., 2001, Proc Natl Acad Sci USA 98:11248-11253), DNA fragmentation methods (Kikuchi et al., Gene 236:159-167), single-stranded DNA shuffling (Kikuchi et al., 2000, Gene 243:133-137), and AMEsystem™ directed evolution protein engineering technology (Applied Molecular Evolution) (U.S. Pat. No. 5,824,514; U.S. Pat. No. 5,817,483; U.S. Pat. No. 5,814,476; U.S. Pat. No. 5,763,192; U.S. Pat. No. 5,723,323).

In a preferred embodiment, the library is screened using one or more cell-based or in vitro assays. For such assays, Fc variants, purified or unpurified, are typically added exogenously such that cells are exposed to individual variants or groups of variants belonging to a library. These assays are typically, but not always, based on the biology of the ability of the Fc variant to bind to the target antigen and mediate some biochemical event, for example effector functions like cellular lysis, phagocytosis, ligand/receptor binding inhibition, inhibition of growth and/or proliferation, apoptosis and the like. Such assays often involve monitoring the response of cells to Fc variant, for example cell survival, cell death, cellular phagocytosis, cell lysis, change in cellular morphology, or transcriptional activation such as cellular expression of a natural gene or reporter gene. For example, such assays may measure the ability of Fc variants to elicit ADCC, ADCP, or CDC. For some assays additional cells or components, that is in addition to the target cells, may need to be added, for example serum complement, or effector cells such as peripheral blood monocytes (PBMCs), NK cells, macrophages, and the like. Such additional cells may be from any organism, preferably humans, mice, rat, rabbit, and monkey. Crosslinked or monomeric antibodies may cause apoptosis of certain cell lines expressing the antibody's target antigen, or they may mediate attack on target cells by immune cells which have been added to the assay. Methods for monitoring cell death or viability are known in the art, and include the use of dyes, fluorophores, immunochemical, cytochemical, and radioactive reagents. For example, caspase assays or annexin-flourconjugates may enable apoptosis to be measured, and uptake or release of radioactive substrates (e.g., Chromium-51 release assays) or the metabolic reduction of fluorescent dyes such as alamar blue may enable cell growth, proliferationor activation to be monitored. In a preferred embodiment, the DELFIA® EuTDA-based cytotoxicity assay (Perkin Elmer, MA) is used. Alternatively, dead or damaged target cells may be monitored by measuring the release of one or more natural intracellular proteins, for example lactate dehydrogenase. Transcriptional activation may also serve as a method for assaying function in cell-based assays. In this case, response may be monitored by assaying for natural genes or proteins which may be upregulated or down-regulated, for example, the release of certain interleukins may be measured, or alternatively readout may be via a luciferase or GFP-reporter construct. Cell-based assays may also involve the measure of morphological changes of cells as a response to the presence of an Fc variant. Cell types for such assays may be prokaryotic or eukaryotic, and a variety of cell lines that are known in the art may be employed. Alternatively, cell-based screens are performed using cells that have been transformed or transfected with nucleic acids encoding the Fc variants.

In vitro assays include but are not limited to binding assays, ADCC, CDC, cytotoxicity, proliferation, peroxide/ozone release, chemotaxis of effector cells, inhibition of such assays by reduced effector function antibodies; ranges of activities such as >100× improvement or >100× reduction, blends of receptor activation and the assay outcomes that are expected from such receptor profiles.

In Vivo Experimentation

The biological properties of the Fc variants of the present invention may be characterized in cell, tissue, and whole organism experiments. As is know in the art, drugs are often tested in animals, including but not limited to mice, rats, rabbits, dogs, cats, pigs, and monkeys, in order to measure a drug's efficacy for treatment against a disease or disease model, or to measure a drug's pharmacokinetics, toxicity, and other properties. Said animals may be referred to as disease models. With respect to the Fc variants of the present invention, a particular challenge arises when using animal models to evaluate the potential for in-human efficacy of candidate polypeptides—this is due, at least in part, to the fact that Fc variants that have a specific effect on the affinity for a human Fc receptor may not have a similar affinity effect with the orthologous animal receptor. These problems can be further exacerbated by the inevitable ambiguities associated with correct assignment of true orthologues (Mechetina et al., Immunogenetics, 2002 54:463-468, incorporated entirely by reference), and the fact that some orthologues simply do not exist in the animal (e.g., humans possess an FcγRIIa whereas mice do not). Therapeutics are often tested in mice, including but not limited to nude mice, SCID mice, xenograft mice, and transgenic mice (including knockins and knockouts). For example, an Fc variant of the present invention that is intended as an anti-cancer therapeutic may be tested in a mouse cancer model, for example a xenograft mouse. In this method, a tumor or tumor cell line is grafted onto or injected into a mouse, and subsequently the mouse is treated with the therapeutic to determine the ability of the Fc variant to reduce or inhibit cancer growth and metastasis. An alternative approach is the use of a SCID murine model in which immune-deficient mice are injected with human PBLs, conferring a semi-functional and human immune system—with an appropriate array of human FcRs—to the mice that have subsequently been injected with antibodies or Fc-polypeptides that target injected human tumor cells. In such a model, the Fc-polypeptides that target the desired antigen (such as her2/neu on SkOV3 ovarian cancer cells) interact with human PBLs within the mice to engage tumoricidal effector functions. Such experimentation may provide meaningful data for determination of the potential of said Fc variant to be used as a therapeutic. Any organism, preferably mammals, may be used for testing. For example because of their genetic similarity to humans, monkeys can be suitable therapeutic models, and thus may be used to test the efficacy, toxicity, pharmacokinetics, or other property of the Fc variants of the present invention. Tests of the Fc variants of the present invention in humans are ultimately required for approval as drugs, and thus of course these experiments are contemplated. Thus, the Fc variants of the present invention may be tested in humans to determine their therapeutic efficacy, toxicity, pharmacokinetics, and/or other clinical properties.

The Fc variants of the present invention may confer superior performance on Fc-containing therapeutics in animal models or in humans. The receptor binding profiles of such Fc variants, as described in this specification, may, for example, be selected to increase the potency of cytotoxic drugs or to target specific effector functions or effector cells to improve the selectivity of the drug's action. Further, receptor binding profiles can be selected that may reduce some or all effector functions thereby reducing the side-effects or toxicity of such Fc-containing drug. For example, an Fc variant with reduced binding to FcγRIIIa, FcγRI and FcγRIIa can be selected to eliminate most cell-mediated effector function, or an Fc variant with reduced binding to C1q may be selected to limit complement-mediated effector functions. In some contexts, such effector functions are known to have potential toxic effects, therefore eliminating them may increase the safety of the Fc-bearing drug and such improved safety may be characterized in animal models. In some contexts, such effector functions are known to mediate the desirable therapeutic activity, therefore enhancing them may increase the activity or potency of the Fc-bearing drug and such improved activity or potency may be characterized in animal models.

Optimized Fc variants can be tested in a variety of orthotopic tumor models. These clinically relevant animal models are important in the study of pathophysiology and therapy of aggressive cancers like pancreatic, prostate and breast cancer. Immune deprived mice including, but not limited to athymic nude or SCID mice are frequently used in scoring of local and systemic tumor spread from the site of intraorgan (e.g., pancreas, prostate or mammary gland) injection of human tumor cells or fragments of donor patients.

In preferred embodiments, Fc variants of the present invention may be assessed for efficacy in clinically relevant animal models of various human diseases. In many cases, relevant models include various transgenic animals for specific tumor antigens.

Relevant transgenic models such as those that express human Fc receptors (e.g., CD16 including the gamma chain, FcγR1, RIIa/b, and others) could be used to evaluate and test Fc variant antibodies and Fc-fusions in their efficacy. The evaluation of Fc variants by the introduction of human genes that directly or indirectly mediate effector function in mice or other rodents that may enable physiological studies of efficacy in tumor toxicity or other diseases such as autoimmune disorders and RA. Human Fc receptors such as FcγRIIIa may possess polymorphisms such as that in position 158 V or F which would further enable the introduction of specific and combinations of human polymorphisms into rodents. The various studies involving polymorphism-specific FcRs is not limited to this section, however encompasses all discussions and applications of FcRs in general as specified in throughout this application. Fc variants of the present invention may confer superior activity on Fc-containing drugs in such transgenic models, in particular variants with binding profiles optimized for human FcγRIIIa mediated activity may show superior activity in transgenic CD16 mice. Similar improvements in efficacy in mice transgenic for the other human Fc receptors, e.g., FcγRIIa, FcγRI, etc., may be observed for Fc variants with binding profiles optimized for the respective receptors. Mice transgenic for multiple human receptors would show improved activity for Fc variants with binding profiles optimized for the corresponding multiple receptors.

Because of the difficulties and ambiguities associated with using animal models to characterize the potential efficacy of candidate therapeutic antibodies in a human patient, some variant polypeptides of the present invention may find utility as proxies for assessing potential in-human efficacy. Such proxy molecules would preferably mimic—in the animal system—the FcR and/or complement biology of a corresponding candidate human Fc variant. This mimicry is most likely to be manifested by relative association affinities between specific Fc variants and animal vs. human receptors. For example, if one were using a mouse model to assess the potential in-human efficacy of an Fc variant that has enhanced affinity for human FcγRIIIa, an appropriate proxy variant would have enhanced affinity for mouse FcγRIII-2 (mouse CD16-2). Alternatively if one were using a mouse model to assess the potential in-human efficacy of an Fc variant that has reduced affinity for the inhibitory human FcγRIIb, an appropriate proxy variant would have reduced affinity for mouse FcγRII. It should also be noted that the proxy Fc variants could be created in the context of a human Fc variant, an animal Fc variant, or both.

In a preferred embodiment, the testing of Fc variants may include study of efficacy in primates (e.g., cynomolgus monkey model) to facilitate the evaluation of depletion of specific target cells harboring the target antigen. Additional primate models include but not limited to that of the rhesus monkey and Fc polypetides in therapeutic studies of autoimmune, transplantation and cancer.

Toxicity studies are performed to determine the antibody or Fc-fusion related-effects that cannot be evaluated in standard pharmacology profile or occur only after repeated administration of the agent. Most toxicity tests are performed in two species—a rodent and a non-rodent—to ensure that any unexpected adverse effects are not overlooked before new therapeutic entities are introduced into man. In general, these models may measure a variety of toxicities including genotoxicity, chronic toxicity, immunogenicity, reproductive/developmental toxicity and carcinogenicity. Included within the aforementioned parameters are standard measurement of food consumption, bodyweight, antibody formation, clinical chemistry, and macro- and microscopic examination of standard organs/tissues (e.g., cardiotoxicity). Additional parameters of measurement are injection site trauma and the measurement of neutralizing antibodies, if any. Traditionally, monoclonal antibody therepeutics, naked or conjugated are evaluated for cross-reactivity with normal tissues, immunogenicity/antibody production, conjugate or linker toxicity and “bystander” toxicity of radiolabeled species. Nonetheless, such studies may have to be individualized to address specific concerns and following the guidance set by ICH S6 (Safety studies for biotechnological products also noted above). As such, the general principles are that the products are sufficiently well characterized and for which impurities/contaminants have been removed, that the test material is comparable throughout development, and GLP compliance.

The pharmacokinetics (PK) of the Fc variants of the invention can be studied in a variety of animal systems, with the most relevant being non-human primates such as the cynomolgus, rhesus monkeys. Single or repeated i.v./s.c. administrations over a dose range of 6000-fold (0.05-300 mg/kg) can be evaluated for the half-life (days to weeks) using plasma concentration and clearance as well as volume of distribution at a steady state and level of systemic absorbance can be measured. Examples of such parameters of measurement generally include maximum observed plasma concentration (Cmax), the time to reach Cmax (Tmax), the area under the plasma concentration-time curve from time 0 to infinity [AUC(0-inf] and apparent elimination half-life (T1/2). Additional measured parameters could include compartmental analysis of concentration-time data obtained following i.v. administration and bioavailability. Examples of pharmacological/toxicological studies using cynomolgus have been established for Rittman and Zevalin in which monoclonal antibodies to CD20 are cross-reactive. Biodistribution, dosimetry (for radiolabled antibodies), and PK studies can also be done in rodent models. Such studies would evaluate tolerance at all doses administered, toxicity to local tissues, preferential localization to rodent xenograft animal models, depletion of target cells (e.g., CD20 positive cells).

The Fc variants of the present invention may confer superior pharmacokinetics on Fc-containing therapeutics in animal systems or in humans. For example, increased binding to FcRn may increase the half-life and exposure of the Fc-containing drug. Alternatively, decreased binding to FcRn may decrease the half-life and exposure of the Fc-containing drug in cases where reduced exposure is favorable such as when such drug has side-effects.

It is known in the art that the array of Fc receptors is differentially expressed on various immune cell types, as well as in different tissues. Differential tissue distribution of Fc receptors may ultimately have an impact on the pharmacodynamic (PD) and pharmacokinetic (PK) properties of Fc variants of the present invention. Because Fc variants of the presentation have varying affinities for the array of Fc receptors, further screening of the polypeptides for PD and/or PK properties may be extremely useful for defining the optimal balance of PD, PK, and therapeutic efficacy conferred by each candidate polypeptide.

Pharmacodynamic studies may include, but are not limited to, targeting specific tumor cells or blocking signaling mechanisms, measuring depletion of target antigen expressing cells or signals, etc. The Fc variants of the present invention may target particular effector cell populations and thereby direct Fc-containing drugs to recruit certain activities to improve potency or to increase penetration into a particularly favorable physiological compartment. For example, neutrophil activity and localization can be targeted by an Fc variant that preferentially targets FcγRIIIb. Such pharmacodynamic effects may be demonstrated in animal models or in humans.

Clinical Use

The Fc variants of the present invention may find use in a wide range of products. In one embodiment the Fc variant of the present invention is a therapeutic, a diagnostic, or a research reagent, preferably a therapeutic. Alternatively, the Fc variants of the present invention may be used for agricultural or industrial uses.

Therapeutic Uses of Fc Polypeptides

The Fc variants of the present invention find use in a variety of therapeutic uses. As outlined in Figure Z, Fc variants of the invention find use in the treatment of cancer, including, without limitation, Hodgkin's Lymphoma, B-cell malignancies (Non-Hodgkin's Lymphoma, Chronic Lymphocytic Leukemia, myeloma, solid tumors (colorectal cancer, non-small cell lung cancer, kidney cancer, glioblastoma, squamous cell carcinoma of the head and neck, etc.); inflammation; autoimmune diseases, including, without limitation, lupus, rheumatoid arthritis, plaque psoriasis, Crohn's disease, etc., asthma, and allergy, etc.

A “patient” for the purposes of the present invention includes both humans and other animals, preferably mammals and most preferably humans. Thus, the Fc variants of the present invention have both human therapy and veterinary applications. The term “treatment” or “treating” in the present invention is meant to include therapeutic treatment, as well as prophylactic, or suppressive measures for a disease or disorder. Thus, for example, successful administration of an Fc variant prior to onset of the disease results in treatment of the disease. As another example, successful administration of an optimized Fc variant after clinical manifestation of the disease to combat the symptoms of the disease comprises treatment of the disease. “Treatment” and “treating” also encompasses administration of an optimized Fc variant after the appearance of the disease in order to eradicate the disease. Successful administration of an agent after onset and after clinical symptoms have developed, with possible abatement of clinical symptoms and perhaps amelioration of the disease, comprises treatment of the disease. Those “in need of treatment” include mammals already having the disease or disorder, as well as those prone to having the disease or disorder, including those in which the disease or disorder is to be prevented.

In one embodiment, an Fc variant of the present invention is administered to a patient having a disease involving inappropriate expression of a protein or other molecule. Within the scope of the present invention this is meant to include diseases and disorders characterized by aberrant proteins, due for example to alterations in the amount of a protein present, protein localization, posttranslational modification, conformational state, the presence of a mutant or pathogen protein, etc. Similarly, the disease or disorder may be characterized by alterations molecules including but not limited to polysaccharides and gangliosides. An overabundance may be due to any cause, including but not limited to overexpression at the molecular level, prolonged or accumulated appearance at the site of action, or increased activity of a protein relative to normal. Included within this definition are diseases and disorders characterized by a reduction of a protein. This reduction may be due to any cause, including but not limited to reduced expression at the molecular level, shortened or reduced appearance at the site of action, mutant forms of a protein, or decreased activity of a protein relative to normal. Such an overabundance or reduction of a protein can be measured relative to normal expression, appearance, or activity of a protein, and said measurement may play an important role in the development and/or clinical testing of the Fc variants of the present invention.

By “cancer” and “cancerous” herein refer to or describe the physiological condition in mammals that is typically characterized by unregulated cell growth. Examples of cancer include but are not limited to carcinoma, lymphoma, blastoma, sarcoma (including liposarcoma), neuroendocrine tumors, mesothelioma, schwanoma, meningioma, adenocarcinoma, melanoma, and leukemia or lymphoid malignancies.

More particular examples of such cancers include hematologic malignancies, such as Hodgkin's lymphoma; non-Hodgkin's lymphomas (Burkitt's lymphoma, small lymphocytic lymphoma/chronic lymphocytic leukemia, mycosis fungoides, mantle cell lymphoma, follicular lymphoma, diffuse large B-cell lymphoma, marginal zone lymphoma, hairy cell leukemia and lymphoplasmacytic leukemia), tumors of lymphocyte precursor cells, including B-cell acute lymphoblastic leukemia/lymphoma, and T-cell acute lymphoblastic leukemia/lymphoma, thymoma, tumors of the mature T and NK cells, including peripheral T-cell leukemias, adult T-cell leukemia/T-cell lymphomas and large granular lymphocytic leukemia, Langerhans cell histocytosis, myeloid neoplasias such as acute myelogenous leukemias, including AML with maturation, AML without differentiation, acute promyelocytic leukemia, acute myelomonocytic leukemia, and acute monocytic leukemias, myelodysplastic syndromes, and chronic myeloproliferative disorders, including chronic myelogenous leukemia; tumors of the central nervous system such as glioma, glioblastoma, neuroblastoma, astrocytoma, medulloblastoma, ependymoma, and retinoblastoma; solid tumors of the head and neck (e.g., nasopharyngeal cancer, salivary gland carcinoma, and esophagael cancer), lung (e.g., small-cell lung cancer, non-small cell lung cancer, adenocarcinoma of the lung and squamous carcinoma of the lung), digestive system (e.g., gastric or stomach cancer including gastrointestinal cancer, cancer of the bile duct or biliary tract, colon cancer, rectal cancer, colorectal cancer, and anal carcinoma), reproductive system (e.g., testicular, penile, or prostate cancer, uterine, vaginal, vulval, cervical, ovarian, and endometrial cancer), skin (e.g., melanoma, basal cell carcinoma, squamous cell cancer, actinic keratosis), liver (e.g., liver cancer, hepatic carcinoma, hepatocellular cancer, and hepatoma), bone (e.g., osteoclastoma, and osteolytic bone cancers) additional tissues and organs (e.g., pancreatic cancer, bladder cancer, kidney or renal cancer, thyroid cancer, breast cancer, cancer of the peritoneum, and Kaposi's sarcoma), and tumors of the vascular system (e.g., angiosarcoma and hemagiopericytoma).

By “autoimmune diseases” herein include allogenic islet graft rejection, alopecia areata, ankylosing spondylitis, antiphospholipid syndrome, autoimmune Addison's disease, antineutrophil cytoplasmic autoantibodies (ANCA), autoimmune diseases of the adrenal gland, autoimmune hemolytic anemia, autoimmune hepatitis, autoimmune myocarditis, autoimmune neutropenia, autoimmune oophoritis and orchitis, autoimmune thrombocytopenia, autoimmune urticaria, Behcet's disease, bullous pemphigoid, cardiomyopathy, Castleman's syndrome, celiac spruce-dermatitis, chronic fatigue immune disfunction syndrome, chronic inflammatory demyelinating polyneuropathy, Churg-Strauss syndrome, cicatrical pemphigoid, CREST syndrome, cold agglutinin disease, Crohn's disease, dermatomyositis, discoid lupus, essential mixed cryoglobulinemia, factor VIII deficiency, fibromyalgia-fibromyositis, glomerulonephritis, Grave's disease, Guillain-Barre, Goodpasture's syndrome, graft-versus-host disease (GVHD), Hashimoto's thyroiditis, hemophilia A, idiopathic pulmonary fibrosis, idiopathic thrombocytopenia purpura (ITP), IgA neuropathy, IgM polyneuropathies, immune mediated thrombocytopenia, juvenile arthritis, Kawasaki's disease, lichen plantus, lupus erthematosis, Meniere's disease, mixed connective tissue disease, multiple sclerosis, type 1 diabetes mellitus, myasthenia gravis, pemphigus vulgaris, pernicious anemia, polyarteritis nodosa, polychrondritis, polyglandular syndromes, polymyalgia rheumatica, polymyositis and dermatomyositis, primary agammaglobinulinemia, primary biliary cirrhosis, psoriasis, psoriatic arthritis, Reynauld's phenomenon, Reiter's syndrome, rheumatoid arthritis, sarcoidosis, scleroderma, Sjorgen's syndrome, solid organ transplant rejection, stiff-man syndrome, systemic lupus erythematosus, takayasu arteritis, temporal arteristis/giant cell arteritis, thrombotic thrombocytopenia purpura, ulcerative colitis, uveitis, vasculitides such as dermatitis herpetiformis vasculitis, vitiligo, and Wegner's granulomatosis.

By “inflammatory disorders” herein include acute respiratory distress syndrome (ARDS), acute septic arthritis, adjuvant arthritis (Prakken et al., Springer Semin Immunopathol., 2003 August; 25(1):47-63, incorporated entirely by reference), juvenile idiopathic arthritis (de Kleer et al., Arthritis Rheum. 2003 July; 47(7):2001-10, incorporated entirely by reference), allergic encephalomyelitis, allergic rhinitis, allergic vasculitis, allergy, asthma, atherosclerosis, chronic inflammation due to chronic bacterial or viral infectionis, chronic obstructive pulmonary disease (COPD), coronary artery disease, encephalitis, inflammatory bowel disease, inflammatory osteolysis, inflammation associated with acute and delayed hypersensitivity reactions, inflammation associated with tumors, peripheral nerve injury or demyelinating diseases, sciatica, neurodegenerative conditions, inflammation associated with tissue trauma such as burns and ischemia, inflammation due to meningitis, multiple organ injury syndrome, pulmonary fibrosis, sepsis and septic shock, Stevens-Johnson syndrome, undifferentiated arthropy, and undifferentiated spondyloarthropathy.

By “infectious diseases” herein include diseases caused by pathogens such as viruses, bacteria, fungi, protozoa, and parasites. Infectious diseases may be caused by viruses including adenovirus, cytomegalovirus, dengue, Epstein-Barr, hanta, hepatitis A, hepatitis B, hepatitis C, herpes simplex type I, herpes simplex type II, human immunodeficiency virus, (HIV), human papilloma virus (HPV), influenza, measles, mumps, papova virus, polio, respiratory syncytial virus, rinderpest, rhinovirus, rotavirus, rubella, SARS virus, smallpox, viral meningitis, and the like. Infections diseases may also be caused by bacteria including Bacillus antracis, Borrelia burgdorferi, Campylobacter jejuni, Chlamydia trachomatis, Clostridium botulinum, Clostridium tetani, Diptheria, E. coli, Legionella, Helicobacter pylori, Mycobacterium rickettsia, Mycoplasma nesisseria, Pertussis, Pseudomonas aeruginosa, S. pneumonia, Streptococcus, Staphylococcus, Vibria cholerae, Yersinia pestis, and the like. Infectious diseases may also be caused by fungi such as Aspergillus fumigatus, Blastomyces dermatitidis, Candida albicans, Coccidioides immitis, Cryptococcus neoformans, Histoplasma capsulatum, Penicillium marneffei, and the like. Infectious diseases may also be caused by protozoa and parasites such as chlamydia, kokzidioa, leishmania, malaria, rickettsia, trypanosoma, and the like.

Furthermore, antibodies of the present invention may be used to prevent or treat additional conditions including but not limited to heart conditions such as congestive heart failure (CHF), myocarditis and other conditions of the myocardium; skin conditions such as rosecea, acne, and eczema; bone and tooth conditions such as bone loss, osteoporosis, Paget's disease, Langerhans' cell histiocytosis, periodontal disease, disuse osteopenia, osteomalacia, monostotic fibrous dysplasia, polyostotic fibrous dysplasia, bone metastasis, bone pain management, humoral malignant hypercalcemia, periodontal reconstruction, spinal cord injury, and bone fractures; metabolic conditions such as Gaucher's disease; endocrine conditions such as Cushing's syndrome; and neurological conditions.

A number of the receptors that may interact with the Fc variants of the present invention are polymorphic in the human population. For a given patient or population of patients, the efficacy of the Fc variants of the present invention may be affected by the presence or absence of specific polymorphisms in proteins. For example, FcγRIIIa is polymorphic at position 158, which is commonly either V (high affinity) or F (low affinity). Patients with the V/V homozygous genotype are observed to have a better clinical response to treatment with the anti-CD20 antibody Rituxan® (rituximab), likely because these patients mount a stronger NK response (Dall'Ozzo et. al. (2004) Cancer Res. 64:4664-9, incorporated entirely by reference). Additional polymorphisms include but are not limited to FcγRIIa R131 or H131, and such polymorphisms are known to either increase or decrease Fc binding and subsequent biological activity, depending on the polymorphism. Fc variants of the present invention may bind preferentially to a particular polymorphic form of a receptor, for example FcγRIIIa 158 V, or to bind with equivalent affinity to all of the polymorphisms at a particular position in the receptor, for example both the 158V and 158F polymorphisms of FcγRIIIa. In a preferred embodiment, Fc variants of the present invention may have equivalent binding to polymorphisms may be used in an antibody to eliminate the differential efficacy seen in patients with different polymorphisms. Such a property may give greater consistency in therapeutic response and reduce non-responding patient populations. Such variant Fc with indentical binding to receptor polymorphisms may have increased biological activity, such as ADCC, CDC or circulating half-life, or alternatively decreased activity, via modulation of the binding to the relevant Fc receptors. In a preferred embodiment, Fc variants of the present invention may bind with higher or lower affinity to one of the polymorphisms of a receptor, either accentuating the existing difference in binding or reversing the difference. Such a property may allow creation of therapeutics particularly tailored for efficacy with a patient population possessing such polymorphism. For example, a patient population possessing a polymorphism with a higher affinity for an inhibitory receptor such as FcγRIIb could receive a drug containing an Fc variant with reduced binding to such polymorphic form of the receptor, creating a more efficacious drug.

In a preferred embodiment, patients are screened for one or more polymorphisms in order to predict the efficacy of the Fc variants of the present invention. This information may be used, for example, to select patients to include or exclude from clinical trials or, post-approval, to provide guidance to physicians and patients regarding appropriate dosages and treatment options. For example, in patients that are homozygous or heterozygous for FcγRIIIa 158F antibody drugs such as the anti-CD20 mAb, Rituximab are minimally effective (Carton 2002 Blood 99: 754-758; Weng 2003 J. Clin. Oncol. 21:3940-3947, both incorporated entirely by reference); such patients may show a much better clinical response to the antibodies of the present invention. In one embodiment, patients are selected for inclusion in clinical trials for an antibody of the present invention if their genotype indicates that they are likely to respond significantly better to an antibody of the present invention as compared to one or more currently used antibody therapeutics. In another embodiment, appropriate dosages and treatment regimens are determined using such genotype information. In another embodiment, patients are selected for inclusion in a clinical trial or for receipt of therapy post-approval based on their polymorphism genotype, where such therapy contains an Fc variant engineered to be specifically efficacious for such population, or alternatively where such therapy contains an Fc variant that does not show differential activity to the different forms of the polymorphism.

Included in the present invention are diagnostic tests to identify patients who are likely to show a favorable clinical response to an Fc variant of the present invention, or who are likely to exhibit a significantly better response when treated with an Fc variant of the present invention versus one or more currently used antibody therapeutics. Any of a number of methods for determining FcγR polymorphisms in humans known in the art may be used.

Furthermore, the present invention comprises prognostic tests performed on clinical samples such as blood and tissue samples. Such tests may assay for effector function activity, including but not limited to ADCC, CDC, phagocytosis, and opsonization, or for killing, regardless of mechanism, of cancerous or otherwise pathogenic cells. In a preferred embodiment, ADCC assays, such as those described previously, are used to predict, for a specific patient, the efficacy of a given Fc variant of the present invention. Such information may be used to identify patients for inclusion or exclusion in clinical trials, or to inform decisions regarding appropriate dosages and treatment regemins. Such information may also be used to select a drug that contains a particular Fc variant that shows superior activity in such assay.

### Pharmaceutical Formulations, Administration, and Dosing

The therapeutic compositions used in the practice of the foregoing methods can be formulated into pharmaceutical compositions comprising a carrier suitable for the desired delivery method. Suitable carriers include any material that when combined with the therapeutic composition retains the anti-tumor function of the therapeutic composition and is generally non-reactive with the patient's immune system. Examples include, but are not limited to, any of a number of standard pharmaceutical carriers such as sterile phosphate buffered saline solutions, bacteriostatic water, and the like (see, generally, Remington's Pharmaceutical Sciences 16th Edition, A. Osal., Ed., 1980).

### Antibody Compositions for In Vivo Administration

Formulations of the antibodies used in accordance with the present invention are prepared for storage by mixing an antibody having the desired degree of purity with optional pharmaceutically acceptable carriers, excipients or stabilizers (Remington's Pharmaceutical Sciences 16th edition, Osol, A. Ed. [1980]), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONIC™ or polyethylene glycol (PEG).

The formulation herein may also contain more than one active compound as necessary for the particular indication being treated, preferably those with complementary activities that do not adversely affect each other. For example, it may be desirable to provide antibodies with other specifcities. Alternatively, or in addition, the composition may comprise a cytotoxic agent, cytokine, growth inhibitory agent and/or small molecule antagonist. Such molecules are suitably present in combination in amounts that are effective for the purpose intended.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

The formulations to be used for in vivo administration should be sterile, or nearly so. This is readily accomplished by filtration through sterile filtration membranes.

Sustained-release preparations may be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and .gamma. ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

When encapsulated antibodies remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

### Administrative Modalities

The antibodies and compositions of the present invention are administered to a subject, in accord with known methods, such as intravenous administration as a bolus or by continuous infusion over a period of time, by intramuscular, intranasal, transdermal, intraperitoneal, intracerobrospinal, subcutaneous, intra-articular, intrasynovial, intrathecal, oral, topical, or inhalation routes. Intravenous or subcutaneous administration of the antibody is preferred.

Subcutaneous administration may be preferable in some circumstances because the patient may self-administer the pharmaceutical composition. Many protein therapeutics are not sufficiently potent to allow for formulation of a therapeutically effective dose in the maximum acceptable volume for subcutaneous administration. This problem may be addressed in part by the use of protein formulations comprising arginine-HCl, histidine, and polysorbate (see WO 04091658, incorporated entirely by reference). Antibodies of the present invention may be more amenable to subcutaneous administration due to, for example, increased potency, improved serum half-life, or enhanced solubility.

As is known in the art, protein therapeutics are often delivered by IV infusion or bolus. The antibodies of the present invention may also be delivered using such methods. For example, administration may venious be by intravenous infusion with 0.9% sodium chloride as an infusion vehicle.

Pulmonary delivery may be accomplished using an inhaler or nebulizer and a formulation comprising an aerosolizing agent. For example, AERx® inhalable technology commercially available from Aradigm, or Inhance™ pulmonary delivery system commercially available from Nektar Therapeutics may be used. Antibodies of the present invention may be more amenable to intrapulmonary delivery. FcRn is present in the lung, and may promote transport from the lung to the bloodstream (e.g., Syntonix WO 04004798, Bitonti et al. (2004) Proc. Nat. Acad. Sci. 101:9763-8, both incorporated entirely by reference). Accordingly, antibodes that bind FcRn more effectively in the lung or that are released more efficiently in the bloodstream may have improved bioavailability following intrapulmonary administration. Antibodies of the present invention may also be more amenable to intrapulmonary administration due to, for example, improved solubility or altered isoelectric point.

Furthermore, antibodies of the present invention may be more amenable to oral delivery due to, for example, improved stability at gastric pH and increased resistance to proteolysis. Furthermore, FcRn appears to be expressed in the intestinal epithelia of adults (Dickinson et al. (1999) J. Clin. Invest. 104:903-11, incorporated entirely by reference), so antibodies of the present invention with improved FcRn interaction profiles may show enhanced bioavailability following oral administration. FcRn mediated transport of antibodies may also occur at other mucus membranes such as those in the gastrointestinal, respiratory, and genital tracts (Yoshida et al. (2004) Immunity 20:769-83, incorporated entirely by reference).

In addition, any of a number of delivery systems are known in the art and may be used to administer the antibodies of the present invention. Examples include, but are not limited to, encapsulation in liposomes, microparticles, microspheres (eg. PLA/PGA microspheres), and the like. Alternatively, an implant of a porous, non-porous, or gelatinous material, including membranes or fibers, may be used. Sustained release systems may comprise a polymeric material or matrix such as polyesters, hydrogels, poly(vinylalcohol),polylactides, copolymers of L-glutamic acid and ethyl-L-gutamate, ethylene-vinyl acetate, lactic acid-glycolic acid copolymers such as the Lupron Depot®, and poly-D-(−)-3-hydroxyburyric acid. It is also possible to administer a nucleic acid encoding the antibody of the current invention, for example, by retroviral infection, direct injection, or coating with lipids, cell surface receptors, or other transfection agents. In all cases, controlled release systems may be used to release the antibody at or close to the desired location of action.

### Treatment Modalities

In the methods of the invention, therapy is used to provide a positive therapeutic response with respect to a disease or condition. By “positive therapeutic response” is intended an improvement in the disease or condition, and/or an improvement in the symptoms associated with the disease or condition. For example, a positive therapeutic response would refer to one or more of the following improvements in the disease: (1) a reduction in the number of neoplastic cells; (2) an increase in neoplastic cell death; (3) inhibition of neoplastic cell survival; (5) inhibition (i.e., slowing to some extent, preferably halting) of tumor growth; (6) an increased patient survival rate; and (7) some relief from one or more symptoms associated with the disease or condition.

Positive therapeutic responses in any given disease or condition can be determined by standardized response criteria specific to that disease or condition. Tumor response can be assessed for changes in tumor morphology (i.e., overall tumor burden, tumor size, and the like) using screening techniques such as magnetic resonance imaging (MRI) scan, x-radiographic imaging, computed tomographic (CT) scan, bone scan imaging, endoscopy, and tumor biopsy sampling including bone marrow aspiration (BMA) and counting of tumor cells in the circulation.

In addition to these positive therapeutic responses, the subject undergoing therapy may experience the beneficial effect of an improvement in the symptoms associated with the disease.

Thus, for B cell tumors, the subject may experience a decrease in the so-called B symptoms, i.e., night sweats, fever, weight loss, and/or urticaria. For pre-malignant conditions, therapy with a therapeutic agent of the present invention may block and/or prolong the time before development of a related malignant condition, for example, development of multiple myeloma in subjects suffering from monoclonal gammopathy of undetermined significance (MGUS).

An improvement in the disease may be characterized as a complete response. By “complete response” is intended an absence of clinically detectable disease with normalization of any previously abnormal radiographic studies, bone marrow, and cerebrospinal fluid (CSF) or abnormal monoclonal protein in the case of myeloma.

Such a response may persist for at least 4 to 8 weeks, or sometimes 6 to 8 weeks, following treatment according to the methods of the invention. Alternatively, an improvement in the disease may be categorized as being a partial response. By “partial response” is intended at least about a 50% decrease in all measurable tumor burden (i.e., the number of malignant cells present in the subject, or the measured bulk of tumor masses or the quantity of abnormal monoclonal protein) in the absence of new lesions, which may persist for 4 to 8 weeks, or 6 to 8 weeks.

Treatment according to the present invention includes a “therapeutically effective amount” of the medicaments used. A “therapeutically effective amount” refers to an amount effective, at dosages and for periods of time necessary, to achieve a desired therapeutic result.

A therapeutically effective amount may vary according to factors such as the disease state, age, sex, and weight of the individual, and the ability of the medicaments to elicit a desired response in the individual. A therapeutically effective amount is also one in which any toxic or detrimental effects of the antibody or antibody portion are outweighed by the therapeutically beneficial effects.

A “therapeutically effective amount” for tumor therapy may also be measured by its ability to stabilize the progression of disease. The ability of a compound to inhibit cancer may be evaluated in an animal model system predictive of efficacy in human tumors.

Alternatively, this property of a composition may be evaluated by examining the ability of the compound to inhibit cell growth or to induce apoptosis by in vitro assays known to the skilled practitioner. A therapeutically effective amount of a therapeutic compound may decrease tumor size, or otherwise ameliorate symptoms in a subject. One of ordinary skill in the art would be able to determine such amounts based on such factors as the subject's size, the severity of the subject's symptoms, and the particular composition or route of administration selected.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. Parenteral compositions may be formulated in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

The specification for the dosage unit forms of the present invention are dictated by and directly dependent on (a) the unique characteristics of the active compound and the particular therapeutic effect to be achieved, and (b) the limitations inherent in the art of compounding such an active compound for the treatment of sensitivity in individuals.

The efficient dosages and the dosage regimens for the antibodies used in the present invention depend on the disease or condition to be treated and may be determined by the persons skilled in the art.

An exemplary, non-limiting range for a therapeutically effective amount of an antibody used in the present invention is about 0.1-100 mg/kg, such as about 0.1-50 mg/kg, for example about 0.1-20 mg/kg, such as about 0.1-10 mg/kg, for instance about 0.5, about such as 0.3, about 1, or about 3 mg/kg. In another embodiment, the antibody is administered in a dose of 1 mg/kg or more, such as a dose of from 1 to 20 mg/kg, e.g., a dose of from 5 to 20 mg/kg, e.g., a dose of 8 mg/kg.

A medical professional having ordinary skill in the art may readily determine and prescribe the effective amount of the pharmaceutical composition required. For example, a physician or a veterinarian could start doses of the medicament employed in the pharmaceutical composition at levels lower than that required in order to achieve the desired therapeutic effect and gradually increase the dosage until the desired effect is achieved.

In one embodiment, the antibody is administered by infusion in a weekly dosage of from 10 to 500 mg/kg such as of from 200 to 400 mg/kg Such administration may be repeated, e.g., 1 to 8 times, such as 3 to 5 times. The administration may be performed by continuous infusion over a period of from 2 to 24 hours, such as of from 2 to 12 hours.

In one embodiment, the antibody is administered by slow continuous infusion over a long period, such as more than 24 hours, if required to reduce side effects including toxicity.

In one embodiment the antibody is administered in a weekly dosage of from 250 mg to 2000 mg, such as for example 300 mg, 500 mg, 700 mg, 1000 mg, 1500 mg or 2000 mg, for up to 8 times, such as from 4 to 6 times. The administration may be performed by continuous infusion over a period of from 2 to 24 hours, such as of from 2 to 12 hours. Such regimen may be repeated one or more times as necessary, for example, after 6 months or 12 months. The dosage may be determined or adjusted by measuring the amount of compound of the present invention in the blood upon administration by for instance taking out a biological sample and using anti-idiotypic antibodies which target the antigen binding region of the antibody.

In a further embodiment, the antibody is administered once weekly for 2 to 12 weeks, such as for 3 to 10 weeks, such as for 4 to 8 weeks.

In one embodiment, the antibody is administered by maintenance therapy, such as, e.g., once a week for a period of 6 months or more.

In one embodiment, the antibody is administered by a regimen including one infusion of an antibody followed by an infusion of an antibody conjugated to a radioisotope. The regimen may be repeated, e.g., 7 to 9 days later.

As non-limiting examples, treatment according to the present invention may be provided as a daily dosage of an antibody in an amount of about 0.1-100 mg/kg, such as 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of week 1, 2, 3, 4, 5, 6, 7, 8, 9, 10, 11, 12, 13, 14, 15, 16, 17, 18, 19 or 20 after initiation of treatment, or any combination thereof, using single or divided doses of every 24, 12, 8, 6, 4, or 2 hours, or any combination thereof.

In some embodiments the antibody molecule thereof is used in combination with one or more additional therapeutic agents. The additional therapeutic regimes or agents may be used to improve the efficacy or safety of the antibody. Also, the additional therapeutic regimes or agents may be used to treat the same disease or a comorbidity rather than to alter the action of the antibody. For example, an antibody of the present invention may be administered to the patient along with chemotherapy, radiation therapy, or both chemotherapy and radiation therapy. The antibody of the present invention may be administered in combination with one or more other prophylactic or therapeutic agents, including but not limited to cytotoxic agents, chemotherapeutic agents, cytokines, growth inhibitory agents, anti-hormonal agents, kinase inhibitors, anti-angiogenic agents, cardioprotectants, immunostimulatory agents, immunosuppressive agents, agents that promote proliferation of hematological cells, angiogenesis inhibitors, protein tyrosine kinase (PTK) inhibitors, additional antibodies, FcγRIIb or other Fc receptor inhibitors, or other therapeutic agents.

The terms “in combination with” and “co-administration” are not limited to the administration of said prophylactic or therapeutic agents at exactly the same time. Instead, it is meant that the antibody of the present invention and the other agent or agents are administered in a sequence and within a time interval such that they may act together to provide a benefit that is increased versus treatment with only either the antibody of the present invention or the other agent or agents. It is preferred that the antibody and the other agent or agents act additively, and especially preferred that they act synergistically. Such molecules are suitably present in combination in amounts that are effective for the purpose intended. The skilled medical practitioner can determine empirically, or by considering the pharmacokinetics and modes of action of the agents, the appropriate dose or doses of each therapeutic agent, as well as the appropriate timings and methods of administration.

Non-limiting examples of DNA damaging chemotherapeutic agents that are administered in combination with variants of the present invention include topoisomerase I inhibitors (e.g., irinotecan, topotecan, camptothecin and analogs or metabolites thereof, and doxorubicin); topoisomerase II inhibitors (e.g., etoposide, teniposide, and daunorubicin); alkylating agents (e.g., melphalan, chlorambucil, busulfan, thiotepa, ifosfamide, carmustine, lomustine, semustine, streptozocin, decarbazine, methotrexate, mitomycin C, and cyclophosphamide); DNA intercalators (e.g., cisplatin, oxaliplatin, and carboplatin); DNA intercalators and free radical generators such as bleomycin; and nucleoside mimetics (e.g., 5-fluorouracil, capecitibine, gemcitabine, fludarabine, cytarabine, mercaptopurine, thioguanine, pentostatin, and hydroxyurea).

Chemotherapeutic agents that disrupt cell replication include: paclitaxel, docetaxel, and related analogs; vincristine, vinblastin, and related analogs; thalidomide, lenalidomide, and related analogs (e.g., CC-5013 and CC-4047); protein tyrosine kinase inhibitors (e.g., imatinib mesylate and gefitinib); proteasome inhibitors (e.g., bortezomib); NF-κB inhibitors, including inhibitors of IκB kinase; antibodies which bind to proteins overexpressed in cancers and thereby downregulate cell replication (e.g., trastuzumab, rituximab, cetuximab, and bevacizumab); and other inhibitors of proteins or enzymes known to be upregulated, over-expressed or activated in cancers, the inhibition of which downregulates cell replication.

In some embodiments, the antibodies of the invention can be used prior to, concurrent with, or after treatment with Velcade® (bortezomib).

A chemotherapeutic or other cytotoxic agent may be administered as a prodrug. By “prodrug” as used herein is meant a precursor or derivative form of a pharmaceutically active substance that is less cytotoxic to tumor cells compared to the parent drug and is capable of being enzymatically activated or converted into the more active parent form. See, for example Wilman, 1986, Biochemical Society Transactions, 615th Meeting Belfast, 14:375-382; Stella et al., “Prodrugs: A Chemical Approach to Targeted Drug Delivery,” Directed Drug Delivery; and Borchardt et al., (ed.): 247-267, Humana Press, 1985, all incorporated entirely by reference. The prodrugs that may find use with the present invention include but are not limited to phosphate-containing prodrugs, thiophosphate-containing prodrugs, sulfate-containing prodrugs, peptide-containing prodrugs, D-amino acid-modified prodrugs, glycosylated prodrugs, beta-lactam-containing prodrugs, optionally substituted phenoxyacetamide-containing prodrugs or optionally substituted phenylacetamide-containing prodrugs, 5-fluorocytosine and other 5-fluorouridine prodrugs which can be converted into the more active cytotoxic free drug. Examples of cytotoxic drugs that can be derivatized into a prodrug form for use with the antibodies of the present invention include but are not limited to any of the aforementioned chemotherapeutic agents.

In one embodiment, the antibodies of the present invention are administered with one or more additional molecules comprising antibodies or Fc. The antibodies of the present invention may be co-administered with one or more other antibodies that have efficacy in treating the same disease or an additional comorbidity; for example, two antibodies may be administered that recognize two antigens that are overexpressed in a given type of cancer, or two antigens that mediate pathogenesis of an autoimmune or infectious disease.

Examples of anti-cancer antibodies that may be co-administered include, but are not limited to, anti-17-1A cell surface antigen antibodies such as Panorex™ (edrecolomab); anti-4-1BB antibodies; anti-4Dc antibodies; anti-A33 antibodies such as A33 and CDP-833; anti-α4β1 integrin antibodies such as natalizumab; anti-α4β7 integrin antibodies such as LDP-02; anti-αVβ1 integrin antibodies such as F-200, M-200, and SJ-749; anti-αVβ3 integrin antibodies such as abciximab, CNTO-95, Mab-17E6, and Vitaxin™; anti-complement factor 5 (C5) antibodies such as 5G1.1; anti-CA125 antibodies such as OvaRex® (oregovomab); anti-CD3 antibodies such as Nuvion® (visilizumab) and Rexomab; anti-CD4 antibodies such as IDEC-151, MDX-CD4, OKT4A; anti-CD6 antibodies such as Oncolysin B and Oncolysin CD6; anti-CD7 antibodies such as HB2; anti-CD19 antibodies such as B43, MT-103, and Oncolysin B; anti-CD20 antibodies such as 2H7, 2H7.v16, 2H7.v114, 2H7.v115, Bexxar® (tositumomab, I-131 labeled anti-CD20), Rituxan® (rituximab), and Zevalin® (Ibritumomab tiuxetan, Y-90 labeled anti-CD20); anti-CD22 antibodies such as Lymphocide™ (epratuzumab, Y-90 labeled anti-CD22); anti-CD23 antibodies such as IDEC-152; anti-CD25 antibodies such as basiliximab and Zenapax® (daclizumab); anti-CD30 antibodies such as AC10, MDX-060, and SGN-30; anti-CD33 antibodies such as Mylotarg® (gemtuzumab ozogamicin), Oncolysin M, and Smart M195; anti-CD38 antibodies; anti-CD40 antibodies such as SGN-40 and toralizumab; anti-CD40L antibodies such as 5c8, Antova™, and IDEC-131; anti-CD44 antibodies such as bivatuzumab; anti-CD46 antibodies; anti-CD52 antibodies such as Campath® (alemtuzumab); anti-CD55 antibodies such as SC-1; anti-CD56 antibodies such as huN901-DM1; anti-CD64 antibodies such as MDX-33; anti-CD66e antibodies such as XR-303; anti-CD74 antibodies such as IMMU-110; anti-CD80 antibodies such as galiximab and IDEC-114; anti-CD89 antibodies such as MDX-214; anti-CD123 antibodies; anti-CD138 antibodies such as B-B4-DM1; anti-CD146 antibodies such as AA-98; anti-CD148 antibodies; anti-CEA antibodies such as cT84.66, labetuzumab, and Pentacea™; anti-CTLA-4 antibodies such as MDX-101; anti-CXCR4 antibodies; anti-EGFR antibodies such as ABX-EGF, Erbitux® (cetuximab), IMC-C225, and Merck Mab 425; anti-EpCAM antibodies such as Crucell's anti-EpCAM, ING-1, and IS-IL-2; anti-ephrin B2/EphB4 antibodies; anti-Her2 antibodies such as Herceptin®, MDX-210; anti-FAP (fibroblast activation protein) antibodies such as sibrotuzumab; anti-ferritin antibodies such as NXT-211; anti-FGF-1 antibodies; anti-FGF-3 antibodies; anti-FGF-8 antibodies; anti-FGFR antibodies, anti-fibrin antibodies; anti-G250 antibodies such as WX-G250 and Rencarex®; anti-GD2 ganglioside antibodies such as EMD-273063 and TriGem; anti-GD3 ganglioside antibodies such as BEC2, KW-2871, and mitumomab; anti-gpIIb/IIIa antibodies such as ReoPro; anti-heparinase antibodies; anti-Her2/ErbB2 antibodies such as Herceptin® (trastuzumab), MDX-210, and pertuzumab; anti-HLA antibodies such as Oncolym®, Smart 1D10; anti-HM1.24 antibodies; anti-ICAM antibodies such as ICM3; anti-IgA receptor antibodies; anti-IGF-1 antibodies such as CP-751871 and EM-164; anti-IGF-1R antibodies such as IMC-A12; anti-IL-6 antibodies such as CNTO-328 and elsilimomab; anti-IL-15 antibodies such as HuMax™-IL15; anti-KDR antibodies; anti-laminin 5 antibodies; anti-Lewis Y antigen antibodies such as Hu3S193 and IGN-311; anti-MCAM antibodies; anti-Muc1 antibodies such as BravaRex and TriAb; anti-NCAM antibodies such as ERIC-1 and ICRT; anti-PEM antigen antibodies such as Theragyn and Therex; anti-PSA antibodies; anti-PSCA antibodies such as IG8; anti-Ptk antbodies; anti-PTN antibodies; anti-RANKL antibodies such as AMG-162; anti-RLIP76 antibodies; anti-SK-1 antigen antibodies such as Monopharm C; anti-STEAP antibodies; anti-TAG72 antibodies such as CC49-SCA and MDX-220; anti-TGF-r3 antibodies such as CAT-152; anti-TNF-α antibodies such as CDP571, CDP870, D2E7, Humira® (adalimumab), and Remicade® (infliximab); anti-TRAIL-R1 and TRAIL-R2 antibodies; anti-VE-cadherin-2 antibodies; and anti-VLA-4 antibodies such as Antegren™. Furthermore, anti-idiotype antibodies including but not limited to the GD3 epitope antibody BEC2 and the gp72 epitope antibody 105AD7, may be used. In addition, bispecific antibodies including but not limited to the anti-CD3/CD20 antibody Bi20 may be used.

Examples of antibodies that may be co-administered to treat autoimmune or inflammatory disease, transplant rejection, GVHD, and the like include, but are not limited to, anti-α4β7 integrin antibodies such as LDP-02, anti-beta2 integrin antibodies such as LDP-01, anti-complement (C5) antibodies such as 5G1.1, anti-CD2 antibodies such as BTI-322, MEDI-507, anti-CD3 antibodies such as OKT3, SMART anti-CD3, anti-CD4 antibodies such as IDEC-151, MDX-CD4, OKT4A, anti-CD11 a antibodies, anti-CD14 antibodies such as IC14, anti-CD18 antibodies, anti-CD23 antibodies such as IDEC 152, anti-CD25 antibodies such as Zenapax, anti-CD40L antibodies such as 5c8, Antova, IDEC-131, anti-CD64 antibodies such as MDX-33, anti-CD80 antibodies such as IDEC-114, anti-CD147 antibodies such as ABX-CBL, anti-E-selectin antibodies such as CDP850, anti-gpIIb/IIIa antibodies such as ReoPro/Abcixima, anti-ICAM-3 antibodies such as ICM3, anti-ICE antibodies such as VX-740, anti-FcγR1 antibodies such as MDX-33, anti-IgE antibodies such as rhuMab-E25, anti-IL-4 antibodies such as SB-240683, anti-IL-5 antibodies such as SB-240563, SCH55700, anti-IL-8 antibodies such as ABX-IL8, anti-interferon gamma antibodies, and anti-TNFa antibodies such as CDP571, CDP870, D2E7, Infliximab, MAK-195F, anti-VLA-4 antibodies such as Antegren. Examples of other Fc-containing molecules that may be co-administered to treat autoimmune or inflammatory disease, transplant rejection, GVHD, and the like include, but are not limited to, the p75 TNF receptor/Fc fusion Enbrel® (etanercept) and Regeneron's IL-1 trap.

Examples of antibodies that may be co-administered to treat infectious diseases include, but are not limited to, anti-anthrax antibodies such as ABthrax, anti-CMV antibodies such as CytoGam and sevirumab, anti-cryptosporidium antibodies such as CryptoGAM, Sporidin-G, anti-helicobacter antibodies such as Pyloran, anti-hepatitis B antibodies such as HepeX-B, Nabi-HB, anti-HIV antibodies such as HRG-214, anti-RSV antibodies such as felvizumab, HNK-20, palivizumab, RespiGam, and anti-staphylococcus antibodies such as Aurexis, Aurograb, BSYX-A110, and SE-Mab.

Alternatively, the antibodies of the present invention may be co-administered or with one or more other molecules that compete for binding to one or more Fc receptors. For example, co-administering inhibitors of the inhibitory receptor FcγRIIb may result in increased effector function. Similarly, co-administering inhibitors of the activating receptors such as FcγRIIIa may minimize unwanted effector function. Fc receptor inhibitors include, but are not limited to, Fc molecules that are engineered to act as competitive inhibitors for binding to FcγRIIb FcγRIIIa, or other Fc receptors, as well as other immunoglobulins and specifically the treatment called IVIg (intravenous immunoglobulin). In one embodiment, the inhibitor is administered and allowed to act before the antibody is administered. An alternative way of achieving the effect of sequential dosing would be to provide an immediate release dosage form of the Fc receptor inhibitor and then a sustained release formulation of the antibody of the invention. The immediate release and controlled release formulations could be administered separately or be combined into one unit dosage form. Administration of an FcγRIIb inhibitor may also be used to limit unwanted immune responses, for example, anti-Factor VIII antibody response following Factor VIII administration to hemophiliacs.

A variety of other therapeutic agents may find use for administration with the antibodies of the present invention. In one embodiment, the antibody is administered with an anti-angiogenic agent. By “anti-angiogenic agent” as used herein is meant a compound that blocks, or interferes to some degree, the development of blood vessels. The anti-angiogenic factor may, for instance, be a small molecule or a protein, for example, an antibody, Fc fusion, or cytokine, that binds to a growth factor or growth factor receptor involved in promoting angiogenesis. The preferred anti-angiogenic factor herein is an antibody that binds to Vascular Endothelial Growth Factor (VEGF). Other agents that inhibit signaling through VEGF may also be used, for example RNA-based therapeutics that reduce levels of VEGF or VEGF-R expression, VEGF-toxin fusions, Regeneron's VEGF-trap, and antibodies that bind VEGF-R. In an alternate embodiment, the antibody is administered with a therapeutic agent that induces or enhances adaptive immune response, for example, an antibody that targets CTLA-4. Additional anti-angiogenesis agents include, but are not limited to, angiostatin (plasminogen fragment), antithrombin III, angiozyme, ABT-627, Bay 12-9566, benefin, bevacizumab, bisphosphonates, BMS-275291, cartilage-derived inhibitor (CDI), CAI, CD59 complement fragment, CEP-7055, Col 3, combretastatin A-4, endostatin (collagen XVIII fragment), farnesyl transferase inhibitors, fibronectin fragment, gro-beta, halofuginone, heparinases, heparin hexasaccharide fragment, HMV833, human chorionic gonadotropin (hCG), IM-862, interferon alpha, interferon beta, interferon gamma, interferon inducible protein 10 (IP-10), interleukin-12, kringle 5 (plasminogen fragment), marimastat, metalloproteinase inhibitors (eg., TIMPs), 2-methodyestradiol, MMI 270 (CGS 27023A), plasminogen activiator inhibitor (PAD, platelet factor-4 (PF4), prinomastat, prolactin 16 kDa fragment, proliferin-related protein (PRP), PTK 787/ZK 222594, retinoids, solimastat, squalamine, SS3304, SU5416, SU6668, SU11248, tetrahydrocortisol-S, tetrathiomolybdate, thalidomide, thrombospondin-1 (TSP-1), TNP-470, transforming growth factor beta (TGF-β), vasculostatin, vasostatin (calreticulin fragment), ZS6126, and ZD6474.

In a preferred embodiment, the antibody is administered with a tyrosine kinase inhibitor. By “tyrosine kinase inhibitor” as used herein is meant a molecule that inhibits to some extent tyrosine kinase activity of a tyrosine kinase. Examples of such inhibitors include but are not limited to quinazolines, such as PD 153035, 4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo(2,3-d) pyrimidines; curcumin (diferuloyl methane, 4,5-bis (4-fluoroanilino)phthalimide); tyrphostines containing nitrothiophene moieties; PD-0183805 (Warner-Lambert); antisense molecules (e.g., those that bind to ErbB-encoding nucleic acid); quinoxalines (U.S. Pat. No. 5,804,396); tryphostins (U.S. Pat. No. 5,804,396); ZD6474 (Astra Zeneca); PTK-787 (Novartis/Schering A G); pan-ErbB inhibitors such as C1-1033 (Pfizer); Affinitac (ISIS 3521; Isis/Lilly); Imatinib mesylate (STI571,Gleevec®; Novartis); PM 166 (Novartis); GW2016 (Glaxo SmithKline); C1-1033 (Pfizer); EKB-569 (Wyeth); Semaxinib (Sugen); ZD6474 (AstraZeneca); PTK-787 (Novartis/Schering AG); INC-1C11 (Imclone); or as described in any of the following patent publications: U.S. Pat. No. 5,804,396; PCT WO 99/09016 (American Cyanimid); PCT WO 98/43960 (American Cyanamid); PCT WO 97/38983 (Warner-Lambert); PCT WO 99/06378 (Warner-Lambert); PCT WO 99/06396 (Warner-Lambert); PCT WO 96/30347 (Pfizer, Inc); PCT WO 96/33978 (AstraZeneca); PCT WO96/3397 (AstraZeneca); PCT WO 96/33980 (AstraZeneca), gefitinib (IRESSA™, ZD1839, AstraZeneca), and OSI-774 (Tarceva™, OSI Pharmaceuticals/Genentech), all patent publications incorporated entirely by reference.

In another embodiment, the antibody is administered with one or more immunomodulatory agents. Such agents may increase or decrease production of one or more cytokines, up- or down-regulate self-antigen presentation, mask MHC antigens, or promote the proliferation, differentiation, migration, or activation state of one or more types of immune cells. Immunomodulatory agents include but not limited to: non-steroidal anti-inflammatory drugs (NSAIDs) such as asprin, ibuprofed, celecoxib, diclofenac, etodolac, fenoprofen, indomethacin, ketoralac, oxaprozin, nabumentone, sulindac, tolmentin, rofecoxib, naproxen, ketoprofen, and nabumetone; steroids (e.g., glucocorticoids, dexamethasone, cortisone, hydroxycortisone, methylprednisolone, prednisone, prednisolone, trimcinolone, azulfidineicosanoids such as prostaglandins, thromboxanes, and leukotrienes; as well as topical steroids such as anthralin, calcipotriene, clobetasol, and tazarotene); cytokines such as TGFb, IFNa, IFNb, IFNg, IL-2, IL-4, IL-10; cytokine, chemokine, or receptor antagonists including antibodies, soluble receptors, and receptor-Fc fusions against BAFF, B7, CCR2, CCR5, CD2, CD3, CD4, CD6, CD7, CD8, CD11, CD14, CD15, CD17, CD18, CD20, CD23, CD28, CD40, CD40L, CD44, CD45, CD52, CD64, CD80, CD86, CD147, CD152, complement factors (C5, D) CTLA4, eotaxin, Fas, ICAM, ICOS, IFNa, IFNI3, IFN1, IFNAR, IgE, IL-1, IL-2, IL-2R, IL-4, IL-5R, IL-6, IL-8, IL-9 IL-12, IL-13, IL-13R1, IL-15, IL-18R, IL-23, integrins, LFA-1, LFA-3, MHC, selectins, TGFI3, TNFα, TNFI3, TNF-R1, T-cell receptor, including Enbrel® (etanercept), Humira® (adalimumab), and Remicade® (infliximab); heterologous anti-lymphocyte globulin; other immunomodulatory molecules such as 2-amino-6-aryl-5 substituted pyrimidines, anti-idiotypic antibodies for MHC binding peptides and MHC fragments, azathioprine, brequinar, bromocryptine, cyclophosphamide, cyclosporine A, D-penicillamine, deoxyspergualin, FK506, glutaraldehyde, gold, hydroxychloroquine, leflunomide, malononitriloamides (e.g., leflunomide), methotrexate, minocycline, mizoribine, mycophenolate mofetil, rapamycin, and sulfasasazine.

In an alternate embodiment, antibody of the present invention are administered with a cytokine. By “cytokine” as used herein is meant a generic term for proteins released by one cell population that act on another cell as intercellular mediators. Examples of such cytokines are lymphokines, monokines, and traditional polypeptide hormones. Included among the cytokines are growth hormone such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-alpha and -beta; mullerian-inhibiting substance; mouse gonadotropin-associated peptide; inhibin; activin; vascular endothelial growth factor; integrin; thrombopoietin (TPO); nerve growth factors such as NGF-beta; platelet-growth factor; transforming growth factors (TGFs) such as TGF-alpha and TGF-beta; insulin-like growth factor-I and -II; erythropoietin (EPO); osteoinductive factors; interferons such as interferon-alpha, beta, and -gamma; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1alpha, IL-2, IL-3, IL-4, IL-5, IL-6, IL-7, IL-8, IL-9, IL-10, IL-11, IL-12; IL-15, a tumor necrosis factor such as TNF-alpha or TNF-beta; and other polypeptide factors including LIF and kit ligand (KL). As used herein, the term cytokine includes proteins from natural sources or from recombinant cell culture, and biologically active equivalents of the native sequence cytokines.

In a preferred embodiment, cytokines or other agents that stimulate cells of the immune system are co-administered with the antibody of the present invention. Such a mode of treatment may enhance desired effector function. For example, agents that stimulate NK cells, including but not limited to IL-2 may be co-administered. In another embodiment, agents that stimulate macrophages, including but not limited to C5a, formyl peptides such as N-formyl-methionyl-leucyl-phenylalanine (Beigier-Bompadre et al. (2003) Scand. J. Immunol. 57: 221-8, incorporated entirely by reference), may be co-administered. Also, agents that stimulate neutrophils, including but not limited to G-CSF, GM-CSF, and the like may be administered. Furthermore, agents that promote migration of such immunostimulatory cytokines may be used. Also additional agents including but not limited to interferon gamma, IL-3 and IL-7 may promote one or more effector functions.

In an alternate embodiment, cytokines or other agents that inhibit effector cell function are co-administered with the antibody of the present invention. Such a mode of treatment may limit unwanted effector function.

In an additional embodiment, the antibody is administered with one or more antibiotics, including but not limited to: aminoglycoside antibiotics (eg. apramycin, arbekacin, bambermycins, butirosin, dibekacin, gentamicin, kanamycin, neomycin, netilmicin, paromomycin, ribostamycin, sisomycin, spectrinomycin), aminocyclitols (eg. sprctinomycin), amphenicol antibiotics (eg. azidamfenicol, chloramphenicol, florfrnicol, and thiamphemicol), ansamycin antibiotics (eg. rifamide and rifampin), carbapenems (eg. imipenem, meropenem, panipenem); cephalosporins (eg. cefaclor, cefadroxil, cefamandole, cefatrizine, cefazedone, cefozopran, cefpimizole, cefpiramide, cefpirome, cefprozil, cefuroxine, cefixime, cephalexin, cephradine), cephamycins (cefbuperazone, cefoxitin, cefminox, cefmetazole, and cefotetan); lincosamides (eg. clindamycin, lincomycin); macrolide (eg. azithromycin, brefeldin A, clarithromycin, erythromycin, roxithromycin, tobramycin), monobactams (eg. aztreonam, carumonam, and tigemonam); mupirocin; oxacephems (eg. flomoxef, latamoxef, and moxalactam); penicillins (eg. amdinocillin, amdinocillin pivoxil, amoxicillin, bacampicillin, bexzylpenicillinic acid, benzylpenicillin sodium, epicillin, fenbenicillin, floxacillin, penamecillin, penethamate hydriodide, penicillin o-benethamine, penicillin O, penicillin V, penicillin V benzoate, penicillin V hydrabamine, penimepicycline, and phencihicillin potassium); polypeptides (eg. bacitracin, colistin, polymixin B, teicoplanin, vancomycin); quinolones (amifloxacin, cinoxacin, ciprofloxacin, enoxacin, enrofloxacin, feroxacin, flumequine, gatifloxacin, gemifloxacin, grepafloxacin, lomefloxacin, moxifloxacin, nalidixic acid, norfloxacin, ofloxacin, oxolinic acid, pefloxacin, pipemidic acid, rosoxacin, rufloxacin, sparfloxacin, temafloxacin, tosufloxacin, trovafloxacin); rifampin; streptogramins (eg. quinupristin, dalfopristin); sulfonamides (sulfanilamide, sulfamethoxazole); tetracyclenes (chlortetracycline, demeclocycline hydrochloride, demethylchlortetracycline, doxycycline, duramycin, minocycline, neomycin, oxytetracycline, streptomycin, tetracycline, vancomycin).

Anti-fungal agents such as amphotericin B, ciclopirox, clotrimazole, econazole, fluconazole, flucytosine, itraconazole, ketoconazole, niconazole, nystatin, terbinafine, terconazole, and tioconazole may also be used.

Antiviral agents including protease inhibitors, reverse transcriptase inhibitors, and others, including type I interferons, viral fusion inhibitors, and neuramidase inhibitors, may also be used. Examples of antiviral agents include, but are not limited to, acyclovir, adefovir, amantadine, amprenavir, clevadine, enfuvirtide, entecavir, foscarnet, gangcyclovir, idoxuridine, indinavir, lopinavir, pleconaril, ribavirin, rimantadine, ritonavir, saquinavir, trifluridine, vidarabine, and zidovudine, may be used.

The antibodies of the present invention may be combined with other therapeutic regimens. For example, in one embodiment, the patient to be treated with an antibody of the present invention may also receive radiation therapy. Radiation therapy can be administered according to protocols commonly employed in the art and known to the skilled artisan. Such therapy includes but is not limited to cesium, iridium, iodine, or cobalt radiation. The radiation therapy may be whole body irradiation, or may be directed locally to a specific site or tissue in or on the body, such as the lung, bladder, or prostate. Typically, radiation therapy is administered in pulses over a period of time from about 1 to 2 weeks. The radiation therapy may, however, be administered over longer periods of time. For instance, radiation therapy may be administered to patients having head and neck cancer for about 6 to about 7 weeks. Optionally, the radiation therapy may be administered as a single dose or as multiple, sequential doses. The skilled medical practitioner can determine empirically the appropriate dose or doses of radiation therapy useful herein. In accordance with another embodiment of the invention, the antibody of the present invention and one or more other anti-cancer therapies are employed to treat cancer cells ex vivo. It is contemplated that such ex vivo treatment may be useful in bone marrow transplantation and particularly, autologous bone marrow transplantation. For instance, treatment of cells or tissue(s) containing cancer cells with antibody and one or more other anti-cancer therapies, such as described above, can be employed to deplete or substantially deplete the cancer cells prior to transplantation in a recipient patient.

It is of course contemplated that the antibodies of the invention may employ in combination with still other therapeutic techniques such as surgery or phototherapy.

All cited references are herein expressly incorporated by reference in their entirety.

### Examples

Examples are provided below to illustrate the present invention. These examples are not meant to constrain the present invention to any particular application or theory of operation.

Fc variants and Fc variant libraries were designed using computational- and sequence-based methods as described in U.S. Ser. No. 10/672,280 and U.S. Ser. No. 10/822,231. Experimental libraries were designed in successive rounds of computational and experimental screening. Design of subsequent Fc libraries benefitted from feedback from prior libraries, and thus typically comprised combinations of Fc variants that showed favorable properties in the previous screen. FIG. 97 shows residues at which amino acid modifications were made in the Fc variants of the present invention, mapped onto the human Fc/FcγRIIIb structure. The entire set of Fc variants that were constructed and experimentally tested is shown in FIG. 24.

### Example 1. Fc Variants with Reduced FcγR- and Complement-Mediated Effector Function

For some applications it may be favorable to reduce or eliminate binding to one or more FcγRs, or reduce or eliminate one or more FcγR- or complement-mediated effector functions including but not limited to ADCC, ADCP, and/or CDC. This is often the case for therapeutic antibodies whose mechanism of action involves blocking or antagonism but not killing of the cells bearing target antigen. In these cases depletion of target cells is undesirable and can be considered a side effect. Effector function can also be a problem for radiolabeled antibodies, referred to as radioconjugates, and antibodies conjugated to toxins, referred to as immunotoxins. These drugs can be used to destroy cancer cells, but the recruitment of immune cells via Fc interaction with FcγRs brings healthy immune cells in proximity to the deadly payload (radiation or toxin), resulting in depletion of normal lymphoid tissue along with targeted cancer cells.

A previously unconsidered advantage of ablated FcγR- and complement-binding is that in cases where effector function is not needed, binding to FcγR and complement may effectively reduce the active concentration of drug. Binding to Fc ligands may localize an antibody or Fc fusion to cell surfaces or in complex with serum proteins wherein it is less active or inactive relative to when it is free (uncomplexed). This may be due to decreased effective concentration at binding sites where the antibody is desired, or perhaps Fc ligand binding may put the Fc polypeptide in a conformation in which it is less active than it would be if it were unbound. An additional consideration is that FcγR-receptors may be one mechanism of antibody turnover, and can mediate uptake and processing by antigen presenting cells such as dendritic cells and macrophages. This may affect the pharmacokinetics (or in vivo half-life) of the antibody or Fc fusion and its immunogenicity, both of which are critical parameters of clinical performance.

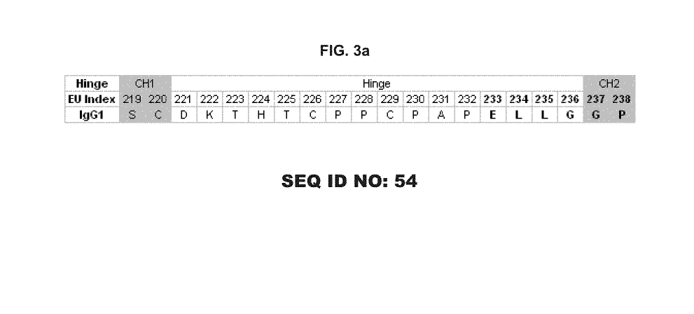

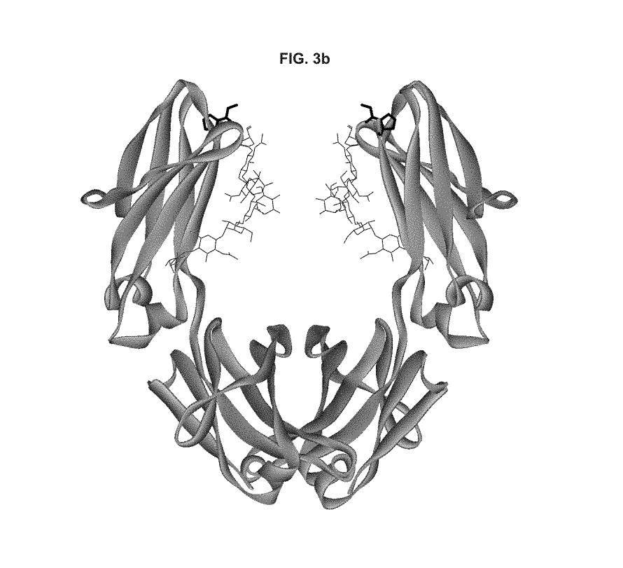

Variants comprising insertions, deletions, and substitutions in the Fc region were engineered to reduce or ablate interaction with FcγRs and complement. Insertions and deletions are not commonly used in protein engineering strategies to modulate binding interactions because of the potential for large perturbations to protein structure and stability. However as illustrated in FIG. 3, the flexible hinge region of an antibody may be uniquely amenable to engineering of insertions and deletions. The hinge region, defined herein from position 221-236, contains part of the Fc region, and contains some binding determinants for interaction with Fc receptors. The FcγR binding site begins approximately at residue 233, yet structurally, the CH2 domain begins at position 237. Thus, it may be that insertions and deletions at and N-terminal to position 237 can be used to modulate interaction with FcγRs and complement, yet without affecting the stability and fidelity of the structured CH2 domain.

FIG. 4. lists a series of variants that were designed to reduce or ablate interaction with FcγRs and complement. The variants were constructed in the context of an antibody comprising the Fv region of the anti-Her2 antibody trastuzumab and the constant heavy chain of the human IgG1. Human IgG2 and IgG4 versions were also constructed to compare effector function of the Fc variants with naturally existing IgG antibodies.

AlphaScreen™ binding data for select Fc variants with substantially reduced or ablated FcγR binding are shown in FIGS. 141a and 141b. These Fc variants, as well as their use in combination, may find use for eliminating effector function when desired, for example in antibodies and Fc fusions whose mechanism of action involves blocking or antagonism but not killing of the cells bearing target antigen.

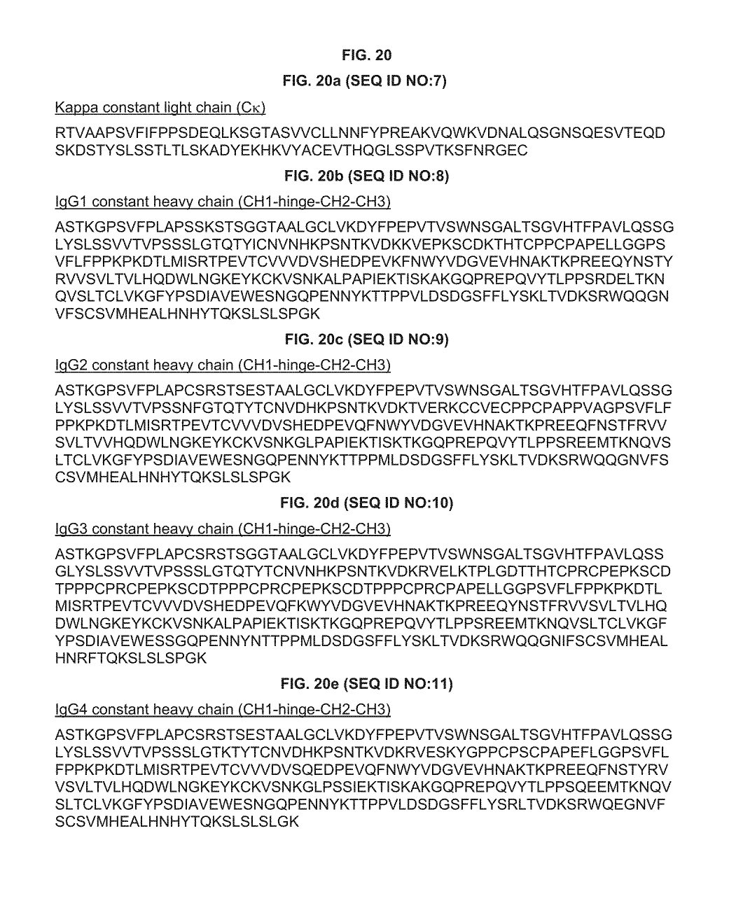

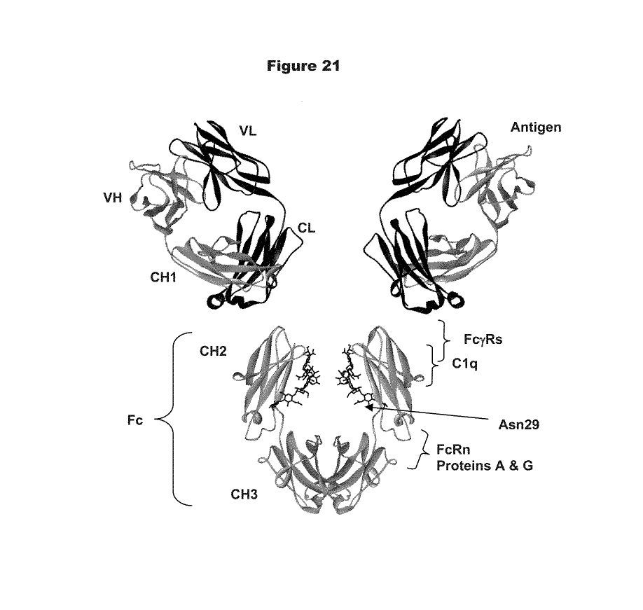

The genes for the variable region of anti-Her2 antibody trastuzumab (Carter et al., 1992, Proc Natl Acad Sci USA 89:4285-4289) were constructed using recursive PCR, and subcloned into the mammalian expression vector pcDNA3.1Zeo (Invitrogen) comprising the full length light kappa (Cκ) constant region for the light chain, or the heavy chain IgG1, IgG2, or IgG4 constant regions for the heavy chain. Amino acid modifications were constructed in the Fc region of the antibodies in the pcDNA3.1Zeo vector using quick-change mutagenesis techniques (Stratagene). DNA was sequenced to confirm the fidelity of the sequences. Plasmids containing heavy chain gene (VH-CH1-CH2-CH3) (wild-type or variants) were co-transfected with plasmid containing light chain gene (VL-Cκ) into 293T cells. Media were harvested 5 days after transfection, and antibodies were purified from the supernatant using protein A affinity chromatography (Pierce). The sequences of the Cκ and IgG isotype constant chains are shown in FIG. 20.

In order to evaluate the interaction of the antibodies with Fc receptors, the extracellular regions of human Fc receptors R131 and H131 FcγRIIa, and V158 and F158 FcγRIIIa containing C-terminal 6×His tags were obtained by PCR from clones obtained from the Mammalian Gene Collection (MGC), or generated de novo using recursive PCR. Receptors were expressed in 293T cells and purified using nickel affinity chromatography. His-tagged extracellular regions of human FcγRI and FcγRIIb were obtained from R&D Systems.

Binding affinity to human FcγRs by Fc variant antibodies was measured using surface plasmon resonance (SPR), also referred to as BIAcore. Surface plasmon resonance measurements were performed using a Biacore 3000 instrument (Biacore). Antibodies were captured onto an immobilized protein A/G (Pierce) CMS biosensor chip (Biacore), generated using a standard primary amine coupling protocol. All measurements were performed in HBS-EP (0.01 M HEPES pH 7.4, 0.15 M NaCl, 3 mM EDTA, 0.005% v/v surfactant P20, Biacore) and glycine buffer (10 mM glycine-HCl, pH 1.5, Biacore) was used for the Protein A/G surface regeneration. All antibodies (50 nM in HBS-EP) were immobilized on the protein A/G surface for 5 minutes at 1 ul/min. Fc receptors in serial dilutions were injected over antibody bound surface for 2 min at 20 ul/min followed by 2 or 3 min dissociation phase. After each cycle the Protein A/G surface was regenerated by injecting glycine buffer (pH 1.5) for 30 s at 10 ul/min. Data were processed by zeroing time and response before the injection of receptor and by subtracting of appropriate non-specific signals (response of reference channel and injection of running buffer). Kinetic analysis was performed by global fitting of binding data with a 1:1 binding model (Langmuir) using the BIAevaluation software.

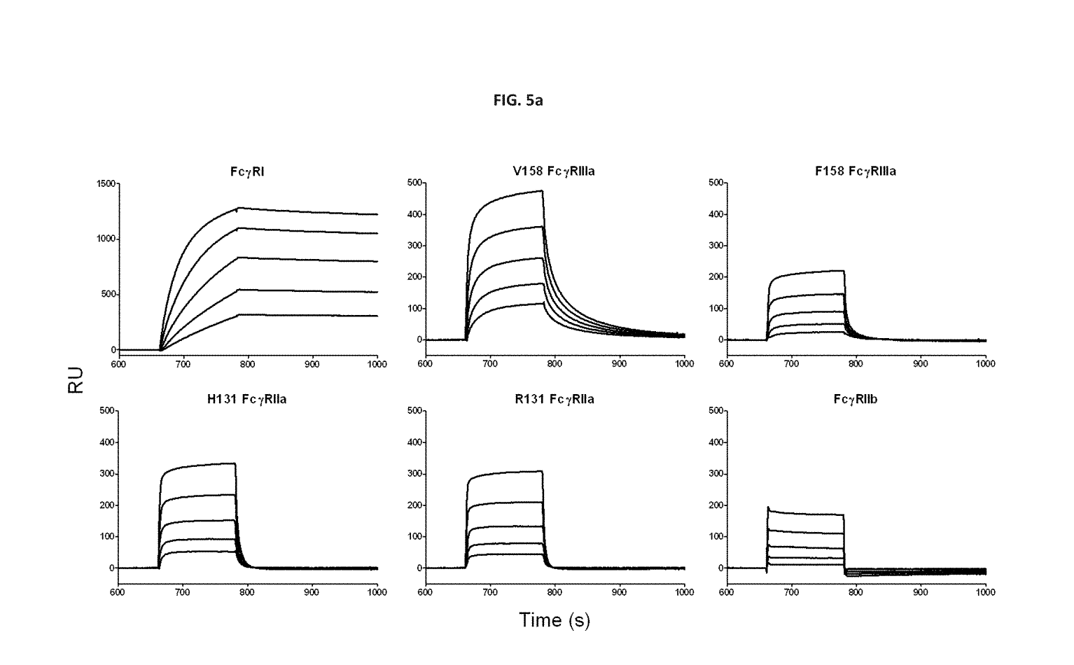

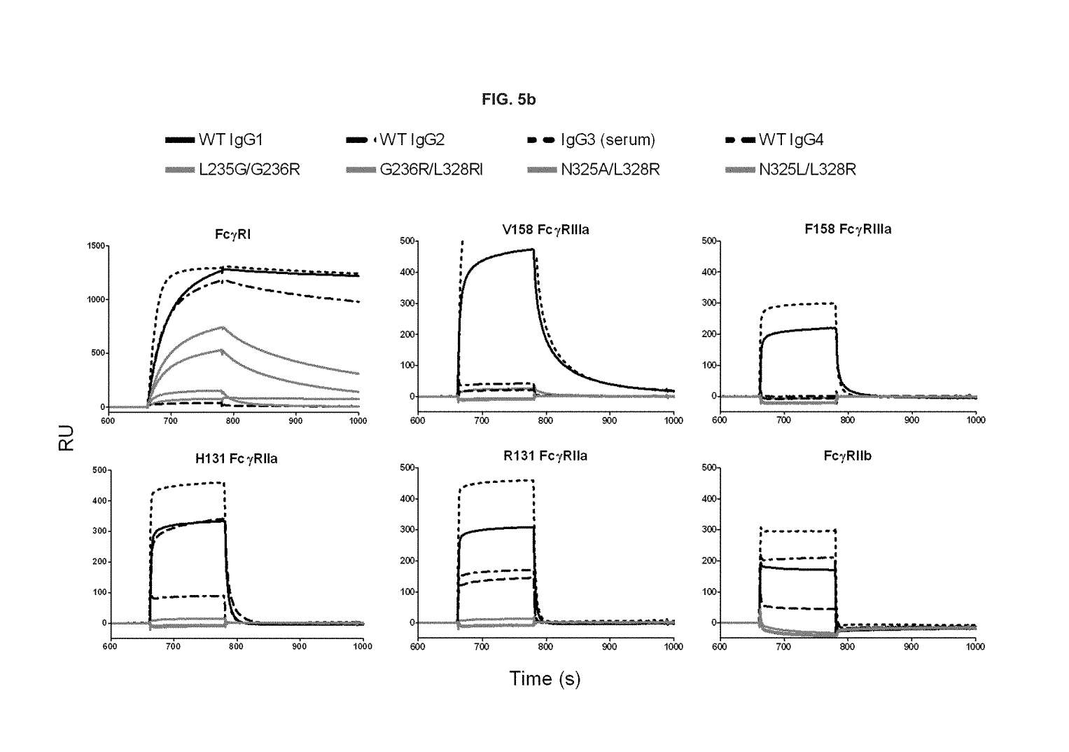

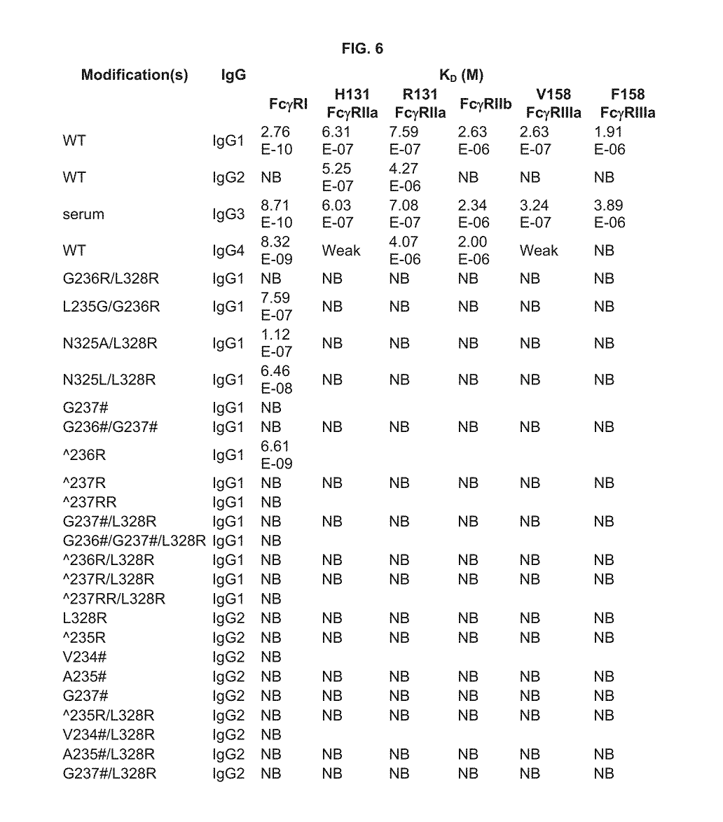

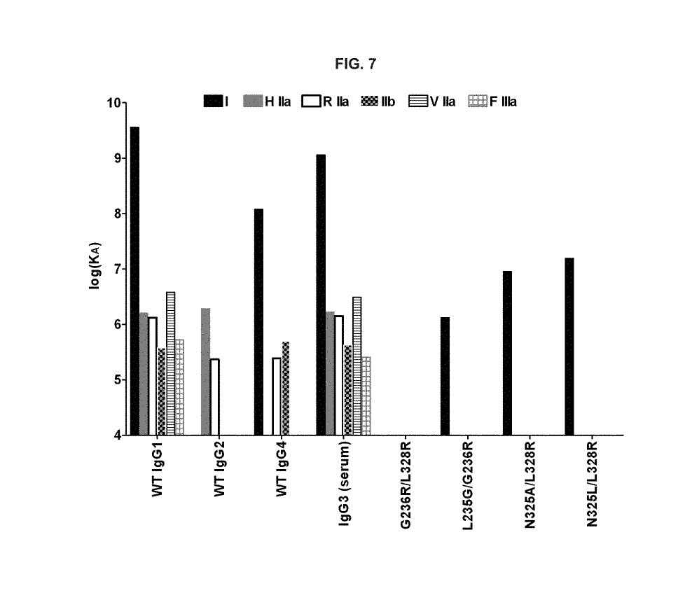

FIG. 5a shows the normalized SPR sensorgrams for each concentration for binding of WT IgG1 to the human Fc receptors FcγRI, both isoforms (H131 and R131) FcγRIIa, FcγRIIb, and both isofoforms (V158 and F158) of FcγRIIIa. An identical experiment was run for the other IgG isotypes (monoclonal IgG1, IgG2, and IgG4 with anti-Her2 variable region and polyclonal serum IgG3 purchased commercially) as well as select variants. FIG. 5b shows representative sensorgrams from each antibody at the highest concentration for each receptor. The higher amplitude and slower off-rates observed with IgG1 and IgG3 relative to IgG2 and IgG4 are consistent with the weaker binding of the latter. In contrast, no binding was observed for all of the variants tested, with the exception of FcγRI for some of the variants. Langmuir fits of the Biacore data for all the variants provided equilibrium K_{D}s (FIG. 6). FIG. 7 shows a plot of the affinities (K_{A}=1/K_{D}) on a logarithmic scale for binding of each antibody to each receptor. As can be seen, variant G236R/L328R shows no binding to any of the FcγRs. Variants L235G/G236R, N325A/L328R, and N325L/L328R show no binding to FcγRII and FcγRIII receptors, and show some albeit reduced binding to FcγR1.

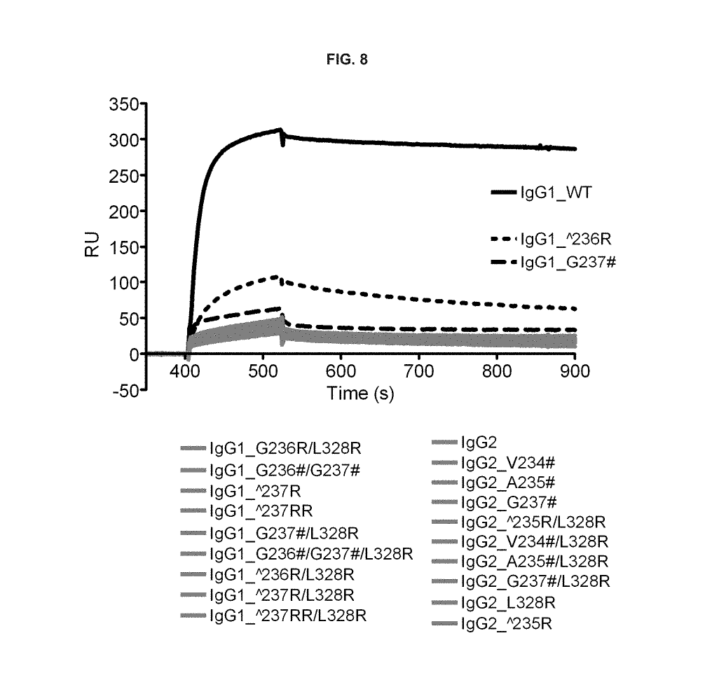

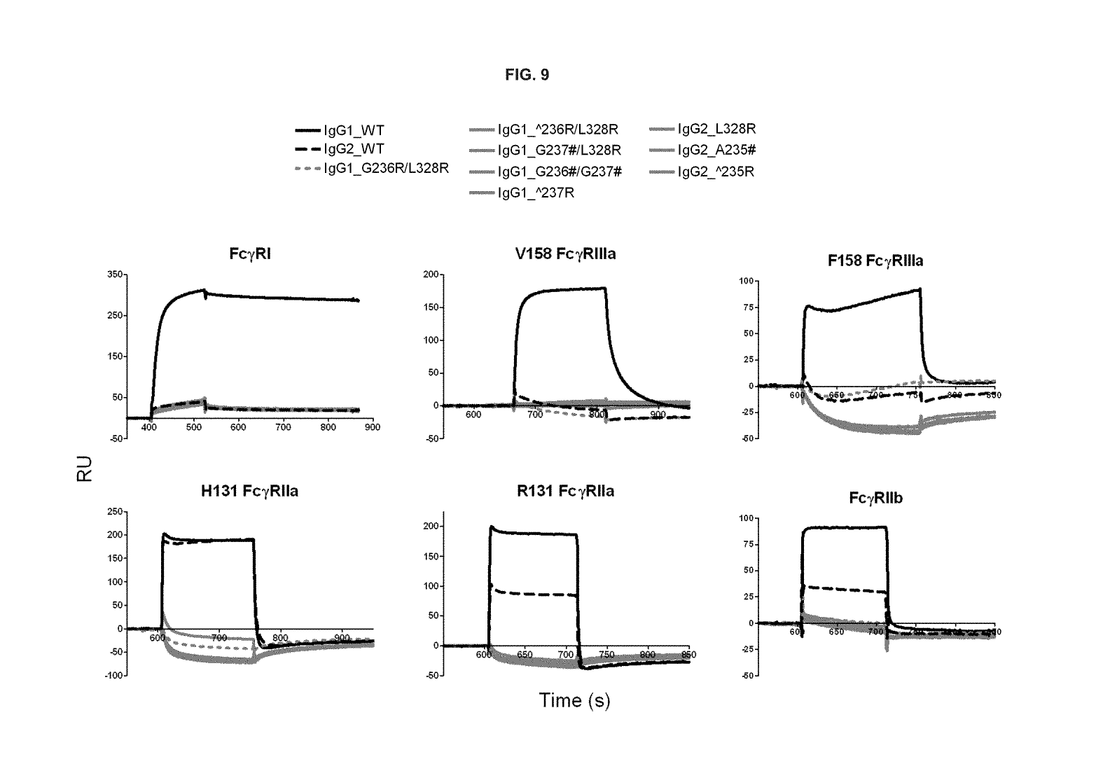

Because binding to FcγRI was the most difficult among the Fc receptors to reduce, this receptor was used as the primary screen for the other variants. Other variants comprising insertions and deletions in the hinge region, as well as in some cases substitutions in the Fc region, were screened for binding to FcγRI using Biacore as described above. FIG. 8 shows the sensorgrams at the highest receptor concentration for all of the variants and WT IgG1. As can be seen, ̂236R, which has an arginine insertion after position 236, and G237#, which has a deletion of G237, have reduced but observable binding to FcγR1. In contrast, all other variants, comprising a variety of insertions and deletions in the hinge, as well as substitutions in the Fc region, have completely ablated binding to the high affinity receptor FcγR1. Select variants were tested for binding to all signaling FcγRs by Biacore. FIG. 9 shows sensorgrams at the highest concentration for binding of these variants to human FcγR1, FcγRIIa, FcγRIIb, and FcγRIIIa. As can be seen, these variants show no detectable binding to any of the human FcγRs. The binding data for all of the variants to all of the receptors tested are summarized in FIG. 6.

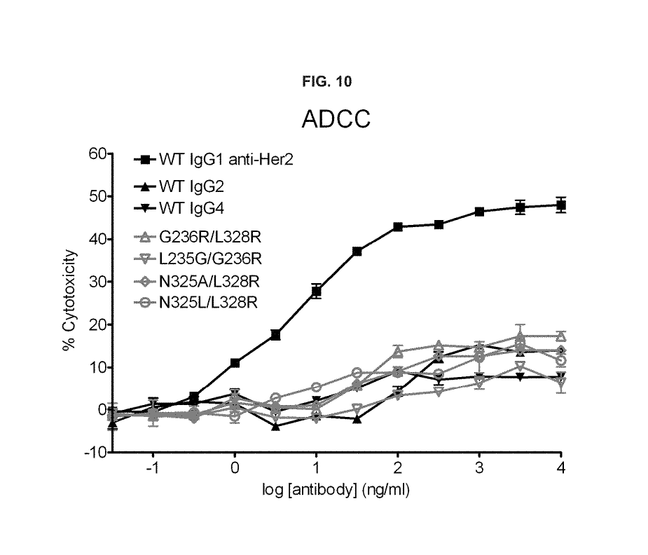

To assess the impact of the variants with reduced/ablated FcγR binding, select variants were tested for their capacity to mediate antibody dependent cellular cytotoxicity (ADCC). Human PBMCs were used as effector cells, and the Her2+ cell line Sk-Br-3 was used as target cells. PBMCs were purified from leukopacks using a Ficoll gradient, and ADCC was measured by LDH release. Target cells were seeded into 96-well plates and opsonized using native IgG or Fc variant antibodies at the indicated concentrations. Triton X100 and PBMCs alone were run as controls. Effector cells were added and plates were incubated at 37° C., 5% CO2 for 4 h. Cells were then incubated with LDH reaction mixture for 10 min, and fluorescence was measured using a Wallac Victor2 fluorometer (PerkinElmer). Fluorescence due to spontaneous PBMC and target cell lysis (without antibodies) was subtracted from experimental values (with antibodies), normalized to maximal (Triton) and minimal (no Triton) lysis, and fit to a sigmoidal dose-response model. FIG. 10 shows that variants with reduced FcγR binding do not mediate ADCC, similarly to WT IgG2 and IgG4 and in contrast to WT IgG1 which binds with high affinity to FcγRs, particularly FcγRIIIa. PBMC ADCC is dominatetd by NK cells, which only express FcγRIIIa.

Monocyte-derived effector cells, including for example macrophages, express not only FcγRIIIa, but also FcγRI, FcγRIIa, and the inhibitory receptor FcγRIIb. Macrophages are phagocytes that act as scavengers to engulf dead cells, foreign substances, and other debris. Importantly, macrophages are professional antigen presenting cells (APCs), taking up pathogens and foreign structures in peripheral tissues, then migrating to secondary lymphoid organs to initiate adaptive immune responses by activating naive T cells. Unlike NK cells, macrophages express the range of FcγRs, and thus their activation and function may be dependent on engagement of antibody immune complexes with receptors other than only FcγRIIIa. To evaluate the effect of ablation of FcγR affinity, the ̂236R/L328R variant was tested for its capacity to mediate macrophage antibody dependent cellular phagocytosis (ADCP). WT IgG1 was also run as a comparator and control.

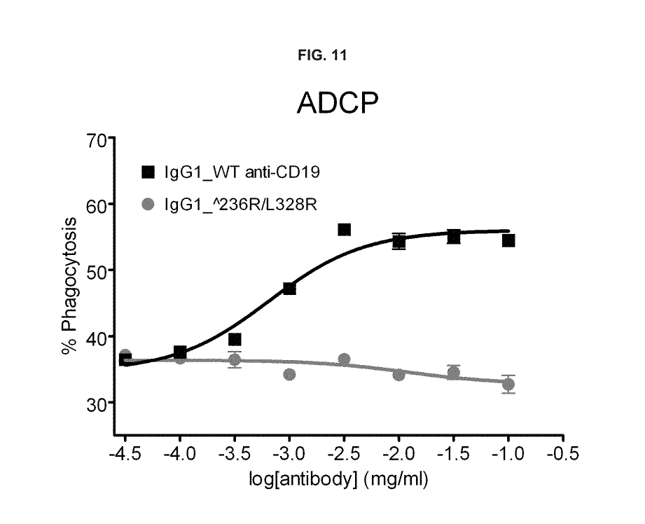

Phagocytosis carried out using the variable region of an anti-CD19 antibody, a humanized and affinity matured version of the murine 4G7 antibody as described U.S. patent application Ser. No. 11/838,824, titled “Optimized Antibodies that Target CD19,” filed Aug. 14, 2007. The heavy chain variable region of this antibody was subcloned into the pcDNA3.1 vector containing the heavy chain constant regions of IgG1 and ̂236R/L328R. Antibodies were expressed and purified as described above. CD14+ macrophages were purified from PBMCs by EasySep® Human Monocyte Enrichment Kit without CD16 depletion (Stemcell Technologies). Purified CD14+ monocytes were cultured in M-CSF (Peprotech) at 50 ng/ml for 5 days in a humidified incubator and differentiated into macrophages. Macrophage ADCP was determined by flow cytometry using CD19+Ramos cells as target cells. Target cells were labeled with PKH67 (Sigma) and seeded into 96-well plates in the presence of 10% human serum. Fc variant antibodies were diluted serially to half-log concentrations and added to the target cells such that the highest concentration was 1 jtg/ml. Monocyte-derived macrophages were then added at an effector to target ratio of 3:1, cells were spun down briefly, and incubated at 37° C. for 4 h. Cells were detached from the plate surface with HyQtase, stained with anti-CD11b APC, anti-CD14 APC, and anti-CD66 PE, washed with PBS, and fixed with 1% paraformaldehyde. Phagocytosis was evaluated on a FACS Canto II flow cytometer (BD Biosciences), and percent phagocytosis was calculated as the number of double positive cells divided by the total number of tumor cells. The intensity of CD66 staining was used to determine the degree to which tumor cells were internalized. FIG. 11 shows the results of the experiment. As can be seen, in contrast to WT IgG1, the variant, which contains an insertion in the hinge and a substitution in the Fc region, does not mediate ADCP.

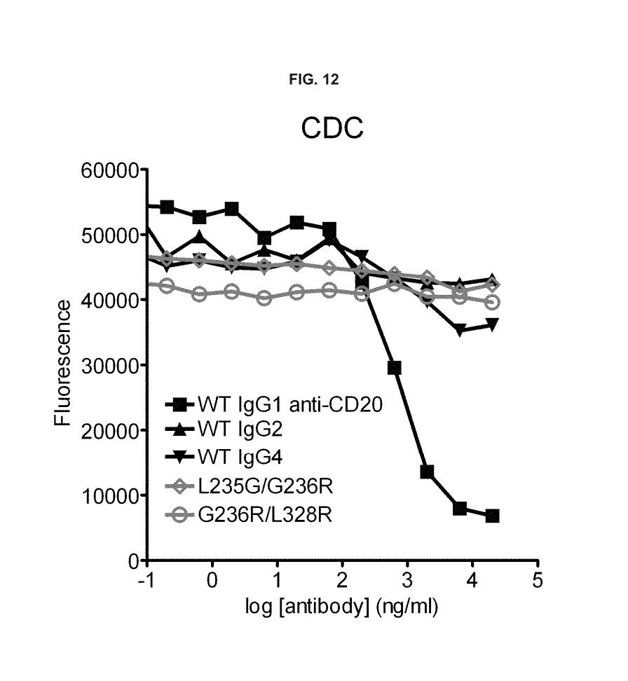

Finally, select variants with reduced FcγR binding were further tested for their capacity to mediate complement mediated cytotoxicity (CDC). The binding site for complement on the Fc region is separate from but overlapping with the site for binding to FcγRs. CDC activity was tested in the context of antibodies targeting CD20. The variants were constructed in the context of the anti-CD20 antibody PRO70769 (PCT/US2003/040426, hereby entirely incorporated by reference), which is known to mediate measurable CDC and ADCC in cell-based assays. The genes for the variable regions of PRO70769 were constructed using recursive PCR, and subcloned into the mammalian expression vector pcDNA3.1Zeo (Invitrogen) comprising the full length light kappa (CK) for the light chain, and either variant or WT IgG heavy chain constant regions. Antibodies were expressed and purified as described above. A cell-based assay was used to measure the capacity of the Fc variants to mediate CDC. Lysis was measured using release of Alamar Blue to monitor lysis of Fc variant and WT anti-CD20-opsonized WIL2-S lymphoma cells by human serum complement. Target cells were washed 3× in 10% FBS medium by centrifugation and resuspension, and WT or variant rituximab antibody was added at the indicated final concentrations. Human serum complement (Quidel) was diluted 50% with medium and added to antibody-opsonized target cells. Final complement concentration was ⅙^{th} original stock. Plates were incubated for 2 hrs at 37° C., Alamar Blue was added, cells were cultured for two days, and fluorescence was measured. Data from this assay are shown in FIG. 12. As can be seen, the variants with modifications at positions 235, 236, and 328, do not mediate CDC activity, similarly to WT IgG2 and IgG4 and in contrast to IgG1 anti-CD20.

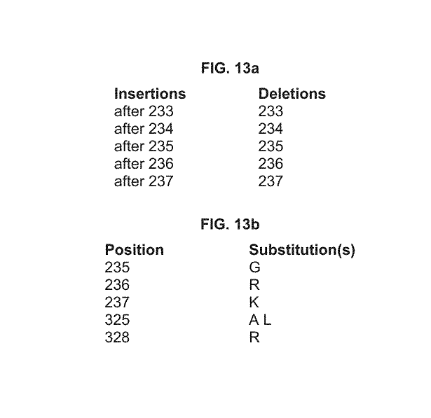

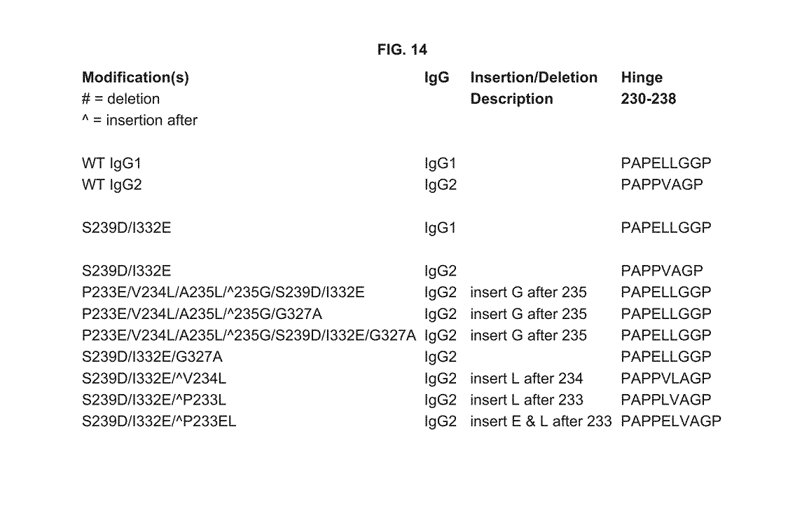

The results show that insertions and deletions in the hinge region, particularly at or after positions 233-237, provide the capability to reduce and even ablate FcγR- and complement-mediated effector functions. In addition, the data show that combination of insertions and deletions with substitutions in the Fc region are good. In particular, insertions and deletions in the hinge region may be combined preferrably with substitutions at positions 235, 236, 237, 325, and 328. For example, substitutions 235G, 236R, 237K, 325L, 325A, and 328R may be combined with insertions after positions 233, 234, 235, 236, and 237, and/or with deletions at positions 233, 234, 235, 236, and 237. Preferred embodiments of the invention for reducing or ablating FcγR- and/or complement-mediated effector function are provided in FIG. 13.

This list of preferred Fc variants is not meant to constrain the present invention. Because combinations of Fc variants of the present invention have typically resulted in additive or synergistic binding modulations, and accordingly additive or synergistic modulations in effector function, it is anticipated that as yet unexplored combinations of the Fc variants provided in the present invention, or with other previously disclosed modifications, will also provide favorable results. Indeed all combinations of the any of the insertions, deletions, and/or substitutions provided are embodiments of the present invention. Furthermore, combinations of any of the Fc variants of the present invention with other discovered or undiscovered Fc variants may also provide favorable properties, and these combinations are also contemplated as embodiments of the present invention. Further, insertions, deletions, and substitutions at all positions disclosed herein are contemplated.

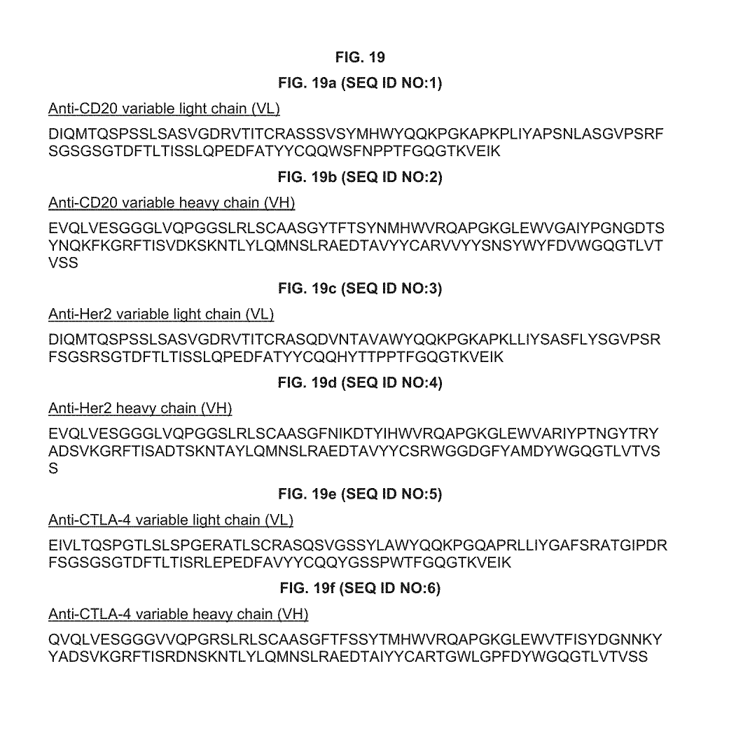

As discussed above, reduced FcγR affinity and/or effector function may be optimal for Fc polypeptides for which Fc ligand binding or effector function leads to toxicity and/or reduced efficacy. For example, antibodies that target CTLA-4 block inhibition of T-cell activation and are effective at promoting anti-tumor immune response, but destruction of T cells via antibody mediated effector functions may be counterproductive to mechanism of action and/or potentially toxic. Indeed toxicity has been observed with clinical use of the anti-CTLA-4 antibody ipilimumab (Maker et al., 2005, Ann Surg Oncol 12:1005-16, hereby entirely incorporated by reference). The sequences for the anti-CTLA-4 antibody ipilimumab (Mab 10D.1, MDX010) (U.S. Pat. No. 6,984,720, hereby entirely incorporated by reference) are provided in FIG. 19. The use of an anti-CTLA-4 here is solely an example, and is not meant to constrain application of the Fc variants to this antibody or any other particular Fc polypeptide. Other exemplary applications for reduced Fc ligand binding and/or effector function include but are not limited to anti-TNFct antibodies, including for example infliximab and adalimumab, anti-VEGF antibodies, including for example bevacizumab, anti-ct4-integrin antibodies, including, for example, natalizumab, and anti-CD32b antibodies, including, for example, those described in U.S. Ser. No. 10/643,857, hereby entirely incorporated by reference.

### Example 2. Fc Variants with Selective FcγR Affinity

Improvement in affinity for FcγRs is a goal for enhancing the therapeutic activity of antibodies that are used to treat cancers and infectious diseases. A potentially important parameter in this approach is the selectivity of an antibody variant for activating versus inhibiting receptors. Whereas NK cells only express the activating receptor FcγRIIIa, other potentially important immune cell types, including neutrophils, macrophages, and dendritic cells, express the inhibitory receptor FcγRIIb, as well the other activating receptors FcγRI and FcγRIIa. For these cell types optimal effector function may result from an antibody variant that has enhanced affinity for activation receptors, for example, FcγRI, FcγRIIa, and FcγRIIIa, yet reduced or unaltered affinity for the inhibitory receptor FcγRIIb. Notably, these other cells types can utilitize FcγRs to mediate not only innate effector functions that directly lyse cells, for example ADCC, but can also phagocytose targeted cells and process antigen for presentation to other immune cells, events that can ultimately lead to the generation of adaptive immune response. Yet because all FcγRs interact with the same binding site on Fc, and because of the high homology among the FcγRs, obtaining variants that selectively enhance or reduce FcγR affinity is a major challenge.

The data provided in FIG. 7 indicate that WT IgG2 has a favorable FcγRIIa:FcγRIIb profile, that is greater affinity for the activating receptor H131 and R131 FcγRIIa relative to the activating receptor FcγRIIb. However, WT IgG2 has poor binding to FcγRI and FcγRIIIa. Amino acid modifications were designed in an effort to engineer IgG2 such that it maintains its favorable FcγRIIa:FcγRIIb profile, but binds the other activating receptors FcγRI and FcγRIIIa with enhanced affinity. These variants, listed in FIG. 14, comprise insertions, deletions, and substitutions in the context of IgG2.

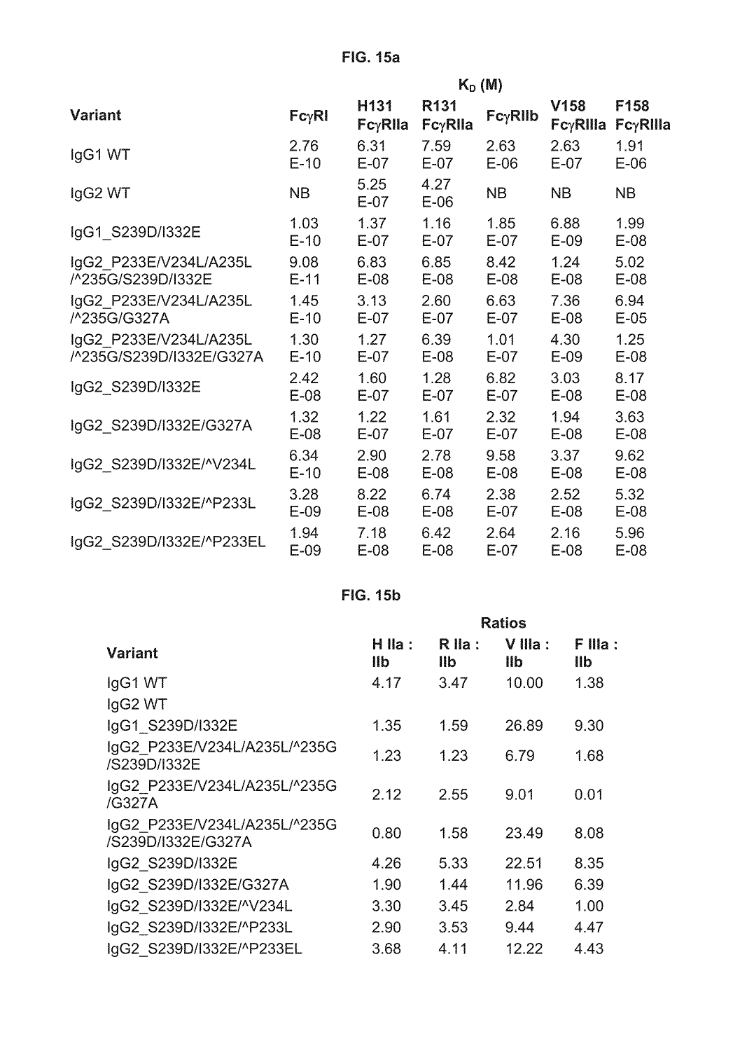

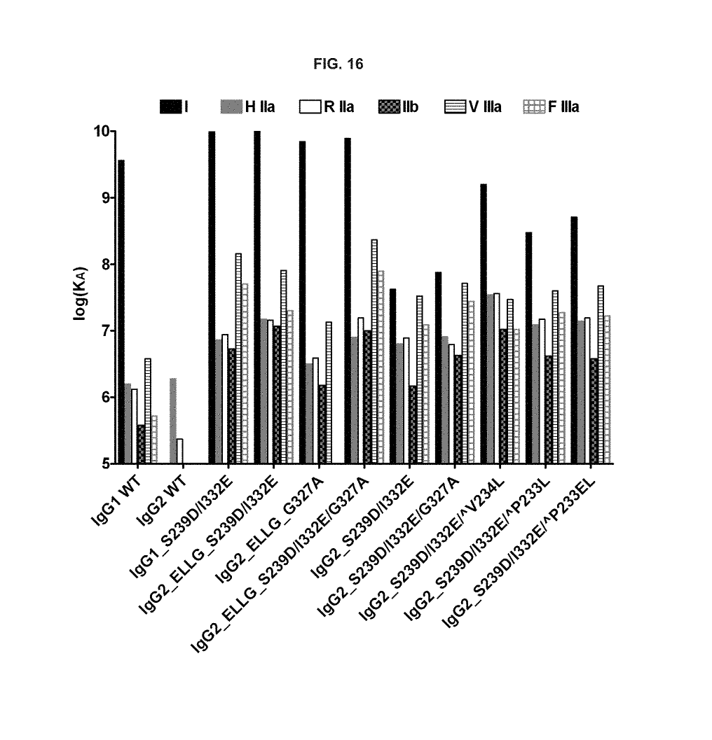

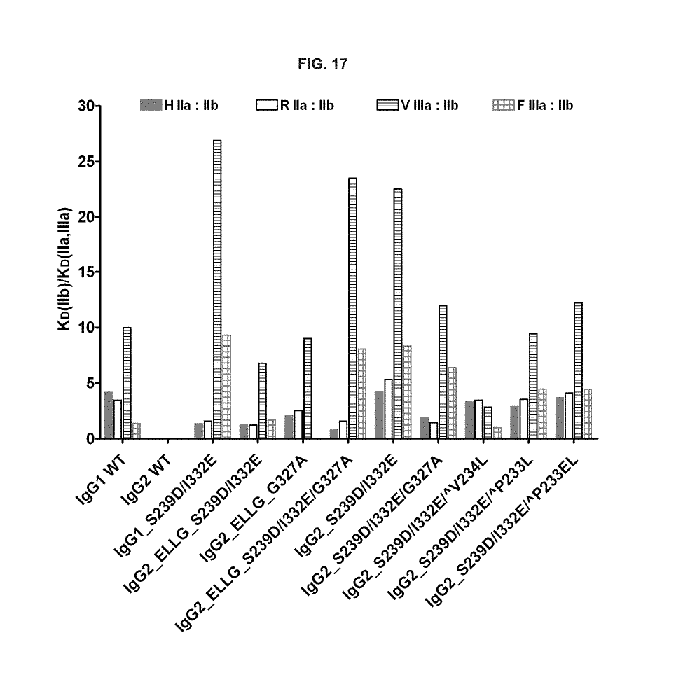

Variants were constructed in the context of the anti-Her2 antibody trastuzumab, expressed, and purified as described above. Binding affinity to the human FcγRs was determined by Biacore as described above. Global langmuir fits of the data provided the equilibrium dissociation constants (K_{D}s) (FIG. 15a). The fold affinities of the activating receptors FcγRIIa and FcγRIIIa (both isoforms of each) relative to the inhibitory receptor FcγRIIb are plotted in FIG. 15b. The log of the affinities and the ratio of activating to inhibitory receptors are plotted in FIG. 16 and FIG. 17, respectively. As can be seen, the insertions and deletions in the hinge region, as well as substitutions in the Fc region, can be used to control the affinities and selectivities of the different FcγRs

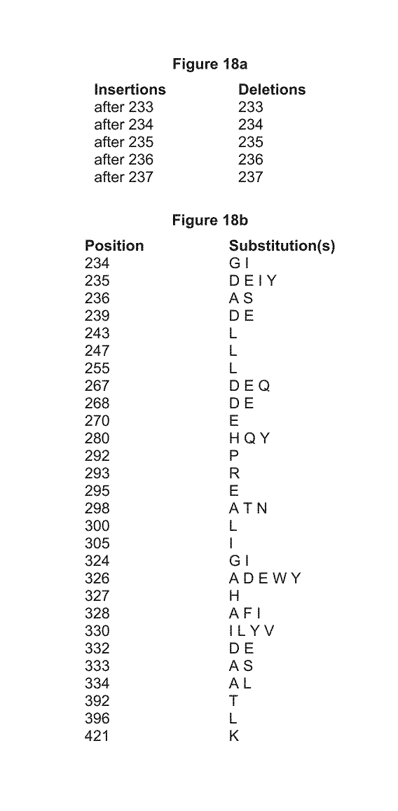

Taken together, the data provided in the present invention indicate that insertions and deletions in the hinge region may be used to modulate FcγR affinity and selectivity. In particular, insertions after positions 233, 234, 235, 236, and 237, and deletions at positions 233, 234, 235, 236, and 237 may provide optimal effector function properties. The current invention also demonstrates that combination of said amino acid modifications with other Fc substitutions may further provide optimal effector function properties. For example, substitutions that may be combined with the modifications of the invention are described in U.S. Ser. No. 10/672,280; U.S. Ser. No. 10/822,231; U.S. Ser. No. 11/396,495; U.S. Ser. No. 11/124,620; U.S. Ser. No. 11/538,406; U.S. Pat. No. 6,737,056; Shields et al, Journal of Biological Chemistry, 2001, 276(9):6591-6604; U.S. Pat. No. 6,528,624; Idusogie et al., 2001, J. Immunology 1 66:2571-2572; U.S. Ser. No. 10/754,922; U.S. Ser. No. 10/902,588; U.S. Ser. No. 10/370,749; Stavenhagen et al., 2007, Cancer Research 67(18):8882-90; all of which are herein expressly incorporated by reference. In a most preferred embodiment, the insertions and deletions of the invention are combined with one or more amino acid substitutions at a position selected from the group consisting of 234, 235, 236, 239, 243, 247, 255, 267, 268, 270, 280, 292, 293, 295, 298, 300, 305, 324, 326, 327, 328, 330, 332, 333, 334, 392, 396, and 421. For example, preferred substitutions that may be combined with the insertions and deletions of the invention include but are not limited to 234G, 234I, 235D, 235E, 235I, 235Y, 236A, 236S, 239D, 239E, 243L, 247L, 255L, 267D, 267E, 267Q, 268D, 268E, 270E, 280H, 280Q, 280Y, 292P, 293R, 295E, 298A, 298T, 298N, 300L, 305I, 324G, 324I, 326A, 326D, 326E, 326W, 326Y, 327H, 328A, 328F, 328I, 330I, 330L, 330Y, A330V, 32D, 332E, 333A, 333S, 334A, 334L, 392T, 396L, and 421K. Preferred combinations of insertions, deletions, and substitutions are described in FIG. 18.

### Example 3. Non-Naturally Occurring Modifications

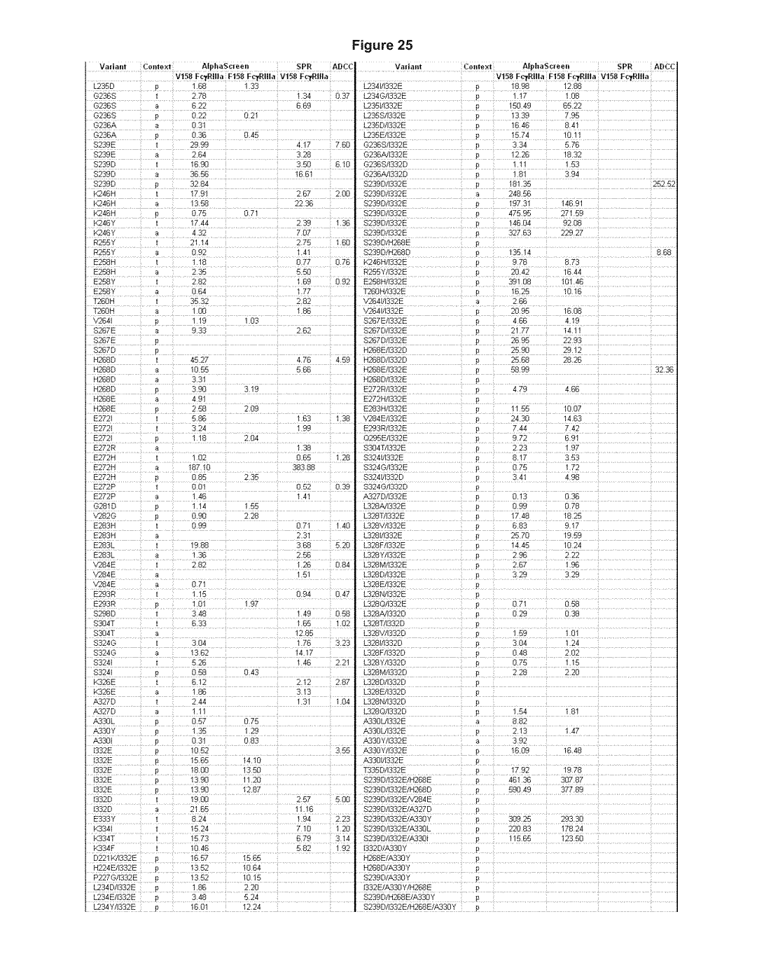

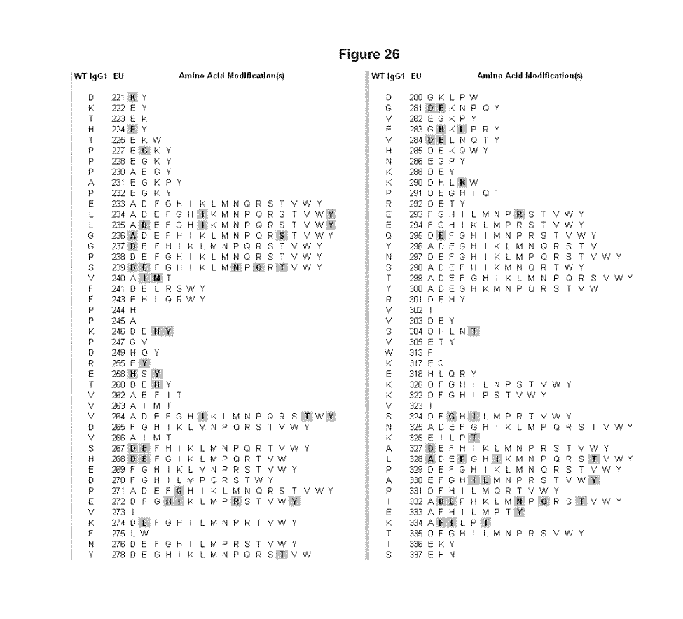

Novel Fc variants have been successfully engineered, primarily in the context of the IgG1 isotype, with selectively enhanced binding to FcγRs, and these variants have been shown to provide enhanced potency and efficacy in cell-based effector function assays (U.S. Ser. No. 10/672,280, U.S. Ser. No. 10/822,231, U.S. Ser. No. 60/627,774, U.S. Ser. No. 60/642,477, and U.S. Ser. No. 60/723,294, entitled “Optimized Fc Variants”, filed Oct. 3, 2005, all expressly incorporated by reference). FIGS. 24 and 25 summarize these variants and the data detailing their properties with respect to Fc ligand affinity and effector function. FIG. 26 summarizes the amino acid modifications that compose this set of variants.

The variants described in FIGS. 24-26 provide a variety of unique biological and clinical properties. A number of variants provide substantial enhancements in FcγR affinity, in particular to one or both isoforms (V158 and F158) of the activating receptor FcγRIIIa. For example substitutions at positions 239, 268, and 332 provide substantial improvements in FcγR binding and effector function. A number of variants have been obtained with altered specificities for the various Fc ligands. The selective affinity of a variant for the different FcγRs may be an important factor in determining the optimal therapeutic IgG. For example, the affinity of a variant for FcγRI, the relative affinity for FcγRIII versus FcγRIIb, and/or the relative affinity for FcγRIIa versus FcγRIIb may be important determinants of the capacity of an antibody or Fc fusion to mediate ADCC or ADCP, or elicit long-term immunity. For example, the balance between FcγRIIa and FcγRIIb establishes a threshold of DC activation and enables immune complexes to mediate opposing effects on dendritic cell (DC) maturation and function (Boruchov et al., 2005, J Clin Invest, September 15, 1-10). Thus, variants that selectively ligate FcγRIIa or FcγRIIb may affect DC processing, T cell priming and activation, antigen immunization, and/or efficacy against cancer (Dhodapkar & Dhodapkar, 2005, Proc Natl Acad Sci USA, 102, 6243-6244). Such variants may be employed as novel strategies for targeting antigens to the activating or inhibitory FcγRs on human DCs to generate either antigen-specific immunity or tolerance. Some variants provide selective enhancement in binding affinity to different Fc ligands, whereas other provide selective reduction in binding affinity to different Fc ligands. By “selective enhancement” as used herein is meant an improvement in or a greater improvement in binding affinity of a variant to one or more Fc ligands relative to one or more other Fc ligands. For example, for a given variant, the Fold WT for binding to, say FcγRIIa, may be greater than the Fold WT for binding to, say FcγRIIb. By “selective reduction” as used herein is meant a reduction in or a greater reduction in binding affinity of a variant to one or more Fc ligands relative to one or more other Fc ligands. For example, for a given variant, the Fold WT for binding to, say FcγR1, may be lower than the Fold WT for binding to, say FcγRIIb. As an example of such selectivity, G236S provides a selective enhancement to FcγRII's (IIa, IIb, and IIc) relative to FcγR1 and FcγRIIIa, with a somewhat greater enhancement to FcγRIIa relative to FcγRIIb and FcγRIIc. G236A, however, is highly selectively enhanced for FcγRIIa, not only with respect to FcγRI and FcγRIIIa, but also over FcγRIIb and FcγRIIc. Selective enhancements and reductions are observed for a number of Fc variants, including but not limited to variants comprising substitutions at EU positions 234, 235, 236, 267, 268, 292, 293, 295, 300, 324, 327, 328, 330, and 335. In particular, receptor selectivity may be provided by variants comprising one or more substitutions selected from the group consisting of 236S, 236A, 267D, 267E, 268D, 268E, 293R, 324I, 327D, 272R, 328A, 328F, 271G, 235Y, 327D, 328A, 328F, 324G, 330Y, 330L, and 330I. FIG. 26 highlights preferred non-naturally occurring modifications that provide optimized Fc ligand binding and/or effector function properties. Alternately preferred non-naturally occurring modifications include 234Y, 234I, 235Y, 235I, 235D, 236S, 237D, 239D, 239E, 239N, 239Q, 239T, 240M, 246H, 246Y, 255Y, 258Y, 264I, 264T, 264Y, 267D, 267E, 271G, 272Y, 272H, 272R, 272I, 274E, 278T, 283L, 283H, 293R, 324G, 324I, 326T, 327D, 328A, 328F, 328T, 330L, 330Y, 330I, 332D, 332E, 332N, 332Q, 332T, 333Y, 334F, and 334T. Most preferred non-naturally occurring modifications include 234Y, 234I, 235Y, 235I, 235D, 236S, 237D, 239D, 239E, 239N, 239Q, 239T, 264I, 264T, 264Y, 267D, 267E, 324G, 324I, 327D, 328A, 328F, 328T, 330L, 330Y, 330I, 332D, 332E, 332N, 332Q, and 332T.

### Example 4. IgG Variants with Non-Naturally Occurring Modifications

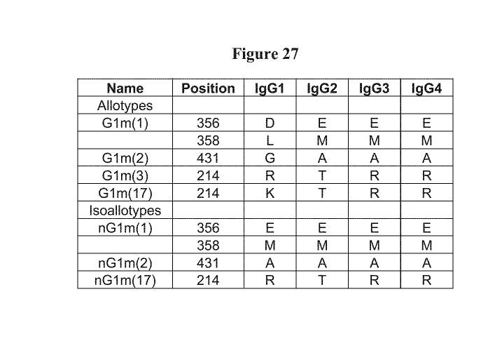

The present invention provides immunoglobulins wherein the aforedescribed novel variants are utilized in the context of alternate IgG isotypes. FIG. 1 shows the sequences of the four IgG isotypes IgG1, IgG2, IgG3, and IgG4, with differences from IgG1 highlighted. Thus, FIG. 1 provides the isotypic differences between the four IgGs. For completeness, it is noted that in addition to isotypic differences, a number of immunoglobulin polmorphisms (referred to as Gm polymorphisms) or allotypes exist in the human population. Gm polymorphism is determined by the IGHG1, IGHG2 and IGHG3 genes which have alleles encoding allotypic antigenic determinants referred to as G1m, G2m, and G3m allotypes for markers of the human IgG1, IgG2 and IgG3 molecules (no Gm allotypes have been found on the gamma 4 chain) (Clark, 1997, IgG effector mechanisms, Chem Immunol. 65:88-110; Gorman & Clark, 1990, Semin Immunol 2(6):457-66). Allelic forms of human immunoglobulins have been well-characterized (WHO Review of the notation for the allotypic and related markers of human immunoglobulins. J Immunogen 1976, 3:357-362; WHO Review of the notation for the allotypic and related markers of human immunoglobulins. 1976, Eur. J. Immunol. 6, 599-601; Loghem E van, 1986, Allotypic markers, Monogr Allergy 19: 40-51). At present, 18 Gm allotypes are known: G1m (1, 2, 3, 17) or G1m (a, x, f, z), G2m (23) or G2m (n), G3m (5, 6, 10, 11, 13, 14, 15, 16, 21, 24, 26, 27, 28) or G3m (b1, c3, b5, b0, b3, b4, s, t, g1, c5, u, v, g5) (Lefranc, et al., The human IgG subclasses: molecular analysis of structure, function and regulation. Pergamon, Oxford, pp. 43-78 (1990); Lefranc, G. et al., 1979, Hum. Genet.: 50, 199-211). Additionally, other polymorphisms have been characterized (Kim et al., 2001, J. Mol. Evol. 54:1-9). As an example, FIG. 27 shows the allotypes and isoallotypes of the gamma1 chain of human IgG1 showing the positions and the relevant amino acid substitutions.

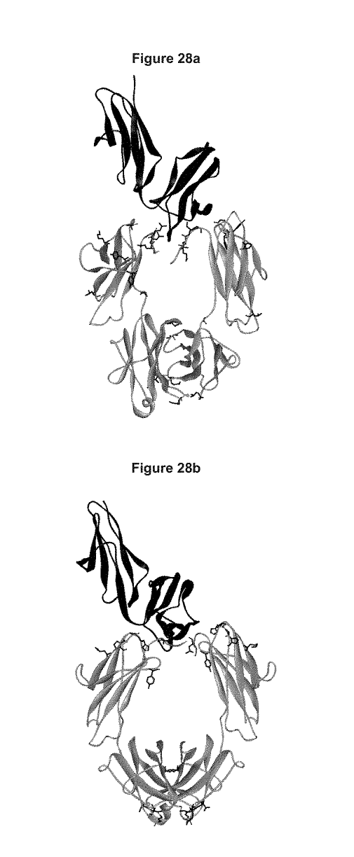

The different IgG isotypes offer a variety of unique physical, biological, and therapeutic properties. For example there are significant differences in stability, solubility, FcγR-mediated effector functions, complement-mediated effector functions, in vivo pharmacokinetics, and oligomerization state among the isotypes IgG1, IgG2, IgG3, and IgG4. These differences must be due to one or more of the isotypic differences between the IgGs shown in FIG. 1. For example, because the binding site for FcγRs resides on the Fc region, it is likely that the IgG differences in Fc, and even more likely the lower hinge and the CH2 domain, are responsible for the differences in their FcγR-mediated effector functions. FIGS. 28a and 28b highlight the differences between the Fc region of IgG1 and those of IgG2 and IgG4 respectively, mapped in the context of the IgG1 Fc/FcγRIIIb complex (pdb accession code 1E4K)(Sondermann et al., 2000, Nature 406:267-273).

In order to explore the properties of the different IgG isotypes, a matched set of IgG1, IgG2, and IgG4 antibodies were constructed with the variable region of the anti-Her2/neu antibody trastuzumab (Herceptin®, a registered trademark of Genentech, currently approved for treatment of breast cancer). The genes for the variable regions of trastuzumab were constructed using recursive PCR, and subcloned into the mammalian expression vector pcDNA3.1Zeo (Invitrogen) comprising the full length light kappa (Cκ) and heavy chain IgG1 constant regions. DNA was sequenced to confirm the fidelity of the sequences. Plasmids containing heavy chain gene (VH-Cγ1-Cγ2-Cγ3) (wild-type or variants) were co-transfected with plasmid containing light chain gene (VL-Cκ) into 293T cells. Media were harvested 5 days after transfection, and antibodies were purified from the supernatant using protein A affinity chromatography (Pierce). Antibody concentrations were determined by bicinchoninic acid (BCA) assay (Pierce).

In order to screen for FcγR binding, the extracellular region of human V158 FcγRIIIa was expressed and purified. The extracellular region of this receptor was obtained by PCR from a clone obtained from the Mammalian Gene Collection (MGC:22630). The receptor was fused at the C-terminus with a 6×His-tag and a GST-tag, and subcloned into pcDNA3.1zeo. Vector containing receptor was transfected into 293T cells, media were harvested, and receptors were purified using Nickel affinity chromatography. Receptor concentrations were determined by bicinchoninic acid (BCA) assay (Pierce). Binding affinity to human FcγRIIIa by the antibodies was measured using a quantitative and extremely sensitive method, AlphaScreen™ assay. The AlphaScreen is a bead-based luminescent proximity assay. Laser excitation of a donor bead excites oxygen, which if sufficiently close to the acceptor bead will generate a cascade of chemiluminescent events, ultimately leading to fluorescence emission at 520-620 nm. The AlphaScreen was applied as a competition assay for screening the antibodies. Commercial IgG was biotinylated by standard methods for attachment to streptavidin donor beads, and tagged human FcγRIIIa (V158 isoform) was bound to glutathione chelate acceptor beads. In the absence of competing antibody, antibody and FcγR interact and produce a signal at 520-620 nm. Addition of untagged antibody competes with the Fc/FcγR interaction, reducing fluorescence quantitatively to enable determination of relative binding affinities.

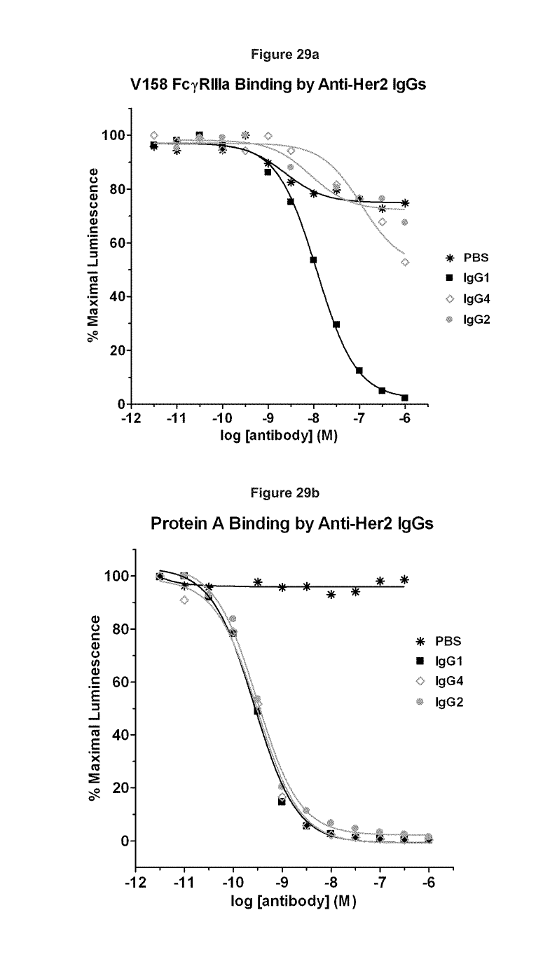

FIG. 29a presents the competition AlphaScreen binding data for binding of trastuzumab IgGs to human V158 FcγRIIIa. The binding data were normalized to the maximum and minimum luminescence signal provided by the baselines at low and high concentrations of competitor antibody respectively. The data were fit to a one site competition model using nonlinear regression, and these fits are represented by the curves in the figure. The results show that the FcγR-mediated effector functions are substantially greater for IgG1 than for IgG2 and IgG4, consistent with prior studies (Michaelsen et al., 1992, Molecular Immunology, 29(3): 319-326). FIG. 29b presents competition AlphaScreen data for binding of the IgGs to protein A, carried out using commercial protein A-conjugated acceptor beads. The data show that all of the variants bind comparably to protein A, indicating that the FcγR-affinity differences are not due to differences in stability, solubility, or other properties between the IgG isotypes.

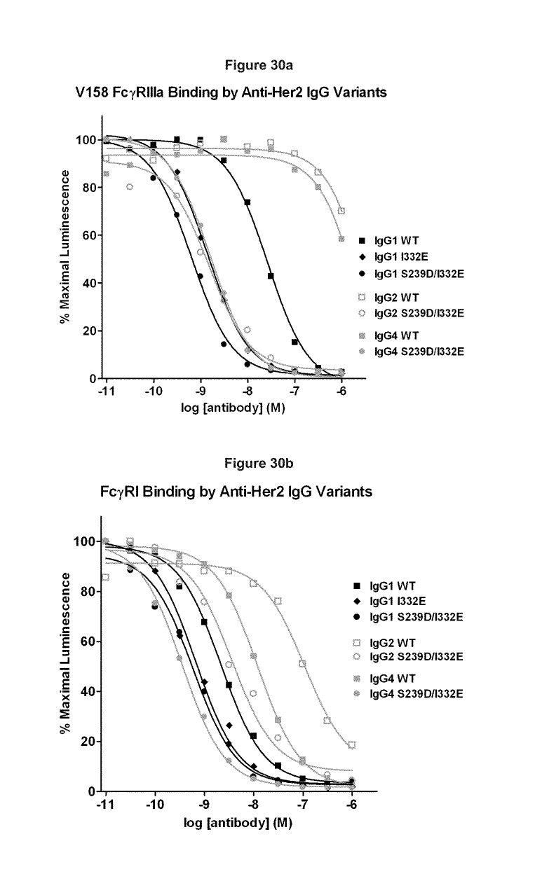

Non-naturally occurring modifications were constructed in the context of all three antibody isotypes. The substitutions S239D and I332E were introduced into the heavy chains of the trastuzumab IgG1, IgG2, and IgG4 antibodies using quick-change mutagenesis techniques (Stratagene), and antibodies were expressed and purified as described above. Competition AlphaScreen data were acquired as described above for binding to human V158 FcγRIIIa, as well as human FcγRI, which was constructed using recursive PCR and expressed and purified as described above. FIGS. 30a and 30b show the data for binding of the IgG variants to these receptors. The results show that the novel modifications S239D/I332E provide enhanced receptor binding to all three isotypes, despite the poor FcγR affinity of IgG2 and IgG4 relative to IgG1.

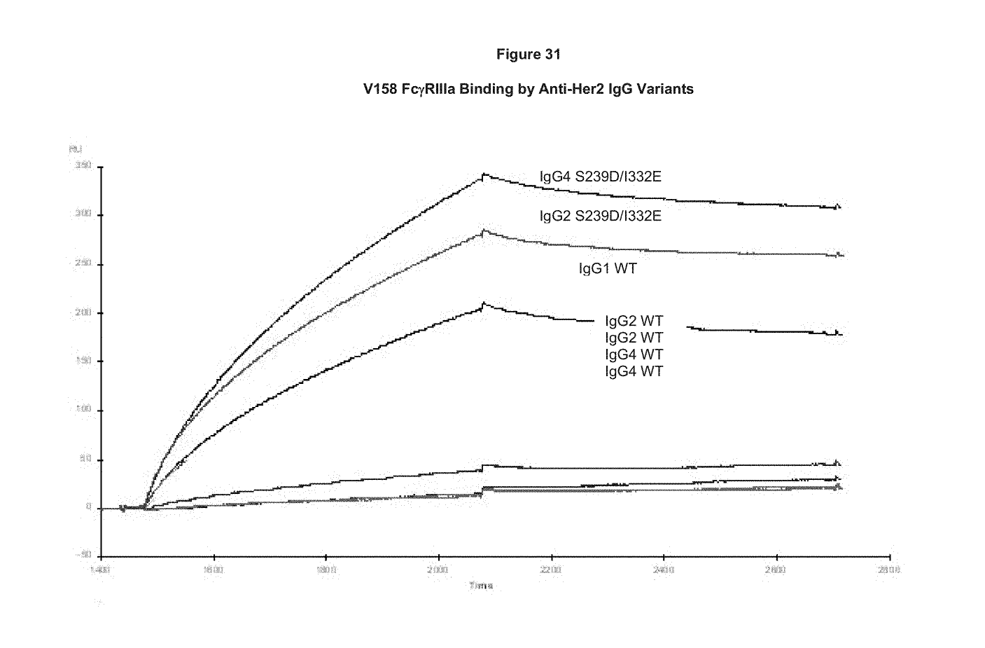

Surface Plasmon Resonance (SPR) (Biacore, Uppsala, Sweden) was carried out to further investigate the FcγRIIIa affinity of the IgG variants. Protein A (Pierce) was covalently coupled to a CMS sensor chip using NHS/EDC chemistry. WT or variant trastuzumab antibody was bound to the protein A CMS chip, and FcγRIIIa-His-GST analyte, in serial dilutions was injected (association phase) and washed (dissociation phase). Response in resonance units (RU) was acquired, and data were normalized for baseline response, obtained from a cycle with antibody and buffer alone. FIG. 31 provides the kinetic traces for the binding of WT IgG1, WT IgG2, WT IgG4, S239D/I332E IgG2, and S239D/I332E IgG4 antibodies to human V158 FcγRIIIa. The relative amplitudes of the binding traces reflect the relative FcγR affinities of the variants. The data corroborate the AlphaScreen data, indicating further that the novel modifications provide significant FcγR binding enhancements to IgG2 and IgG4.

### Example 5. IgGs Variants with Novel and Isotypic Amino Acid Modifications

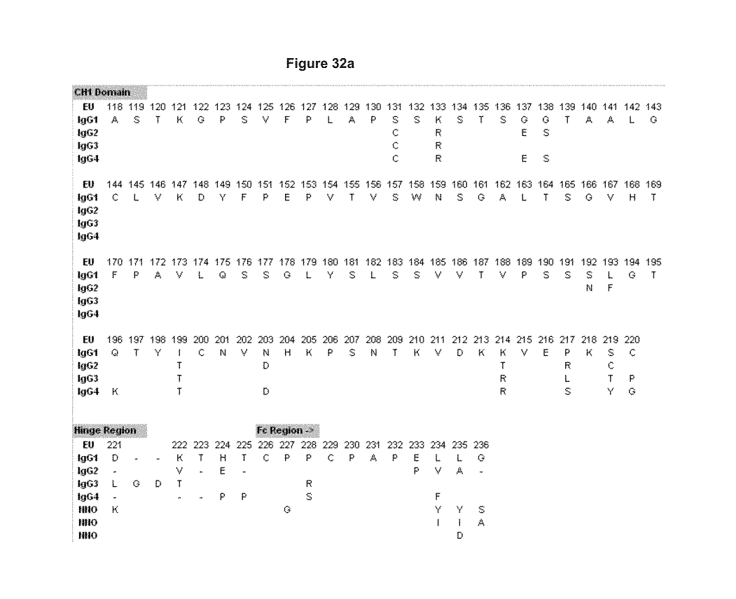

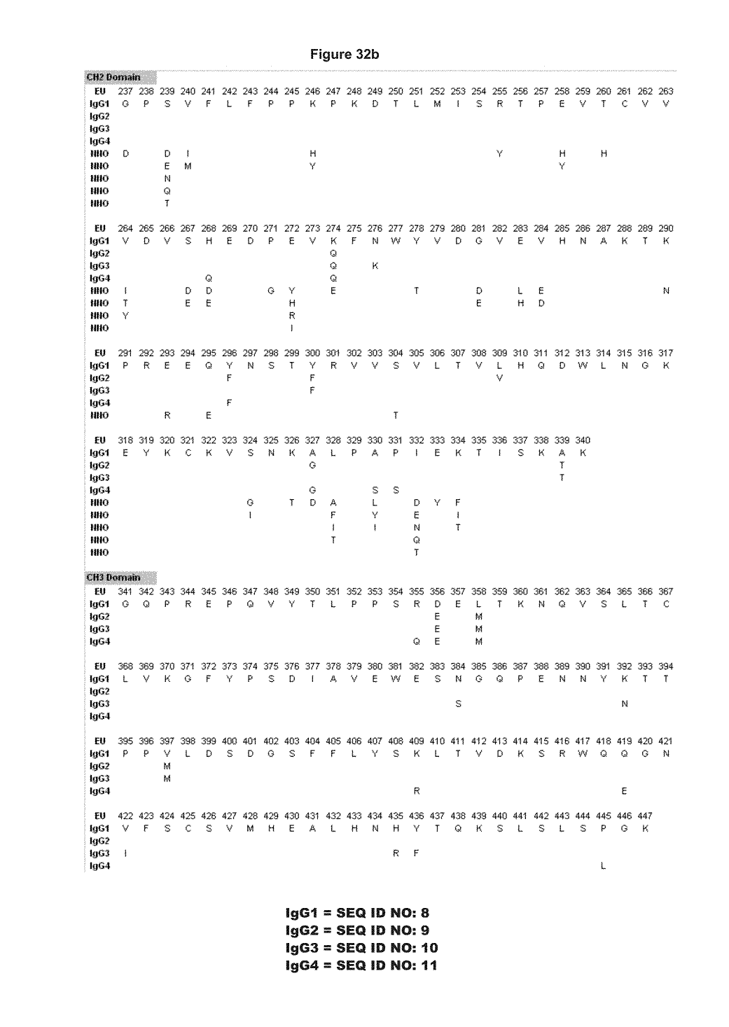

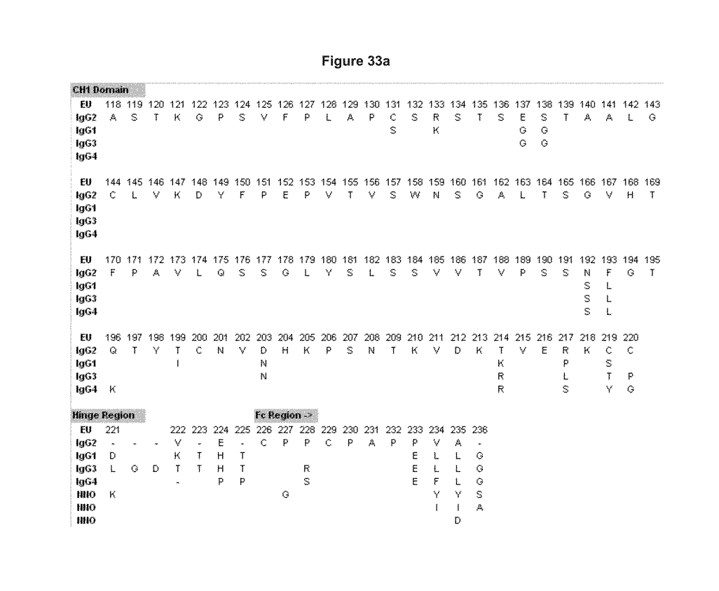

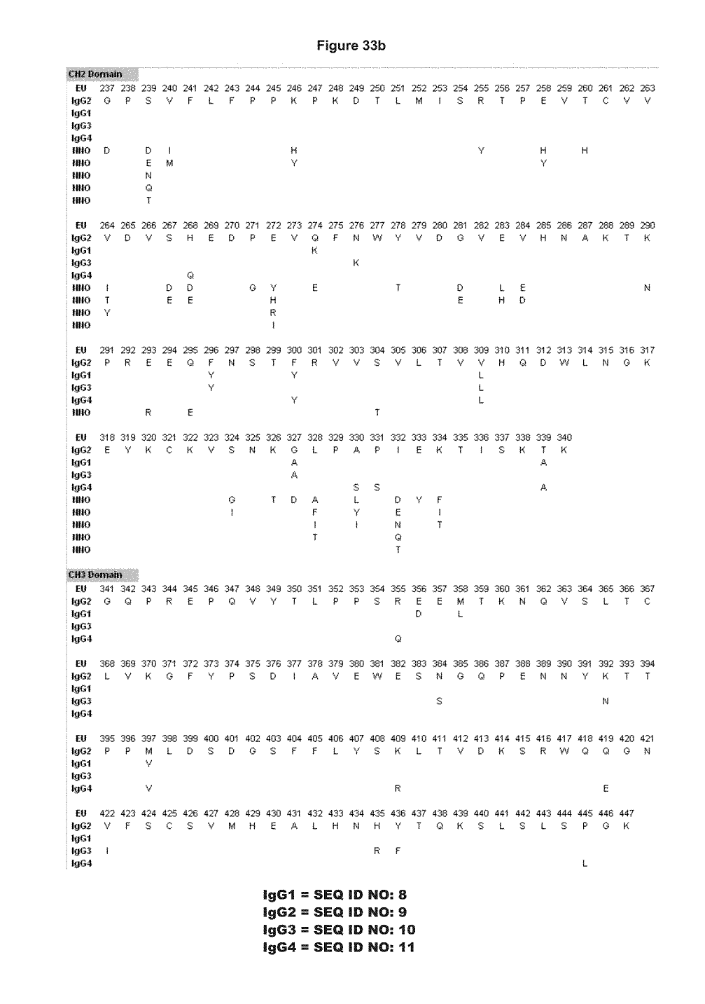

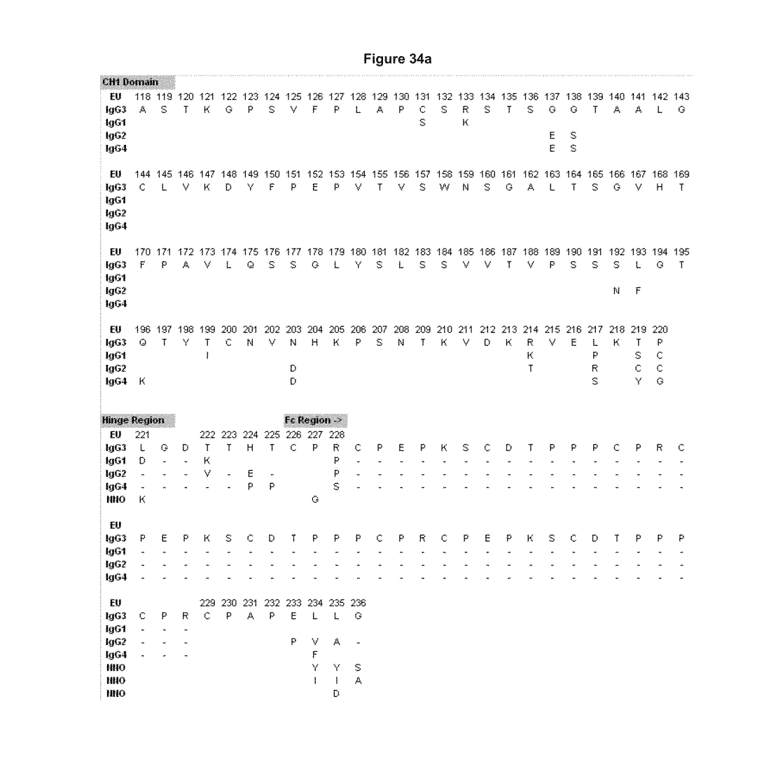

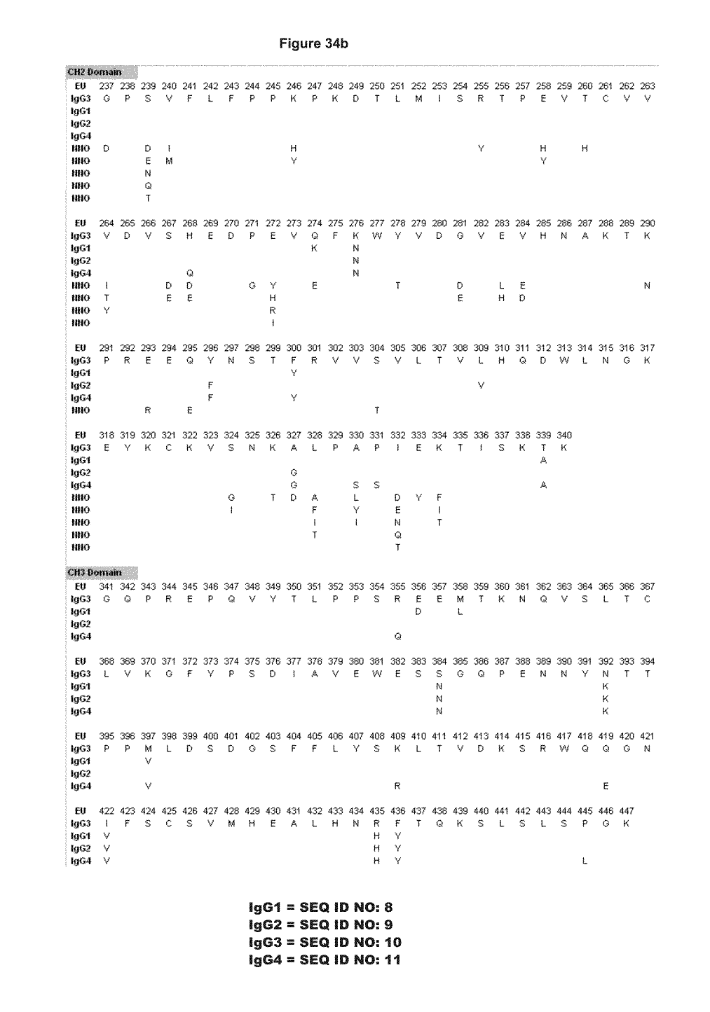

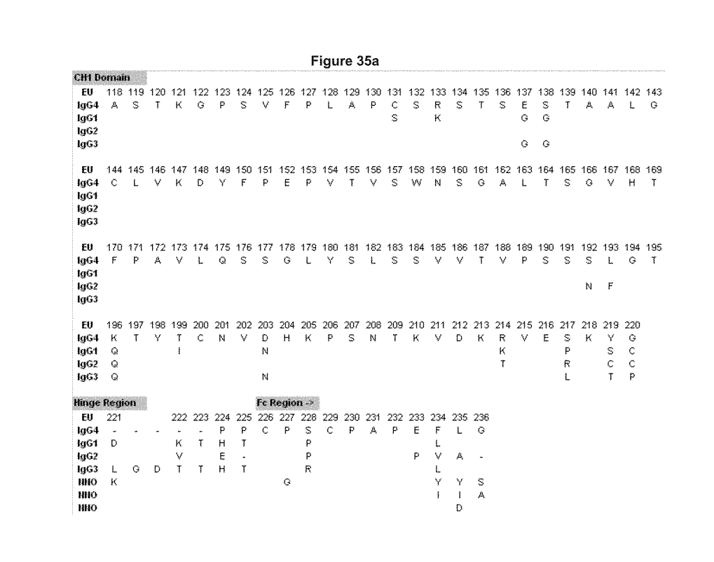

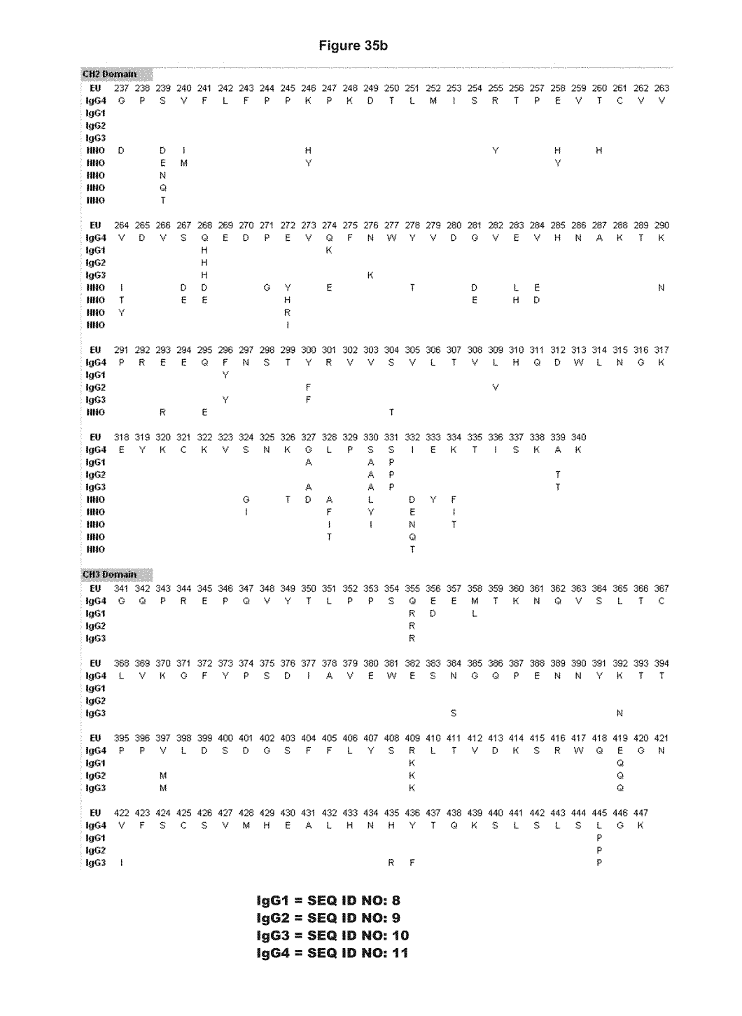

The present invention provides immunoglobulins wherein the aforedescribed novel variants are coupled with isotypic modifications to provide IgG variants with optimized properties. FIGS. 32-35 describe a set of novel and isotypic amino acid modifications for each isotype IgG1 (FIG. 32), IgG2 (FIG. 33), IgG3 (FIG. 34), and IgG4 (FIG. 35). The sequence of the parent IgG is provided explicitly, and novel and isotypic residues are provided at appropriate EU positions according to FIG. 26. As an example in FIG. 33, IgG2 is the parent immunoglobulin and comprises a deletion at EU position 236. IgG1, IgG2, and IgG3 all comprise glycines at position 236, and serine and alanine are two preferred novel substitutions at position 236. Thus, FIG. 33 describes in the parent immunoglobulin IgG2 the isotypic modifications -236G and the novel modifications—236S and -236A. According to FIGS. 33 and 26, the full set of novel modifications in the parent IgG2 at position 236 include -236A, -236D, -236E, -236F, -236H, -236I, -236K, -236L, -236M, -236N, -236P, -236Q, -236R, -236S, -236T, -236V, -236W, and -236Y.

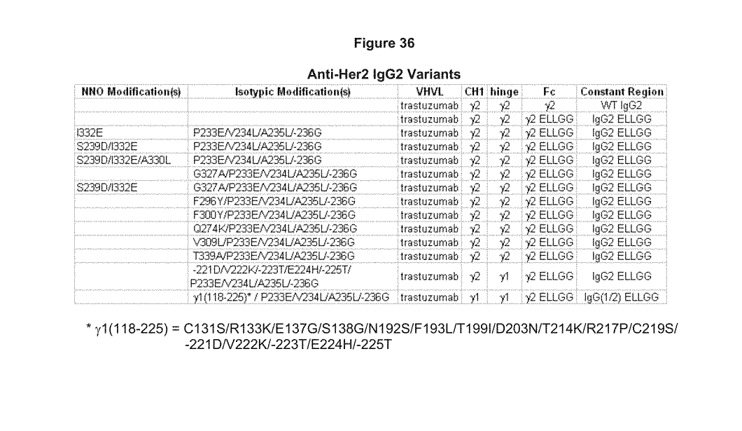

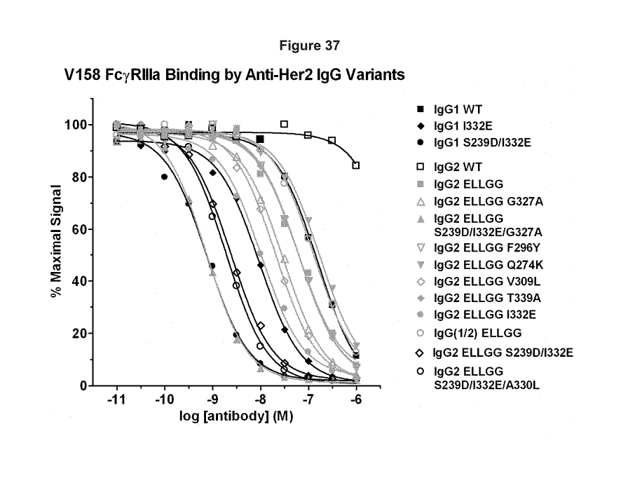

A set of IgG2 trastuzumab variants were constructed comprising novel and isotypic modifications using the information provided in FIG. 33. FIG. 36 provides this set of IgG variants. For simplicity, constant regions are labeled for easy reference. P233E/V234L/A235L/-236G IgG2, referred to as IgG2 ELLGG, is an IgG2 variant described previously (Chappel et al., 199I, Proc. Natl. Acad. Sci. USA 88(20):9036-9040; Chappel et al., 1993, Journal of Biological Chemistry 268:25124-25131). γ1(118-225)/P233E/V234L/A235L/-236G IgG2, referred to as IgG(1/2) ELLGG, is a novel IgG2 variant comprising the P233E/V234L/A235L/-236G modifications of IgG2 ELLGG and the full set of IgG2 to IgG1 isotypic modifications in the CH1 domain and hinge region (γ1(118-225)). These variants were constructed, expressed, and purified as described previously. FIG. 37 shows competition AlphaScreen data for binding of the IgG2 trastuzumab variants to human V158 FcγRIIIa, carried out as described. The results show the favorable FcγR binding properties of the IgG2 ELLGG and IgG(1/2) ELLGG variants. Furthermore, the results show that a number of novel and isotypic modifications significantly improve the FcγR binding affinity of the IgG2 isotype.

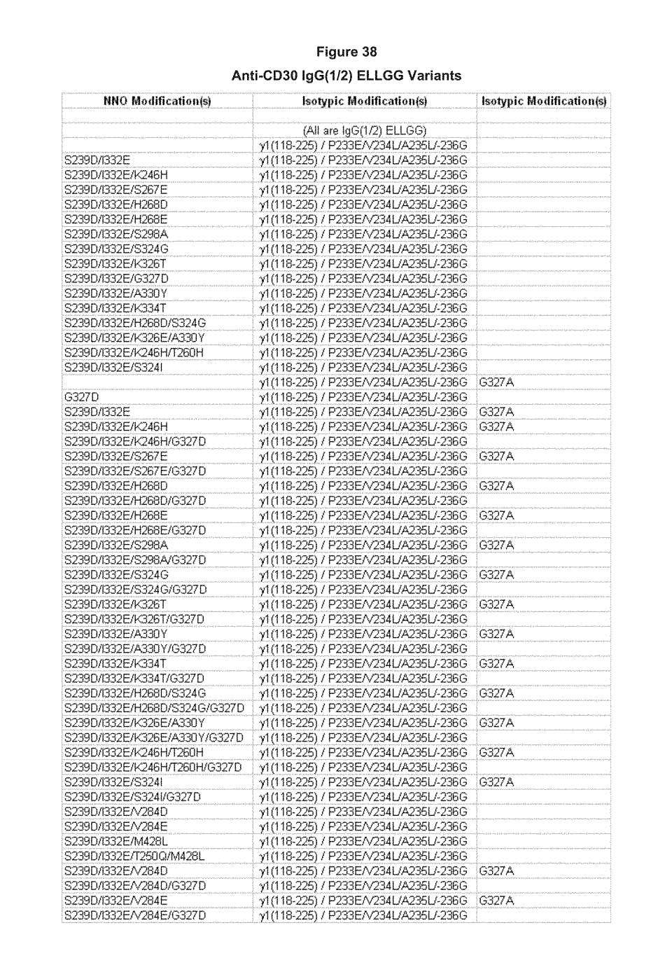

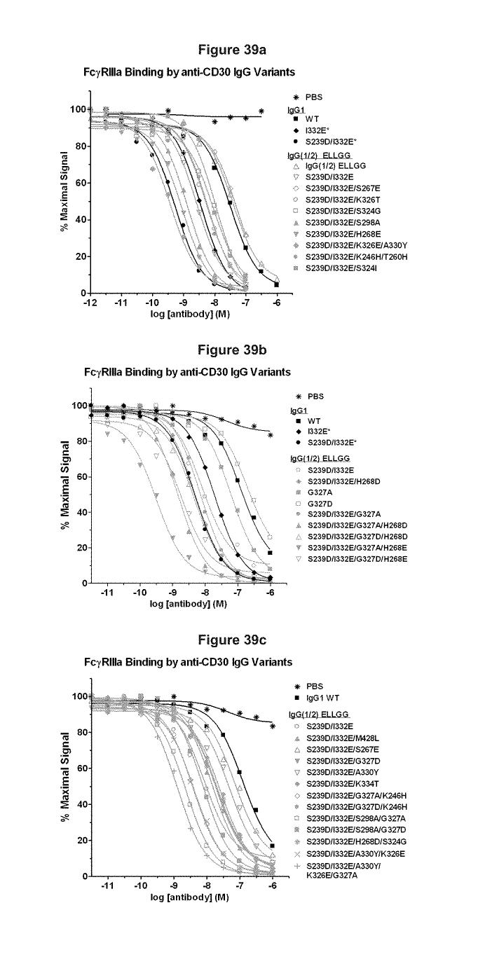

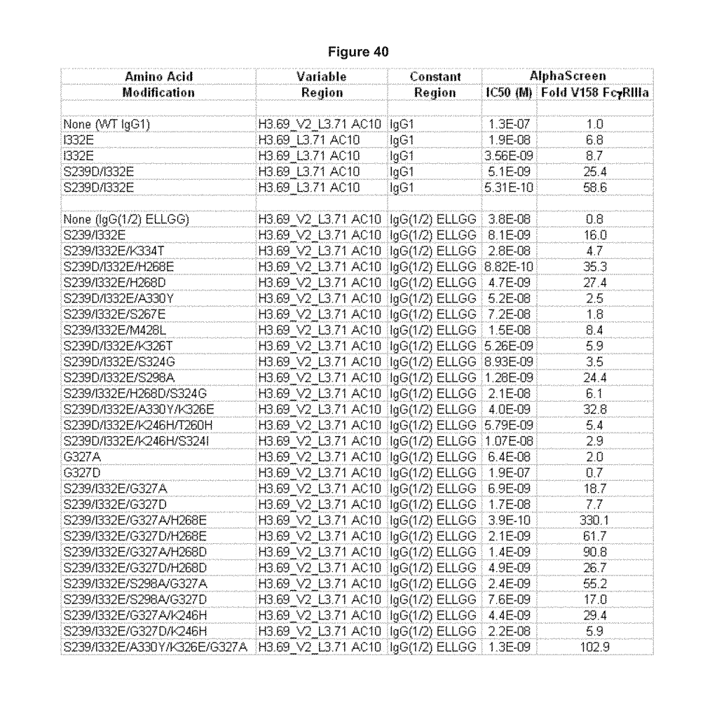

A series of isotypic and novel modifications were made and tested in the context of IgG(1/2) ELLGG to further explore the properties of this IgG variant. These variants are provided in FIG. 38. The variable region of these IgG variants is that of H3.69_V2_L3.69 AC10, which is an anti-CD30 antibody with reduced immunogenicity. H3.69_V2_L3.69 AC10 is a variant of H3.69_L3.71 AC10 described in U.S. Ser. No. 11/004,590 (herein expressly incorporated by reference) with a mutation I2V in the H3.69 VH region. The set of variants in FIG. 38 comprise novel and isotypic modifications in the context of IgG(1/2) ELLGG. These variants were constructed, expressed, and purified as described previously. FIG. 39 shows competition AlphaScreen data for binding of the anti-CD30 IgG2 variants to human V158 FcγRIIIa, carried out as described. The fits to the data provide the inhibitory concentration 50% (IC50) (i.e., the concentration required for 50% inhibition) for each antibody, thus enabling the relative binding affinities of Fc variants to be quantitatively determined. By dividing the IC50 for each variant by that of H3.69_V2_L3.71 AC10 IgG1, the fold-enhancement or reduction in receptor binding (Fold V158 FcγRIIIa) are obtained. These values are provided in FIG. 40. The results further show that the Fc ligand binding properties of the IgG isotypes can be significantly improved via engineering of novel and isotypic amino acid modifications.

Cell-based ADCC assays were carried out on the anti-CD30 IgG variants to investigate their effector function properties. ADCC was measured using either the DELFIA® EuTDA-based cytotoxicity assay (Perkin Elmer) or LDH Cytotoxicity Detection Kit (Roche Diagnostic Corporation, Indianapolis, Ind.). Human PBMCs were purified from leukopacks using a ficoll gradient. For europium-based detection, target cells were first loaded with BATDA at 1×106 cells/ml and washed 4 times. For both europium- and LDH-based detection, CD30+L540 Hodgkin's lymphoma target cells were seeded into 96-well plates at 10,000 cells/well, and opsonized using Fc variant or WT antibodies at the indicated final concentration. Triton X100 and PBMCs alone were typically run as controls. Effector cells were added at 25:1 PBMC5:target cells, and the plate was incubated at 37° C. for 4 hrs. Cells were incubated with either Eu3+ solution or LDH reaction mixture, and relative fluorescence units were measured. Data were normalized to maximal (triton) and minimal (PBMCs alone) lysis, and fit to a sigmoidal dose-response model using nonlinear regression. FIG. 41a-41d provide these data. The results show that the optimized FcγR binding properties of the IgG variants result in improved effector function.

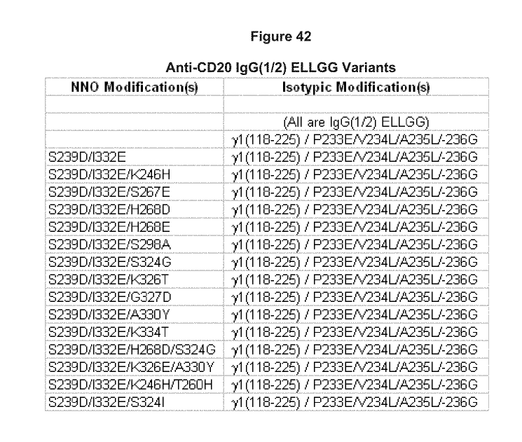

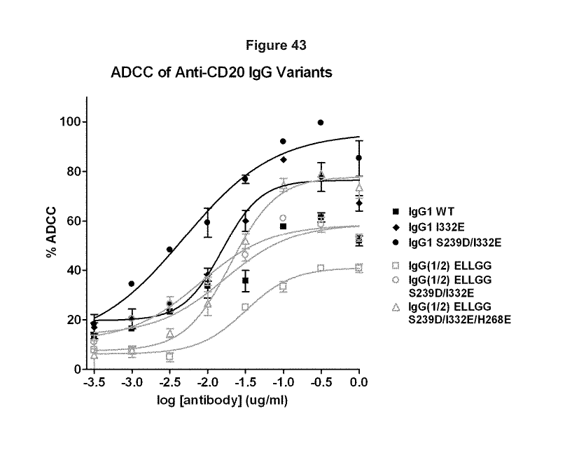

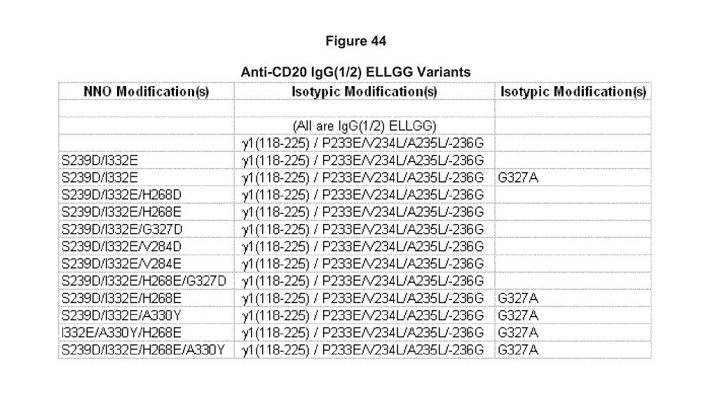

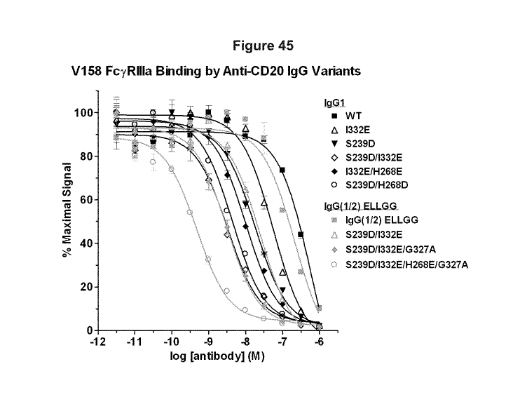

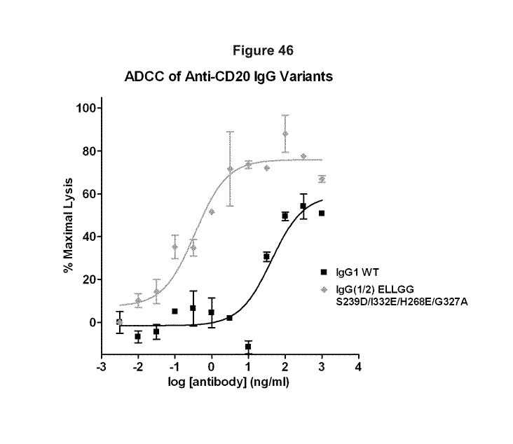

A set of IgG variants comprising novel and isotypic modifications were made and tested in the context of two antibodies that target the B-cell antigen CD20. FIG. 42 provides a set of IgG variants comprising the variable region of C2B8, an anti-CD20 antibody currently marketed as the biotherapeutic rituximab (U.S. Pat. No. 5,736,137). These variants were constructed, expressed, and purified as described previously. FIG. 43 shows cell-based ADCC data for select rituximab IgG2 variants against CD20+WIL2-S lymphoma target cells. FIG. 44 provides a set of IgG variants comprising the variable region of the anti-CD20 antibody PRO70769 (PCT/US2003/040426). These variants were constructed, expressed, and purified as described previously. FIG. 45 shows competition AlphaScreen data for binding of these anti-CD20 IgG variants to human V158 FcγRIIIa, and FIG. 46 provides a cell-based ADCC for one of the PRO70769 IgG variants against WIL2-S cells. The results are consistent with the aforedescribed results, indicating that the IgG variants are the invention are broadly applicable for improving clinically relevant antibodies.

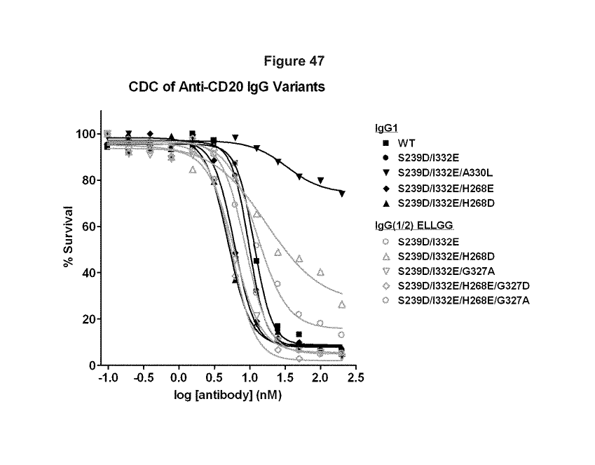

To explore the effect of the novel and isotypic modifications on complement activity, a cell-based CDC assay was performed. Target WIL2-S lymphoma cells were washed 3× in 10% FBS medium by centrifugation and resuspension, and seeded at 50,000 cells/well. Anti-CD20 antibodies was added at the indicated final concentrations. Human serum complement (Quidel, San Diego, Calif.) was diluted 50% with medium and added to antibody-opsonized target cells. Final complement concentration was approximately ⅙^{th} original stock. Plates were incubated for 2 hrs at 37° C., Alamar Blue was added, and cells were cultured for two days. Fluorescence was measured, and data were normalized to the maximum and minimum signal and fit to a sigmoidal dose-response curve. FIG. 47 shows these data. The results indicate that the novel and isotypic modifications of the invention can be further employed to modulate IgG CDC activity.

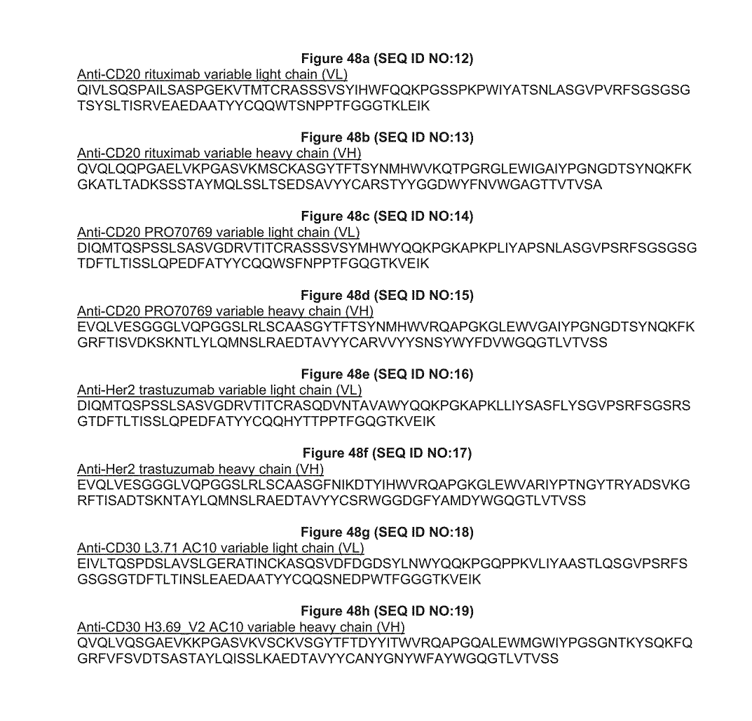

FIG. 48 provides the amino acid sequences of the variable region VL and VH domains utilized in the present invention, including the anti-CD20, anti-Her2, and anti-CD30 antibodies. These sequences are not meant to constrain the present invention to these variable regions. The present invention contemplates application of the described IgG variants to other antibodies that target CD20, Her2, and CD30. Particularly preferred are anti-CD20 antibodies that bind to an identical or overlapping CD20 epitope as C2B8, anti-CD20 antibodies that bind to an identical or overlapping CD20 epitope as PRO70769, anti-Her2 antibodies that bind to an identical or overlapping Her2 epitope as trastuzumab, and anti-CD30 antibodies that bind to an identical or overlapping CD30 epitope as H3.69_V2_L3.71 AC10. The present invention of course contemplates application of the described IgG variants to antibodies that target other antigens besides CD20, Her2, and CD30.

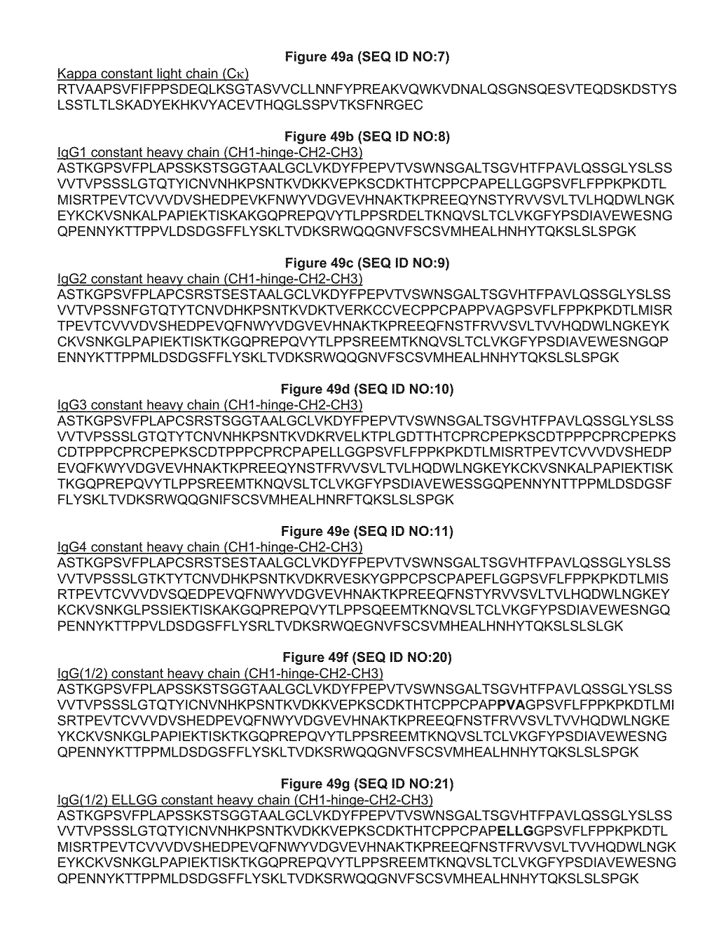

FIG. 49 provides the constant region amino acid sequences described in the present invention. These include the constant light chain kappa region, the four IgG isotypes IgG1, IgG2, IgG3, and IgG4, the IgG2 ELLGG constant region, and the IgG(1/2) ELLGG constant region. These sequences are not meant to constrain the present invention to these constant regions. For example, although the kappa constant chain (Cκ) was used in the present study, the lambda constant chain (Cλ) may be employed.

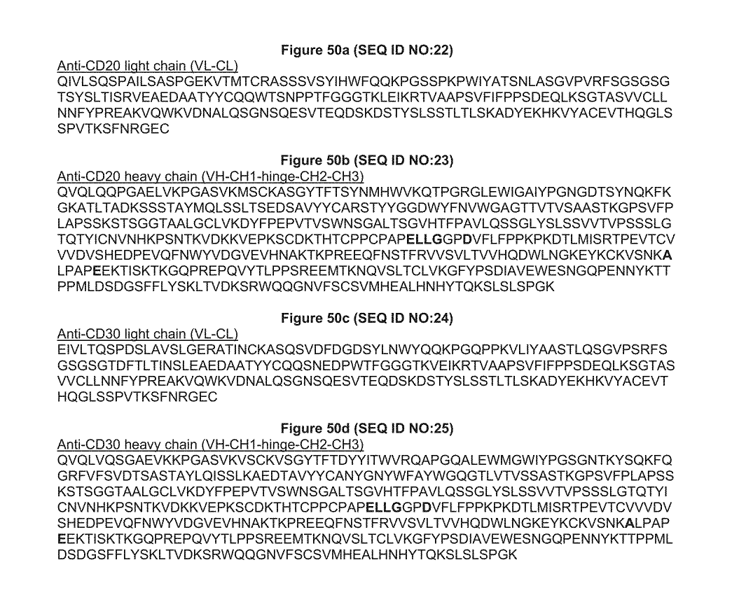

FIGS. 50a and 50b provide the amino acid sequences of the full length light and heavy chains of one of the anti-CD20 IgG variants described in the present invention. FIGS. 50c and 50d provide the amino acid sequences of the full length light and heavy chains of one of the anti-CD30 IgG variant described in the present invention.

### Example 6. Design of Fc Variants with Selective FcγR Affinity

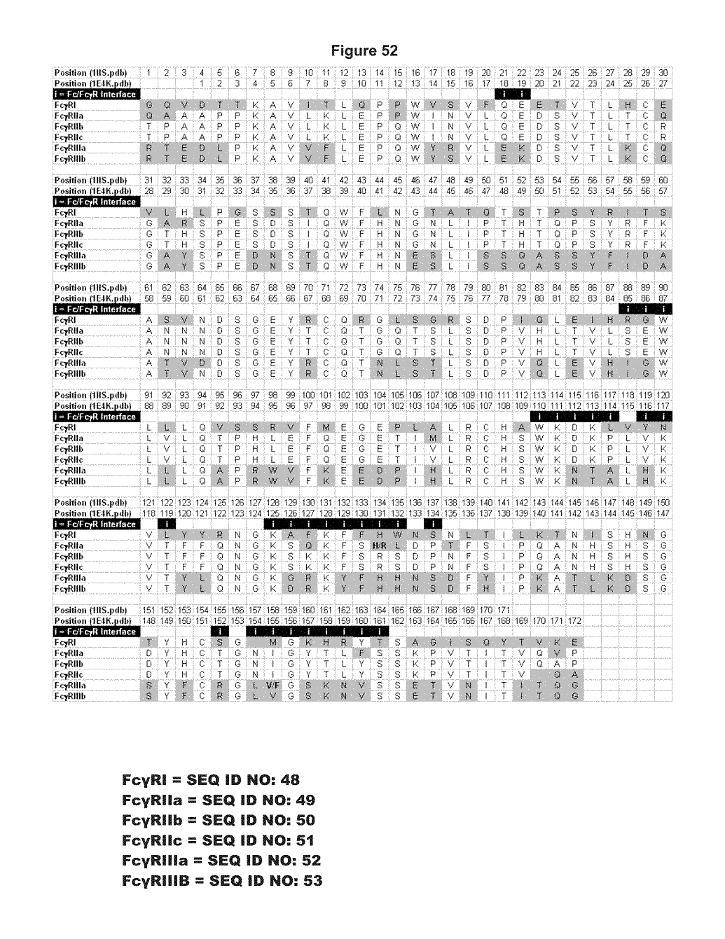

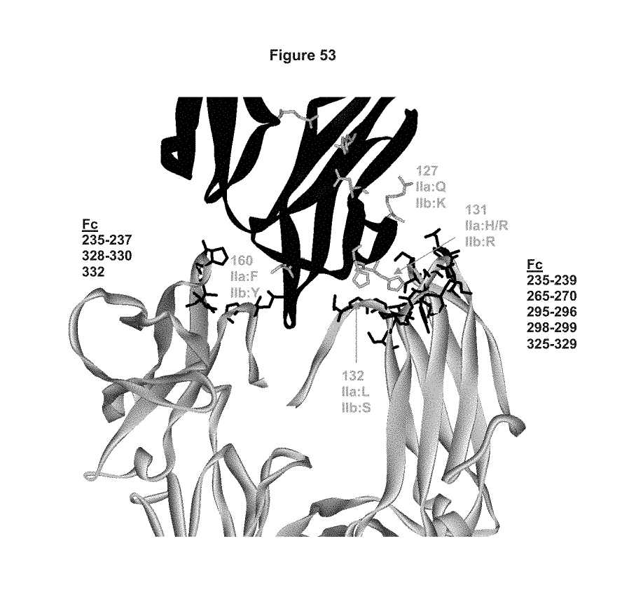

Sequence and structural analysis of the Fc/FcγR interface was carried out for the different human FcγRs. A central goal was to generate variants with selectively increased affinity for the activating receptors FcγRI, FcγRIIa, FcγRIIc, and FcγRIIIa relative to the inhibitory receptor FcγRIIb, and selectively increased affinity for FcγRIIb relative to the activating receptors. FIG. 52 shows an alignment of the sequences of the human FcγRs, highlighting the differences from FcγRIIb and positions at the Fc interface. The analysis indicates that although there is extensive homology among the human FcγRs, there are significant differences. Particularly relevant are differences at the Fc binding interface that may be capitalized on to engineer selective Fc variants.

The utility of this analysis is illustrated using the example of FcγRIIa vs. FcγRIIb. Engineering an Fc variant that selectively improves binding to FcγRIIa relative to FcγRIIb is potentially the most challenging embodiment of the present invention, due principally to the high sequence homology of these two receptors, particularly at the Fc/FcγR interface. FIG. 52 shows that there are 3 or 4 differences between FcγRIIb and FcγRIIa (depending on allotype) that distinguish binding of these receptors to the Fc region (FIG. 52). These include differences at 127 (FcγRIIa is Gln, FcγRIIb is Lys), 131 (FcγRIIa is either His or Arg depending on the allotype, FcγRIIb is an Arg), 132 (FcγRIIa is Leu, FcγRIIb is Ser), and 160 (FcγRIIa is Phe, FcγRIIb is Tyr). FcγR numbering here is according to that provided in the 1E4K pdb structure for FcγRIIIb. Mapping of these differences onto the Fc/FcγRIIIb complex (FIG. 53) reveals that Fc residues that interact with these FcγR residues occur at Fc positions 235-237, 328-330, and 332 on the A chain and at positions 235-239, 265-270, 295-296, 298-299, and 325-329 on the B chain in the 1E4K pdb structure (FcγRs bind asymmetrically to the Fc homodimer). Thus, Fc positions 235-239, 265-270, 295-296, 298-299, 325-330, and 332 are positions that may be modified to obtain Fc variants with selectively increased affinity FcγRIIa relative to FcγRIIb. A similar analysis can be carried out for selectively altering affinity to one or more of the other activating receptors relative to the inhibitory receptor, for example, for selectively improving affinity for FcγRIIIa relative to FcγRIIb, or conversely for selectively improving affinity for FcγRIIb relative to FcγRIIIa.

FcγR binding data provided in FIG. 41 of U.S. Ser. No. 11/124,620, hereby entirely incorporated by reference, indicate that indeed amino acid modification at some of these positions provide selective enhancement or reduction in FcγR affinity. For example, G236S provides a selective enhancement to FcγRII's (FcγRIIa, FcγRIIb, and FcγRIIc) relative to FcγRI and FcγRIIIa, with a somewhat greater enhancement to FcγRIIa relative to FcγRIIb and FcγRIIc. G236A, however, is highly selectively enhanced for FcγRIIa, not only with respect to FcγRI and FcγRIIIa, but also over FcγRIIb and FcγRIIc. Selective enhancements and reductions are observed for a number of Fc variants, including a number of substitutions occurring at the analyzed above, namely 235-239, 265-270, 295-296, 298-299, 325-330, and 332. Although substitutions at some of these positions have been characterized previously (U.S. Pat. No. 5,624,821; Lund et al., 1991, J Immunol 147(8):2657-2662; U.S. Pat. No. 6,737,056; Shields et al., 2001, J Biol Chem 276(9): 6591-6604), such substitutions have not been characterized with respect to their affinities for the full set of human activating and inhibitory FcγRs.

### Example 7. Screening of Fc Variants

Amino acid modifications were engineered at these positions to generate variants with selective FcγR affinity. Fc variants were engineered in the context of the anti-CD20 antibody PRO70769 (PCT/US2003/040426, hereby entirely incorporated by reference). The genes for the variable regions of PRO70769 (FIGS. 75a and 75b) were constructed using recursive PCR, and subcloned into the mammalian expression vector pcDNA3.1Zeo (Invitrogen) comprising the full length light kappa (Cκ) and heavy chain IgG1 constant regions. Amino acid substitutions were constructed in the variable region of the antibody in the pcDNA3.1Zeo vector using quick-change mutagenesis techniques (Stratagene). DNA was sequenced to confirm the fidelity of the sequences. Plasmids containing heavy chain gene (VH-CH1-CH2-CH3) (wild-type or variants) were co-transfected with plasmid containing light chain gene (VL-Cx) into 293T cells. Media were harvested 5 days after transfection, and antibodies were purified from the supernatant using protein A affinity chromatography (Pierce).

Binding affinity to human FcγRs by Fc variant anti-CD20 antibodies was measured using a competitive AlphaScreen™ assay. The AlphaScreen is a bead-based luminescent proximity assay. Laser excitation of a donor bead excites oxygen, which if sufficiently close to the acceptor bead will generate a cascade of chemiluminescent events, ultimately leading to fluorescence emission at 520-620 nm. The AlphaScreen was applied as a competition assay for screening the antibodies. Wild-type IgG1 antibody was biotinylated by standard methods for attachment to streptavidin donor beads, and tagged FcγR was bound to glutathione chelate acceptor beads. In the absence of competing Fc polypeptides, wild-type antibody and FcγR interact and produce a signal at 520-620 nm. Addition of untagged antibody competes with wild-type Fc/FcγR interaction, reducing fluorescence quantitatively to enable determination of relative binding affinities.

In order to screen for Fc/FcγR binding, the extracellular regions of human FcγRs were expressed and purified. The extracellular regions of these receptors were obtained by PCR from clones obtained from the Mammalian Gene Collection (MGC), or generated de novo using recursive PCR. To enable purification and screening, receptors were fused C-terminally with either a His tag, or with His-glutathione S-Transferase (GST). Tagged FcγRs were transfected into 293T cells, and media containing secreted receptor were harvested 3 days later and purified using Nickel chromatography. Additionally, some His-tagged FcγRs were purchased commercially from R&D Systems.

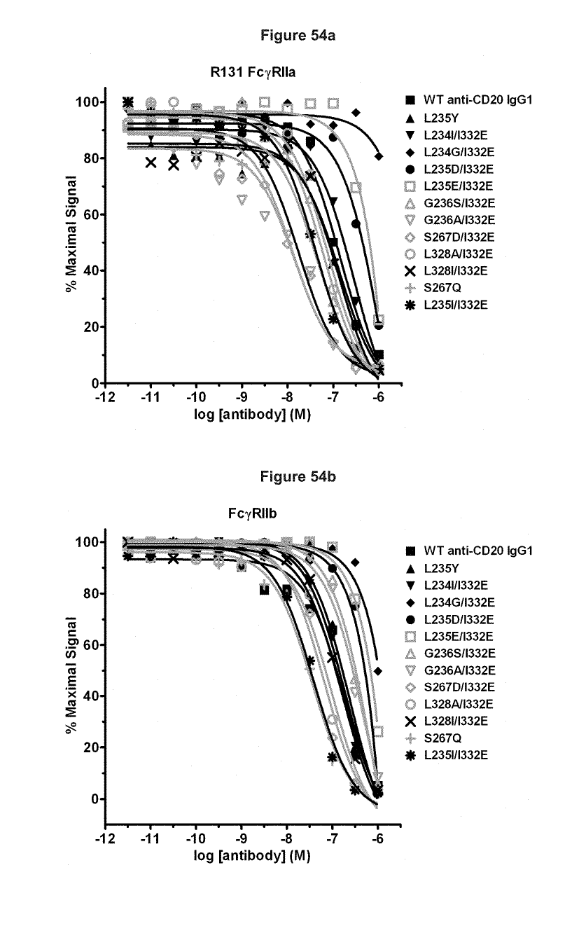

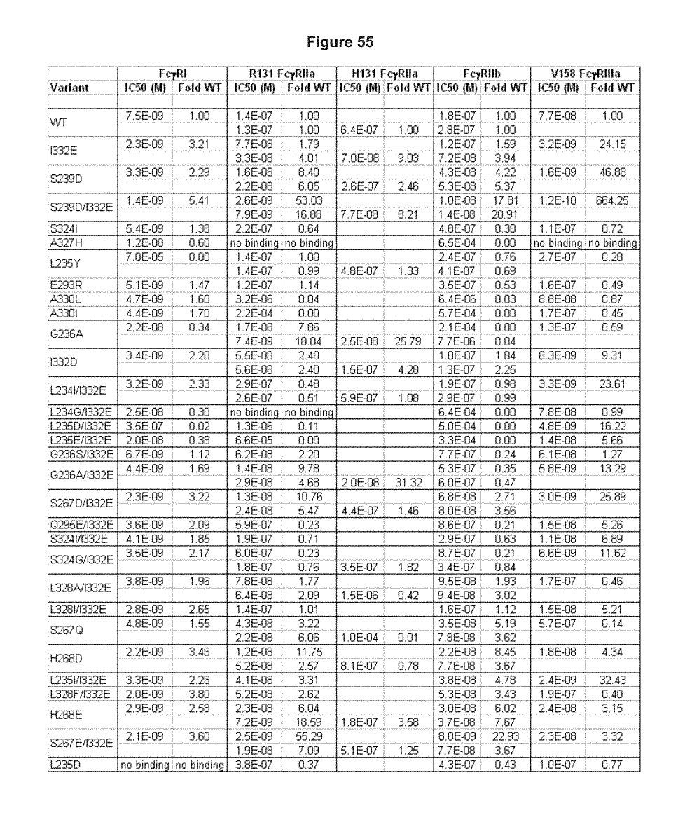

Competition AlphaScreen data were acquired for binding of the Fc variants to human FcγRI, R131 FcγRIIa, H131 FcγRIIa, FcγRIIb, and V158 FcγRIIIa. FIG. 54 shows the data for binding of select antibody variants to the human receptors R131 FcγRIIa (FIG. 54a) and FcγRIIb (FIG. 54b). The data were fit to a one site competition model using nonlinear regression, and these fits are represented by the curves in the figure. These fits provide the inhibitory concentration 50% (IC50) (i.e., the concentration required for 50% inhibition) for each antibody, thus enabling the relative binding affinities relative to WT to be determined. FIG. 55 provides the IC50's and Fold IC50's relative to WT for fits to these binding curves for all of the anti-CD20 antibody Fc variants tested. The data support the analysis above that substitution at positions within the binding region defined by 235-239, 265-270, 295-296, 298-299, 325-330, and 332 may be involved in distinguishing the different affinities of the Fc region for the different FcγRs. For example, as shown by the data, variants comprising modifications at 235, 236, 267, and 328 have varying affinity improvements and reductions relative to the parent antibody for the different FcγRs, including even the highly homologous FcγRIIa and FcγRIIb. It is notable that, with respect to engineering optimal FcγR selectivity for antibodies and Fc fusions, single variants do not necessarily provide favorable FcγR affinities. For example, although the single variant G236A provides selectively improved affinity to FcγRIIa relative to FcγRIIb, it is reduced in affinity for both the other activating receptors FcγRI and FcγRIIIa. However, combination of this substitution with other modifications that provide increased affinity to these other activating receptors, for example 1332E, results in an Fc variant with a promising FcγR affinity profile, namely increased affinity for FcγRIIa and FcγRIIIa relative to the inhibitory receptor FcγRIIb.

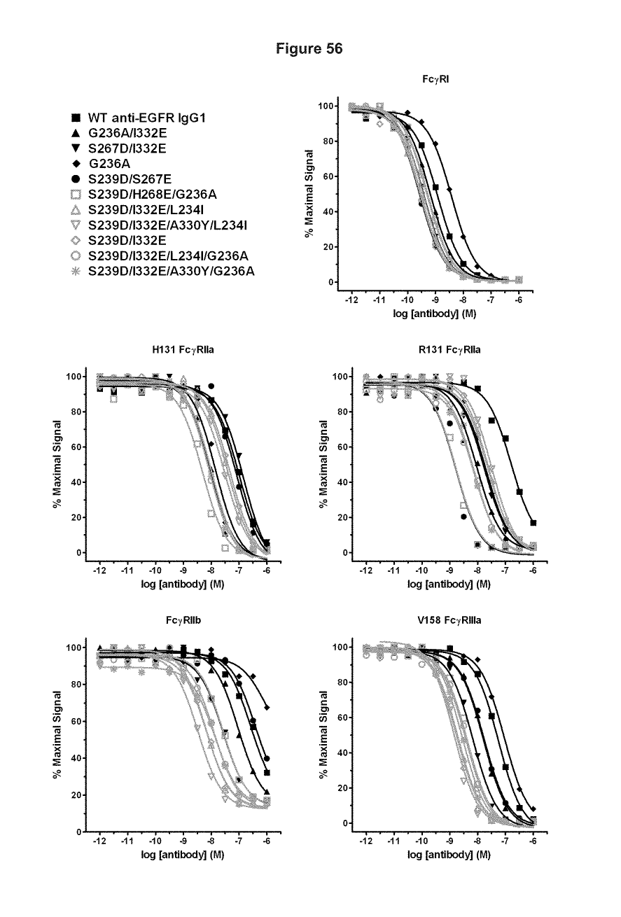

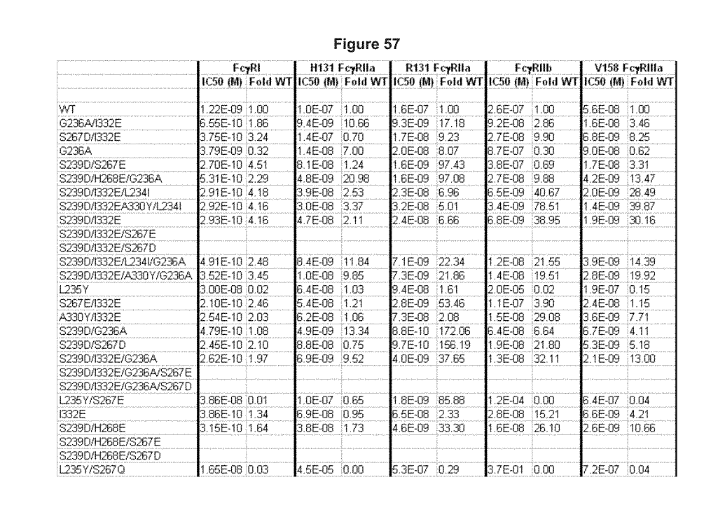

Based on these results, a number of additional Fc variants were constructed in the context of the anti-EGFR antibody H4.40/L3.32 C225 (FIGS. 75c and 75d) as disclosed in U.S. Ser. No. 60/778,226, filed Mar. 2, 2006, entitled “Optimized anti-EGFR antibodies”, herein expressly incorporated by reference). Antibody variants were constructed in the IgG1 pcDNA3.1Zeo vector, expressed in 293T cells, and purified as described above. Binding affinity to human FcγRs by Fc variant anti-EGFR antibodies was measured using a competition AlphaScreen assay as described above. FIG. 56 shows binding data for the Fc variants to human FcγRI, R131 FcγRIIa, H131 FcγRIIa, FcγRIIb, and V158 FcγRIIIa. FIG. 57 provides the IC50's and Fold IC50's relative to WT for fits to these binding curves for all of the anti-EGFR antibody Fc variants tested. The data indicate that it is possible to combine modifications at the aforementioned positions to generate variants with selectively improved affinity for one or more human activating receptors relative to the human inhibitory receptor FcγRIIb.

Based on these results, a number of additional Fc variants were constructed in the context of the anti-EpCAM antibody H3.77/L3 17-1A (FIGS. 75e and 75f) as disclosed in U.S. Ser. No. 11/484,183 and U.S. Ser. No. 11/484,198, filed in Jul. 10, 2006, herein expressly incorporated by reference). Antibody variants were constructed in the pcDNA3.1Zeo vector as described above. Antibody variants were constructed in the context of the IgG1 heavy chain and/or in the context of a novel IgG molecule referred to as IgG(hybrid) (FIG. 49g), described in U.S. Ser. No. 11/256,060, filed Oct. 21, 2005, hereby entirely incorporated by reference. Antibodies were expressed in 293T cells, and purified as described above.

Binding affinity to human FcγRs by Fc variant anti-EpCAM antibodies was measured using surface plasmon resonance (SPR), also referred to as BIAcore. SPR measurements were performed using a BIAcore 3000 instrument (BIAcore, Uppsala Sweden). Running buffer was 10 mM HEPES pH 7.4, 150 mM NaCl, 3 mM EDTA, 0.005% v/v Surfactant P20 (HBS-EP, BIAcore), and chip regeneration buffer was 10 mM glycine-HCl pH 1.5. 100 nM WT or variant anti-EpCAM antibody was bound to the protein A/G CMS chip in HBS-EP at 1 μtl/min for 5 min. 50 μtl FcγR-His analyte, in serial dilutions between 30 and 1000 nM, was injected in HBS-EP at 25 μtl/min for 2 minutes association, followed by a dissociation phase with buffer alone. Data were normalized for baseline response, obtained from a cycle with antibody and buffer alone. Response sensorgrams were fit to a 1:1 Langmuir binding model within BIAevaluation software, providing the association (ka) and dissociation (kd) rate constants, and the equilibrium dissociation constant (KD).

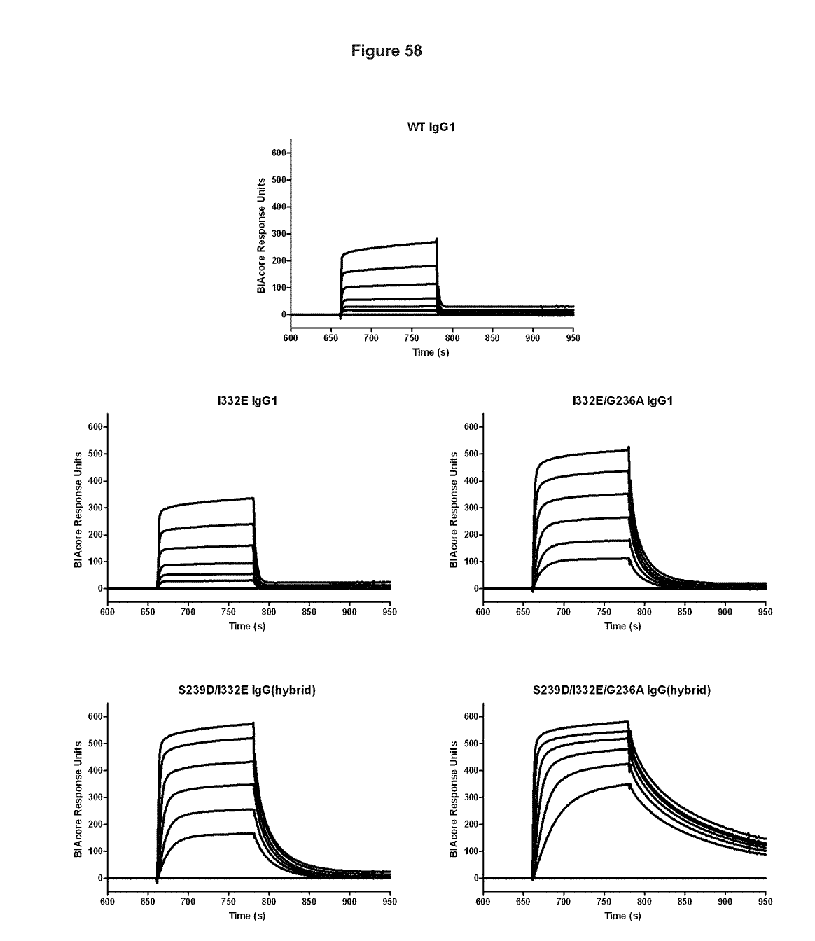

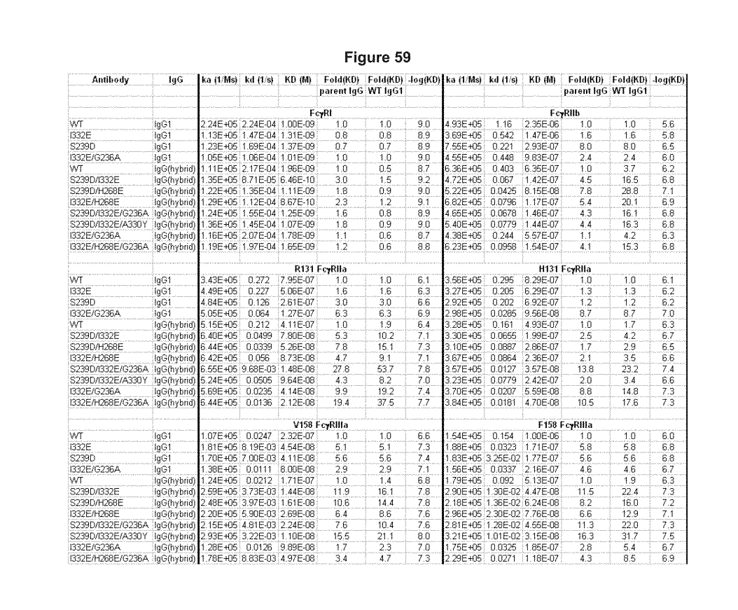

FIG. 58 shows SPR sensorgrams for binding of select anti-EpCAM Fc variants to human R131 FcγRIIa. FIG. 59 shows kinetic and equilibrium constants obtained from the fits of the SPR data for all of the receptors, well as the calculated Fold(KD) relative to WT and the negative log of the KD (−log(KD). Here Fold(KD) for a given variant to a given receptor is defined as:

Fold(KD)_{FcγR}=KD_{WT}/KDvariant Equation 1:

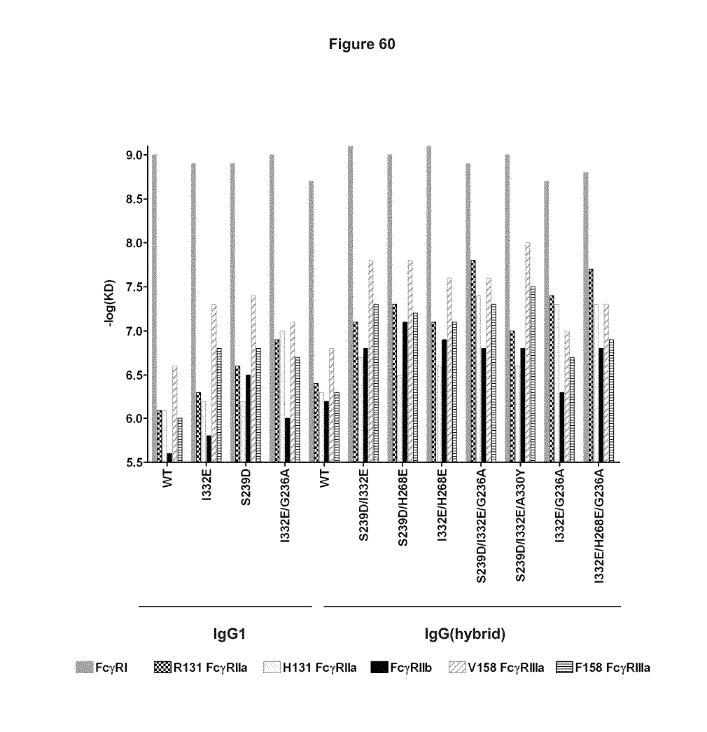

A Fold(KD) greater than 1 for a given receptor indicates that the variant improves affinity relative to the WT parent, whereas a Fold(KD) less than 1 indicates the variant reduces affinity relative to the WT parent. FIG. 60 provides a plot of the negative log of the K_{D} for binding of select anti-EpCAM Fc variants to the set of human FcγRs. Here greater −log(K_{D}) on the y-axis corresponds to tighter affinity for the receptor. In order to better view the impact of the substitutions on FcγR specificity, the activating versus inhibitory FcγR affinity differences are plotted for FcγRIIa vs. FcγRIIb and FcγRIIIa vs. FcγRIIb. Here for each variant the −log(KD) for its binding to FcγRIIb is subtracted from the −log(KD) for it binding to the activating receptor, providing a direct measure of FcγR selectivity of the variants. Notably, all variants comprising the G236A substitution, including I332E/G236A, S239D/I332E/G236A, and I332E/H268E/G236A have favorable FcγRIIa:FcγRIIb selectivity relative to, respectively, the 1332E, S239D/I332E, and I332E/H268E variants alone. Thus, the results show that suboptimal G236A substitution can be combined with other substitutions that have favorable FcγR affinities to generate Fc variants with the most optimal FcγR affinity profiles.

In order to calculate the selective enhancement in affinity for the activating receptors relative to the inhibitory receptor FcγRIIb for each variant, this analysis must be carried out with respect to the parent antibody, either WT IgG1 or WT IgG(hybrid) in this example. The selective enhancement in affinity for FcγRIIa relative to FcγRIIb provided by an Fc variant is defined as Fold(KD)_{FcγRIIa}: Fold(KD)_{FcγRIIb}, also written as Fold(KD)_{FcγRIIa}/Fold(KD)_{FcγRIIb}. This value is calculated as follows:

Fold(KD)_{FcγRIIa}: Fold(KD)_{FcγRIIb}=Fold(KD)_{FcγRIIa}/Fold(KD)_{FcγRIIb} Equation 2:

Likewise the selective enhancement in affinity for FcγRIIIa relative to FcγRIIb provided by an Fc variant is calculated as follows:

Fold(KD)_{FcγRIIa}:Fold(KD)_{FcγRIIB}=Fold(KD)_{FcγRIIIa}/Fold(KD)_{FcγRIIb} Equation 3:

FIG. 61b provides these values for both R131 and H131 isoforms of FcγRIIa (RIIa and HIIa for brevity), and for both V158 and F158 isoforms of FcγRIIIa (VIIIa and FIIIa for brevity). FIG. 61c provides a plot of these data. The results show that the Fc variants of the invention provide up to 9-fold selective enhancements in affinity for binding to the activating receptor FcγRIIa relative to the inhibitory receptor FcγRIIb, and up to 4-fold selective enhancements in affinity for binding to the activating receptor FcγRIIIa relative to the inhibitory receptor FcγRIIb.

### Example 8. Performance of Fc Variants in Cell-Based Assays

A central goal of improving the activating FcγR vs. inhibitory FcγR profile of an antibody or Fc fusion was to enhance its FcγR-mediated effector function in vitro and ultimately in vivo. To investigate the capacity of antibodies comprising the Fc variants of the present invention to carry out FcγR-mediated effector function, in vitro cell-based ADCC assays were run using human PBMCs as effector cells. ADCC was measured by the release of lactose dehydrogenase using a LDH Cytotoxicity Detection Kit (Roche Diagnostic). Human PBMCs were purified from leukopacks using a ficoll gradient, and the EpCAM+target gastric adenocarcinoma line LS180. Target cells were seeded into 96-well plates at 10,000 cells/well, and opsonized using Fc variant or WT antibodies at the indicated final concentration. Triton X100 and PBMCs alone were run as controls. Effector cells were added at 40:1 PBMC5:target cells, and the plate was incubated at 37° C. for 4 hrs. Cells were incubated with the LDH reaction mixture, and fluorescence was measured using a Fusion™ Alpha-FP (Perkin Elmer). Data were normalized to maximal (triton) and minimal (PBMCs alone) lysis, and fit to a sigmoidal dose-response model. FIG. 62 provides these data for select Fc variant antibodies. The G236A variant mediates reduced ADCC relative to WT, due likely to its reduced affinity for FcγRIIIa and/or FcγR1. ADCC in PBMCs is potentially dominated by NK cells, which express only FcγRIIIa, although in some cases they can express FcγRIIc. Thus, the reduced ADCC of the G236A single variant is consistent with its reduced affinity for this receptor. However, combination of the G236A substitution with modifications that improve affinity for these activating receptors, for example including but not limited to substitutions at 332 and 239, provide substantially improved ADCC relative to the parent WT antibody.

Monocyte-derived effector cells, including for example macrophages, express not only FcγRIIIa, but also FcγR1, FcγRIIa, and the inhibitory receptor FcγRIIb. Macrophages are phagocytes that act as scavengers to engulf dead cells, foreign substances, and other debris. Importantly, macrophages are professional antigen presenting cells (APCs), taking up pathogens and foreign structures in peripheral tissues, then migrating to secondary lymphoid organs to initiate adaptive immune responses by activating naive T-cells. Unlike NK cells, macrophages express the range of FcγRs, and thus their activation and function may be dependent on engagement of antibody immune complexes with receptors other than only FcγRIIIa.

A cell-based ADCP assay was carried out to evaluate the capacity of the Fc variants to mediate phagocytosis. Monocytes were purified from PBMCs and differentiated into macrophages in 50 ng/ml M-CSF for 5 days. Quantitated receptor expression density of FcγRI (CD64), FcγRIIa and FcγRIIb (CD32), and FcγRIIIa (CD16) on these cells was determined with standard flow cytometry methods using PE (orange)—labeled anti-FcγRs and biotinylated PE-Cy5-labeled antibodies against macrophage markers CD11b and CD14. PE-conjugated anti-CD64 (Clone 10.1) was purchased from eBioscience, PE-conjugated anti-CD32 (Clone 3D3) and PE-conjugated anti-CD16 (Clone 3G8) were purchased from BD Bioscience. Biotinylated anti-CD14 (TUK4) was purchased from Invitrogen, and biotinylated anti-CD11b (Clone ICRF44) was purchased from BD Bioscience. Secondary detection was performed with streptavidin PE-Cy5 obtained from Biolegend. Cytometry was carried out on a Guava Personal Cell Analysis-96 (PCA-96) System (Guava Technologies). FIG. 63a shows that the monocyte-derived macrophages (MDM) express high levels of FcγRII (99%) and FcγRIII (81%), and moderate (45%) levels of FcγRI. The inability to distinguish between FcγRIIa and FcγRIIb is due to the unavailability of commercial antibodies that selectively bind these two receptors.

For ADCP assays with MDM as effector cells, target EpCAM+LS180 cells were labeled with PKH26 and plated in a 96-well round bottom plate at 25 000 cells/well. Antibodies (WT and Fc variants) were added to wells at indicated concentrations, and antibody opsinized cells were incubated for approximately 30 minutes prior to the addition of effector cells. Monocyte derived macrophages (MDM) were added to each well at approximately 4:1 effector to target ratio, and the cells were incubated overnight. Cells were washed and treated with HyQtase. MDM were stained with biotinylated CD11b and CD14, followed by a secondary stain with Streptavidin PE-Cy5. Cells were fixed in 1% paraformaldehyde and read on the Guava flow cytometer.

FIG. 63b shows the results of an ADCP assay of select anti-EpCAM Fc variants in the presence of macrophages. FIG. 63c show a repeat experiment with some of these variants. The data show that the improved FcγRII:FcγRIIb profile of the I332E/G236A variant relative to the I332E single variant provides enhanced phagocytosis. Interestingly, G236A does not improve phagocytosis of the S239D/I332E variant. The reason(s) for this result are not clear, but may be due in part to the lower FcγRI binding affinity of S239D/I332E/G236A relative to S239D/I332E, whereas I332E/G236A does not have compromised FcγRI affinity relative to I332E alone. Alternatively, it may be that the inhibitory receptor FcγRIIb, the affinity for which is greater in the S239D/I332E and S239D/I332E/G236A variants relative to the I332E and I332E/G236A variants, establishes an absolute threshold of activation/repression. That is, regardless of how much affinity to FcγRIIa is improved, at a certain level of FcγRIIb engagement cellular activation and effector function is inhibited.

Dendritic cells (DCs) are professional antigen presenting cells (APCs) that take up pathogens/foreign structures in peripheral tissues, then migrate to secondary lymphoid organs where they initiate adaptive immune responses by activating naive T-cells. Immature DCs endocytose either free or complexed antigens in the periphery, and this stimulus induces their maturation and migration to secondary lymphoid organs. Mature DCs expressing costimulatory molecules and produce various cytokines, including for example TNFα, to efficiently activate antigen-specific naive T-cells. DC-derived cytokines play a crucial role in shaping the adaptive response via determining polarization of T-cells towards either the Th1 or the Th2 phenotype (Bajtay et al., 2006, Immunol Letters 104: 46-52). Human DCs can express the various FcγRs depending on their source and activation state (Bajtay et al., 2006, Immunol Letters 104: 46-52). In contrast to circulating monocytic precursors to DCs, which can express the range of FcγRs, immature monocyte-derived DCs express primarily FcγRIIa and FcγRIIb. Recent data suggest that the relative engagement of FcγRIIa and FcγRIIb by immune complexes establishes a threshold of DC activation, mediating opposing effects on DC maturation and function (Boruchov et al., 2005, J Clin Invest 115(10):2914-23).

To evaluate the effect of the different FcγR affinity profiles on DC maturation, a cell-based assay was carried out using TNFα release to monitor DC activation. Dendritic cells (DCs) were generated from CD14+ sorted cells that were cultured in the presense of GM-CSF (1000 Units/ml or 100 ng/ml) and IL4 (500 Units/ml or 100 ng/ml) for six days. FcγRIIa and FcγRIIb (CD32), and FcγRIIIa (CD16) expression on these cells was determined with standard flow cytometry methods using PE-labeled anti-FcγRs. PE-conjugated anti-CD64 (Clone 10.1) was purchased from eBioscience, PE-conjugated anti-CD32 (Clone 3D3) and PE-conjugated anti-CD16 (Clone 3G8) were purchased from BD Bioscience. Cytometry was carried out on the Guava. FIG. 64a shows that the DCs used express high levels of FcγRII (94.7%), low to moderate levels of FcγRIII (37.2%), and low to no FcγRI (7.3%).

For the DC activation assay, DCs were cultured in the presense of various concentrations of antibody and EpCAM+LS180 cells overnight. Supernatants were harvested and tested for TNFα by ELISA. FIG. 64b shows the dose response curves for TNFα release by DCs in the presence of WT and Fc variant antibodies. The data show that DC activation is correlated roughly with the FcγRIIa:FcγRIIb affinity ratio (FIG. 61), consistent with the literature and the dominant expression of FcγRII receptors on the DCs used in the present assay. I332E and S239D/I332E mediate DC activation comparable with or lower than WT, in agreement with their FcγRIIa:FcγRIIb affinity profile. However addition of a substitution that selectively improves the FcγR affinity for FcγRIIa relative to FcγRIIb, in this case G236A, dramatically improves DC activation—I332E/G236A and S239D/I332E/G236A show enhanced DC activation relative to WT, I332E, and S239D/I332E. Together the macrophage phagocytosis and DC activation data are the first examples of the use of antibody Fc variants with improved FcRIIa:FcγRIIb affinity profiles to enhance the function of antigen presenting cells. Along with the ADCC data (FIG. 62), the cell-based results indicate that the most optimal engineered FcγR profile is selectively improved affinity for both FcγRIIa and FcγRIIIa relative to the inhibitory receptor FcγRIIb, for example as provided by the combination of S239D, I332E, and G236A substitutions.

### Example 9. Preferred Fc Variants of the Invention

Taken together, the data provided in the present invention indicate that combinations of amino acid modifications at positions 235, 236, 237, 238, 239, 265, 266, 267, 268, 269, 270, 295, 296, 298, 299, 325, 326, 327, 328, 329, 330, and 332 provide promising candidates for selectively modifying the FcγR binding properties, the effector function, and potentially the clinical properties of Fc polypeptides, including antibodies and Fc fusions. In particular, Fc variants that selectively improve binding to one or more human activating receptors relative to FcγRIIb, or selectively improve binding to FcγRIIb relative to one or more activating receptors, may comprise a substitution, as described herein, selected from the group consisting of 234G, 234I, 235D, 235E, 235I, 235Y, 236A, 236S, 239D, 267D, 267E, 267Q, 268D, 268E, 293R, 295E, 324G, 324I, 327H, 328A, 328F, 328I, 330I, 330L, 330Y, 332D, and 332E. Additional substitutions that may also be combined include other substitutions that modulate FcγR affinity and complement activity, including but not limited to 298A, 298T, 326A, 326D, 326E, 326W, 326Y, 333A, 333S, 334L, and 334A (U.S. Pat. No. 6,737,056; Shields et al, Journal of Biological Chemistry, 2001, 276(9):6591-6604; U.S. Pat. No. 6,528,624; Idusogie et al., 2001, J. Immunology 166:2571-2572). Preferred variants that may be particularly useful to combine with variants of the present invention include those that comprise the substitutions 298A, 326A, 333A, and 334A. AlphaScreen data measuring the binding of Fc variants comprising these substitutions to the human activating receptors V158 and F158 FcγRIIIa and the inhibitory receptor FcγRIIb are shown in FIG. 65a-65c. Additional substitutions that may be combined with the FcγR selective variants of the present invention 247L, 255L, 270E, 392T, 396L, and 421K (U.S. Ser. No. 10/754,922; U.S. Ser. No. 10/902,588), and 280H, 280Q, and 280Y (U.S. Ser. No. 10/370,749), all of which are herein expressly incorporated by reference.

In particularly preferred embodiments of the invention, Fc variants of the present invention may be combined with Fc variants that alter FcRn binding. In particular, variants that increase Fc binding to FcRn include but are not limited to: 250E, 250Q, 428L, 428F, 250Q/428L (Hinton et al., 2004, J. Biol. Chem. 279(8): 6213-6216, Hinton et al., 2006, Journal of Immunology 176:346-356, U.S. Ser. No. 11/102,621, PCT/US2003/033037, PCT/US2004/011213, U.S. Ser. No. 10/822,300, U.S. Ser. No. 10/687,118, PCT/US2004/034440, U.S. Ser. No. 10/966,673 all entirely incorporated by reference), 256A, 272A, 286A, 305A, 307A, 311A, 312A, 376A, 378Q, 380A, 382A, 434A (Shields et al., Journal of Biological Chemistry, 2001, 276(9):6591-6604, U.S. Ser. No. 10/982,470, U.S. Pat. No. 6,737,056, U.S. Ser. No. 11/429,793, U.S. Ser. No. 11/429,786, PCT/US2005/029511, U.S. Ser. No. 11/208,422, all entirely incorporated by reference), 252F, 252T, 252Y, 252W, 254T, 256S, 256R, 256Q, 256E, 256D, 256T, 309P, 311S, 433R, 433S, 4331, 433P, 433Q, 434H, 434F, 434Y, 252Y/254T/256E, 433K/434F/436H, 308T/309P/311S (Dall Acqua et al., 2002, Journal of Immunology, 169:5171-5180, U.S. Pat. No. 7,083,784, PCT/US97/03321, U.S. Pat. No. 6,821,505, PCT/US01/48432, U.S. Ser. No. 11/397,328, all entirely incorporated by reference), 257C, 257M, 257L, 257N, 257Y, 279E, 279Q, 279Y, insertion of Ser after 281, 283F, 284E, 306Y, 307V, 308F, 308Y 311V, 385H, 385N, (PCT/US2005/041220, U.S. Ser. No. 11/274,065, U.S. Ser. No. 11/436,266 all entirely incorporated by reference) 204D, 284E, 285E, 286D, and 290E (PCT/US2004/037929 entirely incorporated by reference).

Preferred combinations of positions and modifications are summarized in FIG. 66.

This list of preferred Fc variants is not meant to constrain the present invention. Indeed all combinations of the any of the Fc variants provided are embodiments of the present invention. Furthermore, combinations of any of the Fc variants of the present invention with other discovered or undiscovered Fc variants may also provide favorable properties, and these combinations are also contemplated as embodiments of the present invention. Further, substitutions at all positions disclosed herein are contemplated.

### Example 10. Fc Variants Comprising Amino Acid Modifications and Engineered Glycoforms that Provide Selective FcγR Affinity

An alternative method to amino acid modification for modulating FcγR affinity of an Fc polypeptide is glycoform engineering. As discussed, antibodies are post-translationally modified at position 297 of the Fc region with a complex carbohydrate moiety. It is well known in the art that this glycosylation plays a role in the functional fidelity of the Fc region with respect to binding Fc ligands, particularly FcγRs and complement. It is also well established in the art that Fc polypeptide compositions that comprise a mature core carbohydrate structure which lacks fucose have improved FcγR affinity relative to compositions that comprise carbohydrate that is fucosylated (Umaña et al., 1999, Nat Biotechnol 17:176-180; Davies et al., 2001, Biotechnol Bioeng 74:288-294; Shields et al., 2002, J Biol Chem 277:26733-26740; Shinkawa et al., 2003, J Biol Chem 278:3466-3473); (U.S. Pat. No. 6,602,684; U.S. Ser. No. 10/277,370; U.S. Ser. No. 10/113,929; PCT WO 00/61739A1; PCT WO 01/29246A1; PCT WO 02/31140A1; PCT WO 02/30954A1). However, previous studies have shown that although reduction of fucose content improves the affinity of an IgG for human FcγRIIIa, it has no effect on binding to human FcγRI, either isoform (R131 or H131) of human FcγRIIa, or human FcγRIIb (U.S. Ser. No. 10/277,370; Shields et al., 2002, J Biol Chem 277(90):26733-26740). Recent experiments have determined that the high affinity between glycoengineered antibodies and FcγRIII is mediated by productive interactions formed between the receptor carbohydrate attached at Asn162 and regions of the Fc that are only accessible when it is nonfucosylated. Because FcγRIIIa and FcγRIIIb are the only human Fc receptors glycosylated at this position, the proposed interactions explain the observed selective affinity increase of glycoengineered antibodies for only these receptors (Ferrara et al., 2006, J Biol Chem 281(8):5032-5036).

The data provided in Example 11 suggest that combination of glycoform engineering with FcγR selective amino acid modifications may provide Fc variants with selectively improved affinity for one or more activating receptors relative to the inhibitory receptor FcγRIIb.

In order to explore whether amino acid modification would enable such selective FcγR binding, we evaluated preferred amino acid substitutions in the context of antibodies with reduced fucose content. The Lec13 cell line (Ripka et al., Arch. Biochem. Biophys. 49:533-545 (1986)) was utilized to express human antibodies with reduced fucose content. Lec13 refers to the lectin-resistant Chinese Hamster Ovary (CHO) mutant cell line which displays a defective fucose metabolism and therefore has a diminished ability to add fucose to complex carbohydrates. That cell line is described in Ripka & Stanley, 1986, Somatic Cell & Molec. Gen. 12(1):51-62; and Ripka et al., 1986, Arch. Biochem. Biophys. 249(2):533-545. Lec13 cells are believed lack the transcript for GDP-D-mannose-4,6-dehydratase, a key enzyme for fucose metabolism. Ohyama et al., 1988, J. Biol. Chem. 273(23):14582-14587. GDP-D-mannose-4,6-dehydratase generates GDP-mannose-4-keto-6-D-deoxymannose from GDP-mannose, which is then converted by the FX protein to GDP-L-fucose. Expression of fucosylated oligosaccharides is dependent on the GDP-L-fucose donor substrates and fucosyltransferase(s). The Lec13 CHO cell line is deficient in its ability to add fucose, but provides IgG with oligosaccharide which is otherwise similar to that found in normal CHO cell lines and from human serum (Jefferis, R. et al., 1990, Biochem. J. 268, 529-537; Raju, S. et al., 2000, Glycobiology 10, 477-486; Routier, F. H., et al., 1997, Glycoconj. J. 14, 201-207). Normal CHO and HEK293 cells add fucose to IgG oligosaccharide to a high degree, typically from 80-98%, and IgGs from sera are also highly fucosylated (Jefferis, R. et al., 1990, Biochem. J. 268, 529-537; Raju, S. et al., 2000, Glycobiology 10, 477-486; Routier, F. H., et al., 1997, Glycoconj. J. 14, 201-207; Shields et al., 2002, J Biol Chem 277(90):26733-26740). It is well established that antibodies expressed in transfected Lec13 cells consistently produce about 10% fucosylated carbohydrate (Shields et al., 2002, J Biol Chem 277(90):26733-26740).

WT, G236A, and S239D/I332E variant anti-EpCAM antibodies were each transiently expressed in 293T and Lec13 cells and purified as described above. Binding affinity to human FcγRI, H131 FcγRIIa, R131 FcγRIIa, FcγRIIb, and V158 FcγRIIIa by Fc variant anti-EpCAM antibodies was measured using the SPR experiment described above. FIG. 67 provides the equilibrium constants obtained from the fits of the SPR data for all of the receptors, as well as the calculated fold KD relative to WT and the negative log of the KD (−log(KD). FIG. 68 provides a plot of the negative log of the KD for binding of the antibodies to the set of human FcγRs. The data confirm that reduced fucosylation provides an increase in affinity only for FcγRIIIa, and does not alter affinity for any of the other FcγRs. However combination of glycoengineering with a substitution that selectively improves the FcγR affinity for FcγRIIa relative to FcγRIIb, in this case G236A, provides the optimal FcγR affinity profile of selectively improved affinity for FcγRIIa and FcγRIIIa relative to the inhibitory receptor FcγRIIb. Given the macrophage phagocytosis and DC activation data provided above, this novel combination of glycoengineering and amino acid substitutions with selective FcγR affinity profiles has the potential for producing more efficacious therapeutic antibodies than glycoengineering alone.

The use of the Lec13 cell line is not meant to limit the present invention to that particular mode of reducing fucose content. A variety of other methods are known in the art for controlling the level of fucosylated and/or bisecting oligosaccharides that are covalently attached to the Fc region, including but not limited to expression in various organisms or cell lines, engineered or otherwise (for example, Lec13 CHO cells or rat hybridoma YB2/0 cells), regulation of enzymes involved in the glycosylation pathway (for example FUT8 [a1,6-fucosyltranserase] and/or β1-4- N-acetylglucosaminyltransferase III [GnTIII]), and modification of modifying carbohydrate(s) after the IgG has been expressed (Umaña et al., 1999, Nat Biotechnol 17:176-180; Davies et al., 2001, Biotechnol Bioeng 74:288-294; Shields et al., 2002, J Biol Chem 277:26733-26740; Shinkawa et al., 2003, J Biol Chem 278:3466-3473); (U.S. Pat. No. 6,602,684; U.S. Ser. No. 10/277,370; U.S. Ser. No. 10/113,929; PCT WO 00/61739A1; PCT WO 01/29246A1; PCT WO 02/31140A1; PCT WO 02/30954A1).

### Example 11. Additional Fc Variant Combinations

Substitutions were engineered in the context of the S239D, 1332E, and S239D/I332E variants to explore additional Fc variants with optimized FcγR binding properties. Variants were constructed with the variable region of the anti-CD30 antibody H3.69 V2/L3.71 AC10 (FIGS. 75g and 75h) as disclosed in U.S. Ser. No. 60/776,598, filed Feb. 24, 2006, entitled “Optimized anti-CD30 antibodies”, herein expressly incorporated by reference). Antibody variants were constructed in the IgG(hybrid) pcDNA3.1Zeo vector, expressed in 293T cells, and purified as described above. Binding affinity to human FcγRs by Fc variant anti-CD30 antibodies was measured using the competition AlphaScreen assay as described above. FIG. 69 shows binding data for select Fc variants to human V158 FcγRIIIa. FIG. 70 provides the Fold IC50's relative to WT for fits to these binding curves for all of the anti-CD30 antibody Fc variants tested.

### Example 12. Mouse IgG Fc Variants with Optimized Affinity for Mouse FcγRs

The biological properties of antibodies and Fc fusions have been tested in in vivo models in order to measure a drug's efficacy for treatment against a disease or disease model, or to measure a drug's pharmacokinetics, toxicity, and other properties. A common organism used for such studies is the mouse, including but not limited to nude mice, SCID mice, xenograft mice, and transgenic mice (including knockins and knockouts). Interpretation of the results from such studies is a challenge because mouse FcγRs different substantially from human FcγRs in their homology, their expression pattern on effector cells, and their signaling biology. FIG. 23 highlights some of these key differences. FIG. 71a shows the putative expression patterns of different FcγRs on various effector cell types, and FIG. 71b shows the % identity between the human and mouse FcγR extracellular domains. Of particular importance is the existence of FcγRIV, discovered originally as CD16-2 (Mechetina et al., 2002, Immunogenetics 54:463-468) and renamed FcγRIV (Nimmerjahn & Ravetch, 2005, Science 310:1510-1512). FcγRIV is thought to be the true ortholog of human FcγRIIIa, and the two receptors are 64% identical (FIG. 23b). However whereas human FcγRIIIa is expressed on NK cells, mouse FcγRIV is not. The receptor that is expressed on mouse NK cells is FcγRIII, which shows substantially lower homology to human FcγRIIIa (49%). Interestingly, mouse FcγRIII is 93% homologous to the mouse inhibitory receptor FcγRIIb, a pair that is potentially analogous to human FcγRIIa and FcγRIIb (93% identical). However the expression pattern of mouse FcγRIII differs from that of human FcγRIIa.

These differences highlight the difficulties in interpreting results from in vivo experiments in mice using human antibodies when Fc receptor biology may affect outcome. The most optimal human antibody in humans with respect to FcγR-mediated effector function, widely viewed to be IgG1, likely does not have the optimal FcγR affinity profile for the murine receptors. Accordingly, Fc variant antibodies having optimized affinity for human Fc receptors may not provide optimal enhancements in mice, and thus may provide misleading results. The most optimal mouse FcγR affinity profile is likely provided by the most naturally optimal mouse IgG or IgGs, for example mouse IgG2a and/or IgG2b. Accordingly, engineering of mouse IgGs for optimized affinity for mouse FcγRs may provide the most informative results in in vivo experiments. In this way Fc-optimized mouse IgGs may find use as surrogate Fc-optimized antibodies in preclinical mouse models. The present invention provides mouse IgG antibodies optimized for binding to mouse FcγRs.

Fc substitutions were constructed in the context of mouse IgG1, mouse IgG2a, mouse IgG2b, and human IgG1 (FIG. 29). DNA encoding murine IgGs were obtained as IMAGE clones from the American Type Culture Collection (ATCC). Antibodies were constructed with the variable region of the anti-EGFR antibody H4.40/L3.32 C225 (FIGS. 27c and 27d) as disclosed in U.S. Ser. No. 60/778,226, filed Mar. 2, 2006, entitled “Optimized anti-EGFR antibodies”, herein expressly incorporated by reference). Antibody variants were constructed in the pcDNA3.1Zeo vector, expressed in 293T cells, and purified as described above. FIG. 24 lists the mouse and human IgG variants that were engineered.

Binding affinities to the murine activating receptors FcγRI, FcγRIII, and FcγRIV, and the murine inhibitory receptor FcγRIIb were measured using the SPR experiment described above. His-tagged murine FcγRs were purchased commercially from R&D Systems. FIG. 25 shows equilibrium constants obtained from the fits of the SPR data for the set of murine FcγRs. Also presented is the calculated fold K_{D} relative to WT murine IgG2a, potentially the most potent natural murine IgG antibody with respect to FcγR-mediated effector function (Hamaguchi et al., 2005, J Immunol 174: 4389-4399). FIG. 26 shows a plot of the negative log of the K_{D} for binding of human and mouse anti-EGFR Fc variant antibodies to mouse Fc receptors FcγRI, FcγRIIb, FcγRIII, and FcγRIV. The variants provide remarkable enhancements in binding to the murine activating receptors, particularly FcγRIV, currently thought to be one of the most relevant receptors for mediating antibody-dependent effector functions in murine xencograft models (Nimmerjahn & Ravetch, 2005, Science 310:1510-1512). The results indicate that the FcγR-binding properties of the murine IgGs can be improved using the Fc variants of the present invention, and thus may provide utility for preclinical testing of antibodies and Fc fusions that comprise Fc variants with optimized Fc receptor binding properties.

### Example 13. Fc Variants with Enhanced FcγR-Mediated Effector Function

Using the methods described in U.S. Ser. No. 10/672,280, U.S. Ser. No. 10/822,231, U.S. Ser. No. 11/124,620, and U.S. Ser. No. 11/256,060, all hereby entirely incorporated by reference, additional Fc variants were designed for enhanced binding to Fc ligands and optimized effector function, and for reduced or ablated FcγR binding and effector function. The variants were constructed in the context of the anti-CD20 antibody PRO70769 (PCT/US2003/040426, hereby entirely incorporated by reference), which is known to mediate measurable CDC and ADCC in cell-based assays. Previously characterized variants were also constructed in PRO70769, in order to further characterize their properties and provide comparators for the current set of new variants. FIG. 77 provides a list of these Fc variants. Notably, this variant set comprises a number of insertions. For example, “Insert L>235-236/I332E” refers to a double mutant comprising the substitution I332E and an insertion of leucine between residues 235 and 236.

The genes for the variable regions of PRO70769 (FIGS. 19a and 19b) were constructed using recursive PCR, and subcloned into the mammalian expression vector pcDNA3.1Zeo (Invitrogen) comprising the full length light kappa (Cκ) and heavy chain IgG1 constant regions. Variants were constructed in the variable region of the antibody in the pcDNA3.1Zeo vector using quick-change mutagenesis techniques (Stratagene), expressed in 293T cells. DNA was sequenced to confirm the fidelity of the sequences. Plasmids containing heavy chain gene (VH-CH1-CH2-CH3) (wild-type or variants) were co-transfected with plasmid containing light chain gene (VL-Cκ) into 293T cells. Media were harvested 5 days after transfection, and antibodies were purified from the supernatant using protein A affinity chromatography (Pierce). Select Fc variants were also expressed in the context of alemtuzumab.

Binding affinity to human FcγRs by IgG antibodies was measured using a competitive AlphaScreen™ assay. The AlphaScreen is a bead-based luminescent proximity assay. Laser excitation of a donor bead excites oxygen, which if sufficiently close to the acceptor bead will generate a cascade of chemiluminescent events, ultimately leading to fluorescence emission at 520-620 nm. The AlphaScreen was applied as a competition assay for screening the antibodies. Wild-type IgG1 antibody was biotinylated by standard methods for attachment to streptavidin donor beads, and tagged FcγR was bound to glutathione chelate acceptor beads. In the absence of competing Fc polypeptides, wild-type antibody and FcγR interact and produce a signal at 520-620 nm. Addition of untagged antibody competes with wild-type Fc/FcγR interaction, reducing fluorescence quantitatively to enable determination of relative binding affinities.

FIG. 78 provides competitive AlphaScreen data for binding of select PRO70769 Fc variants to the human activating receptors V158 FcγRIIIa (FIG. 78a) and F158 FcγRIIIa (FIG. 78b). The data were fit to a one site competition model using nonlinear regression, and these fits are represented by the curves in the figure. These fits provide the inhibitory concentration 50% (IC50) (i.e., the concentration required for 50% inhibition) for each antibody, thus enabling the relative binding affinities relative to WT to be determined. FIG. 77 provides the IC50's and Fold IC50's relative to WT for fits to these binding curves.

Select Fc variants were reexpressed and reetested using the competition AlphaScreen assay for binding to human V158 FcγRIIIa and F158 FcγRIIIa (FIG. 79). FIG. 79a shows the binding data for these variants, and FIG. 79b provides the IC50's and Fold IC50's relative to WT for fits to these binding curves.

Based on these data, a number of additional Fc variants were constructed in the context of PRO70769 IgG1. Additionally, some Fc variants were constructed in the context of a novel IgG molecule IgG(1/2) ELLGG described in U.S. Ser. No. 11/256,060, filed Oct. 21, 2005, hereby entirely incorporated by reference. These variants were constructed as described above, and expressed and purified along with a number of previously characterized Fc variants. These variants are listed in FIG. 80a. Binding of the variant to the human activating receptors V158 FcγRIIIa and F158 FcγRIIIa, and the inhibitory receptor FcγRIIb was measured using the competition AlphaScreen assay. FIG. 80b shows data for binding of select variants to these receptors, and FIG. 80a provides the IC50's and Folds relative to WT PRO70769 IgG1 for all of this set of Fc variants.

Because of the high avidity nature of the assay, the AlphaScreen provides only relative affinities. True binding constants were obtained using a competition SPR experiment (Nieba et al., 1996, Anal Biochem 234:155-65, hereby entirely incorporated by reference) in which unbound antibody in an antibody/FcγR equilibrium was captured to an FcγRIIIa surface. This experiment was carried out with the I332E and S239D/I332E variants in the context of trastuzumab IgG1, constructed and characterized previously (U.S. Ser. No. 10/672,280, U.S. Ser. No. 10/822,231, and U.S. Ser. No. 11/124,620, all hereby entirely incorporated by reference). WT and variant trastuzumab antibodies were expressed and purified as described above. For this experiment, data were acquired on a BIAcore 3000 instrument (BIAcore). V158 FcγRIIIa-His-GST was captured using immobilized anti-GST antibody, blocked with recombinant GST, and binding to antibody/receptor competition analyte was measured. Anti-GST antibody was covalently coupled to a CMS sensor using the BIAcore GST Capture Kit. Flow cell 1 of every sensor chip was coupled with ethanolamine as a control of unspecific binding and to subtract bulk refractive index changes online. Running buffer was HBS-EP (0.01 M HEPES pH 7.4, 0.15 M NaCl, 3 mM EDTA, 0.005% v/v Surfactant P20, BIAcore), and chip regeneration buffer was Glycine 1.5 (10 mM glycine-HCl, pH 1.5, BIAcore). 1 μM V158 FcγRIIIa-His-GST was bound to the anti-GST CMS chip in HBS-EP at 1 μl/min for 5 minutes. The surface was blocked with 5 μM recombinant GST (Sigma) injected at 1 μl/minute for 2 minutes. 100 nM wild-type or variant trastuzumab antibody was combined with V158 FcγRIIIa-His-GST in serial dilutions between 4 and 1000 nM and incubated for at least two hours at room temperature. The competition mixture was injected over the V158 FcγRIIIa-His-GST/recombinant GST surface for 30 seconds association in HBS-EP at 50 μl/minute. A cycle with antibody but no competing receptor provided a baseline response.

An earlier described “competition BIAcore” method used fitted kinetic curves to derive on-rates (Nieba et al., 1996, Anal Biochem 234:155-65, hereby entirely incorporated by reference). We found this method to be less reliable since the on-rates derived from the kinetic curves showed no linear correlation to the antibody concentration applied. The analysis used in the present study is based on the proportionality of the initial rate R to the free antibody concentration (Holwill et al., 1996, Process Control and Quality 8:133-145; Edwards & Leatherbarrow, 1997, Anal Biochem 246:1-6, all hereby entirely incorporated by reference). Response units data were exported using BIAevaluation software (BIAcore) and analyzed using Microsoft Excel with Xlfit version 3.0.5 (IDBS). Initial rate (of signal increase) values were determined from the raw data of each sensorgram using the Excel formula for slope. The equilibrium dissociation binding constant (K_{D}) was determined by plotting the log of FcγRIIIa concentration against the initial rate obtained at each concentration. GraphPad Prism (GraphPad Software) was used to fit the data to the following formula:

$$R = \frac{{R}_{0}}{2 [ {A}_{0} ]} ( [ {A}_{0} ] - {10}^{2} - {K}_{D} ) + \sqrt{( {K}_{D}^{2} + 2 ( {10}^{2} ) ( {K}_{D} ) + {( {10}^{2} )}^{2} + 2 [ {A}_{0} ] {K}_{D} - 2 [ {A}_{0} ] {10}^{2} + {[ {A}_{0} ]}^{2} )}$$

with:
[A₀]=Antibody concentration
R₀=Initial rate at antibody concentration A₀, with no competing receptor present
X=log[L₀], where [L₀]=input receptor concentration
K_{D}=Equilibrium dissociation constant
R₀ reflects the rate of binding between antibody and immobilized receptor (in the absence of competing receptor), and because of their different receptor affinities was calculated separately for WT, I332E, and S239D/I332E antibodies.
The formula for the initial rate R is derived from the definition of K_{D} for a single binding site:

$$\frac{[ {A}_{0} ] [ {L}_{0} ]}{[ {A}_{0} {L}_{0} ]} = {K}_{D}$$

and the conservation of mass

[L₀]=[L]+[A₀L₀]

with:
[L]=concentration of free receptor

Initial binding rates were determined from sensorgram raw data (FIG. 81a), and K_{D}'s were calculated by plotting the log of receptor concentration against the initial rate obtained at each concentration (FIG. 81b, 81c) (Edwards & Leatherbarrow, 1997, Anal Biochem 246:1-6, hereby entirely incorporated by reference). The WT K_{D} (252 nM) agrees well with published data (208 nM from SPR, 535 nM from calorimetry) (Okazaki et al., 2004 J Mol Biol 336:1239-49, hereby entirely incorporated by reference). K_{D}'s of the I332E (30 nM) and S239D/I332E (2 nM) variants indicate approximately one- and two- logs greater affinity to V158 FcγRIIIa respectively.

To investigate the capacity of antibodies comprising the Fc variants of the present invention to carry out FcγR-mediated effector function, in vitro cell-based ADCC assays were run using human PBMCs as effector cells. ADCC was measured by the release of lactose dehydrogenase using a LDH Cytotoxicity Detection Kit (Roche Diagnostic). Human PBMCs were purified from leukopacks using a ficoll gradient, and the CD20+ target lymphoma cell line WIL2-S was obtained from ATCC. Target cells were seeded into 96-well plates at 10,000 cells/well, and opsonized using Fc variant or WT antibodies at the indicated final concentration. Triton X100 and PBMCs alone were run as controls. Effector cells were added at 25:1 PBMC5:target cells, and the plate was incubated at 37° C. for 4 hrs. Cells were incubated with the LDH reaction mixture, and fluorescence was measured using a Fusion™ Alpha-FP (Perkin Elmer). Data were normalized to maximal (triton) and minimal (PBMCs alone) lysis, and fit to a sigmoidal dose-response model. FIG. 82 provides these data for select Fc variant antibodies in the context of the variable region PRO70769 and either IgG1 or IgG(1/2) ELLGG. The Fc variants provide clear enhancements in FcγR-mediated CD20+ target cell lysis relative to the WT PRO70769 IgG1 antibody.

These in vitro assays suggest that the Fc variants of the present invention may provide enhanced potency and/or efficacy in a clinical setting. In vivo performance may be affected by a number of factors, including some of which are not considered by these in vitro experiments. One such parameter is the high concentration of non-specific IgG in serum, which has been shown to impact antibody clinical potency (Vugmeyster & Howell, 2004, Int Immunopharmacol 4:1117-24; Preithner et al., 2005, Mol Immunol, 43(8):1183-93, all hereby entirely incorporated by reference). In order to investigate how the Fc variants of the present invention perform in a solution more closely mimicking in vivo biology, the ADCC assays were repeated in the presence of a biologically relevant (1 mg/ml) concentration of IgG purified from human serum (purchased commercially from Jackson Immunoresearch Lab, Inc.). These data are provided in FIG. 83. The efficacy of the WT anti-CD20 antibody is not only reduced, but completely ablated in the presence of serum level IgG. In contrast, the Fc variant antibodies, although significantly reduced, still show substantial capacity to mediate killing against the target cell line.

### Example 14. Fc Variants with Enhanced Complement-Mediated Effector Function

A number of variants were designed with the goal of enhancing complement dependant cytotoxicity (CDC). In the same way that Fc/FcγR binding mediates ADCC, Fc/C1q binding mediates complement dependent cytotoxicity (CDC). There is currently no structure available for the Fc/C1q complex; however, mutagenesis studies have mapped the binding site on human IgG for C1q to a region centered on residues D270, K322, P329, and P331 (Idusogie et al., 2000, J Immunol 164:4178-4184; Idusogie et al., 2001, J Immunol 166:2571-2575, both hereby entirely incorporated by reference). FIG. 84 shows a structure of the human IgG1 Fc region with this epicenter mapped. Select amino acid modifications disclosed in U.S. Ser. No. 10/672,280, U.S. Ser. No. 10/822,231, U.S. Ser. No. 11/124,620, and U.S. Ser. No. 11/256,060, all hereby entirely incorporated by reference, that are structurally proximal to these four residues were investigated to explore variants that may mediate increased affinity for C1q and and/or provide enhanced CDC. Variants that previously showed enhanced FcγR affinity and FcγR-mediated effector function were included in this set of variants to characterize their complement properties. This variant library is provided in FIG. 85.

The variants were constructed as described above in the context of the anti-CD20 antibody PRO70769 (variable region) and either IgG1 or IgG(1/2) ELLGG as the heavy chain constant region. Variants were expressed and purified as described above. A cell-based assay was used to measure the capacity of the Fc variants to mediate CDC. Lysis was measured using release of Alamar Blue to monitor lysis of Fc variant and WT PRO70769-opsonized WIL2-S lymphoma cells by human serum complement. Target cells were washed 3× in 10% FBS medium by centrifugation and resuspension, and WT or variant rituximab antibody was added at the indicated final concentrations. Human serum complement (Quidel) was diluted 50% with medium and added to antibody-opsonized target cells. Final complement concentration was ⅙^{th} original stock. Plates were incubated for 2 hrs at 37° C., Alamar Blue was added, cells were cultured for two days, and fluorescence was measured. Representative data from this assay are shown in FIG. 86. The binding data were normalized to the maximum and minimum luminescence signal for each particular curve, provided by the baselines at low and high antibody concentrations respectively. The data were fit to a sigmoidal dose-response with variable slope model using nonlinear regression, and these fits are represented by the curves in the figure. These fits provide the effective concentration 50% (EC50) (i.e., the concentration required for 50% response) for each antibody, enabling the relative binding affinities of Fc variants to be quantitatively determined. By dividing the EC50 for each variant by that of WT PRO70769, the fold-enhancement or reduction relative to WT PRO70769 (Fold WT) were obtained. These values are provided in FIG. 85. Here a fold above 1 indicates an enhancement in CDC EC50, and a fold below 1 indicates a reduction in CDC EC50 relative to WT PRO70769.

The data in FIGS. 85 and 86 indicate that a number of modifications provide enhanced CDC relative to WT PRO70769 IgG1. For example, greater than 2-fold CDC enhancement is observed for modifications 239D, 267D, 267Q, 268D, 268E, 268F, 268G, 272I, 276D, 276L, 276S, 278R, 282G, 284T, 285Y, 293R, 300T, 324I, 324T, 324V, 326E, 326T, 326W, 327D, 330H, 330S, 332E, 333F, 334T, and 335D (FIG. 15). Additionally, the data show that a number of modifications provide reduced CDC relative to WT PRO70769 IgG1. For example, modifications that show 0.5 fold and lower relative CDC include 235D, 239D, 284D, 322H, 322T, 322Y, 327R, 330E, 330I, 330L, 330N, 330V, 331D, and 331L, 332E (FIG. 15). These modifications provide further valuable structure activity relationship (SAR) information that may be used to guide further design of variants for enhanced CDC. Together the data suggest that modification at positions 235, 239, 267, 268, 272, 276, 278, 282, 284, 285, 293, 300, 322, 324, 326, 327, 330, 331, 332, 333, 334, and 335 (FIG. 15) may provide enhanced CDC relative to a parent Fc polypeptide.

### Example 15. Fc Variants with Reduced FcγR- and Complement-Mediated Effector Function

As described above, in contrast antibody therapeutics and indications wherein effector functions contribute to clinical efficacy, for some antibodies and clinical applications it may be favorable to reduce or eliminate binding to one or more FcγRs, or reduce or eliminate one or more FcγR- or complement-mediated effector functions including but not limited to ADCC, ADCP, and/or CDC. This is often the case for therapeutic antibodies whose mechanism of action involves blocking or antagonism but not killing of the cells bearing target antigen. In these cases depletion of target cells is undesirable and can be considered a side effect. Effector function can also be a problem for radiolabeled antibodies, referred to as radioconjugates, and antibodies conjugated to toxins, referred to as immunotoxins. These drugs can be used to destroy cancer cells, but the recruitment of immune cells via Fc interaction with FcγRs brings healthy immune cells in proximity to the deadly payload (radiation or toxin), resulting in depletion of normal lymphoid tissue along with targeted cancer cells.

A previously unconsidered advantage of ablated FcγR- and complement-binding is that in cases where effector function is not needed, binding to FcγR and complement may effectively reduce the active concentration of drug. Binding to Fc ligands may localize an antibody or Fc fusion to cell surfaces or in complex with serum proteins wherein it is less active or inactive relative to when it is free (uncomplexed). This may be due to decreased effective concentration at binding sites where the antibody is desired, or perhaps Fc ligand binding may put the Fc polypeptide in a conformation in which it is less active than it would be if it were unbound. An additional consideration is that FcγR-receptors may be one mechanism of antibody turnover, and can mediate uptake and processing by antigen presenting cells such as dendritic cells and macrophages. This may affect the pharmacokinetics (or in vivo half-life) of the antibody or Fc fusion and its immunogenicity, both of which are critical parameters of clinical performance.

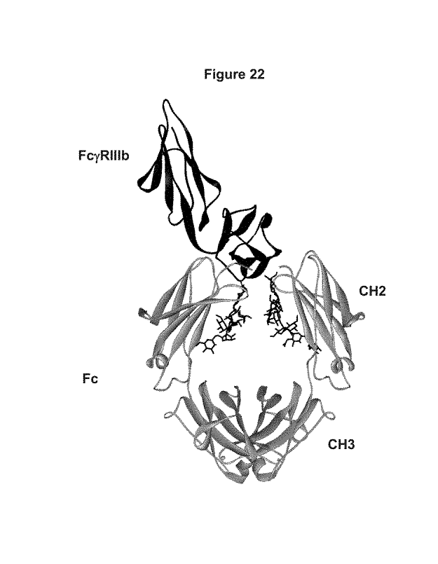

Visual inspection of the Fc/FcγR structure (FIG. 22) and the aforedescribed Fc/C1q interface (FIG. 84), as well as data disclosed above and in U.S. Ser. No. 10/672,280, U.S. Ser. No. 10/822,231, U.S. Ser. No. 11/124,620, and U.S. Ser. No. 11/256,060, all hereby entirely incorporated by reference, were used to guide the design of a library to screen for variants with reduced affinity for FcγRs and reduced CDC. This variant library is provided in FIG. 88. The variants were constructed in the context of PRO70769 IgG1, and expressed and purified as described above. Relative FcγR affinity was measured using the competition AlphaScreen assay, as described above. FIG. 89 shows AlphaScreen data for binding of select Fc variants to human V158 FcγRIIIa, and FIG. 88 provides their Fold IC50's relative to WT PRO70769 IgG1. The variants were also investigated for their capacity to mediate complement-mediated lysis against CD20+WIL2-S lymphoma target cells using the CDC assay described above. FIG. 90 provides CDC data for select Fc variants, and FIG. 88 provides their Fold EC50's relative to WT PRO70769 IgG1. Based on the results of these experiments, select Fc variants were characterized for their capacity to mediate FcγR-mediated effector function. An ADCC assay using human PBMCs as effector cells and WIL2-S lymphoma cells as target cells was carried out as described above. FIG. 91 shows these ADCC data for select variants.

The data indicate that modification at a number of positions provide reduced or ablated FcγR affinity, reduced FcγR-mediated effector function, and reduced complement-mediated effector function. Furthermore, modifications at some positions, including but not limited to 235 and 330, may provide reduced CDC but WT FcγR affinity. For example 235D, 330L, 330N, and 330R display such behavior. Alternatively, modification at some positions, including but not limited to 236 and 299, may provide reduced FcγR affinity but WT level CDC. For example 236I and 299A show these properties.

Based on the results of these experiments, a number of modifications that simultaneously ablate FcγR affinity and CDC were combined in multiple mutations variants in a new library of Fc variants was designed to screen for variants with completely ablated FcγR affinity, FcγR-mediated effector function, and complement-mediated effector function. These variants include modifications at positions 234, 235, 236, 267, 269, 325, and 328, and are provided in FIG. 92. Included in the set are the WT IgG1 antibody, as well as IgG2 and IgG4 antibody versions, an aglycosylated variant N297S, and two variants previously characterized as having reduced effector function: L234A/L235A (Xu et al., 2000, Cellular Immunology 200:16-26; U.S. Ser. No. 10/267,286, hereby entirely incorporated by reference) and E233P/L234V/L235A/G236- (Armour et al., 1999, Eur J Immunol 29:2613-2624, hereby entirely incorporated by reference).

These variants were constructed in the context of the anti-CD20 antibody PRO70769, with the heavy chain constant region IgG1 except for the IgG2 and IgG4 antibodies. Antibodies were expressed and purified as described previously. The competition AlphaScreen assay was used as described previously to measure the relative FcγR affinity of the Fc variants. FIG. 93 shows AlphaScreen data for binding of select variants to the low affinity human activating receptor V158 FcγRIIIa, as well as the high affinity human activating receptor FcγRI. The fold IC50's relative to WT are provided in FIG. 92. Because of its greater binding affinity for the Fc region, FcγRI provides a more stringent test for the variants. The data in FIGS. 92 and 93 support this, showing that although variants may substantially reduce or completely ablate affinity to FcγRIIIa, FcγRI binding is more modestly affected. The Fc variants were also tested for their capacity to mediate complement-mediated lysis against CD20+WIL2-S cells using the CDC assay described above. FIG. 94 shows CDC data for select Fc variants, and FIG. 92 provides the fold EC50's relative to WT PRO70769 IgG1.

In order to investigate the capacity of the Fc variants to mediate ADCC, select variants were subcloned into the anti-Her2/neu antibody trastuzumab (variable region sequences provided in FIGS. 19c and 19d). Trastuzumab robustly provides a substantial signal in ADCC assays against Her2+ expressing cell lines, and therefore provides a stringent test of the Fc variants for reducing/ablating effector function. Fc variants L235G, G236R, G237K, N325L, N325A, L328R, L235G/G236R, G236R/G237K, G236R/N325L, G236R/L328R, G237K/N325L, L235G/G236R/G237K, and G236R/G237K/L328R were constructed in the context of trastuzumab IgG1. WT IgG1,WT IgG2, and WT IgG4 antibody versions were constructed as well. An ADCC assay was carried out as described above, except the Her2+ breast carcinoma cell line SkBr-3 was used as target cells. FIG. 95 provides the results of the ADCC experiments. The data indicate that some of the variants completely ablate ADCC. Additionally, although IgG2 also appears to mediate no ADCC, IgG4 does show a significant level of ADCC.

The results show that amino acid modifications at a number of positions, including but not limited to 232, 234, 235, 236, 237, 238, 239, 265, 267, 269, 270, 297, 299, 325, 327, 328, 329, 330, and 331, provide promising candidates for improving the clinical properties of antibodies and Fc fusions wherein FcγR binding, FcγR-mediated effector functions, and/or complement-mediated effector function are undesired. For example the amino acid modifications 232G, 234G, 234H, 235D, 235G, 235H, 236I, 236N, 236P, 236R, 237K, 237L, 237N, 237P, 238K, 239R, 265G, 267R, 269R, 270H, 297S, 299A, 299I, 299V, 325A, 325L, 327R, 328R, 329K, 330I, 330L, 330N, 330P, 330R, 330S, and 331L provide significantly reduced Fc ligand binding properties and/or effector function. Particularly effective at reducing binding to Fc ligands and effector function are variants 236R/237K, 236R/325L, 236R/328R, 237K/325L, 237K/328R, 325L/328R, 235G/236R, 267R/269R, 234G/235G, 236R/237K/325L, 236R/325L/328R, 235G/236R/237K, and 237K/325L/328R. Notably, the amino acid modifications that compose these variants, including 234G, 235G, 236R, 237K, 267R, 269R, 325L, and 328R, are capable of reducing binding to both FcγRIIIa and FcγR1, and reducing CDC by greater than 10 fold. Additionally, the data show that human IgG2 has significantly reduced FcγR-affinity, FcγR-mediated effector function, and complement-mediated effector function relative to human IgG4.

As discussed above, reduced FcγR affinity and/or effector function may be optimal for Fc polypeptides for which Fc ligand binding or effector function leads to toxicity and/or reduced efficacy. For example, antibodies that target CTLA-4 block inhibition of T-cell activation and are effective at promoting anti-tumor immune response, but destruction of T cells via antibody mediated effector functions may be counterproductive to mechanism of action and/or potentially toxic. Indeed toxicity has been observed with clinical use of the anti-CTLA-4 antibody ipilimumab (Maker et al., 2005, Ann Surg Oncol 12:1005-16, hereby entirely incorporated by reference). The sequences for the anti-CTLA-4 antibody ipilimumab (Mab 10D.1, MDX010) are provided in FIG. 19, taken from U.S. Pat. No. 6,984,720 SEQ ID NO: 5 (VL, FIG. 19e) and SEQ ID NO: 6 (VH, FIG. 19f), hereby entirely incorporated by reference. For illustration purposes, a number of Fc variants of the present invention have been incorporated into the sequence of an antibody targeting CTLA-4. Because combinations of Fc variants of the present invention have typically resulted in additive or synergistic binding modulations, and accordingly additive or synergistic modulations in effector function, it is anticipated that as yet unexplored combinations of the Fc variants provided in the present invention, or with other previously disclosed modifications, will also provide favorable results. Potential Fc variants are provided in FIG. 96a. The optimized antibody sequences comprise at least one non-WT amino acid selected from the group consisting of X₁, X₂, X₃, X₄, X₅, X₆, X₇, and X₈. For example, an improved anti-CTLA-4 antibody sequence comprising the L235G and G236R modifications in the IgG1 constant region are provided in FIGS. 96b and 96c. Alternatively, as the present invention shows, IgG2 and IgG4 can also be used to reduce Fc ligand binding and Fc-mediated effector function. FIGS. 96b and 96d provide the sequences of improved anti-CTLA-4 IgG2 antibody sequences. The use of an anti-CTLA-4 here is solely an example, and is not meant to constrain application of the Fc variants to this antibody or any other particular Fc polypeptide. Other exemplary applications for reduced Fc ligand binding and/or effector function include but are not limited to anti-TNFa antibodies, including for example infliximab and adalimumab, anti-VEGF antibodies, including for example bevacizumab, anti-a4-integrin antibodies, including for example natalizumab, and anti-CD32b antibodies, including for example those described in U.S. Ser. No. 10/643,857, hereby entirely incorporated by reference.

### Example 16: Molecular Biology and Protein Expression/Purification

Experimentation on various Fc variants was carried out in the context of the anti-cancer antibody alemtuzumab (Campath®, a registered trademark of Ilex Pharmaceuticals LP). Alemtuzumab binds a short linear epitope within its target antigen CD52 (Hale et al., 1990, Tissue Antigens 35:118-127; Hale, 1995, Immunotechnology 1:175-187). Alemtuzumab was chosen as an engineering template for its efficacy due in part to its ability to recruit effector cells (Dyer et al., 1989, Blood 73:1431-1439; Friend et al., 1991, Transplant Proc 23:2253-2254; Hale et al., 1998, Blood 92:4581-4590; Glennie et al., 2000, Immunol Today 21:403-410), and because production and use of its antigen in binding assays are relatively straightforward. In order to evaluate the optimized Fc variants of the present invention in the context of other antibodies, select Fc variants were evaluated in the anti-Her2 antibody trastuzumab (Herceptin®, a registered trademark of Genentech), the anti-CD20 antibody rituximab (Rituxan®, a registered trademark of IDEC Pharmaceuticals Corporation), the anti-EGFR antibody cetuximab (Erbitux®, a registered trademark of Imclone), and the anti-CD20 antibody PRO70769 (PCT/US2003/040426, entitled “Immunoglobulin Variants and Uses Thereof”). The use of alemtuzumab, trastuzumab, rituximab, cetuximab, and PRO70769 for screening purposes is not meant to constrain the present invention to any particular antibody.

The IgG1 full length light (V_{L}-C_{L}) and heavy (V_{H}-Cγ1-Cγ2-Cγ3) chain antibody genes for alemtuzumab (campath-1H, James et al., 1999, J Mol Biol 289: 293-301), trastuzumab (hu4D5-8; Carter et al., 1992, Proc Natl Acad Sci USA 89:4285-4289; Gerstner et al., 2002, J Mol. Biol., 321: 851-862), rituximab (C2B8, U.S. Pat. No. 6,399,061), and cetuximab (C225, PCT US96/09847) were constructed using recursive PCR with convenient end restriction sites to facilitate subcloning. The genes were ligated into the mammalian expression vector pcDNA3.1Zeo (Invitrogen), comprising the full length light kappa (Cκ) and heavy chain IgG1 constant regions. The V_{H}-Cγ1-Cγ2-Cγ3 clone in pcDNA3.1zeo was used as a template for mutagenesis of the Fc region. Mutations were introduced into this clone using PCR-based mutagenesis or quick-change mutagenesis (Stratagene) techniques. Fc variants were sequenced to confirm the fidelity of the sequence. Plasmids containing heavy chain gene (V_{H}-Cγ1-Cγ2-Cγ3) (wild-type or variants) were co-transfected with plasmid containing light chain gene (V_{L}-C_{L}) into 293T cells. Media were harvested 5 days after transfection. Expression of immunoglobulin was monitored by screening the culture supernatant of transfectomas by western using peroxidase-conjugated goat-anti human IgG (Jackson ImmunoResearch, catalog #109-035-088). FIG. 98 shows expression of wild-type alemtuzumab and variants 1 through 10 in 293T cells. Antibodies were purified from the supernatant using protein A affinity chromatography (Pierce, Catalog #20334. FIG. 99 shows results of the protein purification for WT alemtuzumab. Antibody Fc variants showed similar expression and purification results to WT. Some Fc variants were deglycosylated in order to determine their solution and functional properties in the absence of carbohydrate. To obtain deglycosylated antibodies, purified alemtuzumab antibodies were incubated with peptide-N-glycosidase (PNGase F) at 37° C. for 24h. FIG. 100 presents an SDS PAGE gel confirming deglycosylation for several Fc variants and WT alemtuzumab.

In order to confirm the functional fidelity of alemtuzumab produced under these conditions, the antigenic CD52 peptide, fused to GST, was expressed in E. coli BL21 (DE3) under IPTG induction. Both un-induced and induced samples were run on a SDS PAGE gel, and transferred to PVDF membrane. For western analysis, either alemtuzumab from Sotec (final concentration 2.5 ng/ul) or media of transfected 293T cells (final alemtuzumab concentration about 0.1-0.2 ng/ul) were used as primary antibody, and peroxidase-conjugated goat-anti human IgG was used as secondary antibody. FIG. 101 presents these results. The ability to bind target antigen confirms the structural and functional fidelity of the expressed alemtuzumab. Fc variants that have the same variable region as WT alemtuzumab are anticipated to maintain a comparable binding affinity for antigen.

The gene encoding the extracellular region of human V158 FcγRIIIa was obtained by PCR from a clone obtained from the Mammalian Gene Collection (MGC:22630). F158 FcγRIIIa was constructed by mutagenesis of the V158 FcγRIIIa gene. The genes encoding the extracellular regions of human FcγRI, human FcγRIIa, human FcγRIIb, human FcγRIIc, mouse FcγRIII, and human FcRn a chain and P-microglobulin chain were constructed using recursive PCR. FcγRs and FcRn a chain were fused at the C-terminus with a 6×His-tag and a GST-tag.

All genes were subcloned into the pcDNA3.1zeo vector. For expression, vectors containing human FcγRs were transfected into 293T cells, FcRn α chain and β-microglobulin chain were co-transfected into 293T cells, and mouse FcγRIII was transfected into NIH3T3 cells. Media containing secreted receptors were harvested 3 days later and purified using Nickel affinity chromatography. For western analysis, membrane was probed with an anti-GST antibody. FIG. 102 presents an SDS PAGE gel that shows the results of expression and purification of human V158 FcγRIIIa. Purified human C1q protein complex was purchased commercially (Quidel Corp., San Diego).

### Example 17. Fc Ligand Binding Assays

Binding to the human Fc ligands FcγRI, FcγRIIa, FcγRIIb, FcγRIIc, FcγRIIIa, C1q, and FcRn was measured for the designed Fc variants. Binding affinities were measured using an AlphaScreen™ assay (Amplified Luminescent Proximity Homogeneous Assay (ALPHA), PerkinElmer, Wellesley, Mass.), a bead-based luminescent proximity assay. Laser excitation of a donor bead excites oxygen, which if sufficiently close to the acceptor bead generates a cascade of chemiluminescent events, ultimately leading to fluorescence emission at 520-620 nm. WT alemtuzumab antibody was biotinylated by standard methods for attachment to streptavidin donor beads, and GST-tagged FcγRs and FcRn were bound to glutathione chelate acceptor beads. For the C1q binding assay, untagged C1q protein was conjugated with Digoxygenin (DIG, Roche) using N-hydrosuccinimide (NHS) chemistry and bound to DIG acceptor beads. For the protein A binding assay, protein A acceptor beads were purchased directly from PerkinElmer. The AlphaScreen assay was applied as a competition assay for screening Fc variants. In the absence of competing Fc variants, WT antibody and FcγR interact and produce a signal at 520-620 nm. Addition of untagged Fc variant competes with the WT Fc/FcγR interaction, reducing fluorescence quantitatively to enable determination of relative binding affinities. Fc variants were screened in the context of either alemtuzumab or trastuzumab, and select Fc variants were also screened in the context of rituximab and cetuximab.

FIG. 103 shows AlphaScreen data for binding to human V158 FcγRIIIa by select Fc variants. The binding data were normalized to the maximum and minimum luminescence signal for each particular curve, provided by the baselines at low and high antibody concentrations respectively. The data were fit to a one site competition model using nonlinear regression, and these fits are represented by the curves in the figure. These fits provide the inhibitory concentration 50% (IC50) (i.e., the concentration required for 50% inhibition) for each antibody, illustrated by the dotted lines in FIG. 103, thus enabling the relative binding affinities of Fc variants to be quantitatively determined. By dividing the IC50 for each variant by that of WT alemtuzumab, the fold- enhancement or reduction relative to WT Herceptin (Fold WT) are obtained. Here, WT alemtuzumab has an IC50 of (4.63×10⁻⁹)×(2)=9.2 nM, whereas S239D has an IC50 of (3.98×10⁻¹⁰)×(2)=0.8 nM. Thus, S239D alemtuzumab binds 9.2 nM/0.8 nM=11.64-fold more tightly than WT alemtuzumab to human V158 FcγRIIIa. FIGS. 104a and 104b provide AlphaScreen data showing additional Fc variants, with substitutions at positions 239, 264, 272, 274, and 332, that bind more tightly to FcγRIIIa, and thus are candidates for improving the effector function of Fc polypeptides.

Fc variants were also screened in parrallel for other Fc ligands. As discussed, the inhibitory receptor FcγRIIb plays an important role in effector function. Exemplary data for binding of select Fc variants of the invention to human FcγRIIb, as measured by the AlphaScreen, are provided in FIG. 105. FcγRIIa is an activating receptor that is highly homologous to FcγRIIb. Exemplary data for binding of select Fc variants to the R131 polymorphic form of human FcγRIIa are provided in FIG. 106. Another important Fc ligand is the neonatal Fc receptor FcRn. As discussed, this receptor binds to the Fc region between the Cγ2 and Cγ3 domains; because binding mediates endosomal recycling, affinity of Fc for FcRn is a key determinant of antibody and Fc fusion pharmacokinetics. Exemplary data showing binding of select Fc variants to FcRn, as measured by the AlphaScreen, are provided in FIG. 107. The binding site for FcRn on Fc, between the Cγ2 and Cγ3 domains, is overlapping with the binding site for bacterial proteins A and G. Because protein A is frequently employed for antibody purification, select variants were tested for binding to this Fc ligand. FIG. 108 provides these AlphaScreen data. Although protein A was not included in the parrallel screen for all variants, the ability of the Fc variants to be purified using protein A chromatography (see Example 16) implies that for the majority of Fc variants the capacity to bind protein A, and moreover the integrity of the Cγ2-Cγ3 hinge region, are unaffected by the Fc substitutions.

The data for binding of Fc variants to FcγRI, FcγRIIa, FcγRIIb, FcγRIIc, FcγRIIIa, C1q, and FcRn were analyzed as described above for FIG. 104. The fold-enhancement or reduction relative to WT for binding of each variant to each Fc ligand, as measured by the AlphaScreen, are provided in FIG. 24. The table presents for each variant the variant number (Variant), the substitution(s) of the variant, the antibody context (Context), the fold affinity relative to WT (Fold) and the confidence (Conf) in the fold affinity for binding to each Fc ligand, and the IIIa:IIb specificity ratio (IIIa:IIb) (see below). Multiple data sets were acquired for many of the variants, and all data for a given variant are grouped together. The context of the antibody indicates which antibodies have been constructed with the particular Fc variant; a=alemtuzumab, t=trastuzumab, r=rituximab, c=cetuximab, and p=PRO70769. The data provided were acquired in the context of the first antibody listed, typically alemtuzumab, although in some cases trastuzumab. An asterix (*) indicates that the data for the given Fc ligand was acquired in the context of trastuzumab. A fold (Fold) above 1 indicates an enhancement in binding affinity, and a fold below 1 indicates a reduction in binding affinity relative to the parent antibody for the given Fc ligand. Confidence values (Conf) correspond to the log confidence levels, provided from the fits of the data to a sigmoidal dose response curve. As is known in the art, a lower Conf value indicates lower error and greater confidence in the Fold value. The lack of data for a given variant and Fc ligand indicates either that the fits to the data did not provide a meaningful value, or that the variant was not tested for that Fc ligand.

FIG. 24 shows that a number of Fc variants have been obtained with enhanced affinities and altered specificities for the various Fc ligands. Some Fc variants of the present invention provide selective enhancement in binding affinity to different Fc ligands, whereas other provide selective reduction in binding affinity to different Fc ligands. By “selective enhancement” as used herein is meant an improvement in or a greater improvement in binding affinity of an Fc variant to one or more Fc ligands relative to one or more other Fc ligands. For example, for a given variant, the Fold WT for binding to, say FcγRIIa, may be greater than the Fold WT for binding to, say FcγRIIb. By “selective reduction” as used herein is meant a reduction in or a greater reduction in binding affinity of an Fc variant to one or more Fc ligands relative to one or more other Fc ligands. For example, for a given variant, the Fold WT for binding to, say FcγRI, may be lower than the Fold WT for binding to, say FcγRIIb. As an example of such selectivity, G236S provides a selective enhancement to FcγRII's (IIa, IIb, and IIc) relative to FcγRI and FcγRIIIa, with a somewhat greater enhancement to FcγRIIa relative to FcγRIIb and FcγRIIc. G236A, however, is highly selectively enhanced for FcγRIIa, not only with respect to FcγRI and FcγRIIIa, but also over FcγRIIb and FcγRIIc. Selective enhancements and reductions are observed for a number of Fc variants, including but not limited to variants comprising substitutions at residues L234, L235, G236, S267, H268, R292, E293, Q295, Y300, S324, A327, L328, A330, and T335. Overall, the data provided in FIG. 24 show that it is indeed possible to tune the Fc region for Fc ligand specificity, often by using very subtle mutational differences, despite the fact that a number of highly homologous receptors bind to the same FcγR binding site. The present invention provides a number of Fc variants that may be used to selectively enhance, as well as selectively reduce, affinity of an Fc polypeptide for certain Fc ligands relative to others. Collections of Fc variants such as these will not only enable the generation of antibodies and Fc fusions that have effector function tailored for the desired outcome, but they also provide a unique set of reagents with which to experimentally investigate and characterize effector function biology.

As discussed, optimal effector function may result from Fc variants wherein affinity for activating FcγRs is greater than affinity for the inhibitory FcγRIIb. Indeed a number of Fc variants have been obtained that show differentially enhanced binding to FcγRIIIa over FcγRIIb. AlphaScreen data directly comparing binding to FcγRIIIa and FcγRIIb for two Fc variants with this specificity profile, A330L and A330Y, are shown in FIGS. 109a and 109b. This concept can be defined quantitatively as the fold-enhancement or -reduction of the activating FcγRIIIa divided by the fold-enhancement or -reduction of the inhibitory FcγRIIb, herein referred to as the “FcγRIIIa-fold:FcγRIIb-fold ratio” or “IIIa:IIb ratio”. This value is provided in the last column of FIG. 24 (as IIIa:IIb). Combination of A330L and A330Y with other variants, for example A330L/I332E, A330Y/I332, and S239D/A330L/I332E, provide very favorable IIIa:IIb ratios. FIG. 24 shows that a number of Fc variants provide a positive, favorable FcγRIIIa to FcγRIIb specificity profile, with a IIIa:IIb ratio as high as 86:1.

Some of the most promising Fc variants of the present invention for enhancing effector function have both substantial increases in affinity for FcγRIIIa and favorable FcγRIIIa-fold:FcγRIIb-fold ratios. These include, for example, S239D/I332E (FcγRIIIa-fold=56-192, FcγRIIIa-fold:FcγRIIb-fold=3), S239D/A330Y/I332E (FcγRIIIa-fold=130), S239D/A330L/I332E (FcγRIIIa-fold=139, FcγRIIIa-fold:FcγRIIb-fold=18), and S239D/S298A/I332E (FcγRIIIa-fold=295, FcγRIIIa-fold:FcγRIIb-fold=48). FIGS. 110a-110c show AlphaScreen data monitoring binding of these and other Fc variants in the context of trastuzumab to human V158 FcγRIIIa and human FcγRIIb.

In addition to alemtuzumab and trastuzumab, select Fc variants were screened in the context of other antibodies in order to investigate the breadth of their applicability. AlphaScreen data measuring binding of select Fc variants to human V158 FcγRIIIa in the context of the anti-CD20 antibody rituximab[and], the anti-CD20 antibody PRO70769, and the anti-EGFR antibody cetuximab are shown in FIG. 111, FIGS. 112, and 135, respectively. Together with the data shown previously for alemtuzumab and trastuzumab, the results indicate consistent binding enhancements regardless of the antibody context, and thus that the Fc variants of the present invention are broadly applicable to antibodies and Fc fusions.

As discussed above, an important parameter of Fc-mediated effector function is the affinity of Fc for both V158 and F158 polymorphic forms of FcγRIIIa. AlphaScreen data comparing binding of select variants to the two receptor allotypes are shown in FIG. 113a (V158 FcγRIIIa) and FIG. 113b (F158 FcγRIIIa). As can be seen, all variants improve binding to both FcγRIIIa allotypes. These data indicate that those Fc variants of the present invention with enhanced effector function will be broadly applicable to the entire patient population, and that enhancement to clinical efficacy will potentially be greatest for the low responsive patient population who need it most.

The FcγR binding affinities of these Fc variants were further investigated using Surface Plasmon Resonance (SPR) (Biacore, Uppsala, Sweden). SPR is a sensitive and extremely quantitative method that allows for the measurement of binding affinities of protein-protein interactions, and has been used to effectively measure Fc/FcγR binding (Radaev et al., 2001, J Biol Chem 276:16478-16483). SPR thus provides an excellent complementary binding assay to the AlphaScreen assay. His-tagged V158 FcγRIIIa was immobilized to an SPR chip, and WT and Fc variant alemtuzumab antibodies were flowed over the chip at a range of concentrations. Binding constants were obtained from fitting the data using standard curve-fitting methods. Table 3 presents dissociation constants (Kd) for binding of select Fc variants to V158 FcγRIIIa and F158 FcγRIIIa obtained using SPR, and compares these with IC50s obtained from the AlphaScreen assay. By dividing the Kd and IC50 for each variant by that of WT alemtuzumab, the fold-improvements over WT (Fold WT) are obtained.

TABLE 3

| | SPR | | SPR | | | | | |
|---|---|---|---|---|---|---|---|---|
| | V158 | | F158 | | AlphaScreen | | AlphaScreen | |
| | FcγRIIIa | | FcγRIIIa | | V158 FcγRIIIa | | F158 FcγRIIIa | |
| | Kd | FOLD | Kd | FOLD | IC50 | FOLD | IC50 | FOLD |
| | (nM) | WT | (nM) | WT | (nM) | WT | (nM) | WT |
| WT | 68 | | 730 | | 6.4 | | 17.2 | |
| V264I | 64 | 1.1 | 550 | 1.3 | 4.5 | 1.4 | 11.5 | 1.5 |
| I332E | 31 | 2.2 | 72 | 10.1 | 1.0 | 6.4 | 2.5 | 6.9 |
| V264I/I332E | 17 | 4.0 | 52 | 14.0 | 0.5 | 12.8 | 1.1 | 15.6 |
| S298A | 52 | 1.3 | 285 | 2.6 | 2.9 | 2.2 | 12.0 | 1.4 |
| S298A/E333A/K334A | 39 | 1.7 | 156 | 4.7 | 2.5 | 2.6 | 7.5 | 2.3 |

The SPR data corroborate the improvements to FcγRIIIa affinity observed by AlphaScreen assay. Table 3 further indicates the superiority of V264I/I332E and I332E over S298A and S298A/E333A/K334A; whereas S298A/E333A/K334A improves Fc binding to V158 and F158 FcγRIIIa by 1.7-fold and 4.7-fold respectively, I332E shows binding enhancements of 2.2-fold and 10.1-fold respectively, and V264I/I332E shows binding enhancements of 4.0-fold and 14-fold respectively. Also worth noting is that the affinity of V264I/I332E for F158 FcγRIIIa (52 nM) is better than that of WT for the V158 allotype (68 nM), suggesting that this Fc variant, as well as those with even greater improvements in binding, may enable the clinical efficacy of antibodies for the low responsive patient population to achieve that currently possible for high responders. The correlation between the SPR and AlphaScreen binding measurements are shown in FIGS. 114a-114d. FIGS. 114a and 114b show the Kd-IC50 correlations for binding to V158 FcγRIIIa and F158 FcγRIIIa respectively, and FIGS. 114c and 114d show the fold-improvement correlations for binding to V158 FcγRIIIa and F158 FcγRIIIa respectively. The good fits of these data to straight lines (r²=0.9, r²=0.84, r²=0.98, and r²=0.90) support the accuracy the AlphaScreen measurements, and validate its use for determining the relative FcγR binding affinities of Fc variants.

Select Fc variants were screened in the context of multiple antibodies in order to investigate the breadth of their applicability. AlphaScreen™ data for binding of select Fc variants to human V158 FcγRIIIa in the context of trastuzumab, rituximab, and cetuximab are shown in FIGS. 138a, 138b, 139a, and 139b. Together with the data for alemtuzumab in FIG. 104, the results indicate consistent binding enhancements regardless of the antibody context, and thus that the Fc variants of the present invention are broadly applicable to antibodies and Fc fusions.

SPR data were also acquired for binding of select trastuzumab Fc variants to human V158 FcγRIIIa, F158 FcγRIIIa, and FcγRIIb. These data are shown in Table 4. The Fc variants tested show substantial binding enhancements to the activating receptor FcγRIIIa, with over 100-fold tighter binding observed for interaction of S239D/I332E/S298A with F158 FcγRIIIa. Furthermore, for the best FcγRIIIa binders, F158 FcγRIIIa/FcγRIIb ratios of 3-4 are observed.

TABLE 4

| | SPR | | SPR | | SPR | |
|---|---|---|---|---|---|---|
| | V158 FcγRIIIa | | F158 FcγRIIIa | | FcγRIIb | |
| | Kd | FOLD | Kd | FOLD | IC50 | FOLD |
| | (nM) | WT | (nM) | WT | (nM) | WT |
| WT | 363.5 | | 503 | | 769 | |
| V264I/I332E | 76.9 | 4.7 | 252 | 2.0 | 756 | 1.0 |
| V264I/I332E/A330L | 113.0 | 3.2 | 88 | 5.7 | 353 | 2.2 |
| S239D/I332E/A330L | 8.2 | 44.3 | 8.9 | 56.5 | 46 | 16.7 |
| S239D/I332E/S298A | 8.7 | 41.8 | 4.9 | 102.7 | 32 | 24.0 |
| S239D/I332E/V264I/ | 12.7 | 28.6 | 6.3 | 79.8 | 35 | 22.0 |
| A330L | | | | | | |

FIG. 140 shows AlphaScreen™ data for binding of select Fc variants to human R131 FcγRIIa. As can be seen, those aforementioned variants with favorable binding enhancements and specificity profiles also show enhanced binding to this activating receptor. The use of FcγRIIIa, FcγRIIb, and FcγRIIc for screening is not meant to constrain experimental testing to these particular FcγRs; other FcγRs are contemplated for screening, including but not limited to the myriad isoforms and allotypes of FcγRI, FcγRII, and FcγRIII from humans, mice, rats, monkeys, and the like, as previously described.

As discussed, although there is a need for greater effector function, for some antibody therapeutics, reduced or eliminated effector function may be desired. Several Fc variants in FIG. 24 substantially reduce or ablate FcγR binding, and thus may find use in antibodies and Fc fusions wherein effector function is undesirable. AlphaScreen data measuring binding of some exemplary Fc variants to human V158 FcγRIIIa are shown in FIGS. 115a and 115b. These Fc variants, as well as their use in combination, may find use for eliminating effector function when desired, for example in antibodies and Fc fusions whose mechanism of action involves blocking or antagonism but not killing of the cells bearing target antigen. Based on the data provided in FIG. 24, preferred positions for reducing Fc ligand binding and/or effector function, that is positions that may be modified to reduce binding to one or more Fc ligands and/or reduce effector function, include but are not limited to positions 232, 234, 235, 236, 237, 239, 264, 265, 267, 269, 270, 299, 325, 328, 329, and 330.

### Example 18. ADCC of Fc Variants

In order to determine the effect on effector function, cell-based ADCC assays were performed on select Fc variants. ADCC was measured using the DELFIA® EuTDA-based cytotoxicity assay (Perkin Elmer, MA) with purified human peripheral blood monocytes (PBMCs) as effector cells. Target cells were loaded with BATDA at 1×10⁶ cells/ml, washed 4 times and seeded into 96-well plate at 10,000 cells/well. The target cells were then opsonized using Fc variant or WT antibodies at the indicated final concentration. Human PBMCs, isolated from buffy-coat were added at the indicated fold-excess of target cells and the plate was incubated at 37° C. for 4 hrs. The co-cultured cells were centrifuged at 500×g, supernatants were transferred to a separate plate and incubated with Eu solution, and relative fluorescence units were measured using a Packard Fusion™ α-FP HT reader (Packard Biosciences, IL). Samples were run in triplicate to provide error estimates (n=3, +/−S.D.). PBMCs were allotyped for the V158 or F158 FcγRIIIa allotype using PCR.

ADCC assays were run on Fc variant and WT alemtuzumab using DoHH-2 lymphoma target cells. FIG. 116a is a bar graph showing the ADCC of these proteins at 10 ng/ml antibody. Results show that alemtuzumab Fc variants I332E, V264I, and I332E/V264I have substantially enhanced ADCC compared to WT alemtuzumab, with the relative ADCC enhancements proportional to their binding improvements to FcγRIIIa as indicated by AlphaScreen assay and SPR. The dose dependence of ADCC on antibody concentration is shown in FIG. 116b. The binding data were normalized to the minimum and maximum fluorescence signal for each particular curve, provided by the baselines at low and high antibody concentrations respectively. The data were fit to a sigmoidal dose-response model using nonlinear regression, represented by the curve in the figure. The fits enable determination of the effective concentration 50% (EC50) (i.e., the concentration required for 50% effectiveness), which provides the relative enhancements to ADCC for each Fc variant. The EC50s for these binding data are analogous to the IC50s obtained from the AlphaScreen competition data, and derivation of these values is thus analogous to that described in Example 17 and FIG. 104. In FIG. 116b, the log(EC50)s, obtained from the fits to the data, for WT, V264I/I332E, and S239D/I332E alemtuzumab are 0.99, 0.60, and 0.49, respectively, and therefore their respective EC50s are 9.9, 4.0, and 3.0. Thus, V264I/I332E and S239E/I332E provide a 2.5-fold and 3.3-fold enhancement respectively in ADCC over WT alemtuzumab using PBMCs expressing heterozygous V158/F158 FcγRIIIa. These data are summarized in Table 5 below.

TABLE 5

| | log(EC50) | EC50 (ng/ml) | Fold WT |
|---|---|---|---|
| WT | 0.99 | 9.9 | |
| V264I/I332E | 0.60 | 4.0 | 2.5 |
| S239D/I332E | 0.49 | 3.0 | 3.3 |

In order to determine whether these AMU enhancements are broadly applicable to antibodies, select Fc variants were evaluated in the context of the anti-Her2 antibody trastuzumab, and the anti-CD20 antibody rituximab. ADCC assays were run on Fc variant and WT trastuzumab using two breast carcinoma target cell lines BT474 and Sk-Br-3. FIG. 117a shows a bar graph illustrating ADCC at 1 ng/ml antibody. Results indicate that V264I and V264I/I332E trastuzumab provide substantially enhanced ADCC compared to WT trastuzumab, with the relative ADCC enhancements proportional to their binding improvements to FcγRIIIa as indicated by AlphaScreen assay and SPR. FIGS. 117b and 117c show the dose dependence of ADCC on antibody concentration for select Fc variants. The EC50s obtained from the fits of these data and the relative fold-improvements in ADCC are provided in Table 6 below. Significant ADCC improvements are observed for I332E trastuzumab when combined with A330L and A330Y. Furthermore, S239D/A330L/I332E provides a substantial ADCC enhancement, greater than 300-fold for PBMCs expressing homozygous F158/F158 FcγRIIIa, relative to WT trastuzumab and S298A/E333A/K334A, consistent with the FcγR binding data observed by the AlphaScreen assay and SPR.

TABLE 6

| | log(EC50) | EC50 (ng/ml) | Fold WT |
|---|---|---|---|
| FIG. 117b | | | |
| WT | 1.1 | 11.5 | |
| I332E | 0.34 | 2.2 | 5.2 |
| A330Y/I332E | −0.04 | 0.9 | 12.8 |
| A330L/I332E | 0.04 | 1.1 | 10.5 |
| FIG. 117c | | | |
| WT | −0.15 | 0.71 | |
| S298A/E333A/K334A | −0.72 | 0.20 | 3.6 |
| S239D/A330L/I332E | −2.65 | 0.0022 | 323 |

ADCC assays were run on V264I/I332E, WT, and S298A/D333A/K334A rituximab using WIL2-S lymphoma target cells. FIG. 118a presents a bar graph showing the ADCC of these proteins at 1 ng/ml antibody. Results indicate that V264I/I332E rituximab provides substantially enhanced ADCC relative to WT rituximab, as well as superior ADCC to S298A/D333A/K334A, consistent with the FcγRIIIa binding improvements observed by AlphaScreen assay and SPR. FIGS. 118b and 118c show the dose dependence of ADCC on antibody concentration for select Fc variants. The EC50s obtained from the fits of these data and the relative fold-improvements in ADCC are provided in Table 7 below. As can be seen S239D/I332E/A330L rituximab provides greater than 900-fold enhancement in EC50 over WT for PBMCs expressing homozygous F158/F158 FcγRIIIa. The differences in ADCC enhancements observed for alemtuzumab, trastuzumab, and rituximab are likely due to the use of different PBMCs, different antibodies, and different target cell lines.

TABLE 7

| | log(EC50) | EC50 (ng/ml) | Fold WT |
|---|---|---|---|
| FIG. 118b | | | |
| WT | 0.23 | 1.7 | |
| S298A/E333A/K334A | −0.44 | 0.37 | 4.6 |
| V264I/I332E | −0.83 | 0.15 | 11.3 |
| FIG. 118c | | | |
| WT | 0.77 | 5.9 | |
| S239D/I332E/A330L | −2.20 | 0.0063 | 937 |

Thus far, ADCC data has been normalized such that the lower and upper baselines of each Fc polypeptide are set to the minimal and maximal fluorescence signal for that specific Fc polypeptide, typically being the fluorescence signal at the lowest and highest antibody concentrations respectively. Although presenting the data in this matter enables a straightforward visual comparison of the relative EC50s of different antibodies (hence the reason for presenting them in this way), important information regarding the absolute level of effector function achieved by each Fc polypeptide is lost. FIGS. 119a, 119b, and 119c present cell-based ADCC data for the anti-Her2 antibody trastuzumab, the anti-CD20 antibody rituximab, and the anti-CD20 antibody PRO70769, respectively that have been normalized according to the absolute minimal lysis for the assay, provided by the fluorescence signal of target cells in the presence of PBMCs alone (no antibody), and the absolute maximal lysis for the assay, provided by the fluorescence signal of target cells in the presence of Triton X1000. The graphs show that the antibodies differ not only in their EC50, reflecting their relative potency, but also in the maximal level of ADCC attainable by the antibodies at saturating concentrations, reflecting their relative efficacy. Thus far, these two terms, potency and efficacy, have been used loosely to refer to desired clinical properties. In the current experimental context, however, they are denoted as specific quantities, and therefore are here explicitly defined. By “potency” as used in the current experimental context is meant the EC50 of an Fc polypeptide. By “efficacy” as used in the current experimental context is meant the maximal possible effector function of an Fc polypeptide at saturating levels. In addition to the substantial enhancements to potency described thus far, FIGS. 119a-c show that the Fc variants of the present invention provide greater than 100% enhancements in efficacy over WT trastuzumab and rituximab.

### Example 119. Cross-Validation of Fc Variants

Select Fc variants were validated for their FcγR binding and ADCC improvements in the context of two antibodies—alemtuzumab and trastuzumab. Binding to human V158 FcγRIIIa was measured using both AlphaScreen and SPR as described above. Exemplary AlphaScreen data measuring FcγRIIIa binding are provided in FIG. 120. ADCC was carried out in the context of trastuzumab using Sk-Br-3 target cells and LDH detection as described above. Exemplary ADCC data are provided in FIG. 121. Table 8 provides a summary of the fold FcγRIIIa binding affinities to relative to WT as determined by AlphaScreen and SPR, and the fold ADCC relative to WT for a series of Fc variants in the context of alemtuzumab (alem) and trastuzumab (trast).

TABLE 8

| Variant | Variant | | Fold WT | V158 | FcγRIIIa |
|---|---|---|---|---|---|
| Substitution | Number | Context | AlphaScreen | SPR | ADCC |
| G236S | 719 | trast | 2.78 | 1.34 | 0.37 |
| G236S | 719 | alem | 6.22 | 6.69 | |
| S239E | 43 | trast | 29.99 | 4.17 | 7.6 |
| S239E | 43 | alem | 2.64 | 3.28 | |
| S239D | 86 | trast | 16.9 | 3.5 | 6.1 |
| S239D | 86 | alem | 36.56 | 16.61 | |
| K246H | 812 | trast | 17.91 | 2.67 | 2 |
| K246H | 812 | alem | 13.58 | 22.36 | |
| K246Y | 813 | trast | 17.44 | 2.39 | 1.36 |
| K246Y | 813 | alem | 4.32 | 7.07 | |
| R255Y | 818 | trast | 21.14 | 2.75 | 1.6 |
| R255Y | 818 | alem | 0.92 | 1.41 | |
| E258H | 820 | trast | 1.18 | 0.77 | 0.76 |
| E258H | 820 | alem | 2.35 | 5.5 | |
| E258Y | 821 | trast | 2.82 | 1.69 | 0.92 |
| E258Y | 821 | alem | 0.64 | 1.77 | |
| T260H | 824 | trast | 35.32 | 2.82 | |
| T260H | 824 | alem | 1 | 1.86 | |
| S267E | 338 | alem | 9.33 | 2.62 | |
| H268D | 350 | trast | 45.27 | 4.76 | 4.59 |
| H268D | 350 | alem | 10.55 | 5.66 | |
| E272I | 237 | trast | 5.86 | 1.63 | 1.38 |
| E272I | 237 | trast | 3.24 | 1.99 | |
| E272R | 634 | alem | | 1.38 | |
| E272H | 636 | trast | 1.02 | 0.65 | 1.28 |
| E272H | 636 | alem | 187.1 | 383.88 | |
| E272P | 642 | trast | 0.005 | 0.522 | 0.39 |
| E272P | 642 | alem | 1.46 | 1.41 | |
| E283H | 839 | trast | 0.99 | 0.71 | 1.4 |
| E283H | 839 | alem | | 2.31 | |
| E283L | 840 | trast | 19.88 | 3.68 | 5.2 |
| E283L | 840 | alem | 1.36 | 2.56 | |
| V284E | 844 | trast | 2.82 | 1.26 | 0.84 |
| V284E | 844 | alem | | 1.51 | |
| E293R | 555 | trast | 1.15 | 0.94 | 0.47 |
| S298D | 364 | trast | 3.48 | 1.49 | 0.58 |
| S304T | 879 | trast | 6.33 | 1.65 | 1.02 |
| S304T | 879 | alem | | 12.85 | |
| S324I | 267 | trast | 5.26 | 1.46 | 2.21 |
| S324G | 608 | trast | 3.04 | 1.76 | 3.23 |
| S324G | 608 | alem | 13.62 | 14.17 | |
| K326E | 103 | trast | 6.12 | 2.12 | 2.87 |
| K326E | 103 | alem | 1.86 | 3.13 | |
| A327D | 274 | trast | 2.44 | 1.31 | 1.04 |
| I332E | 22 | trast | | | |
| I332D | 62 | trast | 19 | 2.57 | 5 |
| I332D | 62 | alem | 21.65 | 11.16 | |
| E333Y | 284 | trast | 8.24 | 1.94 | 2.23 |
| K334I | 285 | trast | 15.24 | 7.1 | 1.2 |
| K334T | 286 | trast | 15.73 | 6.79 | 3.14 |
| K334F | 287 | trast | 10.46 | 5.82 | 1.92 |

### Example 20. ADCC at Varying Target Antigen Expression Levels

A critical parameter governing the clinical efficacy of anti-cancer antibodies is the expression level of target antigen on the surface of tumor cells. Thus, a major clinical advantage of Fc variants that enhance ADCC may be that it enables the targeting of tumors that express lower levels of antigen. In order to test this hypothesis, WT and Fc variant trastuzumab antibodies were tested for their ability to mediate ADCC against different cell lines expressing varying levels of the Her2/neu target antigen using the DELFIA® EuTDA method. Four cell lines cell lines expressing amplified to low levels of Her2/neu receptor were used, including Sk-Br-3 (1×10⁶ copies), SkOV3 (˜1×10⁵), OVCAR3(˜1×10⁴), and MCF-7 (˜3×10³ copies) (FIG. 122a). Target cells were loaded with BATDA in batch for 25 minutes, washed multiple times with medium and seeded at 10,000 cells per well in 96-well plates. Target cells were opsonized for 15 minutes with various antibodies and concentrations (final conc. ranging from 100 ng/ml to 0.0316 ng/ml in ½ log steps, including no treatment control). Human PBMCs, isolated from buffy-coat and allotyped as homozygous F158/F158 FcγRIIIa were then added to opsonized cells at 25-fold excess and co-cultured at 37° C. for 4 hrs. Thereafter, plates were centrifuged, supernatants were removed and treated with Eu3+ solution, and relative fluorescence units (correlating to the level of cell lysis) were measured using a Packard Fusion™ α-FP HT reader (PerkinElmer, Boston, Mass.). The experiment was carried out in triplicates. FIG. 122b shows the ADCC data comparing WT and Fc variant trastuzumab against the four different Her2/neu+ cell lines. The S239D/I332E and S239D/I332E/A330L variants provide substantial ADCC enhancements over WT trastuzumab at high, moderate, and low expression levels of target antigen. This result suggests that the Fc variants of the present invention may broaden the therapeutic window of anti-cancer antibodies.

### Example 21. ADCC with NK Cells as Effector Cells

Natural killer (NK) cells are a subpopulation of cells present in PBMCs that are thought to play a significant role in ADCC. Select Fc variants were tested in a cell-based ADCC assay in which natural killer (NK) cells rather than PBMCs were used as effector cells. In this assay the release of endogenous lactose dehydrogenase (LDH), rather than EuTDA, was used to monitor cell lysis. FIG. 123 shows that the Fc variants show substantial ADCC enhancement when NK cells are used as effector cells. Furthermore, together with previous assays, the results indicate that the Fc variants of the present invention show substantial ADCC enhancements regardless of the type of effector cell or the detection method used.

### Example 22. ADCP of Fc Variants

Another important FcγR-mediated effector function is ADCP. Phagocytes such as macrophages, neutrophils, and dendritic cells, express both activating and inhibitory FcγRs. The impact of FcLIR-mediated phagocytosis on target cancer cells is two-fold. First, engulfment results in the immediate destruction of target cells, akin to ADCC. Second, FcγR-mediated phagocytosis and endocytosis are mechanisms of antigen uptake, funneling antigen into the appropriate pathways for processing and presentation that can ultimately lead to adaptive immunity (Amigorena S and Bonnerot C 1999. Fc receptors for IgG and antigen presentation on MHC class I and class II molecules. Semin Immunol 11:385-390.).

To investigate the effect of the Fc variants on ADCP, a dual fluorescent labeling strategy was used to demonstrate the capacity of WT and Fc variant antibodies to mediate phagocytosis of target cells. Monocytes were isolated from heterozygous V158/F158 FcγRIIIa human PBMCs using a Percoll gradient and differentiated into macrophages by culture with 0.1 ng/ml GM-CSF for one week. Quantitative ADCP was measured using a co-labeling strategy coupled with flow cytometry. Differentiated macrophages were detached with EDTA/PBS- and labeled with the lipophilic fluorophore, PKH26, according to the manufacturer's protocol (Sigma, St Louis, Mo.). Target cells (Sk-Br-3 for trastuzumab and WIL2-S for rituximab) were labeled with PKH67 (Sigma, St Louis, Mo.), seeded in a 96-well plate at 20,000 cells per well, and treated with the designated final concentrations of WT or Fc variant antibody. PKH26-labeled macrophages were then added to the opsonized, labeled target cells at 20,000 cells per well, and the cells were co-cultured for 18 hrs. Fluorescence was measured using dual label flow cytometry. Percent phagocytosis was determined as the number of cells co-labeled with PKH76 and PKH26 (macrophage+target) over the total number of target cells in the population (phagocytosed+non-phagocytosed) after 10,000 counts. FIG. 124a shows data comparing WT and Fc variant trastuzumab at various antibody concentrations. The results indicate that the S239D/I332E/A330L variant provides a significant enhancement in ADCP over WT trastuzumab. A similar experiment in the context of the anti-CD20 antibody rituximab also shows ADCP enhancement for the S239D/I332E and S239D/I332E/A330L variants against WIL2-S target cells (FIG. 124b).

### Example 23. Complement Binding and Activation by Fc Variants

Complement protein C1q binds to a site on Fc that is proximal to the FcγR binding site, and therefore it was prudent to determine whether the Fc variants have maintained their capacity to recruit and activate complement. The AlphaScreen assay was used to measure binding of select Fc variants to the complement protein C1q. The assay was carried out with biotinylated WT alemtuzumab antibody attached to streptavidin donor beads as described in Example 17, and using C1q coupled directly to acceptor beads. Binding data of V264I, 1332E, S239E, and V264I/I332E rituximab shown in FIG. 125a indicate that C1q binding is uncompromised. Cell-based CDC assays were also performed on select Fc variants to investigate whether Fc variants maintain the capacity to activate complement. Alamar Blue was used to monitor lysis of Fc variant and WT rituximab-opsonized WIL2-S lymphoma cells by human serum complement (Quidel, San Diego, Calif.). The data in FIG. 125b show that CDC is uncompromised for the Fc variants S239E, V264I, and V264I/I332E rituximab. In contrast, FIG. 125c shows that CDC of the Fc variant S239D/I332E/A330L is completely ablated, whereas the S239D/I332E variant mediates CDC that is comparable to WT rituximab. These results indicate that protein engineering can be used to distinguish between different effector functions. Such control will not only enable the generation of Fc polypeptides, including antibodies and Fc fusions, with properties tailored for a desired clinical outcome, but also provide a unique set of reagents with which to experimentally investigate effector function biology.

### Example 24. Enhanced B Cell Depletion in Macaques

Because of its capacity to deplete normal B cells, rituximab provides a feasible in vivo experiment with which to test our Fc variants. Periphal B cell depletion in cynomolgus monkeys has been previously reported as a suitable measure of anti-CD20 cytotoxicity (Reff, M. E et al., 1994. Depletion of B cells in vivo by a chimeric mouse human monoclonal antibody to CD20. Blood 83:435-445). The advantage of testing in this system is that monkey FcγRs, in contrast to those in mice, are highly homologous to and have similar biology as human receptors. Four variant and two WT doses were evaluated in the present study to approximate the dose required to deplete 50% of circulating B cells.

Cynomolgus monkeys (Macaca fascicularis) were injected intravenously once daily for 4 consecutive days with WT or S239D/I332E rituximab antibody. The experiment comprised 6 treatment groups of approximately 0.2, 2, 7, or 34 μg/kg (S239D/I332E) or approximately 2 or 34 μg/kg (WT control), with 3 monkeys per treatment group. Blood samples were acquired on two separate days prior to dosing (baseline) and at days 1, 2, 5, 15, and 28 following initiation of dosing. For each sample, cell populations were quantified using flow-cytometry and specific antibodies against the following marker antigens: CD2+/CD20+(all lymphocytes, sample purity/total B cells), CD20+ and CD40+(B-lymphocytes), CD3+(T-lymphocytes), CD3+/CD4+(T-helper lymphocytes), CD3+/CD8+(T-cytotoxic/suppressor lymphocytes), CD3−/CD16+ and CD3−/CD8+(Natural-killer cells), and CD3−/CD14+(Monocytes). Absolute numbers of each cell-type were determined by multiplying the proportion of cells expressing the indicated markers by the absolute lymphocyte count and/or absolute monocyte count (determined by the standard hematological analysis). Percent B cell depletion was calculated by comparing the B cell counts on any given day to the average of the two baseline measures for each animal. Data reported are group averages.

An enhanced level of B cell depletion is observed for the S239D/I332E variant relative to WT as measured by the population of CD20+(FIG. 126a) and CD40+(FIG. 126b) cells. A characteristic rebound in B cells is observed (Reff et al., 1994), followed by further reduction and gradual recovery, with the greatest level of depletion occurring at day 5. B cell level was still not fully recovered at 28 days, but completely recovered by day 84 (data not shown). Interpolation of the day 5 data at the approximate dose required for 50% B cell depletion (FIG. 126c) suggests a dose of nearly 10 μg/kg/day for WT, in good agreement with historical data (Reff et al., 1994). For the S239D/I332E variant, a dose of 0.2 μg/kg/day (0.25) is sufficient for 50% depletion, an apparent increase in potency of approximately 40-50-fold. Concerns about the potential for antibody/FcγRIIIa interactions to promote apoptosis of activated NK cells (Sulica et al., 2001. Ig-binding receptors on human NK cells as effector and regulatory surface molecules. Int Rev Immunol 20:371-414; Warren & Kinnear, 1999. Quantitative analysis of the effect of CD16 ligation on human NK cell proliferation. J Immunol 162:735-742. motivated us to also investigate the effect of the variant rituximab on NK cell levels. A dose dependent decrease in NK cells is observed in all groups as measured by the population of CD3−/CD8+(FIG. 126d) and CD3−/CD16+(FIG. 126e) cells, correlated with the degree of B cell depletion effected. No difference in NK cell reduction compared to B cell reduction is observed between WT and variant anti-CD20 antibodies. NK cell populations recovered to predose range within two weeks of the initial dose. No significant changes were observed in monocytes, T-helper lymphocytes, T-cytotoxic/suppressor lymphocytes, or total T-lymphocytes as measured by the populations of CD3−/CD14+, CD3+/CD4+, CD3+/CD8+, and CD3+ cells respectively (data not shown).

The B cell depletion experiments of the present studies have deliberately focused on the macaque system for the purpose of maintaining a high degree of homology to the human immune biology. An approximation of the dose required to deplete 50% of circulating B cells was used to evaluate the potency of the S239D/I332E variant relative to WT rituximab. The variant clearly shows increased potency relative to WT human IgG1 rituximab, consistent with its enhanced receptor affinity and ADCC in vitro, and with the observation that B cell depletion by rituximab in vivo is dominated by FcγR-mediated mechanisms (Uchida et al, 2004. The innate mononuclear phagocyte network depletes B lymphocytes through Fc receptor-dependent mechanisms during anti-CD20 antibody immunotherapy. J Exp Med 199:1659-1669; Vugmeyster & Howell, 2004. Rituximab-mediated depletion of cynomolgus monkey B cells in vitro in different matrices: possible inhibitory effect of IgG. Int Immunopharmacol 4:1117-1124.) The fold potency improvement in vivo (>40×) is less than the observed improvement for the same variant in vitro (>100×) but still quite convincing. A number of factors may contribute to the difference observed in vitro and in vivo, including for example the high concentration of non-specific IgG in the serum (Vugmeyster & Howell, 2004). Nonetheless, the in vivo experiment was undertaken because, short of a clinical trial, it is the best predictor of clinical effect. Accordingly, the capacity of the engineered Fc region to substantially enhance efficacy in the current model is significant motivation for using it or like variants in clinical trials.

### Example 25. Capacity for Testing Fc Variants in Mice

Optimization of Fc to nonhuman FcγRs may be useful for experimentally testing Fc variants in animal models. For example, when tested in mice (for example nude mice, SCID mice, xenograft mice, and/or transgenic mice), antibodies and Fc fusions that comprise Fc variants that are optimized for one or more mouse FcγRs may provide valuable information with regard to clinical efficacy, mechanism of action, and the like. In order to evaluate whether the Fc variants of the present invention may be useful in such experiments, affinity of select Fc variants for mouse FcγRIII was measured using the AlphaScreen assay. The AlphaScreen assay was carried out using biotinylated WT alemtuzumab attached to streptavidin donor beads as described in Example 17, and GST-tagged mouse FcγRIII bound to glutathione chelate acceptor beads, expressed and purified as described in Example 17. These binding data are shown in FIG. 127a for Fc variants in the context of alemtuzumab, and in FIGS. 127b and 127c in the context of trastuzumab. Results show that some Fc variants that enhance binding to human FcγRIIIa also enhance binding to mouse FcγRIII. The enhancement of mouse effector function by the Fc variants was investigated by performing the aforementioned cell-based ADCC assays using mouse rather than human PBMC's. FIG. 128 shows that the S239D/I332E/A330L trastuzumab variant provides substantial ADCC enhancement over WT in the presence of mouse immune cells. This result indicates that the Fc variants of the present invention, or other Fc variants that are optimized for nonhuman FcγRs, may find use in experiments that use animal models.

### Example 26. Validation of Fc Variants Expressed in CHO Cells

Whereas the Fc variants of the present invention were expressed in 293T cells for screening purposes, large scale production of antibodies is typically carried out by expression in Chinese Hamster Ovary (CHO) cell lines. In order to evaluate the properties of CHO-expressed Fc variants, select Fc variants and WT alemtuzumab were expressed in CHO cells and purified as described in Example 16. FIG. 129 shows AlphaScreen data comparing binding of CHO- and 293T-expressed Fc variant and WT alemtuzumab to human V158 FcγRIIIa. The results indicate that the Fc variants of the present invention show comparable FcγR binding enhancements whether expressed in 293T or CHO.

### Example 27. Enhancement of Fc Variants in Fucose Minus Strain

Combinations of the Fc variants of the present invention with other Fc modifications are contemplated with the goal of generating novel Fc polypeptides with optimized properties. It may be beneficial to combine the Fc variants of the present invention with other Fc modifications, including modifications that alter effector function or interaction with one or more Fc ligands. Such combination may provide additive, synergistic, or novel properties in Fc polypeptides. For example, a number of methods exist for engineering different glycoforms of Fc that alter effector function. Engineered glycoforms may be generated by a variety of methods known in the art, many of these techniques are based on controlling the level of fucosylated and/or bisecting oligosaccharides that are covalently attached to the Fc region. One method for engineering Fc glycoforms is to express the Fc polypeptide in a cell line that generates altered glycoforms, for example Lec-13 CHO cells. In order to investigate the properties of Fc variants combined with engineered glycoforms, WT and V209 (S239D/I332E/A330L) trastuzumab were expressed in Lec-13 CHO cells and purified as described above. FIG. 130a shows AlphaScreen binding data comparing the binding to human V158 FcγRIIIa by WT and V209 trastuzumab expressed in 293T, CHO, and Lec-13 cells. The results show that there is substantial synergy between the engineered glycoforms produced by this cell line and the Fc variants of the present invention. The cell-based ADCC assay, shown in FIG. 130b, supports this result. Together these data indicate that other Fc modifications, particularly engineered glycoforms, may be combined with the Fc variants of the present invention to generate Fc polypeptides, for example, antibodies and Fc fusions, with optimized effector functions.

### Example 28. Aglycosylated Fc Variants

As discussed, one goal of the current experiments was to obtain optimized aglycosylated Fc variants. Several Fc variants provide significant progress towards this goal. Because it is the site of glycosylation, substitution at N297 results in an aglycosylated Fc. Whereas all other Fc variants that comprise a substitution at N297 completely ablate FcγR binding, N297D/I332E has significant binding affinity for FcγRIIIa, shown in FIG. 24 and illustrated in FIG. 131. The exact reason for this result is uncertain in the absence of a high-resolution structure for this variant, although the computational screening predictions suggest that it is potentially due to a combination of new favorable Fc/FcγR interactions and favorable electrostatic properties. Indeed other electrostatic substitutions are envisioned for further optimization of aglycosylated Fc. FIG. 24 shows that other aglycosylated Fc variants such as N297D/A330Y/I332E and S239D/N297D/I332E provide binding enhancements that bring affinity for FcγRIIIa within as much as 0.4- and 0.8- respectively of glycosylated WT alemtuzumab. Combinations of these variants with other Fc variants that enhance FcγR binding are contemplated, with the goal of obtaining aglycosylated Fc variants that bind one or more FcγRs with affinity that is approximately the same as or even better than glycosylated parent Fc. Exemplary Fc variants for enhancing Fc ligand binding and/or effector function in an aglycosylated Fc polypeptide include but are not limited to: N297D, N297D/I332E, N297D/I332D, S239D/N297D, S239D/N297D/I332E, N297D/A330Y/I332E, and S239D/N297D/A330Y/I332E. The present invention of course contemplates combinations of these aglycosylated variants with other Fc variants described herein which also enhance Fc ligand binding and/or effector function.

An additional set of promising Fc variants provide stability and solubility enhancements in the absence of carbohydrate. Fc variants that comprise substitutions at positions 241, 243, 262, and 264, positions that do not mediate FcγR binding but do determine the interface between the carbohydrate and Fc, ablate FcγR binding, presumably because they perturb the conformation of the carbohydrate. In deglycosylated form, however, Fc variants F241E/F243R/V262E/V264R, F241E/F243Q/V262T/V264E, F241R/F243Q/V262T/V264R, and F241E/F243Y/V262T/V264R show stronger binding to FcγRIIIa than in glycosylated form, as shown by the AlphaScreen data in FIG. 132. This result indicates that these are key positions for optimization of the structure, stability, solubility, and function of aglycosylated Fc. Together these results suggests that protein engineering can be used to restore the favorable functional and solution properties of antibodies and Fc fusions in the absence of carbohydrate, and pave the way for aglycosylated antibodies and Fc fusions with favorable solution properties and full functionality that comprise substitutions at these and other Fc positions.

### Example 29. Preferred Variants

Taken together, the data provided in the present invention indicate that Fc variants that provide optimized FcγR binding properties also provide enhanced effector function. Substitutions at a number of positions, including but not limited to 236, 239, 246, 246, 249, 255, 258, 260, 264, 267, 268, 272, 274, 281, 283, 304, 324, 326, 327, 330, 332, 333, 334, and 334 provide promising candidates for improving the effector function and therefore the clinical properties of Fc polypeptides, including antibodies and Fc fusions. Because combinations of Fc variants of the present invention have typically resulted in additive or synergistic binding improvements, and accordingly additive or synergistic enhancements in effector function, it is anticipated that as yet unexplored combinations of the Fc variants provided in FIG. 24 will also provide favorable results. Alternative Fc variants of the present invention for enhancing Fc ligand binding and/or effector function are provided in Table 9.

TABLE 9

| | | | |
|---|---|---|---|
| G236S | S239D/I332E | S239D/K246H/I332E | S239D/K246H/T260H/I332E |
| G236A | S239D/G236A | S239D/V264I/I332E | S239D/K246H/H268D/I332E |
| S239D | S239D/G236S | S239D/S267E/I332E | S239D/K246H/H268E/I332E |
| S239E | S239D/V264I | S239D/H268D/I332E | S239D/H268D/S324G/I332E |
| S239N | S239D/H268D | S239D/H268E/I332E | S239D/H268E/S324G/I332E |
| S239Q | S239D/H268E | S239D/S298A/I332E | S239D/H268D/K326T/I332E |
| S239T | S239D/S298A | S239D/S324G/I332E | S239D/H268E/K326T/I332E |
| K246H | S239D/K326E | S239D/S324I/I332E | S239D/H268D/A330L/I332E |
| K246Y | S239D/A330L | S239D/K326T/I332E | S239D/H268E/A330L/I332E |
| D249Y | S239D/A330Y | S239D/K326E/I332E | S239D/H268D/A330Y/I332E |
| R255Y | S239D/A330I | S239D/K326D/I332E | S239D/H268E/A330Y/I332E |
| E258Y | I332E/V264I | S239D/A327D/I332E | S239D/S298A/S267E/I332E |
| T260H | I332E/H268D | S239D/A330L/I332E | S239D/S298A/H268D/I332E |
| V264I | I332E/H268E | S239D/A330Y/I332E | S239D/S298A/H268E/I332E |
| S267E | I332E/S298A | S239D/A330I/I332E | S239D/S298A/S324G/I332E |
| H268D | I332E/K326E | S239D/K334T/I332E | S239D/S298A/S324I/I332E |
| H268E | I332E/A330L | | S239D/S298A/K326T/I332E |
| E272Y | I332E/A330Y | | S239D/S298A/K326E/I332E |
| E272I | I332E/A330I | | S239D/S298A/A327D/I332E |
| E272H | I332E/G236A | | S239D/S298A/A330L/I332E |
| K274E | I332E/G236S | | S239D/S298A/A330Y/I332E |
| G281D | I332D/V264I | | S239D/K326T/A330Y/I332E |
| E283L | I332D/H268D | | S239D/K326E/A330Y/I332E |
| E283H | I332D/H268E | | S239D/K326T/A330L/I332E |
| S304T | I332D/S298A | | S239D/K326E/A330L/I332E |
| S324G | I332D/K326E | | |
| S324I | I332D/A330L | | |
| K326T | I332D/A330Y | | |
| A327D | I332D/A330I | | |
| A330Y | I332D/G236A | | |
| A330L | I332D/G236S | | |
| A330I | | | |
| I332D | | | |
| I332E | | | |
| I332N | | | |
| I332Q | | | |
| E333Y | | | |
| K334T | | | |
| K334F | | | |

### Example 30. Therapeutic Application of Fc Variants

A number of Fc variants described in the present invention have significant potential for improving the therapeutic efficacy of anticancer antibodies. For illustration purposes, a number of Fc variants of the present invention have been incorporated into the sequence of the antibody rituximab. The WT rituximab light chain and heavy chain, described in U.S. Pat. No. 5,736,137, are provided in FIGS. 133a and 133b. The improved anti-CD20 antibody sequences are provided in FIG. 133c. The improved anti-CD20 antibody sequences comprise at least non-WT amino acid selected from the group consisting of X₁, X₂, X₃, X₄, X₅, X₆, X₇, X₈, and X₉. These improved anti-CD20 antibody sequences may also comprise a substitution Z₁ and/or Z₂. The use of rituximab here is solely an example, and is not meant to constrain application of the Fc variants to this antibody or any other particular Fc polypeptide.

Table 10 depicts the positions of human Fc, the wild type residue, and the variants that are included in particular embodiments of the invention. Table 10 is based on IgG1, although as will be appreciated by those in the art, the same thing can be done to any Ig, particularly IgG2, IgG3 and IgG4.

TABLE 10

| Position | Wild Type (Human) | Variants including wild type |
|---|---|---|
| 118-220 | FX | see FIG. 1a |
| Vb(221) | D | D, K, Y |
| Vb(222) | K | K, E, Y |
| Vb(223) | T | T, E, K |
| Vb(224) | H | H, E, Y |
| Vb(225) | T | T, E, K, W |
| Fx(226) | WT | C |
| Vb(227) | P | P, E, G, K, Y |
| Vb(228) | P | P, E, G, K, Y |
| Fx(229) | (OPEN)(WT) | C |
| Vb(230) | P | P, A, E, G, Y |
| Vb(231) | A | A, E, G, K, P, Y |
| Vb(232) | P | P, E, G, K, Y |
| Vb(233) | E | A, D, F, G, H, I, K, L, M, |
| | | N, Q, R, S, T, V, W, Y |
| Vb(234) | L | L, A, D, E, F, G, H, I, K, M, |
| | | N, P, Q, R, S, T, V, W, Y |
| Vb(235) | L | L, A, D, E, F, G, H, I, K, M, |
| | | N, P, Q, R, S, T, V, W, Y |
| Vb(236) | G | G, A, D, E, F, H, I, K, L, M, |
| | | N, P, Q, R, S, T, V, W, Y |
| Vb(237) | G | G, D, E, F, H, I, K, L, M, N, |
| | | P, Q, R, S, T, V, W, Y |
| Vb(238) | P | P, D, E, F, G, H, I, K, L, M, |
| | | N, Q, R, S, T, V, W, Y |
| Vb(239) | S | S, D, E, F, G, H, I, K, L, M, |
| | | N, P, Q, R, T, V, W, Y |
| Vb(240) | V | V, A, I, M, T |
| Vb(241) | F | F, D, E, L, R, S, W, Y |
| Fx(242) | WT | L |
| Vb(243) | F | F, E, H, L, Q, R, W, Y |
| Vb(244) | P | P, H |
| Vb(245) | P | P, A |
| Vb(246) | K | K, D, E, H, Y |
| Vb(247) | P | P, G, V |
| Vb(248) | WT | K |
| Vb(249) | D | D, H, Q, Y |
| Fx(250-254) | WT | -(T-L-M-I-S)- |
| Vb(255) | R | R, E, Y |
| Fx(256-257) | WT | -(T-P)- |
| Vb(258) | E | E, H, S, Y |
| Fx(259) | WT | V |
| Vb(260) | T | T, D, E, H, Y |
| Fx(261) | WT | C |
| Vb(262) | V | V, A, E, F, I, T |
| Vb(263) | V | V, A, I, M, T |
| Vb(264) | V | V, A, D, E, F, G, H, I, K, L, |
| | | M, N, P, Q, R, S, T, W, Y |
| Vb(265) | D | D, F, G, H, I, K, L, M, N, P, |
| | | Q, R, S, T, V, W, Y |
| Vb(266) | V | V, A, I, M, T |
| Vb(267) | S | S, D, E, F, H, I, K, L, M, N, |
| | | P, Q, R, T, V, W, Y |
| Vb(268) | H | H, D, E, F, G, I, K, L, M, N, |
| | | P, Q, R, T, V, W, Y |
| Vb(269) | E | E, F, G, H, I, K, L, M, N, P, |
| | | R, S, T, V, W, Y |
| Vb(270) | D | D, F, G, H, I, L, M, P, Q, R, |
| | | S, T, W, Y |
| Vb(271) | A | A, D, E, F, G, H, I, K, L, M, |
| | | N, Q, R, S, T, V, W, Y |
| Vb(272) | E | E, D, F, G, H, I, K, L, M, P, |
| | | R, S, T, V, W, Y |
| Vb(273) | V | V, I |
| Vb(274) | K | K, D, E, F, G, H, L, M, N, P, |
| | | R, T, V, W, Y |
| Vb(275) | F | F, L, W |
| Vb(276) | N | N, D, E, F, G, H, I, L, M, P, |
| | | R, S, T, V, W, Y |
| Fx(277) | WT | W |
| Vb(278) | Y | Y, D, E, G, H, I, K, L, M, N, |
| | | P, Q, R, S, T, V, W |
| Fx(279) | WT | V |
| Vb(280) | D | D, G, K, L, P, W |
| Vb(281) | G | G, D, E, K, N, P, Q, Y |
| Vb(282) | V | V, E, G, K, P, Y |
| Vb(283) | E | E, G, H, K, L, P, R, Y |
| Vb(284) | V | V, D, E, L, N, Q, T, Y |
| Vb(285) | H | H, D, E, K, Q, W, Y |
| Vb(286) | N | N, E, G, P, Y |
| Fx(287) | WT | A |
| Vb(288) | K | K, D, E, Y |
| Fx(289) | WT | T |
| Vb(290) | K | K, D, H, L, N, W |
| Vb(291) | P | P, D, E, G, H, I, Q, T |
| Vb(292) | R | R, D, E, T, Y |
| Vb(293) | E | E, F, G, H, I, L, M, N, P, R, S, |
| | | T, V, W, Y |
| Vb(294) | E | E, F, G, H, I, K, L, M, P, R, S, |
| | | T, V, W, Y |
| Vb(295) | Q | Q, D, E, F, G, H, I, M, N, P, R, |
| | | S, T, V, W, Y |
| Vb(296) | Y | Y, A, D, E, G, H, I, K, L, M, N, |
| | | Q, R, S, T, V |
| Vb(297) | N | N, D, E, F, G, H, I, K, L, M, P, |
| | | Q, R, S, T, V, W, Y |
| Vb(298) | S | S, D, E, F, H, I, K, M, N, Q, R, |
| | | T, W, Y |
| Vb(299) | T | T, A, D, E, F, G, H, I, K, L, M, |
| | | N, P, Q, R, S, V, W, Y |
| Vb(300) | Y | Y, A, D, E, G, H, K, M, N, P, Q, |
| | | R, S, T, V, W |
| Vb(301) | R | R, D, E, H, Y |
| Vb(302) | V | V, I |
| Vb(303) | V | V, D, E, Y |
| Vb(304) | S | S, D, H, L, N, T |
| Vb(305) | V | V, E, T, Y |
| Fx(306-312) | WT | -(L-T-V-L-H-Q-D)-* |
| Vb(313) | W | W, F |
| Fx(314-316) | WT | -(L-N-G)- |
| Vb(317) | K | K, E, Q |
| Vb(318) | E | E, H, L, Q, R, Y |
| Fx(319) | WT | Y |
| Vb(320) | K | K, D, F, G, H, I, L, N, P, S, T, |
| | | V, W, Y |
| Fx(321) | WT | C |
| Vb(322) | K | K, D, F, G, H, I, P, S, T, V, W, |
| | | Y |
| Vb(323) | V | V, I |
| Vb(324) | S | S, D, F, G, H, I, L, M, P, R, T, |
| | | V, W, Y |
| Vb(325) | N | N, A, D, E, F, G, H, I, K, L, M, |
| | | P, Q, R, S, T, V, W, Y |
| Vb(326) | K | K, I, L, P, T |
| Vb(327) | A | A, D, E, F, H, I, K, L, M, N, P, |
| | | R, S, T, V, W, Y |
| Vb(328) | L | L, A, D, E, F, G, H, I, K, M, N, |
| | | P, Q, R, S, T, V, W, Y |
| Vb(329) | P | P, D, E, F, G, H, I, K, L, M, N, |
| | | Q, R, S, T, V, W, Y |
| Vb(330) | A | A, E, F, G, H, I, L, M, N, P, R, |
| | | S, T, V, W, Y |
| Vb(331) | P | P, D, F, H, I, L, M, Q, R, T, V, |
| | | W, Y |
| Vb(332) | I | I, A, D, E, F, H, K, L, M, N, P, |
| | | Q, R, S, T, V, W, Y |
| Vb(333) | E | E, F, H, I, L, M, N, P, T, Y |
| Vb(334) | K | K, F, I, L, P, T |
| Vb(335) | T | T, D, F, G, H, I, L, M, N, P, R, |
| | | S, V, W, Y |
| Vb(336) | I | I, E, K, Y |
| Vb(337) | S | S, E, H, N |

### Example 31: Protein a and FcRn Binding by Fc Variants

As discussed, bacterial proteins A and G and the neonatal Fc receptor FcRn bind to the Fc region between the Cγ2 and Cγ3 domains. Protein A is frequently employed for antibody purification, and FcRn plays a key role in antibody pharmacokinetics and transport. It was therefore important to investigate the ability of the Fc variants of the present invention to bind protein A and FcRn. The AlphaScreen™ assay was used to measure binding of select Fc variants to protein A and human FcRn using biotinylated WT alemtuzumab antibody attached to streptavidin donor beads as described in Example 2, and using protein A and FcRn coupled directly to acceptor beads. The binding data are shown in FIG. 142 for protein A and FIG. 143 for FcRn. The results indicate that the Cγ2-Cγ3 hinge region is unaffected by the Fc substitutions, and importantly that the capacity of the Fc variants to bind protein A and FcRn is uncompromised.

All cited references are herein expressly incorporated by reference in their entirety.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

### REFERENCES

Tai, et al. (2012) “Potent in vitro and in vivo activity of an Fc-engineered humanized anti-HM1.24 antibody against multiple myeloma via augmented effector function,” Blood (2012) Jan. 13, sections entitled In vivo tumor xenograft models, Plasma cell depletion study in nonhuman primates, Lenalidomide further enhances XmAb5592 induced anti-myeloma activity both in vitro and in vivo, XmAb5592 depletes blood/BM plasma cells in cynomolgus monkeys, Supp FIG. 5, FIG. 5B, Supp FIG. 6, FIGS. 6A-B.
Chu, et al. (2012) “Reduction of Total IgE by Targeted Coengagement of IgE B cell Receptor and FcgRIIb with Fc-Engineered Antibody,” J Allergy Clin Immunol, doi:10.1016/j.jaci.2011.11.029
Cemerski, et al. (2012) “Suppression of mast cell degranulation through a dual-targeting tandem IgE-IgG Fc domain biologic engineered to bind with high affinity to FcgRIIb,” Immunol Lett.
Moore, et al. (2011) “A novel bispecific antibody format enables simultaneous bivalent and monovalent co-engagement of distinct target antigens,” MAbs. November 1; 3(6). [Epub ahead of print].
Desjarlais and Lazar (2011) “Modulation of antibody effector function,” Experimental Cell Research 317(9)1278-1285.
Horton, et al. (2011) “Antibody-Mediated Coengagement of FcgRIIb and B Cell Receptor Complex Suppresses Humoral Immunity in Systemic Lupus Erythematosus,” J Immunol. 2011 Apr. 1.
Maillet, et al. (2011) “Allergic Lung Inflammation is Mediated by Soluble TNF and Attenuated by Dominant-negative TNF Biologics,” Am J Respir Cell Mol Biol. 2011 Feb. 4. [Epub ahead of print].
Horton, et al. (2010) “Fc-engineered anti-CD40 antibody enhances multiple effector functions and exhibits potent in vitro and in vivo antitumor activity against hematological malignancie Blood, July 2010; doi:10,”1182/blood-2010-01-265280, sections entitled In vivo tumor xenograft models, Statistical analysis, XmAbCD40 improves survival of mice bearing disseminated Ramos lymphoma, XmAbCD40 inhibits growth of established subcutaneous Ramos tumors, FIGS. 5, 6A, 6B, 6C.
Moore, et al. (2010) “Engineered Fc variant antibodies with enhanced ability to recruit complement and mediate effector functions,” MAbs 2(2):181-189.
Awan, et al. (2010) “CD19 targeting of chronic lymphocytic leukemia with a novel Fc-domain—engineered monoclonal antibody,” Blood 115(6): 1204-1213.
Zalevsky, et al. (2010) “Enhanced antibody half-life improves in vivo activity,” Nat Biotechnol 28(2):157-9.
Bernett, et al. (2010) “Engineering Fully Human Monoclonal Antibodies from Murine Variable Regions,” J Mol Biol 396(5):1474-90.
Zalevsky, et al. (2009) “The impact of Fc engineering on an anti-CD19 antibody: increased Fc gamma receptor affinity enhances B-cell clearing in nonhuman primates,” Blood 113:3735-3743.
Horton, et al. (2008) “Potent In vitro and In vivo Activity of an Fc-Engineered Anti-CD19,” Cancer Research 68(19):8049-8057, sections entitled Lymphoma xenograft models, Data analysis, XmAb5574 inhibits lymphoma growth in SCID mouse xenograft models, In vivo B Cell Depletion, FIGS. 6C, S3.
Chu, et al. (2008) “Inhibition of B cell receptor-mediated activation of primary human B cells by coengagement of CD19 and FcgammaRIIb with Fc-engineered antibodies,” Molecular Immunology 45(15):3926-33.
Richards, et al. (2008) “Optimization of antibody binding to FcgRIIa enhances macrophage phagocytosis of tumor cells,” Molecular Cancer Therapeutics 7: 2517-2527.
Desjarlais, et al. (2007) “Optimizing engagement of the immune system by anti-tumor antibodies: an engineer's perspective,” Drug Discovery Today 12(21-22): 898-910.
Zhu, et al. (2007) “Industrial Production of Therapeutic Proteins: Cell Lines, Cell Culture, and Purification,” Industrial Chemistry and Biotechnology, Kent and Riegel, eds., 11th Edition, Chapter 32, p 1421-1448, Springer Press, The Netherlands.
Zalevsky, et al. (2007) “Dominant-Negative Inhibitors of Soluble TNF Attenuate Experimental Arthritis without Suppressing Innate Immunity to Infection,” The Journal of Immunology 179, 1872-1883.
Lazar, et al. (2007) “A molecular immunology approach to antibody humanization and functional optimization,” Molecular Immunology 44, 1986-1998.
Lazar, et al. (2006) “Engineered Antibody Fc Variants with Enhanced Effector Function,” Proc Natl Acad Sci USA 103(11) 4005-4010, sections entitled Designed Fc Variants Mediate Enhanced B Cell Depletion in Macaques, FIGS. 7A-D.
Barbos, et al. (2006) “Clinical link between MHC class II haplotype and interferon-beta (IFN-β) immunogenicity,” Clinical Immunology, 118, 42-50.
Szymkowski (2005) “Creating the next generation of protein therapeutics through rational drug design,” Current Opinion in Drug Discovery & Development 8(5) 590-600.
Vielmetter, et al. (2005) “Data management solutions for protein therapeutic research and development,” Drug Discovery Today: Biosilico, 10(15) 1065-1071.
Szymkowski (2005) “Timely lessons for target-based discovery of anti-inflammatory drugs,” Drug Discovery Today, 109(1) 14-17.
Szymkowski (2004) “Rational optimization of proteins as drugs: A new era of ‘medicinal biology’,” Drug Discovery Today, 9(9) 381-383.
Zhukovsky and Morse (2004) “Pleiotropy: the BAFFling truth of the TNF superfamily,” Drug Discovery Today: Targets, vol. 3, No. 6, pp. 225-227.
Chirino and Mire-Sluis (2004) “Characterizing biological products and assessing comparability following manufacturing changes,” Nature Biotechnology, 22(11) 1383-91.
Zhukovsky, et al. (2004) “TNF ligands: is TALL-1 a trimer or a virus-like cluster?” Nature, 427(6973) 413-4.
Chirino, et al. (2004) “Minimizing the immunogenicity of protein therapeutics,” Drug Discovery Today, 9(2) 82-90.
Steed, et al. (2003) “Inactivation of TNF Signaling by Rationally Designed Dominant-Negative TNF Variants,” Science, 301, 1895-1898(Full Text).
Lazar, et al. (2003) “Designing proteins for therapeutic applications,” Curr Opin Struct Biol, 13(4) 513-8.
Szymkowski (2003) “Chemical genomics versus orthodox drug development,” Drug Discovery Today, 8(4) 157-159.
Marshall, et al. (2003) “Rational design and engineering of therapeutic proteins,” Drug Discovery Today, 8(5) 212-2.
Filikov, et al. (2002) “Computational stabilization of human growth hormone,” Protein Science, 11, 1452.
Hayes, et al. (2002) “Combining computational and experimental screening for rapid optimization of protein properties,” Proc Natl Acad Sci USA, 99, 15926.
Luo, et al. (2002) “Development of a cytokine analog with enhanced stability using computational ultrahigh throughput screening,” Protein Science, 11(5) 1218.
Dahiyat (1999) “In silico design for protein stabilization,” Curr. Op. Biotechnol., 10, 387.
Malakauskas and Mayo (1998) “Design, structure and stability of a hyperthermophilic protein variant,” Nature Struc. Biol., 5, 470.
Dahiyat, et al. (1997) “Automated design of the surface of protein helices,” Protein Sci., 6, 1333.
Dahiyat and Mayo (1997) “Probing the role of packing specificity in protein design,” Proc Natl Acad Sci USA, 94, 10172.
Dahiyat and Mayo (1997) “De novo protein design: fully automated sequence selection,” Science, 278, 82.
Dahiyat and Mayo (1996) “Protein design automation,” Protein Sci., 5, 895.

1-15. (canceled)

16. An antibody having specificity for HIV gp120, said antibody comprising an Fc variant of a human IgG1 Fc polypeptide, wherein said Fc variant comprises amino acid modifications S239D, A330L, and I332E in the Fc region of said human IgG1 Fc polypeptide, wherein numbering is according to the EU index.

17. A nucleic acid encoding an antibody according to claim 16.

18. An expression vector comprising the nucleic acid according to claim 17.

19. A host cell comprising the expression vector according to claim 18.

20. A method of making an antibody, said method comprising culturing a host cell according to claim 19 under conditions appropriate for expression of the antibody.

21. A method of treating a patient having human immunodeficiency virus (HIV) comprising administering to said patient a composition comprising an antibody according to claim 1.
